(19) United States
(12) Patent Application Publication
(10) Pub. No.: US 2011/0201599 A1
(43) Pub. Date: Aug. 18, 2011

(54) CDK Modulators

(75) Inventors: Suleyman Bahceci, San Mateo, CA (US); Bryan Chan, San Carlos, CA (US); Diva Sze-Ming Chan, Emeryville, CA (US); Jeff Chen, San Francisco, CA (US); Timothy Patrick Forsyth, Hayward, CA (US); Maurizio Franzini, Redwood City, CA (US); Vasu Jammalamadaka, Pleasonton, CA (US); Joon Won Jeong, Belmont, CA (US); Lisa Renee Jones, Belmont, CA (US); Ryan Michael Kelley, Pacifica, CA (US); Moon Hwan Kim, Palo Alto, CA (US); James W. Leahy, San Leandro, CA (US); Morrison B. Mac, San Francisco, CA (US); Robin Tammie Noguchi, San Bruno, CA (US); Pallavi Rao, Mountain View, CA (US); Brian Hugh Ridgway, Belmont, CA (US); Wei Xu, Danville, CA (US); Yong Wang, Foster City, CA (US)

(73) Assignee: EXELIXIS, INC., South San Francisco, CA (US)

(21) Appl. No.: 13/002,195

(22) PCT Filed: Jul. 2, 2009
(86) PCT No.: PCT/US09/49637

§ 371 Date: Apr. 13, 2011

Related U.S. Application Data
(60) Provisional application No. 61/134,047, filed on Jul. 3, 2008.

(51) Int. Cl. A61K 31/55; C07D 471/04; C07D 401/14; C07D 403/14; C07D 413/00; C07D 413/14; C12N 5/071; A61K 31/437; A61K 31/505; A61K 31/497; A61K 31/5377; A61P 35/00; A61P 35/02; A61P 37/00

(52) U.S. Cl. 514/217.06; 546/113; 544/331; 544/295; 544/122; 540/598; 435/375; 514/300; 514/275; 514/252.14; 514/235.8

(57) ABSTRACT

A compound according to Formula I: or a pharmaceutically-acceptable salt thereof, wherein R1, R3, A, B and D are as defined in the specification; pharmaceutical compositions thereof, and methods of use thereof.

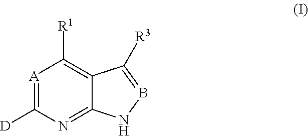

### FIELD OF THE INVENTION

This disclosure relates to certain compounds. In particular, this disclosure relates to compounds useful as modulators, and more specifically, modulators of CDK.

### BACKGROUND OF THE INVENTION

CDK (cyclin-dependent kinase) includes CDK1, CDK2, CDK4, CDK7, CDK8, and/or CDK9. CDK9 (cyclin-dependent kinase 9) is a cdc2-related serine-threonine kinase protein which appears to be involved in regulating several physiological processes with its partners of the cyclin T family (T1, T2a and T2b). In addition, CDK8/cyclin C, CDK1/cyclin B, and CDK2/cyclin E have been shown to phosphorylate the carboxyl-terminal domain in vitro. Unlike the majority of the cdc2-like kinases, CDK9 activity is not cell cycle-regulated. CDK9 acts preferentially in processes different from cell-cycle regulation, such as differentiation of several cell types (ex. monocytes and lymphocytes) thus implicating it may exert a functioning control over various differentiative pathways. The catalytic protein complexation with its regulatory subunit cyclin T was shown to be responsible for the kinase activity associated with the Positive Transcription Elongation Factor B (P-TEFB) complex. It is specifically responsible for the phosphorylation of the serine 2 residues in the C-terminal domain (CTD) of the largest subunit of RNA-Polymerase II (RNAP II) implicating activity also in the transcription process via promotion of elongation and is upregulated upon exposure to various stresses. CDK9 (cyclin-dependent kinase-9, also known as C-2K, PITALRE and CDC2L4) was cloned and later mapped to chromosome 9q34.1, a region associated with abnormalities and allelic losses in several malignancies (Best et al., Biochem. Biophys. Res. Commun., 1995, 208, 562-568; Bullrich et al., Cancer Res., 1995, 55, 1199-1205; Grana et al., Proc. Natl. Acad. Sci. U.S.A., 1994, 91, 3834-3838). Nucleic acid sequences encoding CDK9 are disclosed and claimed in U.S. Pat. No. 6,162,612 and the corresponding PCT publication WO 96/28555 (Giordano, 2000; Giordano, 1996).

CDK9 is the catalytic subunit of positive elongation factor B (P-TEFb), a transcription factor that stimulates RNA polymerase II elongation (Mancebo et al., Genes Dev., 1997, 11, 2633-2644; Zhu et al., Genes Dev., 1997, 11, 2622-2632). CDK9 also acts as the catalytic subunit for TAK (Tat-associated kinase) which binds to the human immunodeficiency virus (HIV) types 1 and 2 (Yang et al., Proc. Natl. Acad. Sci. U.S.A., 1997, 94, 12331-12336). Because Tat and TAK are essential for efficient HIV replication, regulation of CDK9 expression is likely to be an important issue with regard to viral pathogenesis (Liu and Rice, Gene, 2000, 252, 51-59). Additionally, CDK9 function is induced in activated T lymphocytes and promonocytic cell lines, suggesting that CDK9 may play a role in normal lymphocyte and monocyte/macrophage physiology. Therefore, regulation of CDK9 expression may also play a role with regard to immune cell function (Garriga et al., Oncogene, 1998, 17, 3093-3102; Liu and Rice, Gene, 2000, 252, 51-59).

There remains a need for inhibitors of CDK, such as CDK9, for the treatment of CDK-mediated conditions, such as CDK9 mediated diseases.

### SUMMARY OF THE INVENTION

One aspect of this disclosure relates to one or more compounds according to Formula I, IA, IB, IC, II, III, IV, V, VI or VII, as described in the specification, or pharmaceutically acceptable salts thereof.

Another aspect of this disclosure relates to a pharmaceutical composition, comprising the compound according to Formula I, IA, IB, IC, II, III, IV, V, VI or VII, as described in the specification, or pharmaceutically acceptable salt thereof, and a pharmaceutically acceptable carrier, excipient or diluent.

Another aspect of this disclosure relates to a method of modulating CDK in a cell, comprising contacting the cell, in which inhibition of CDK is desired, with a compound according to Formula I, IA, IB, IC, II, III, IV, V, VI or VII, as described in the specification, or a pharmaceutically acceptable salt thereof.

Another aspect of this disclosure relates to a method of modulating CDK in a cell, comprising contacting a cell in which inhibition of CDK is desired with a pharmaceutical composition, comprising the compound according to Formula I, IA, IB, IC, II, III, IV, V, VI or VII, as described in the specification, or a pharmaceutically acceptable salt thereof, and a pharmaceutically acceptable carrier, excipient or diluent.

Another aspect of this disclosure relates to a method of inhibiting CDK in a cell, comprising contacting the cell, in which inhibition of CDK is desired, with a compound according to Formula I, IA, IB, IC, II, III, IV, V, VI or VII, as described in the specification, or a pharmaceutically acceptable salt thereof.

Another aspect of this disclosure relates to a method of inhibiting CDK in a cell, comprising contacting a cell in which inhibition of CDK is desired with a pharmaceutical composition, comprising the compound according to Formula I, IA, IB, IC, II, III, IV, V, VI or VII, as described in the specification, or a pharmaceutically acceptable salt thereof, and a pharmaceutically acceptable carrier, excipient or diluent.

Another aspect of this disclosure relates to a method of treating one of the diseases or conditions disclosed herein that involves CDK, comprising administering to an animal, in need of the treatment, the compound according to Formula I, IA, IB, IC, II, III, IV, V, VI or VII, as described in the specification, or a pharmaceutically acceptable salt thereof, optionally in combination with the one or more additional therapeutic agents or therapies disclosed hererin.

There are many different aspects of the compounds, pharmaceutical compositions thereof, and methods of use thereof, as described hereinbelow, and each aspect is non-limiting in regard to the scope of the invention. The transitional term “comprising” as used herein, which is synonymous with “including,” “containing,” or “characterized by,” is inclusive or open-ended and does not exclude additional, unrecited elements or method steps.

### DETAILED DESCRIPTION OF THE INVENTION

Disclosed herein are compounds according to Formula I,

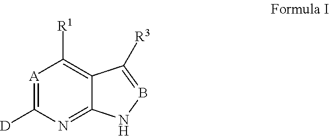

or a pharmaceutically-acceptable salt thereof, wherein:

R¹ is selected from H, halo, —NR⁴R⁵, —(C₁-C₄)alky-NR⁸R⁹, —O—(C₁-C₄)alky-NR⁸R⁹, heteroaryl optionally substituted with amino, hydroxyl, alkoxyl or aryl-(C₁-C₃)alkyl, (5-6 membered)heterocycloalkyl optionally substituted with hydroxyl, amino, alkynyl substituted with hydroxyl or —(CH₂)₀₋₃—NR⁸R⁹, phenyl substituted with —CF₃ or cyano, alkyl substituted with hydroxyl, and R^{xa};

R², when A is CR², is selected from H, halo, alkyl, —NR⁸R⁹, alkoxy, alkoxyalkoxy, dialkylaminoalkoxy, and phenyl; or R¹ and R², together with the atoms to which they are attached, join together to form a heterocycloalkyl or heteroaryl group, wherein the heterocycloalkyl or heteroaryl groups are each optionally substituted with arylalkyl or arylalkoxyalkoxy;

R³ is a (5-9 membered)heteroaryl or phenyl, wherein the (5-9 membered)heteroaryl or phenyl are each optionally substituted with 1 or 2 substituents independently selected from amine, alkoxy, hydroxyl, amine, alkyl, aminocarbonyl and halo;

A is selected from N and CR²;

B is selected from N, CH and CR^{xb};

D is selected from H or OH;

R⁴ is selected from H, alkyl optionally substituted with 1, 2 or 3 groups independently selected from hydroxyl, amino, halo, alkoxy, alkylamino, dialkylamino and dialkylaminoalkyl, alkoxycarbonyl, alkoxyalkyl, oxo, —CN, dialkylaminocarbonylalkyl optionally substituted with hydroxyl, cycloalkyl optionally substituted with amine, aryl optionally substituted with amine, arylalkyl, heteroarylalkyl optionally substituted with alkyl, heterocycloalkylalkyl and heterocycloalkyl optionally substituted with 1, 2 or 3 substituents independently selected from alkyl, hydroxyl, amino, and hydroxyalkyl;

R⁵ is selected from alkyl optionally substituted with 1, 2 or 3 groups independently selected from hydroxyl, amino, halo, alkoxy, alkylamino, dialkylamino and dialkylaminoalkyl, alkoxycarbonyl, alkoxyalkyl, oxo, —CN, dialkylaminocarbonylalkyl optionally substituted with hydroxyl, cycloalkyl optionally substituted with amine, aryl optionally substituted with amine, arylalkyl, heteroarylalkyl optionally substituted with alkyl, heterocycloalkylalkyl and heterocycloalkyl optionally substituted with 1, 2 or 3 substituents independently selected from alkyl, hydroxyl, amino, and hydroxyalkyl;

or R⁴ and R⁵, together with the nitrogen to which they are both attached, join together to form a heterocycloalkyl or heteroaryl group, wherein the heteroaryl group is optionally substituted with 1-3 substituents selected from alkyl, halo alkoxy, amino, alkylamino, dialkylamino, aminoalkyl, aminoalkylamino, dialkylaminoalkyl, hydroxyl, hydroxyalkyl, —CF₃, alkylcarbonylamino, —C(O)—N(R⁸)R⁹ and —S(O₂)—NH₂, and the heterocycloalkyl group is optionally substituted with 1, 2 or 3 substituents independently selected from halo, alkyl optionally substituted with 1-3 halo, carboxyalkyl, amino, hydroxyl, hydroxyalkyl, alkoxy optionally substituted with 1-3 halo, alkoxyalkyl optionally substituted with 1-3 halo, oxo, —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with —NR⁸R⁹, —(CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, alkoxycarbonyl and aminoalkyl;

provided that the optionally substituted heterocycloalkyl group cannot be substituted with more than one group selected from —(CH₂)₀₋₃-heterocycloalkyl optionally substituted with alkyl, ═N(H)—OH, —S(O₂)—NH₂, oxo, —N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with alkyl, (CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, and alkoxycarbonyl,

and provided that the optionally substituted heteroaryl group cannot be substituted with more than one alkylamino, dialkylamino, aminoalkyl, aminoalkylamino, dialkylaminoalkyl, alkylcarbonylamino, —C(O)—N(R⁸)R⁹ and —S(O₂)—NH₂;

R⁶ is selected from H and alkyl;

R⁷ is selected from H, hydroxyalkyl, alkylaminoalkylcarbonyl, dialkylaminoalkylcarbonyl, and alkyl optionally substituted with 1-3 halo;

R⁸ is selected from H and alkyl;

R⁹ is selected from H and alkyl;

R¹⁰ is selected from H, halo and alkyl;

R^{xa} is selected from halo, aryl or heteroaryl, wherein said aryl or heteroaryl is optionally substituted with a group selected from amino, aminocarbonyl, alkylaminocarbonyl, alkylcarbonylamino, dialkylaminocarbonyl, alkyl, haloalkyl, —S(O₂)—NH₂, and hydroxyalkyl;

R^{xb} is an aryl or heteroaryl, wherein said aryl or heteroaryl are each optionally substituted with a group selected from amino, halo, alkyl, hydroxyalkyl and —S(O₂)—N(H)—(CH₂)₀₋₃—R^{xc}; and

R^{xc} is selected from H, OH, aryl and NH₂,

provided that R¹ can be H or halo only when either (1) R³ is pyrimidin-2-amine or thiazole optionally substituted with an amine, or (2) when A is CR² and R² is —NR⁴R⁵, amino, alkoxy, alkoxyalkoxy, dialkylaminoalkoxy or phenyl.

Another embodiment of the compound according to Formula I relates to a compound of Formula IA or Formula IB:

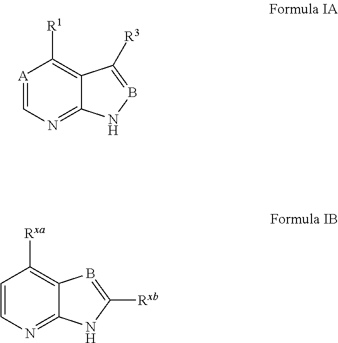

or a pharmaceutically-acceptable salt thereof, wherein:

R¹ is selected from H, halo, —NR⁴R⁵, —(C₁-C₄)alky-NR⁸R⁹, —O—(C₁-C₄)alky-NR⁸R⁹, heteroaryl optionally substituted with amino, hydroxyl, alkoxyl or aryl-(C₁-C₃)alkyl, (5-6 membered)heterocycloalkyl optionally substituted with hydroxyl, amino, alkynyl substituted with hydroxyl or —(CH₂)₀₋₃—NR⁸R⁹, phenyl substituted with —CF₃ or cyano, and alkyl substituted with hydroxyl;

R², when A is CR², is selected from H, halo, alkyl, —NR⁸R⁹, alkoxy, alkoxyalkoxy, dialkylaminoalkoxy, and phenyl;

or R¹ and R², together with the atoms to which they are attached, join together to form a heterocycloalkyl or heteroaryl group, wherein the heterocycloalkyl or heteroaryl groups are each optionally substituted with arylalkyl or arylalkoxyalkoxy;

R³ is a (5-9 membered)heteroaryl or phenyl, wherein the (5-9 membered)heteroaryl or phenyl are each optionally substituted with 1 or 2 substituents independently selected from amine, alkoxy, hydroxyl, alkyl, aminocarbonyl and halo;

A is selected from N and CR²;

B is independently selected from N and CH;

R⁴ is selected from H, alkyl optionally substituted with 1, 2 or 3 groups independently selected from hydroxyl, amino, halo, alkoxy, alkylamino, dialkylamino and dialkylaminoalkyl, alkoxycarbonyl, alkoxyalkyl, oxo, —CN, dialkylaminocarbonylalkyl optionally substituted with hydroxyl, cycloalkyl optionally substituted with amine, aryl optionally substituted with amine, arylalkyl, heteroarylalkyl optionally substituted with alkyl, heterocycloalkylalkyl and heterocycloalkyl optionally substituted with 1, 2 or 3 substituents independently selected from alkyl, hydroxyl, amino, and hydroxyalkyl;

R⁵ is selected from alkyl optionally substituted with 1, 2 or 3 groups independently selected from hydroxyl, amino, halo, alkoxy, alkylamino, dialkylamino and dialkylaminoalkyl, alkoxycarbonyl, alkoxyalkyl, oxo, —CN, dialkylaminocarbonylalkyl optionally substituted with hydroxyl, cycloalkyl optionally substituted with amine, aryl optionally substituted with amine, arylalkyl, heteroarylalkyl optionally substituted with alkyl, heterocycloalkylalkyl and heterocycloalkyl optionally substituted with 1, 2 or 3 substituents independently selected from alkyl, hydroxyl, amino, and hydroxyalkyl;

or R⁴ and R⁵, together with the nitrogen to which they are both attached, join together to form a heterocycloalkyl or heteroaryl group, wherein the heteroaryl group is optionally substituted with 1-3 substituents selected from alkyl, halo alkoxy, amino, alkylamino, dialkylamino, aminoalkyl, aminoalkylamino, dialkylaminoalkyl, hydroxyl, hydroxyalkyl, —CF₃, alkylcarbonylamino, —C(O)—N(R⁸)R⁹ and —S(O₂)—NH₂, and the heterocycloalkyl group is optionally substituted with 1, 2 or 3 substituents independently selected from halo, alkyl optionally substituted with 1-3 halo, carboxyalkyl, amino, hydroxyl, hydroxyalkyl, alkoxy optionally substituted with 1-3 halo, alkoxyalkyl optionally substituted with 1-3 halo, oxo, —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with —NR⁸R⁹, —(CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, alkoxycarbonyl and aminoalkyl;

provided that the optionally substituted heterocycloalkyl group cannot be substituted with more than one group selected from —(CH₂)₀₋₃-heterocycloalkyl optionally substituted with alkyl, ═N(H)—OH, —S(O₂)—NH₂, oxo, —N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with alkyl, (CH₂)₀₋₃—N(R⁶)R⁷, —C(O)—N(R⁸)R⁹, alkyl-2C(O)N(H)(alkyl)-, aryl, arylalkyl optionally substituted with 1 or 2 groups selected from halo and alkyl, and alkoxycarbonyl,

and provided that the optionally substituted heteroaryl group cannot be substituted with more than one alkylamino, dialkylamino, aminoalkyl, aminoalkylamino, dialkylaminoalkyl, alkylcarbonylamino, —C(O)—N(R⁸)R⁹ and —S(O₂)—NH₂;

R⁶ is selected from H and alkyl;

R⁷ is selected from H, hydroxyalkyl, alkylaminoalkylcarbonyl, dialkylaminoalkylcarbonyl, and alkyl optionally substituted with 1-3 halo;

R⁸ is selected from H and alkyl;

R⁹ is selected from H and alkyl;

R¹⁰ is selected from H, halo and alkyl;

R^{xa} is selected from halo, aryl or heteroaryl, wherein said aryl or heteroaryl is optionally substituted with a group selected from amino, aminocarbonyl, alkylaminocarbonyl, alkylcarbonylamino, dialkylaminocarbonyl, alkyl, haloalkyl, —S(O₂)—NH₂, and hydroxyalkyl;

R^{xb} is an aryl or heteroaryl, wherein said aryl or heteroaryl are each optionally substituted with a group selected from amino, halo, alkyl, hydroxyalkyl and —S(O₂)—N(H)—(CH₂)₀₋₃—R^{xc}; and

R^{xc} is selected from H, OH, aryl and NH₂,

provided that R¹ can be H or halo only when either (1) R³ is pyrimidin-2-amine or thiazole optionally substituted with an amine, or (2) when A is CR² and R² is —NR⁴R⁵, amino, alkoxy, alkoxyalkoxy, dialkylaminoalkoxy or phenyl.

Another embodiment relates to a compound of Formula I or IA, or a pharmaceutically acceptable salt thereof, wherein R¹ is selected from —NR⁴R⁵, —(C₁-C₄)alky-NR⁸R⁹, —O—(C₁-C₄)alky-NR⁸R⁹, heteroaryl optionally substituted with amino, hydroxyl, alkoxyl or aryl-(C₁-C₃)alkyl, (5-6 membered)heterocycloalkyl optionally substituted with hydroxyl, amino, alkynyl substituted with hydroxyl or —(CH₂)₀₋₃—NR⁸R⁹, phenyl substituted with —CF₃ or cyano, and alkyl substituted with hydroxyl, wherein R⁴, R⁵, R⁸ and R⁹ are as defined in any of the embodiments above.

Another embodiment relates to a compound of Formula I or IA, or a pharmaceutically acceptable salt thereof, wherein:

R¹ is selected from —NR⁴R⁵, —(C₁-C₄)alky-NR⁸R⁹, —O—(C₁-C₄)alky-NR⁸R⁹, heteroaryl optionally substituted with amino, hydroxyl, alkoxyl or aryl-(C₁-C₃)alkyl, (5-6 membered)heterocycloalkyl optionally substituted with hydroxyl, amino, and phenyl, wherein R⁴, R⁵, R⁸ and R⁹ are as defined in any of the embodiments above; and

R³ is phenyl, pyridine or pyrimidine, wherein the phenyl, pyridine or pyrimidine is optionally substituted with 1 or 2 substituents independently selected from alkoxy, hydroxyl, amine, alkyl and aminocarbonyl.

Another embodiment relates to a compound of Formula I or IA, or a pharmaceutically acceptable salt thereof, wherein:

R¹ is selected from NR⁴R⁵, —(C₁-C₄)alky-NR⁸R⁹, —O—(C₁-C₄)alky-NR⁸R⁹, triazolyl optionally substituted with phenylmethyl, 1H-pyrrolo[2,3-b]pyridinyl, 1H-indolyl, pyridinyl optionally substituted with amino or methoxy, pyrazolyl, pyrimidinyl optionally substituted with amino, (5-6 membered)heterocycloalkyl optionally substituted with hydroxyl, and amino;

R³ is phenyl, pyridine or pyrimidine, wherein the phenyl, pyridine or pyrimidine is optionally substituted with 1 or 2 substituents independently selected from amine, alkoxy, hydroxyl, alkyl and aminocarbonyl;

R⁴ is selected from H, alkyl optionally substituted with 1, 2 or 3 groups independently selected from hydroxyl, amino, halo, alkoxy, alkylamino, dialkylamino and dialkylaminoalkyl, alkoxycarbonyl, alkoxyalkyl, oxo, —CN, dialkylaminocarbonylalkyl optionally substituted with hydroxyl, cycloalkyl optionally substituted with amine, aryl optionally substituted with amine, arylalkyl, heteroarylalkyl optionally substituted with alkyl, heterocycloalkylalkyl and heterocycloalkyl optionally substituted with 1, 2 or 3 substituents independently selected from alkyl, hydroxyl, amino, and hydroxyalkyl;

R⁵ is selected from alkyl optionally substituted with 1, 2 or 3 groups independently selected from hydroxyl, amino, halo, alkoxy, alkylamino, dialkylamino and dialkylaminoalkyl, alkoxycarbonyl, alkoxyalkyl, oxo, —CN, dialkylaminocarbonylalkyl optionally substituted with hydroxyl, cycloalkyl optionally substituted with amine, aryl optionally substituted with amine, arylalkyl, heteroarylalkyl optionally substituted with alkyl, heterocycloalkylalkyl and heterocycloalkyl optionally substituted with 1, 2 or 3 substituents independently selected from alkyl, hydroxyl, amino, and hydroxyalkyl;

or R⁴ and R⁵, together with the nitrogen to which they are both attached, join together to form a heteroaryl group or a heterocycloalkyl group, wherein the heteroaryl group is selected from (1) isoindolinyl, (2) 1,2,3,4-tetrahydroisoquinolinyl optionally substituted with hydroxyl, and (3) indolinyl optionally substituted with hydroxyalkyl, and wherein the heterocycloalkyl group is selected from morpholinyl, piperidinyl, piperazinyl, pyrrolidinyl, imidazolidinyl, 3-azabicyclo[3.1.0]hexyl, 1,2,3,6-tetrahydropyridinyl, hexahydrocyclopenta[c]pyrrol-2(1H)-yl, hexahydrocyclopenta[c]pyrrol-5(1H)-yl, hexahydropyrrolo[3,4-c]pyrrol-2(1 (4aR,8aR)-octahydro-1H-pyrido[3,4-b][1,4]oxazinyl, hexahydropyrrolo[3,4-b]pyrrol-2(1 hexahydropyrrolo[3,4-b]pyrrol-5(1 thiazolidinyl, tetrahydro-3aH[1,3]dioxolo[4,5-c]pyrrolyl, hexahydropyrrolo[1,2-a]pyrazin-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from halo, alkyl optionally substituted with 1-3 halo, carboxyalkyl, amino, hydroxyl, hydroxyalkyl, alkoxy optionally substituted with 1-3 halo, alkoxyalkyl optionally substituted with 1-3 halo, —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, (C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with NR⁸R⁹, —(CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, alkoxycarbonyl and aminoalkyl, provided that the optionally substituted heterocycloalkyl group cannot be substituted with more than one group selected from —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with alkyl, (CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, and alkoxycarbonyl; and

R⁶, R⁷, R⁸, R⁹, and R¹⁰ are as defined above.

Another embodiment relates to a compound of Formula I or IA, or a pharmaceutically acceptable salt thereof, wherein:

R¹ is selected from NR⁴R⁵, —(C₁-C₄)alky-NR⁸R⁹, —O—(C₁-C₄)alky-NR⁸R⁹, triazolyl optionally substituted with phenylmethyl, 1H-pyrrolo[2,3-b]pyridinyl, 1H-indolyl, pyridinyl optionally substituted with amino or methoxy, pyrazolyl, pyrimidinyl optionally substituted with amino, (5-6 membered)heterocycloalkyl optionally substituted with hydroxyl, and amino;

R³ is phenyl, pyridine or pyrimidine, wherein the phenyl, pyridine or pyrimidine is optionally substituted with 1 or 2 substituents independently selected from amine, alkoxy, hydroxyl, alkyl and aminocarbonyl;

R⁴ is selected from H, 1-methylethyl, methyl, ethyl, propyl, 2-methylpropyl, phenylethyl, dimethylaminoethyl, methoxymethyl, piperidinyl optionally substituted with methyl, cyclopentyl, dimethylaminocarbonylmethyl, ethylpyrrolidinylmethyl, piperidinylethyl, methylaminoethyl, aminoethyl, pyrrolidinylethyl, 2-propane-1,3 diol, phenyl, carboxymethyl, dimethylaminopropyl, dimethylaminopropan-2-yl, dimethylaminopropan-1-ol-2-yl, methylaminopropyl, aminocyclohexyl, methoxyethyl, (2R)-pyrrolidinylmethyl, (2S)-pyrrolidinylmethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino, (R)-benzyl 2-amino-3-hydroxypropylcarbamate and methoxymethyl;

R⁵ is selected from is selected from propyl, 1-methylethyl, methyl, ethyl, propyl, 2-methylpropyl, phenylethyl, dimethylaminoethyl, methoxymethyl, piperidinyl optionally substituted with methyl, cyclopentyl, dimethylaminocarbonylmethyl, ethylpyrrolidinylmethyl, piperidinylethyl, methylaminoethyl, aminoethyl, pyrrolidinylethyl, 2-propane-1,3 diol, phen8yl, carboxymethyl, dimethylaminopropyl, dimethylaminopropan-2-yl, dimethylaminopropan-1-ol-2-yl, methylaminopropyl, aminocyclohexyl, methoxyethyl, (2R)-pyrrolidinylmethyl, (2S)-pyrrolidinylmethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino, (R)-benzyl 2-amino-3-hydroxypropylcarbamate and methoxymethyl;

or R⁴ and R⁵, together with the nitrogen to which they are both attached, join together to form a heteroaryl group or a heterocycloalkyl group, wherein the heteroaryl group is selected from (1) isoindolinyl, (2) 1,2,3,4-tetrahydroisoquinolinyl optionally substituted with hydroxyl, and (3) indolinyl optionally substituted with hydroxyalkyl, and wherein the heterocycloalkyl group is selected from morpholinyl, piperidinyl, piperazinyl, pyrrolidinyl, imidazolidinyl, 3-azabicyclo[3.1.0]hexyl, 1,2,3,6-tetrahydropyridinyl, hexahydrocyclopenta[c]pyrrol-2(1H)-yl, hexahy2drocyclopenta[c]pyrrol-5(1H)-yl, hexahydropyrrolo[3,4-c]pyrrol-2(1H)-yl), (4aR,8aR)-octahydro-1H-pyrido[3,4-b][1,4]oxazinyl, hexahydropyrrolo[3,4-b]pyrrol-2(1H)-yl), hexahydropyrrolo[3,4-b]pyrrol-5(1H)-yl), thiazolidinyl, tetrahydro-3aH[1,3]dioxolo[4,5-c]pyrrolyl, hexahydropyrrolo[1,2-a]pyrazin-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from halo, alkyl optionally substituted with 1-3 halo, carboxyalkyl, amino, hydroxyl, hydroxyalkyl, alkoxy optionally substituted with 1-3 halo, alkoxyalkyl optionally substituted with 1-3 halo, —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, (C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with NR⁸R⁹, —(CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, alkoxycarbonyl and aminoalkyl, provided that the optionally substituted heterocycloalkyl group cannot be substituted with more than one group selected from —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with alkyl, (CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, and alkoxycarbonyl; and

R⁶, R⁷, R⁸, R⁹, and R¹⁰ are as defined in above.

Another embodiment relates to a compound of Formula I or IA, or a pharmaceutically acceptable salt thereof, wherein:

R¹ is —NR⁴R⁵, —(C₁-C₄)alky-NR⁸R⁹, or —O—(C₁-C₄)alky-NR⁸R⁹; and

R⁴, R⁵, Wand R⁹ are as defined above 5.

Another embodiment relates to a compound of Formula I or IA, or a pharmaceutically acceptable salt thereof, wherein:

R¹ is —NR⁴R⁵, wherein R⁴ and R⁵ are as defined in any of the embodiments above.

Another embodiment relates to a compound of Formula I or IA, or a pharmaceutically acceptable salt thereof, wherein:

R¹ is a heterocycloalkyl group, wherein the heterocycloalkyl group is selected from morpholinyl, piperidinyl, piperazinyl, pyrrolidinyl, imidazolidinyl, 3-azabicyclo[3.1.0]hexyl, 1,2,3,6-tetrahydropyridinyl, hexahydrocyclopenta[c]pyrrol-2(1H)-yl, hexahydrocyclopenta[c]pyrrol-5(1H)-yl, hexahydropyrrolo[3,4-c]pyrrol-2(1H)-yl), (4aR,8aR)-octahydro-1H-pyrido[3,4-b][1,4]oxazinyl, hexahydropyrrolo[3,4-b]pyrrol-2(1H)-yl), hexahydropyrrolo[3,4-b]pyrrol-5(1H)-yl), thiazolidinyl, tetrahydro-3aH[1,3]dioxolo[4,5-c]pyrrolyl, hexahydropyrrolo[1,2-a]pyrazin-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from halo, alkyl optionally substituted with 1-3 halo, carboxyalkyl, amino, hydroxyl, hydroxyalkyl, alkoxy optionally substituted with 1-3 halo, alkoxyalkyl optionally substituted with 1-3 halo, —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, (C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with NR⁸R⁹, —(CH₂)₀₋₃—N(R⁶)R⁷, —C(O)—N(R⁸)R⁹, alkyl-C(O)N(H)(alkyl)-, aryl, arylalkyl optionally substituted with 1 or 2 groups sele2cted from halo and alkyl, alkoxycarbonyl and aminoalkyl, provided that the optionally substituted heterocycloalkyl group cannot be substituted with more than one group selected from —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with alkyl, (CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, and alkoxycarbonyl; and

R⁶, R⁷, R⁸, R⁹, and R¹⁰ are as defined above.

Another embodiment relates to a compound of Formula I or IA, or a pharmaceutically acceptable salt thereof, wherein:

R¹ is selected from NR⁴R⁵, —(C₁-C₄)alky-NR⁸R⁹, —O—(C₁-C₄)alky-NR⁸R⁹, triazolyl optionally substituted with phenylmethyl, 1H-pyrrolo[2,3-b]pyridinyl, 1H-indolyl, pyridinyl optionally substituted with amino or methoxy, pyrazolyl, pyrimidinyl optionally substituted with amino, (5-6 membered)heterocycloalkyl optionally substituted with hydroxyl, and amino;

R³ is phenyl, pyridine or pyrimidine, wherein the phenyl, pyridine or pyrimidine is optionally substituted with 1 or 2 substituents independently selected from amine, alkoxy, hydroxyl, alkyl and aminocarbonyl;

R⁴ is selected from H, methyl, ethyl and propyl;

R⁵ is selected from is selected from propyl, 1-methylethyl, methyl, ethyl, propyl, 2-methylpropyl, phenylethyl, dimethylaminoethyl, methoxymethyl, piperidinyl optionally substituted with methyl, cyclopentyl, dimethylaminocarbonylmethyl, ethylpyrrolidinylmethyl, piperidinylethyl, methylaminoethyl, aminoethyl, pyrrolidinylethyl, 2-propane-1,3 diol, phenyl, carboxymethyl, dimethylaminopropyl, dimethylaminopropan-2-yl, dimethylaminopropan-1-ol-2-yl, methylaminopropyl, aminocyclohexyl, methoxyethyl, (2R)-pyrrolidinylmethyl, (2S)-pyrrolidinylmethyl, dimethylaminoethyl, pyrrolidinyl optionally substituted with methyl, propane-1,3-2diol, hydroxymethyl, hydroxyethyl, aminomethyl, dimethylaminomethylcarbonylaminoethyl, dimethylamino, (R)-benzyl 2-amino-3-hydroxypropylcarbamate and methoxymethyl;

or R⁴ and R⁵, together with the nitrogen to which they are both attached, join together to form a heteroaryl group or a heterocycloalkyl group, wherein the heteroaryl group is selected from (1) isoindolinyl, (2) 1,2,3,4-tetrahydroisoquinolinyl optionally substituted with hydroxyl, and (3) indolinyl optionally substituted with hydroxyalkyl, and wherein the heterocycloalkyl group is selected from morpholinyl, piperidinyl, piperazinyl, pyrrolidinyl, imidazolidinyl, 3-azabicyclo[3.1.0]hexyl, 1,2,3,6-tetrahydropyridinyl, hexahydrocyclopenta[c]pyrrol-2(1H)-yl, hexahydrocyclopenta[c]pyrrol-5(1H)-yl, hexahydropyrrolo[3,4-c]pyrrol-2(1H)-yl), (4aR,8aR)-octahydro-1H-pyrido[3,4-b][1,4]oxazinyl, hexahydropyrrolo[3,4-b]pyrrol-2(1H)-yl), hexahydropyrrolo[3,4-b]pyrrol-5(1H)-yl), thiazolidinyl, tetrahydro-3aH[1,3]dioxolo[4,5-c]pyrrolyl, hexahydropyrrolo[1,2-a]pyrazin-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from halo, alkyl optionally substituted with 1-3 halo, carboxyalkyl, amino, hydroxyl, hydroxyalkyl, alkoxy optionally substituted with 1-3 halo, alkoxyalkyl optionally substituted with 1-3 halo, —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, (C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with NR⁸R⁹, —(CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, alkoxycarbonyl and aminoalkyl, provided that the optionally substituted heterocycloalkyl group is substituted cannot be substituted with more than one group selected from —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with alkyl, (CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, and alkoxycarbonyl; and

R⁶, R⁷, R⁸, R⁹, and R¹⁰ are as defined in above.

Another embodiment relates to a compound of Formula I or IA, or a pharmaceutically acceptable salt thereof, wherein:

R¹ is a heterocycloalkyl group, wherein the heterocycloalkyl group is selected from morpholinyl, piperidinyl, piperazinyl, pyrrolidinyl, imidazolidinyl, 3-azabicyclo[3.1.0]hexyl, 1,2,3,6-tetrahydropyridinyl, hexahydrocyclopenta[c]pyrrol-2(1H)-yl, hexahydrocyclopenta[c]pyrrol-5(1H)-yl, hexahydropyrrolo[3,4-c]pyrrol-2(1H)-yl), (4aR,8aR)-octahydro-1H-pyrido[3,4-b][1,4]oxazinyl, hexahydropyrrolo[3,4-b]pyrrol-2(1H)-yl), hexahydropyrrolo[3,4-b]pyrrol-5(1H)-yl), thiazolidinyl, tetrahydro-3aH[1,3]dioxolo[4,5-c]pyrrolyl, hexahydropyrrolo[1,2-a]pyrazin-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from halo, alkyl optionally substituted with 1-3 halo, carboxyalkyl, amino, hydroxyl, hydroxyalkyl, alkoxy optionally substituted with 1-3 halo, alkoxyalkyl optionally substituted with 1-3 halo, —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, (C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with NR⁸R⁹, —(CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, alkoxycarbonyl and aminoalkyl, provided that the optionally substituted heterocycloalkyl group cannot be substituted with more than one group selected from —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with alkyl, (CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, and alkoxycarbonyl; and

R⁶, R⁷, R⁸, R⁹, and R¹⁰ are as defined above.

Other embodiments relate to a compound of Formula I, or a pharmaceutically acceptable salt thereof, having Formula II, III, IV, V, VI or VII:

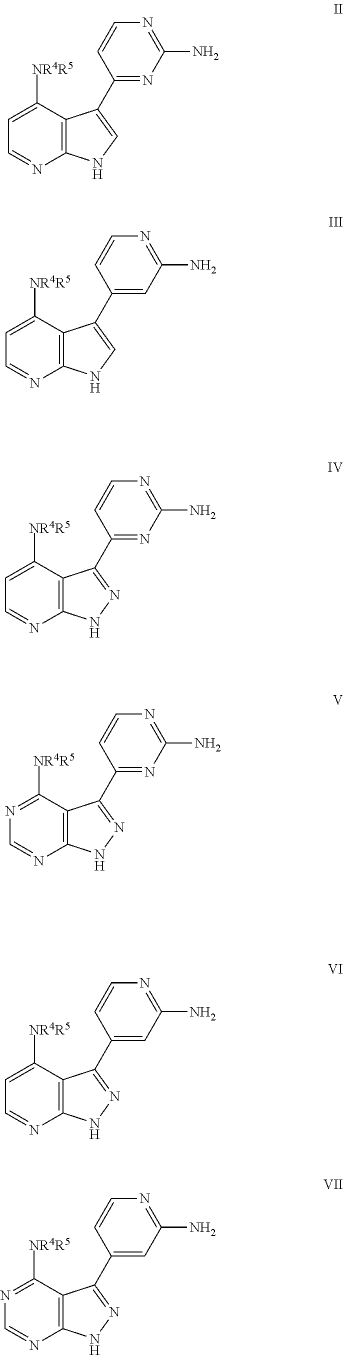

wherein R⁴ and R⁵ are as defined in any of the above embodiments.

When any of the embodiments in this specification refers to a compound of Formula (II), (III, (IV), (V), (VI) or (VII), this is meant to mean that this embodiment includes each of Formula (II), (III, (IV), (V), (VI) or (VII) individually or in any combination of each other. When any of the embodiments in this specification refers to a compound of Formula (I), (II), (III, (IV), (V), (VI) or (VII), this is meant to mean that this embodiment includes each of Formula (I), (II), (III, (IV), (V), (VI) or (VII) individually or in any combination of each other. For instance, when any of the embodiments in this specification refers to a compound of Formula (I), (II), (III, (IV), (V), (VI) or (VII), this is meant to mean that this embodiment includes each of Formula (I), (II), (III, (IV), (V), (VI) or (VII), this can be interpreted to include only compounds having Formula (I), or only compounds having Formula (II), or only compounds having Formula (III), or only compounds having Formula (IV), or only compounds having Formula (V), or only compounds having Formula (VI), or only compounds having Formula (VII), or a combination of any two of Formula (I), (II), (III), (IV), (V), (VI) or (VII), or a combination of any three of Formula (I), (II), (III), (IV), (V), (VI) or (VII), or a combination of any four of Formula (I), (II), (III), (IV), (V), (VI) or (VII), or a combination of any five of Formula (I), (II), (III), (IV), (V), (VI) or (VII), or a combination of any six of Formula (I), (II), (III), (IV), (V), (VI) or (VII), or all of Formula (I), (II), (III), (IV), (V), (VI) and (VII).

In other embodiments of the compounds of Formula (II), (III, (IV), (V), (VI) or (VII), or pharmaceutically acceptable salts thereof,

R⁴ is selected from H, 1-methylethyl, methyl, ethyl, propyl, 2-methylpropyl, phenylethyl, dimethylaminoethyl, methoxymethyl, piperidinyl optionally substituted with methyl, cyclopentyl, dimethylaminocarbonylmethyl, ethylpyrrolidinylmethyl, piperidinylethyl, methylaminoethyl, aminoethyl, pyrrolidinylethyl, 2-propane-1,3 diol, phenyl, carboxymethyl, dimethylaminopropyl, dimethylaminopropan-2-yl, dimethylaminopropan-1-ol-2-yl, methylaminopropyl, aminocyclohexyl, methoxyethyl, (2R)-pyrrolidinylmethyl, (2S)-pyrrolidinylmethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino, (R)-benzyl 2-amino-3-hydroxypropylcarbamate and methoxymethyl;

R⁵ is selected from is selected from propyl, 1-methylethyl, methyl, ethyl, propyl, 2-methylpropyl, phenylethyl, dimethylaminoethyl, methoxymethyl, piperidinyl optionally substituted with methyl, cyclopentyl, dimethylaminocarbonylmethyl, ethylpyrrolidinylmethyl, piperidinylethyl, methylaminoethyl, aminoethyl, pyrrolidinylethyl, 2-propane-1,3 diol, phenyl, carboxymethyl, dimethylaminopropyl, dimethylaminopropan-2-yl, dimethylaminopropan-1-ol-2-yl, methylaminopropyl, aminocyclohexyl, methoxyethyl, (2R)-pyrrolidinylmethyl, (2S)-pyrrolidinylmethyl, dimethylamino-2-ethyl, pyrrolidinyl optionally substituted with methyl, propane-1,3-diol, hydroxymethyl, hydroxyethyl, aminomethyl, dimethylaminomethylcarbonylaminoethyl, dimethylamino, (R)-benzyl 2-amino-3-hydroxypropylcarbamate and methoxymethyl;

or R⁴ and R⁵, together with the nitrogen to which they are both attached, join together to form a heteroaryl group or a heterocycloalkyl group, wherein the heteroaryl group is selected from (1) isoindolinyl, (2) 1,2,3,4-tetrahydroisoquinolinyl optionally substituted with hydroxyl, and (3) indolinyl optionally substituted with hydroxyalkyl, and wherein the heterocycloalkyl group is selected from morpholinyl, piperidinyl, piperazinyl, pyrrolidinyl, imidazolidinyl, 3-azabicyclo[3.1.0]hexyl, 1,2,3,6-tetrahydropyridinyl, hexahydrocyclopenta[c]pyrrol-2(1H)-yl, hexahydrocyclopenta[c]pyrrol-5(1H)-yl, hexahydropyrrolo[3,4-c]pyrrol-2(1H)-yl), (4aR,8aR)-octahydro-1H-pyrido[3,4-b][1,4]oxazinyl, hexahydropyrrolo[3,4-b]pyrrol-2(1H)-yl), hexahydropyrrolo[3,4-b]pyrrol-5(1H)-yl), thiazolidinyl, tetrahydro-3aH[1,3]dioxolo[4,5-c]pyrrolyl, hexahydropyrrolo[1,2-a]pyrazin-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from halo, alkyl optionally substituted with 1-3 halo, carboxyalkyl, amino, hydroxyl, hydroxyalkyl, alkoxy optionally substituted with 1-3 halo, alkoxyalkyl optionally substituted with 1-3 halo, —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, (C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with NR⁸R⁹, —(CH₂)₀₋₃—N(R⁸)R⁷, —C(O)—N(R⁸)R⁹, alkyl-C(O)N(H)(alkyl)-, aryl, arylalkyl optionally substituted with 1 or 2 groups selected from halo and alkyl, alkoxycarbonyl and aminoalkyl, provided that the optionally substituted heterocycloalkyl group cannot be substituted with more than one group selected from —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with alkyl, (CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, and alkoxycarbonyl; and

R⁶, R⁷, R⁸, R⁹, and R¹⁰ are as defined in any of the above embodiments.

In other embodiments of the compounds of Formula (II), (III, (IV), (V), (VI) or (VII), or pharmaceutically acceptable salts thereof,

R⁴ and R⁵, together with the nitrogen to which they are both attached, join together to form a heterocycloalkyl group, wherein the heterocycloalkyl group is selected from morpholinyl, piperidinyl, piperazinyl, pyrrolidinyl, imidazolidinyl, 3-azabicyclo[3.1.0]hexyl, 1,2,3,6-tetrahydropyridinyl, hexahydrocyclopenta[c]pyrrol-2(1H)-yl, hexahydrocyclopenta[c]pyrrol-5(1H)-yl, hexahydropyrrolo[3,4-c]pyrrol-2(1H)-yl), (4aR,8aR)-octahydro-1H-pyrido[3,4-b][1,4]oxazinyl, hexahydropyrrolo[3,4-b]pyrrol-2(1H)-yl), hexahydropyrrolo[3,4-b]pyrrol-5(1H)-yl), thiazolidinyl, tetrahydro-3aH[1,3]dioxolo[4,5-c]pyrrolyl, hexahydropyrrolo[1,2-a]pyrazin-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from halo, alkyl optionally substituted with 1-3 halo, carboxyalkyl, amino, hydroxyl, hydroxyalkyl, alkoxy optionally substituted with 1-3 halo, alkoxyalkyl optionally substituted with 1-3 halo, —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, (C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with NR⁸R⁹, —(CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, alkoxycarbonyl and aminoalkyl, provided that the optionally substituted heterocycloalkyl group cannot be substituted with more than one group selected from —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with alkyl, (CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, and alkoxycarbonyl; and

R⁶, R⁷, R⁸, R⁹, and R¹⁰ are as defined in any of the above embodiments.

The compound of Formula II, III, IV, V, VI or VII according to claim 11, or a pharmaceutically acceptable salt thereof, wherein:

R⁴ and R⁵, together with the nitrogen to which they are both attached, join together to form a heterocycloalkyl group, wherein the heterocycloalkyl group is selected from morpholinyl, piperidinyl, piperazinyl, pyrrolidinyl, imidazolidinyl, 3-azabicyclo[3.1.0]hexyl, 1,2,3,6-tetrahydropyridinyl, hexahydrocyclopenta[c]pyrrol-2(1H)-yl, hexahydrocyclopenta[c]pyrrol-5(1H)-yl, hexahydropyrrolo[3,4-c]pyrrol-2(1H)-yl), (4aR,8aR)-octahydro-1H-pyrido[3,4-b][1,4]oxazinyl, hexahydropyrrolo[3,4-b]pyrrol-2(1H)-yl), hexahydropyrrolo[3,4-b]pyrrol-5(1H)-yl), thiazolidinyl, tetrahydro-3aH[1,3]dioxolo[4,5-c]pyrrolyl, hexahydropyrrolo[1,2-a]pyrazin-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from halo, alkyl optionally substituted with 1-3 halo, carboxyalkyl, amino, hydroxyl, hydroxyalkyl, alkoxy optionally substituted with 1-3 halo, alkoxyalkyl optionally substituted with 1-3 halo, —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, (C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with NR⁸R⁹, —(CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, alkoxycarbonyl and aminoalkyl, provided that the optionally substituted heterocycloalkyl group cannot be substituted with more than one group selected from —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with alkyl, (CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, and alkoxycarbonyl; and

R⁶, R⁷, R⁸, R⁹, and R¹⁰ are as defined in claim 1.

In other embodiments of the compounds of Formula (II), (III, (IV), (V), (VI) or (VII), or pharmaceutically acceptable salts thereof,

R⁴ is H; and

R⁵ is selected from —(C₁-C₃)alkylamino optionally substituted with 1-2 substituents selected from halo, hydroxymethyl and hydroxyl, amino(C₁-C₃)alkylamino, pyrrolidinyl(C₁-C₄)alkylamino, pyridinyll(C₁-C₃)alkylamino, —(C₁-C₃)alkylamino(C₁-C₃)alkylamino, —(C₁-C₃)dialkylamino(C₁-C₃)alkylamino optionally substituted with hydroxymethyl, or

R⁴ and R⁵, together with the nitrogen atom to which they are attached, join together to form piperidinyl, morpholinyl and pyrrolidinyl, wherein the piperidinyl, morpholinyl and pyrrolidinyl are optionally substituted with 1, 2 or 3 groups selected from —(C₁-C₃)alkyl optionally substituted with 1-3 substituents selected from halo and hydroxyl, hydroxyl, hydroxyalkyl, alkylamino, methoxy, aminocarbonyl, amine, aminoalkyl, hydroxymethyl, aminomethyl, dimethylamino, dimethylaminoethoxy, and methoxymethyl.

In other embodiments of the compounds of Formula (II), (III, (IV), (V), (VI) or (VII), or pharmaceutically acceptable salts thereof, R⁴ and R⁵, together with the nitrogen atom to which they are attached, join together to form pyrrolidinyl optionally substituted with 1 group selected from (2R)-methoxymethyl, (3R)-amino, (3S)-amino, (3R)-hydroxymethyl, (3S)-hydroxymethyl, (2R)-hydroxymethyl, (2S)-hydroxymethyl, (3R)-hydroxyl, (3S)-hydroxyl, (2R)-hydroxyl, (2S)-hydroxyl, (2R)-aminocarbonyl, (2S)-aminocabonyl, (2R)-methoxy, (2S)-methoxy, (3R)-fluoro, (3S)-fluoro, (3R)-methoxy, (3S)-methoxy, (2R)-fluoro, (2S)-fluoro, fluoroethyl, (2S)-methoxymethyl, (2R)-methoxymethyl, aminomethyl, (3S)-hydroxyethylamino, (3R)-hydroxy and (3S)-hydroxy.

In other embodiments of the compounds of Formula (II), (III, (IV), (V), (VI) or (VII), or pharmaceutically acceptable salts thereof, R⁴ and R⁵ join together with the nitrogen atom to which they are attached to form a group selected from piperidinyl, morpholinyl and pyrrolidinyl, wherein the piperidinyl, morpholinyl and pyrrolidinyl are optionally substituted with 1, 2 or 3 groups selected from hydroxyl, methyl, ethyl, amine, hydroxymethyl, aminomethyl, dimethylamino and methoxymethyl.

In other embodiments of the compounds of Formula (II), (III, (IV), (V), (VI) or (VII), or pharmaceutically acceptable salts thereof, R⁴ and R⁵ join together with the nitrogen atom to which they are attached to form a group selected from piperidinyl, morpholinyl and pyrrolidinyl, wherein the piperidinyl, morpholinyl and pyrrolidinyl are optionally substituted with 1, 2 or 3 groups selected from methyl, ethyl, hydroxyl, amine, hydroxymethyl, aminomethyl, dimethylamino and methoxymethyl.

In other embodiments of the compounds of Formula (II), (III, (IV), (V), (VI) or (VII), or pharmaceutically acceptable salts thereof, R⁴ and R⁵ join together with the nitrogen atom to which they are attached to form a group selected from piperidinyl, morpholinyl and pyrrolidinyl, wherein the piperidinyl, morpholinyl and pyrrolidinyl are optionally substituted with 1, 2 or 3 groups selected from hydroxyl, methyl, ethyl, amine, hydroxymethyl, aminomethyl, dimethylamino and methoxymethyl.

In other embodiments of the compounds of Formula (II), (III, (IV), (V), (VI) or (VII), or pharmaceutically acceptable salts thereof, R⁴ and R⁵ join together with the nitrogen atom to which they are attached to form a piperidinyl optionally substituted with 1 group selected from methyl, ethyl, hydroxyl, amine, hydroxymethyl, aminomethyl, dimethylamino and methoxymethyl.

In other embodiments of the compounds of Formula (II), (III, (IV), (V), (VI) or (VII), or pharmaceutically acceptable salts thereof, R⁴ and R⁵ join together with the nitrogen atom to which they are attached to form a piperidinyl optionally substituted with 1 group selected from hydroxyl, methyl ethyl, amine, hydroxymethyl, aminomethyl, dimethylamino and methoxymethyl.

In other embodiments of the compounds of Formula (II), (III, (IV), (V), (VI) or (VII), or pharmaceutically acceptable salts thereof, R⁴ and R⁵ join together with the nitrogen atom to which they are attached to form a morpholinyl optionally substituted with 1, group selected from methyl, hydroxyl, methyl, ethyl, amine, hydroxymethyl, aminomethyl, dimethylamino and methoxymethyl.

In other embodiments of the compounds of Formula (II), (III, (IV), (V), (VI) or (VII), or pharmaceutically acceptable salts thereof, R⁴ and R⁵ join together with the nitrogen atom to which they are attached to form a morpholinyl optionally substituted with 1 group selected from hydroxyl, methyl, ethyl, amine, hydroxymethyl, aminomethyl, dimethylamino and methoxymethyl.

In other embodiments of the compounds of Formula (II), (III, (IV), (V), (VI) or (VII), or pharmaceutically acceptable salts thereof, R⁴ and R⁵ join together with the nitrogen atom to which they are attached to form a pyrrolidinyl optionally substituted with 1 group selected from methyl, ethyl, hydroxyl, amine, hydroxymethyl, aminomethyl, dimethylamino and methoxymethyl.

In other embodiments of the compounds of Formula (II), (III, (IV), (V), (VI) or (VII), or pharmaceutically acceptable salts thereof, R⁴ and R⁵ join together with the nitrogen atom to which they are attached to form a pyrrolidinyl optionally substituted with 1 group selected from methyl, ethyl, hydroxyl, amine, hydroxymethyl, aminomethyl, dimethylamino and methoxymethyl.

In other embodiments of the compounds of Formula (II), (III, (IV), (V), (VI) or (VII), or pharmaceutically acceptable salts thereof, R⁴ and R⁵ join together with the nitrogen atom to which they are attached to form a pyrrolidinyl optionally substituted with 1 group selected from (2R)-methoxymethyl, (3R)-amino, (3S)-amino, (3R)-hydroxymethyl, (3S)-hydroxymethyl, (3R)-hydroxyl, (2S)-methoxymethyl, amino, and (3S)-hydroxy.

In other embodiments of the compounds of Formula (II), (III, (IV), (V), (VI) or (VII), or pharmaceutically acceptable salts thereof, R⁴ and R⁵ join together with the nitrogen atom to which they are attached to form a pyrrolidinyl optionally substituted with 1 group selected from (2R)-methoxymethyl, (3R)-amino, (3S)-amino, (3R)-hydroxymethyl, (3S)-hydroxymethyl, (3R)-hydroxyl, (2S)-methoxymethyl, amino, and (3S)-hydroxy.

Another embodiment relates to a compound of Formula I or IA, or a pharmaceutically acceptable salt thereof, wherein:

R¹ is selected from —(C₁-C₃)alkylamino optionally substituted with 1-2 substituents selected from halo, hydroxymethyl and hydroxyl, amino(C₁-C₃)alkylamino, pyrrolidinyl(C₁-C₄)alkylamino, pyridinyll(C₁-C₃)alkylamino, —(C₁-C₃)alkylamino(C₁-C₃)alkylamino, —(C₁-C₃)dialkylamino(C₁-C₃)alkylamino optionally substituted with hydroxymethyl, piperidinyl, morpholinyl and pyrrolidinyl, wherein the piperidinyl, morpholinyl and pyrrolidinyl are optionally substituted with 1, 2 or 3 groups selected from —(C₁-C₃)alkyl optionally substituted with 1-3 substituents selected from halo and hydroxyl, hydroxyl, hydroxyalkyl, alkylamino, methoxy, aminocarbonyl, amine, aminoalkyl, hydroxymethyl, aminomethyl, dimethylamino, dimethylaminoethoxy, and methoxymethyl.

Another embodiment relates to a compound of Formula I or IA, or a pharmaceutically acceptable salt thereof, wherein:

A is CH;

B is CH; and

R¹ is selected from —(C₁-C₃)alkylamino optionally substituted with 1-2 substituents selected from halo, hydroxymethyl and hydroxyl, amino(C₁-C₃)alkylamino, pyrrolidinyl(C₁-C₄)alkylamino, pyridinyll(C₁-C₃)alkylamino, —(C₁-C₃)alkylamino(C₁-C₃)alkylamino, —(C₁-C₃)dialkylamino(C₁-C₃)alkylamino optionally substituted with hydroxymethyl piperidinyl, morpholinyl and pyrrolidinyl, wherein the piperidinyl, morpholinyl and pyrrolidinyl are optionally substituted with 1, 2 or 3 groups selected from —(C₁-C₃)alkyl optionally substituted with 1-3 substituents selected from halo and hydroxyl, hydroxyl, hydroxyalkyl, alkylamino, methoxy, aminocarbonyl, amine, aminoalkyl, hydroxymethyl, aminomethyl, dimethylamino, dimethylaminoethoxy, and methoxymethyl.

Another embodiment relates to a compound of Formula I or IA, or a pharmaceutically acceptable salt thereof, wherein:

R¹ is selected from piperidinyl, morpholinyl and pyrrolidinyl, wherein the piperidinyl, morpholinyl and pyrrolidinyl are optionally substituted with 1, 2 or 3 groups selected from —(C₁-C₃)alkyl optionally substituted with 1-3 substituents selected from halo and hydroxyl, hydroxyl, hydroxyalkyl, alkylamino, methoxy, aminocarbonyl, amine, aminoalkyl, hydroxymethyl, aminomethyl, dimethylamino, dimethylaminoethoxy, and methoxymethyl.

Another embodiment relates to a compound of Formula I or IA, or a pharmaceutically acceptable salt thereof, wherein:

R¹ is selected from NR⁴R⁵, —(C₁-C₄)alky-NR⁸R⁹, —O—(C₁-C₄)alky-NR⁸R⁹, triazolyl optionally substituted with phenylmethyl, 1H-pyrrolo[2,3-b]pyridinyl, 1H-indolyl, pyridinyl optionally substituted with amino or methoxy, pyrazolyl, pyrimidinyl optionally substituted with amino, (5-6 membered)heterocycloalkyl optionally substituted with hydroxyl, and amino; and

R⁴, R⁵, R⁸ and R⁹ are as defined in any of the embodiments described herein.

Another embodiment relates to a compound of Formula IA, or a pharmaceutically acceptable salt thereof, wherein:

R¹ is piperidinyl optionally substituted with 1 or 2 groups selected from methyl, ethyl, hydroxyl phenylcarbonyl optionally substituted with halo, amine, hydroxymethyl, aminomethyl, hydroxyethyl, isopropyl, dimethylamino and methoxymethyl.

Another embodiment relates to a compound of Formula IA, or a pharmaceutically acceptable salt thereof, wherein:

R¹ is pyrrolidinyl optionally substituted with 1 or 2 groups selected from methoxymethyl, amino, hydroxymethyl, hydroxyl, halo, aminocarbonyl, methoxy, fluoroethyl, aminomethyl and hydroxyethylamino.

Another embodiment relates to a compound of Formula IA, or a pharmaceutically acceptable salt thereof, wherein:

R¹ is piperidinyl optionally substituted with 1 or 2 groups selected from methyl, ethyl, hydroxyl phenylcarbonyl optionally substituted with halo, amine, hydroxymethyl, aminomethyl, hydroxyethyl, isopropyl, dimethylamino and methoxymethyl.

Another embodiment relates to a compound of Formula IA, or a pharmaceutically acceptable salt thereof, wherein:

R¹ is pyrrolidinyl optionally substituted with 1 group selected from (2R)-methoxymethyl, (3R)-amino, (3S)-amino, (3R)-hydroxymethyl, (3S)-hydroxymethyl, (2R)-hydroxymethyl, (2S)-hydroxymethyl, (3R)-hydroxyl, (3S)-hydroxyl, (2R)-hydroxyl, (2S)-hydroxyl, (2R)-aminocarbonyl, (2S)-aminocarbonyl, (2R)-methoxy, (2S)-methoxy, (3R)-fluoro, (3S)-fluoro, (3R)-methoxy, (3S)-methoxy, (2R)-fluoro, (2S)-fluoro, fluoroethyl, (2S)-methoxymethyl, (2R)-methoxymethyl, aminomethyl, (3S)-hydroxyethylamino, (3R)-hydroxy and (3S)-hydroxy.

Another embodiment relates to a compound of Formula IA, or a pharmaceutically acceptable salt thereof, wherein:

A is CH;

B is CH; and

R¹ is selected from piperidinyl, morpholinyl and pyrrolidinyl, wherein the piperidinyl, morpholinyl and pyrrolidinyl are optionally substituted with 1, 2 or 3 groups selected from —(C₁-C₃)alkyl optionally substituted with 1-3 substituents selected from halo and hydroxyl, hydroxyl, hydroxyalkyl, alkylamino, methoxy, aminocarbonyl, amine, aminoalkyl, hydroxymethyl, aminomethyl, dimethylamino, dimethylaminoethoxy, and methoxymethyl.

Another embodiment relates to a compound of Formula IA, or a pharmaceutically acceptable salt thereof, wherein:

A is CH;

B is CH; and

R¹ is piperidinyl optionally substituted with 1 or 2 groups selected from methyl, ethyl, hydroxyl phenylcarbonyl optionally substituted with halo, amine, hydroxymethyl, aminomethyl, hydroxyethyl, isopropyl, dimethylamino and methoxymethyl.

Another embodiment relates to a compound of Formula IA, or a pharmaceutically acceptable salt thereof, wherein:

A is CH;

B is CH; and

R¹ is pyrrolidinyl optionally substituted with 1 or 2 groups selected from methoxymethyl, amino, hydroxymethyl, hydroxyl, halo, aminocarbonyl, methoxy, fluoroethyl, aminomethyl and hydroxyethylamino.

Another embodiment relates to a compound of Formula IA or a pharmaceutically acceptable salt thereof, wherein:

A is CH;

B is CH; and

R¹ is piperidinyl optionally substituted with 1 or 2 groups selected from methyl, ethyl, hydroxyl phenylcarbonyl optionally substituted with halo, amine, hydroxymethyl, aminomethyl, hydroxyethyl, isopropyl, dimethylamino and methoxymethyl.

Another embodiment relates to a compound of Formula IA, or a pharmaceutically acceptable salt thereof, wherein:

A is CH;

B is CH; and

R¹ is pyrrolidinyl optionally substituted with 1 group selected from (2R)-methoxymethyl, (3R)-amino, (3S)-amino, (3R)-hydroxymethyl, (3S)-hydroxymethyl, (2R)-hydroxymethyl, (2S)-hydroxymethyl, (3R)-hydroxyl, (3S)-hydroxyl, (2R)-hydroxyl, (2S)-hydroxyl, (2R)-aminocarbonyl, (2S)-aminocarbonyl, (2R)-methoxy, (2S)-methoxy, (3R)-fluoro, (3S)-fluoro, (3R)-methoxy, (3S)-methoxy, (2R)-fluoro, (2S)-fluoro, fluoroethyl, (2S)-methoxymethyl, (2R)-methoxymethyl, aminomethyl, (3S)-hydroxyethylamino, (3R)-hydroxy and (3S)-hydroxy.

Another embodiment relates to a compound of Formula IA, or a pharmaceutically acceptable salt thereof, wherein:

R¹ is selected from —(C₁-C₃)alkylamino optionally substituted with 1-2 substituents selected from halo, hydroxymethyl and hydroxyl, amino(C₁-C₃)alkylamino, pyrrolidinyl(C₁-C₄)alkylamino, pyridinyll(C₁-C₃)alkylamino, —(C₁-C₃)alkylamino(C₁-C₃)alkylamino, and —(C₁-C₃)dialkylamino(C₁-C₃)alkylamino optionally substituted with hydroxymethyl.

Another embodiment relates to a compound of Formula IA, or a pharmaceutically acceptable salt thereof, wherein:

A is CH;

B is CH; and

R¹ is selected from (C₁-C₃)alkylamino optionally substituted with 1-2 substituents selected from halo, hydroxymethyl and hydroxyl, amino(C₁-C₃)alkylamino, pyrrolidinyl(C₁-C₄)alkylamino, pyridinyll(C₁-C₃)alkylamino, (C₁-C₃)alkylamino(C₁-C₃)alkylamino, and (C₁-C₃)dialkylamino(C₁-C₃)alkylamino optionally substituted with hydroxymethyl.

Another embodiment relates to a compound of Formula IB, wherein

R^{Xa} is pyridinyl, 1H-isoindole, halo, phenyl optionally substituted with amine, hydroxyalkyl, aminocarbony, dialkylaminocarbonyl, —S(O)₂—NH₂, and alkylcarbonylamino;

R^{Xb} is selected from (1) pyridine optionally substituted with halo or amine, (2) pyrimidiny optionally substituted with amine or dialkylaminoalkylcarbonylaminoalkylamino, (3) phenyl optionally substituted with —S(O)₂—N(H)—(C₁-C₃)alkyl-OH, —S(O)₂—N(H)—(C₁-C₃)alkyl-phenyl, or hydroxyallyl, (4) 1H-pyrazolo[3,4-b]pyridine, imidazolyl, —S(O)₂—N(H)—(C₁-C₃)alkyl-OH,- and imidazolyl.

In another embodiment of the compound of Formula I or IA, or a pharmaceutically acceptable salt thereof, A is N; and B is CH.

In another embodiment of the compound of Formula I or IA, or a pharmaceutically acceptable salt thereof, A is CH; and B is N.

In another embodiment of the compound of Formula I or IA, or a pharmaceutically acceptable salt thereof, A is CH; and B is CH.

In another embodiment of the compound of Formula I or IA, or a pharmaceutically acceptable salt thereof, A is N; and B is N.

In another embodiment of the compound of Formula I or IA, or a pharmaceutically acceptable salt thereof, R¹ is H; A is CR²; and R² is selected from —NR⁴R⁵, amino and phenyl, wherein R⁴ and R⁵ are as defined in any of the embodiments disclosed in this specification.

In another embodiment of the compound of Formula I or IA, or a pharmaceutically acceptable salt thereof, R¹ is H; A is CR²; and R² is —NR⁴R⁵, wherein R⁴ and R⁵ are as defined in any of the embodiments disclosed in this specification.

In another embodiment of the compound of Formula I or IA, or a pharmaceutically acceptable salt thereof, R¹ is H; A is CR²; and R² is amino.

In another embodiment of the compound of Formula I or IA, or a pharmaceutically acceptable salt thereof, R¹ is H; A is CR²; and R² is phenyl.

In another embodiment of the compound of Formula I or IA, or a pharmaceutically acceptable salt thereof, A is CH; and R¹ is selected from —NR⁴R⁵, amino and phenyl, wherein

—NR⁴R⁵ are as defined in any of the embodiments disclosed in ths specification.

In another embodiment of the compound of Formula I or IA, or a pharmaceutically acceptable salt thereof, A is CH; and R¹ is halo.

In another embodiment of the compound of Formula I or IA, or a pharmaceutically acceptable salt thereof, A is CH; and R¹ is —NR⁴R⁵, wherein R⁴ and R⁵ are as defined in any of the embodiments disclosed in this specification.

In another embodiment of the compound of Formula I or IA, or a pharmaceutically acceptable salt thereof, A is CH; and R¹ is amino.

In another embodiment of the compound of Formula I or IA, or a pharmaceutically acceptable salt thereof, A is CH; and R¹ is phenyl.

In another embodiment of the compound of Formula I or IA, or a pharmaceutically acceptable salt thereof, A is CH; and R¹ is a heterocycloalkyl group optionally substituted with 1, 2 or 3 substituents independently selected from alkyl, amino, hydroxyl, hydroxyalkyl, alkoxyalkyl, alkylcarbonylamino, alkylamino, dialkylamino, alkyl-C(O)N(H)(alkyl)-, aryl, alkoxycarbonyl and aminoalkyl, provided that the heterocycloalkyl group is substituted with no more than one alkylcarbonylamino, alkyl-C(O)N(H)(alkyl)-, aryl or alkoxycarbonyl.

In another embodiment of the compound of Formula I or IA, or a pharmaceutically acceptable salt thereof, R¹ is H; A is CR²; and R² is a heterocycloalkyl group optionally substituted with 1, 2 or 3 substituents independently selected from alkyl, amino, hydroxyl, hydroxyalkyl, alkoxyalkyl, alkylcarbonylamino, alkylamino, dialkylamino, alkyl-C(O)N(H)(alkyl)-, aryl, alkoxycarbonyl and aminoalkyl, provided that the heterocycloalkyl group is substituted with no more than one alkylcarbonylamino, alkyl-C(O)N(H)(alkyl)-, aryl or alkoxycarbonyl.

In another embodiment of the compound of Formula I or IA, A is CR²; R² is H; and R¹ is selected from piperidinyl, morpholinyl and pyrrolidinyl, wherein the piperidinyl, morpholinyl and pyrrolidinyl are optionally substituted with 1, 2 or 3 groups selected from hydroxyl, methyl, ethyl, amine, hydroxymethyl, aminomethyl, dimethylamino and methoxymethyl.

In another embodiment of the compound of Formula I or IA, R¹ is H; A is CR²; and R² is selected from piperidinyl, morpholinyl and pyrrolidinyl, wherein the piperidinyl, morpholinyl and pyrrolidinyl are optionally substituted with 1, 2 or 3 groups selected from methyl, ethyl, hydroxyl, amine, hydroxymethyl, aminomethyl, dimethylamino and methoxymethyl.

In another embodiment of the compound of Formula I or IA, A is CR²; R² is H; and R¹ is selected from piperidinyl, morpholinyl and pyrrolidinyl, wherein the piperidinyl, morpholinyl and pyrrolidinyl are optionally substituted with 1, 2 or 3 groups selected from hydroxyl, methyl, ethyl, amine, hydroxymethyl, aminomethyl, dimethylamino and methoxymethyl.

In another embodiment of the compound of Formula I or IA, R¹ is H; A is CR²; and R² is piperidinyl optionally substituted with 1 group selected from methyl, ethyl, hydroxyl, amine, hydroxymethyl, aminomethyl, dimethylamino and methoxymethyl.

In another embodiment of the compound of Formula I or IA, A is CH; and R¹ is piperidinyl optionally substituted with 1 group selected from hydroxyl, methyl ethyl, amine, hydroxymethyl, aminomethyl, dimethylamino and methoxymethyl.

In another embodiment of the compound of Formula I or IA, R¹ is H; A is CR²; and R² is morpholinyl optionally substituted with 1, group selected from methyl, hydroxyl, methyl, ethyl, amine, hydroxymethyl, aminomethyl, dimethylamino and methoxymethyl.

In another embodiment of the compound of Formula I or IA, A is CH; and R¹ is morpholinyl optionally substituted with 1 group selected from hydroxyl, methyl, ethyl, amine, hydroxymethyl, aminomethyl, dimethylamino and methoxymethyl.

In another embodiment of the compound of Formula I or IA, R¹ is H; and R² is pyrrolidinyl optionally substituted with 1 group selected from methyl, ethyl, hydroxyl, amine, hydroxymethyl, aminomethyl, dimethylamino and methoxymethyl.

In another embodiment of the compound of Formula I or IA, A is CH; and R¹ is pyrrolidinyl optionally substituted with 1 group selected from methyl, ethyl, hydroxyl, amine, hydroxymethyl, aminomethyl, dimethylamino and methoxymethyl.

In another embodiment of the compound of Formula I or IA, R¹ is H; A is CR²; and R² is pyrrolidinyl optionally substituted with 1 group selected from (2R)-methoxymethyl, (3R)-amino, (2S)-amino, (3R)-hydroxymethyl, (3S)-hydroxymethyl, (3R)-hydroxyl, (2S)-methoxymethyl, amino, and (3S)-hydroxy.

In another embodiment of the compound of Formula I or IA, A is CH; and R¹ is pyrrolidinyl optionally substituted with 1 group selected from (2R)-methoxymethyl, (3R)-amino, (3S)-amino, (3R)-hydroxymethyl, (3S)-hydroxymethyl, (3R)-hydroxyl, (2S)-methoxymethyl, amino, and (3S)-hydroxy.

In another embodiment of the compound of Formula I or IA, or a pharmaceutically acceptable salt thereof, A is CH; and R¹ is a heterocycloalkyl group selected from morpholinyl, piperidinyl, piperazinyl, pyrrolidinyl, hexahydrocyclopenta[c]pyrrol-2(1H)-yl, hexahydropyrrolo[3,4-c]pyrrol-2(1H)-yl), azepanyl, and (1S,4S)-2,5-diazabicyclo[2.2.1]heptanyl, wherein the heterocycloalkyl group is optionally substituted with 1, 2 or 3 substituents independently selected from alkyl, amino, hydroxyl, hydroxyalkyl, alkoxy, alkoxyalkyl, alkylcarbonylamino, alkylamino, dialkylamino, alkyl-C(O)N(H)(alkyl)-, aryl, alkoxycarbonyl and aminoalkyl, provided that the heterocycloalkyl group is substituted with no more than one alkylcarbonylamino, alkyl-C(O)N(H)(alkyl)-, aryl or alkoxycarbonyl.

In another embodiment of the compound of Formula I or IA, or a pharmaceutically acceptable salt thereof, R¹ is H; A is CR²; and R² is a heterocycloalkyl group selected from morpholinyl, piperidinyl, piperazinyl, pyrrolidinyl, hexahydrocyclopenta[c]pyrrol-2(1H)-yl, hexahydropyrrolo[3,4-c]pyrrol-2(1H)-yl), azepanyl, and (1S,4S)-2,5-diazabicyclo[2.2.1]heptanyl, wherein the heterocycloalkyl group is optionally substituted with 1, 2 or 3 substituents independently selected from alkyl, amino, hydroxyl, hydroxyalkyl, alkoxy, alkoxyalkyl, alkylcarbonylamino, alkylamino, dialkylamino, alkyl-C(O)N(H)(alkyl)-, aryl, alkoxycarbonyl and aminoalkyl, provided that the heterocycloalkyl group is substituted with no more than one alkylcarbonylamino alkyl-C(O)N(H)(alkyl)-, aryl or alkoxycarbonyl.

In another embodiment of the compound of Formula I or IA, or a pharmaceutically acceptable salt thereof, A is CH; and R¹ is a heterocycloalkyl group selected from morpholinyl, piperidinyl, piperazinyl, pyrrolidinyl, hexahydrocyclopenta[c]pyrrol-2(1H)-yl, hexahydropyrrolo[3,4-c]pyrrol-2(1H)-yl), azepanyl, and (1S,4S)-2,5-diazabicyclo[2.2.1]heptanyl, wherein the heterocycloalkyl group is optionally substituted with 1, 2 or 3 substituents independently selected from methyl, ethyl, amino, hydroxyl, hydroxymethyl, hydroxyethyl, methoxy, methoxymethyl, methylcarbonylamino, methylamino, dimethylamino, diethylamino, alkylcarbonylamino, methylcarbonylmethylamino, phenyl, ethoxycarbonyl and aminomethyl, provided that the heterocycloalkyl group is substituted with no more than one methylcarbonylamino, methylcarbonylmethylamino, phenyl or ethoxycarbonyl.

In another embodiment of the compound of Formula I or IA, or a pharmaceutically acceptable salt thereof, R¹ is H; A is CR²; and R² is a heterocycloalkyl group selected from morpholinyl, piperidinyl, piperazinyl, pyrrolidinyl, hexahydrocyclopenta[c]pyrrol-2(1H)-yl, hexahydropyrrolo[3,4-c]pyrrol-2(1H)-yl), azepanyl, and (1S,4S)-2,5-diazabicyclo[2.2.1]heptanyl,

wherein the heterocycloalkyl group is optionally substituted with 1, 2 or 3 substituents independently selected from methyl, ethyl, amino, hydroxyl, hydroxymethyl, hydroxyethyl, methoxy, methoxymethyl, methylcarbonylamino, methylamino, dimethylamino, diethylamino, alkylcarbonylamino, methylcarbonylmethylamino, phenyl, ethoxycarbonyl and aminomethyl, provided that the heterocycloalkyl group is substituted with no more than one methylcarbonylamino, methylcarbonylmethylamino, phenyl or ethoxycarbonyl.

In another embodiment of the compound of Formula I or IA, or a pharmaceutically acceptable salt thereof, R³ is pyridine or pyrimidine optionally substituted with 1 or 2 substituents independently selected from amine, alkoxy, hydroxyl, alkyl and aminocarbonyl.

In another embodiment of the compound of Formula I or IA, or a pharmaceutically acceptable salt thereof, one of R¹ or R² is H, and the remaining R¹ or R² are each independently selected from H, halo, —NR⁴R⁵, amino and phenyl, wherein R⁴ and R⁵ are as defined in any of the embodiments disclosed in the specification.

In another embodiment of the compound of Formula I or IA, or a pharmaceutically acceptable salt thereof, R³ is pyridine optionally substituted with halo.

In another embodiment of the compound of Formula I or IA, or a pharmaceutically acceptable salt thereof, R³ is pyridine optionally substituted with alkoxy.

In another embodiment of the compound of Formula I or IA, or a pharmaceutically acceptable salt thereof, R³ is pyridine optionally substituted with hydroxyl.

In another embodiment of the compound of Formula I or IA, or a pharmaceutically acceptable salt thereof, R³ is pyridine optionally substituted with methyl.

In another embodiment of the compound of Formula I or IA, or a pharmaceutically acceptable salt thereof, R³ is pyridine optionally substituted with aminocarbonyl.

In another embodiment of the compound of Formula I or IA, or a pharmaceutically acceptable salt thereof, R³ is pyridine optionally substituted with amine.

In another embodiment of the compound of Formula I or IA, or a pharmaceutically acceptable salt thereof, R³ is pyridine optionally substituted with amine and alkoxy.

In another embodiment of the compound of Formula I or IA or a pharmaceutically acceptable salt thereof, R³ is pyridine optionally substituted with amine and hydroxyl.

In another embodiment of the compound of Formula I or IA, or a pharmaceutically acceptable salt thereof, R³ is pyridine optionally substituted with amine and methyl.

In another embodiment of the compound of Formula I or IA, or a pharmaceutically acceptable salt thereof, R³ is pyridine optionally substituted with amine and halo.

In another embodiment of the compound of Formula I or IA, or a pharmaceutically acceptable salt thereof, R³ is pyridine optionally substituted with amine and aminocarbonyl.

In another embodiment of the compound of Formula I or IA or a pharmaceutically acceptable salt thereof, R³ is pyridine or pyrimidine optionally substituted with amine.

In another embodiment of the compound of Formula I or IA, or a pharmaceutically acceptable salt thereof, R³ is pyrimidine optionally substituted with amine.

In another embodiment of the compound of Formula I or IA, or a pharmaceutically acceptable salt thereof, R³ is pyrimidine optionally substituted with amine.

In another embodiment of the compound of Formula I or IA, or a pharmaceutically acceptable salt thereof, R³ is pyrimidine optionally substituted with halo.

In another embodiment of the compound of Formula I or IA, or a pharmaceutically acceptable salt thereof, R³ is pyrimidine optionally substituted with alkoxy.

In another embodiment of the compound of Formula I or IA, or a pharmaceutically acceptable salt thereof, R³ is pyrimidine optionally substituted with hydroxyl.

In another embodiment of the compound of Formula I or IA, or a pharmaceutically acceptable salt thereof, R³ is pyrimidine optionally substituted with methyl.

In another embodiment of the compound of Formula I or IA, or a pharmaceutically acceptable salt thereof, R³ is pyrimidine optionally substituted with aminocarbonyl.

In another embodiment of the compound of Formula I or IA, or a pharmaceutically acceptable salt thereof, R³ is pyrimidine optionally substituted with amine.

In another embodiment of the compound of Formula I or IA, or a pharmaceutically acceptable salt thereof, R³ is pyrimidine optionally substituted with amine and alkoxy.

In another embodiment of the compound of Formula I or IA, or a pharmaceutically acceptable salt thereof, R³ is pyrimidine optionally substituted with amine and hydroxyl.

In another embodiment of the compound of Formula I or IA, or a pharmaceutically acceptable salt thereof, R³ is pyrimidine optionally substituted with amine and methyl.

In another embodiment of the compound of Formula I or IA, or a pharmaceutically acceptable salt thereof, R³ is pyrimidine optionally substituted with amine and halo.

In another embodiment of the compound of Formula I or IA, or a pharmaceutically acceptable salt thereof, R³ is pyrimidine optionally substituted with amine and aminocarbonyl.

In another embodiment of the compound of Formula I or IA, or a pharmaceutically acceptable salt thereof, R³ is

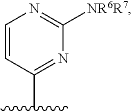

wherein R⁶ and R⁷ are each selected from H and alkyl.

In another embodiment of the compound of Formula I or IA, or a pharmaceutically acceptable salt thereof, R³ is

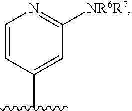

wherein R⁶ and R⁷ are each selected from H and alkyl.

In another embodiment of the compound of Formula I or IA, or a pharmaceutically acceptable salt thereof, R³ is

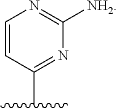

In another embodiment of the compound of Formula I or IA, or a pharmaceutically acceptable salt thereof, R³ is

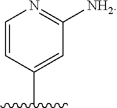

In another embodiment of the compound of Formula I or IA, or a pharmaceutically acceptable salt thereof, R⁴ is selected from H, alkyl, hydroxyalkyl, dihydroxyalkyl, aminoalkyl, dialkylamino, dialkylaminoalkyl, alkoxyalkyl, and heterocycloalkyl optionally substituted with 1, 2 or 3 alkyl; and

R⁵ is selected from alkyl, hydroxyalkyl, dihydroxyalkyl, aminoalkyl, dialkylamino, dialkylaminoalkyl, alkoxyalkyl, and heterocycloalkyl optionally substituted with 1, 2 or 3 alkyl.

In another embodiment of the compound of Formula I or IA, or a pharmaceutically acceptable salt thereof, R⁴ is selected from H, propyl, 1-methylethyl, methyl, ethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino and methoxymethyl; and

R⁵ is selected from propyl, 1-methylethyl, methyl, ethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino and methoxymethyl.

In another embodiment of the compound of Formula I or IA, or a pharmaceutically acceptable salt thereof:

R¹ is —NR⁴R⁵, amino or phenyl;

R³ is phenyl, pyridine or pyrimidine, wherein the phenyl, pyridine or pyrimidine is optionally substituted with 1 or 2 substituents independently selected from amine, alkoxy, hydroxyl, amine, alkyl and aminocarbonyl;

A and B are independently selected from N and CH;

R⁴ is selected from H, alkyl, hydroxyalkyl, dihydroxyalkyl, aminoalkyl, dialkylamino, dialkylaminoalkyl, alkoxyalkyl and heterocycloalkyl optionally substituted with 1, 2 or 3 alkyl; and

R⁵ is selected from alkyl, hydroxyalkyl, dihydroxyalkyl, aminoalkyl, dialkylamino, dialkylaminoalkyl, alkoxyalkyl and heterocycloalkyl optionally substituted with 1, 2 or 3 alkyl;

or R⁴ and R⁵, together with the nitrogen to which they are both attached, join together to form a heterocycloalkyl group optionally substituted with 1, 2 or 3 substituents independently selected from alkyl, amino, hydroxyl, hydroxyalkyl, alkoxy, alkoxyalkyl, alkylcarbonylamino, alkylamino, dialkylamino, alkyl-C(O)N(H)(alkyl)-, aryl, alkoxycarbonyl and aminoalkyl, provided that the heterocycloalkyl group is substituted with n2o more than one alkylcarbonylamino, alkyl-C(O)N(H)(alkyl)-, aryl or alkoxycarbonyl.

In another embodiment of the compound of Formula I or IA, or a pharmaceutically acceptable salt thereof:

R¹ is H;

R² is —NR⁴R⁵, amino or phenyl;

A is CR²;

B is N or CH;

R³ is phenyl, pyridine or pyrimidine, wherein the phenyl, pyridine or pyrimidine is optionally substituted with 1 or 2 substituents independently selected from amine, alkoxy, hydroxyl, amine, alkyl and aminocarbonyl;

R⁴ is selected from H, alkyl, hydroxyalkyl, dihydroxyalkyl, aminoalkyl, dialkylamino, dialkylaminoalkyl, alkoxyalkyl, and heterocycloalkyl optionally substituted with 1, 2 or 3 alkyl; and

R⁵ is selected from alkyl, hydroxyalkyl, dihydroxyalkyl, aminoalkyl, dialkylamino, dialkylaminoalkyl, alkoxyalkyl and heterocycloalkyl optionally substituted with 1, 2 or 3 alkyl;

or R⁴ and R⁵, together with the nitrogen to which they are both attached, join together to form a heterocycloalkyl group optionally substituted with 1, 2 or 3 substituents independently selected from alkyl, amino, hydroxyl, hydroxyalkyl, alkoxy, alkoxyalkyl, alkylcarbonylamino, alkylamino, dialkylamino, alkyl-C(O)N(H)(alkyl)-, aryl, alkoxycarbonyl and aminoalkyl, provided that the heterocycloalkyl group is substituted with no more than one alkylcarbonylamino, alkyl-C(O)N(H)(alkyl)-, aryl or alkoxycarbonyl.

In another embodiment of the compound of Formula I or IA, or a pharmaceutically acceptable salt thereof:

R¹ is —NR⁴R⁵, amino or phenyl;

A is N or CR²;

A and B are independently selected from N and CH;

R² is H when R² is present;

R³ is phenyl, pyridine or pyrimidine, wherein the phenyl, pyridine or pyrimidine is optionally substituted with 1 or 2 substituents independently selected from amine, alkoxy, hydroxyl, amine, alkyl and aminocarbonyl;

R⁴ is selected from H, alkyl, hydroxyalkyl, dihydroxyalkyl, aminoalkyl, dialkylamino, dialkylaminoalkyl, alkoxyalkyl and heterocycloalkyl optionally substituted with 1, 2 or 3 alkyl; and

R⁵ is selected from alkyl, hydroxyalkyl, dihydroxyalkyl, aminoalkyl, dialkylamino, dialkylaminoalkyl, alkoxyalkyl and heterocycloalkyl optionally substituted with 1, 2 or 3 alkyl;

or R⁴ and R⁵, together with the nitrogen to which they are both attached, join together to form a heterocycloalkyl group selected from morpholinyl, piperidinyl, piperazinyl, pyrrolidinyl, hexahydrocyclopenta[c]pyrrol-2(1H)-yl, hexahydropyrrolo[3,4-c]pyrrol-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from alkyl, amino, hydroxyl, hydroxyalkyl, alkoxy, alkoxyalkyl, alkylcarbonylamino, alkylamino, dialkylamino, alkyl-C(O)N(H)(alkyl)-, aryl, alkoxycarbonyl and aminoalkyl, provided that the heterocycloalkyl group is substituted with no more than one alkylcarbonylamino, alkyl-C(O)N(H)(alkyl)-, aryl or alkoxycarbonyl.

In another embodiment of the compound of Formula I or IA, or a pharmaceutically acceptable salt thereof:

R¹ is H;

R² is —NR⁴R⁵, amino or phenyl;

A is CR²;

B is N or CH;

R³ is phenyl, pyridine or pyrimidine, wherein the phenyl, pyridine or pyrimidine is optionally substituted with 1 or 2 substituents independently selected from amine, alkoxy, hydroxyl, amine, alkyl and aminocarbonyl;

R⁴ is selected from H, alkyl, hydroxyalkyl, dihydroxyalkyl, aminoalkyl, dialkylamino, dialkylaminoalkyl, alkoxyalkyl, and heterocycloalkyl optionally substituted with 1, 2 or 3 alkyl; and

R⁵ is selected from alkyl, hydroxyalkyl, dihydroxyalkyl, aminoalkyl, dialkylamino, dialkylaminoalkyl, alkoxyalkyl and heterocycloalkyl optionally substituted with 1, 2 or 3 alkyl;

or R⁴ and R⁵, together with the nitrogen to which they are both attached, join together to form a heterocycloalkyl group selected from morpholinyl, piperidinyl, piperazinyl, pyrrolidinyl, hexahydrocyclopenta[c]pyrrol-2(1H)-yl, hexahydropyrrolo[3,4-c]pyrrol-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from alkyl, amino, hydroxyl, hydroxyalkyl, alkoxy, alkoxyalkyl, alkylcarbonylamino, alkylamino, dialkylamino, alkyl-C(O)N(H)(alkyl)-, aryl, alkoxycarbonyl and aminoalkyl, provided that the heterocycloalkyl group is substituted with no more than one alkylcarbonylamino, alkyl-C(O)N(H)(alkyl)-, aryl or alkoxycarbonyl.

In another embodiment of the compound of Formula I or IA, or a pharmaceutically acceptable salt thereof:

R¹ is H;

R² is —NR⁴R⁵, amino or phenyl;

A is CR²;

B is N or CH;

R³ is phenyl, pyridine or pyrimidine, wherein the phenyl, pyridine or pyrimidine is optionally substituted with 1 or 2 substituents independently selected from amine, alkoxy, hydroxyl, amine, alkyl and aminocarbonyl;

R⁴ is selected from H, alkyl, hydroxyalkyl, dihydroxyalkyl, aminoalkyl, dialkylamino, dialkylaminoalkyl, alkoxyalkyl and heterocycloalkyl optionally substituted with 1, 2 or 3 alkyl; and

R⁵ is selected from alkyl, hydroxyalkyl, dihydroxyalkyl, aminoalkyl, dialkylamino, dialkylaminoalkyl, alkoxyalkyl and heterocycloalkyl optionally substituted with 1, 2 or 3 alkyl;

or R⁴ and R⁵, together with the nitrogen to which they are both attached, join together to form a heterocycloalkyl group selected from morpholinyl, piperidinyl, piperazinyl, pyrrolidinyl, hexahydrocyclopenta[c]pyrrol-2(1H)-yl, hexahydropyrrolo[3,4-c]pyrrol-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from methyl, ethyl, amino, hydroxyl, hydroxymethyl, hydroxyethyl, methoxy, methoxymethyl, methylcarbonylamino, methylamino, dimethylamino, diethylamino, alkylcarbonylamino, methylcarbonylmethylamino, phenyl, ethoxycarbonyl and aminomethyl, provided that the heterocycloalkyl group is substituted with no more than one methylcarbonylamino, methylcarbonylmethylamino, phenyl or ethoxycarbonyl.

In another embodiment of the compound of Formula I or IA, or a pharmaceutically acceptable salt thereof:

R¹ is —NR⁴R⁵, amino or phenyl;

A is CH;

B is N or CH;

R³ is phenyl, pyridine or pyrimidine, wherein the phenyl, pyridine or pyrimidine is optionally substituted with 1 or 2 substituents independently selected from amine, alkoxy, hydroxyl, amine, alkyl and aminocarbonyl;

R⁴ is selected from H, alkyl, hydroxyalkyl, dihydroxyalkyl, aminoalkyl, dialkylamino, dialkylaminoalkyl, alkoxyalkyl, and heterocycloalkyl optionally substituted with 1, 2 or 3 alkyl; and

R⁵ is selected from alkyl, hydroxyalkyl, dihydroxyalkyl, aminoalkyl, dialkylamino, dialkylaminoalkyl, alkoxyalkyl and heterocycloalkyl optionally substituted with 1, 2 or 3 alkyl;

or R⁴ and R⁵, together with the nitrogen to which they are both attached, join together to form a heterocycloalkyl group selected from morpholinyl, piperidinyl, piperazinyl, pyrrolidinyl, hexahydrocyclopenta[c]pyrrol-2(1H)-yl, hexahydropyrrolo[3,4-c]pyrrol-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from methyl, ethyl, amino, hydroxyl, hydroxymethyl, hydroxyethyl, methoxy, methoxymethyl, methylcarbonylamino, methylamino, dimethylamino, diethylamino, alkylcarbonylamino, methylcarbonylmethylamino, phenyl, ethoxycarbonyl and aminomethyl, provided that the heterocycloalkyl group is substituted with no more than one methylcarbonylamino, methylcarbonylmethylamino, phenyl or ethoxycarbonyl.

In another embodiment of the compound of Formula I or IA, or a pharmaceutically acceptable salt thereof:

R¹ is —NR⁴R⁵, amino or phenyl;

R³ is phenyl, pyridine or pyrimidine, wherein the phenyl, pyridine or pyrimidine is optionally substituted with 1 or 2 substituents independently selected from amine, alkoxy, hydroxyl, amine, alkyl and aminocarbonyl;

A is CH;

B is N or CH;

R⁴ is selected from H, propyl, 1-methylethyl, methyl, ethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino and methoxymethyl; and

R⁵ is selected from propyl, 1-methylethyl, methyl, ethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino and methoxymethyl;

or R⁴ and R⁵, together with the nitrogen to which they are both attached, join together to form a heterocycloalkyl group optionally substituted with 1, 2 or 3 substituents independently selected from alkyl, amino, hydroxyl, hydroxyalkyl, alkoxy, alkoxyalkyl, alkylcarbonylamino, alkylamino, dialkylamino, alkyl-C(O)N(H)(alkyl)-, aryl, alkoxycarbonyl and aminoalkyl, provided that the heterocycloalkyl group is substituted with no more than one alkylcarbonylamino, alkyl-C(O)N(H)(alkyl)-, aryl or alkoxycarbonyl.

In another embodiment of the compound of Formula I or IA, or a pharmaceutically acceptable salt thereof:

R¹ is H;

R² is —NR⁴R⁵, amino or phenyl;

A is CR²;

B is N or CH;

R³ is phenyl, pyridine or pyrimidine, wherein the phenyl, pyridine or pyrimidine is optionally substituted with 1 or 2 substituents independently selected from amine, alkoxy, hydroxyl, amine, alkyl and aminocarbonyl;

R⁴ is selected from H, propyl, 1-methylethyl, methyl, ethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino and methoxymethyl; and

R⁵ is selected from propyl, 1-methylethyl, methyl, ethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino and methoxymethyl;

or R⁴ and R⁵, together with the nitrogen to which they are both attached, join together to form a heterocycloalkyl group optionally substituted with 1, 2 or 3 substituents independently selected from alkyl, amino, hydroxyl, hydroxyalkyl, alkoxy, alkoxyalkyl, alkylcarbonylamino, alkylamino, dialkylamino, alkyl-C(O)N(H)(alkyl)-, aryl, alkoxycarbonyl and aminoalkyl, provided that the heterocycloalkyl group is substituted with no more than one alkylcarbonylamino, alkyl-C(O)N(H)(alkyl)-, aryl or alkoxycarbonyl.

In another embodiment of the compound of Formula I or IA, or a pharmaceutically acceptable salt thereof:

R¹ is —NR⁴R⁵, amino or phenyl;

A is CH;

B is N or CH;

R³ is phenyl, pyridine or pyrimidine, wherein the phenyl, pyridine or pyrimidine is optionally substituted with 1 or 2 substituents independently selected from amine, alkoxy, hydroxyl, amine, alkyl and aminocarbonyl;

R⁴ is selected from H, propyl, 1-methylethyl, methyl, ethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino and methoxymethyl; and

R⁵ is selected from propyl, 1-methylethyl, methyl, ethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino and methoxymethyl;

or R⁴ and R⁵, together with the nitrogen to which they are both attached, join together to form a heterocycloalkyl group selected from morpholinyl, piperidinyl, piperazinyl, pyrrolidinyl, hexahydrocyclopenta[c]pyrrol-2(1H)-yl, hexahydropyrrolo[3,4-c]pyrrol-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from alkyl, amino, hydroxyl, hydroxyalkyl, alkoxy, alkoxyalkyl, alkylcarbonylamino, alkylamino, dialkylamino, alkyl-C(O)N(H)(alkyl)-, aryl, alkoxycarbonyl and aminoalkyl, provided that the heterocycloalkyl group is substituted with no more than one alkylcarbonylamino, alkyl-C(O)N(H)(alkyl)-, aryl or alkoxycarbonyl.

In another embodiment of the compound of Formula I or IA, or a pharmaceutically acceptable salt thereof:

R¹ is H;

R² is —NR⁴R⁵, amino or pheny;

A is N or CR²;

B is N or CH;

R³ is phenyl, pyridine or pyrimidine, wherein the phenyl, pyridine or pyrimidine is optionally substituted with 1 or 2 substituents independently selected from amine, alkoxy, hydroxyl, amine, alkyl and aminocarbonyl;

R⁴ is selected from H, propyl, 1-methylethyl, methyl, ethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino and methoxymethyl; and

R⁵ is selected from propyl, 1-methylethyl, methyl, ethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino and methoxymethyl;

or R⁴ and R⁵, together with the nitrogen to which they are both attached, join together to form a heterocycloalkyl group selected from morpholinyl, piperidinyl, piperazinyl, pyrrolidinyl, hexahydrocyclopenta[c]pyrrol-2(1H)-yl, hexahydropyrrolo[3,4-c]pyrrol-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from alkyl, amino, hydroxyl, hydroxyalkyl, alkoxy, alkoxyalkyl, alkylcarbonylamino, alkylamino, dialkylamino, alkyl-C(O)N(H)(alkyl)-, aryl, alkoxycarbonyl and aminoalkyl, provided that the heterocycloalkyl group is substituted with no more than one alkylcarbonylamino, alkyl-C(O)N(H)(alkyl)-, aryl or alkoxycarbonyl.

In another embodiment of the compound of Formula I or IA, or a pharmaceutically acceptable salt thereof:

R¹ is H;

R² is —NR⁴R⁵, amino or phenyl;

A is CR²;

B is N or CH;

R³ is phenyl, pyridine or pyrimidine, wherein the phenyl, pyridine or pyrimidine is optionally substituted with 1 or 2 substituents independently selected from amine, alkoxy, hydroxyl, amine, alkyl and aminocarbonyl;

R⁴ is selected from H, propyl, 1-methylethyl, methyl, ethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino and methoxymethyl; and

R⁵ is selected from propyl, 1-methylethyl, methyl, ethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino and methoxymethyl;

or R⁴ and R⁵, together with the nitrogen to which they are both attached, join together to form a heterocycloalkyl group selected from morpholinyl, piperidinyl, piperazinyl, pyrrolidinyl, hexahydrocyclopenta[c]pyrrol-2(1H)-yl, hexahydropyrrolo[3,4-c]pyrrol-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from alkyl, amino, hydroxyl, hydroxyalkyl, alkoxy, alkoxyalkyl, alkylcarbonylamino, alkylamino, dialkylamino, alkyl-C(O)N(H)(alkyl)-, aryl, alkoxycarbonyl and aminoalkyl, provided that the heterocycloalkyl group is substituted with no more than one alkylcarbonylamino, alkyl-C(O)N(H)(alkyl)-, aryl or alkoxycarbonyl.

In another embodiment of the compound of Formula I or IA, or a pharmaceutically acceptable salt thereof:

R¹ is —NR⁴R⁵, amino or phenyl;

A is CH;

B is N or CH;

R³ is phenyl, pyridine or pyrimidine, wherein the phenyl, pyridine or pyrimidine is optionally substituted with 1 or 2 substituents independently selected from amine, alkoxy, hydroxyl, amine, alkyl and aminocarbonyl;

R⁴ is selected from H, propyl, 1-methylethyl, methyl, ethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino and methoxymethyl; and

R⁵ is selected from propyl, 1-methylethyl, methyl, ethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino and methoxymethyl;

or R⁴ and R⁵, together with the nitrogen to which they are both attached, join together to form a heterocycloalkyl group selected from morpholinyl, piperidinyl, piperazinyl, pyrrolidinyl, hexahydrocyclopenta[c]pyrrol-2(1H)-yl, hexahydropyrrolo[3,4-c]pyrrol-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from alkyl, amino, hydroxyl, hydroxyalkyl, alkoxy, alkoxyalkyl, alkylcarbonylamino, alkylamino, dialkylamino, alkyl-C(O)N(H)(alkyl)-, aryl, alkoxycarbonyl and aminoalkyl.

All compounds of Formula I disclosed above include any of the disclosed alternative aspects or embodiments for each of R¹, R², R³, R⁴, R⁵, R⁶, R⁷, R⁸, R⁹, R¹⁰, A, B, D, R^{xa}, R^{xb} and R^{xc} in combination with any other of the disclosed alternative aspects or embodiments of R¹, R², R³, R⁴, R⁵, R⁶, R⁷, R⁸, R⁹, R¹⁰, A, B, D, R^{xa}, R^{xb} and R^{xc} as well as any pharmaceutically acceptable salt and stereoisomer of any such combination.

All compounds of Formula IA disclosed above include any of the disclosed alternative aspects or embodiments for each of R¹, R², R³, R⁴, R⁵, R⁶, R⁷, R⁸, R⁹, R¹⁰, A and B in combination with any other of the disclosed alternative aspects or embodiments of R¹, R², R³, R⁴, R⁵, R⁶, R⁷, R⁸, R⁹, R¹⁰, A and B as well as any pharmaceutically acceptable salt and stereoisomer of any such combination.

Another aspect of this disclosure relates to a compound of Formula IC:

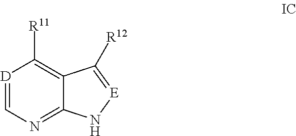

or a pharmaceutically acceptable salt thereof, wherein

R¹⁰ is alkoxy;

R¹¹ is a (5-9 membered)heteroaryl or phenyl, wherein the (5-9 membered)heteroaryl or phenyl are each optionally substituted with 1 or 2 substituents independently selected from amine, alkoxy, hydroxyl, amine, alkyl, aminocarbonyl and halo; and

D and E are independently selected from N and CH, provided that D and E are not both nitrogen.

In another embodiment, the compound of Formula IC is selected from one of the following compounds, or a pharmaceutically acceptable salt thereof:
4-chloro-6-[4-(methyloxy)-1H-pyrrolo[2,3-b]pyridin-3-yl]pyrimidin-2-amine;
4-(methyloxy)-3-[2-(4-methylpiperazin-1-yl)pyridin-4-yl]-1H-pyrrolo[2,3-b]pyridine;
2-fluoro-5-[4-(methyloxy)-1H-pyrrolo[2,3-b]pyridin-3-yl]aniline;
4′-chloro-4-(methyloxy)-1H,1′H-3,5′-bipyrrolo[2,3-b]pyridine;
4-(methyloxy)-3-pyridin-4-yl-1H-pyrazolo[3,4-d]pyrimidine;
4-[4-(methyloxy)-1H-pyrazolo[3,4-d]pyrimidin-3-yl]pyridin-2-amine;
4-(methyloxy)-3-pyridin-3-yl-1H-pyrazolo[3,4-d]pyrimidine;
4-[4-(methyloxy)-7H-pyrrolo[2,3-d]pyrimidin-5-yl]pyridin-2-amine; and
4-[4-(ethyloxy)-1H-pyrrolo[2,3-b]pyridin-3-yl]-6-methylpyrimidin-2-amine.

In another embodiment of the compound of Formula IC, or a pharmaceutically acceptable salt thereof, D is N; and E is CH.

In another embodiment of the compound of Formula IC, or a pharmaceutically acceptable salt thereof, D is CH; and E is N.

In another embodiment of the compound of Formula IC, or a pharmaceutically acceptable salt thereof, A is CH; and B is CH.

In another embodiment of the compound of Formula IC, or a pharmaceutically acceptable salt thereof, A is N; and B is N.

In another embodiment of the compound of Formula IC, or a pharmaceutically acceptable salt thereof, R¹¹ is pyridine optionally substituted with 1 or 2 groups selected from halo and alkyl.

In another embodiment of the compound of Formula IC, or a pharmaceutically acceptable salt thereof, R¹¹ is pyrimidine optionally substituted with 1 or 2 groups selected from halo and alkyl.

In another embodiment of the compound of Formula IC, or a pharmaceutically acceptable salt thereof, R¹¹ is pyrimidine substituted with 1 or 2 groups selected from halo and alkyl.

In another embodiment of the compound of Formula IC, or a pharmaceutically acceptable salt thereof, R¹¹ is pyrrolo[2,3-b]pyridine optionally substituted with 1 or 2 groups selected from halo and alkyl.

All compounds of Formula IC disclosed above include any of the disclosed alternative aspects or embodiments for each of R¹¹, D and E in combination with any other of the disclosed alternative aspects or embodiments of R¹¹, D and E, as well as any pharmaceutically acceptable salt and stereoisomer of any such combination.

Other embodiments include any of the compounds in TABLE IA, TABLE IB and/or TABLE IC that fall within the scope of any of the embodiments described above for the compounds of Formula (I), (IB), (!C), (II), (III, (IV), (V), (VI) or (VII), or pharmaceutically acceptable salts thereof.

TABLE 1A

| Structure | Name |
|---|---|
| 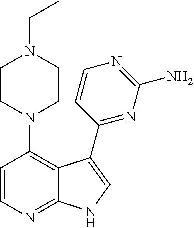 | 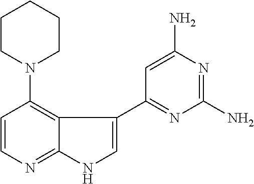 |
| 4-[4-(4-ethylpiperazin-1-yl)-1H- | 6-(4-piperidin-1-yl-1H- |
| pyrrolo[2,3-b]pyridin-3- | pyrrolo[2,3-b]pyridin-3- |
| yl]pyrimidin-2-amine | yl)pyrimidine-2,4-diamine |
| 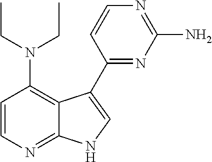 | 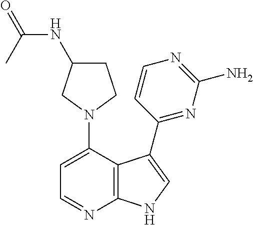 |
| 3-(2-aminopyrimidin-4-yl)-N,N- | N-{1-[3-(2-aminopyrimidin-4-yl)- |
| diethyl-1H-pyrrolo[2,3-b]pyridin-4- | 1H-pyrrolo[2,3-b]pyridin-4- |
| amine | yl]pyrrolidin-3-yl}acetamide |
| 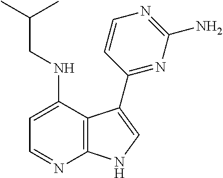 | 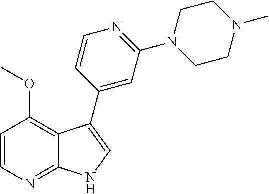 |
| 3-(2-aminopyrimidin-4-yl)-N-(2- | 4-(methyloxy)-3-[2-(4- |
| methylpropyl)-1H-pyrrolo[2,3- | methylpiperazin-1-yl)pyridin-4- |
| b]pyridin-4-amine | yl]-1H-pyrrolo[2,3-b]pyridine |
| 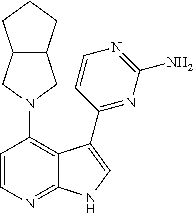 | 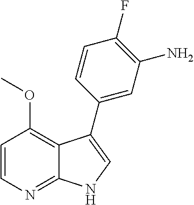 |
| 4-[4-(hexahydrocyclopenta[c]pyrrol- | 2-fluoro-5-[4-(methyloxy)-1H- |
| 2(1H)-yl)-1H-pyrrolo[2,3-b]pyridin- | pyrrolo[2,3-b]pyridin-3-yl]aniline |
| 3-yl]pyrimidin-2-amine | |
| 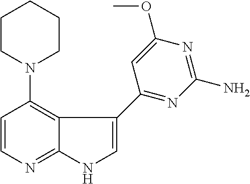 | 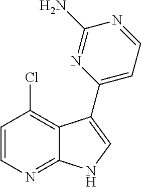 |
| 4-(methyloxy)-6-(4-piperidin-1-yl- | 4-(4-chloro-1H-pyrrolo[2,3- |
| 1H-pyrrolo[2,3-b]pyridin-3- | b]pyridin-3-yl)pyrimidin-2-amine |
| yl)pyrimidin-2-amine | |
| 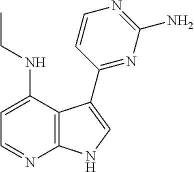 | 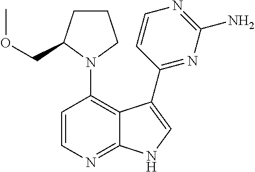 |
| 3-(2-aminopyrimidin-4-yl)-N-ethyl- | 4-(4-{(2R)-2- |
| 1H-pyrrolo[2,3-b]pyridin-4-amine | [(methyloxy)methyl]pyrrolidin-1- |
| | yl}-1H-pyrrolo[2,3-b]pyridin-3- |
| | yl)pyrimidin-2-amine |
| 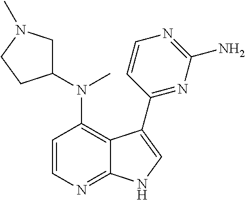 | 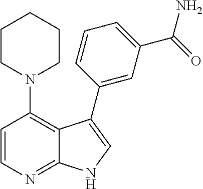 |
| 3-(2-aminopyrimidin-4-yl)-N- | 3-(4-(piperidin-1-yl)-1H- |
| methyl-N-(1-methylpyrrolidin-3-yl)- | pyrrolo[2,3-b]pyridin-3- |
| 1H-pyrrolo[2,3-b]pyridin-4-amine | yl)benzamide |
| 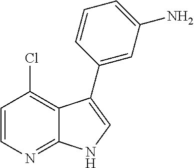 | 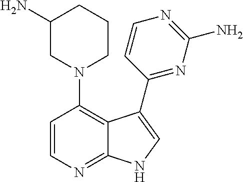 |
| 3-(4-chloro-1H-pyrrolo[2,3- | 4-[4-(3-aminopiperidin-1-yl)-1H- |
| b]pyridin-3-yl)aniline | pyrrolo[2,3-b]pyridin-3- |
| | yl]pyrimidin-2-amine |
| 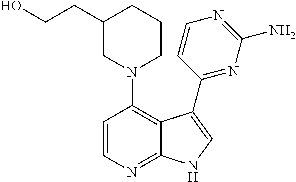 | 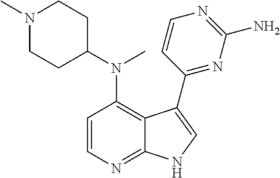 |
| 2-{1-[3-(2-aminopyrimidin-4-yl)- | 3-(2-aminopyrimidin-4-yl)-N- |
| 1H-pyrrolo[2,3-b]pyridin-4- | methyl-N-(1-methylpiperidin-4- |
| yl]piperidin-3-yl}ethanol | yl)-1H-pyrrolo[2,3-b]pyridin-4- |
| | amine |
| 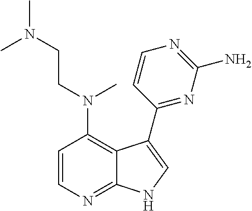 | 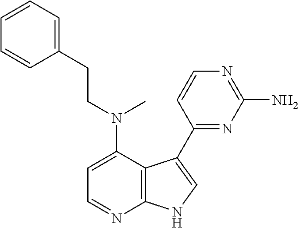 |
| N-[3-(2-aminopyrimidin-4-yl)-1H- | 3-(2-aminopyrimidin-4-yl)-N- |
| pyrrolo[2,3-b]pyridin-4-yl]-N,N′,N′- | methyl-N-(2-phenylethyl)-1H- |
| trimethylethane-1,2-diamine | pyrrolo[2,3-b]pyridin-4-amine |
| 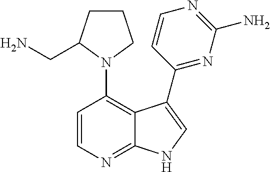 | 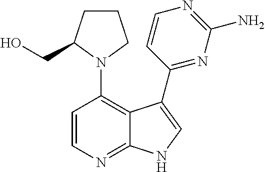 |
| 4-{4-[2-(aminomethyl)pyrrolidin-1- | (R)-(1-(3-(2-aminopyrimidin-4- |
| yl]-1H-pyrrolo[2,3-b]pyridin-3- | yl)-1H-pyrrolo[2,3-b]pyridin-4- |
| yl}pyrimidin-2-amine | yl)pyrrolidin-2-yl)methanol |
| 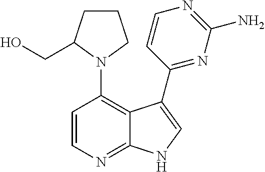 | 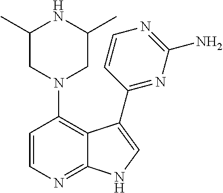 |
| {1-[3-(2-aminopyrimidin-4-yl)-1H- | 4-[4-(3,5-dimethylpiperazin-1-yl)- |
| pyrrolo[2,3-b]pyridin-4- | 1H-pyrrolo[2,3-b]pyridin-3- |
| yl]pyrrolidin-2-yl}methanol | yl]pyrimidin-2-amine |
| 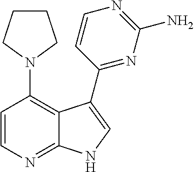 | 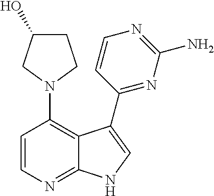 |
| 4-(4-pyrrolidin-1-yl-1H-pyrrolo[2,3- | (3R)-1-[3-(2-aminopyrimidin-4- |
| b]pyridin-3-yl)pyrimidin-2-amine | yl)-1H-pyrrolo[2,3-b]pyridin-4- |
| | yl]pyrrolidin-3-ol |
| 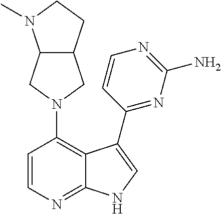 | 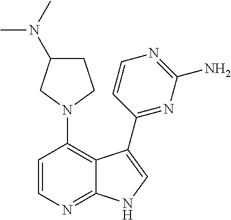 |
| 4-[4-(1- | 4-{4-[3- |
| methylhexahydropyrrolo[3,4- | (dimethylamino)pyrrolidin-1-yl]- |
| b]pyrrol-5(1H)-yl)-1H-pyrrolo[2,3- | 1H-pyrrolo[2,3-b]pyridin-3- |
| b]pyridin-3-yl]pyrimidin-2-amine | yl}pyrimidin-2-amine |
| 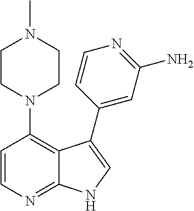 | 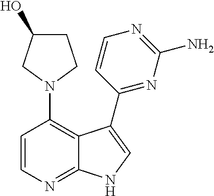 |
| 4-[4-(4-methylpiperazin-1-yl)-1H- | (3S)-1-[3-(2-aminopyrimidin-4- |
| pyrrolo[2,3-b]pyridin-3-yl]pyridin- | yl)-1H-pyrrolo[2,3-b]pyridin-4- |
| 2-amine | yl]pyrrolidin-3-ol |
| 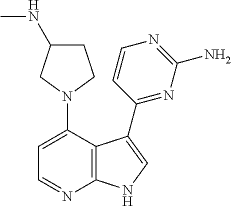 | 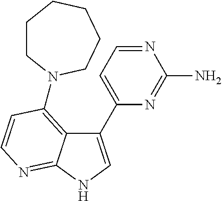 |
| 4-{4-[3-(methylamino)pyrrolidin-1- | 4-(4-azepan-1-yl-1H-pyrrolo[2,3- |
| yl]-1H-pyrrolo[2,3-b]pyridin-3- | b]pyridin-3-yl)pyrimidin-2-amine |
| yl}pyrimidin-2-amine | |
| 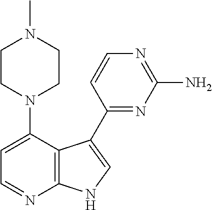 | 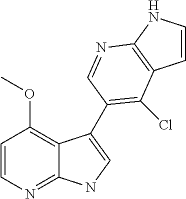 |
| 4-[4-(4-methylpiperazin-1-yl)-1H- | 4′-chloro-4-(methyloxy)-1H,1′H- |
| pyrrolo[2,3-b]pyridin-3- | 3,5′-bipyrrolo[2,3-b]pyridine |
| yl]pyrimidin-2-amine | |
| 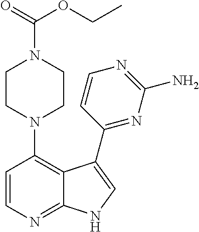 | 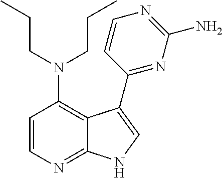 |
| ethyl 4-[3-(2-aminopyrimidin-4-yl)- | 3-(2-aminopyrimidin-4-yl)-N,N- |
| 1H-pyrrolo[2,3-b]pyridin-4- | dipropyl-1H-pyrrolo[2,3- |
| yl]piperazine-1-carboxylate | b]pyridin-4-amine |
| 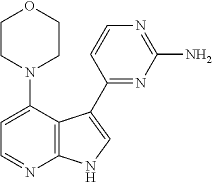 | 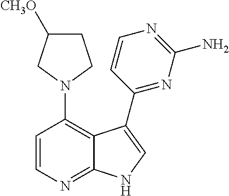 |
| 4-(4-morpholin-4-yl-1H- | 4-{4-[3-(methyloxy)pyrrolidin-1- |
| pyrrolo[2,3-b]pyridin-3- | yl]-1H-pyrrolo[2,3-b]pyridin-3- |
| yl)pyrimidin-2-amine | yl}pyrimidin-2-amine |
| 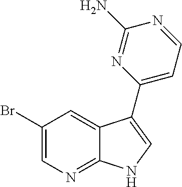 | 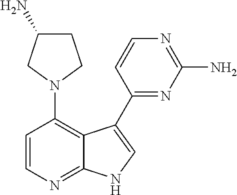 |
| 4-(5-bromo-1H-pyrrolo[2,3- | 4-{4-[(3R)-3-aminopyrrolidin-1- |
| b]pyridin-3-yl)pyrimidin-2-amine | yl]-1H-pyrrolo[2,3-b]pyridin-3- |
| | yl}pyrimidin-2-amine |
| 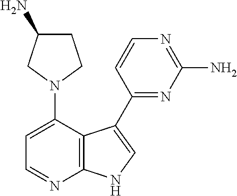 | 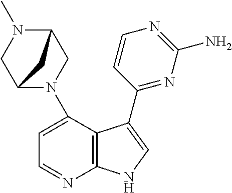 |
| 4-{4-[(3S)-3-aminopyrrolidin-1-yl]- | 4-(4-((1S,4S)-5-methyl-2,5- |
| 1H-pyrrolo[2,3-b]pyridin-3- | diazabicyclo[2.2.1]heptan-2-yl)- |
| yl}pyrimidin-2-amine | 1H-pyrrolo[2,3-b]pyridin-3- |
| | yl)pyrimidin-2-amine |
| 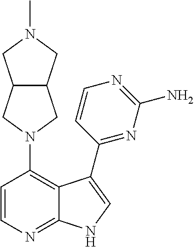 | 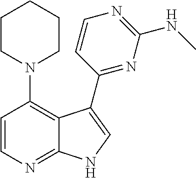 |
| 4-[4-(5- | N-methyl-4-(4-piperidin-1-yl-1H- |
| methylhexahydropyrrolo[3,4- | pyrrolo[2,3-b]pyridin-3- |
| c]pyrrol-2(1H)-yl)-1H-pyrrolo[2,3- | yl)pyrimidin-2-amine |
| b]pyridin-3-yl]pyrimidin-2-amine | |
| 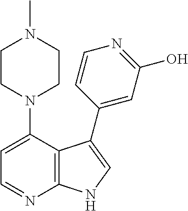 | 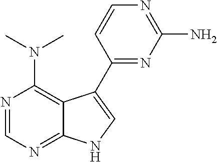 |
| 4-[4-(4-methylpiperazin-1-yl)-1H- | 5-(2-aminopyrimidin-4-yl)-N,N- |
| pyrrolo[2,3-b]pyridin-3-yl]pyridin- | dimethyl-7H-pyrrolo[2,3- |
| 2-ol | d]pyrimidin-4-amine |
| 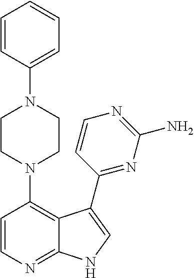 | 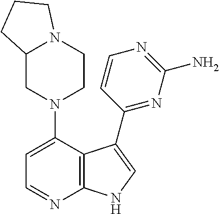 |
| 4-[4-(4-phenylpiperazin-1-yl)-1H- | 4-(4-(hexahydropyrrolo[1,2- |
| pyrrolo[2,3-b]pyridin-3- | a]pyrazin-2(1H)-yl)-1H- |
| yl]pyrimidin-2-amine | pyrrolo[2,3-b]pyridin-3- |
| | yl)pyrimidin-2-amine |
| 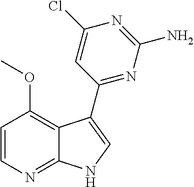 | 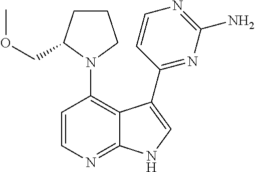 |
| 4-chloro-6-[4-(methyloxy)-1H- | 4-(4-{(2S)-2- |
| pyrrolo[2,3-b]pyridin-3- | [(methyloxy)methyl]pyrrolidin-1- |
| yl]pyrimidin-2-amine | yl}-1H-pyrrolo[2,3-b]pyridin-3- |
| | yl)pyrimidin-2-amine |
| 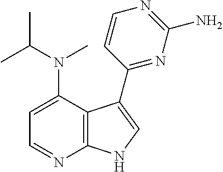 | 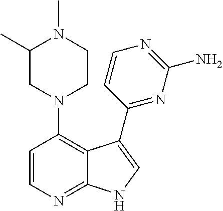 |
| 3-(2-aminopyrimidin-4-yl)-N- | 4-[4-(3,4-dimethylpiperazin-1-yl)- |
| methyl-N-(1-methylethyl)-1H- | 1H-pyrrolo[2,3-b]pyridin-3- |
| pyrrolo[2,3-b]pyridin-4-amine | yl]pyrimidin-2-amine |
| 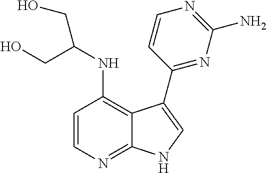 | 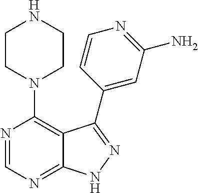 |
| 2-{[3-(2-aminopyrimidin-4-yl)-1H- | 4-(4-piperazin-1-yl-1H- |
| pyrrolo[2,3-b]pyridin-4- | pyrazolo[3,4-d]pyrimidin-3- |
| yl]amino}propane-1,3-diol | yl)pyridin-2-amine |
| 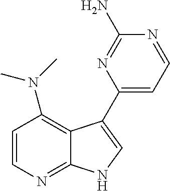 | 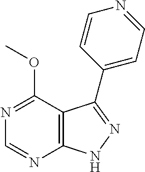 |
| 3-(2-aminopyrimidin-4-yl)-N,N- | 4-(methyloxy)-3-pyridin-4-yl-1H- |
| dimethyl-1H-pyrrolo[2,3-b]pyridin- | pyrazolo[3,4-d]pyrimidine |
| 4-amine | |
| 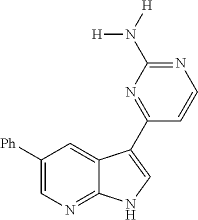 | 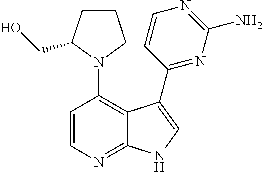 |
| 4-(5-phenyl-1H-pyrrolo[2,3- | {(2S)-1-[3-(2-aminopyrimidin-4- |
| b]pyridin-3-yl)pyrimidin-2-amine | yl)-1H-pyrrolo[2,3-b]pyridin-4- |
| | yl]pyrrolidin-2-yl}methanol |
| 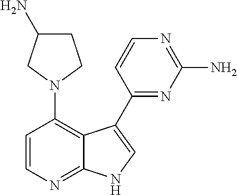 | 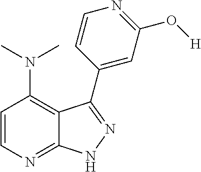 |
| 4-[4-(3-aminopyrrolidin-1-yl)-1H- | 4-[4-(dimethylamino)-1H- |
| pyrrolo[2,3-b]pyridin-3- | pyrazolo[3,4-b]pyridin-3- |
| yl]pyrimidin-2-amine | yl]pyridin-2-ol |
| 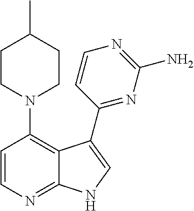 | 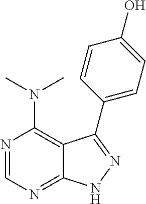 |
| 4-[4-(4-methylpiperidin-1-yl)-1H- | 4-[4-(dimethylamino)-1H- |
| pyrrolo[2,3-b]pyridin-3- | pyrazolo[3,4-d]pyrimidin-3- |
| yl]pyrimidin-2-amine | yl]phenol |
| 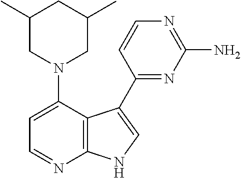 | 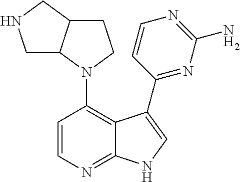 |
| 4-[4-(3,5-dimethylpiperidin-1-yl)- | 4-[4-(hexahydropyrrolo[3,4- |
| 1H-pyrrolo[2,3-b]pyridin-3- | b]pyrrol-1(2H)-yl)-1H- |
| yl]pyrimidin-2-amine | pyrrolo[2,3-b]pyridin-3- |
| | yl]pyrimidin-2-amine |
| 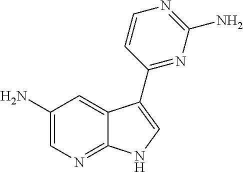 | 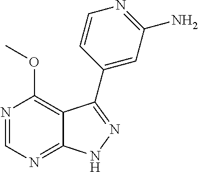 |
| 3-(2-aminopyrimidin-4-yl)-1H- | 4-[4-(methyloxy)-1H- |
| pyrrolo[2,3-b]pyridin-5-amine | pyrazolo[3,4-d]pyrimidin-3- |
| | yl]pyridin-2-amine |
| 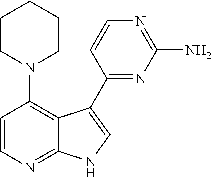 | 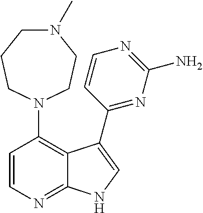 |
| 4-(4-piperidin-1-yl-1H-pyrrolo[2,3- | 4-[4-(4-methyl-1,4-diazepan-1-yl)- |
| b]pyridin-3-yl)pyrimidin-2-amine | 1H-pyrrolo[2,3-b]pyridin-3- |
| | yl]pyrimidin-2-amine |
| 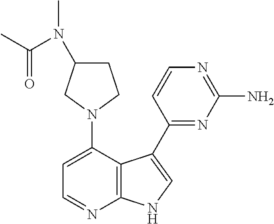 | 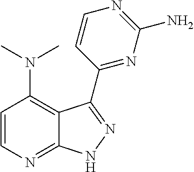 |
| N-{1-[3-(2-aminopyrimidin-4-yl)- | 3-(2-aminopyrimidin-4-yl)-N,N- |
| 1H-pyrrolo[2,3-b]pyridin-4- | dimethyl-1H-pyrazolo[3,4- |
| yl]pyrrolidin-3-yl}-N- | b]pyridin-4-amine |
| methylacetamide | |
| 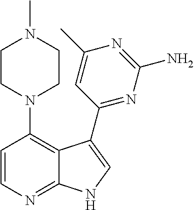 | 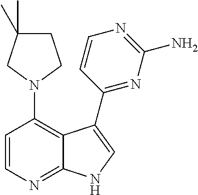 |
| 4-methyl-6-[4-(4-methylpiperazin-1- | 4-[4-(3,3-dimethylpyrrolidin-1-yl)- |
| yl)-1H-pyrrolo[2,3-b]pyridin-3- | 1H-pyrrolo[2,3-b]pyridin-3- |
| yl]pyrimidin-2-amine | yl]pyrimidin-2-amine |
| 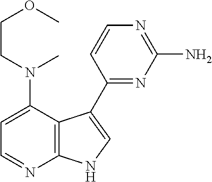 | 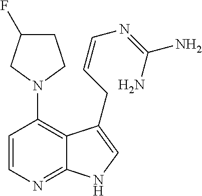 |
| 3-(2-aminopyrimidin-4-yl)-N- | 4-{4-[(3S)-3-fluoropyrrolidin-1- |
| methyl-N-[2-(methyloxy)ethyl]-1H- | yl]-1H-pyrrolo[2,3-b]pyridin-3- |
| pyrrolo[2,3-b]pyridin-4-amine | yl}pyrimidin-2-amine |
| 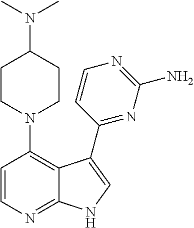 | 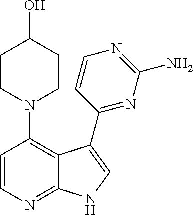 |
| 4-{4-[4-(dimethylamino)piperidin-1- | 1-[3-(2-aminopyrimidin-4-yl)-1H- |
| yl]-1H-pyrrolo[2,3-b]pyridin-3- | pyrrolo[2,3-b]pyridin-4- |
| yl}pyrimidin-2-amine | yl]piperidin-4-ol |
| 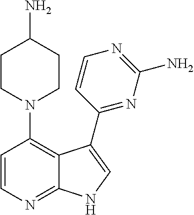 | 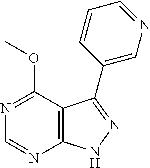 |
| 4-[4-(4-aminopiperidin-1-yl)-1H- | 4-(methyloxy)-3-pyridin-3-yl-1H- |
| pyrrolo[2,3-b]pyridin-3- | pyrazolo[3,4-d]pyrimidine |
| yl]pyrimidin-2-amine | |
| 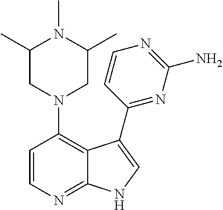 | 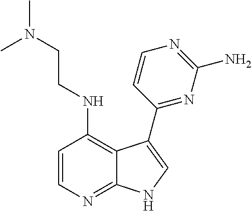 |
| 4-[4-(3,4,5-trimethylpiperazin-1-yl)- | N′-[3-(2-aminopyrimidin-4-yl)- |
| 1H-pyrrolo[2,3-b]pyridin-3- | 1H-pyrrolo[2,3-b]pyridin-4-yl]- |
| yl]pyrimidin-2-amine | N,N-dimethylethane-1,2-diamine |
| 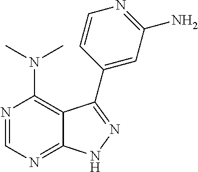 | 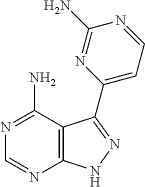 |
| 3-(2-aminopyridin-4-yl)-N,N- | 3-(2-aminopyrimidin-4-yl)-1H- |
| dimethyl-1H-pyrazolo[3,4- | pyrazolo[3,4-d]pyrimidin-4-amine |
| d]pyrimidin-4-amine | |
| 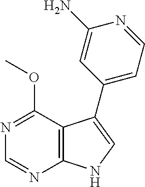 | 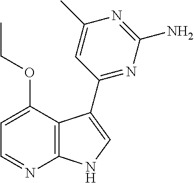 |
| 4-[4-(methyloxy)-7H-pyrrolo[2,3- | 4-[4-(ethyloxy)-1H-pyrrolo[2,3- |
| d]pyrimidin-5-yl]pyridin-2-amine | b]pyridin-3-yl]-6- |
| | methylpyrimidin-2-amine |
| 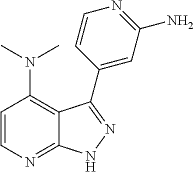 | 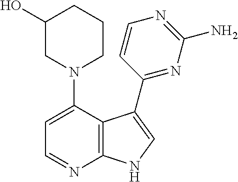 |
| 3-(2-aminopyridin-4-yl)-N,N- | 1-[3-(2-aminopyrimidin-4-yl)-1H- |
| dimethyl-1H-pyrazolo[3,4- | pyrrolo[2,3-b]pyridin-4-yl]piperidin-3-ol |
| b]pyridin-4-amine | |
| 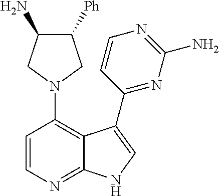 | 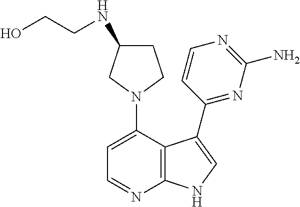 |
| 4-{4-[(3S,4R)-3-amino-4- | 2-({(3S)-1-[3-(2-aminopyrimidin-4-yl)- |
| phenylpyrrolidin-1-yl]-1H-pyrrolo[2,3- | 1H-pyrrolo[2,3-b]pyridin-4- |
| b]pyridin-3-yl}pyrimidin-2-amine | yl]pyrrolidin-3-yl}amino)ethanol |
| 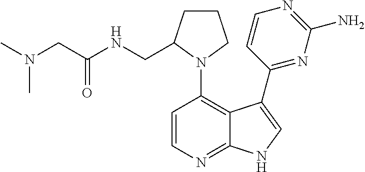 | 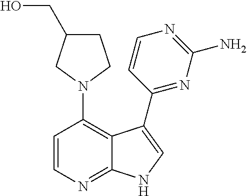 |
| N-({1-[3-(2-aminopyrimidin-4-yl)-1H- | {1-[3-(2-aminopyrimidin-4-yl)-1H- |
| pyrrolo[2,3-b]pyridin-4-yl]pyrrolidin-2- | pyrrolo[2,3-b]pyridin-4-yl]pyrrolidin-3- |
| yl}methyl)-N²,N²-dimethylglycinamide | yl}methanol |
| 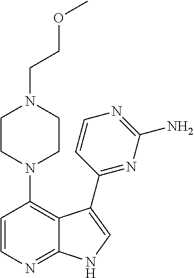 | 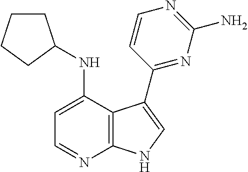 |
| 4-(4-{4-[2-(methyloxy)ethyl]piperazin-1- | 3-(2-aminopyrimidin-4-yl)-N- |
| yl}-1H-pyrrolo[2,3-b]pyridin-3- | cyclopentyl-1H-pyrrolo[2,3-b]pyridin- |
| yl)pyrimidin-2-amine | 4-amine |
| 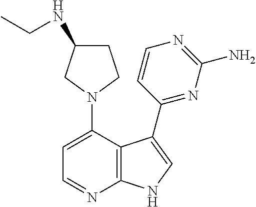 | 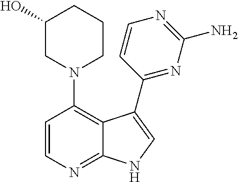 |
| 4-{4-[(3S)-3-(ethylamino)pyrrolidin-1-yl]- | (3R)-1-[3-(2-aminopyrimidin-4-yl)-1H- |
| 1H-pyrrolo[2,3-b]pyridin-3-yl}pyrimidin- | pyrrolo[2,3-b]pyridin-4-yl]piperidin-3-ol |
| 2-amine | |
| 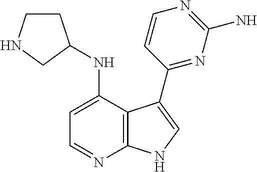 | 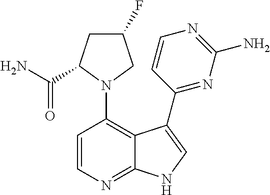 |
| 3-(2-aminopyrimidin-4-yl)-N-pyrrolidin-3- | (4S)-1-[3-(2-aminopyrimidin-4-yl)-1H- |
| yl-1H-pyrrolo[2,3-b]pyridin-4-amine | pyrrolo[2,3-b]pyridin-4-yl]-4-fluoro-L- |
| | prolinamide |
| 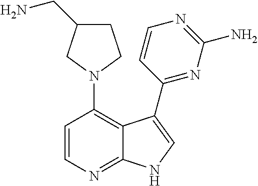 | 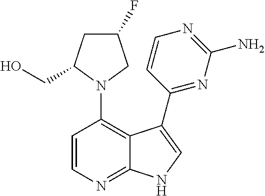 |
| 4-{4-[3-(aminomethyl)pyrrolidin-1-yl]- | {(2S,4S)-1-[3-(2-aminopyrimidin-4-yl)- |
| 1H-pyrrolo[2,3-b]pyridin-3-yl}pyrimidin- | 1H-pyrrolo[2,3-b]pyridin-4-yl]-4- |
| 2-amine | fluoropyrrolidin-2-yl}methanol |
| 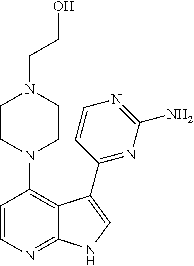 | 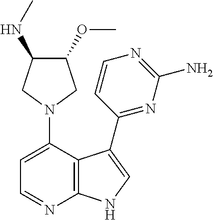 |
| 2-{4-[3-(2-aminopyrimidin-4-yl)-1H- | 4-{4-[(3R,4R)-3-(methylamino)-4- |
| pyrrolo[2,3-b]pyridine-4-yl]piperazin-1- | (methyloxy)pyrrolidin-1-yl]-1H- |
| yl}ethanol | pyrrolo[2,3-b]pyridin-3-yl}pyrimidin-2- |
| | amine |
| 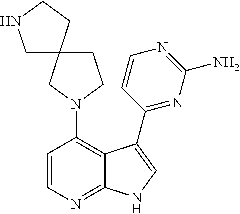 | 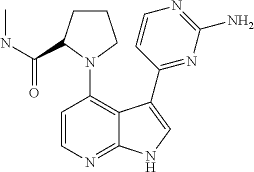 |
| 4-[4-(2,7-diazaspiro[4.4]non-2-yl)-1H- | 1-[3-(2-aminopyrimidin-4-yl)-1H- |
| pyrrolo[2,3-b]pyridin-3-yl]pyrimidin-2- | pyrrolo[2,3-b]pyridin-4-yl]-N-methyl-L- |
| amine | prolinamide |
| 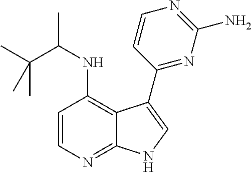 | 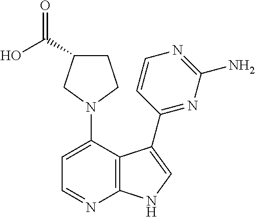 |
| 3-(2-aminopyrimidin-4-yl)-N-(1,2,2- | (3R)-1-[3-(2-aminopyrimidin-4-yl)-1H- |
| trimethylpropyl)-1H-pyrrolo[2,3- | pyrrolo[2,3-b]pyridin-4-yl]pyrrolidine-3- |
| b]pyridin-4-amine | carboxylic acid |
| 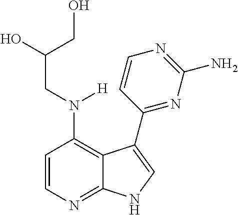 | 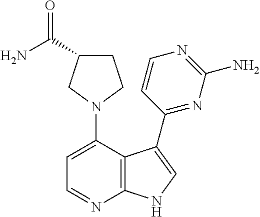 |
| 3-{[3-(2-aminopyrimidin-4-yl)-1H- | (3R)-1-[3-(2-aminopyrimidin-4-yl)-1H- |
| pyrrolo[2,3-b]pyridine-4- | pyrrolo[2,3-b]pyridin-4-yl]pyrrolidine-3- |
| yl]amino}propane-1,2-diol | carboxamide |
| 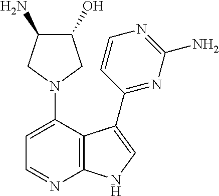 | 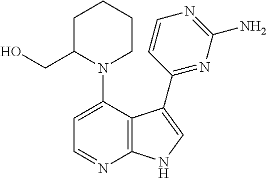 |
| (3R,4R)-4-amino-1-[3-(2- | {1-[3-(2-aminopyrimidin-4-yl)-1H- |
| aminopyrimidin-4-yl)-1H-pyrrolo[2,3- | pyrrolo[2,3-b]pyridin-4-yl]piperidin-2- |
| b]pyridin-4-yl]pyrrolidin-3-ol | yl}methanol |
| 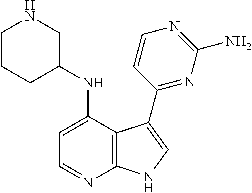 | 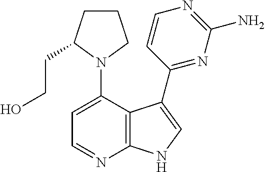 |
| 3-(2-aminopyrimidin-4-yl)-N-piperidin-3- | 2-{(2S)-1-[3-(2-aminopyrimidin-4-yl)- |
| yl-1H-pyrrolo[2,3-b]pyridin-4-amine | 1H-pyrrolo[2,3-b]pyridin-4- |
| | yl]pyrrolidin-2-yl}ethanol |
| 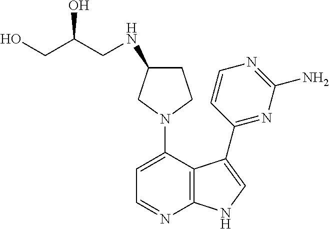 | 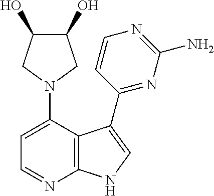 |
| (2S)-3-({(3S)-1-[3-(2-aminopyrimidin-4- | (3R,4S)-1-[3-(2-aminopyrimidin-4-yl)- |
| yl)-1H-pyrrolo[2,3-b]pyridin-4- | 1H-pyrrolo[2,3-b]pyridin-4- |
| yl]pyrrolidin-3-yl}amino)propane-1,2-diol | yl]pyrrolidine-3,4-diol |
| 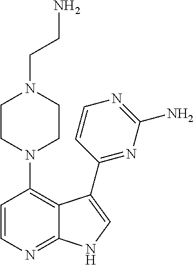 | 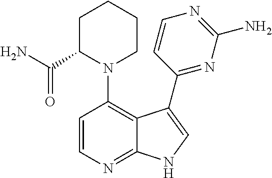 |
| 4-{4-[4-(2-aminoethyl)piperazin-1-yl]- | (2S)-1-[3-(2-aminopyrimidin-4-yl)-1H- |
| 1H-pyrrolo[2,3-b]pyridin-3-yl}pyrimidin- | pyrrolo[2,3-b]pyridin-4-yl]piperidine-2- |
| 2-amine | carboxamide |
| 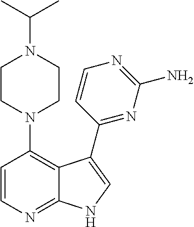 | 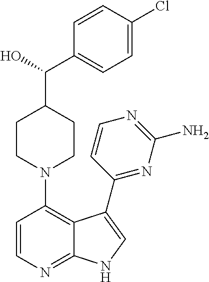 |
| 4-{4-[4-(1-methylethyl)piperazin-1-yl]- | (S)-{1-[3-(2-aminopyrimidin-4-yl)-1H- |
| 1H-pyrrolo[2,3-b]pyridin-3-yl}pyrimidin- | pyrrolo[2,3-b]pyridin-4-yl]piperidin-4- |
| 2-amine | yl}(4-chlorophenyl)methanol |
| 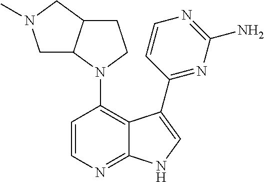 | 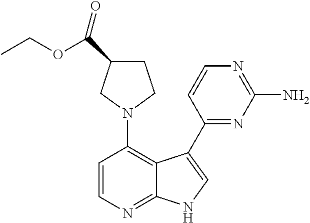 |
| 4-[4-(5-methylhexahydropyrrolo[3,4- | ethyl (3S)-1-[3-(2-aminopyrimidin-4- |
| b]pyrrol-1(2H)-yl)-1H-pyrrolo[2,3- | yl)-1H-pyrrolo[2,3-b]pyridin-4- |
| b]pyridin-3-yl]pyrimidin-2-amine | yl]pyrrolidine-3-carboxylate |
| 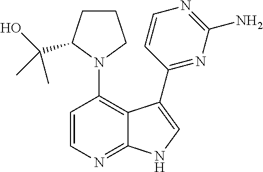 | 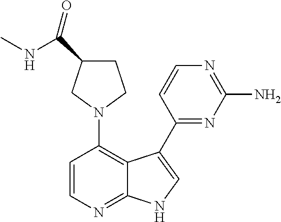 |
| 2-{(2S)-1-[3-(2-aminopyrimidin-4-yl)-1H- | (3S)-1-[3-(2-aminopyrimidin-4-yl)-1H- |
| pyrrolo[2,3-b]pyridin-4-yl]pyrrolidin-2- | pyrrolo[2,3-b]pyridin-4-yl]-N- |
| yl}propan-2-ol | methylpyrrolidine-3-carboxamide |
| 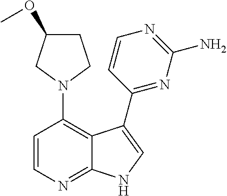 | 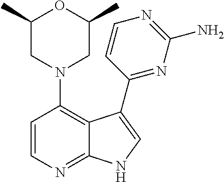 |
| 4-{4-[(3S)-3-(methyloxy)pyrrolidin-1-yl]- | 4-{4-[(2R,6S)-2,6-dimethylmorpholin- |
| 1H-pyrrolo[2,3-b]pyridin-3-yl}pyrimidin- | 4-yl]-1H-pyrrolo[2,3-b]pyridin-3- |
| 2-amine | yl}pyrimidin-2-amine |
| 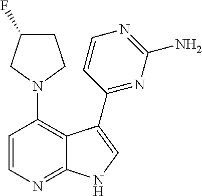 | 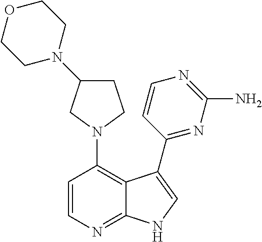 |
| 4-{4-[(3R)-3-fluoropyrrolidin-1-yl]-1H- | 4-[4-(3-morpholin-4-ylpyrrolidin-1-yl)- |
| pyrrolo[2,3-b]pyridin-3-yl}pyrimidin-2- | 1H-pyrrolo[2,3-b]pyridin-3- |
| amine | yl]pyrimidin-2-amine |
| 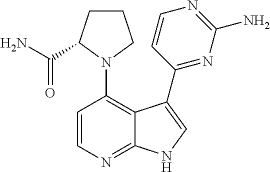 | 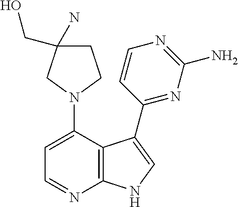 |
| 1-[3-(2-aminopyrimidin-4-yl)-1H- | {3-amino-1-[3-(2-aminopyrimidin-4-yl)- |
| pyrrolo[2,3-b]pyridin-4-yl]-prolinamide | 1H-pyrrolo[2,3-b]pyridin-4- |
| | yl]pyrrolidin-3-yl}methanol |
| 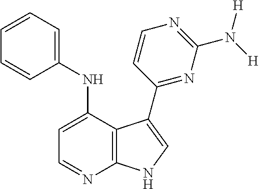 | 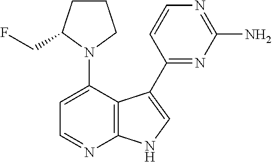 |
| 3-(2-aminopyrimidin-4-yl)-N-phenyl-1H- | 4-{4-[(2S)-2-(fluoromethyl)pyrrolidin-1- |
| pyrrolo[2,3-b]pyridin-4-amine | yl]-1H-pyrrolo[2,3-b]pyridin-3- |
| | yl}pyrimidin-2-amine |
| 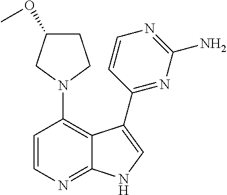 | 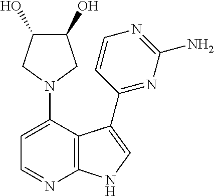 |
| 4-{4-[(3R)-3-(methyloxy)pyrrolidin-1-yl]- | (3S,4S)-1-[3-(2-aminopyrimidin-4-yl)- |
| 1H-pyrrolo[2,3-b]pyridin-3-yl}pyrimidin- | 1H-pyrrolo[2,3-b]pyridin-4- |
| 2-amine | yl]pyrrolidine-3,4-diol |
| 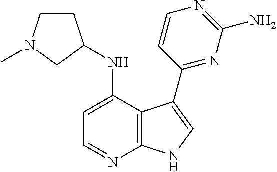 | 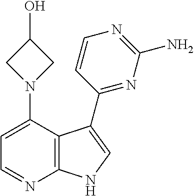 |
| 3-(2-aminopyrimidin-4-yl)-N-(1- | 1-[3-(2-aminopyrimidin-4-yl)-1H- |
| methylpyrrolidin-3-yl)-1H-pyrrolo[2,3- | pyrrolo[2,3-b]pyridin-4-yl]azetidin-3-ol |
| b]pyridin-4-amine | |
| 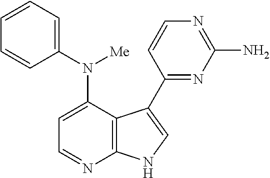 | 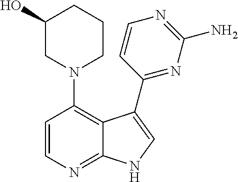 |
| 3-(2-aminopyrimidin-4-yl)-N-methyl-N- | (3S)-1-[3-(2-aminopyrimidin-4-yl)-1H- |
| phenyl-1H-pyrrolo[2,3-b]pyridin-4-amine | pyrrolo[2,3-b]pyridin-4-yl]piperidin-3-ol |
| 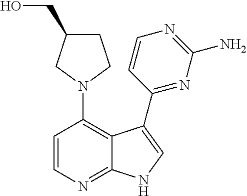 | 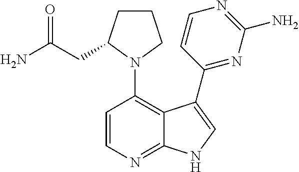 |
| {(3S)-1-[3-(2-aminopyrimidin-4-yl)-1H- | 2-{(2S)-1-[3-(2-aminopyrimidin-4-yl)- |
| pyrrolo[2,3-b]pyridin-4-yl]pyrrolidin-3- | 1H-pyrrolo[2,3-b]pyridin-4- |
| yl}methanol | yl]pyrrolidin-2-yl}acetamide |
| 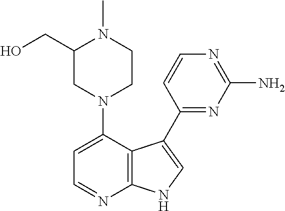 | 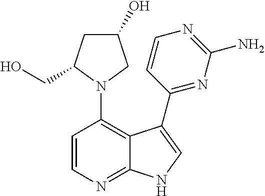 |
| {4-[3-(2-aminopyrimidin-4-yl)-1H- | (3S,5S)-1-[3-(2-aminopyrimidin-4-yl)- |
| pyrrolo[2,3-b]pyridin-4-yl]-1- | 1H-pyrrolo[2,3-b]pyridin-4-yl]-5- |
| methylpiperazin-2-yl}methanol | (hydroxymethyl)pyrrolidin-3-ol |
| 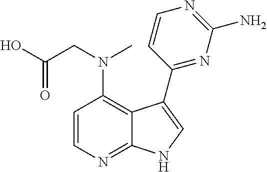 | 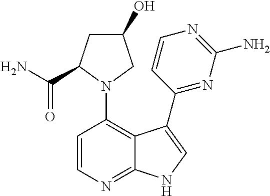 |
| N-[3-(2-aminopyrimidin-4-yl)-1H- | (4S)-1-[3-(2-aminopyrimidin-4-yl)-1H- |
| pyrrolo[2,3-b]pyridin-4-yl]-N- | pyrrolo[2,3-b]pyridin-4-yl]-4-hydroxy- |
| methylglycine | L-prolinamide |
| 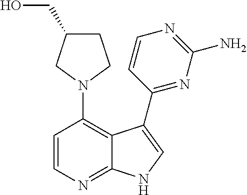 | 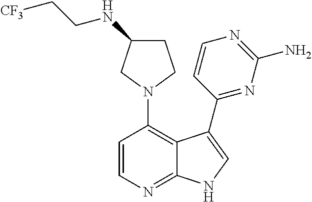 |
| {(3R)-1-[3-(2-aminopyrimidin-4-yl)-1H- | 4-(4-{(3S)-3-[(3,3,3- |
| pyrrolo[2,3-b]pyridin-4-yl]pyrrolidin-3- | trifluoropropyl)amino]pyrrolidin-1-yl}- |
| yl}methanol | 1H-pyrrolo[2,3-b]pyridin-3- |
| | yl)pyrimidin-2-amine |
| 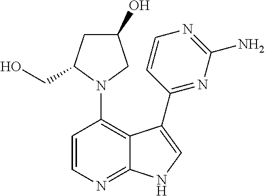 | 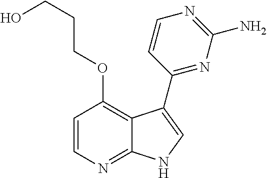 |
| (3R,5S)-1-[3-(2-aminopyrimidin-4-yl)- | 3-{[3-(2-aminopyrimidin-4-yl)-1H-pyrrolo[2,3- |
| 1H-pyrrolo[2,3-b]pyridin-4-yl]-5- | b]pyridin-4-yl]oxy}propan-1-ol |
| (hydroxymethyl)pyrrolidin-3-ol | |
| 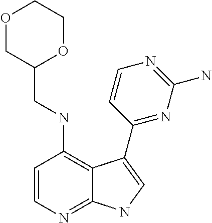 | 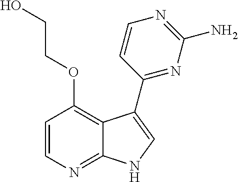 |
| 3-(2-aminopyrimidin-4-yl)-N-(1,4-dioxan-2- | 2-{[3-(2-aminopyrimidin-4-yl)-1H-pyrrolo[2,3- |
| ylmethyl)-1H-pyrrolo[2,3-b]pyridin-4-amine | b]pyridin-4-yl]oxy}ethanol |
| 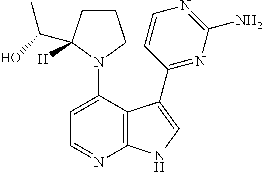 | 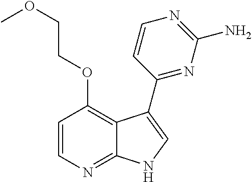 |
| (1R)-1-{(2S)-1-[3-(2-aminopyrimidin-4-yl)- | 4-(4-{[2-(methyloxy)ethyl]oxy}-1H-pyrrolo[2,3- |
| 1H-pyrrolo[2,3-b]pyridin-4-yl]pyrrolidin-2- | b]pyridin-3-yl)pyrimidin-2-amine |
| yl}ethanol | |
| 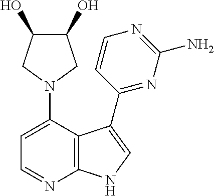 | 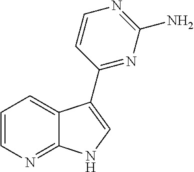 |
| (3R,4R)-1-[3-(2-aminopyrimidin-4-yl)-1H- | 4-(1H-pyrrolo[2,3-b]pyridin-3-yl)pyrimidin-2- |
| pyrrolo[2,3-b]pyridin-4-yl]pyrrolidine-3,4-diol | amine |
| 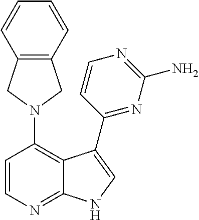 | 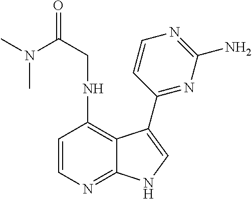 |
| 4-[4-(1,3-dihydro-2H-isoindol-2-yl)-1H- | N²-[3-(2-aminopyrimidin-4-yl)-1H-pyrrolo[2,3- |
| pyrrolo[2,3-b]pyridin-3-yl]pyrimidin-2-amine | b]pyridin-4-yl]-N,N-dimethylglycinamide |
| 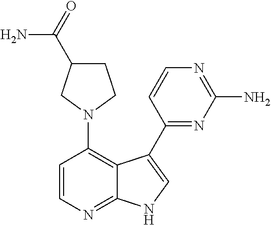 | 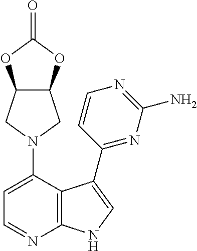 |
| (3S)-1-[3-(2-aminopyrimidin-4-yl)-1H- | (3aR,6aS)-5-[3-(2-aminopyrimidin-4-yl)-1H- |
| pyrrolo[2,3-b]pyridin-4-yl]pyrrolidine-3- | pyrrolo[2,3-b]pyridin-4-yl]tetrahydro-3aH- |
| carboxamide | [1,3]dioxolo[4,5-c]pyrrol-2-one |
| 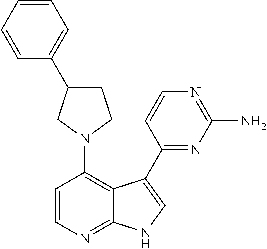 | 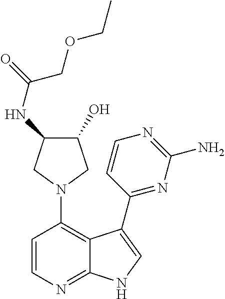 |
| 4-{4-[(3R)-3-phenylpyrrolidin-1-yl]-1H- | N-{(3R,4R)-1-[3-(2-aminopyrimidin-4-yl)-1H- |
| pyrrolo[2,3-b]pyridin-3-yl}pyrimidin-2-amine | pyrrolo[2,3-b]pyridin-4-yl]-4-hydroxypyrrolidin- |
| | 3-yl}-2-(ethyloxy)acetamide |
| 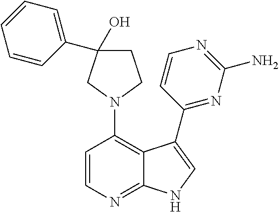 | 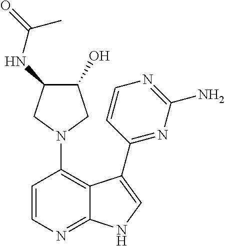 |
| 1-[3-(2-aminopyrimidin-4-yl)-1H-pyrrolo[2,3- | N-{(3R,4R)-1-[3-(2-aminopyrimidin-4-yl)-1H- |
| b]pyridin-4-yl]-3-phenylpyrrolidin-3-ol | pyrrolo[2,3-b]pyridin-4-yl]-4-hydroxypyrrolidin- |
| | 3-yl}acetamide |
| 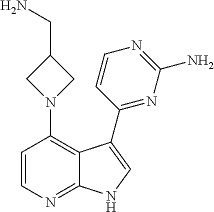 | 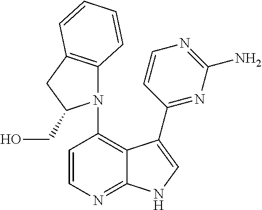 |
| 4-{4-[3-(aminomethyl)azetidin-1-yl]-1H- | {(2S)-1-[3-(2-aminopyrimidin-4-yl)-1H- |
| pyrrolo[2,3-b]pyridin-3-yl}pyrimidin-2-amine | pyrrolo[2,3-b]pyridin-4-yl]-2,3-dihydro-1H- |
| | indol-2-yl}methanol |
| 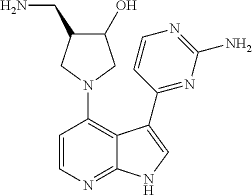 | 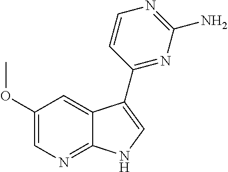 |
| (3R,4S)-4-(aminomethyl)-1-[3-(2- | 4-[5-(methyloxy)-1H-pyrrolo[2,3-b]pyridin-3- |
| aminopyrimidin-4-yl)-1H-pyrrolo[2,3- | yl]pyrimidin-2-amine |
| b]pyridin-4-yl]pyrrolidin-3-ol | |
| 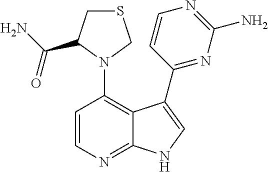 | 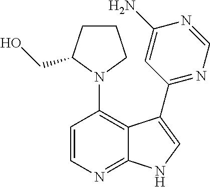 |
| (4R)-3-[3-(2-aminopyrimidin-4-yl)-1H- | {(2S)-1-[3-(6-aminopyrimidin-4-yl)-1H- |
| pyrrolo[2,3-b]pyridin-4-yl]-1,3-thiazolidine-4- | pyrrolo[2,3-b]pyridin-4-yl]pyrrolidin-2- |
| carboxamide | yl}methanol |
| 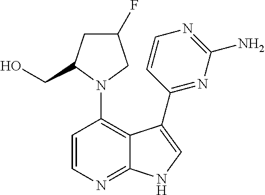 | 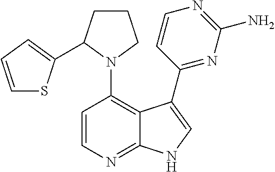 |
| {(2S,4R)-1-[3-(2-aminopyrimidin-4-yl)-1H- | 4-{4-[2-(2-thienyl)pyrrolidin-1-yl]-1H- |
| pyrrolo[2,3-b]pyridin-4-yl]-4-fluoropyrrolidin- | pyrrolo[2,3-b]pyridin-3-yl}pyrimidin-2-amine |
| 2-yl}methanol | |
| 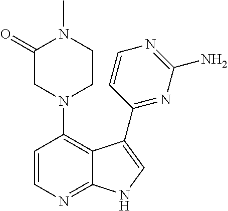 | 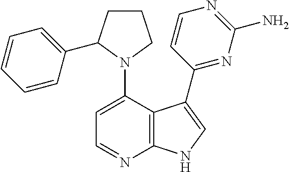 |
| 4-[3-(2-aminopyrimidin-4-yl)-1H-pyrrolo[2,3- | 4-[4-(2-phenylpyrrolidin-1-yl)-1H-pyrrolo[2,3- |
| b]pyridin-4-yl]-1-methylpiperazin-2-one | b]pyridin-3-yl]pyrimidin-2-amine |
| 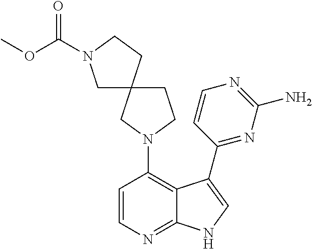 | 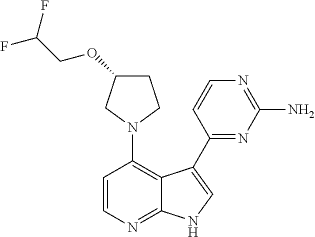 |
| methyl 7-[3-(2-aminopyrimidin-4-yl)-1H- | 4-(4-{(3R)-3-[(2,2-difluoroethyl)oxy]pyrrolidin- |
| pyrrolo[2,3-b]pyridin-4-yl]-2,7- | 1-yl}-1H-pyrrolo[2,3-b]pyridin-3-yl)pyrimidin-2- |
| diazaspiro[4.4]nonane-2-carboxylate | amine |
| 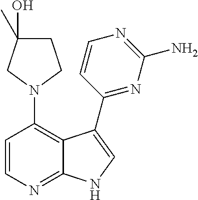 | 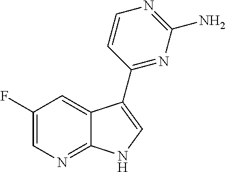 |
| 1-[3-(2-aminopyrimidin-4-yl)-1H-pyrrolo[2,3- | 4-(5-fluoro-1H-pyrrolo[2,3-b]pyridin-3- |
| b]pyridin-4-yl]-3-methylpyrrolidin-3-ol | yl)pyrimidin-2-amine |
| 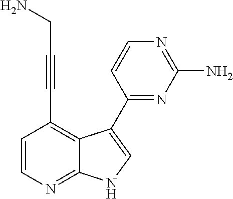 | 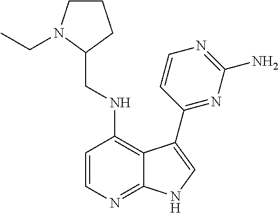 |
| 4-[4-(3-aminoprop-1-yn-1-yl)-1H-pyrrolo[2,3- | 3-(2-aminopyrimidin-4-yl)-N-[(1-ethylpyrrolidin- |
| b]pyridin-3-yl]pyrimidin-2-amine | 2-yl)methyl]-1H-pyrrolo[2,3-b]pyridin-4-amine |
| 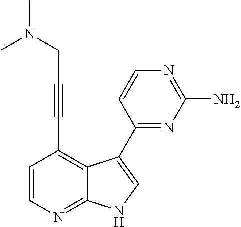 | 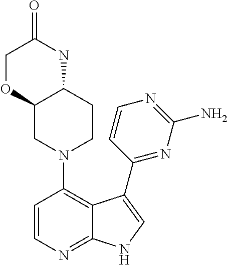 |
| 4-{4-[3-(dimethylamino)prop-1-yn-1-yl]-1H- | (4aR,8aR)-6-[3-(2-aminopyrimidin-4-yl)-1H- |
| pyrrolo[2,3-b]pyridin-3-yl}pyrimidin-2-amine | pyrrolo[2,3-b]pyridin-4-yl]hexahydro-1H- |
| | pyrido[3,4-b][1,4]oxazin-2(3H)-one |
| 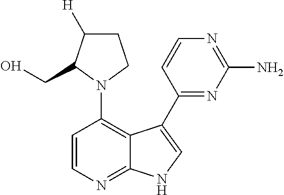 | 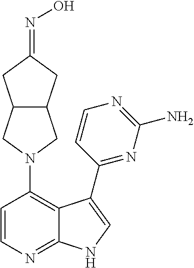 |
| (2R,3S)-1-[3-(2-aminopyrimidin-4-yl)-1H- | 2-[3-(2-aminopyrimidin-4-yl)-1H-pyrrolo[2,3- |
| pyrrolo[2,3-b]pyridin-4-yl]-2- | b]pyridin-4-yl]hexahydrocyclopenta[c]pyrrol- |
| (hydroxymethyl)pyrrolidin-3-ol | 5(1H)-one oxime |
| 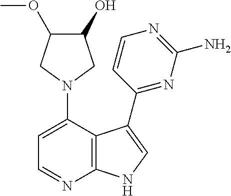 | 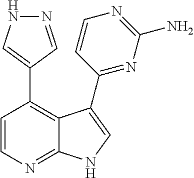 |
| (3R,4R)-1-[3-(2-aminopyrimidin-4-yl)-1H- | 4-[4-(1H-pyrazol-4-yl)-1H-pyrrolo[2,3- |
| pyrrolo[2,3-b]pyridin-4-yl]-4- | b]pyridin-3-yl]pyrimidin-2-amine |
| (methyloxy)pyrrolidin-3-ol | |
| 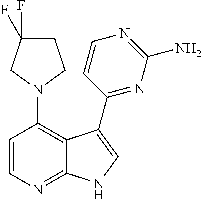 | 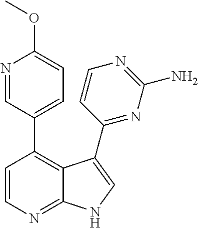 |
| 4-[4-(3,3-difluoropyrrolidin-1-yl)-1H- | 4-{4-[6-(methyloxy)pyridin-3-yl]-1H- |
| pyrrolo[2,3-b]pyridin-3-yl]pyrimidin-2-amine | pyrrolo[2,3-b]pyridin-3-yl}pyrimidin-2-amine |
| 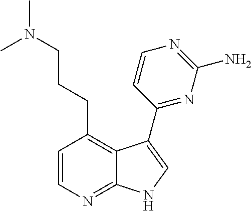 | 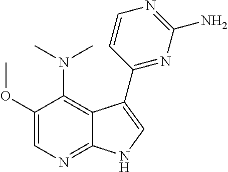 |
| 4-{4-[3-(dimethylamino)propyl]-1H- | 3-(2-aminopyrimidin-4-yl)-N,N-dimethyl-5- |
| pyrrolo[2,3-b]pyridin-3-yl}pyrimidin-2-amine | (methyloxy)-1H-pyrrolo[2,3-b]pyridin-4-amine |
| 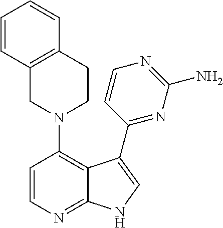 | 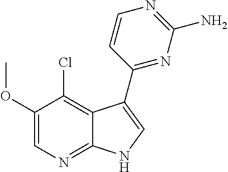 |
| 4-[4-(3,4-dihydroisoquinolin-2(1H)-yl)-1H- | 4-[4-chloro-5-(methyloxy)-1H-pyrrolo[2,3- |
| pyrrolo[2,3-b]pyridin-3-yl]pyrimidin-2-amine | b]pyridin-3-yl]pyrimidin-2-amine |
| 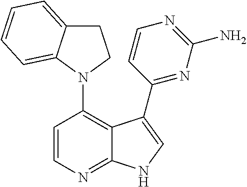 | 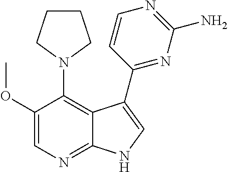 |
| 4-[4-(2,3-dihydro-1H-indol-1-yl)-1H- | 4-[5-(methyloxy)-4-pyrrolidin-1-yl-1H- |
| pyrrolo[2,3-b]pyridin-3-yl]pyrimidin-2-amine | pyrrolo[2,3-b]pyridin-3-yl]pyrimidin-2-amine |
| 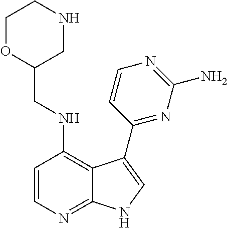 | 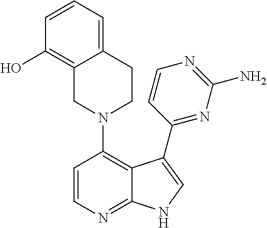 |
| 3-(2-aminopyrimidin-4-yl)-N-(morpholin-2- | 2-[3-(2-aminopyrimidin-4-yl)-1H-pyrrolo[2,3- |
| ylmethyl)-1H-pyrrolo[2,3-b]pyridin-4-amine | b]pyridin-4-yl]-1,2,3,4-tetrahydroisoquinolin-8- |
| | ol |
| 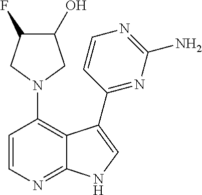 | 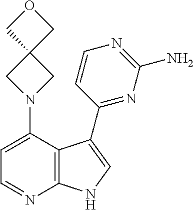 |
| (3S,4S)-1-[3-(2-aminopyrimidin-4-yl)-1H- | 4-[4-(2-oxa-6-azaspiro[3.3]hept-6-yl)-1H- |
| pyrrolo[2,3-b]pyridin-4-yl]-4-fluoropyrrolidin- | pyrrolo[2,3-b]pyridin-3-yl]pyrimidin-2-amine |
| 3-ol | |
| 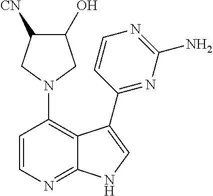 | 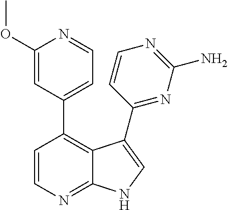 |
| (3R,4R)-1-[3-(2-aminopyrimidin-4-yl)-1H- | 4-{4-[2-(methyloxy)pyridin-4-yl]-1H- |
| pyrrolo[2,3-b]pyridin-4-yl]-4- | pyrrolo[2,3-b]pyridin-3-yl}pyrimidin-2-amine |
| hydroxypyrrolidine-3-carbonitrile | |
| 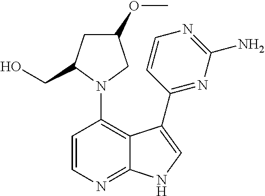 | 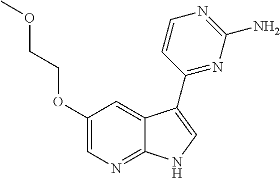 |
| [(2S,4S)-1-[3-(2-aminopyrimidin-4-yl)-1H- | 4-(5-{[2-(methyloxy)ethyl]oxy}-1H-pyrrolo[2,3- |
| pyrrolo[2,3-b]pyridin-4-yl]-4- | b]pyridin-3-yl)pyrimidin-2-amine |
| (methyloxy)pyrrolidin-2-yl]methanol | |
| 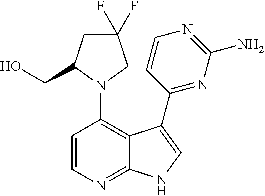 | 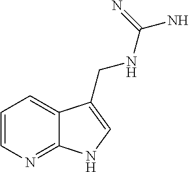 |
| {(2S)-1-[3-(2-aminopyrimidin-4-yl)-1H- | 1-(1H-pyrrolo[2,3-b]pyridin-3- |
| pyrrolo[2,3-b]pyridin-4-yl]-4,4- | ylmethyl)guanidine |
| difluoropyrrolidin-2-yl}methanol | |
| 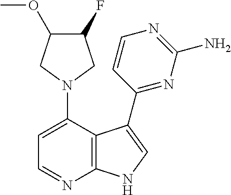 | 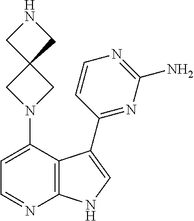 |
| 4-{4-[(3R,4R)-3-fluoro-4- | 4-[4-(2,6-diazaspiro[3.3]hept-2-yl)-1H- |
| (methyloxy)pyrrolidin-1-yl]-1H-pyrrolo[2,3- | pyrrolo[2,3-b]pyridin-3-yl]pyrimidin-2-amine |
| b]pyridin-3-yl}pyrimidin-2-amine | |
| 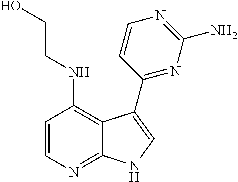 | 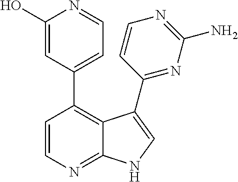 |
| 2-{[3-(2-aminopyrimidin-4-yl)-1H-pyrrolo[2,3- | 4-[3-(2-aminopyrimidin-4-yl)-1H-pyrrolo[2,3- |
| b]pyridin-4-yl]amino}ethanol | b]pyridin-4-yl]pyridin-2-ol |
| 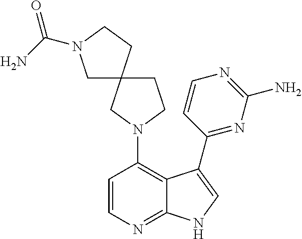 | 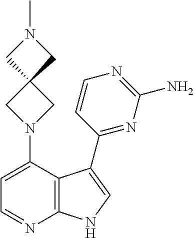 |
| 7-[3-(2-aminopyrimidin-4-yl)-1H-pyrrolo[2,3- | 4-[4-(6-methyl-2,6-diazaspiro[3.3]hept-2-yl)- |
| b]pyridin-4-yl]-2,7-diazaspiro[4.4]nonane-2- | 1H-pyrrolo[2,3-b]pyridin-3-yl]pyrimidin-2- |
| carboxamide | amine |
| 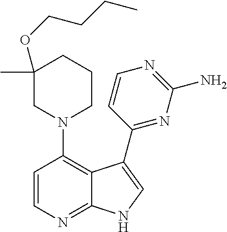 | 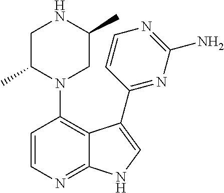 |
| 4-{4-[3-(butyloxy)-3-methylpiperidin-1-yl]- | 4-{4-[(2R,5S)-2,5-dimethylpiperazin-1-yl]-1H- |
| 1H-pyrrolo[2,3-b]pyridin-3-yl}pyrimidin-2- | pyrrolo[2,3-b]pyridin-3-yl}pyrimidin-2-amine |
| amine | |
| 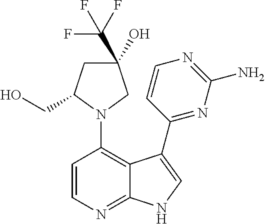 | 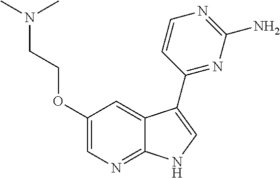 |
| (3S,5S)-1-[3-(2-aminopyrimidin-4-yl)-1H- | 4-(5-{[2-(dimethylamino)ethyl]oxy}-1H- |
| pyrrolo[2,3-b]pyridin-4-yl]-5- | pyrrolo[2,3-b]pyridin-3-yl)pyrimidin-2-amine |
| (hydroxymethyl)-3- | |
| (trifluoromethyl)pyrrolidin-3-ol | |
| 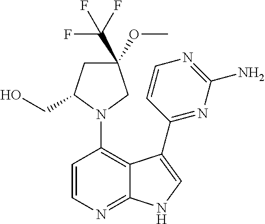 | 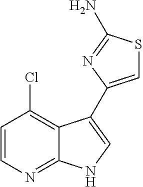 |
| [(2S,4S)-1-[3-(2-aminopyrimidin-4-yl)-1H- | 4-(4-chloro-1H-pyrrolo[2,3-b]pyridin-3-yl)-1,3- |
| pyrrolo[2,3-b]pyridin-4-yl]-4-(methyloxy)-4- | thiazol-2-amine |
| (trifluoromethyl)pyrrolidin-2-yl]methanol | |
| 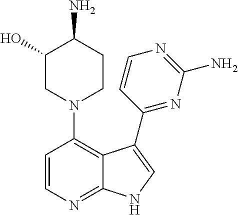 | 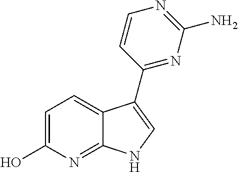 |
| (3S,4S)-4-amino-1-[3-(2-aminopyrimidin-4- | 3-(2-aminopyrimidin-4-yl)-1H-pyrrolo[2,3- |
| yl)-1H-pyrrolo[2,3-b]pyridin-4-yl]piperidin-3- | b]pyridin-6-ol |
| ol | |
| 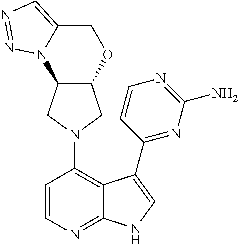 | 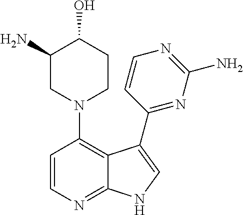 |
| 4-{4-[(5aR,8aR)-5a,6,8,8a-tetrahydro- | (3R,4R)-3-amino-1-[3-(2-aminopyrimidin-4-yl)- |
| 4H,7H-pyrrolo[3,4-b][1,2,3]triazolo[1,5- | 1H-pyrrolo[2,3-b]pyridin-4-yl]piperidin-4-ol |
| d][1,4]oxazin-7-yl]-1H-pyrrolo[2,3-b]pyridin- | |
| 3-yl}pyrimidin-2-amine | |
| 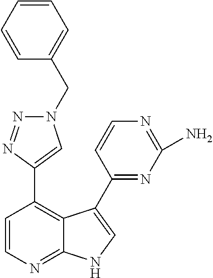 | 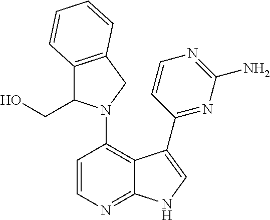 |
| 4-{4-[1-(phenylmethyl)-1H-1,2,3-triazol-4-yl]- | {2-[3-(2-aminopyrimidin-4-yl)-1H-pyrrolo[2,3- |
| 1H-pyrrolo[2,3-b]pyridin-3-yl}pyrimidin-2- | b]pyridin-4-yl]-2,3-dihydro-1H-isoindol-1- |
| amine | yl}methanol |
| 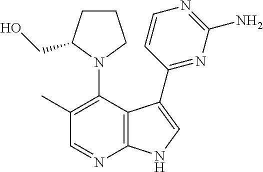 | 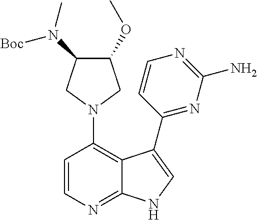 |
| {(2S)-1-[3-(2-aminopyrimidin-4-yl)-5-methyl- | 1,1-dimethylethyl [(3R,4R)-1-[3-(2- |
| 1H-pyrrolo[2,3-b]pyridin-4-yl]pyrrolidin-2- | aminopyrimidin-4-yl)-1H-pyrrolo[2,3-b]pyridin- |
| yl}methanol | 4-yl]-4-(methyloxy)pyrrolidin-3- |
| | yl]methylcarbamate |
| 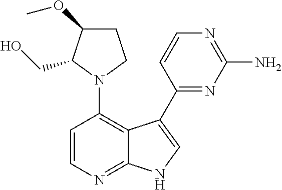 | 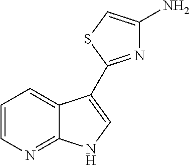 |
| [(2R,3S)-1-[3-(2-aminopyrimidin-4-yl)-1H- | 2-(1H-pyrrolo[2,3-b]pyridin-3-yl)-1,3-thiazol-4- |
| pyrrolo[2,3-b]pyridin-4-yl]-3- | amine |
| (methyloxy)pyrrolidin-2-yl]methanol | |
| 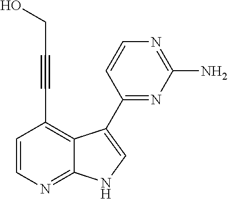 | 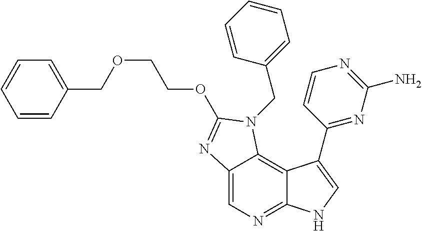 |
| 3-[3-(2-aminopyrimidin-4-yl)-1H-pyrrolo[2,3- | 4-[1-(phenylmethyl)-2-({2- |
| b]pyridin-4-yl]prop-2-yn-1-ol | [(phenylmethyl)oxy]ethyl}oxy)-1,6- |
| | dihydroimidazo[4,5-d]pyrrolo[2,3-b]pyridin-8- |
| | yl]pyrimidin-2-amine |
| 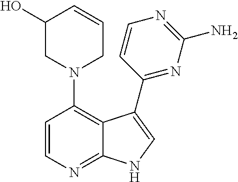 | 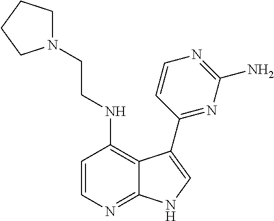 |
| 1-[3-(2-aminopyrimidin-4-yl)-1H-pyrrolo[2,3- | 3-(2-aminopyrimidin-4-yl)-N-(2-pyrrolidin-1- |
| b]pyridin-4-yl]-1,2,3,6-tetrahydropyridin-3-ol | ylethyl)-1H-pyrrolo[2,3-b]pyridin-4-amine |
| 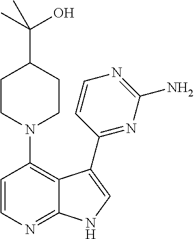 | 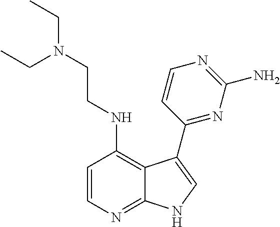 |
| 2-{1-[3-(2-aminopyrimidin-4-yl)-1H- | N′-[3-(2-aminopyrimidin-4-yl)-1H-pyrrolo[2,3- |
| pyrrolo[2,3-b]pyridin-4-yl]piperidin-4- | b]pyridin-4-yl]-N,N-diethylethane-1,2-diamine |
| yl}propan-2-ol | |
| 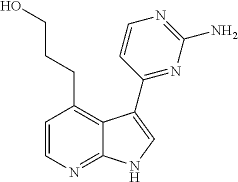 | 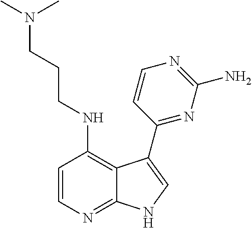 |
| 3-[3-(2-aminopyrimidin-4-yl)-1H-pyrrolo[2,3- | N′-[3-(2-aminopyrimidin-4-yl)-1H-pyrrolo[2,3- |
| b]pyridin-4-yl]propan-1-ol | b]pyridin-4-yl]-N,N-dimethylpropane-1,3- |
| | diamine |
| 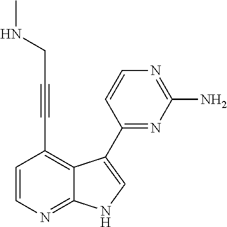 | 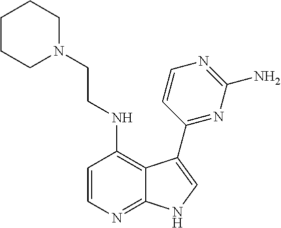 |
| 4-{4-[3-(methylamino)prop-1-yn-1-yl]-1H- | 3-(2-aminopyrimidin-4-yl)-N-(2-piperidin-1- |
| pyrrolo[2,3-b]pyridin-3-yl}pyrimidin-2-amine | ylethyl)-1H-pyrrolo[2,3-b]pyridin-4-amine |
| 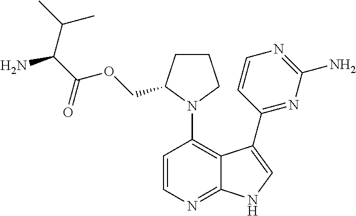 | 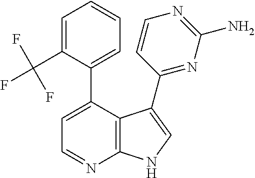 |
| {(2S)-1-[3-(2-aminopyrimidin-4-yl)-1H- | 4-{4-[2-(trifluoromethyl)phenyl]-1H-pyrrolo[2,3- |
| pyrrolo[2,3-b]pyridin-4-yl]pyrrolidin-2- | b]pyridin-3-yl}pyrimidin-2-amine |
| yl}methyl L-valinate | |
| 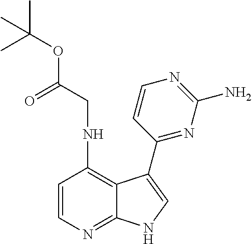 | 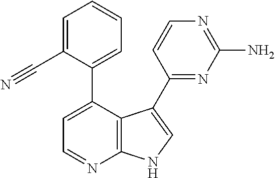 |
| 1,1-dimethylethyl N-[3-(2-aminopyrimidin-4- | 2-[3-(2-aminopyrimidin-4-yl)-1H-pyrrolo[2,3- |
| yl)-1H-pyrrolo[2,3-b]pyridin-4-yl]glycinate | b]pyridin-4-yl]benzonitrile |
| 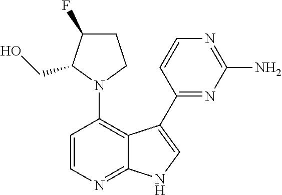 | 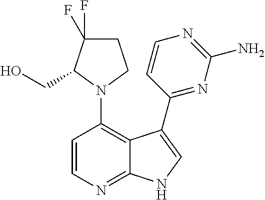 |
| {(2R,3S)-1-[3-(2-aminopyrimidin-4-yl)-1H- | {(2R)-1-[3-(2-aminopyrimidin-4-yl)-1H- |
| pyrrolo[2,3-b]pyridin-4-yl]-3-fluoropyrrolidin- | pyrrolo[2,3-b]pyridin-4-yl]-3,3- |
| 2-yl}methanol | difluoropyrrolidin-2-yl}methanol |
| 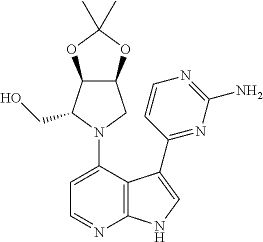 | 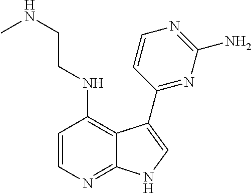 |
| {(3aR,4R,6aS)-5-[3-(2-aminopyrimidin-4-yl)- | N-[3-(2-aminopyrimidin-4-yl)-1H-pyrrolo[2,3- |
| 1H-pyrrolo[2,3-b]pyridin-4-yl]-2,2- | b]pyridin-4-yl]-N′-methylethane-1,2-diamine |
| dimethyltetrahydro-3aH-[1,3]dioxolo[4,5- | |
| c]pyrrol-4-yl}methanol | |
| 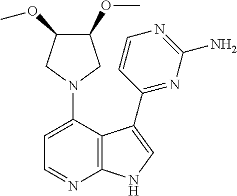 | 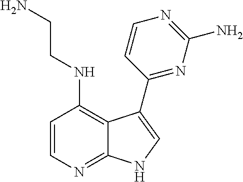 |
| 4-{4-[(3R,4S)-3,4-bis(methyloxy)pyrrolidin-1- | N-[3-(2-aminopyrimidin-4-yl)-1H-pyrrolo[2,3- |
| yl]-1H-pyrrolo[2,3-b]pyridin-3-yl}pyrimidin-2- | b]pyridin-4-yl]ethane-1,2-diamine |
| amine | |
| 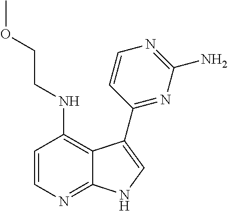 | 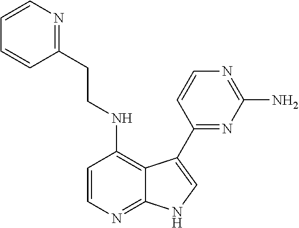 |
| 3-(2-aminopyrimidin-4-yl)-N-[2- | 3-(2-aminopyrimidin-4-yl)-N-(2-pyridin-2- |
| (methyloxy)ethyl]-1H-pyrrolo[2,3-b]pyridin-4- | ylethyl)-1H-pyrrolo[2,3-b]pyridin-4-amine |
| amine | |
| 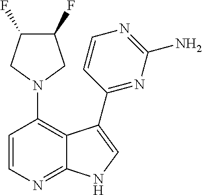 | 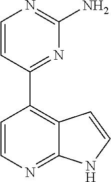 |
| 4-{4-[(3S,4S)-3,4-difluoropyrrolidin-1-yl]-1H- | 4-(1H-pyrrolo[2,3-b]pyridin-4-yl)pyrimidin-2- |
| pyrrolo[2,3-b]pyridin-3-yl}pyrimidin-2-amine | amine |
| 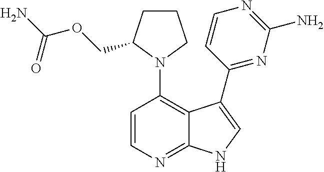 | 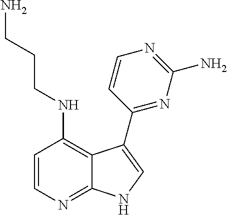 |
| {(2S)-1-[3-(2-aminopyrimidin-4-yl)-1H- | N-[3-(2-aminopyrimidin-4-yl)-1H-pyrrolo[2,3- |
| pyrrolo[2,3-b]pyridin-4-yl]pyrrolidin-2- | b]pyridin-4-yl]propane-1,3-diamine |
| yl}methyl carbamate | |
| 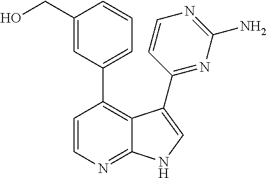 | 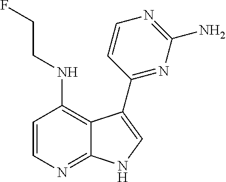 |
| {3-[3-(2-aminopyrimidin-4-yl)-1H-pyrrolo[2,3- | 3-(2-aminopyrimidin-4-yl)-N-(2-fluoroethyl)- |
| b]pyridin-4-yl]phenyl}methanol | 1H-pyrrolo[2,3-b]pyridin-4-amine |
| 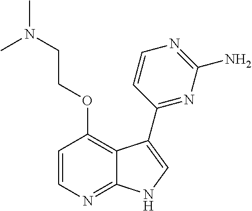 | 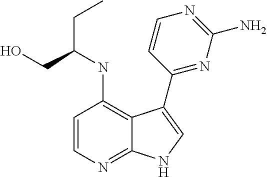 |
| 4-(4-{[2-(dimethylamino)ethyl]oxy}-1H- | (2R)-2-{[3-(2-aminopyrimidin-4-yl)-1H- |
| pyrrolo[2,3-b]pyridin-3-yl)pyrimidin-2-amine | pyrrolo[2,3-b]pyridin-4-yl]amino}butan-1-ol |
| 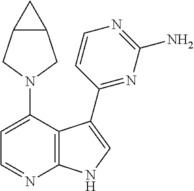 | 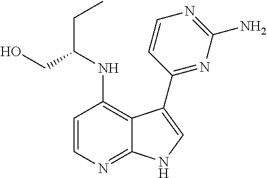 |
| 4-[4-(3-azabicyclo[3.1.0]hex-3-yl)-1H- | (2S)-2-{[3-(2-aminopyrimidin-4-yl)-1H- |
| pyrrolo[2,3-b]pyridin-3-yl]pyrimidin-2-amine | pyrrolo[2,3-b]pyridin-4-yl]amino}butan-1-ol |
| 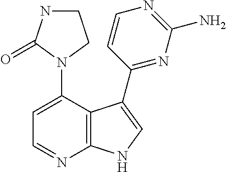 | 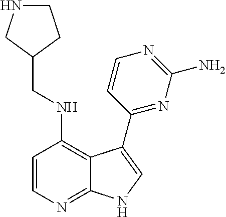 |
| 1-[3-(2-aminopyrimidin-4-yl)-1H-pyrrolo[2,3- | 3-(2-aminopyrimidin-4-yl)-N-(pyrrolidin-3- |
| b]pyridin-4-yl]imidazolidin-2-one | ylmethyl)-1H-pyrrolo[2,3-b]pyridin-4-amine |
| 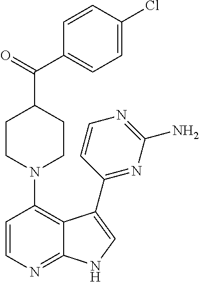 | 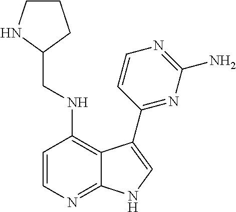 |
| {1-[3-(2-aminopyrimidin-4-yl)-1H-pyrrolo[2,3- | 3-(2-aminopyrimidin-4-yl)-N-(pyrrolidin-2- |
| b]pyridin-4-yl]piperidin-4-yl}(4- | ylmethyl)-1H-pyrrolo[2,3-b]pyridin-4-amine |
| chlorophenyl)methanone | |
| 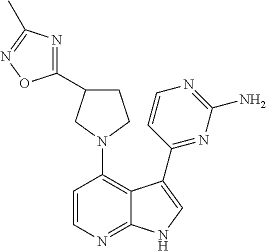 | 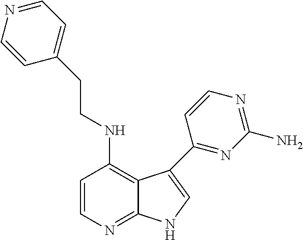 |
| 4-{4-[3-(3-methyl-1,2,4-oxadiazol-5- | 3-(2-aminopyrimidin-4-yl)-N-(2-pyridin-4- |
| yl)pyrrolidin-1-yl]-1H-pyrrolo[2,3-b]pyridin-3- | ylethyl)-1H-pyrrolo[2,3-b]pyridin-4-amine |
| yl}pyrimidin-2-amine | |
| 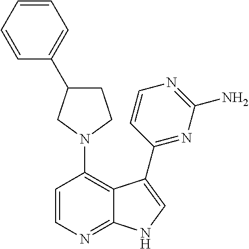 | 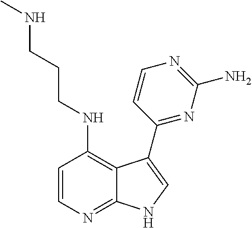 |
| 4-[4-(3-phenylpyrrolidin-1-yl)-1H-pyrrolo[2,3- | N-[3-(2-aminopyrimidin-4-yl)-1H-pyrrolo[2,3- |
| b]pyridin-3-yl]pyrimidin-2-amine | b]pyridin-4-yl]-N′-methylpropane-1,3-diamine |
| 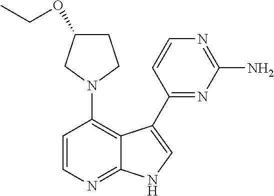 | 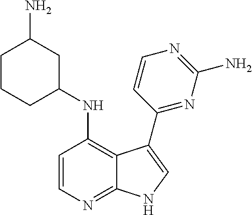 |
| 4-{4-[(3R)-3-(ethyloxy)pyrrolidin-1-yl]-1H- | N-[3-(2-aminopyrimidin-4-yl)-1H-pyrrolo[2,3- |
| pyrrolo[2,3-b]pyridin-3-yl}pyrimidin-2-amine | b]pyridin-4-yl]cyclohexane-1,3-diamine |
| 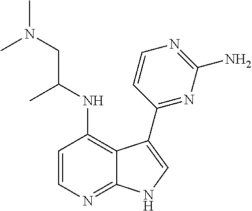 | 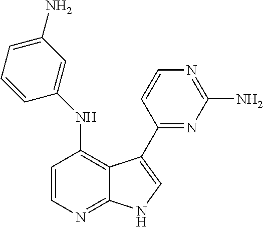 |
| N²-[3-(2-aminopyrimidin-4-yl)-1H- | N-[3-(2-aminopyrimidin-4-yl)-1H-pyrrolo[2,3- |
| pyrrolo[2,3-b]pyridin-4-yl]-N¹,N¹- | b]pyridin-4-yl]benzene-1,3-diamine |
| dimethylpropane-1,2-diamine | |
| 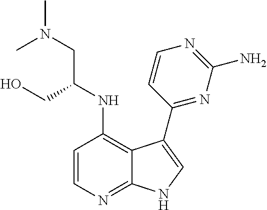 | 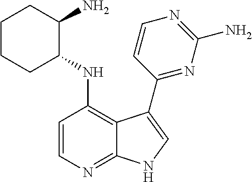 |
| (2S)-2-{[3-(2-aminopyrimidin-4-yl)-1H- | (1R,2R)-N-[3-(2-aminopyrimidin-4-yl)-1H- |
| pyrrolo[2,3-b]pyridin-4-yl]amino}-3- | pyrrolo[2,3-b]pyridin-4-yl]cyclohexane-1,2- |
| (dimethylamino)propan-1-ol | diamine |
| 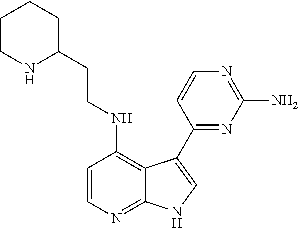 | 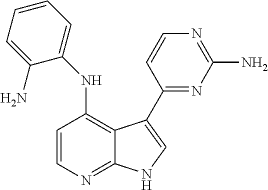 |
| 3-(2-aminopyrimidin-4-yl)-N-(2-piperidin-2- | N-[3-(2-aminopyrimidin-4-yl)-1H-pyrrolo[2,3- |
| ylethyl)-1H-pyrrolo[2,3-b]pyridin-4-amine | b]pyridin-4-yl]benzene-1,2-diamine |
| 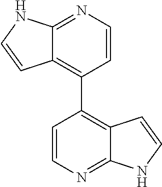 | 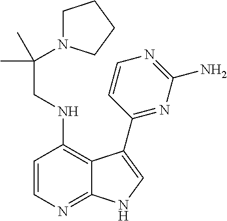 |
| 1H,1′H-4,4′-bipyrrolo[2,3-b]pyridine | 3-(2-aminopyrimidin-4-yl)-N-(2-methyl-2- |
| | pyrrolidin-1-ylpropyl)-1H-pyrrolo[2,3-b]pyridin- |
| | 4-amine |
| 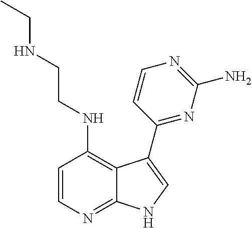 | 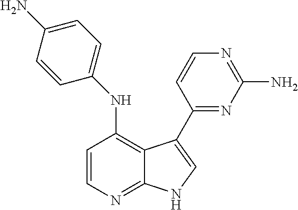 |
| N-[3-(2-aminopyrimidin-4-yl)-1H-pyrrolo[2,3- | N-[3-(2-aminopyrimidin-4-yl)-1H-pyrrolo[2,3- |
| b]pyridin-4-yl]-N′-ethylethane-1,2-diamine | b]pyridin-4-yl]benzene-1,4-diamine |
| 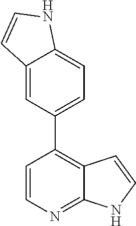 | 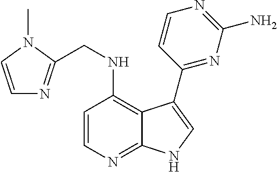 |
| 4-(1H-indol-5-yl)-1H-pyrrolo[2,3-b]pyridine | 3-(2-aminopyrimidin-4-yl)-N-[(1-methyl-1H- |
| | imidazol-2-yl)methyl]-1H-pyrrolo[2,3-b]pyridin- |
| | 4-amine |
| 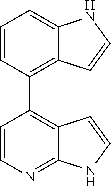 | 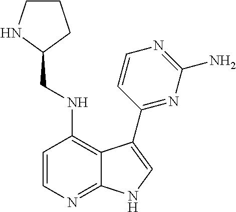 |
| 4-(1H-indol-4-yl)-1H-pyrrolo[2,3-b]pyridine | 3-(2-aminopyrimidin-4-yl)-N-[(2S)-pyrrolidin-2- |
| | ylmethyl]-1H-pyrrolo[2,3-b]pyridin-4-amine |
| 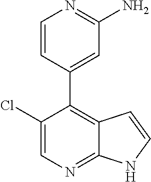 | 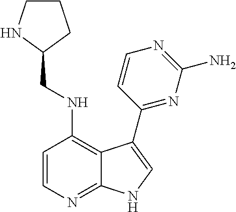 |
| 4-(5-chloro-1H-pyrrolo[2,3-b]pyridin-4- | 3-(2-aminopyrimidin-4-yl)-N-[(2R)-pyrrolidin-2- |
| yl)pyridin-2-amine | ylmethyl]-1H-pyrrolo[2,3-b]pyridin-4-amine |
| | 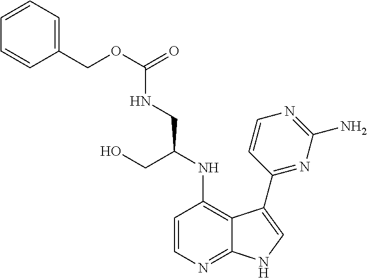 |
| | phenylmethyl [(2R)-2-{[3-(2-aminopyrimidin-4- |
| | yl)-1H-pyrrolo[2,3-b]pyridin-4-yl]amino}-3- |
| | hydroxypropyl]carbamate |

TABLE 1B

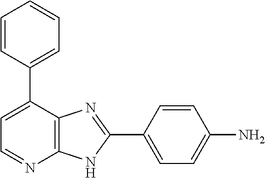

4-(7-phenyl-3H-imidazo[4,5-b]pyridin-2-yl)aniline

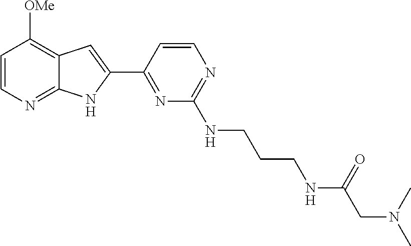

N²,N²-dimethyl-N-[3-({4-[4-(methyloxy)-1H-
pyrrolo[2,3-b]pyridin-2-yl]pyrimidin-2-yl}amino)propyl]glycinamide

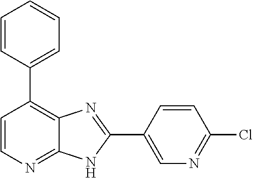

2-(6-chloropyridin-3-yl)-7-phenyl-3H-
imidazo[4,5-b]pyridine

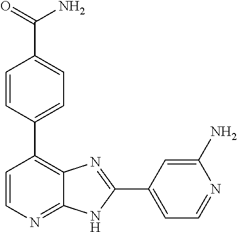

4-[2-(2-aminopyridin-4-yl)-3H-
imidazo[4,5-b]pyridin-7-yl]benzamide

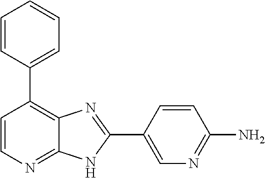

5-(7-phenyl-3H-imidazo[4,5-b]pyridin-2-
yl)pyridin-2-amine

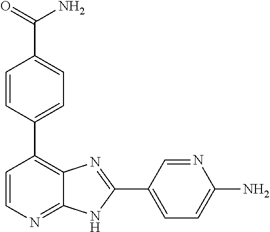

4-[2-(6-aminopyridin-3-yl)-3H-
imidazo[4,5-b]pyridin-7-yl]benzamide

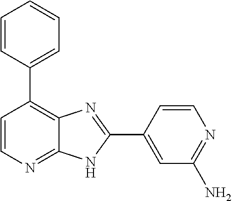

4-(7-phenyl-3H-imidazo[4,5-b]pyridin-2-
yl)pyridin-2-amine

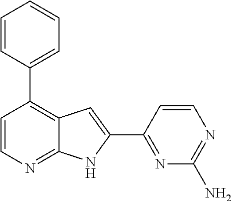

4-(4-phenyl-1H-pyrrolo[2,3-b]pyridin-2-
yl)pyrimidin-2-amine

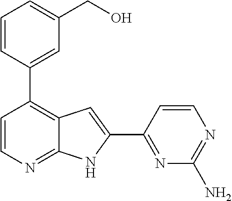

{3-[2-(2-aminopyrimidin-4-yl)-1H-
pyrrolo[2,3-b]pyridin-4-yl]phenyl}methanol

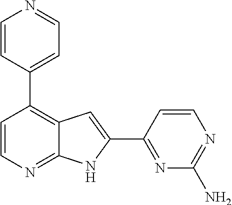

4-(4-pyridin-4-yl-1H-pyrrolo[2,3-
b]pyridin-2-yl)pyrimidin-2-amine

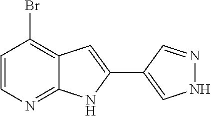

4-bromo-2-(1H-pyrazol-4-yl)-1H-
pyrrolo[2,3-b]pyridine

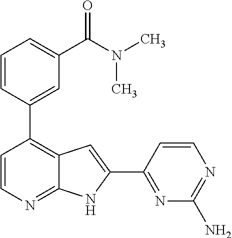

aminopyrimidin-4-yl)-1H-pyrrolo[2,3-
b]pyridin-4-yl]-N,N-dimethylbenzamide

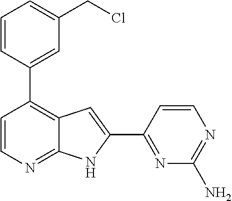

4-{4-[3-(chloromethyl)phenyl]-1H-
pyrrolo[2,3-b]pyridin-2-yl}pyrimidin-2-amine

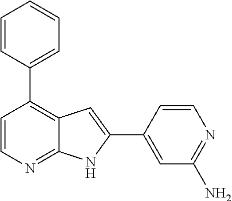

4-(4-phenyl-1H-pyrrolo[2,3-b]pyridin-2-
yl)pyridin-2-amine

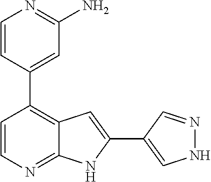

4-[2-(1H-pyrazol-4-yl)-1H-pyrrolo[2,3-
b]pyridin-4-yl]pyridin-2-amine

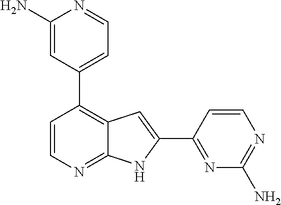

4-[4-(2-aminopyridin-4-yl)-1H-
pyrrolo[2,3-b]pyridin-2-yl]pyrimidin-2-amine

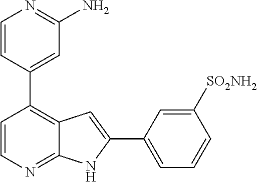

3-[4-(2-aminopyridin-4-yl)-1H-pyrrolo[2,3-
b]pyridin-2-yl]benzenesulfonamide

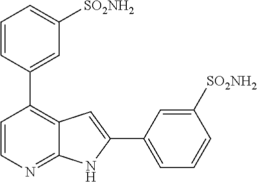

3,3′-(1H-pyrrolo[2,3-b]pyridine-2,4-
diyl)dibenzenesulfonamide

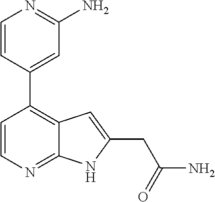

2-[4-(2-aminopyridin-4-yl)-1H-
pyrrolo[2,3-b]pyridin-2-yl]acetamide

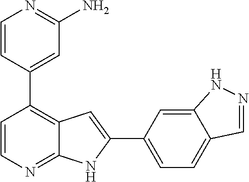

4-[2-(1H-indazol-6-yl)-1H-pyrrolo[2,3-
b]pyridin-4-yl]pyridin-2-amine

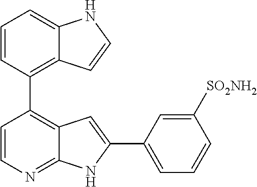

3-[4-(1H-indo1-4-yl)-1H-pyrrolo[2,3-
b]pyridin-2-yl]benzenesulfonamide

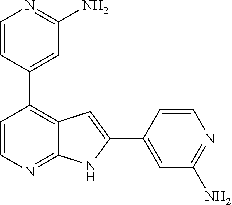

4,4′-(1H-pyrrolo[2,3-b]pyridine-2,4-
diyl)dipyridin-2-amine

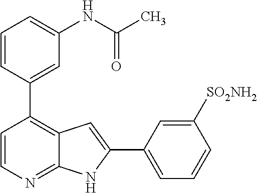

N-(3-{2-[3-(aminosulfonyl)phenyl]-1H-
pyrrolo[2,3-b]pyridin-4-yl}phenyl)acetamide

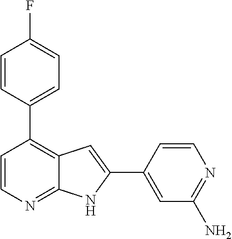

4-[4-(4-fluorophenyl)-1H-pyrrolo[2,3-
b]pyridin-2-yl]pyridin-2-amine

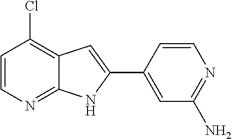

4-(4-chloro-1H-pyrrolo[2,3-b]pyridin-2-
yl)pyridin-2-amine

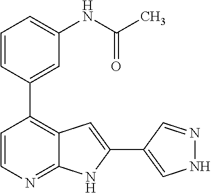

N-{3-[2-(1H-pyrazol-4-yl)-1H-pyrrolo[2,3-
b]pyridin-4-yl]phenyl}acetamide

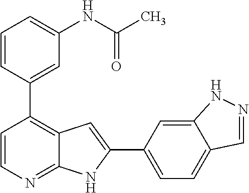

N-{3-[2-(1H-indazol-6-yl)-1H-pyrrolo[2,3-
b]pyridin-4-yl]phenyl}acetamide

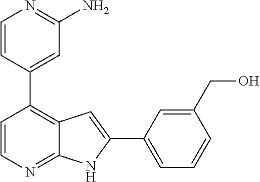

{3-[4-(2-aminopyridin-4-yl)-1H-
pyrrolo[2,3-b]pyridin-2-yl]phenyl}methanol

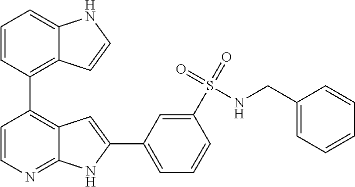

3-[4-(1H-indo1-4-yl)-1H-pyrrolo[2,3-b]pyridin-
2-yl]-N-(phenylmethyl)benzenesulfonamide

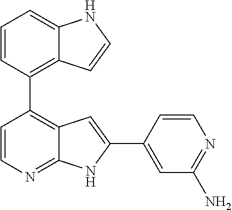

4-[4-(1H-indo1-4-yl)-1H-pyrrolo[2,3-
b]pyridin-2-yl]pyridin-2-amine

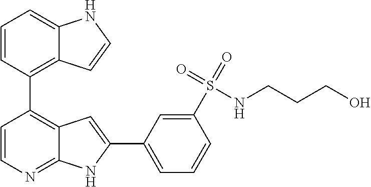

N-(3-hydroxypropyl)-3-[4-(1H-indo1-4-yl)-1H-
pyrrolo[2,3-b]pyridin-2-yl]benzenesulfonamide

TABLE 1C

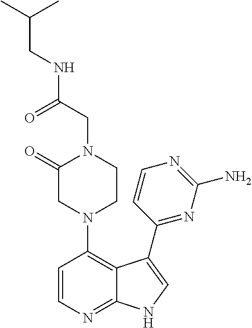

2-{4-[3-(2-aminopyrimidin-4-yl)-1H-pyrrolo[2,3-b]pyridin-
4-yl]-2-oxopiperazin-1-yl}-N-(2-methylpropyl)acetamide

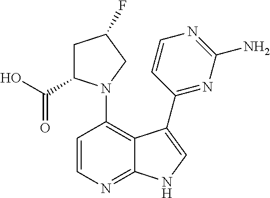

(4S)-1-[3-(2-aminopyrimidin-4-yl)-1H-
pyrrolo[2,3-b]pyridin-4-yl]-4-fluoro-L-proline

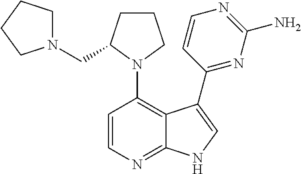

4-{4-[(2S)-2-(pyrrolidin-1-ylmethyl)pyrrolidin-
1-yl]-1H-pyrrolo[2,3-b]pyridin-3-yl}pyrimidin-2-amine

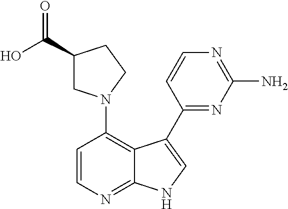

(3S)-1-[3-(2-aminopyrimidin-4-yl)-1H-
pyrrolo[2,3-b]pyridin-4-yl]pyrrolidine-3-carboxylic acid

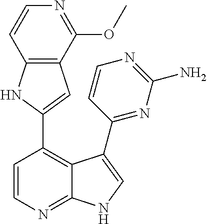

4-{4-[4-(methyloxy)-1H-pyrrolo[3,2-c]pyridin-2-yl]-
1H-pyrrolo[2,3-b]pyridin-3-yl}pyrimidin-2-amine

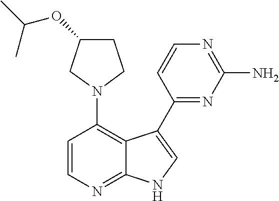

4-(4-{(3R)-3-[(1-methylethyl)oxy]pyrrolidin-1-yl}-
1H-pyrrolo[2,3-b]pyridin-3-yl)pyrimidin-2-amine

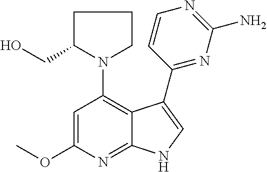

{(2S)-1-[3-(2-aminopyrimidin-4-yl)-6-(methyloxy)-1H-
pyrrolo[2,3-b]pyridin-4-yl]pyrrolidin-2-yl}methanol

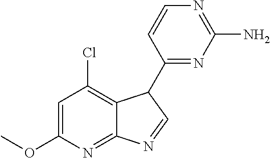

4-[4-chloro-6-(methyloxy)-3H-pyrrolo[2,3-
b]pyridin-3-yl]pyrimidin-2-amine

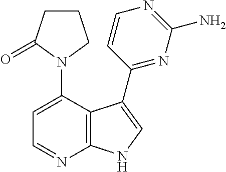

1-[3-(2-aminopyrimidin-4-yl)-1H-
pyrrolo[2,3-b]pyridin-4-yl]pyrrolidin-2-one

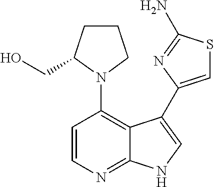

{(2S)-1-[3-(2-amino-1,3-thiazol-4-yl)-1H-
pyrrolo[2,3-b]pyridin-4-yl]pyrrolidin-2-yl}methanol

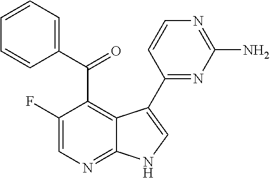

[3-(2-aminopyrimidin-4-yl)-5-fluoro-1H-
pyrrolo[2,3-b]pyridin-4-yl](phenyl)methanone

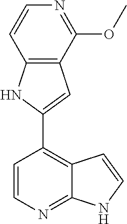

4-(methyloxy)-2-(1H-pyrrolo[2,3-
b]pyridin-4-yl)-1H-pyrrolo[3,2-c]pyridine

The compounds in TABLE !A, TABLE IB and TABLE IC each have CDK9 or CDK8 IC₅₀ values less than 6,000 nM. Another embodiment relates to compounds in TABLE IA and IB that CDK9 IC₅₀ values less than 1,000 nM. Another embodiment relates to compounds in TABLE IA and IB that CDK9 IC₅₀ values less than 500 nM. Another embodiment relates to compounds in TABLE IA and IB that CDK9 IC₅₀ values less than 100 nM. Another embodiment relates to compounds in TABLE IA and IB that CDK9 IC₅₀ values less than 50 nM. Another embodiment relates to compounds in TABLE IA and IB that CDK9 IC₅₀ values less than 25 nM. Another embodiment relates to compounds in TABLE IA and IB that CDK9 IC₅₀ values less than 10 nM.

Other embodiments relate to compounds from Table IA that fall with the scope of any of the embodiments above for Formula I, !A, IB, II, III, IV, V, VI or VII.

Other embodiments relate to compounds from Table IC that fall with the scope of any of the embodiments above for Formula I, !A, IB, II, III, IV, V, VI or VII.

Other embodiments relate to compounds from Table IB that fall with the scope of any of the embodiments above for Formula IC.

In another embodiment, the compound of Formula I is selected from one of the following compounds, or a pharmaceutically acceptable salt thereof:
4-[4-(4-ethylpiperazin-1-yl)-1H-pyrrolo[2,3-b]pyridin-3-yl]pyrimidin-2-amine;
3-(2-aminopyrimidin-4-yl)-N,N-diethyl-1H-pyrrolo[2,3-b]pyridin-4-amine;
3-(2-aminopyrimidin-4-yl)-N-(2-methylpropyl)-1H-pyrrolo[2,3-b]pyridin-4-amine;
4-[4-(hexahydrocyclopenta[c]pyrrol-2(1H)-yl)-1H-pyrrolo[2,3-b]pyridin-3-yl]pyrimidin-2-amine;
4-(methyloxy)-6-(4-piperidin-1-yl-1H-pyrrolo[2,3-b]pyridin-3-yl)pyrimidin-2-amine;
3-(2-aminopyrimidin-4-yl)-N-ethyl-1H-pyrrolo[2,3-b]pyridin-4-amine;
3-(2-aminopyrimidin-4-yl)-N-methyl-N-(1-methylpyrrolidin-3-yl)-1H-pyrrolo[2,3-b]pyridin-4-amine;
2-{1-[3-(2-aminopyrimidin-4-yl)-1H-pyrrolo[2,3-b]pyridin-4-yl]piperidin-3-yl}ethanol;
N-[3-(2-aminopyrimidin-4-yl)-1H-pyrrolo[2,3-b]pyridin-4-yl]-N,N′,N′-trimethylethane-1,2-diamine;
4-{4-[2-(aminomethyl)pyrrolidin-1-yl]-1H-pyrrolo[2,3-b]pyridin-3-yl}pyrimidin-2-amine;
{1-[3-(2-aminopyrimidin-4-yl)-1H-pyrrolo[2,3-b]pyridin-4-yl]pyrrolidin-2-yl}methanol;
4-(4-pyrrolidin-1-yl-1H-pyrrolo[2,3-b]pyridin-3-yl)pyrimidin-2-amine;
4-[4-(1-methylhexahydropyrrolo[3,4-b]pyrrol-5(1H)-yl)-1H-pyrrolo[2,3-b]pyridin-3-yl]pyrimidin-2-amine;
4-[4-(4-methylpiperazin-1-yl)-1H-pyrrolo[2,3-b]pyridin-3-yl]pyridin-2-amine;
4-{4-[3-(methylamino)pyrrolidin-1-yl]-1H-pyrrolo[2,3-b]pyridin-3-yl}pyrimidin-2-amine;
4-[4-(4-methylpiperazin-1-yl)-1H-pyrrolo[2,3-b]pyridin-3-yl]pyrimidin-2-amine;
ethyl 4-[3-(2-aminopyrimidin-4-yl)-1H-pyrrolo[2,3-b]pyridin-4-yl]piperazine-1-carboxylate;
4-(4-morpholin-4-yl-1H-pyrrolo[2,3-b]pyridin-3-yl)pyrimidin-2-amine;
4-{4-[(3S)-3-aminopyrrolidin-1-yl]-1H-pyrrolo[2,3-b]pyridin-3-yl}pyrimidin-2-amine;
4-[4-(5-methylhexahydropyrrolo[3,4-c]pyrrol-2(1H)-yl)-1H-pyrrolo[2,3-b]pyridin-3-yl]pyrimidin-2-amine;
4-[4-(4-methylpiperazin-1-yl)-1H-pyrrolo[2,3-b]pyridin-3-yl]pyridin-2-ol;
4-[4-(4-phenylpiperazin-1-yl)-1H-pyrrolo[2,3-b]pyridin-3-yl]pyrimidin-2-amine;
3-(2-aminopyrimidin-4-yl)-N-methyl-N-(1-methylethyl)-1H-pyrrolo[2,3-b]pyridin-4-amine;
3-(2-aminopyrimidin-4-yl)-N,N-dimethyl-1H-pyrrolo[2,3-b]pyridin-4-amine;
4-(5-phenyl-1H-pyrrolo[2,3-b]pyridin-3-yl)pyrimidin-2-amine;
4-[4-(3-aminopyrrolidin-1-yl)-1H-pyrrolo[2,3-b]pyridin-3-yl]pyrimidin-2-amine;
4-[4-(4-methylpiperidin-1-yl)-1H-pyrrolo[2,3-b]pyridin-3-yl]pyrimidin-2-amine;
4-[4-(3,5-dimethylpiperidin-1-yl)-1H-pyrrolo[2,3-b]pyridin-3-yl]pyrimidin-2-amine;
3-(2-aminopyrimidin-4-yl)-1H-pyrrolo[2,3-b]pyridin-5-amine;
4-(4-piperidin-1-yl-1H-pyrrolo[2,3-b]pyridin-3-yl)pyrimidin-2-amine;
N-{1-[3-(2-aminopyrimidin-4-yl)-1H-pyrrolo[2,3-b]pyridin-4-yl]pyrrolidin-3-yl}-N-methylacetamide;
4-methyl-6-[4-(4-methylpiperazin-1-yl)-1H-pyrrolo[2,3-b]pyridin-3-yl]pyrimidin-2-amine;
3-(2-aminopyrimidin-4-yl)-N-methyl-N-[2-(methyloxy)ethyl]-1H-pyrrolo[2,3-b]pyridin-4-amine;
4-{4-[4-(dimethylamino)piperidin-1-yl]-1H-pyrrolo[2,3-b]pyridin-3-yl}pyrimidin-2-amine;
4-[4-(4-aminopiperidin-1-yl)-1H-pyrrolo[2,3-b]pyridin-3-yl]pyrimidin-2-amine;
4-[4-(3,4,5-trimethylpiperazin-1-yl)-1H-pyrrolo[2,3-b]pyridin-3-yl]pyrimidin-2-amine;
6-(4-piperidin-1-yl-1H-pyrrolo[2,3-b]pyridin-3-yl)pyrimidine-2,4-diamine;
N-{1-[3-(2-aminopyrimidin-4-yl)-1H-pyrrolo[2,3-b]pyridin-4-yl]pyrrolidin-3-yl}acetamide;
4-(4-{(2R)-2-[(methyloxy)methyl]pyrrolidin-1-yl}-1H-pyrrolo[2,3-b]pyridin-3-yl)pyrimidin-2-amine;
3-(4-(piperidin-1-yl)-1H-pyrrolo[2,3-b]pyridin-3-yl)benzamide;
4-[4-(3-aminopiperidin-1-yl)-1H-pyrrolo[2,3-b]pyridin-3-yl]pyrimidin-2-amine;
3-(2-aminopyrimidin-4-yl)-N-methyl-N-(1-methylpiperidin-4-yl)-1H-pyrrolo[2,3-b]pyridin-4-amine;
3-(2-aminopyrimidin-4-yl)-N-methyl-N-(2-phenylethyl)-1H-pyrrolo[2,3-b]pyridin-4-amine;
(R)-(1-(3-(2-aminopyrimidin-4-yl)-1H-pyrrolo[2,3-b]pyridin-4-yl)pyrrolidin-2-yl)methanol;
4-[4-(3,5-dimethylpiperazin-1-yl)-1H-pyrrolo[2,3-b]pyridin-3-yl]pyrimidin-2-amine;
(3R)-1-[3-(2-aminopyrimidin-4-yl)-1H-pyrrolo[2,3-b]pyridin-4-yl]pyrrolidin-3-ol;
4-{4-[3-(dimethylamino)pyrrolidin-1-yl]-1H-pyrrolo[2,3-b]pyridin-3-yl}pyrimidin-2-amine;
(3S)-1-[3-(2-aminopyrimidin-4-yl)-1H-pyrrolo[2,3-b]pyridin-4-yl]pyrrolidin-3-ol;
4-(4-azepan-1-yl-1H-pyrrolo[2,3-b]pyridin-3-yl)pyrimidin-2-amine;
3-(2-aminopyrimidin-4-yl)-N,N-dipropyl-1H-pyrrolo[2,3-b]pyridin-4-amine;
4-{4-[3-(methyloxy)pyrrolidin-1-yl]-1H-pyrrolo[2,3-b]pyridin-3-yl}pyrimidin-2-amine;
4-{4-[(3R)-3-aminopyrrolidin-1-yl]-1H-pyrrolo[2,3-b]pyridin-3-yl}pyrimidin-2-amine;
4-(4-((1S,4S)-5-methyl-2,5-diazabicyclo[2.2.1]heptan-2-yl)-1H-pyrrolo[2,3-b]pyridin-3-yl)pyrimidin-2-amine;
N-methyl-4-(4-piperidin-1-yl-1H-pyrrolo[2,3-b]pyridin-3-yl)pyrimidin-2-amine;
5-(2-aminopyrimidin-4-yl)-N,N-dimethyl-7H-pyrrolo[2,3-d]pyrimidin-4-amine;
4-(4-(hexahydropyrrolo[1,2-a]pyrazin-2(1H)-yl)-1H-pyrrolo[2,3-b]pyridin-3-yl)pyrimidin-2-amine;
4-(4-{(2S)-2-[(methyloxy)methyl]pyrrolidin-1-yl}-1H-pyrrolo[2,3-b]pyridin-3-yl)pyrimidin-2-amine;
4-[4-(3,4-dimethylpiperazin-1-yl)-1H-pyrrolo[2,3-b]pyridin-3-yl]pyrimidin-2-amine;
4-(4-piperazin-1-yl-1H-pyrazolo[3,4-d]pyrimidin-3-yl)pyridin-2-amine;
{(2S)-1-[3-(2-aminopyrimidin-4-yl)-1H-pyrrolo[2,3-b]pyridin-4-yl]pyrrolidin-2-yl}methanol;
4-[4-(dimethylamino)-1H-pyrazolo[3,4-b]pyridin-3-yl]pyridin-2-ol;
4-[4-(dimethylamino)-1H-pyrazolo[3,4-d]pyrimidin-3-yl]phenol;
4-[4-(hexahydropyrrolo[3,4-b]pyrrol-1(2H)-yl)-1H-pyrrolo[2,3-b]pyridin-3-yl]pyrimidin-2-amine;
4-[4-(4-methyl-1,4-diazepan-1-yl)-1H-pyrrolo[2,3-b]pyridin-3-yl]pyrimidin-2-amine;
3-(2-aminopyrimidin-4-yl)-N,N-dimethyl-1H-pyrazolo[3,4-b]pyridin-4-amine;
4-[4-(3,3-dimethylpyrrolidin-1-yl)-1H-pyrrolo[2,3-b]pyridin-3-yl]pyrimidin-2-amine;
4-{4-[(3S)-3-fluoropyrrolidin-1-yl]-1H-pyrrolo[2,3-b]pyridin-3-yl}pyrimidin-2-amine;
1-[3-(2-aminopyrimidin-4-yl)-1H-pyrrolo[2,3-b]pyridin-4-yl]piperidin-4-ol;
N′-[3-(2-aminopyrimidin-4-yl)-1H-pyrrolo[2,3-b]pyridin-4-yl]-N,N-dimethylethane-1,2-diamine;
3-(2-aminopyridin-4-yl)-N,N-dimethyl-1H-pyrazolo[3,4-d]pyrimidin-4-amine;
3-(2-aminopyridin-4-yl)-N,N-dimethyl-1H-pyrazolo[3,4-b]pyridin-4-amine;
3-(2-aminopyrimidin-4-yl)-1H-pyrazolo[3,4-d]pyrimidin-4-amine; and
2-{[3-(2-aminopyrimidin-4.

Another aspect of this disclosure relates to a pharmaceutical composition comprising a compound according to Formula I, IA, IB, IC, II, III, IV, V, VI or VII, or a pharmaceutically acceptable salt thereof, and a pharmaceutically acceptable carrier, excipient or diluent.

Another aspect of this disclosure relates to a method of modulating CDK (ie., CDK1, CDK2, CDK4, CDK7 and/or CDK9) in a cell, comprising contacting a cell in which inhibition of CDK9 is desired with a compound according to Formula I, IA, IB, IC, II, III, IV, V, VI or VII, or a pharmaceutically acceptable salt thereof. In another embodiment of this aspect, the CDK is CDK9.

Another aspect of this disclosure relates to a method of inhibiting CDK (ie., CDK1, CDK2, CDK4, CDK7 and/or CDK9) in a cell, comprising contacting a cell in which inhibition of CDK9 is desired with a pharmaceutical composition comprising a compound according to Formula I, IA, IB, IC, II, III, IV, V, VI or VII, or a pharmaceutically acceptable salt thereof, and a pharmaceutically acceptable carrier, excipient or diluent. In another embodiment of this aspect, the CDK is CDK9.

Another aspect of this disclosure relates to a method of treating a disease or condition that involves CDK (ie., CDK1, CDK2, CDK4, CDK7 and/or CDK9) comprising administering to a patient, in need of the treatment, a compound according to Formula I, IA, IB, IC, II, III, IV, V, VI or VII, or a pharmaceutically acceptable salt thereof. Non-limiting examples of the disease or condition that can be treated in this embodiment include cancer such as lymphomas (particularly chronic lymphocytic lymphoma, Burkitts lymphoma and mantle cell lymphoma) multiple myeloma, breast cancer, small cell lung carcinoma and esophageal carcinoma. In another embodiment, disease or condition that can be treated include those related to HIV transcription and HTLV1 (which both require CDK9), such as HIV and HTLV associated leukemia. In another embodiment, disease or condition that can be treated include inflammatory diseases such as rheumatoid arthritis, multiple sclerosis, other auto immune diseases and transplant rejection. In another embodiment, disease or condition that can be treated include cardiac hypertrophy. In another embodiment of this aspect, the CDK is CDK9.

Another aspect of this disclosure relates to a method of treating a disease or condition that involves CDK (ie., CDK1, CDK2, CDK4, CDK7 and/or CDK9) comprising administering to a patient, in need of the treatment, a pharmaceutical composition comprising a compound according to Formula I, IA, IB, IC, II, III, IV, V, VI or VII, or a pharmaceutically acceptable salt thereof, and a pharmaceutically acceptable carrier, excipient or diluent. Non-limiting examples of the disease or condition that can be treated in this embodiment include cancer such as lymphomas (particularly chronic lymphocytic lymphoma, Burkitts lymphoma and mantle cell lymphoma) multiple myeloma, breast cancer, small cell lung carcinoma and esophageal carcinoma. In another embodiment, disease or condition that can be treated include those related to HIV transcription and HTLV1 (which both require CDK9), such as HIV and HTLV associated leukemia. In another embodiment, disease or condition that can be treated include inflammatory diseases such as rheumatoid arthritis, multiple sclerosis, other auto immune diseases and transplant rejection. In another embodiment, disease or condition that can be treated include cardiac hypertrophy. In another embodiment of this aspect, the CDK is CDK9.

Another aspect of this disclosure relates to a method of treating a disease or condition that involves CDK (ie., CDK1, CDK2, CDK4, CDK7 and/or CDK9) comprising administering to a patient, in need of the treatment, a compound according to Formula I, IA, IB, IC, II, III, IV, V, VI or VII, or a pharmaceutically acceptable salt thereof, in combination with radiation treatment and/or one or more therapeutic agents selected from Camptothecin, Topotecan, 9-Nitrocamptothecin, 9-Aminocamptothecin, Karenitecin, Irinotecan, Etoposide, Etoposide Phosphate, Teniposide, Amsacrine, Razoxane, Dexrazoxane, Mechlorethamine, Cyclophosphamide, Ifosfamide, Chlorambucil, Melphalan, Thiotepa, Trenimon, Triethylenemelamine, Rapamycin, Dianhydrogalactitol, Dibromodulcitol, Busulfan, dimethylsulfate, Chloroethylnitrosourea, BCNU, CCNU, Methyl-CCNU, Streptozotocin, Chlorozotocin, Prednimustine, Estramustine, Procarbazine, Dacarbazine, Hexamethylmelamine, Pentamethylmelamine, Temozolomide, Cisplatin, Carboplatin, Oxaliplatin, Bleomycin, Dactinomycin, Mithramycin, Mitomycin C, Daunorubicin, Doxorubicin, Epirubicin, Idarubicin, Methotrexate, Edatrexate, Trimethoprim, Nolatrexed, Raltitrexed, Hydroxyurea, 5-fluorouracil, Ftorafur, Capecitabine, Furtulon, Eniluracil, ara-C, 5-azacytidine, Gemcitabine, Mercaptopurine, Thioguanine, Pentostatin, antisense DNA, antisense RNA, an antisense DNA/RNA hybrid, a ribozyme, ultraviolet radiation, Vincristine, Vinblastine, Paclitaxel, Docetaxel, L-Asparaginase, a kinase inhibitor, Imatinib, Mitotane, Aminoglutethimide, Diethylstilbestrol, Ethinyl estradiol, Tamoxifen, Anastrozole, Testosterone propionate, Fluoxymesterone, Flutamide, Leuprolide, Prednisone, Hydroxyprogesterone caproate, Medroxyprogesterone acetate, Megestrol acetate, Interferon-alfa, and Interleukin. In a more specific embodiment, the combination is with Rapamycin. In another embodiment of this aspect, the CDK is CDK9.

Another aspect of this dislcosure relates to a method of treating a disease or condition that involves CDK (ie., CDK1, CDK2, CDK4, CDK7 and/or CDK9) comprising administering to a patient, in need of the treatment, a compound according to Formula I, IA, IB, IC, II, III, IV, V, VI or VII, or a pharmaceutically acceptable salt thereof, in combination with agents that induce apoptosis such as common chemotherapies like taxanes and platins, TNF related agents (TRAIL), bortezemib and TKIs. In another embodiment of this aspect, the CDK is CDK9.

Another aspect of this dislcosure relates to a pharmaceutical composition comprising a compound according to Formula IC, or a pharmaceutically acceptable salt thereof, and a pharmaceutically acceptable carrier, excipient or diluent.

Another aspect of this dislcosure relates to a method of inhibiting CDK (ie., CDK1, CDK2, CDK4, CDK7 and/or CDK9) in a cell, comprising contacting a cell in which inhibition of CDK is desired with a compound according to Formula IC, or a pharmaceutically acceptable salt thereof. In another embodiment of this aspect, the CDK is CDK9.

Another aspect of this dislcosure relates to a method of inhibiting CDK (ie., CDK1, CDK2, CDK4, CDK7 and/or CDK9) in a cell, comprising contacting a cell in which inhibition of CDK9 is desired with a pharmaceutical composition comprising a compound according to Formula IC, or a pharmaceutically acceptable salt thereof, and a pharmaceutically acceptable carrier, excipient or diluent. In another embodiment of this aspect, the CDK is CDK9.

Another aspect of this dislcosure relates to a method of treating a disease or condition that involves CDK (ie., CDK1, CDK2, CDK4, CDK7 and/or CDK9) comprising administering to a patient, in need of the treatment, a compound according to Formula IC, or a pharmaceutically acceptable salt thereof. Non-limiting examples of the disease or condition that can be treated in this embodiment include cancer such as lymphomas (particularly chronic lymphocytic lymphoma, Burkitts lymphoma and mantle cell lymphoma) multiple myeloma, breast cancer, small cell lung carcinoma and esophageal carcinoma. In another embodiment, disease or condition that can be treated include those related to HIV transcription and HTLV1 (which both require CDK9), such as HIV and HTLV associated leukemia. In another embodiment, disease or condition that can be treated include inflammatory diseases such as rheumatoid arthritis, multiple sclerosis, other auto immune diseases and transplant rejection. In another embodiment, disease or condition that can be treated include cardiac hypertrophy. In another embodiment of this aspect, the CDK is CDK9.

Another aspect of this dislcosure relates to a method of treating a disease or condition that involves CDK (ie., CDK1, CDK2, CDK4, CDK7 and/or CDK9) comprising administering to a patient, in need of the treatment, a pharmaceutical composition comprising a compound according to Formula IC, or a pharmaceutically acceptable salt thereof, and a pharmaceutically acceptable carrier, excipient or diluent. Non-limiting examples of the disease or condition that can be treated in this embodiment include cancer such as lymphomas (particularly chronic lymphocytic lymphoma, Burkitts lymphoma and mantle cell lymphoma) multiple myeloma, breast cancer, small cell lung carcinoma and esophageal carcinoma. In another embodiment, disease or condition that can be treated include those related to HIV transcription and HTLV1 (which both require CDK9), such as HIV and HTLV associated leukemia. In another embodiment, disease or condition that can be treated include inflammatory diseases such as rheumatoid arthritis, multiple sclerosis, other auto immune diseases and transplant rejection. In another embodiment, disease or condition that can be treated include cardiac hypertrophy. In another embodiment of this aspect, the CDK is CDK9.

Another aspect of this dislcosure relates to a method of treating a disease or condition that involves CDK (ie., CDK1, CDK2, CDK4, CDK7 and/or CDK9) comprising administering to a patient, in need of the treatment, a compound according to Formula IC, or a pharmaceutically acceptable salt thereof, in combination with radiation treatment and/or one or more therapeutic agents selected from Camptothecin, Topotecan, 9-Nitrocamptothecin, 9-Aminocamptothecin, Karenitecin, Irinotecan, Etoposide, Etoposide Phosphate, Teniposide, Amsacrine, Razoxane, Dexrazoxane, Mechlorethamine, Cyclophosphamide, Ifosfamide, Chlorambucil, Melphalan, Thiotepa, Trenimon, Triethylenemelamine, Rapamycin, Dianhydrogalactitol, Dibromodulcitol, Busulfan, dimethylsulfate, Chloroethylnitrosourea, BCNU, CCNU, Methyl-CCNU, Streptozotocin, Chlorozotocin, Prednimustine, Estramustine, Procarbazine, Dacarbazine, Hexamethylmelamine, Pentamethylmelamine, Temozolomide, Cisplatin, Carboplatin, Oxaliplatin, Bleomycin, Dactinomycin, Mithramycin, Mitomycin C, Daunorubicin, Doxorubicin, Epirubicin, Idarubicin, Methotrexate, Edatrexate, Trimethoprim, Nolatrexed, Raltitrexed, Hydroxyurea, 5-fluorouracil, Ftorafur, Capecitabin2e, Furtulon, Eniluracil, ara-C, 5-azacytidine, Gemcitabine, Mercaptopurine, Thioguanine, Pentostatin, antisense DNA, antisense RNA, an antisense DNA/RNA hybrid, a ribozyme, ultraviolet radiation, Vincristine, Vinblastine, Paclitaxel, Docetaxel, L-Asparaginase, a kinase inhibitor, Imatinib, Mitotane, Aminoglutethimide, Diethylstilbestrol, Ethinyl estradiol, Tamoxifen, Anastrozole, Testosterone propionate, Fluoxymesterone, Flutamide, Leuprolide, Prednisone, Hydroxyprogesterone caproate, Medroxyprogesterone acetate, Megestrol acetate, Interferon-alfa, and Interleukin. In a more specific embodiment, the combination is with Rapamycin. In another embodiment of this aspect, the CDK is CDK9.

Another aspect of this dislcosure relates to a method of treating a disease or condition that involves CDK (ie., CDK1, CDK2, CDK4, CDK7 and/or CDK9) comprising administering to a patient, in need of the treatment, a compound according to Formula IC, or a pharmaceutically acceptable salt thereof, in combination with agents that induce apoptosis such as common chemotherapies like taxanes and platins, TNF related agents (TRAIL), bortezemib and TKIs. In another embodiment of this aspect, the CDK is CDK9.

The following abbreviations and terms have the indicated meanings throughout:

| Abbreviation | Meaning |
|---|---|
| Ac | Acetyl |
| Br | Broad |
| ° C. | degrees Celsius |
| c- | Cyclo |
| CBZ | CarboBenZoxy = benzyloxycarbonyl |
| D | Doublet |
| Dd | doublet of doublet |
| Dt | doublet of triplet |
| DIPEA | N,N-diisopropylethylamine |
| DMF | N,N-dimethylformamide |
| DMSO | dimethyl sulfoxide |
| EI | Electron Impact ionization |
| Et | Ethyl |
| G | gram(s) |
| GC | gas chromatography |
| h or hr | hour(s) |
| HOAc | acetic acid |
| HOBt | Hydroxybenzotriazole |
| HPLC | high pressure liquid chromatography |
| L | liter(s) |
| M | molar or molarity |
| M | Multiplet |
| Me | Methyl |
| Mesyl | Methanesulfonyl |
| Mg | milligram(s) |
| MHz | Megahertz (frequency) |
| Min | minute(s) |
| mL | milliliter(s) |
| mM | Millimolar |
| Mmol | millimole(s) |
| Mol | mole(s) |
| MS | mass spectral analysis |
| MTBE | methyl t-butyl ether |
| N | normal or normality |
| NBS | N-bromosuccinimide |
| NCS | N-chlorosuccinimide |
| nM | Nanomolar |
| NMO | N-methylmorpholine oxide |
| NMR | nuclear magnetic resonance spectroscopy |
| PEG | polyethylene glycol |
| pEY | poly-glutamine, tyrosine |
| Ph | Phenyl |
| PhOH | Phenol |
| PfP | Pentafluorophenol |
| PfPy | Pentafluoropyridine |
| PPTS | Pyridinium p-toluenesulfonate |
| Py | Pyridine |
| PyBroP | bromo-tris-pyrrolidino-phosphonium hexafluorophosphate |
| Q | Quartet |
| RT | Room temperature |
| Sat'd | Saturated |
| S | Singlet |
| s- | Secondary |
| t- | Tertiary |
| t or tr | Triplet |
| TBDMS | t-butyldimethylsilyl |
| TES | Triethylsilyl |
| TFA | trifluoroacetic acid |
| THF | Tetrahydrofuran |
| TMOF | trimethyl orthoformate |
| TMS | Trimethylsilyl |
| Tosyl | p-toluenesulfonyl |
| Trt | triphenylmethyl |
| uL | microliter(s) |
| uM | Micromole(s) or micromolar |

As used in the present specification, the following words and phrases are generally intended to have the meanings as set forth below, except to the extert that the context in which they are used indicates otherwise or they are expressly defined to mean something different.

The symbol [symbol] means a single bond, [symbol] means a double bond, [symbol] means a triple bond, [symbol] means a single or double bond. When a group is depicted removed from its parent Formula, the [symbol] symbol will be used at the end of the bond which was theoretically cleaved in order to separate the group from its parent structural Formula.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual Formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

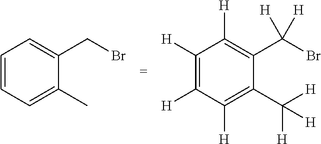

If a group “R” is depicted as “floating” on a ring system, as for example in the Formula:

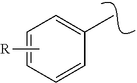

then, unless otherwise defined, a substituent “R” can reside on any atom of the ring system, assuming replacement of a depicted, implied, or expressly defined hydrogen from one of the ring atoms, so long as a stable structure is formed.

If a group “R” is depicted as floating on a fused ring system, as for example in the Formulae:

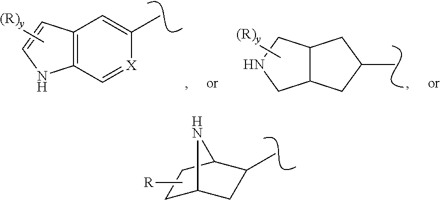

then, unless otherwise defined, a substituent “R” can reside on any atom of the fused ring system, assuming replacement of a depicted hydrogen (for example the —NH— in the Formula above), implied hydrogen (for example as in the Formula above, where the hydrogens are not shown but understood to be present), or expressly defined hydrogen (for example where in the Formula above, “X” equals ═CH—) from one of the ring atoms, so long as a stable structure is formed. In the example depicted, the “R” group can reside on either the 5-membered or the 6-membered ring of the fused ring system. In the Formula depicted above, when y is 2 for example, then the two “R's” can reside on any two atoms of the ring system, again assuming each replaces a depicted, implied, or expressly defined hydrogen on the ring.

When a group “R” is depicted as existing on a ring system containing saturated carbons, as for example in the Formula:

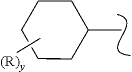

where, in this example, “y” can be more than one, assuming each replaces a currently depicted, implied, or expressly defined hydrogen on the ring; then, unless otherwise defined, where the resulting structure is stable, two “R's” can reside on the same carbon. A simple example is when R is a methyl group; there can exist a geminal dimethyl on a carbon of the depicted ring (an “annular” carbon). In another example, two R's on the same carbon, including that carbon, can form a ring, thus creating a spirocyclic ring (a “spirocyclyl” group) structure with the depicted ring as for example in the Formula:

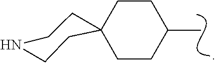

“Administration” and variants thereof (e.g., “administering” a compound) in reference to a compound of this disclosure (i.e., a compound of Formula I as described herein) means introducing the compound or a prodrug of the compound into the system of the animal in need of treatment. When a compound of this disclosure or prodrug thereof is provided in combination with one or more other active agents (e.g., surgery, radiation, and chemotherapy, etc.), “administration” and its variants are each understood to include concurrent and sequential introduction of the compound or prodrug thereof and other agents.

“Alkyl” is intended to include molecules having 1-12 carbons in size (C₁-C₁₂)alkyl, which can be straight chained or branched. For example, “C₆ alkyl” can refer to an n-hexyl, iso-hexyl, cyclobutylethyl, and the like. Alkyl is intended to include lower alkyl groups of from 1-6 carbons in size, such as methyl, ethyl, propyl, isopropyl, butyl, s-butyl, t-butyl, isobutyl, pentyl, hexyl and the like. An alkyl residue having a specific number of carbons is named, all geometric isomers having that number of carbons are intended to be encompassed; thus, for example, either “butyl” or “C₄ alkyl” is meant to include n-butyl, sec-butyl, isobutyl, t-butyl; and for example, “propyl” or “C₃ alkyl” each include n-propyl and isopropyl.

—(C₁-C₆)alkyl is a subset of alkyl groups that are from one to six carbon atoms in length, and can be straight chained or branched.

—(C₁-C₃)alkyl is a subset of alkyl groups that are from one to three carbon atoms in length, and can be straight chained or branched.

“Alkenyl” is intended to be an alkyl that contains at least one double bond between two carbons. Non-limiting examples of alkenyl include vinyl, allyl, isoprenyl, and the like.

“Alkynyl” is intended to be an alkyl that contains at least one triple bond between two carbons.

“Cycloalkyl” means a non-aromatic mono- or multicyclic ring system comprising about 3 to about 14 carbon atoms. Non-limiting examples of monocyclic cycloalkyls include cyclopropyl, cyclopentyl, cyclohexyl, cycloheptyl and the like. Non-limiting examples of multicyclic cycloalkyls include 1-decalin, norbornyl, adamantyl and the like. Cycloalkyls can be fused or bridge ring systems or spirocyclic systems.

“—(C₃-C₆)cycloalkyl” is a subset of cycloalkyl that means a non-aromatic monocyclic ring system comprising from 3 to 6 carbon atoms.

“Alkylene” refers to straight or branched chain divalent group consisting solely of carbon and hydrogen atoms, containing no unsaturation and having from one to ten carbon atoms, for example, methylene, ethylene, propylene, n-butylene and the like. Alkylene is a subset of alkyl, referring to the same residues as alkyl, but having two points of attachment and, specifically, fully saturated. Examples of alkylene include ethylene (—CH₂CH₂—), propylene (—CH₂CH₂CH₂—), dimethylpropylene (—CH₂C(CH₃)₂CH₂—), and cyclohexylpropylene (—CH₂CH₂CH(C₆H₁₃)).

“Alkoxy” or “alkoxyl” both refer to the group —O-alkyl, wherein the term “alkyl” is as defined above. Non-limiting examples of alkoxy include methoxy, ethoxy, propoxy, isopropoxy, and the like.

“—(C₁-C₆)alkoxy” is a subset of alkoxy that refers to the group —O—(C₁-C₆)alkyl, wherein the term “(C₁-C₆)alkyl” is as defined hereinabove.

“—(C₁-C₃)alkoxy” is a subset of alkoxy that refers to the group —O—(C₁-C₃)alkyl, wherein the term “(C₁-C₃)alkyl” is as defined hereinabove.

“Aryl” means a monovalent six- to ten-membered mono- or multicyclic ring, wherein the monocyclic ring is aromatic and at least one of the rings in the multicyclic ring is aromatic. A multicyclic ring that contains only one aryl ring is intended to be included within the definition of aryl. Representative non-limiting examples of aryl include phenyl, naphthyl, and the like.

“Arylalkyl” means a residue in which an aryl moiety, as defined above, is attached to a parent structure via one of an alkyl, wherein the alkyl portion is as defined above. Examples include benzyl, phenethyl, phenylvinyl, phenylallyl and the like. The “alkyl” portion of the group can be one to ten carbons.

“—(C₁-C₆)alkylaryl” is a subset of arylalkyl wherein the moiety is attached to a parent structure via a “—(C₁-C₆)alkylene group. Examples include benzyl, phenethyl, and the like.

In some examples, as appreciated by one of ordinary skill in the art, two adjacent groups on an aromatic system can be fused together to form a ring structure. The fused ring structure can contain heteroatoms and can be optionally substituted with one or more groups. It should additionally be noted that saturated carbons of such fused groups (i.e. saturated ring structures) can contain two substitution groups.

“Alkyl-C(O)N(H)(alkyl)-” refers to a monovalent group wherein the nitrogen atom of this group is bonded to the parent moiety, wherein the point of attachment is represented by the dash on the right hand side of this group, and the alkyl portions have the same meaning as the term “alkyl” as defined hereinabove.

“Fused-polycyclic” or “fused ring system” refers to a polycyclic ring system that contains bridged or fused rings; that is, where two rings have more than one shared atom in their ring structures. In this application, fused-polycyclics and fused ring systems includes non-aromatic and aromatic systems. Typically, but not necessarily, fused-polycyclics share a vicinal set of atoms, for example naphthalene or 1,2,3,4-tetrahydro-naphthalene. A spiro ring system is not a fused-polycyclic by this definition, but fused polycyclic ring systems of the compounds disclosed herein can themselves have spiro rings attached thereto via a single ring atom of the fused-polycyclic.

“Halogen” or “halo” both refer to fluorine, chlorine, bromine or iodine.

“Heteroatom” refers to O, S, N or P.

“Heterocycloalkyl” refers to a stable 4-12 membered monocyclic or multicyclic ring, wherein at least one of the rings contains at least one heteroatom and wherein there are no aromatic rings. Heterocycloalkyl is meant to include multicyclic rings, wherein one ring contains a heteroatom and another ring does not contain a heteroatom. Non-limiting examples of heterocycloalkyl include piperadinyl, piperazinyl, furanyl, pyrrolidinyl, morpholinyl.

“(4-6 membered) heterocycloalkyl” is a subset of heterocycloalkyl that refers to a stable 4-6 membered monocyclic ring containing at least one heteroatom and wherein there are no aromatic rings.

“Heterocycloalkylalkyl” refers to a heterocycloalkyl, as defined herein, attached to the parent moiety through an “alkyl,” wherein the alkyl portion is as defined above.

“Amino” refers to —NH₂.

“Alkylamino” refers to —NH(alkyl), wherein “alkyl” is as defined above, and wherein the parent moiety is attached to the nitrogen atom.

“Dialkylamino” refers to —N(alkyl)₂, wherein “alkyl” is as defined above, and wherein the parent moiety is attached to the nitrogen atom.

“Dialkylaminoalkyl” refers to -(alkyl)N(alkyl)₂, wherein “alkyl” is as defined above.

“Aminoalkyl” refers to -(alkyl)NH₂, wherein “alkyl” is as defined above, and wherein the parent moiety is attached to the alkyl group. The amino group can be attached at any point along the alkyl group.

“Heteroaryl” means a 5- to 12-membered, monocyclic aromatic heterocyclyl (where heterocyclyl is defined herein) or bicyclic heterocyclyl ring system (where at least one of the rings in the bicyclic system is aromatic) where the monocyclic ring and at least one of the rings in the bicyclic ring system contains one, two, three, four or five heteroatom(s) selected from nitrogen, oxygen, phosphorous, and sulfur. The ring containing the heteroatom can be aromatic or non-aromatic. Representative examples include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzdioxolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Fused, bridged, and spiro moieties are also included within the scope of this definition.

“(5-6 membered) Heteroaryl” means a 5 to 6-membered aromatic heterocyclyl ring system where the monocyclic ring and at least one of the contains one, two, three or four heteroatom(s) selected from nitrogen, oxygen, phosphorous, and sulfur. Representative examples include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl and pyrrolyl.

“Carbonyl” refers to the group “—C(O)-”, which is bivalent.

“Aminocarbonyl” refers to the group “—C(O)—NH₂,” wherein the parent moiety is attached to the carbonyl group.

“Alkoxycarbonyl” refers to the group “—C(O)alkoxy,” wherein alkoxy is as defined above, and the parent moiety is attached to the carbonyl. A non-limiting example includes —C(O)—OC(CH₃)₃.

“Hydroxyalkyl” refers to a group wherein the parent moiety is attached to the alkyl group, a hydroxyl group is attached to the alkyl, and the alkyl portion has the same meaning as the term “alkyl” as defined herein.

“Dihydroxyalkyl” refers to a group wherein the parent moiety is attached to the alkyl group, and a two hydroxyl groups are attached to the alkyl, wherein the alkyl portion is as defined in the term “alkyl” hereinabove.

“Alkylcarbonylamino” refers to the group “alkyl-C(O)—NH—,” wherein the parent moiety is attached to the amino (—NH—) group, and the alkyl portion has the same meaning as the term “alkyl” defined hereinabove.

In the case where there is a point of attachment for a monovalent substituent, such as —CH₃, —NH₂ or —OH, the indication of where the point of attachment is not necessary. That is, —CH₃ has the same meaning as CH₃, —NH₂ has the same meaning as NH₂, and —OH has the same meaning as OH.

In Table 1, where there appears to be an empty valence for oxygen or nitrogen for any of the compounds listed in this table, where the name of the structure requires that the empty valence is filled with hydrogen, it is assumed that the missing valence is filled with hydrogen for each of these cases.

When a group is referred to as “-(C₁-C₆)alkyl heterocyclyl” the heterocyclyl is attached to a parent structure via an alkyl group.

“Hydroxyalkyl” means -alkyl-OH, wherein alkyl is as defined hereinabove.

“Optional” or “optionally” means that the subsequently described event or circumstance can or can not occur, and that the description includes instances where said event or circumstance occurs and instances in which it does not. One of ordinary skill in the art would understand that with respect to any molecule described as containing one or more optional substituents, only sterically practical and/or synthetically feasible compounds are meant to be included. “Optionally substituted” means substituted or unsubstituted and refers to all subsequent modifiers in a term unless otherwise specified. So, for example, in the term “optionally substituted arylalkyl,” both the “alkyl” portion and the “aryl” portion of the molecule can be substituted or unsubstituted.

Unless otherwise specified, the term “optionally substituted” applies to the chemical moiety immediately preceding it. For instance, if a variable group (such as R) is defined as aryl, optionally substituted alkyl, or cycloalkyl, then only the alkyl group is optionally substituted.

“Saturated bridged ring system” refers to a bicyclic or polycyclic ring system that is not aromatic. Such a system can contain isolated or conjugated unsaturation, but not aromatic or heteroaromatic rings in its core structure (but can have aromatic substitution thereon). For example, hexahydro-furo[3,2-b]furan, 2,3,3a,4,7,7a-hexahydro-1H-indene, 7-aza-bicyclo[2.2.1]-heptane, and 1,2,3,4,4a,5,8,8a-octahydro-naphthalene are all included in the class “saturated bridged ring system.

“Spirocyclyl” or “spirocyclic ring” refers to a ring originating from a particular annular carbon of another ring. For example, as depicted below, a ring atom of a saturated bridged ring system (rings B and B′), but not a bridgehead atom, can be a shared atom between the saturated bridged ring system and a spirocyclyl (ring A) attached thereto. A spirocyclyl can be carbocyclic or heteroalicyclic.

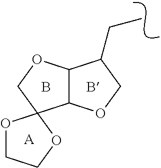

Some of the compounds of the disclosure can have imino, amino, oxo or hydroxy substituents off aromatic heterocyclyl systems. For purposes of this disclosure, it is understood that such imino, amino, oxo or hydroxy substituents can exist in their corresponding tautomeric form, i.e., amino, imino, hydroxy or oxo, respectively.

“Mammal” for the purposes of this disclosure includes humans (including patients receiving treatment) and other animals. Thus, the methods are applicable to both human therapy and veterinary applications. In a preferred embodiment, the mammal is a patient, and more preferably, the mammal is human.

“Therapeutically effective amount” is an amount of a compound of this disclosure, that when administered to a patient, ameliorates a symptom of the disease. The amount of a compound of this disclosure which constitutes a “therapeutically effective amount” will vary depending on the compound, the disease state and its severity, the age of the patient to be treated, and the like. The therapeutically effective amount can be determined routinely by one of ordinary skill in the art having regard to their knowledge and to this disclosure.

A “pharmaceutically acceptable salt” of a compound means a salt that is pharmaceutically acceptable and that possesses the desired pharmacological activity of the parent compound. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, Pa., 1985, which is incorporated herein by reference or S. M. Berge, et al., “Pharmaceutical Salts,” J. Pharm. Sci., 1977; 66:1-19 both of which are incorporated herein by reference.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

Examples of a pharmaceutically acceptable base addition salts include those formed when an acidic proton present in the parent compound is replaced by a metal ion, such as sodium, potassium, lithium, ammonium, calcium, magnesium, iron, zinc, copper, manganese, aluminum salts and the like. Preferable salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins. Examples of organic bases include isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, tromethamine, N-methylglucamine, polyamine resins, and the like. Exemplary organic bases are isopropylamine, diethylamine, ethanolamine, trimethylamine, dicyclohexylamine, choline, and caffeine.

All of the compounds disclosed herein include either their free base form or their pharmaceutically acceptable salts whether it is stated in the specification that these compounds can exist as their pharmaceutically acceptable salt or not.

“Prodrug” refers to compounds that are transformed (typically rapidly) in vivo to yield the parent compound of the above Formulae, for example, by hydrolysis in blood. Common examples include, but are not limited to, ester and amide forms of a compound having an active form bearing a carboxylic acid moiety. Examples of pharmaceutically acceptable esters of the compounds of this disclosure include, but are not limited to, alkyl esters (for example with between about one and about six carbons) the alkyl group is a straight or branched chain. Acceptable esters also include cycloalkyl esters and arylalkyl esters such as, but not limited to benzyl. Examples of pharmaceutically acceptable amides of the compounds of this disclosure include, but are not limited to, primary amides, and secondary and tertiary alkyl amides (for example with between about one and about six carbons). Amides and esters of the compounds of this disclosure can be prepared according to conventional methods. A thorough discussion of prodrugs is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Metabolite” refers to the break-down or end product of a compound or its salt produced by metabolism or biotransformation in the animal or human body; for example, biotransformation to a more polar molecule such as by oxidation, reduction, or hydrolysis, or to a conjugate (see Goodman and Gilman, “The Pharmacological Basis of Therapeutics” 8.sup.th Ed., Pergamon Press, Gilman et al. (eds), 1990 for a discussion of biotransformation). As used herein, the metabolite of a compound of this disclosure or its salt can be the biologically active form of the compound in the body. In one example, a prodrug can be used such that the biologically active form, a metabolite, is released in vivo. In another example, a biologically active metabolite is discovered serendipitously, that is, no prodrug design per se was undertaken. An assay for activity of a metabolite of a compound of this disclosure is known to one of skill in the art in light of the present disclosure.

The compounds of this disclosure also include N-oxide derivatives and protected derivatives of compounds of Formula I. For example, when compounds of Formula I contain an oxidizable nitrogen atom, the nitrogen atom can be converted to an N-oxide by methods well known in the art. When compounds of Formula I contain groups such as hydroxyl, carboxyl, thiol or any group containing a nitrogen atom(s), these groups can be protected with a suitable “protecting group” or “protective group”. A comprehensive list of suitable protective groups can be found in T. W. Greene, Protective Groups in Organic Synthesis, John Wiley & Sons, Inc. 1991, the disclosure of which is incorporated herein by reference in its entirety. The protected derivatives of compounds of Formula I can be prepared by methods well known in the art.

“Treating” or “treatment” of a disease, disorder or syndrome, as used herein, includes (i) preventing the disease, disorder or syndrome from occurring in a human, i.e. causing the clinical symptoms of the disease, disorder or syndrome not to develop in an animal that can be exposed to or predisposed to the disease, disorder or syndrome but does not yet experience or display symptoms of the disease, disorder or syndrome; (ii) inhibiting the disease, disorder or syndrome, i.e., arresting its development; and (iii) relieving the disease, disorder or syndrome, i.e., causing regression of the disease, disorder or syndrome. As is known in the art, adjustments for systemic versus localized delivery, age, body weight, general health, sex, diet, time of administration, drug interaction and the severity of the condition can be necessary, and will be ascertainable with routine experimentation by one of ordinary skill in the art.

One of ordinary skill in the art would understand that certain crystallized, protein-ligand complexes, in particular CDK9-ligand complexes, and their corresponding x-ray structure coordinates can be used to reveal new structural information useful for understanding the biological activity of kinases as described herein. As well, the key structural features of the aforementioned proteins, particularly, the shape of the ligand binding site, are useful in methods for designing or identifying selective modulators of kinases and in solving the structures of other proteins with similar features. Such protein-ligand complexes, having compounds of this disclosure as their ligand component, are an aspect of this disclosure.

As well, one of ordinary skill in the art would appreciate that such suitable x-ray quality crystals can be used as part of a method of identifying a candidate agent capable of binding to and modulating the activity of kinases. Such methods can be characterized by the following aspects: a) introducing into a suitable computer program, information defining a ligand binding domain of a kinase in a conformation (e.g. as defined by x-ray structure coordinates obtained from suitable x-ray quality crystals as described above) wherein the computer program creates a model of the three dimensional structures of the ligand binding domain, b) introducing a model of the three dimensional structure of a candidate agent in the computer program, c) superimposing the model of the candidate agent on the model of the ligand binding domain, and d) assessing whether the candidate agent model fits spatially into the ligand binding domain. Aspects a-d are not necessarily carried out in the aforementioned order. Such methods can further entail: performing rational drug design with the model of the three-dimensional structure, and selecting a potertial candidate agent in conjunction with computer modeling.

Additionally, one skilled in the art would appreciate that such methods can further entail: employing a candidate agent, so-determined to fit spatially into the ligand binding domain, in a biological activity assay for kinase modulation, and determining whether said candidate agent modulates kinase activity in the assay. Such methods can also include administering the candidate agent, determined to modulate kinase activity, to a mammal suffering from a condition treatable by kinase modulation, such as those described above.

Also, one skilled in the art would appreciate that compounds disclosed herein can be used in a method of evaluating the ability of a test agent to associate with a molecule or molecular complex comprising a ligand binding domain of a kinase. Such a method can be characterized by the following aspects: a) creating a computer model of a kinase binding pocket using structure coordinates obtained from suitable x-ray quality crystals of the kinase, b) employing computational algorithms to perform a fitting operation between the test agent and the computer model of the binding pocket, and c) analyzing the results of the fitting operation to quantify the association between the test agent and the computer model of the binding pocket.

### General Administration

In certain other preferred embodiments, administration can preferably be by the oral route. Administration of the compounds of this disclosure, or their pharmaceutically acceptable salts, in pure form or in an appropriate pharmaceutical composition, can be carried out via any of the accepted modes of administration or agents for serving similar utilities. Thus, administration can be, for example, orally, nasally, parenterally (intravenous, intramuscular or subcutaneous), topically, transdermally, intravaginally, intravesically, intracistemally or rectally, in the form of solid, semi-solid, lyophilized powder or liquid dosage forms, such as for example, tablets, suppositories, pills, soft elastic and hard gelatin capsules, powders, solutions, suspensions or aerosols, or the like, preferably in unit dosage forms suitable for simple administration of precise dosages.

The compositions will include a conventional pharmaceutical carrier or excipient and a compound of this disclosure as the/an active agent, and, in addition, can include carriers and adjuvants, etc.

Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It can also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the compounds in this disclosure can also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

The choice of Formulation depends on various factors such as the mode of drug administration (e.g., for oral administration, Formulations in the form of tablets, pills or capsules are preferred) and the bioavailability of the drug substance. Recently, pharmaceutical Formulations have been developed especially for drugs that show poor bioavailability based upon the principle that bioavailability can be increased by increasing the surface area i.e., decreasing particle size. For example, U.S. Pat. No. 4,107,288 describes a pharmaceutical Formulation having particles in the size range from 10 to 1,000 nm in which the active material is supported on a crosslinked matrix of macromolecules. U.S. Pat. No. 5,145,684 describes the production of a pharmaceutical Formulation in which the drug substance is pulverized to nanoparticles (average particle size of 400 nm) in the presence of a surface modifier and then dispersed in a liquid medium to give a pharmaceutical Formulation that exhibits remarkably high bioavailability.

Compositions suitable for parenteral injection can comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

One preferable route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms can also comprise buffering agents.

Solid dosage forms, as described above, can be prepared with coatings and shells, such as enteric coatings and others well known in the art. They can contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a compound(s) of this disclosure, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, can contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds of this disclosure with, for example, suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of a compound of this disclosure include ointments, powders, sprays, and inhalants. The active component is admixed under sterile conditions with a physiologically acceptable carrier and any preservatives, buffers, or propellants as can be required. Ophthalmic Formulations, eye ointments, powders, and solutions are also contemplated for the compounds in this disclosure.

Compressed gases can be used to disperse a compound of this disclosure in aerosol form. Inert gases suitable for this purpose are nitrogen, carbon dioxide, etc.

Generally, depending on the intended mode of administration, the pharmaceutically acceptable compositions will contain about 1% to about 99% by weight of a compound(s) of this disclosure, or a pharmaceutically acceptable salt thereof, and 99% to 1% by weight of a suitable pharmaceutical excipient. In one example, the composition will be between about 5% and about 75% by weight of a compound(s) of this disclosure, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of this disclosure, or a pharmaceutically acceptable salt thereof, for treatment of a disease-state in accordance with the teachings of this disclosure.

The compounds of this disclosure, or their pharmaceutically acceptable salts, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compounds of this disclosure can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

The compositions will include a conventional pharmaceutical carrier or excipient and a compound of this disclosure as the/an active agent, and, in addition, can include other medicinal agents and pharmaceutical agents. Compositions of the compounds in this disclosure can be used in combination with anticancer and/or other agents that are generally administered to a patient being treated for cancer, e.g. surgery, radiation and/or chemotherapeutic agent(s). Chemotherapeutic agents that can be useful for administration in combination with compounds of Formula I in treating cancer include alkylating agents, platinum containing agents.

If Formulated as a fixed dose, such combination products employ the compounds of this disclosure within the dosage range described above and the other pharmaceutically active agent(s) within its approved dosage range. Compounds of this disclosure can alternatively be used sequentially with known pharmaceutically acceptable agent(s) when a combination Formulation is inappropriate.

The examples and scheme below depict the general synthetic procedure for the compounds disclosed herein. Synthesis of the compounds disclosed herein is not limited by these examples and schemes. One skilled in the art will know that other procedures can be used to synthesize the compounds disclosed herein, and that the procedures described in the examples and schemes is only one such procedure. In the descriptions below, one of ordinary skill in the art would recognize that specific reaction conditions, added reagents, solvents, and reaction temperatures can be modified for the synthesis of specific compounds that fall within the scope of this disclosure.

Synthetic Procedures

The compounds disclosed herein, or their pharmaceutically acceptable salts, can have asymmetric carbon atoms, oxidized sulfur atoms or quaternized nitrogen atoms in their structure.

As stated above, all of the compounds disclosed herein include either their free base form or their pharmaceutically acceptable salts whether it is stated in the specification that these compounds can exist as their pharmaceutically acceptable salt or not. So, for instance, for any given embodiment of the compound of Formula I (including embodiments relating to the compounds themselves or method of use thereof), this embodiment includes either its free base form or any of its pharmaceutically acceptable salts, whether this is stated within this embodiment or not.

In addition, all of the compounds disclosed herein, including any of their pharmaceutically acceptable salts, can exist as single stereoisomers (including single enantiomres and single diastereomers), racemates, mixtures of enantiomers and diastereomers and polymorphs. Sterioisomers of the compounds in this disclosure include geometric isomers and optical isomers, such as atropisomers. The compounds disclosed herein can also exist as geometric isomers. All such single stereoisomers, racemates and mixtures thereof, and geometric isomers are intended to be within the scope of the compounds disclosed herein.

It is assumed that when considering generic descriptions of compounds of the disclosed herein for the purpose of constructing a compound, such construction results in the creation of a stable structure. That is, one of ordinary skill in the art would recognize that theoretically some constructs which would not normally be considered as stable compounds (that is, sterically practical and/or synthetically feasible, supra).

Methods for the preparation and/or separation and isolation of single stereoisomers from racemic mixtures or non-racemic mixtures of stereoisomers are well known in the art. For example, optically active (R)- and (S)-isomers can be prepared using chiral synthons or chiral reagents, or resolved using conventional techniques. Enantiomers (R- and S-isomers) can be resolved by methods known to one of ordinary skill in the art, for example by: formation of diastereoisomeric salts or complexes which can be separated, for example, by crystallization; via formation of diastereoisomeric derivatives which can be separated, for example, by crystallization, selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step can be required to liberate the desired enantiomeric form. Alternatively, specific enantiomer can be synthesized by asymmetric synthesis using optically active reagents, substrates, catalysts or solvents or by converting on enantiomer to the other by asymmetric transformation. For a mixture of enantiomers, enriched in a particular enantiomer, the major component enantiomer can be further enriched (with concomitant loss in yield) by recrystallization.

In addition, the compounds of this disclosure can exist in unsolvated as well as solvated forms with pharmaceutically acceptable solvents such as water, ethanol, and the like. In general, the solvated forms are considered equivalent to the unsolvated forms for the purposes of the compounds of this disclosure.

In addition, it is intended that the present disclosure cover compounds made either using standard organic synthetic techniques, including combinatorial chemistry or by biological methods, such as bacterial digestion, metabolism, enzymatic conversion, and the like.

The examples and scheme below depict the general synthetic procedure for the compounds disclosed herein. Synthesis of the compounds disclosed herein is not limited by these examples and schemes. One skilled in the art will know that other procedures can be used to synthesize the compounds disclosed herein, and that the procedures described in the examples and schemes is only one such procedure. In the descriptions below, one of ordinary skill in the art would recognize that specific reaction conditions, added reagents, solvents, and reaction temperatures can be modified for the synthesis of specific compounds that fall within the scope of this disclosure.

The terms “modulate”, “modulation” and the like refer to the ability of a compound to increase or decrease the function, or activity of, for example, CDK9. “Modulation”, as used herein in its various forms, is intended to encompass inhibition, antagonism, partial antagonism, activation, agonism and/or partial agonism of the activity associated with CDK9. CDK9 inhibitors are compounds that, e.g., bind to, partially or totally block stimulation, decrease, prevent, delay activation, inactivate, desensitize, or down regulate signal transduction. CDK9 activators are compounds that, e.g., bind to, stimulate, increase, open, activate, facilitate, enhance activation, sensitize or up regulate signal transduction. The ability of a compound to modulate CDK9 can be demonstrated in an enzymatic assay or a cell-based assay. For example, the inhibition of CDK9 may decrease cortisol levels in a patient and/or increase cortisone levels in a patient by blocking the conversion of cortisone to cortisol.

As used herein, the term “CDK9-mediated condition or disorder” and related terms and phrases refer to a condition or disorder characterized by inappropriate, e.g., less than or greater than normal, activity of CDK9. A CDK9-mediated condition or disorder may be completely or partially characterized by inappropriate CDK9 activity. However, a CDK9-mediated condition or disorder is one in which modulation of a CDK9 results in some effect on the underlying condition or disease (e.g., a CDK9 inhibitor results in some improvement in patient well-being in at least some patients).

As used herein, the term “CDK9-mediated condition or disorder” and related terms and phrases refer to a condition or disorder characterized by inappropriate, e.g., less than or greater than normal, CDK9 activity. An CDK9-mediated condition or disorder may be completely or partially characterized by inappropriate CDK9 activity. However, an CDK9-mediated condition or disorder is one in which modulation of CDK9 results in some effect on the underlying condition or disease (e.g., a CDK9 inhibitor results in some improvement in patient well-being in at least some patients).

The examples and schemes below depict the general synthetic procedure for the compounds disclosed herein. Synthesis of the compounds disclosed herein is not limited by these examples and schemes. One skilled in the art will know that other procedures can be used to synthesize the compounds disclosed herein, and that the procedures described in the examples and schemes is only one such procedure. In the descriptions below, one of ordinary skill in the art would recognize that specific reaction conditions, added reagents, solvents, and reaction temperatures can be modified for the synthesis of specific compounds that fall within the scope of this disclosure.

### Synthesis of Compounds

### Synthetic Procedures

The compounds disclosed herein, or their pharmaceutically acceptable salts, can have asymmetric carbon atoms, oxidized sulfur atoms or quaternized nitrogen atoms in their structure.

The compounds disclosed herein and their pharmaceutically acceptable salts can exist as single stereoisomers, racemates, and as mixtures of enantiomers and diastereomers. The compounds disclosed herein can also exist as geometric isomers. All such single stereoisomers, racemates and mixtures thereof, and geometric isomers are intended to be within the scope of the compounds disclosed herein.

It is assumed that when considering generic descriptions of compounds of the disclosed herein for the purpose of constructing a compound, such construction results in the creation of a stable structure. That is, one of ordinary skill in the art would recognize that theoretically some constructs which would not normally be considered as stable compounds (that is, sterically practical and/or synthetically feasible, supra).

Methods for the preparation and/or separation and isolation of single stereoisomers from racemic mixtures or non-racemic mixtures of stereoisomers are well known in the art. For example, optically active (R)- and (S)-isomers can be prepared using chiral synthons or chiral reagents, or resolved using conventional techniques. Enantiomers (R- and S-isomers) can be resolved by methods known to one of ordinary skill in the art, for example by: formation of diastereoisomeric salts or complexes which can be separated, for example, by crystallization; via formation of diastereoisomeric derivatives which can be separated, for example, by crystallization, selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step can be required to liberate the desired enantiomeric form. Alternatively, specific enantiomer can be synthesized by asymmetric synthesis using optically active reagents, substrates, catalysts or solvents or by converting on enantiomer to the other by asymmetric transformation. For a mixture of enantiomers, enriched in a particular enantiomer, the major component enantiomer can be further enriched (with concomitant loss in yield) by recrystallization.

In addition, the compounds of this disclosure can exist in unsolvated as well as solvated forms with pharmaceutically acceptable solvents such as water, ethanol, and the like. In general, the solvated forms are considered equivalent to the unsolvated forms for the purposes of the compounds of this disclosure.

In addition, it is intended that the present disclosure cover compounds made either using standard organic synthetic techniques, including combinatorial chemistry or by biological methods, such as bacterial digestion, metabolism, enzymatic conversion, and the like.

The examples and scheme below depict the general synthetic procedure for the compounds disclosed herein. Synthesis of the compounds of Formulae I, IA, IB, II, III, IV, V, VI, and VII disclosed herein are not limited by these examples and schemes. One skilled in the art will know that other procedures can be used to synthesize the compounds of Formulae I, IA, IB, II, III, IV, V, VI, and VII disclosed herein, and that the procedures described in the examples and schemes is only one such procedure. In the descriptions below, one of ordinary skill in the art would recognize that specific reaction conditions, added reagents, solvents, and reaction temperatures can be modified for the synthesis of specific compounds that fall within the scope of this disclosure. All intermediate compounds described below, for which there is no description of how to synthesize such intermediates within these examples below, are commercially available compounds unless otherwise specified.

### EXAMPLES

### Instrumentation

IR spectra were collected by reflectance on a Perkin Elmer Spectrum™100 FT-IR. ¹H NMR were collected on a Varian 400 MHz with Mercury and Mercury consoles.

### Example 1

### 4-(4-chloro-1H-pyrrolo[2,3-b]pyridin-3-yl)pyrimidin-2-amine

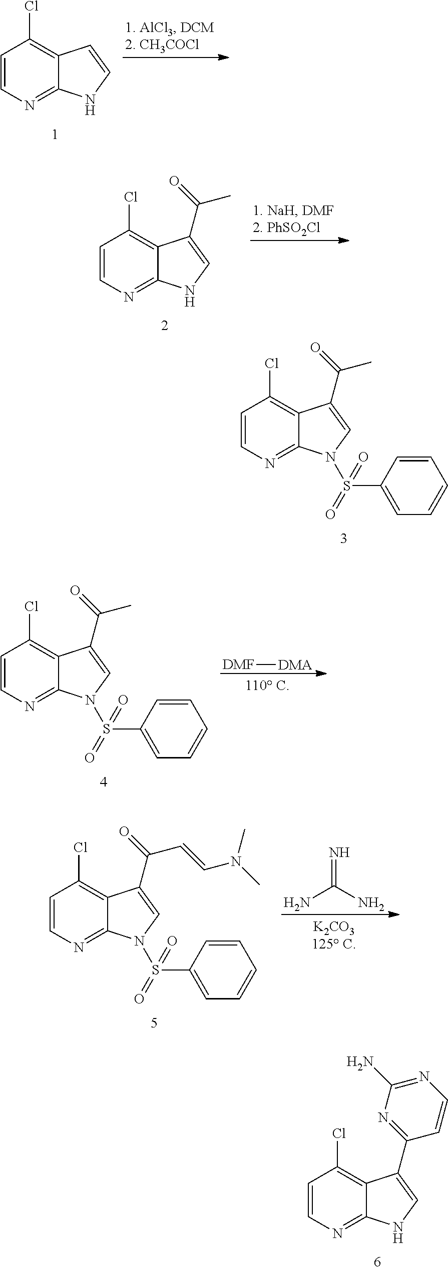

### 3-acetyl-4-chloro-7-azaindol

To a solution of aluminum chloride (4.4 g, 33 mmol) in dichloromethane (50 mL) was added from commercially available 4-chloro-7-azaindole 1 (1 g, 6.6 mmol). The mixture was stirred at room temperature for 1 hour. Acetyl chloride (2.4 mL, 33 mmol) was added dropwise and the resulting mixture was stirred overnight. 10 mL of methanol was then added slowly. The solvent was removed in vacuo and water was added. The mixture was subjected to sonication for 30 minutes. The precipitate was collected by filtration, rinsed with water, and dried (1.05 g, 82%). The solid 3-acetyl-4-chloro-7-azaindol 2 was used for the next step without further purification. MS (EI) for C₉H₈ClN₂O, found 195 (MH+).

### 1-(4-chloro-1-(phenylsulfonyl)-1H-pyrrolo[2,3-b]pyridine-3-yl)ethanone

Sodium hydride (0.35 g, 8.6 mmol, 60%) was added to the solution of 3-acetyl-4-chloro-7-azaindol 2 (1.05 g, 5.4 mmol) in THF and DMF (1:1, 20 mL). The mixture was stirred for 10 minutes and benzene sulfonyl chloride (1.4 mL, 10.8 mmol) was added. The resulting solution was stirred at room temperature for 1 hour. 10 mL of a saturated ammonium chloride solution was then slowly added. The mixture was extracted with ethyl acetate and the resulting organic extracts were concentrated under reduced pressure. The crude mixture was purified via flash chromatography to afford 1-(4-chloro-1-(phenylsulfonyl)-1H-pyrrolo[2,3-b]pyridine-3-yl)ethanone 4 (0.8 g, 44%). MS (EI) for C₁₈H₁₂ClN₂O₃S, found 334.9 (MH+).

### (E)-1-(4-chloro-1-(phenylsulfonyl)-1H-pyrrolo[2,3-b]pyridin-3-yl)-3-(dimethylamino)prop-2-en-1-one

1-(4-chloro-1-(phenylsulfonyl)-1H-pyrrolo[2,3-b]pyridin-3-yl)ethanone 4 and N,N-dimethyl formamide dimethyl acetal (DMF-DMA) were mixed in 10 mL of toluene. The mixture was heated at reflux overnight. Additional DMF-DMA was added to the solution and reflux continued for an additional 24 hours. Toluene and the DMF-DMA were removed in vacuo, and the resulting (E)-1-(4-chloro-1-(phenylsulfonyl)-1H-pyrrolo[2,3-b]pyridin-3-yl)-3-(dimethylamino)prop-2-en-1-one 5 was used in the next step without further purification. MS (EI) for C₁₈H₁₇ClN₃O₃S, found 390 (MH+).

### 4-(4-chloro-1H-pyrrolo[2,3-b]pyridin-3-yl)pyrimidin-2-amine

(E)-1-(4-chloro-1-(phenylsulfonyl)-1H-pyrrolo[2,3-b]pyridin-3-yl)-3-(dimethylamino)prop-2-en-1-one 5 (52 mg, 13 mmol), guanidine hydrochloride (18 mg, 20 mmol) and potassium carbonate (36 mg, 26 mmol) were added to 3 mL of 2-methoxyethanol. The mixture was heated at reflux overnight. The crude mixture was purified by preparative HPLC to afford 4-(4-chloro-1H-pyrrolo[2,3-b]pyridin-3-yl)pyrimidin-2-amine 6 (14 mg, 46%). ¹H-NMR (400 MHz, CDCl₃): δ 8.28 (m, 2H), 8.2 (d, 1H), 7.34 (m, 2H). MS (EI) for C₁₁H₉ClN₈, found 246 (MH+).

The following compounds were made in a manner analogous to Example 1:

### Example 2

### 4-(5-bromo-1H-pyrrolo[2,3-b]pyridin-3-yl)pyrimidin-2-amine

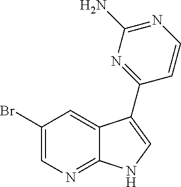

This compound was made using the analogous procedure as described for Example 1 but starting from commercially available 5-bromo-7-azaindol in place of 4-chloro-7-azaindol. ¹H NMR (400 MHz, DMSO-d₆): δ 9.09 (d, 1H), 8.41 (s, 1H), 8.33 (d, 1H), 8.11 (d, 1H), 7.04 (d, 1H), 6.59 (br s, 2H). MS (EI) for C₁₁H₉BrN₅, found 289.9/291.9 (MH+).

### Example 3

### 4(5-phenyl-1H-pyrrolo[2,3-b]pyridin-3-yl)pyrimidin-2-amine

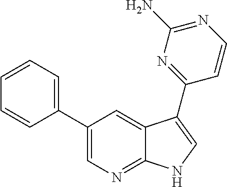

This compound was made using the analogous procedure as described for Example 2. Following formation of 1-(5-bromo-1-(phenylsulfonyl)-1H-pyrrolo[2,3-b]pyridin-3-yl)ethanone, the crude material was stirred with phenyl boronic acid, Pd(dppf)Cl₂ and potassium carbonate in DME in a Suzuki procedure similar to Example 22. The resulting residue was purified via flash chromatography to afford 1-(5-phenyl-1-(phenylsulfonyl)-1H-indol-3-yl)ethanone and the subsequent synthesis was completed in an analogous manner as Example 1 to afford 4-(5-phenyl-1H-pyrrolo[2,3-b]pyridin-3-yl)pyrimidin-2-amine. ¹H-NMR (400 MHz, DMSO-d₆): δ 9.09 (d, 1H), 8.41 (s, 1H), 8.33 (d, 1H), 8.11 (d, 1H), 7.04 (d, 1H), 6.59 (br s, 2H). MS (EI) for C₁₁H₉BrN₅, found 289.9/291.9 (MH+).

### Representative Conditions under Scheme 2

### Example 4

### 4(4-(4-methylpiperzin-1-yl)-1H-pyrrolo[2,3-b]pyridin-3-yl)pyrimidin-2-amine

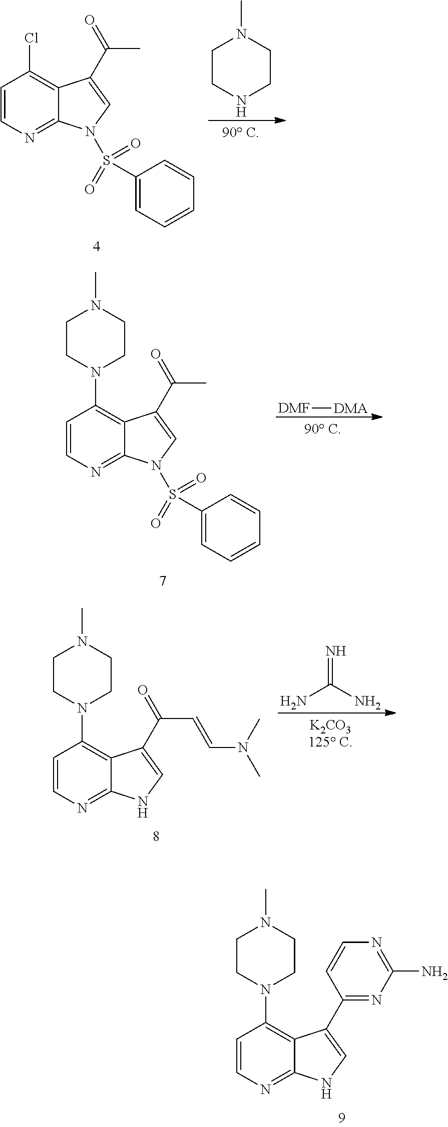

1-(4-(4-methylpiperizin-1-yl)-1-(phenylsulfonyl)-1H-pyrrolo[2,3-b]pyridin-3-yl)ethanone

1-(4-chloro-1-(phenylsulfonyl)-1H-pyrrolo[2,3-b]pyridin-3-yl)ethanone 4 (0.2 g, 0.6 mmol) was added to 1 mL of N-methylpiperazine and the mixture was stirred at 90° C. for 30 minutes. Excess amine was removed in vacuo to afford 1-(4-(4-methylpiperizin-1-yl)-1-(phenylsulfonyl)-1H-pyrrolo[2,3-b]pyridin-3-yl)ethanone 7. MS (EI) for C₂₀H₂₃N₄O₃, found 399 (MH+).

### (E)-1-(4-(4-methylpiperizin-1-yl)-1-(phenylsulfonyl)-1H-pyrrolo[2,3-b]pyridin-3-yl)-3-(dimethylamino)prop-2-en-1-one

1-(4-(4-methylpiperizin-1-yl)-1-(phenylsulfonyl)-1H-pyrrolo[2,3-b]pyridin-3-yl)ethanone 7 (0.23 g, 0.6 mmol) was added to 10 mL of DMF/DMA, and the mixture was stirred at 90° C. overnight. Excess DMF/DMA was removed in vacuo. The resulting solid (E)-1-(4-(4-methylpiperizin-1-yl)-1-(phenylsulfonyl)-1H-pyrrolo[2,3-b]pyridin-3-yl)-3-(dimethylamino)prop-2-en-1-one 8 was washed with ether and used in the next step without further purification. MS (EI) for C₁₇H₂₄N₅O, found 314 (MH+).

### 4-(4-(4-methylpiperzin-14)-1H-pyrrolo[2,3-b]pyridin-3-yl)pyrimidin-2-amine

(E)-1-(4-morphilino-1-(phenylsulfonyl)-1H-pyrrolo[2,3-b]pyridin-3-yl)-3-(dimethylamino)prop-2-en-1-one 8 (0.19 g, 0.6 mmol), guanidine (0.12 g, 1.2 mmol) and potassium carbonate (0.33 g, 2.4 mmol) were added to 5 mL of 2-methoxyethanol. The mixture was refluxed at 125° C. overnight. The crude mixture was purified by preparative HPLC to afford 4-(4-(4-methylpiperzin-1-yl)-1H-pyrrolo[2,3-b]pyridin-3-yl)pyrimidin-2-amine 9 (60 mg, 32%). ¹H-NMR (400 MHz, DMSO): δ 12.00 (s, 1H), 8.23 (d, 1H), 8.10 (d, 1H), 7.75 (s, 1H), 7.00 (d, 1H), 6.69 (d, 1H), 6.44 (s, 2H), 2.98 (s, 4H), 2.37 (s, 4H), 2.18 (s, 3H). MS (EI) for C₁₆H₂₀N₇, found 310 (MH+).

The following compounds were made in a manner analogous to Example 4:

### Example 5

### 4-(4-(4-methylpiperzin-14)-1H-pyrrolo[2,3-b]pyridin-3-yl)pyrimidin-2-amine

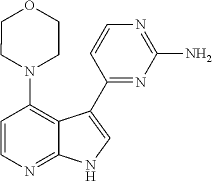

The same procedure in Example 4 was used to synthesize the title compound wherein morpholine was substituted for 4-methylpiperizine. ¹H-NMR (400 MHz, DMSO): δ 12.3 (s, 1H), 8.23 (d, 1H), 8.10 (d, 1H), 7.75 (s, 1H), 7.00 (d, 1H), 6.69 (d, 1H), 6.44 (s, 2H), 3.62 (s, 4H), 2.95 (s, 4H). MS (EI) for C₁₅H₁₇N₆O, found 297 (MH+).

### Example 6

### 3-(2-aminopyrimidin-4-yl)-N,N-dimethyl-1H-pyrrolo[2,3-b]pyridin-4-amine

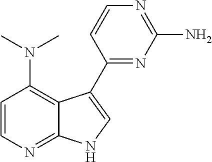

The same procedure in Example 4 was used to synthesize the title compound wherein a methanolic solution of dimethylamine was substituted for 4-methylpiperizine. ¹H-NMR (400 MHz, DMSO): δ 11.80 (s, 1H), 8.18 (d, 1H), 8.05 (d, 1H), 7.73 (s, 1H), 6.92 (d, 1H), 6.61 (d, 1H), 6.4 (s, 2H), 2.68 (s, 6H). MS (EI) for C₁₃H₁₅N₆, found 255 (MH+).

### Example 7

### 4-[4-piperidin-1-yl-1H-pyrrolo[2,3-b]pyridin-3-yl]pyrimidin-2-amine

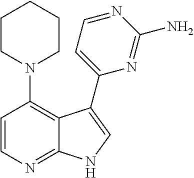

The same procedure in Example 4 was used to synthesize the title compound wherein piperidine was substituted for the amine ¹H-NMR (400 MHz, DMSO-d₆): δ 12.00 (s, 1H), 8.23 (d, 1H), 8.08 (d, 1H), 7.73 (s, 1H), 7.03 (d, 1H), 6.68 (d, 1H), 6.42 (s, 2H), 2.94 (m, 4H), 1.52 (m, 4H), 1.47 (m, 2H). MS (ES) for C₁₆H₁₉N₆, found 295.1 (MH+).

### Example 8

### 3-(2-aminopyrimidin-4-yl)-N,N-diethyl-1H-pyrrolo[2,3-b]pyridin-4-amine

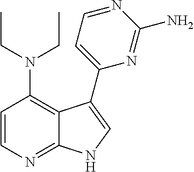

The same procedure in Example 4 was used to synthesize the title compound wherein a methanolic solution of diethylamine was substituted for the amine ¹H-NMR (400 MHz, DMSO-d₆): δ 8.16 (d, 1H), 8.06 (d, 1H), 7.75 (s, 1H), 7.06 (d, 1H), 6.68 (d, 1H), 6.37 (s, 2H), 3.12 (q, 4H), 0.92 (t, 6H). MS (ES) for C₁₅H₁₉N₆, found 283.1 (MH+).

### Example 9

### 4-(4-azepan-1-yl-1H-pyrrolo[2,3-b]pyridin-3-yl)pyrimidin-2-amine

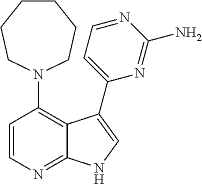

The same procedure in Example 4 was used to synthesize the title compound wherein azepane was substituted for the amine. ¹H-NMR (400 MHz, DMSO-d₆): δ 11.90 (s, 1H), 8.15 (d, 1H), 7.98 (d, 1H), 7.62 (s, 1H), 6.76 (d, 1H), 6.62 (d, 1H), 6.41 (s, 2H), 3.32 (m, 4H), 1.52 (m, 8H). MS (ES) for C₁₇H₂₁N₆, found 309.1 (MH+).

### Example 10

### 4-(4-(3-aminopiperidin-1-yl)-1H-pyrrolo[2,3-b]pyridin-3-yl)pyrimidin-2-amine

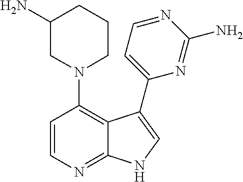

The same procedure in Example 4 was used to synthesize the title compound wherein 3-aminopiperidine was substituted for the amine. ¹H-NMR (400 MHz, DMSO-d₆): δ 12.12 (s, 1H), 8.20 (d, 1H), 8.08 (d, 1H), 7.72 (s, 1H), 6.96 (d, 1H), 6.66 (d, 1H), 6.41 (s, 2H), 3.19 (m, 1H), 2.78 (m, 1H), 2.46 (m, 1H), 2.32 (m, 1H), 1.78 (m, 1H), 1.46 (m, 2H), 1.23 (m, 1H), 1.03 (m, 1H). MS (ES) for C₁₆H₂₀N₇, found 310.1 (MH⁺).

### Example 11

### 3-(2-aminopyrimidin-4-yl)-N,N-dipropyl-1H-pyrrolo[2,3-b]pyridin-4-amine

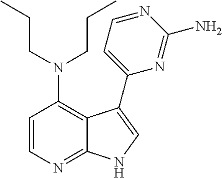

The same procedure in Example 4 was used to synthesize the title compound wherein N,N-dipropylamine was substituted for 4-methylpiperizine. ¹H-NMR (400 MHz, DMSO-d₆): δ 12.00 (s, 1H), 8.15 (d, 1H), 8.03 (d, 1H), 7.73 (s, 1H), 6.99 (d, 1H), 6.67 (d, 1H), 6.39 (s, 2H), 3.05 (t, 4H), 1.41 (m, 4H), 0.71 (m, 6H). MS (ES) for C₁₇H₂₃N₆, found 311.1 (MH+).

### Example 12

### 4-(4-pyrrolidin-1-yl-1H-pyrrolo[2,3-b]pyridin-3-yl)pyrimidin-2-amine

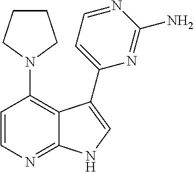

The same procedure in Example 4 was used to synthesize the title compound wherein pyrrolidine was substituted for 4-methylpiperizine. ¹H-NMR (400 MHz, DMSO-d₆): δ 8.15 (d, 1H), 7.95 (d, 1H), 7.56 (s, 1H), 6.75 (d, 1H), 6.44 (m, 3H), 3.09 (m, 4H), 1.77 (m, 4H). MS (ES) for C₁₅H₁₇N₆, found 281.1 (MH+).

### Example 13

### 4-[4-(3,5-dimethylpiperidin-1-yl)-1H-pyrrolo[2,3-b]pyridin-3-yl]pyrimidin-2-amine

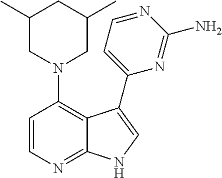

The same procedure in Example 4 was used to synthesize the title compound wherein 3,5-dimethylpiperidine was substituted for 4-methylpiperizine. ¹H-NMR (400 MHz, DMSO-d₆): δ 11.97 (s, 1H), 8.2 (d, 1H), 8.08 (d, 1H), 7.68 (s, 1H), 6.9 (d, 1H), 6.65 (d, 1H), 6.42 (s, 2H), 3.35 (m, 2H), 2.06 (t, 2H), 1.7 (m, 3H), 0.7 (d, 6H), 0.6 (q, 1H). MS (ES) for C₁₈H₂₃N₆, found 323.2 (MH+).

### Example 14

### N-methyl-4-(4-piperidin-1-yl-1H-pyrrolo[2,3-b]pyridin-3-yl)pyrimidin-2-amine

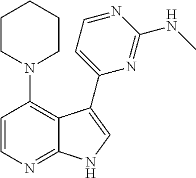

A similar procedure as in Example 4 was used to synthesize the title compound wherein piperidine was substituted for the amine. ¹H-NMR (400 MHz, DMSO-d₆): δ 12.04 (s, 1H), 8.27 (d, 1H), 8.08 (d, 1H), 7.79 (s, 1H), 7.05 (d, 1H), 6.85 (q, 1H), 6.68 (d, 1H), 2.95 (m, 4H), 2.86 (d, 3H), 1.51 (m, 6H). MS (ES) for C₁₇H₂₁N₆, found 309.0 (MH+).

### Example 15

### 4-(4-(4-methylpiperidin-14)-1H-pyrrolo[2,3-b]pyridine-3-yl)pyrimidin-2-amine

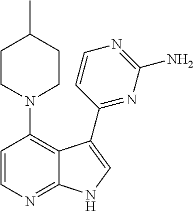

The same procedure in Example 4 was used to synthesize the title compound wherein 4-methylpiperidine was substituted for the amine. Purification by preparative HPLC gave 4-(4-(4-methylpiperidin-1-yl)-1H-pyrrolo[2,3-b]pyridine-3-yl)pyrimidin-2-amine (39 mg, 14% over three steps). ¹H-NMR (400 MHz, CD₃OD): δ 8.22 (d, 1H), 8.07 (d, 1H), 7.74 (s, 1H), 7.08 (d, 1H), 6.77 (d, 1H), 3.42-3.14 (m, 4H), 1.75-1.55 (m, 5H), 1.05 (m, 3H). MS (ES) for C₁₇H₂₀N₆ found 309.2 (MH+).

### Example 16

### 4-[4-(4-ethylpiperazin-1-yl)-1H-pyrrolo[2,3-b]pyridin-3-yl]pyrimidin-2-amine

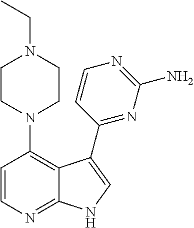

The same procedure in Example 4 was used to synthesize the title compound wherein 4-ethylpiperidine was substituted for the amine. Purification by preparative HPLC gave 4-[4-(4-ethylpiperazin-1-yl)-1H-pyrrolo[2,3-b]pyridin-3-yl]pyrimidin-2-amine (109 mg, 38% over three steps). ¹H-NMR (400 MHz, CD₃OD): δ 8.29 (d, 1H), 8.17 (d, 1H), 7.84 (s, 1H) 7.09 (d, 1H), 6.84 (d, 1H), 3.48-3.01 (m, 8H), 2.98 (q, 2H), 1.27 (t, 3H). MS (EI) for C₁₇H₂₁N₇ found 324.2 (MH+).

### Example 17

### 4-[4-(4-aminopiperidin-1-yl)-1H-pyrrolo[2,3-b]pyridin-3-yl]pyrimidin-2-amine

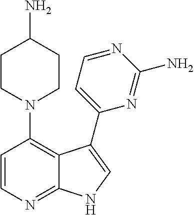

The same procedure in Example 4 was used to synthesize the title compound wherein 4-aminopiperidine was substituted for the amine. ¹H-NMR (400 MHz, DMSO-d₆): 8.26 (d, 1H), 8.08 (d, 1H), 7.76 (s, 1H), 7.04 (d, 1H), 6.7 (d, 1H), 6.4 (s, 2H), 3.34 (m, 1H), 2.7 (m, 2H), 2.62 (m, 2H), 1.7 (m, 2H), 1.46 (m, 2H). MS (ES) for C₁₆H₁₉N₇, found 310.2 (MH+).

### Example 18

### 4-[4-(3,5-dimethylpiperazin-1-yl)-1H-pyrrolo[2,3-b]pyridin-3-yl]pyrimidin-2-amine

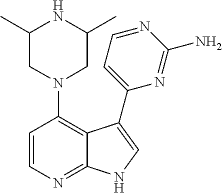

The same procedure in Example 4 was used to synthesize the title compound wherein 3,5 dimethyl-piperazine was substituted for the amine. ¹H-NMR (400 MHz, DMSO-d₆): 8.2 (d, 1H), 8.06 (d, 1H), 7.68 (s, 1H), 6.9 (d, 1H), 6.63 (d, 1H), 6.44 (s, 2H), 3.22 (d, 2H), 2.82 (m, 2H), 2.1 (m, 2H), 0.82 (d, 6H). MS (ES) for C₁₇H₂₂N₇, found 324.1 (MH+).

Using analogous synthetic techniques as in Scheme 2 and Example 4, and substituting with the appropriate alternative starting materials, the following compounds were prepared. Alternative starting materials were obtained commercially unless otherwise indicated.

N-(2-(3-(2-aminopyrimidin-4-yl)-1H-pyrrolo[2,3-b]pyridin-5-yl)ethyl)-2-(dimethylamino)acetamide. ¹H-NMR (400 MHz, DMSO-d₆): δ 12.1 (s, 1H), 8.72 (d, 1H), 8.29 (d, 1H), 8.13 (s, 1H), 8.12 (d, 1H), 7.79 (t, 1H), 7.03 (d, 1H), 6.50 (s, 2H), 3.45 (m, 2H), 2.90 (t, 2H), 2.81 (s, 2H), 2.11 (s, 6H). MS (ES) for C₁₇H₂₁N₇O, found 340.2 (MH+).

4-(4-((1S,4S)-5-methyl-2,5-diazabicyclo[2.2.1]heptan-2-yl)-1H-pyrrolo[2,3-b]pyridin-3-yl)pyrimidin-2-amine. ¹H-NMR (400 MHz, MeOH-d₄): δ 8.25 (d, 1H), 8.10 (d, 1H), 8.04 (s, 1H), 7.19 (d, 1H), 6.89 (d, 1H), 4.85 (m, 1H), 4.34 (s, 1H), 3.68-3.86 (m, 2H), 2.93 (s, 3H), 2.35-2.48 (m, 2H). MS (ES) for C₁₇H₁₉N₇, found 322.2 (MH+).

6-(4-(piperidin-1-yl)-1H-pyrrolo[2,3-b]pyridin-3-yl)pyrimidine-2,4-diamine. ¹H-NMR (400 MHz, MeOH-d₄): δ 8.18 (d, 1H), 7.90 (s, 1H), 7.03 (d, 1H), 6.28 (s, 1H), 3.25 (m, 4H), 1.66 (m, 6H). MS (ES) for C₁₆H₁₉N₇, found 310.2 (MH+).

4-[4-(3,4,5-trimethylpiperazin-1-yl)-1H-pyrrolo[2,3-b]pyridin-3-yl]pyrimidin-2-amine. ¹H-NMR (400 MHz, DMSO): δ 12.0 (s, 1H), 8.2 (d, 1H), 8.06 (d, 1H), 7.68 (s, 1H), 6.92 (d, 1H), 6.62 (d, 1H), 6.46 (s, 2H), 3.22 (d, 2H), 2.35 (t, 2H), 2.22 (m, 2H), 2.14 (s, 3H), 0.87 (d, 6H). MS (EI) for C₁₈H₂₄N₇ found 338 (MH+).

4-{4-[4-(dimethylamino)piperidin-1-yl]-1H-pyrrolo[2,3-b]pyridin-3-yl}pyrimidin-2-amine. ¹H-NMR (400 MHz, DMSO): δ 12.0 (s, 1H), 8.2 (d, 1H), 8.09 (d, 1H), 7.72 (s, 1H), 7.0 (d, 1H), 6.68 (d, 1H), 6.42 (s, 2H), 3.36 (m, 2H), 2.56 (t, 2H), 2.15 (s, 6H), 1.64 (m, 2H), 1.52-1.4 (m, 2H). MS (EI) for C₁₈H₂₃N₇ found 338.1 (MH+).

4-[4-(4-phenylpiperazin-1-yl)-1H-pyrrolo[2,3-b]pyridin-3-yl]pyrimidin-2-amine ¹H-NMR (400 MHz, DMSO): δ 12.5 (s, 1H), 8.22 (d, 1H), 8.16 (d, 1H), 7.8 (s, 1H), 7.2 (m, 2H), 7.05 (d, 1H), 6.95 (d, 2H), 6.8 (m, 2H), 6.5 (s, 2H), 3.18 (d, 4H), 3.1 (d, 4H). MS (EI) for C₂₁H₂₁N₇ found 372.1 (MH+).

4-{4-[3-(dimethylamino)pyrrolidin-1-yl]-1H-pyrrolo[2,3-b]pyridin-3-yl}pyrimidin-2-amine ¹H-NMR (400 MHz, DMSO): δ 8.26(d, 1H), 7.92 (d, 1H), 7.5 (s, 1H), 6.72 (d, 1H), 6.5 (s, 2H), 6.4 (d, 1H), 3.36-3.28 (m, 1H), 3.22-3.12 (m, 2H), 2.86 (t, 1H), 2.6 (q, 1H), 2.04 (s, 6H), 2.0-1.92 (m, 1H), 1.66-1.56 (m, 1H). MS (EI) for C₁₇H₂₂N₇ found 324.2 (MH+).

3-(2-aminopyrimidin-4-yl)-N-methyl-N-[2-(methyloxy)ethyl]-1H-pyrrolo[2,3-b]pyridin-4-amine. ¹H-NMR (400 MHz, DMSO-d₆): δ 8.15 (d, 1H), 8.04 (d, 1H), 7.72 (s, 1H), 6.98 (d, 1H), 6.65 (d, 1H), 6.39 (s, 2H), 3.38 (t, 2H), 3.23 (t, 2H), 3.13 (s, 3H), 2.75 (s, 3H). MS (ES) for C₁₅H₁₈N₆O, found 299.2 (MH+).

N-[3-(2-aminopyrimidin-4-yl)-1H-pyrrolo[2,3-b]pyridin-4-yl]-N,N′,N′-trimethylethane-1,2-diamine. ¹H-NMR (400 MHz, CD₃OD): δ 8.25 (d, 1H), 8.19 (s, 1H), 8.16 (d, 1H), 7.30 (d, 1H), 7.05 (d, 1H), 3.81 (t, 2H), 3.40-3.36 (m, 2H), 3.09 (s, 3H), 2.85 (s, 6H). MS (ES) for C₁₆H₂₁N₇ found 312.1 (MH+).

3-(2-aminopyrimidin-4-yl)-N-methyl-N-(1-methylpyrrolidin-3-yl)-1H-pyrrolo[2,3-b]pyridin-4-amine. ¹H-NMR (400 MHz, DMSO-d₆): δ 12.0 (s, 1H), 8.17 (d, 1H), 8.05 (d, 1H), 7.72 (d, 1H), 6.93 (d, 1H), 6.65 (d, 1H), 6.41 (s, 2H), 4.11 (m, 1H), 2.62 (s, 3H), 2.59 (m, 1H), 2.17 (s, 3H), 2.13 (m, 2H), 1.72 (m, 2H), 1.03 (m, 1H). MS (ES) for C₁₇H₂₁N₇, found 324.2 (MH+).

ethyl 4-[3-(2-aminopyrimidin-4-yl)-1H-pyrrolo[2,3-b]pyridin-4-yl]piperazine-1-carboxylate. ¹H-NMR (400 MHz, CD₃OD): δ 8.29 (d, 1H), 8.19 (s, 1H), 8.18 (d, 1H), 7.41 (d, 1H), 7.07 (d, 1H), 4.13 (q, 2H), 3.57 (m, 4H), 3.36 (m, 4H), 1.25 (t, 3H). MS (ES) for C₁₈H₂₁N₇O₂ found 368.2 (MH+).

4-[4-(3-aminopyrrolidin-1-yl)-1H-pyrrolo[2,3-b]pyridin-3-yl]pyrimidin-2-amine. ¹H-NMR (400 MHz, CD₃OD): δ 8.24 (d, 1H), 8.02 (d, 1H), 7.86 (s, 1H), 7.09 (d, 1H), 6.77 (d, 1H), 3.92 (m, 2H), 3.72 (m, 2H), 3.56 (m, 1H), 2.43 (m, 1H), 2.09 (m, 1H). MS (ES) for C₁₅H₁₇N₇, found 296.1 (MH+).

2-{1-[3-(2-aminopyrimidin-4-yl)-1H-pyrrolo[2,3-b]pyridin-4-yl]piperidin-3-yl}ethanol. ¹H-NMR (400 MHz, DMSO-d₆): δ 12.00 (s, 1H), 8.10 (d, 1H), 8.05 (d, 1H), 7.70 (s, 1H), 6.90 (d, 1H), 6.60 (d, 1H), 6.40 (br, 2H), 4.30 (br, 1H), 3.5 (m, 4H), 2.4 (m, 1H), 2.2 (m, 1H), 1.90 (s, 3H), 1.80 (m, 2H), 1.5 (m, 1H), 1.25 (m, 1H) and 1.00 (m, 1H). MS (ES) for C₁₈H₂₂N₆O, found 339.0 (MH+).

4-[4-(hexahydrocyclopenta[c]pyrrol-2(1H)-yl)-1H-pyrrolo[2,3-b]pyridin-3-yl]pyrimidin-2-amine. ¹H-NMR (400 MHz, DMSO): δ 12.8 (s, 1H), 8.4 (d, 1H), 8.08 (d, 1H), 7.82 (s, 1H), 7.4 (s, 2H), 7.0 (d, 1H), 6.7 (d, 1H), 3.6 (t, 2H), 3.05 (d, 1H), 2.62 (m, 2H), 2.5 (m, 2H), 1.74-1.5 (m, 3H), 1.42-1.1 (m, 2H). MS (EI) for C₁₈H₂₀N₆ found 321.1 (MH⁺).

4-[4-(5-methyl hexahydropyrrolo[3,4-c]pyrrol-2(1H)-yl)-1H-pyrrolo[2,3-b]pyridin-3-yl]pyrimidin-2-amine. ¹H-NMR (400 MHz, DMSO): δ 12.0 (s, 1H), 8.18 (d, 1H), 8.03 (d, 1H), 7.72 (s, 1H), 6.9 (d, 1H), 6.55 (d, 1H), 6.45 (s, 2H), 3.36 (t, 2H), 2.76-2.64 (m, 4H), 2.46 (d, 2H), 2.2 (s, 3H), 2.18 (t, 2H). MS (EI) for C₁₈H₂₂N₇ found 336.1 (MH⁺).

4-(4-(3-methoxypyrrolidin-1-yl)-1H-pyrrolo[2,3-b]pyridin-3-yl)pyrimidin-2-amine. ¹H-NMR (400 MHz, DMSO-d₆): δ 11.8 (s, 1H), 8.14 (d, 1H), 7.95 (d, 1H), 7.56 (s, 1H), 6.75 (d, 1H), 6.45 (s, 2H), 6.44 (d, 1H), 3.91 (m, 1H), 3.32 (m, 1H), 3.16 (m, 1H), 3.14 (s, 3H), 3.09 (m, 2H), 1.94 (m, 1H), 1.82 (m, 1H). MS (ES) for C₁₆H₁₈N₆O, found 311.2 (MH+).

4-{4-[(3R)-3-aminopyrrolidin-1-yl]-1H-pyrrolo[2,3-b]pyridin-3-yl}pyrimidin-2-amine. ¹H-NMR (400 MHz, CDCl₃ with CD₃OD): δ 8.18 (d, 1H), 8.0 (d, 1H), 7.56 (d, 1H), 6.92 (d, 1H), 6.51 (d, 1H), 3.7 (m, 1H), 3.5 (m, 1H), 3.4 (m, 1H), 3.23 (m, 1H), 2.98 (dd, 1H), 2.17 (m, 1H), 1.62 (m, 1H). MS (ES) for C₁₅H₁₇N₇, found 296.1 (MH+).

{1-[3-(2-aminopyrimidin-4-yl)-1H-pyrrolo[2,3-b]pyridin-4-yl]pyrrolidin-2-yl}methanol ¹H-NMR (400 MHz, CDCl₃): δ 8.13 (d, 1H), 7.97 (d, 1H), 7.64 (s, 1H), 6.84 (d, 1H), 6.65 (d, 1H), 6.36 (br s, 2H), 3.83 (m, 1H), 3.43 (m, 1H), 3.36 (m, 1H), 3.20 (m, 1H), 2.89 (m, 1H), 1.98 (m, 1H), 1.86 (s, 6H, di-acetic acid salt), 1.78 (m, 1H), 1.64 (m, 1H), 1.58 (m, 1H). MS (ES) for C₁₆H₁₈N₆O, found 311.1 (MH+).

N-{1-[3-(2-aminopyrimidin-4-yl)-1H-pyrrolo[2,3-b]pyridin-4-yl]pyrrolidin-3-yl}-N-methylacetamide. ¹H-NMR (400 MHz, DMSO-d₆): δ 8.29 (d, 1H), 8.05 (m, 1H), 7.75 (s, 1H), 6.91 (m, 1H), 6.65 (m, 1H), 3.25-3.62 (m, 3H), 2.80 (s, 1H), 2.54-2.70 (s, 2H), 1.88-2.09 (m, 7H) MS (ES) for C₁₈H₂₁N₇O, found 352.2 (MH+).

N-{1-[3-(2-aminopyrimidin-4-yl)-1H-pyrrolo[2,3-b]pyridin-4-yl]pyrrolidin-3-yl}acetamide. ¹H-NMR (400 MHz, MeOH-d₄): δ 8.20 (d, 1H), 7.98 (d, 1H), 7.63 (s, 1H), 6.94 (d, 1H), 6.59 (d, 1H), 4.26-4.33 (m, 1H), 3.46-3.54 (m, 1H), 3.34-3.37 (m, 1H), 3.18-3.27 (m, 1H), 3.09-3.15 (m, 1H), 2.10-2.19 (m, 1H), 1.97 (s, 3H), 1.90 (s, 3H), 1.76-1.85 (m, 1H). MS (ES) for C₁₇H₂₀N₇O, found 338.1 (MH⁺).

(3R)-1-[3-(2-aminopyrimidin-4-yl)-1H-pyrrolo[2,3-b]pyridin-4-yl]pyrrolidin-3-ol. ¹H-NMR (400 MHz, CDCl₃ with CD₃OD): δ 8.16 (d, 1H), 7.95 (d, 1H), 7.57 (s, 1H), 6.92 (d, 1H), 6.52 (d, 1H), 4.37 (m, 1H), 3.46 (m, 2H), 3.26 (m, 1H), 3.15 (dd, 1H), 2.05 (m, 1H), 2.03 (m, 1H). MS (ES) for C₁₅H₁₆N₆O, found 297.2 (MH+).

4-[4-(1-methylhexahydropyrrolo[3,4-b]pyrrol-5(1H)-yl)-1H-pyrrolo[2,3-b]pyridin-3-yl]pyrimidin-2-amine. ¹H-NMR (400 MHz, DMSO): δ 8.12 (d, 1H), 8.0 (d, 1H), 7.65 9s, 1H), 6.86 (d, 1H), 6.42 (d, 1H), 6.4 (s, 2H), 3.16-2.8 (m, 5H), 2.74-2.64 (m, 1H), 2.26-2.16 (m, 1H), 2.14 (s, 3H), 1.94-1.8 (m, 2H), 1.54-1.44 (m, 1H). MS (EI) for C₁₈H₂₂N₇ found 336.2 (MH+).

3-(2-aminopyrimidin-4-yl)-N-methyl-N-(1-methylpiperidin-4-yl)-1H-pyrrolo[2,3-b]pyridin-4-amine. ¹H-NMR (400 MHz, DMSO-d₆): δ 11.9 (s, 1H), 8.17 (d, 1H), 8.04 (d, 1H), 7.65 (s, 1H), 6.80 (d, 1H), 6.65 (d, 1H), 6.46 (s, 2H), 3.22 (m, 1H), 2.69 (s, 3H), 2.62 (m, 2H), 2.01 (s, 3H), 1.59 (m, 2H), 1.41 (m, 2H), 1.22 (m, 2H). MS (ES) for C₁₈H₂₃N₇, found 338.2 (MH+).

4-{4-[(3S)-3-aminopyrrolidin-1-yl]-1H-pyrrolo[2,3-b]pyridin-3-yl}pyrimidin-2-amine. ¹H-NMR (400 MHz, d-6DMSO): δ 8.15 (m, 1H), 7.87 (m, 1H), 7.52 (m, 1H), 6.77 (m, 1H), 6.39 (m, 3H), 4.21 (m, 2H), 3.39-3.05 (m, 4H), 2.77 (m, 1H), 1.88 (m, 1H), 1.43 (m, 1H). MS (ES) for C₁₅H₁₇N₇, found 296.0 (MH+).

4-{4-[3-(methylamino)pyrrolidin-1-yl]-1H-pyrrolo[2,3-b]pyridin-3-yl}pyrimidin-2-amine. ¹H-NMR (400 MHz, MeOH-d₄): δ 8.25 (d, 1H), 8.11 (s, 1H), 8.04 (d, 1H), 7.32 (d, 1H), 6.86 (d, 1H), 4.12 (m, 1H), 3.75-3.93 (m, 4H), 2.72 (s, 3H), 2.42-2.53 (m, 1H), 2.18-2.29 (m, 1H). MS (ES) for C₁₆H₁₉N₇, found 310.2 (MH⁺).

3-(2-aminopyrimidin-4-yl)-N-methyl-N-(1-methylethyl)-1H-pyrrolo[2,3-b]pyridin-4-amine. ¹H-NMR (400 MHz, MeOH-d₄): δ 8.20 (d, 1H), 8.05 (d, 1H), 7.71 (s, 1H), 6.98 (d, 1H), 6.76 (d, 1H), 3.82 (m, 1H), 2.75 (s, 3H), 1.95 (s, 3H), 0.93 (d, 6H). MS (ES) for C₁₅H₁₉N₆, found 283.2 (MH+).

4-(4-(2-(aminomethyl)pyrrolidin-1-yl)-1H-pyrrolo[2,3-b]pyridin-3-ylpyrimidin-2-amine. ¹H-NMR (400 MHz, DMSO-d₆): δ 11.8 (s, 1H), 8.14 (d, 1H), 7.96 (d, 1H), 7.61 (s, 1H), 6.79 (d, 1H), 6.63 (d, 1H), 6.38 (s, 2H), 3.77 (m, 1H), 3.15 (m, 1H), 2.89 (m, 1H), 2.66 (m, 2H), 1.98 (m, 1H), 1.78 (m, 1H), 1.65 (m, 1H), 1.54 (m, 1H). MS (ES) for C₁₆H₁₉N₇, found 310.2 (MH+).

(R)-4-(4-(2-(methoxymethyl)pyrrolidin-1-yl)-1H-pyrrolo[2,3-b]pyridin-3-yl)pyrimidin-2-amine. ¹H-NMR (400 MHz, DMSO-d₆): δ 11.9 (s, 1H), 8.14 (d, 1H), 7.98 (d, 1H), 7.63 (s, 1H), 6.80 (d, 1H), 6.65 (d, 1H), 6.40 (s, 2H), 3.93 (m, 1H), 3.30 (m, 1H), 3.24 (m, 2H), 3.19 (s, 3H), 2.93 (m, 1H), 1.98 (m, 1H), 1.72 (m, 2H), 1.62 (m, 1H). MS (ES) for C₁₇H₂₀N₆O, found 325.2 (MH+).

(3S)-1-[3-(2-aminopyrimidin-4-yl)-1H-pyrrolo[2,3-b]pyridin-4-yl]pyrrolidin-3-ol. ¹H-NMR (400 MHz, CDCl₃ with CD₃OD): δ 8.17 (d, 1H), 7.95 (d, 1H), 7.58 (s, 1H), 6.92 (d, 1H), 6.54 (d, 1H), 4.38 (m, 1H), 3.48 (m, 3H), 3.2 (d, 1H), 2.04 (m, 1H), 1.88 (m, 1H). MS (ES) for C₁₅H₁₆N₆O, found 297.2 (MH+).

{(2R)-1-[3-(2-aminopyrimidin-4-yl)-1H-pyrrolo[2,3-b]pyridin-4-yl]pyrrolidin-2-yl}methanol. ¹H-NMR (400 MHz, CDCl₃): δ 11.87 (br s, 1H), 8.13 (d, 1H), 7.97 (d, 1H), 7.64 (br d, 1H), 6.84 (d, 1H), 6.65 (d, 1H), 6.36 (br s, 2H), 4.74 (t, 1H), 3.83 (m, 1H), 3.40 (m, 2H), 3.20 (m, 1H), 2.89 (m, 1H), 1.98 (m, 1H), 1.79 (m, 1H), 1.69 (m, 1H), 1.58 (m, 1H). MS (ES) for C₁₆H₁₈N₆O, found 311.2 (MH+).

{(2S)-1-[3-(2-aminopyrimidin-4-O-1H-pyrrolo[2,3-b]pyridin-4-yl]pyrrolidin-2-yl}methanol. ¹H-NMR (400 MHz, DMSO-d₆): δ 11.90 (s, 1H), 8.10 (s, 1H), 8.0 (d, 1H), 7.60 (s, 1H), 6.80 (d, 1H), 6.60 (d, 1H), 6.40 (br, 2H), 4.80 (t, 1H), 3.80 (br, 1H), 3.42 (m, 1H), 3.40 (m, 1H), 3.20 (m, 1H), 2.85 (m, 1H), 2.0 (m, 1H), 1.80 (m, 1H), 1.65 (m, 1H) and 1.60 (m, 1H). MS (ES) for C₁₆H₁₈N₆O, found 311.0 (MH+).

2-{[3-(2-aminopyrimidin-4-yl)-1H-pyrrolo[2,3-b]pyridin-4-yl]amino}propane-1,3-diol. ¹H-NMR (400 MHz, DMSO-d₆): δ 10.20 (s, 1H), 8.10 (s, 1H), 8.05 (d, 1H), 7.05 (d, 1H), 6.35 (br, 2H), 6.25 (d, 1H), 5.25 (br, 2H), 3.70 (m, 2H), 3.60 (m, 2H), 3.25 (m, 1H) and 1.91.

4-(4-(hexahydropyrrolo[1,2-a]pyrazin-2(1H)-yl)-1H-pyrrolo[2,3-b]pyridin-3-yl)pyrimidin-2-amine. ¹H-NMR (400 MHz, DMSO-d₆): δ 12.1 (s, 1H), 8.20 (d, 1H), 8.09 (d, 1H), 7.72 (s, 1H), 6.95 (d, 1H), 6.69 (d, 1H), 6.42 (s, 1H), 4.06 (s, 2H), 3.47 (d, 1H), 3.30 (d, 1H), 2.95 (t, 1H), 2.82 (d, 1H), 2.67 (m, 1H), 2.42 (t, 1H), 2.00-2.20 (m, 3H), 1.91 (s, 3H), 1.54-1.70 (m, 3H), 1.17-1.29 (m, 1H). MS (EI) for C₁₈H₂₁N₇, found 336.2 (MH+).

1-[3-(2-aminopyrimidin-4-yl)-1H-pyrrolo[2,3-b]pyridin-4-yl]piperidin-4-ol. ¹H-NMR (400 MHz, DMSO-d₆): δ 11.98 (s, 1H), 8.19 (d, 1H), 8.05 (d, 1H), 7.70 (s, 1H), 6.99 (d, 1H), 6.67 (d, 1H), 6.39 (s, 1H), 4.68 (d, 1H), 3.54 (m, 1H), 3.23 (m, 2H), 2.68 (m, 2H), 1.66 (m, 2H), 1.42 (m, 2H). MS (EI) for C₁₇H₁₈N₆O found 311.2 (MH+).

4-(4-(hexahydropyrrolo[3,4-b]pyrrol-1(2H)-yl)-1H-pyrrolo[2,3-b]pyridin-3-yl)pyrimidin-2-amine. ¹H-NMR (400 MHz, DMSO-d₆): δ 8.2(d, 1H), 8.0 (d, 1H), 7.68 (s, 1H), 6.8 (d, 1H), 6.55 (d, 1H), 6.43 (s, 2H), 4.07 (t, 1H), 3.3 (m, 1H), 2.9 (q, 1H), 2.8-2.65 (m, 5H), 1.85 (m, 1H), 1.48 (m, 1H). MS (EI) for C₁₇H₂₀N₇ found 322.1 (MH+).

4-[4-(4-methyl-1,4-diazepan-1-yl)-1H-pyrrolo[2,3-b]pyridin-3-yl]pyrimidin-2-amine. ¹H-NMR (400 MHz, CD₃OD): δ 8.24 (d, 1H), 8.12 (d, 1H), 8.12 (s, 1H), 7.25 (d, 1H), 7.02 (d, 1H), 4.10-3.97 (m, 2H), 3.96-3.84 (m, 2H), 3.53-3.46 (m, 2H), 2.90 (s, 3H), 2.29-2.20 (m, 2H). MS (EI) for C₁₇H₂₁N₇ found 324.2 (MH+).

4-[4-(3,4-dimethylpiperazin-1-yl)-1H-pyrrolo[2,3-b]pyridin-3-yl]pyrimidin-2-amine. ¹H-NMR (400 MHz, DMSO-d₆): δ 12.0 (s, 1H), 8.21 (d, 1H), 8.07 (d, 1H), 7.70 (d, 1H), 6.93 (d, 1H), 6.64 (d, 1H), 6.43 (s, 2H), 3.21 (m, 2H), 2.68 (m, 2H), 2.30 (m, 2H), 2.15 (s, 3H), 2.10 (m, 1H), 0.82 (d, 3H). MS (EI) for C₁₇H₂₁N₇, found 324.2 (MH+).

N′-[3-(2-aminopyrimidin-4-yl)-1H-pyrrolo[2,3-b]pyridin-4-yl]-N,N-dimethylethane-1,2-diamine. ¹H-NMR (400 MHz, DMSO-d₆): δ 11.83 (s, 1H), 10.10 (m, 1H), 8.08 (s, 1H), 8.03 (d, 1H), 7.81 (d, 1H), 7.05 (d, 2H), 6.15 (d, 1H), 3.26 (m, 2H), 2.63 (m, 2H), 2.19 (s, 6H). MS (EI) for C₁₅H₁₉N₇, found 298.1 (MH+).

(S)-4-(4-(3-fluoropyrrolidin-1-yl)-1H-pyrrolo[2,3-b]pyridin-3-yl)pyrimidin-2-amine. ¹H-NMR (400 MHz, DMSO-d₆): δ 11.9 (s, 1H), 8.13 (d, 1H), 7.98 (d, 1H), 7.59 (s, 1H), 6.77 (d, 1H), 6.49 (d, 1H), 6.46 (s, 2H), 5.29 (m, 1H), 3.42 (m, 1H), 3.31 (m, 2H), 3.14 (m, 1H), 2.07 (m, 2H). MS (EI) for C₁₅H₁₅FN₆, found 299.2 (MH+).

(S)-4-(4-(2-(methoxymethyl)pyrrolidin-1-yl)-1H-pyrrolo[2,3-b]pyridin-3-yl)pyrimidin-2-amine. ¹H-NMR (400 MHz, DMSO-d₆): δ 11.9 (s, 1H), 8.14 (d, 1H), 7.98 (d, 1H), 7.63 (s, 1H), 6.80 (d, 1H), 6.65 (d, 1H), 6.40 (s, 2H), 3.93 (m, 1H), 3.30 (m, 1H), 3.24 (m, 2H), 3.19 (s, 3H), 2.93 (m, 1H), 1.98 (m, 1H), 1.72 (m, 2H), 1.62 (m, 1H). MS (EI) for C₁₇H₂₀N₆O, found 325.2 (MH+).

4-(4-(3,3-dimethylpyrrolidin-1-yl)-1H-pyrrolo[2,3-b]pyridin-3-yl)pyrimidin-2-amine. ¹H-NMR (400 MHz, DMSO-d₆): δ 11.8 (s, 1H), 8.13 (d, 1H), 7.90 (d, 1H), 7.47 (d, 1H), 6.71 (d, 1H), 6.46 (s, 2H), 6.37 (d, 1H), 3.17 (t, 2H), 2.94 (s, 2H), 1.51 (t, 2H), 1.02 (s, 6H). MS (ES) for C₁₇H₂₀N₆, found 309.2 (MH+).

### Representative Conditions under Scheme 2B

### Example 19

### 3-(2-aminopyrimidin-4-O—N-methyl-N-phenethyl-1H-pyrrolo[2,3-b]pyridin-4-amine

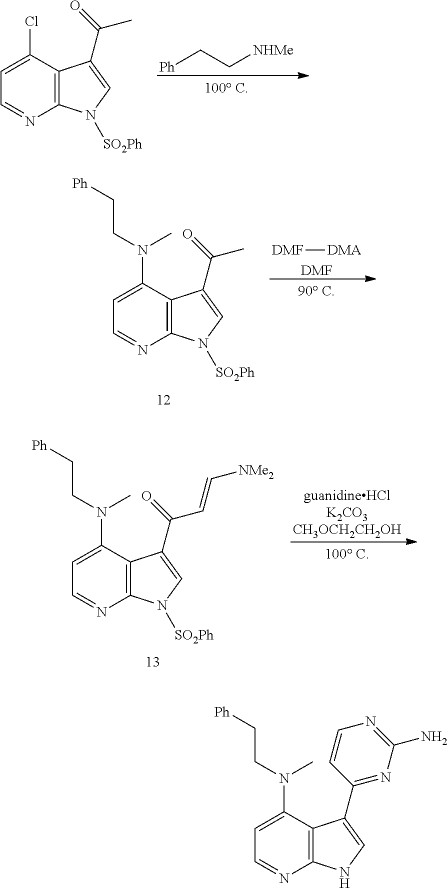

### 1-(4-(methyl(phenethyl)amino)-1-(phenylsulfonyl)-1H-pyrrolo[2,3-b]pyridin-3-yl)ethanone

A solution of 1-(4-chloro-1-(phenylsulfonyl)-1H-pyrrolo[2,3-b]pyridin-3-yl)ethanone (200 mg, 0.599 mmol) in N-methylphenethylamine (5 mL) was stirred at 100° C. for 2 hours. The resulting mixture was then diluted with water and extracted three times with ethyl acetate. The combined organic extracts were washed with brine and 5% aqueous HCl solution, dried over sodium sulfate, filtered and concentrated. The crude product was purified by flash chromatography to give the title compound 12 (85 mg, 33%). MS (ES) for C₂₄H₂₃N₃O₃S, found 434.2 (MH+).

### (E)-3-(dimethylamino)-1-(4-(methyl(phenethyl)amino)-1-(phenylsulfonyl)-1H-pyrrolo[2,3-b]pyridin-3-yl)prop-2-en-1-one

The title compound was synthesized in a manner similar to Example 1, wherein 1-(4-(dimethylamino)-1-((2-(trimethylsilyl)ethoxy)methyl)-1H-pyrazolo[3,4-d]pyrimidin-3-yl)ethanone was substituted with 1-(4-(methyl(phenethyl)amino)-1-(phenylsulfonyl)-1H-pyrrolo[2,3-b]pyridin-3-yl)ethanone 13. The crude product was carried onto the next step without any purification. MS (ES) for C₂₇H₂₈N₄O₃S, found 489.2 (MH+).

### 3-(2-aminopyrimidin-4-yl)-N-methyl-N-phenethyl-1H-pyrrolo[2,3-b]pyridin-4-amine

A mixture of (E)-3-(dimethylamino)-1-(4-(methyl(phenethyl)amino)-1-(phenylsulfonyl)-1H-pyrrolo[2,3-b]pyridin-3-yl)prop-2-en-1-one (96 mg, 0.19 mmol), guanidine hydrogen chloride (100 mg, 1.05 mmol) and potassium carbonate (145 mg, 1.05 mmol) in 2-methoxyethanol (10 mL) was stirred at 100° C. for 18 hours. The resulting mixture was then evaporated to dryness. The crude product was then purified by preparative HPLC to afford the title compound (7.8 mg, 11%). ¹H-NMR (400 MHz, DMSO-d₆): δ 8.05 (d, 1H), 7.95 (d, 1H), 7.69 (s, 1H), 7.23-7.10 (m, 3H), 7.05-7.00 (m, 2H), 6.69 (d, 1H), 6.65 (d, 1H), 6.35 (s, 2H), 3.27 (t, 2H), 2.73 (s, 3H), 2.70 (t, 2H). MS (ES) for C₂₀H₂₀N₆, found 345.2 (MH+).

### Representative Conditions under Scheme 3

### Example 20

### 3-(4-chloro-1H-pyrrolo[2,3-b]pyridin-3-yl)aniline

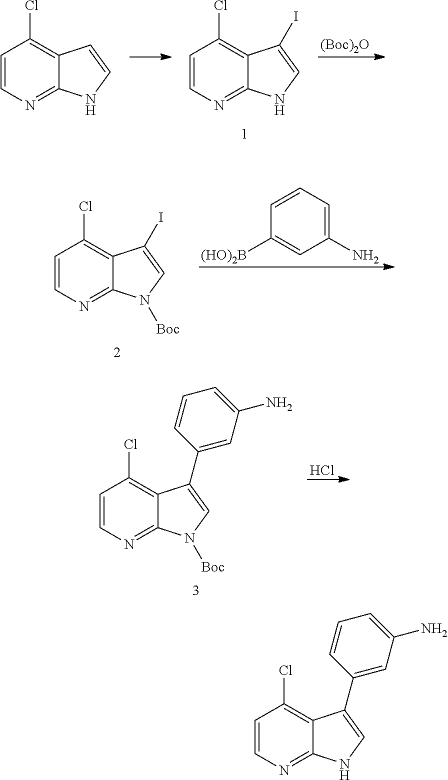

### 4-chloro-3-iodo-1H-pyrrolo[2,3-b]pyridine

Intermediate 1 was made following the known procedure as referenced in:

Lefoix, Myriam; Daillant, Jean-Philippe; Routier, Sylvain; Merour, Jean-Yves; Gillaizeau, Isabelle; Coudert, Gerard. Versatile and convenient methods for the synthesis of C-2 and C-3 functionalized 5-azaindoles. Synthesis (2005), (20), 3581-3588.

### tert-butyl 4-chloro-3-iodo-1H-pyrrolo[2,3-b]pyridine-1-carboxylate

A mixture of 4-chloro-3-iodo-1H-pyrrolo[2,3-b]pyridine 1 (1 g, 3.6 mmol), di-tert-butyl dicarbonate (0.807 g, 3.7 mmol), TEA (560 ul, 4 mmol), DMAP (catalytic amt. of crystals) and THF (20 mL) were stirred at room temperature overnight. The mixture was concentrated and then purified by silica gel chromatography to afford tert-butyl 4-chloro-3-iodo-1H-pyrrolo[2,3-b]pyridine-1-carboxylate 2 (1.33 g, 98%). ¹H-NMR (400 MHz, DMSO-d₆): δ 8.38-8.37 (d, 1H), 8.11 (s, 1H), 7.46-7.45 (d, 1H), 1.61 (s, 9H). MS (EI) for C₁₂H₁₂ClN₂O₂, found 379 (MH+).

### tert-butyl 3-(3-aminophenyl)-4-chloro-1H-pyrrolo[2,3-b]pyridine-1-carboxylate

Reaction conditions similar those described in Example 22 were employed to intermediate 2 to give tert-butyl 3-(3-aminophenyl)-4-chloro-1H-pyrrolo[2,3-b]pyridine-1-carboxylate 3. ¹H-NMR (400 MHz, CDCl₃): δ 8.43-8.42 (d, 1H), 7.61 (s, 1H), 7.22-7.18 (m, 2H), 6.90-6.88 (d, 1H), 6.82-6.81 (m, 1H), 6.74-6.72 (d, 1H), 3.75 (Br, s, 2H), 1.67 (s, 9H). MS (EI) for C₁₈H₁₈ClN₃O₂, found 344 (MH+).

### 3-(4-chloro-1H-pyrrolo[2,3-b]pyridin-3-yl)aniline

A solution of tert-butyl 3-(3-aminophenyl)-4-chloro-1H-pyrrolo[2,3-b]pyridine-1-carboxylate 3 (30 mg, 0.08 mmol) in 4M HCl in dioxane (5 mL) and DCM (5 mL) was stirred over 48 hours. The mixture was diluted with saturated NaHCO₃ and extracted 3 times with ethyl acetate. The organic fractions were combined, dried with sodium sulfate, filtered and concentrated under reduced pressure. The crude product was purified using silica chromatography to give 3-(4-chloro-1H-pyrrolo[2,3-b]pyridin-3-yl)aniline (4 mg, 19%). ¹H-NMR (400 MHz, CDCl₃): δ 10.54 (Br, s, 1H), 8.23-8.22 (d, 1H), 7.37 (s, 1H), 7.22-7.18 (t, 1H), 7.14-7.13 (d, 1H), 6.95-6.96 (d, 1H), 6.87 (s, 1H), 6.72-6.70 (d, 1H), 3.73 (br s, 2H). MS (EI) for C₁₃H₁₀ClN₃, found 244 (MH+).

Using analogous synthetic techniques as in Scheme 3 and Example 20, and substituting with the appropriate alternative starting materials, the following compounds were prepared. Alternative starting materials were obtained commercially unless otherwise indicated.

3-(4-(piperidin-1-yl)-1H-pyrrolo[2,3-b]pyridin-3-yObenzamide. ¹H-NMR (400 MHz, CD₃OD): δ 8.12 (t, 1H), 8.04 (d, 1H), 7.76 (q, 2H), 7.49 (t, 1H), 7.33 (s, 1H), 6.70 (d, 1H), 2.93 (br, 4H), 1.35 (br, 2H), 1.29 (br, 4H). MS (ES) for C₁₉H₂₀N₄O, found 321.2 (MH+).

4-(methyloxy)-6-(4-piperidin-1-yl-1H-pyrrolo[2,3-b]pyridin-3-yl)pyrimidin-2-amine. ¹H-NMR (400 MHz, MeOH-d₄): δ 8.06 (d, 1H), 7.66 (s, 1H), 6.74 (d, 1H), 6.52 (s, 1H), 3.94 (s, 3H), 3.04 (m, 4H), 1.56 (m, 6H). MS (ES) for C₁₇H₂₀N₆O, found 325.2 (MH+).

### Representative Conditions under Scheme 4

### Example 21

### 3-(2-Aminopyridin-4-yl)-N,N-dimethyl-1H-pyrazolo[3,4-b]pyridin-4-amine

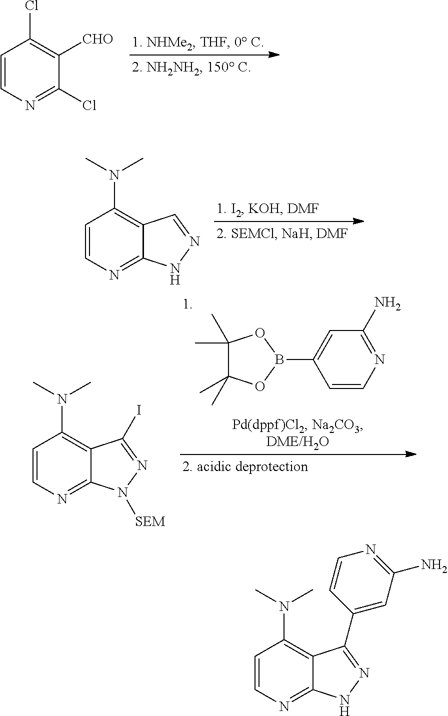

### 2,4-dichloronicotinaldehyde

To a 0° C. solution of 2,4-dichloronicotinaldehyde (1.93 g, 11 mmol, prepared by a literature procedure: J. Org. Chem. 1991, 4793.) in THF (20 mL) was added NHMe₂ (11 mL, 22 mmol, 2 M in THF). The resulting mixture was stirred for 30 min. The reaction mixture was diluted with EtOAc, washed with H₂O, and dried over Na₂SO₄. Removal of the solvents under reduced pressure gave 1.2 g (59%) of the crude product which was used in the next step without further purification.

### N,N-dimethyl-1H-pyrazolo[3,4-b]pyridin-4-amine

The crude aldehyde (1.2 g, 6.48 mmol) obtained above was treated with excess hydrazine hydrate (10 mL) at 150° C. for 30 min. The mixture was cooled to ambient temperature. Hydrazine was removed under reduced pressure. The residue was stirred in water. Filtration of the mixture gave 0.6 g (57%) of N,N-dimethyl-1H-pyrazolo[3,4-b]pyridin-4-amine as a solid. ¹H-NMR (400 MHz, CDCl₃): δ 8.14 (s, 1H), 7.95 (d, 1H), 6.07 (d, 1H), 3.20 (s, 6H). MS (EI) for C₈H₁₀N₄, found 163.2 (MH+).

### 3-iodo-N,N-dimethyl-1H-pyrazolo[3,4-b]pyridin-4-amine

To a solution of N,N-dimethyl-1H-pyrazolo[3,4-b]pyridin-4-amine (0.6 g, 3.7 mmol) in DMF (15 mL) were added KOH (725 mg, 13 mmol) and I₂ (1.4 g, 5.5 mmol). The resulting mixture was stirred for 12 h. The mixture was diluted with EtOAc. The organic layer was washed with 10% aqueous Na₂SO₃ solution, 5% aqueous LiCl solution, and dried over Na₂SO₄. Removal of the solvents gave crude 3-iodo-N,N-dimethyl-1H-pyrazolo[3,4-b]pyridin-4-amine (1.0 g, 94%).

### 3-iodo-N,N-dimethyl-14(2-(trimethylsilyl)ethoxy)methyl)-1H-pyrazolo[3,4-b]pyridin-4-amine

To a solution of the crude 3-iodo-N,N-dimethyl-1H-pyrazolo[3,4-b]pyridin-4-amine (1.0 g, 3.5 mmol) in DMF (15 mL) was added NaH (220 mg, 5.5 mmol, 60% in mineral oil). The resulting mixture was stirred for 30 min, and then SEMCI (700 mg, 4.2 mmol) was added. The stirring was continued for 2 h. The reaction mixture was quenched with water, extracted with EtOAc, and dried over Na₂SO₄. Removal of the solvents gave crude product which was purified by flash column chromatography to give 3-iodo-N,N-dimethyl-14(2-(trimethylsilyl)ethoxy)methyl)-1H-pyrazolo[3,4-b]pyridin-4-amine (630 mg, 40% over two steps). ¹H-NMR (400 MHz, DMSO-d₆): δ 8.34 (d, 1H), 6.68 (d, 1H), 5.75 (s, 2H), 3.67 (t, 2H), 3.14 (s, 6H), 0.91 (t, 2H), 0.11 (s, 9H). MS (EI) for C₁₄H₂₃IN₄OSi, found 419.2 (MH+).

### 3-(2-Aminopyridin-4-yl)-N,N-dimethyl-1H-pyrazolo[3,4-b]pyridin-4-amine

The Suzuki coupling of 3-iodo-N,N-dimethyl-14(2-(trimethylsilyl)ethoxy)methyl)-1H-pyrazolo[3,4-b]pyridin-4-amine with 2-aminopyridine-4-boronic acid pinacol ester and the SEM group deprotection were carried out similarly according to the procedure described in Example 22 to obtain 3-(2-Aminopyridin-4-yl)-N,N-dimethyl-1H-pyrazolo[3,4-b]pyridin-4-amine. ¹H-NMR (400 MHz, DMSO-d6): δ 13.61 (br s, 1H), 8.20 (d, 1H), 7.97 (d, 1H), 6.81 (m, 2H) 6.51 (d, 1H), 6.01 (br s, 2H), 2.72 (s, 6H). MS (EI) for C₁₃H₁₄N₆, found 255.1 (MH+).

### Representative Conditions under Scheme 5

### Example 22

### 4-[4-Methoxy-1H-pyrazolo[3,4-d]pyrimidin-3-yl]pyridin-2-amine

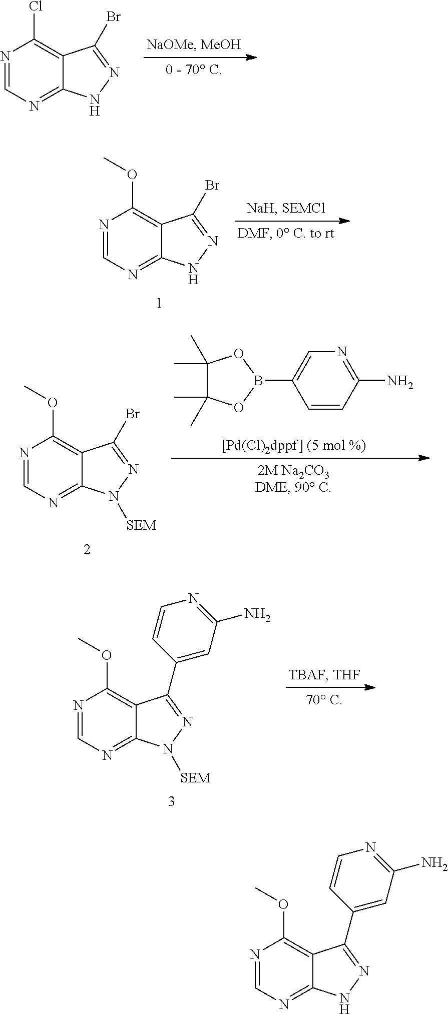

### 3-bromo-4-methoxy-1H-pyrazolo[3,4-d]pyrimidine

3-Bromo-4-chloro-1H-pyrazolo[3,4-c]pyrimidine (509 mg, 2.18 mmol) was treated with sodium methoxide (1.20 g, 22.1 mmol) in anhydrous methanol (30 mL) at 0° C., then at 70° C. for 2 h. After cooling to ambient temperature, the mixture was poured onto a 1M solution of sodium hydrogensulfate (30 mL) and extracted with ethyl acetate (2×80 mL). The combined organic layers were washed with water (60 mL) and brine (40 mL), and dried over magnesium sulfate. After purification by flash chromatography (75:25 hexanes/ethyl acetate), 3-bromo-4-methoxy-1H-pyrazolo[3,4-c]pyrimidine 1 was obtained as a white solid (325 mg, 65% yield). ¹H-NMR (400 MHz, DMSO-d₆): δ 8.59 (s, 1H), 4.12 (s, 3H). MS (EI) for C₆H₅BrN₄O, found 230 (MH+).

### 3-bromo-4-methoxy-1-[[2-(trimethylsilyl)ethoxy]methyl]-1H-pyrazolo[3,4-d]pyrimidine

A two-neck 100 mL round bottom flask was charged with sodium hydride (60% dispersion in oil; 82 mg, 2.05 mmol) and anhydrous dimethylformamide (5 mL) under nitrogen. A solution of 3-bromo-4-methoxy-1H-pyrazolo[3,4-d]pyrimidine 1 (319 mg, 1.39 mmol) in anhydrous dimethylformamide (2 mL) under nitrogen at 0° C. After stirring for 1 h, trimethylsilylethoxymethylchloride (90% tech; 0.28 mL, 1.42 mmol) was added dropwise via syringe. The reaction mixture was allowed to gradually warm to room temperature and stirred for 16 h. The mixture was poured into water (60 mL) and extracted with ethyl acetate (2×80 mL). The combined organic layers were washed with water (60 mL) and brine (40 mL), and dried over magnesium sulfate. After purification by flash chromatography (85:15 hexanes/ethyl acetate), 3-bromo-4-methoxy-1-[[2-(trimethylsilyl)ethoxy]methyl]-1H-pyrazolo[3,4-d]pyrimidine 2 was obtained as a white solid (403 mg, 81% yield). ¹H-NMR (400 MHz, CDCl₃): δ 8.60 (s, 1H), 5.77 (s, 2H), 4.23 (s, 3H), 3.69 (dd, 2H), 0.97 (dd, 2H), 0.00 (s, 9H). ¹³C-NMR (100 MHz, CDCl₃): δ 165.2, 157.9, 121.0, 104.6, 76.8, 68.7, 56.0, 19.1, 0.00. MS (EI) for C₁₂H₁₉BrN₄O₂Si, found 360 (MH+).

### 4-[4-Methoxy-1-[[2-(trimethylsilyl)ethoxy]methyl]-1H-pyrazolo[3,4-d]pyrimidin-3-yl]pyridin-2-amine

A pressure tube was charged with 3-bromo-4-methoxy-1-[[2-(trimethylsilyl)ethoxy]methyl]-1H-pyrazolo[3,4-d]pyrimidine 2 (79 mg, 0.220 mmol), 5-(4,4,5,5-tetramethyl-1,3,2-dioxaborolan-2-yl)pyridin-2-amine (79 mg, 0.59 mmol), dichloro[1,1′-bis(diphenylphosphino)ferrocene]palladium (II) dichloromethane adduct (9 mg, 0.011 mmol), a 2M solution of sodium carbonate (0.35 mL) and dimethoxyethane (3 mL). The mixture was bubbled with nitrogen for 10 minutes. The vessel was sealed and heated at 100° C. for 16 h. After cooling to room temperature, the mixture was poured into water (60 mL) and extracted with ethyl acetate (2×80 mL). The combined organic layers were washed with water (60 mL) and brine (40 mL), and dried over magnesium sulfate. Purification by flash chromatography (91:8:1 dichloromethane/methanol/28% (w/w) ammonium hydroxide) afforded 4-[4-Methoxy-1-[[2-(trimethylsilyl)ethoxy]methyl]-1H-pyrazolo[3,4-c]pyrimidin-3-yl]pyridin-2-amine 3 as a solid (76 mg, 93% yield). ¹H-NMR (400 MHz, CDCl₃): δ 8.67 (s, 1H), 8.22 (d, 1H), 7.45 (d, 1H), 7.31 (s, 1H), 5.88 (s, 2H), 4.67 (br s, 2H), 4.25 (s, 3H), 3.73 (dd, 2H), 1.00 (dd, 2H), 0.00 (s, 9H). ¹³C-NMR (100 MHz, CDCl₃): δ 165.3, 161.5, 158.4, 157.1, 151.1, 144.6, 142.6, 135.6, 115.1, 109.1, 76.8, 68.7, 56.1, 19.1, 0.0. MS (EI) for C₁₇H₂₄N₆O₂Si, found 373 (MH⁺).

### 4-[4-Methoxy-1H-pyrazolo[3,4-d]pyrimidin-3-yl]pyridin-2-amine

4-[4-Methoxy-1-[[2-(trimethylsilyl)ethoxy]methyl]-1H-pyrazolo[3,4-4 pyrimidin-3-yl]pyridin-2-amine (3, 76 mg, 0.204 mmol) in anhydrous THF (5 mL) was treated with a 1M solution of TBAF in tetrahydrofuran (1 mL, 1 mmol) at 70° C. for 7 h. After cooling to room temperature, the mixture was concentrated and loaded onto a column of silica gel and eluted with 91:8:1 dichloromethane/methanol/28% (w/w) ammonium hydroxide. The isolated product was further purified by preparative HPLC (reverse-phase, 0.1% TFA in acetonitrile/0.05% TFA in water). The combined fractions containing the target compound were stirred with basic ion exchange Bio-Rad® AG 1-x8 resin (200 mg, hydroxide form) for 10 min, until a basic pH was obtained, then filtered through fritted septum. After removal of volatiles in vacuo and freeze-drying, 4-[4-Methoxy-1H-pyrazolo[3,4-d]pyrimidin-3-yl]pyridin-2-amine was obtained as a yellowish solid (6% yield). ¹H-NMR (400 MHz, DMSO-d₆): δ 8.68 (s, 1H), 8.22 (br s, 2H), 8.02 (d, 1H), 7.83 (s, 1H), 7.54 (m, 1H), 4.20 (s, 3H). MS (EI) for C₁₁H₁₀N₆O, found 243 (MH+).

The following compounds were made in a manner analogous to Example 22:

### Example 23

### 4-(4-(dimethylamino)-1H-pyrazolo[3,4-b]pyridin-3-yl)pyridin-2(1H)-one

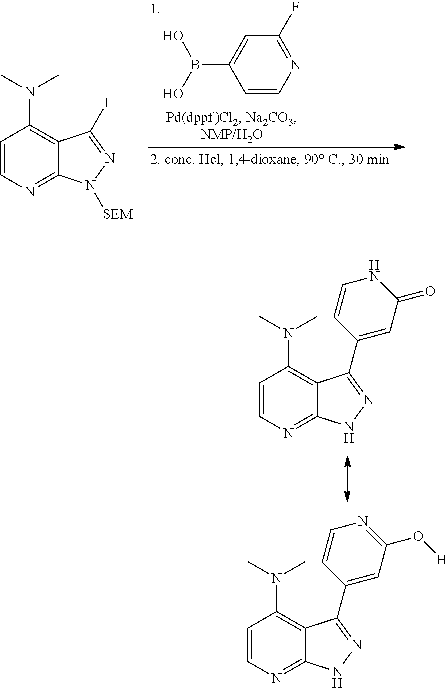

The Suzuki coupling of 3-iodo-N,N-dimethyl-14(2-(trimethylsilyl)ethoxy)methyl)-1H-pyrazolo[3,4-b]pyridin-4-amine with 2-fluoropyridine-4-boronic acid was carried out according to the procedure described in Example 22. The coupled product was dissolved in 1,4-dioxane (6 mL) and conc. HCl (2 mL). The resulting mixture was stirred for 30 min at 90° C., cooled to rt, and neutralized by slow addition of K₂CO₃. Filtration, followed by washes with water and MeOH gave 4-(4-(dimethylamino)-1H-pyrazolo[3,4-b]pyridin-3-yl)pyridin-2(1H)-one (42 mg, 27% over two steps). 4-(4-(dimethylamino)-1H-pyrazolo[3,4-b]pyridin-3-yl)pyridin-2(1H)-one tautomerizes into 4-(4-(dimethylamino)-1H-pyrazolo[3,4-b]pyridin-3-yl)pyridin-2-ol. ¹H-NMR (400 MHz, DMSO-d6): δ 8.19 (d, 1H), 7.42 (d, 1H), 6.65 (m, 2H), 6.52 (d, 1H), 2.76 (s, 6H). MS (EI) for C₁₃H₁₃N₅O, found 256.1 (MH+).

### Example 24

### 3-(2-Aminopyridin-4-yl)-N,N-dimethyl-1H-pyrazolo[3,4-d]pyrimidin-4-amine

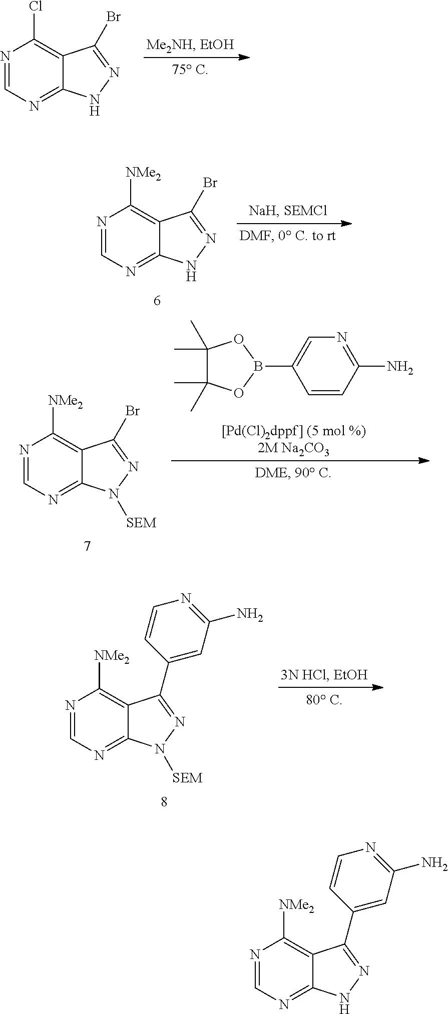

### 3-bromo-N,N-dimethyl-1H-pyrazolo[3,4-d]pyrimidin-4-amine

3-Bromo-4-chloro-1H-pyrazolo[3,4-d]pyrimidine (568 mg, 2.43 mmol) was treated with a 1M solution of dimethylamine in THF (2.5 mL, 2.5 mmol) in ethanol (20 mL) at 75° C. for 16 h. After cooling to RT, the mixture was concentrated under reduced pressure and azeotroped with acetonitrile. After purification by flash chromatography (91:8:1 dichloromethane/methanol/28% (w/w) ammonium hydroxide), 3-bromo-N,N-dimethyl-1H-pyrazolo[3,4-d]pyrimidin-4-amine 6 was obtained as a white solid (395 mg, 67% yield). ¹H-NMR (400 MHz, DMSO-d6): δ 8.25 (s, 1H), 3.36 (s, 6H). MS (EI) for C₇H₈BrN₅, found 243 (MH+).

### 3-bromo-N,N-dimethyl-1-[[2-(trimethylsilyl)ethoxy]methyl]-1H-pyrazolo[3,4-d]pyrimidin-4-amine

The title compound was synthesized in a manner similar to Example 22, wherein 3-bromo-4-methoxy-1H-pyrazolo[3,4-d]pyrimidine was substituted with 3-bromo-N,N-dimethyl-1H-pyrazolo[3,4-d]pyrimidin-4-amine. After purification by flash chromatography (85:15 hexanes/ethyl acetate), 3-bromo-N,N-dimethyl-1-[[2-(trimethylsilyl)ethoxy]methyl]-1H-pyrazolo[3,4-d]pyrimidin-4-amine 7 was obtained as a white solid (537 mg, quantitative yield). ¹H-NMR (400 MHz, CDCl₃): δ 8.38 (s, 1H), 5.72 (s, 2H), 4.23 (s, 3H), 3.68 (dd, 2H), 3.44 (s, 6H), 0.99 (dd, 2H), 0.00 (s, 9H). MS (EI) for C₁₃H₂₂BrN₅OSi, found 373 (MH⁺).

### 3-(2-aminopyridin-4-yl)-N,N-dimethyl-1-[[2-(trimethylsilyl)ethoxy]methyl]-1H-pyrazolo[3,4-d]pyrimidin-4-amine

The title compound was synthesized in a manner similar to Example 22, wherein 3-bromo-4-methoxy-1-[[2-(trimethylsilyl)ethoxy]methyl]-1H-pyrazolo[3,4-d]pyrimidine was substituted with 3-bromo-N,N-dimethyl-1-[[2-(trimethylsilyl)ethoxy]methyl]-1H-pyrazolo[3,4-d]pyrimidin-4-amine 7. After purification by flash chromatography (92:7:1 dichloromethane/methanol/28% (w/w) ammonium hydroxide), 3-(2-aminopyridin-4-yl)-N,N-dimethyl-1-[[2-(trimethylsilyl)ethoxy]methyl]-1H-pyrazolo[3,4-d]pyrimidin-4-amine 8 was obtained as a solid (83 mg, 70% yield). ¹H-NMR (400 MHz, CDCl₃): δ 8.48 (s, 1H), 8.19 (d, 1H), 6.92 (d, 1H), 6.87 (s, 1H), 5.82 (s, 2H), 4.75 (br s, 2H), 3.76 (dd, 2H), 3.04 (s, 6H), 1.00 (dd, 2H), 0.00 (s, 9H). ¹³C-NMR (100 MHz, CDCl₃): δ 161.6, 160.3, 158.0, 156.4, 149.9, 145.4, 115.8, 109.2, 100.7, 76.5, 68.5, 42.4, 19.1, 0.00. MS (EI) for C₁₈H₂₇N₇OSi, found 386 (MH+).

### 3-(2-aminopyridin-4-yl)-N,N-dimethyl-1H-pyrazolo[3,4-d]pyrimidin-4-amine

The title compound was synthesized in a manner similar to the coupling in Example 22, wherein 3-(1H-indazol-6-yl)-4-methoxy-1-[[2-(trimethylsilyl)ethoxy]methyl]-1H-pyrazolo[3,4-d]pyrimidine was substituted with 3-(2-aminopyridin-4-yl)-N,N-dimethyl-1-[[2-(trimethylsilyl)ethoxy]methyl]-1H-pyrazolo[3,4-d]pyrimidin-4-amine 8. After purification by flash chromatography (91:8:1 dichloromethane/methanol/28% (w/w) ammonium hydroxide), 3-(2-aminopyridin-4-yl)-N,N-dimethyl-1H-pyrazolo[3,4-d]pyrimidin-4-amine was obtained as a solid (10 mg, 18% yield). ¹H-NMR (400 MHz, CDCl₃): δ 8.50 (s, 1H), 8.20 (d, 1H), 8.02 (d, 1H), 6.92 (m, 1H), 6.6 (s, 1H), 4.73 (br s, 2H), 3.04 (s, 6H). MS (EI) for C₁₂H₁₃N₇, found 256 (MH+).

### Example 25

### 4-[4-(dimethylamino)-1H-pyrazolo[3,4-d]pyrimidin-3-yl]phenol

The title compound was synthesized in a manner similar to Example 24 using the corresponding boronic acid substitution without protecting groups. ¹H-NMR (400 MHz, CD₃OD): δ 8.25 (s, 1H), 7.30 (t, 1H), 7.30-6.99 (m, 2H) 6.87 (dd, 1H), 2.94 (s, 6H). MS (EI) for C₁₃H₁₃N₅O, found 256.1 (MH+).

### Example 26

### 4-[4-(4-Methylpiperazin-1-yl)-1H-pyrrolo[2,3-b]pyridin-3-yl]pyridin-2-amine

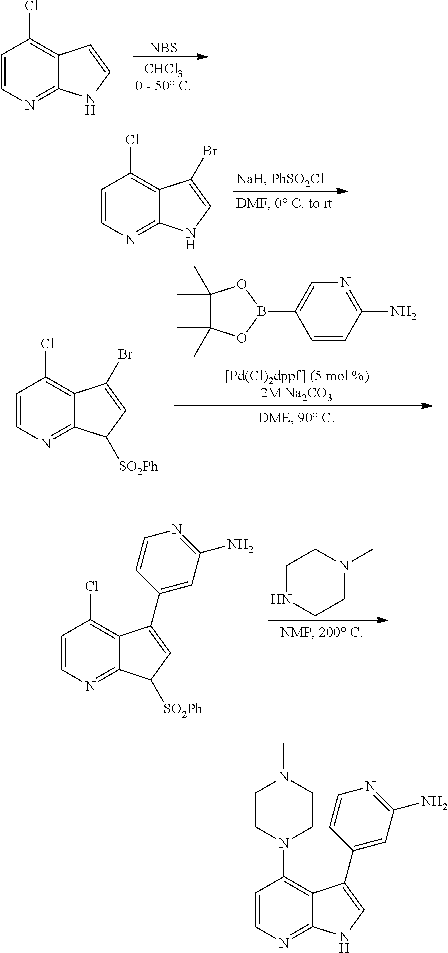

### 3-Bromo-4-chloro-1H-pyrrolo[2,3-b]pyridine

To a solution of 4-chloro-1H-pyrrolo[2,3-b]pyridine (1.003 g, 6.59 mmol) in chloroform (24 mL) was added portionwise N-bromosuccinimide (1.228 g, 6.90 mmol) at 0° C. After completion of addition, the cooling bath was removed. 30 min after stirring at room temperature, the mixture was heated at 50° C. for 1.5 h. The mixture was poured into 1M aqueous solution of K₂CO₃ (80 mL) and extracted with ethyl acetate (2×100 mL). The combined organic layers were washed with water and brine, and dried over MgSO₄. The title compound, 3-Bromo-4-chloro-1H-pyrrolo[2,3-b]pyridine, was isolated as a pale yellow solid (1.477 g, 97% yield), with purity determination by TLC. ¹H-NMR (400 MHz, DMSO-d6): δ 12.48 (br s, 1H), 8.22 (d, 1H), 7.83 (s, 1H), 7.24 (d, 1H). MS (EI) for C₇H₄BrClNO₂, found 230.9 (MH⁺, ⁷⁹Br), 232.9 (MH⁺, ⁸¹Br).

### 3-Bromo-4-chloro-1-(phenylsulfonyl)-1H-pyrrolo[2,3-b]pyridine

A two-neck 100 mL round bottom flask was charged with sodium hydride (60% oil dispersion; 378 mg, 9.4 mmol) and anhydrous dimethoxyethane (25 mL) under nitrogen. A solution of 3-bromo-4-chloro-1H-pyrrolo[2,3-b]pyridine (1.460 g, 6.30 mmol) in anhydrous dimethylformamide (8 mL) under nitrogen was added via cannula at 0° C. and stirred for 45 min. followed by dropwise addition of trimethylsilylethoxymethylchloride (90% tech; 0.90 mL, 7.05 mmol) via syringe. The reaction mixture was gradually warmed to room temperature and stirred for 16 h. The mixture was poured onto water (100 mL) and extracted with ethyl acetate (2×150 mL). The combined organic layers were washed with water (100 mL) and brine (80 mL), then dried over magnesium sulfate. After purification by flash chromatography (85:15 hexanes/ethyl acetate), the title compound was obtained as a white solid (2.61 g, quantitative yield). ¹H-NMR (400 MHz, CDCl₃): δ 8.31 (d, 1H), 8.20 (d, 2H), 7.85 (s, 1H), 7.61 (m, 1H), 7.53 (m, 2H), 7.22 (d, 1H). MS (EI) for C₁₃H₈BrClN₂O₂S, found 371 (MH⁺, ⁷⁹Br), 373 (MH⁺, ⁸¹Br).

### 4-[4-Chloro-1-(phenylsulfonyl)-1H-pyrrolo[2,3-b]pyridin-3-yl]pyridin-2-amine

The title compound was synthesized in a manner similar to the coupling in Example 22, wherein 3-bromo-4-methoxy-1-[[2-(trimethylsilyl)ethoxy]methyl]-1H-pyrazolo[3,4-d]pyrimidine was substituted with 3-bromo-4-chloro-1-(phenylsulfonyl)-1H-pyrrolo[2,3-b]pyridine. Upon purification by flash chromatography (94:4.5:0.5 dichloromethane/methanol/28% (w/w) ammonium hydroxide), the title compound was obtained as a solid (119 mg, 55% yield). ¹H-NMR (400 MHz, CDCl₃): δ 8.25 (m, 4H), 7.81 (m, 1H), 7.54 (m, 4H), 6.76 (s, 1H), 6.62 (s, 1H), 4.79 (br s, 2H). ¹³C-NMR (100 MHz, CDCl₃): δ 158.5, 147.7, 145.7, 137.9, 137.4, 134.8, 129.4, 128.5, 126.6, 125.6, 120.8, 119.3, 118.9, 116.1, 110.0. MS (EI) for C₁₈H₁₃ClN₄O₂S, found 385 (MH+).

### 4-[4-(4-Methylpiperazin-14)-1H-pyrrolo[2,3-b]pyridin-3-yl]pyridin-2-amine

4-[4-chloro-1-(phenylsulfonyl)-1H-pyrrolo[2,3-b]pyridin-3-yl]pyridin-2-amine (119 mg, 0.309 mmol) was treated with N-methylpiperazine (0.25 mL, 2.5 mmol) in N-methylpyrrolidinone (1 mL) and subjected to microwave irradiation for 30 min at 190° C. then at 200° C. for an additional 30 min. Upon cooling to room temperature, the mixture was concentrated under reduced pressure and azeotroped with acetonitrile. After purification by flash chromatography (91:8:1 dichloromethane/methanol/28% (w/w) ammonium hydroxide) followed by preparative HPLC (reverse-phase, 0.1% TFA in acetonitrile/0.05% TFA in water) the title compound was obtained as a white solid (25 mg, 26% yield). ¹H-NMR (400 MHz, DMSO-d6): δ 8.08 (d, 1H), 7.87 (d, 1H), 7.51 (s, 1H), 6.71 (m, 1H), 6.63 (m, 2H), 5.79 (s, 2H), 2.91 (m, 4H), 2.51 (s, 3H), 2.28 (m, 4H). MS (EI) for C₁₇H₂₀N₆, found 309 (MH⁺).

### Representative Conditions under Scheme 6

### Example 27

### 4-(4-(4-methylpiperazin-14)-1H-pyrrolo[2,3-b]pyridin-3-yl)pyridin-2-ol

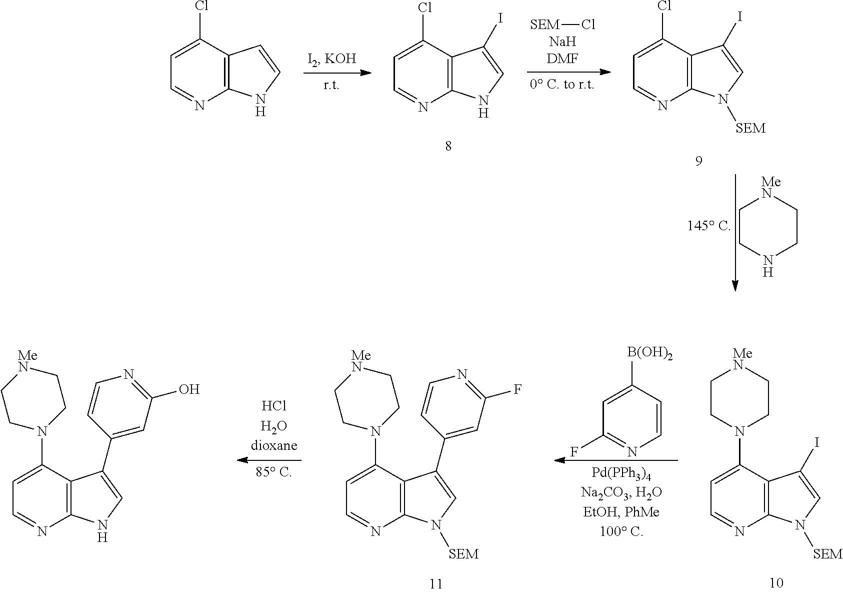

### 4-chloro-3-iodo-1H-pyrrolo[2,3-b]pyridine

To a solution of 4-chloro-7-azaindole (1.80 g, 11.8 mmol) in DMF (100 mL) was added potassium hydroxide (0.79 g, 14.1 mmol) at room temperature. The resulting mixture was stirred at room temperature for 10 minutes followed by addition of iodine (3.6 g, 14.1 mmol). After stirring at room temperature for 1.5 hour, the reaction mixture was poured onto a 50% aqueous Na₂S₂O₃ solution (150 mL). The resulting precipitate was collected by vacuum filtration to give the title compound (3.0 g, 92%). ¹H-NMR (400 MHz, DMSO-d₆): δ 8.21 (d, 1H), 7.82 (s, 1H), 7.20 (d, 1H). MS (ES) for C₇H₄ClIN₂, found 278.9 (MH+).

### 4-chloro-3-iodo-14(2-(trimethylsilyl)ethoxy)methyl)-1H-pyrrolo[2,3-b]pyridine

Sodium hydride (60% dispersion, 420 mg, 10.4 mmol) was added to a 0° C. solution of 4-chloro-3-iodo-1H-pyrrolo[2,3-b]pyridine (1.93 g, 6.9 mmol) in DMF (20 mL). The resulting mixture was stirred at 0° C. for 15 minutes prior to the addition of 2-(trimethylsilyl)ethoxymethyl chloride (1.85 mL, 10.4 mmol). The reaction mixture was allowed to warm to room temperature then stirred an additional 78 h followed by dilution with water. The resultant mixture was extracted three times with ethyl acetate. The combined organic extracts were washed with brine, dried with sodium sulfate, filtered and concentrated in vacuo. The crude mixture was purified by silica gel chromatography to give the title compound (2.2 g, 79%). ¹H-NMR (400 MHz, CDCl₃): δ 8.26 (d, 1H), 7.57 (s, 1H), 7.17 (d, 1H), 5.68 (s, 2H), 3.57 (t, 2H), 0.96 (t, 2H), 0.00 (s, 9H). MS (ES) for C₁₃H₁₈ClIN₂OSi, found 409.0 (MH+).

### 3-iodo-4-(4-methylpiperazin-1-yl)-1-((2-(trimethylsilyl)ethoxy)methyl)-1H-pyrrolo[2,3-b]pyridine

A solution of 4-chloro-3-iodo-1-((2-(trimethylsilyl)ethoxy)methyl)-1H-pyrrolo[2,3-b]pyridine (600 mg, 1.47 mmol) in N-methylpiperazine (10 mL) was stirred at 145° C. in a microwave reactor for 1 hour. The resultant mixture was then evaporated to dryness and purified by silica gel chromatography to give the title compound (330 mg, 48%). MS (ES) for C₁₈H₂₉IN₄OSi, found 473.0 (MH+).

### 3-(2-fluoropyridin-4-yl)-4-(4-methylpiperazin-1-((2-(trimethylsilyl)ethoxy)methyl)-1H-pyrrolo[2,3-b]pyridine

The title compound was coupled in a manner similar to Example 22, wherein 5-bromo-N,N-dimethyl-7-((2-(trimethylsilyl)ethoxy)methyl)-7H-pyrrolo[2,3-d]pyrimidin-4-amine was substituted with 3-iodo-4-(4-methylpiperazin-1-yl)-1-((2-(trimethylsilyl)ethoxy)methyl)-1H-pyrrolo[2,3-b]pyridine. The title compound was obtained in 37% yield. MS (ES) for C₂₃H₃₂FN₅OSi, found 442.3 (MH+).

### 4-(4-(4-methyl piperazin-14)-1H-pyrrolo[2,3-b]pyridin-3-yl)pyridin-2-ol

The title compound was synthesized in a manner similar to the SEM deprotection of Example 42, wherein 5-(2-fluoropyridin-4-yl)-N,N-dimethyl-7-((2-(trimethylsilyl)ethoxy)methyl)-7H-pyrrolo[2,3-d]pyrimidin-4-amine was substituted with 3-(2-fluoropyridin-4-yl)-4-(4-methylpiperazin-1-yl)-1-((2-(trimethylsilyl)ethoxy)methyl)-1H-pyrrolo[2,3-b]pyridine (11). Purification by preparative HPLC afforded the title compound as the trifluoroacetate salt (6 mg, 7%). ¹H-NMR (400 MHz, MeOH-d₄): δ 8.31 (d, 1H), 7.90 (s, 1H), 7.67 (d, 1H), 7.24 (d, 1H), 6.79 (dd, 1H), 6.72 (d, 1H), 3.99 (m, 2H), 3.54 (m, 2H), 3.11-3.27 (m, 4H), 2.94 (s, 3H). MS (ES) for C₁₇H₁₉N₅O, found 310.2 (MH+).

### Representative Conditions under Scheme 7

### Example 28

### 4-[4-(piperazin-1-yl)-1H-pyrazolo[3,4-d]pyrimidin-3-yl]pyridin-2-amine

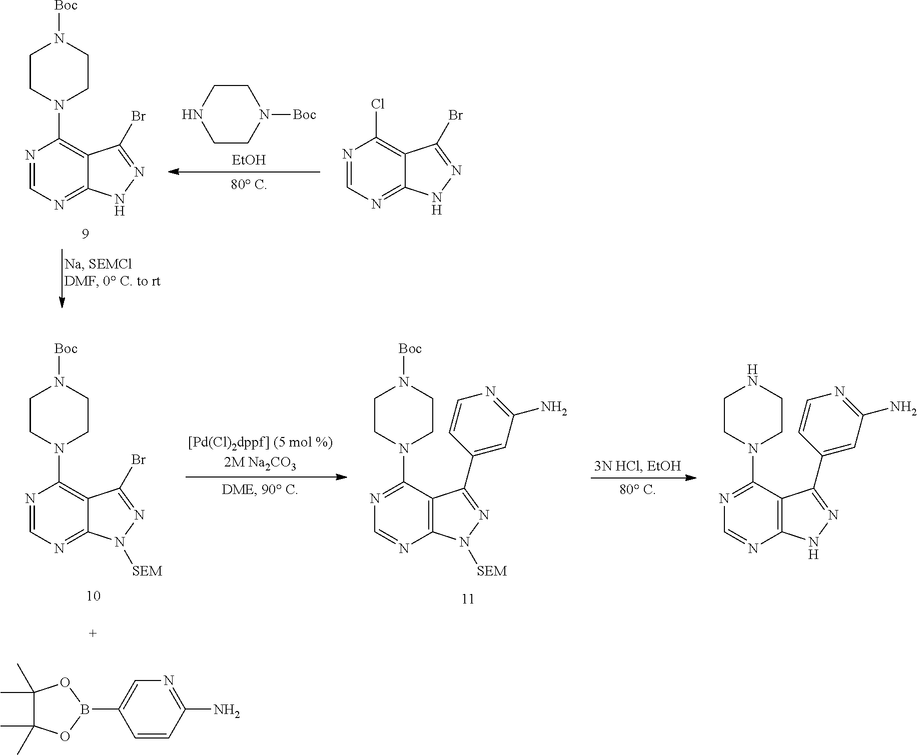

### tert-Butyl 4-(3-bromo-1H-pyrazolo[3,4-d]pyrimidin-4-yl)piperazine-1-carboxylate

The title compound was synthesized in a manner similar to Example 24, wherein dimethylamine was substituted with N-Boc-piperazine. After purification by flash chromatography (91:8:1 dichloromethane/methanol/28% (w/w) ammonium hydroxide), tert-Butyl 4-(3-bromo-1H-pyrazolo[3,4-d]pyrimidin-4-yl)piperazine-1-carboxylate was obtained as a low temperature melting solid (531 mg, 95% yield). ¹H-NMR (400 MHz, CDCl₃): δ 8.46 (s, 1H), 3.92 (m, 4H), 3.65 (m, 4H), 1.51 (s, 9H). MS (EI) for C₁₄H₁₉BrN₆O₂, found 384 (MH+).

### tert-butyl-4-[3-bromo-1-[[2-(trimethylsilyl)ethoxy]methyl]-1H-pyrazolo[3,4-d]pyrimidin-4-yl]piperazine-1-carboxylate

The title compound was synthesized in a manner similar to Example 22, wherein 3-bromo-4-methoxy-1H-pyrazolo[3,4-d]pyrimidine was substituted with 3-tert-Butyl 4-(3-bromo-1H-pyrazolo[3,4-d]pyrimidin-4-yl)piperazine-1-carboxylate. After purification by flash chromatography (8:2 hexanes/ethyl acetate), tert-butyl 4-[3-bromo-1-[[2-(trimethylsilyl)ethoxy]methyl]-1H-pyrazolo[3,4-d]pyrimidin-4-yl]piperazine-1-carboxylate was obtained as a white solid (611 mg, 86% yield). ¹H-NMR (400 MHz, CDCl₃): δ 8.44 (s, 1H), 5.73 (s, 2H), 3.88 (m, 4H), 3.71 (m, 2H), 3.67 (m, 4H), 1.53 (s, 9H), 0.97 (dd, 2H), 0.00 (s, 9H). MS (EI) for C₂₀H₃₃BrN₆O₃Si, found 513 (MH+).

### tert-butyl-4-[3-(2-aminopyridin-4-yl)-1-[[2-(trimethylsilyl)ethoxy]methyl]-1H-pyrazolo[3,4-d]pyrimidin-4-yl]piperazine-1-carboxylate

The title compound was synthesized in a manner similar to Example 22, wherein 3-bromo-4-methoxy-1-[[2-(trimethylsilyl)ethoxy]methyl]-1H-pyrazolo[3,4-d]pyrimidine was substituted with tert-Butyl-4-[3-bromo-1-[[2-(trimethylsilyl)ethoxy]methyl]-1H-pyrazolo[3,4-d]pyrimidin-4-yl]piperazine-1-carboxylate. After purification by flash chromatography (94:4.5:0.5 dichloromethane/methanol/28% (w/w) ammonium hydroxide), tert-butyl 443-(2-aminopyridin-4-yl)-1-[[2-(trimethylsilyl)ethoxy]methyl]-1H-pyrazolo[3,4-4 pyrimidin-4-yl]piperazine-1-carboxylate was obtained as a solid (69 mg, 72% yield). ¹H-NMR (400 MHz, CDCl₃): δ 8.55 (s, 1H), 8.24 (d, 1H), 6.99 (d, 1H), 6.91 (s, 1H), 5.84 (s, 2H), 4.72 (br s, 2H), 3.75 (dd, 2H), 3.52 and 3.42 (2×br m, 8H), 1.48 (s, 9H), 1.00 (dd, 2H), 0.00 (s, 9H). ¹³C-NMR (100 MHz, CDCl₃): δ 161.6, 160.3, 158.3, 156.4, 155.9, 150.2, 144.4, 115.2, 108.7, 101.4, 81.7, 76.7, 68.6, 29.8, 19.1, 0.00. MS (EI) for C₂₅H₃₈N₈O₃Si, found 527 (MH+).

### 4-[4-(piperazin-1-yl)-1H-pyrazolo[3,4-d]pyrimidin-3-yl]pyridin-2-amine

A solution of tert-Butyl 4-[3-(2-aminopyridin-4-yl)-1-[[2-(trimethylsilyl)ethoxy]methyl]-1H-pyrazolo[3,4-d]pyrimidin-4-yl]piperazine-1-carboxylate (69 mg, 0.131 mmol) in absolute ethanol (3 mL) was treated with a solution of 4 N HCl in 1,4-dioxane (0.3 mL) at 90° C. for 20 min. Upon removal of the Boc group, 6 N hydrochloric acid (0.1 mL) was added and the mixture was heated to gentle reflux for 2 h, then at 80° C. for 16 h. Upon cooling to room temperature, the mixture was filtered through paper and the collected solid was rinsed with cold ethanol. The hydrochloride salt of 4-[4-(piperazin-1-yl)-1H-pyrazolo[3,4-d]pyrimidin-3-yl]pyridin-2-amine was isolated as a white solid (29 mg, 75% yield). ¹H-NMR (400 MHz, d₆-DMSO-d₆): peaks split (due to salt forms). MS (EI) for C₁₄H₁₆N₈, found 297 (MH+).

### Representative Conditions under Scheme 8

### Example 29

### 4-methoxy-3-(pyridin-4-yl)-1H-pyrazolo[3,4-d]pyrimidine

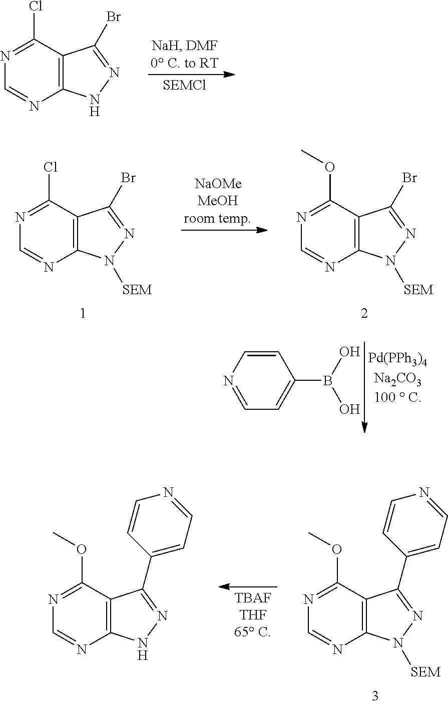

### 3-bromo-4-chloro-1-((2-(trimethylsilyl)ethoxy)methyl)-1H-pyrazolo[3,4-d]pyrimidine

Sodium hydride (60% dispersion, 720 mg, 18.1 mmol) was added to a cooled (0° C.) solution of 3-bromo-4-chloro-1H-pyrazolo[3,4-d]pyrimidine (3.10 g, 13.9 mmol) in DMF (20 mL). The resulting mixture was stirred at 0° C. for 15 minutes prior to the addition of 2-(trimethylsilyl)ethoxymethyl chloride (3.2 mL, 18 mmol). The reaction mixture was then allowed to warm to room temperature, stirred at room temperature for 18 hour, and diluted with water. The resulting mixture was extracted three times with ethyl acetate. The combined organic extracts were washed with brine, dried with sodium sulfate, filtered and concentrated. The crude mixture was purified by silica gel chromatography to give 3-bromo-4-chloro-14(2-(trimethylsilyl)ethoxy)methyl)-1H-pyrazolo[3,4-d]pyrimidine 1 (1.79 g, 35%). ¹H-NMR (400 MHz, CDCl₃): δ 8.84 (s, 1H), 5.83 (s, 2H), 3.71 (t, 2H), 0.97 (t, 2H), 0.06 (s, 9H).

### 3-bromo-4-methoxy-1-((2-(trimethylsilyl)ethoxy)methyl)-1H-pyrazolo[3,4-d]pyrimidine

Sodium methoxide (442 mg, 8.19 mmol) was added to a solution of 3-bromo-4-chloro-1-((2-(trimethylsilyl)ethoxy)methyl)-1H-pyrazolo[3,4-d]pyrimidine 1 (1.79 g, 5.46 mmol) in methanol (25 mL). The reaction mixture was stirred at room temperature for 2.5 hours. The resulting precipitate was collected by filtration and washed with water to give 3-bromo-4-methoxy-14(2-(trimethylsilyl)ethoxy)methyl)-1H-pyrazolo[3,4-d]pyrimidine 2 (1.5 g, 77%). ¹H-NMR (400 MHz, DMSO-d₆): δ 8.78 (s, 1H), 5.77 (s, 2H), 4.23 (s, 3H), 3.68 (t, 2H), 0.92 (t, 2H), 0.09 (s, 9H). MS (ES) for C₁₂H₁₉⁷⁹BrN₄O₂Si, found 359.1 (MH+) and C₁₂H₁₉⁸¹BrN₄O₂Si, found 361.1 (MH+).

### 4-methoxy-3-(pyridin-4-yl)-1-((2-(trimethylsilyl)ethoxy)methyl)-1H-pyrazolo[3,4-d]pyrimidine

3-bromo-4-methoxy-1-((2-(trimethylsilyl)ethoxy)methyl)-1H-pyrazolo[3,4-d]pyrimidine 2 (198 mg, 0.552 mmol) and pyridine-4-boronic acid (82 mg, 0.662 mmol) were combined in ethanol (0.5 mL), toluene (2.5 mL) and 2M sodium carbonate (0.70 mL). The resulting mixture was degassed with nitrogen for 15 minutes prior to the addition of tetrakis(triphenylphosphine)palladium(0) (32 mg, 0.028 mmol). The mixture was then stirred at 100° C. for 18 hours in a sealed tube then diluted with water. The resulting mixture was extracted three times with ethyl acetate. The combined organic extracts were then washed with brine, dried over sodium sulfate, filtered and concentrated. The crude mixture was purified by silica gel chromatography to give 4-methoxy-3-(pyridin-4-yl)-14(2-(trimethylsilyl)ethoxy)methyl)-1H-pyrazolo[3,4-d]pyrimidine 3 (138 mg, 70%) ¹H-NMR (400 MHz, CDCl₃): δ 8.77 (m, 2H), 8.68 (s, 1H), 8.10 (m, 2H), 5.90 (s, 2H), 3.74 (t, 2H), 1.00 (t, 2H), 0.03 (s, 9H). MS (ES) for C₁₇H₂₃N₆O₂Si, found 358.2 (MH+).

### 4-methoxy-3-(pyridin-4-yl)-1H-pyrazolo[3,4-d]pyrimidine

To solution of 4-methoxy-3-(pyridin-4-yl)-14(2-(trimethylsilyl)ethoxy)methyl)-1H-pyrazolo[3,4-d]pyrimidine 3 (138 mg, 0.387 mmol) in THF (5 mL) was added a 1.0 M solution of TBAF in THF (2.3 mL, 2.3 mmol). The resulting mixture was stirred at 65° C. for 15 hours then evaporated to dryness. The crude mixture was purified by preparative HPLC to afford 4-methoxy-3-(pyridin-4-yl)-1H-pyrazolo[3,4-d]pyrimidine (13 mg, 15%). ¹H-NMR (400 MHz, DMSO-d₆): δ 8.71 (m, 2H), 8.65 (s, H), 8.06 (m, 2H), 4.16 (s, 3H). MS (ES) for C₁₁H₉N₅O, found 228.1 (MH+).

Using analogous synthetic techniques as in Scheme 8 and Example 29 and substituting with the appropriate alternative starting materials, the following compounds were prepared. Alternative starting materials were obtained commercially unless otherwise indicated.

2-fluoro-5-[4-(methyloxy)-1H-pyrrolo[2,3-b]pyridin-3-yl]aniline. ¹H-NMR (400 MHz, CD₃OD): δ 8.08 (d, 1H), 7.20 (s, 1H), 7.10 (dd, 1H), 6.90 (m, 2H), 6.69 (d, 1H), 3.93 (s, 3H). MS (ES) for C₁₄H₁₂FN₃O, found 258.1 (MH+).

4-(methyloxy)-3-[2-(4-methylpiperazin-1-yl)pyridin-4-yl]-1H-pyrrolo[2,3-b]pyridine. ¹H-NMR (400 MHz, CD₃OD): δ 8.18 (d, 1H), 8.05 (d, 1H), 7.58 (s, 1H), 7.20 (s, 1H), 7.03 (d, 1H), 6.79 (d, 1H), 4.0 (s, 3H), 3.60 (br, 4H), 2.63 (br, 4H), 2.40 (s, 3H). MS (ES) for C₁₈H₂₁N₅O, found 324.2 (MH+).

4-chloro-6-[4-(methyloxy)-1H-pyrrolo[2,3-b]pyridin-3-yl]pyrimidin-2-amine. ¹H-NMR (400 MHz, CD₃OD): δ 8.17 (d, 1H), 8.04 (s, 1H), 7.46 (sd, 1H), 6.85 (d, 1H), 4.07 (s, 3H). MS (ES) for C₁₂H₁₀ClN₅O, found 276.1 (MH+).

4′-chloro-4-(methyloxy)-1H,1′H-3,5′-bipyrrolo[2,3-b]pyridine. ¹H-NMR (400 MHz, CD₃OD): δ 8.20 (s, 1H), 8.14 (d, 1H), 7.45 (d, 1H), 7.28 (s, 1H), 6.69 (d, 1H), 6.59 (d, 1H). MS (ES) for C₁₅H₁₁ClN₄O, found 299.1 (MH+).

### Representative Conditions under Scheme 9

### Example 30

### 4-methoxy-3-(pyridin-34)-1H-pyrazolo[3,4-d]pyrimidine

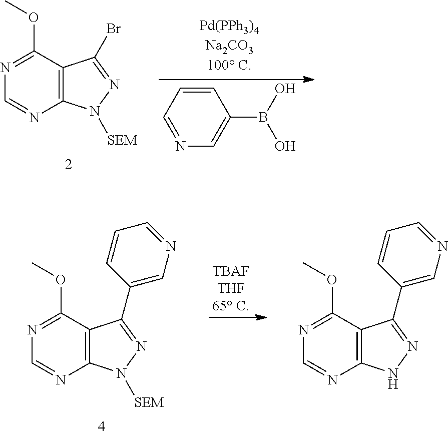

### 4-methoxy-3-(pyridin-34)-14(2-(trimethylsilyl)ethoxy)methyl)-1H-pyrazolo[3,4-d]pyrimidine

3-bromo-4-methoxy-1-((2-(trimethylsilyl)ethoxy)methyl)-1H-pyrazolo[3,4-d]pyrimidine 2 (100 mg, 0.279 mmol) and pyridine-3-boronic acid (45 mg, 0.363 mmol) were combined in ethanol (0.5 mL), toluene (2.5 mL) and 2M sodium carbonate (0.33 mL). The resulting mixture was degassed with nitrogen for 10 minutes prior to the addition of tetrakis(triphenylphosphine)palladium(0) (32 mg, 0.028 mmol). The mixture was then stirred at 100° C. for 2.5 hours in a sealed tube then diluted with water and extracted three times with ethyl acetate. The combined organic extracts were washed with brine, dried over sodium sulfate, filtered and concentrated. The crude mixture was purified by silica gel chromatography to give 4-methoxy-3-(pyridin-3-yl)-14(2-(trimethylsilyl)ethoxy)methyl)-1H-pyrazolo[3,4-d]pyrimidine 4 (76 mg, 77%) ¹H-NMR (400 MHz, CDCl₃): δ 9.37 (m, 1H), 8.71 (m, 1H), 8.67 (s, 1H), 8.42 (m, 1H), 7.45 (m, 1H), 5.89 (s, 2H), 4.24 (s, 3H), 3.75 (t, 2H), 1.00 (t, 2H), 0.03 (s, 9H). MS (ES) for C₁₇H₂₃N₅O₂Si, found 358.2 (MH+).

### 4-methoxy-3-(pyridin-3-yl)-1H-pyrazolo[3,4-d]pyrimidine

To solution of 4-methoxy-3-(pyridin-3-yl)-1-((2-(trimethylsilyl)ethoxy)methyl)-1H-pyrazolo[3,4-d]pyrimidine (76 mg, 0.216 mmol) in THF (5 mL) was added a 1.0 M solution of TBAF in THF (2.0 mL, 2.0 mmol). The resulting mixture was stirred at 65° C. for 15 hours then diluted with water and extracted three times with ethyl acetate. The combined organic extracts were washed with brine, dried with sodium sulfate, filtered, and concentrated. The crude mixture was purified by preparative HPLC to give 4-methoxy-3-(pyridin-3-yl)-1H-pyrazolo[3,4-d]pyrimidine (10 mg, 20%). ¹H-NMR (400 MHz, MeOH-d₄): δ 9.21 (s, 1H), 8.58 (m, 1H), 8.57 (s, 1H), 8.50 (m, 1H), 7.57 (m, 1H), 4.20 (s, 3H). MS (ES) for C₁₁H₉N₅O, found 228.1 (MH+).

### Example 31

### 3-(4-methoxy-1H-pyrazolo[3,4-d]pyrimidin-34)aniline

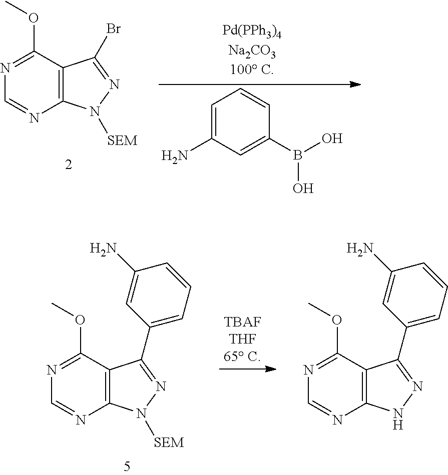

### 3-(4-methoxy-1-((2-(trimethylsilyl)ethoxy)methyl)-1H-pyrazolo[3,4-d]pyrimidin-3-yl)aniline

3-bromo-4-methoxy-1-((2-(trimethylsilyl)ethoxy)methyl)-1H-pyrazolo[3,4-d]pyrimidine 2 (100 mg, 0.279 mmol) and 3-aminophenylboronic acid (56.3 mg, 0.363 mmol) were combined in ethanol (0.5 mL), toluene (2.0 mL) and 2M sodium carbonate (0.33 mL). The resulting mixture was degassed with nitrogen for 10 minutes prior to the addition of tetrakis(triphenylphosphine)palladium(0) (32 mg, 0.028 mmol). The mixture was heated at 100° C. for 15 hours in a sealed tube and upon cooling diluted with water and extracted three times with ethyl acetate. The combined organic extracts were then washed with brine, dried over sodium sulfate, filtered and concentrated. The crude mixture was purified by silica gel chromatography to give 3-(4-methoxy-14(2-(trimethylsilyl)ethoxy)methyl)-1H-pyrazolo[3,4-d]pyrimidin-3-yl)aniline 5 (85 mg, 82%) ¹H-NMR (400 MHz, CDCl₃): δ 8.64 (s, 1H), 7.52 (m, 1H), 7.44 (m, 1H), 7.31 (m, 1H), 6.81 (m, 1H), 5.87 (s, 2H), 4.22 (s, 3H), 3.74 (t, 2H), 1.00 (t, 2H), 0.04 (s, 9H). MS (ES) for C₁₈H₂₅N₅O₂Si, found 372.2 (MH+).

### 3-(4-methoxy-1H-pyrazolo[3,4-d]pyrimidin-3-yl)aniline

To a solution of 3-(4-methoxy-14(2-(trimethylsilyl)ethoxy)methyl)-1H-pyrazolo[3,4-d]pyrimidin-3-yl)aniline (85 mg, 0.229 mmol) in THF (3.5 mL) was added a 1.0 M solution of TBAF in THF (3.0 mL, 3.0 mmol). The resulting mixture was stirred at 65° C. for 18 hours then diluted with water and extracted three times with ethyl acetate. The combined organic extracts were washed with brine, dried with sodium sulfate, filtered, and concentrated. The crude mixture was purified by silica gel chromatography. The resulting solid was then washed with chloroform to give 3-(4-methoxy-1H-pyrazolo[3,4-d]pyrimidin-3-yl)aniline (4 mg, 7%). ¹H-NMR (400 MHz, MeOH-d₄): δ 8.52 (s, 1H), 7.66 (m, 1H), 7.31 (m, 1H), 7.20 (t, 1H), 6.80 (m, 1H), 4.17 (s, 3H). MS (ES) for C₁₂H11N₅O, found 242.1 (MH+).

### Representative Conditions under Scheme 10

### Example 32

### 3-(2-aminopyrimidin-4-yl)-1H-pyrazolo[3,4-d]pyrimidin-4-amine

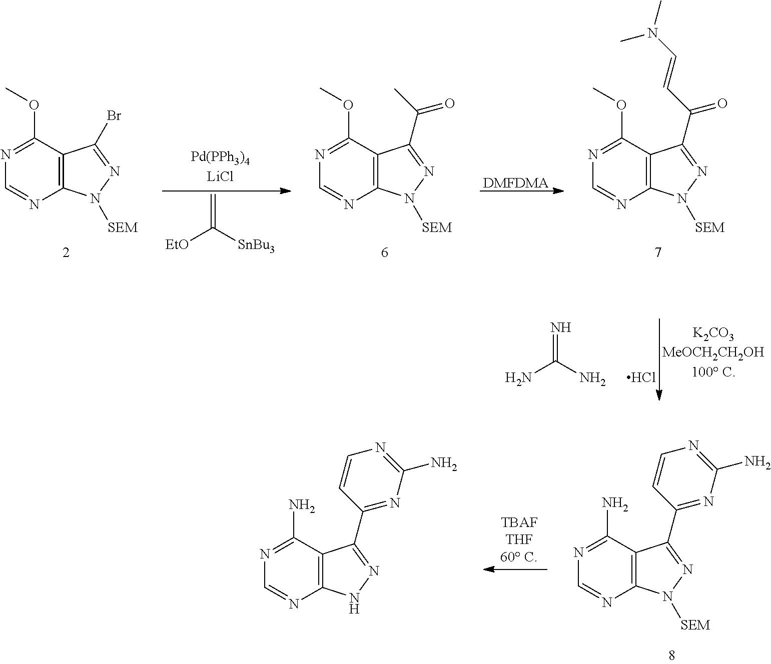

### 1-(4-methoxy-1-((2-(trimethylsilyl)ethoxy)methyl)-1H-pyrazolo[3,4-d]pyrimidin-3-yl)ethanone

To a degassed suspension of 3-bromo-4-methoxy-1-((2-(trimethylsilyl)ethoxy)methyl)-1H-pyrazolo[3,4-d]pyrimidine 2 (326 mg, 0.908 mmol) and lithium chloride (97 mg, 2.3 mmol) in DMF (8 mL) was added tributyl(1-ethoxyvinyl)stannane (0.47 mL, 1.4 mmol) and tetrakis(triphenylphosphine)palladium(0) (105 mg, 0.0900 mmol). The resulting mixture was stirred for 18 hours at 80° C. A 10% aqueous hydrochloric acid solution (10 mL) was then added to the reaction mixture and stirred for 1 hour at room temperature. The mixture was extracted three times with ethyl acetate. The combined organic extracts were washed with brine, dried over sodium sulfate, filtered and concentrated. The crude mixture was purified by silica gel chromatography to afford 1-(4-methoxy-14(2-(trimethylsilyl)ethoxy)methyl)-1H-pyrazolo[3,4-d]pyrimidin-3-yl)ethanone 6 (130 mg, 44%) ¹H-NMR (400 MHz, CDCl₃): δ 8.63 (s, 1H), 5.84 (s, 2H), 4.22 (s, 3H), 3.68 (t, 2H), 2.74 (s, 3H), 1.24 (t, 2H), 0.01 (s, 9H). MS (ES) for C₁₄H₂₂N₄O₃Si, found 323.2 (MH+).

### (E)-3-(dimethylamino)-1-(4-methoxy-1-((2-(trimethylsilyl)ethoxy)methyl)-1H-pyrazolo[3,4-d]pyrimidin-3-yl)prop-2-en-1-one

A mixture of 1-(4-methoxy-1-((2-(trimethylsilyl)ethoxy)methyl)-1H-pyrazolo[3,4-d]pyrimidin-3-yl)ethanone 6 (130 mg, 0.403 mmol) and N,N-dimethylformamide dimethyl acetal (0.330 mL, 2.48 mmol) in DMF (5 mL) was stirred at 90° C. for 3 hours then diluted with water. The resulting mixture was extracted three times with ethyl acetate. The combined organic extracts were washed with water, dried with sodium sulfate, filtered and concentrated to give (E)-3-(dimethylamino)-1-(4-methoxy-1-((2-(trimethylsilyl)ethoxy)methyl)-1H-pyrazolo[3,4-d]pyrimidin-3-yl)prop-2-en-1-one 7 (91 mg, 60%). MS (ES) for C₁₇H₂₇N₅O₃Si, found 378.2 (MH+).

### 3-(2-aminopyrimidin-4-yl)-1-((2-(trimethylsilyl)ethoxy)methyl)-1H-pyrazolo[3,4-d]pyrimidin-4-amine

A mixture of (E)-3-(dimethylamino)-1-(4-methoxy-1-((2-(trimethylsilyl)ethoxy)methyl)-1H-pyrazolo[3,4-d]pyrimidin-3-yl)prop-2-en-1-one 7 (91 mg, 0.24 mmol), guanidine hydrogen chloride (70 mg, 0.72 mmol) and potassium carbonate (166 mg, 1.20 mmol) in 2-methoxyethanol (5 mL) was stirred at 100° C. for 18 hours then diluted with water. The resulting mixture was extracted three times with ethyl acetate. The organic mixture was washed with brine, dried with sodium sulfate, filtered and concentrated to give 3-(2-aminopyrimidin-4-yl)-14(2-(trimethylsilyl)ethoxy)methyl)-1H-pyrazolo[3,4-d]pyrimidin-4-amine 8 (52 mg, 58%). MS (ES) for C₁₅H₂₂N₈OSi, found 359.2 (MH+).

### 3-(2-aminopyrimidin-4-yl)-1H-pyrazolo[3,4-d]pyrimidin-4-amine

To a solution of 3-(2-aminopyrimidin-4-yl)-14(2-(trimethylsilyl)ethoxy)methyl)-1H-pyrazolo[3,4-d]pyrimidin-4-amine (52 mg, 0.14 mmol) in THF (5.0 mL) was added a 1.0 M solution of TBAF in THF (2.0 mL, 2.0 mmol). The resulting mixture was stirred at 65° C. for 15 hours then evaporated to dryness. The crude mixture was purified by preparative HPLC to afford 3-(2-aminopyrimidin-4-yl)-1H-pyrazolo[3,4-d]pyrimidin-4-amine (5 mg, 16%). ¹H-NMR (400 MHz, DMSO-d₆): δ 8.44 (m, 2H), 7.39 (d, 1H). MS (ES) for C₉H₈N₈, found 229.1 (MH+).

### Example 33

### 3-(2-aminopyrimidin-4-yl)-N,N-dimethyl-1H-pyrazolo[3,4-b]pyridin-4-amine

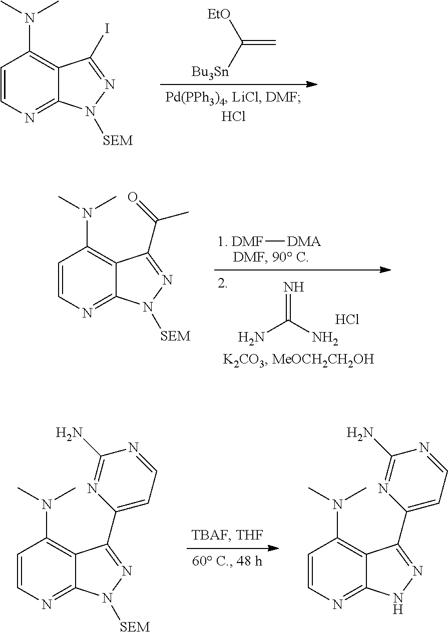

### 3-(2-aminopyrimidin-4-yl)-N,N-dimethyl-1H-pyrazolo[3,4-b]pyridin-4-amine

The title compound was synthesized in a manner similar to Example 32 beginning with the corresponding starting material from Example 21 to obtain the final compound 3-(2-aminopyrimidin-4-yl)-N,N-dimethyl-1H-pyrazolo[3,4-b]pyridin-4-amine ¹H-NMR (400 MHz, DMSO-d6): δ 8.32 (d, 1H), 8.13 (d, 1H), 6.92 (d, 1H), 6.72 (br s, 2H), 6.40 (d, 1H), 2.76 (s, 6H). MS (ES) for C₁₂H₁₃N₇, found 256.1 (MH+).

### Representative Conditions under Scheme 11

### Example 34

### 4-(4-methoxy-7H-pyrrolo[2,3-d]pyrimidin-5-yl)pyridin-2-amine

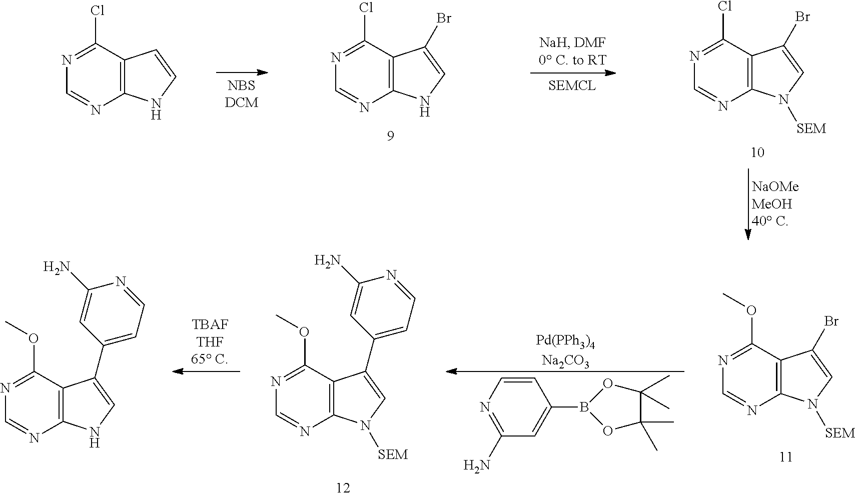

### 5-bromo-4-chloro-7H-pyrrolo[2,3-d]pyrimidine

A mixture of 4-chloro-7H-pyrrolo[2,3-d]pyrimidine (1.56 g, 10.2 mmol) and N-bromosuccinimide (2.35 g, 13.2 mmol) in dichloromethane (50 mL) was stirred at room temperature for 1.5 hours. The mixture was then concentrated and diluted with water. The resulting precipitate was collected by filtration to afford 5-bromo-4-chloro-7H-pyrrolo[2,3-d]pyrimidine 9 (1.45 g, 61%). ¹H-NMR (400 MHz, DMSO-d₆): δ 8.63 (s, 1H), 7.97 (s, 1H). MS (ES) for C₆H₃BrClN₃, found 233.9 (MH+).

5-bromo-4-chloro-7-((2-(trimethylsilyl)ethoxy)methyl)-7H-pyrrolo[2,3-d]pyrimidine

Sodium hydride (60% dispersion, 343 mg, 8.58 mmol) was added to a cooled (0° C.) solution of 5-bromo-4-chloro-7H-pyrrolo[2,3-d]pyrimidine 9 (1.00 g, 4.30 mmol) in DMF (25 mL). The resulting mixture was stirred at 0° C. for 15 minutes prior to the addition of 2-(trimethylsilyl)ethoxymethyl chloride (1.5 mL, 8.6 mmol). The reaction mixture was then allowed to warm to room temperature, stirred at room temperature for 18 h, and diluted with water. The resulting mixture was extracted three times with ethyl acetate. The combined organic extracts were washed with brine, dried with sodium sulfate, filtered and concentrated. The crude mixture was purified by silica gel chromatography to give 5-bromo-4-chloro-7-((2-(trimethylsilyl)ethoxy)methyl)-7H-pyrrolo[2,3-d]pyrimidine 10 (1.0 g, 64%). ¹H-NMR (400 MHz, CDCl₃): δ 8.70 (s, 1H), 7.48 (s, 1H), 5.67 (s, 2H), 3.57 (t, 2H), 0.96 (t, 2H), 0.04 (s, 9H). MS (ES) for C₁₂H₁₇BrClN₃OSi, found 364.0 (MH+).

### 5-bromo-4-methoxy-7-((2-(trimethylsilyl)ethoxy)methyl)-7H-pyrrolo[2,3-d]pyrimidine

Sodium methoxide (0.22 g, 4.1 mmol) was added to a suspension of 5-bromo-4-chloro-7-((2-(trimethylsilyl)ethoxy)methyl)-7H-pyrrolo[2,3-d]pyrimidine 10 (1.00 g, 2.75 mmol) in methanol (25 mL). The reaction mixture was stirred at 40° C. for 2.5 hours then diluted with water. The resulting precipitate was collected by filtration and washed with water to give 5-bromo-4-methoxy-7-((2-(trimethylsilyl)ethoxy)methyl)-7H-pyrrolo[2,3-d]pyrimidine 11 (660 mg, 67%). ¹H-NMR (400 MHz, DMSO-d₆): δ 8.53 (s, 1H), 7.27 (s, 1H), 5.64 (s, 2H), 4.22 (s, 3H), 3.58 (t, 2H), 0.98 (t, 2H), 0.07 (s, 9H). MS (ES) for C₁₃H₂₀BrN₃O₂Si, found 358.1 (MH+).

### 4-(4-methoxy-7((2-(trimethylsilyl)ethoxy)methyl)-7H-pyrrolo[2,3-d]pyrimidin-5-yl)pyridin-2-amine

5-bromo-4-methoxy-7-((2-(trimethylsilyl)ethoxy)methyl)-7H-pyrrolo[2,3-d]pyrimidine 11 (120 mg, 0.335 mmol) and 2-aminopyridine-4-boronic acid pinacol ester (110 mg, 0.500 mmol) were combined in ethanol (1.0 mL), toluene (3.5 mL) and 2M sodium carbonate (0.50 mL). The resulting mixture was degassed with nitrogen for 15 minutes prior to the addition of tetrakis(triphenylphosphine)palladium(0) (39 mg, 0.034 mmol). The mixture was then stirred at 100° C. for 18 hours in a sealed tube then diluted with water. The resulting mixture was extracted three times with ethyl acetate. The combined organic extracts were then washed with brine, dried over sodium sulfate, filtered and concentrated. The crude mixture was purified by silica gel chromatography to give 4-(4-methoxy-7-((2-(trimethylsilyl)ethoxy)methyl)-7H-pyrrolo[2,3-d]pyrimidin-5-yl)pyridin-2-amine 12 (31 mg, 25%). ¹H-NMR (400 MHz, CDCl₃): δ 8.57 (s, 1H), 8.14 (d, 1H), 7.46 (s, 1H), 7.03 (m, 1H), 6.92 (s, 1H), 5.71 (s, 2H), 4.51 (s, 2H), 4.18 (s, 3H), 3.61 (t, 2H), 0.98 (t, 2H), 0.04 (s, 9H). MS (ES) for C₁₈H₂₅N₅O₂Si, found 372.2 (MH+).

### 4-(4-methoxy-7H-pyrrolo[2,3-d]pyrimidin-5-yl)pyridin-2-amine

To a solution of 4-(4-methoxy-7-((2-(trimethylsilyl)ethoxy)methyl)-7H-pyrrolo[2,3-d]pyrimidin-5-yl)pyridin-2-amine 12 (43 mg, 0.12 mmol) in THF (3.0 mL) was added a 1.0 M solution of TBAF in THF (1.0 mL, 1.0 mmol). The resulting mixture was stirred at 60° C. for 20 hours then evaporated to dryness. The crude mixture was purified by preparative HPLC to give 4-(4-methoxy-7H-pyrrolo[2,3-d]pyrimidin-5-yl)pyridin-2-amine (7 mg, 24%). ¹H-NMR (400 MHz, MeOH-₄): δ 8.41 (s, 1H), 7.81 (d, 1H), 7.72 (s, 1H), 7.18 (d, 1H), 7.07 (m, 1H), 4.15 (s, 3H). MS (ES) for C₁₂H₁₁N₅O, found 242.1 (MH+).

### Representative Conditions Under Scheme 12

### Example 35

### 5-(2-aminopyrimidin-4-yl)-N,N-dimethyl-7H-pyrrolo[2,3-d]pyrimidin-4-amine

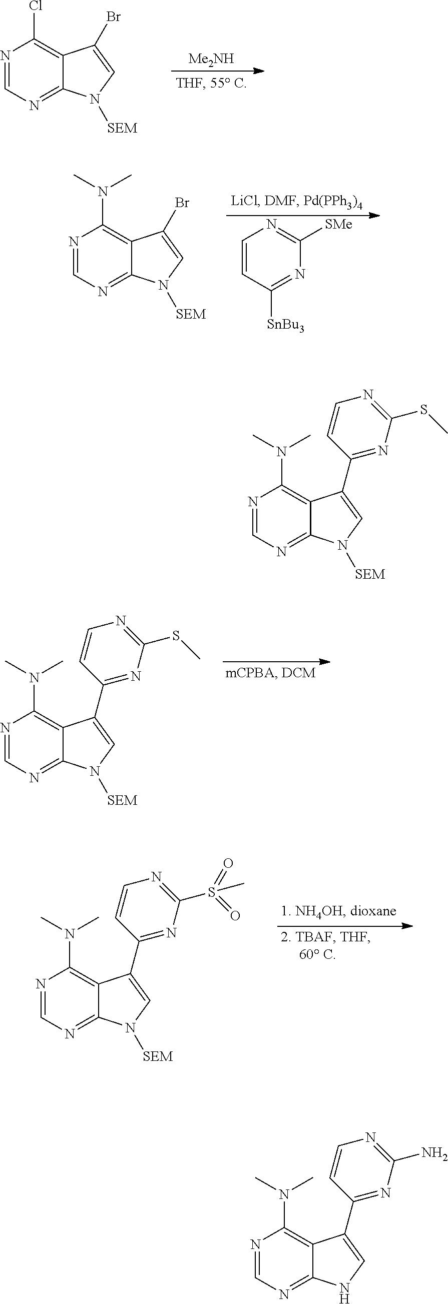

### 5-bromo-N,N-dimethyl-7-((2-(trimethylsilyl)ethoxy)methyl)-7H-pyrrolo[2,3-d]pyrimidin-4-amine

In a sealed tube 5-bromo-4-chloro-7-((2-(trimethylsilyl)ethoxy)methyl)-7H-pyrrolo[2,3-d]pyrimidine (0.92 g, 2.54 mmol), dimethylamine (20 mL, 2.0 M in THF), and THF (30 mL) were combined and heated at 55° C. overnight. The resulting reaction mixture was concentrated to give 5-bromo-N,N-dimethyl-7-((2-(trimethylsilyl)ethoxy)methyl)-7H-pyrrolo[2,3-c]pyrimidin-4-amine. MS (ES) for C₁₄H₂₃BrN₄OSi, found 371.0 (MH+).

### N,N-dimethyl-5-(2-(methylthio)pyrimidin-4-yl)-7-((2-(trimethylsilyl)ethoxy)methyl)-7H-pyrrolo[2,3-d]pyrimidin-4-amine

A mixture of 5-bromo-N,N-dimethyl-7-((2-(trimethylsilyl)ethoxy)methyl)-7H-pyrrolo[2,3-d]pyrimidin-4-amine (0.10 g, 0.27 mmol) and lithium chloride (0.028 g, 0.67 mmol) were suspended in DMF (4 mL) under nitrogen and degassed for 15 minutes. Palladium tetrakis(triphenylphosphine) (0.031 g, 0.03 mmol) and 2-(methylthio)-4-(tributylstannyl)pyrimidine (0.13 mL, 0.32 mmol) were added to the solution and the mixture was heated at 80° C. for 18 hours. The crude product was filtered through celite, washed with a 5% aqueous LiCl solution, and extracted with ethyl acetate. The resulting organic layer was washed with brine, dried over sodium sulfate, filtered and concentrated. The crude material was purified by flash chromatography (90% Hexane: 10% Ethyl acetate) to give N,N-dimethyl-5-(2-(methylthio)pyrimidin-4-yl)-7-((2-(trimethylsilyl)ethoxy)methyl)-7H-pyrrolo[2,3-d]pyrimidin-4-amine. MS (ES) for C₁₉H₂₈N₆OSSi, found 417.2 (MH+).

N,N-dimethyl-5-(2-(methylsulfonyl)pyrimidin-4-O-7-((2-(trimethylsilyl)ethoxy)methyl)-7H-pyrrolo[2,3-d]pyrimidin-4-amine

N,N-dimethyl-5-(2-(methylthio)pyrimidin-4-yl)-7-((2-(trimethylsilyl)ethoxy)methyl)-7H-pyrrolo[2,3-d]pyrimidin-4-amine (0.05 g, 0.12 mmol) and mCPBA (0.05 g, 0.24 mmol) in DCM (2 mL) were combined and stirred at room temperature for 2 hours. The resulting reaction mixture was washed with aqueous saturated NaHCO₃, extracted with DCM, and the organic layers were dried over sodium sulfate, filtered and concentrated to yield N,N-dimethyl-5-(2-(methylsulfonyl)pyrimidin-4-yl)-7-((2-(trimethylsilyl)ethoxy)methyl)-7H-pyrrolo[2,3-d]pyrimidin-4-amine. MS (ES) for C₁₉H₂₈N₆O₃SSi, found 449.2 (MH+).

### 5-(2-aminopyrimidin-4-yl)-N,N-dimethyl-7-((2-(trimethylsilyl)ethoxy)methyl)-7H-pyrrolo[2,3-d]pyrimidin-4-amine

N,N-dimethyl-5-(2-(methylsulfonyl)pyrimidin-4-yl)-7-((2-(trimethylsilyl)ethoxy)methyl)-7H-pyrrolo[2,3-d]pyrimidin-4-amine (0.54 g, 1.20 mmol), conc. ammonium hydroxide (3 mL) and dioxane (5 mL) were combined and stirred at room temperature for 24 hours. Water was added to the reaction mixture which was then partitioned into ethyl acetate and water. The crude product was extracted with ethyl acetate. The organic extracts were dried over sodium sulfate and concentrated. The resulting mixture was purified by flash chromatography (hexane:ethyl acetate 75:25 to 60:40) to yield 5-(2-aminopyrimidin-4-yl)-N,N-dimethyl-7-((2-(trimethylsilyl)ethoxy)methyl)-7H-pyrrolo[2,3-d]pyrimidin-4-amine. MS (ES) for C₁₈H₂₇N₇OSi, found 386 (MH+).

### 5-(2-aminopyrimidin-4-yl)-N,N-dimethyl-7H-pyrrolo[2,3-d]pyrimidin-4-amine

5-(2-aminopyrimidin-4-yl)-N,N-dimethyl-7-((2-(trimethylsilyl)ethoxy)methyl)-7H-pyrrolo[2,3-d]pyrimidin-4-amine (200 mg, mmol) was combined with TBAF (0.6 mL, 2.07 mmol) and THF (10 mL). The crude mixture was concentrated then purified by preparative HPLC to afford 5-(2-aminopyrimidin-4-yl)-N,N-dimethyl-7H-pyrrolo[2,3-c]pyrimidin-4-amine (10 mg, 5%). ¹H-NMR (400 MHz, CD₃OD): δ 8.23 (s, 1H), 8.21 (d, 1H), 7.66 (s, 1H), 6.92 (d, 1H), 2.98 (s, 6H). MS (ES) for C₁₂H₁₃N₇ found 256.1 (MH+).

### Representative Conditions under Scheme 13

### Example 36

### 3-(2-aminopyrimidin-4-yl)-N-isobutyl-1H-pyrrolo[2,3-b]pyridin-4-amine

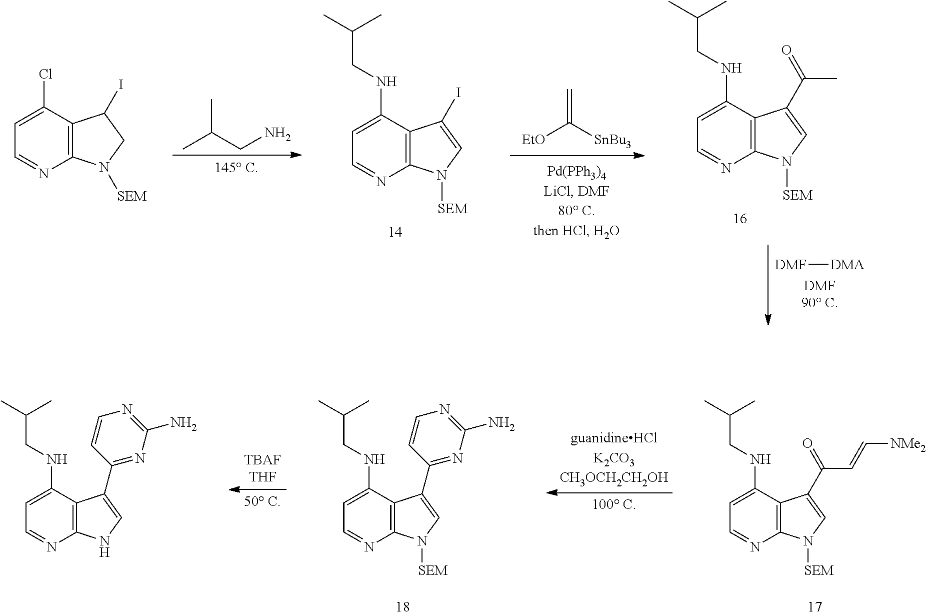

### 3-iodo-N-isobutyl-14(2-(trimethylsilyl)ethoxy)methyl)-1H-pyrrolo[2,3-b]pyridin-4-amine (14)

The title compound was synthesized in a manner similar to wherein N-methylpiperazine was substituted with isobutylamine utilizing microwave irradiation for SnAr. The title compound was obtained after flash chromatography in 52% yield. ¹H-NMR (400 MHz, CDCl₃): δ 8.08 (d, 1H), 7.32 (s, 1H), 6.23 (d, 1H), 5.97 (m, 1H), 5.61 (s, 2H), 3.58 (t, 2H), 3.14 (t, 2H), 2.09 (m, 1H), 1.13 (d, 6H), 0.96 (t, 2H), 0.00 (s, 9H). MS (ES) for C₁₇H₂₈IN₃OSi, found 446.1 (MH+).

### 1-(4-(isobutylamino)-1-((2-(trimethylsilyl)ethoxy)methyl)-1H-pyrrolo[2,3-b]pyridin-3-yl)ethanone (16)

The title compound was synthesized in a manner similar to Example 1, wherein 3-bromo-N,N-dimethyl-1-[[2-(trimethylsilyl)ethoxy]methyl]-1H-pyrazolo[3,4-d]pyrimidin-4-amine was substituted with 3-iodo-N-isobutyl-1-((2-(trimethylsilyl)ethoxy)methyl)-1H-pyrrolo[2,3-b]pyridin-4-amine (14). The title compound was obtained after flash chromatography in quantitative yield. ¹H-NMR (400 MHz, CDCl₃): δ 8.73 (m, 1H), 8.00 (d, 1H), 7.81 (s, 1H), 6.20 (d, 1H), 5.63 (s, 2H), 3.60 (t, 2H), 3.06 (t, 2H), 2.54 (s, 3H), 2.05 (m, 1H), 1.07 (d, 6H), 0.91 (t, 2H), 0.00 (s, 9H). MS (ES) for C₁₉H₃₁N₃O₂Si, found 362.3 (MH+).

### (E)-3-(dimethylamino)-1-(4-(isobutylamino)-14(2-(trimethylsilyl)ethoxy)methyl)-1H-pyrrolo[2,3-b]pyridin-3-yl)prop-2-en-1-one (17)

The title compound was synthesized in a manner similar to Example 1, wherein 1-(4-(dimethylamino)-1-((2-(trimethylsilyl)ethoxy)methyl)-1H-pyrazolo[3,4-d]pyrimidin-3-yl)ethanone was substituted with 1-(4-(isobutylamino)-1-((2-(trimethylsilyl)ethoxy)methyl)-1H-pyrrolo[2,3-b]pyridin-3-yl)ethanone (16). The crude product was carried onto the next step without any purification. MS (ES) for C₂₂H₃₆N₄O₂Si, found 417.3 (MH+).

### 3-(2-aminopyrimidin-4-yl)-N-isobutyl-14(2-(trimethylsilyl)ethoxy)methyl)-1H-pyrrolo[2,3-b]pyridin-4-amine (18)

The title compound was synthesized in a manner similar to Example 1, wherein (E)-3-(dimethylamino)-1-(4-(dimethylamino)-1-((2-(trimethylsilyl)ethoxy)methyl)-1H-pyrazolo[3,4-d]pyrimidin-3-yl)prop-2-en-1-one was substituted with (E)-3-(dimethylamino)-1-(4-(isobutylamino)-1-((2-(trimethylsilyl)ethoxy)methyl)-1H-pyrrolo[2,3-b]pyridin-3-yl)prop-2-en-1-one (17). The title compound was obtained in 67% yield after flash chromatography. MS (ES) for C₂₁H₃₂N₆OSi, found 413.3 (MH+).

### 3-(2-aminopyrimidin-4-yl)-N-isobutyl-1H-pyrrolo[2,3-b]pyridin-4-amine

To a solution of 3-(2-aminopyrimidin-4-yl)-N-isobutyl-1-((2-(trimethylsilyl)ethoxy)methyl)-1H-pyrrolo[2,3-b]pyridin-4-amine (222 mg, 0.539 mmol) in ethanol (2.5 mL) was added 6M HCl (1.5 mL). The resulting mixture was stirred at 100° C. for 24 hours before evaporated to dryness. The crude product was purified by preparative HPLC to give the title compound (17 mg, 11%). 2 ¹H-NMR (400 MHz, MeOH-d₄): δ 8.11 (d, 1H), 7.94 (s, 1H), 7.81 (d, 1H), 7.11 (d, 1H), 6.64 (d, 1H), 3.25 (d, 2H), 2.07 (m, 1H), 1.02 (d, 6H). MS (ES) for C₁₆H₁₈N₆, found 283.2 (MH+).

### Example 37

### 3-(2-aminopyrimidin-4-yl)-N-ethyl-1H-pyrrolo[2,3-b]pyridin-4-amine

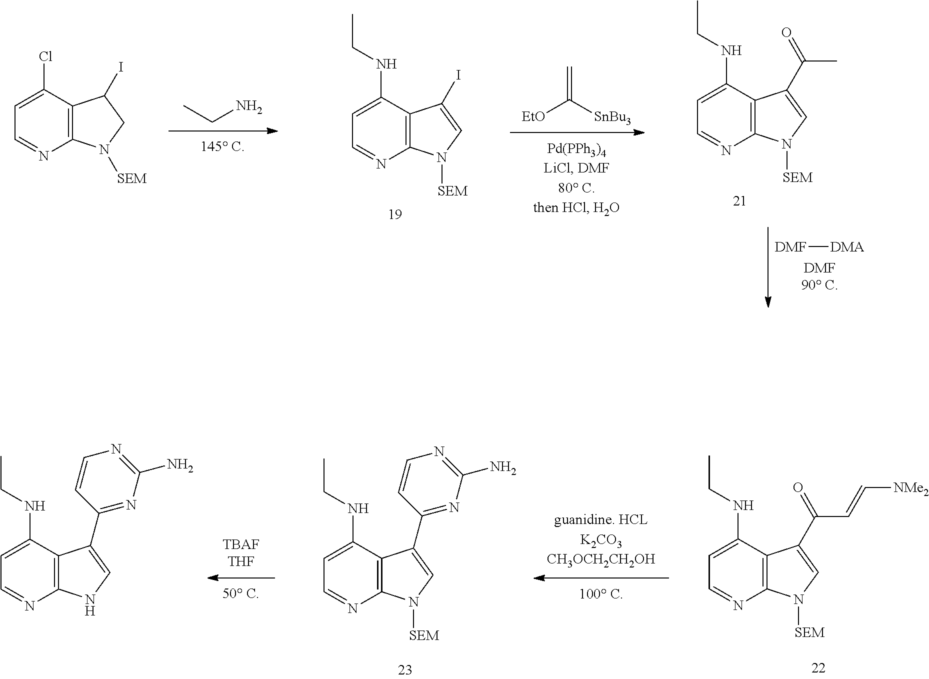

### N-ethyl-3-iodo-1-((2-(trimethylsilyl)ethoxy)methyl)-1H-pyrrolo[2,3-b]pyridin-4-amine (19)

The title compound was synthesized in a manner similar to Example 36 utilizing microwave irradiation for SnAr wherein N-methylpiperazine was substituted with a 70 wt % aqueous solution of ethylamine. The title compound was obtained after flash chromatography in 43% yield. ¹H-NMR (400 MHz, CDCl₃): δ 8.10 (d, 1H), 7.22 (s, 1H), 6.24 (d, 1H), 5.84 (m, 1H), 5.61 (s, 2H), 3.58 (t, 2H), 3.37 (m, 2H), 1.43 (t, 3H), 0.96 (t, 2H), 0.00 (s, 9H). MS (ES) for C₁₅H₂₄IN₃OSi, found 418.1 (MH+).

### 1-(4-(ethylamino)-1-((2-(trimethylsilyl)ethoxy)methyl)-1H-pyrrolo[2,3-b]pyridin-3-yl)ethanone (21)

The title compound was synthesized in a manner similar to Example 24, wherein 3-bromo-N,N-dimethyl-1-[[2-(trimethylsilyl)ethoxy]methyl]-1H-pyrazolo[3,4-b]pyrimidin-4-amine was substituted with N-ethyl-3-iodo-1-((2-(trimethylsilyl)ethoxy)methyl)-1H-pyrrolo[2,3-b]pyridin-4-amine (19). The title compound was obtained after flash chromatography in 97% yield. ¹H-NMR (400 MHz, CDCl₃): δ 8.65 (m, 1H), 8.06 (d, 1H), 7.86 (s, 1H), 6.25 (d, 1H), 5.68 (s, 2H), 3.64 (t, 2H), 3.33 (m, 2H), 2.58 (s, 3H), 1.43 (t, 3H), 0.97 (t, 2H), 0.00 (s, 9H). MS (ES) for C₁₇H₂₇N₃O₂Si, found 334.2 (MH+).

### (E)-3-(dimethylamino)-1-(4-(ethylamino)-1-((2-(trimethylsilyl)ethoxy)methyl)-1H-pyrrolo[2,3-b]pyridin-3-yl)prop-2-en-1-one (22)

The title compound was synthesized in a manner similar to Example 1, wherein 1-(4-(dimethylamino)-1-((2-(trimethylsilyl)ethoxy)methyl)-1H-pyrazolo[3,4-d]pyrimidin-3-yl)ethanone was substituted with 1-(4-(ethylamino)-1-((2-(trimethylsilyl)ethoxy)methyl)-1H-pyrrolo[2,3-b]pyridin-3-yl)ethanone (21). The crude product was carried onto the next step without any purification.

### 3-(2-aminopyrimidin-4-yl)-N-ethyl-1-((2-(trimethylsilyl)ethoxy)methyl)-1H-pyrrolo[2,3-b]pyridin-4-amine (23)

The title compound was synthesized in a manner similar Example 1, wherein (E)-3-(dimethylamino)-1-(4-(dimethylamino)-1-((2-(trimethylsilyl)ethoxy)methyl)-1H-pyrazolo[3,4-d]pyrimidin-3-yl)prop-2-en-1-one was substituted with (E)-3-(dimethylamino)-1-(4-(ethylamino)-14(2-(trimethylsilyl)ethoxy)methyl)-1H-pyrrolo[2,3-b]pyridin-3-yl)prop-2-en-1-one (22). The title compound was obtained in 62% yield after flash chromatography. ¹H-NMR (400 MHz, CDCl₃): δ 9.34 (m, 1H), 8.25 (d, 1H), 8.09 (d, 1H), 7.77 (s, 1H), 7.03 (d, 1H), 6.27 (d, 1H), 5.70 (s, 2H), 4.95 (s, 2H), 3.63 (t, 2H), 3.38 (m, 2H), 1.52 (t, 3H), 0.98 (t, 2H), 0.00 (s, 9H). MS (ES) for C₁₉H₂₈N₆OSi, found 385.2 (MH+).

### 3-(2-aminopyrimidin-4-yl)-N-ethyl-1H-pyrrolo[2,3-b]pyridin-4-amine

The title compound was synthesized in a manner similar to Example 22, wherein 3-(2-aminopyrimidin-4-yl)-N-isobutyl-14(2-(trimethylsilyl)ethoxy)methyl)-1H-pyrrolo[2,3-b]pyridin-4-amine was substituted with 3-(2-aminopyrimidin-4-yl)-N-ethyl-14(2-(trimethylsilyl)ethoxy)methyl)-1H-pyrrolo[2,3-b]pyridin-4-amine (23). The title compound was obtained in 29% yield after purification by preparative HPLC. ¹H-NMR (400 MHz, MeOH-d₄): δ 8.09 (d, 1H), 7.90 (s, 1H), 7.83 (d, 1H), 7.08 (d, 1H), 6.28 (d, 1H), 3.40 (q, 2H), 1.40 (t, 3H). MS (ES) for C₁₃H₁₄N₆, found 255.1 (MH+).

### Example 38

### 4-(4-ethoxy-1H-pyrrolo[2,3-b]pyridin-3-O-6-methylpyrimidin-2-amine

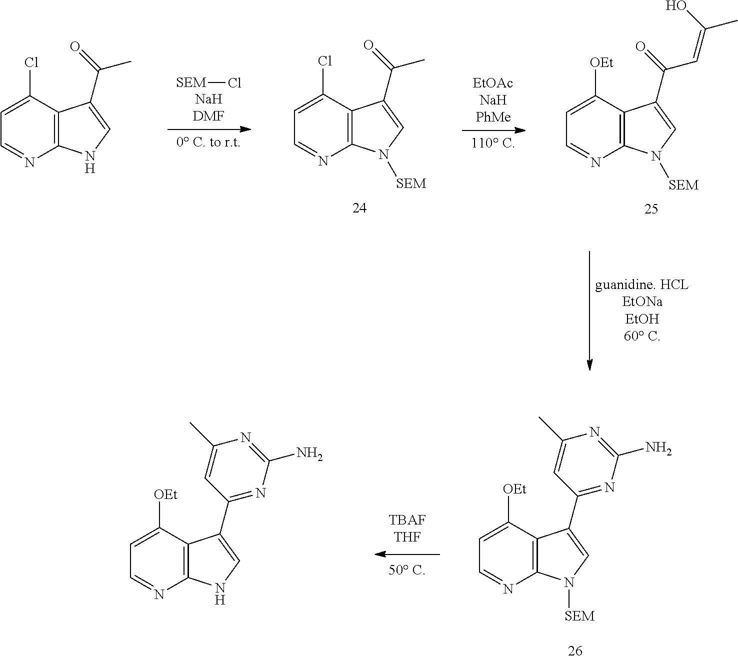

### 1-(4-chloro-1-((2-(trimethylsilyl)ethoxy)methyl)-1H-pyrrolo[2,3-b]pyridin-3-yl)ethanone (24)

The title compound was synthesized in a manner similar to Example 22, wherein 4-chloro-3-iodo-1H-pyrrolo[2,3-b]pyridine was substituted with 1-(4-chloro-1H-pyrrolo[2,3-b]pyridin-3-yl)ethanone. The title compound was obtained in 87% yield after flash chromatography. ¹H-NMR (400 MHz, CDCl₃): δ 8.30 (d, 1H), 8.07 (s, 1H), 7.32 (d, 1H), 5.76 (d, 2H), 3.62 (t, 2H), 2.65 (s, 3H), 0.976 (t, 2H), 0.00 (s, 9H). MS (ES) for C₁₅H₂₁ClN₂O₂Si, found 325.1 (MH+).

### (Z)-1-(4-ethoxy-1-((2-(trimethylsilyl)ethoxy)methyl)-1H-pyrrolo[2,3-b]pyridin-3-O-3-hydroxybut-2-en-1-one (25)

A mixture of 1-(4-chloro-1-((2-(trimethylsilyl)ethoxy)methyl)-1H-pyrrolo[2,3-b]pyridin-3-yl)ethanone (206 mg, 0.636 mmol) and sodium hydride (60% dispersion, 64 mg, 1.6 mmol) in ethyl acetate (2.5 mL) and toluene (6 mL) was stirred at reflux for 24 hours. The resulting mixture was then cooled to room temperature, diluted with saturated ammonium chloride, and extracted three times with ethyl acetate. The combined organic extracts were washed with brine, dried over sodium sulfate, filtered and concentrated. The crude product was purified by flash chromatography to give the title compound (100 mg, 43%). MS (ES) for C₁₉H₂₈N₂O₄Si, found 377.2 (MH+)

### 4-(4-ethoxy-1-((2-(trimethylsilyl)ethoxy)methyl)-1H-pyrrolo[2,3-b]pyridin-34)-6-methylpyrimidin-2-amine (26)

Guanidine hydrochloride (127 mg, 1.33 mmol) and sodium ethoxide (20 wt % in ethanol, 0.50 mL, 1.3 mmol) in ethanol (3 mL) was stirred at room temperature for 15 min. The resulting mixture was filtered to give a guanidine free base solution. To a solution of (Z)-1-(4-ethoxy-14(2-(trimethylsilyl)ethoxy)methyl)-1H-pyrrolo[2,3-b]pyridin-3-yl)-3-hydroxybut-2-en-1-one (100 mg, 0.266 mmol) in ethanol (5 mL) was then added the above guanidine solution. The mixture was then stirred at 60° C. for 15 hours then evaporated to dryness. The crude product was then purified by flash chromatography to give the title compound (52 mg, 49%). ¹H-NMR (400 MHz, CDCl₃): δ 8.30 (d, 1H), 8.08 (s, 1H), 7.53 (s, 1H), 6.70 (d, 1H), 5.75 (s, 2H), 4.94 (s, 2H), 4.33 (q, 2H), 3.63 (t, 2H), 2.45 (s, 3H), 1.63 (t, 3H), 0.99 (t, 2H). MS (ES) for C₂₀H₂₉N₅O₂Si, found 400.2 (MH+).

### 4-(4-ethoxy-1H-pyrrolo[2,3-b]pyridin-3-yl)-6-methylpyrimidin-2-amine

The title compound was synthesized in a manner similar to the deprotection route in Example 41, wherein 3-(2-aminopyrimidin-4-yl)-N,N-dimethyl-14(2-(trimethylsilyl)ethoxy)methyl)-1H-pyrazolo[3,4-d]pyrimidin-4-amine was substituted with 4-(4-ethoxy-14(2-(trimethylsilyl)ethoxy)methyl)-1H-pyrrolo[2,3-b]pyridin-3-yl)-6-methylpyrimidin-2-amine (26). The title compound was obtained in 7% yield after purification by preparative HPLC. ¹H-NMR (400 MHz, DMSO-d₆): δ 8.25 (d, 1H), 8.22 (s, 1H), 6.89 (d, 1H), 4.31 (q, 2H), 2.43 (s, 3H), 1.50 (t, 3H). MS (ES) for C₁₄H₁₈N₆O, found 270.1 (MH+).

### Example 39

### 3-(2-Aminopyrimidin-4-O-1H-pyrrolo[2,3-b]pyridin-5-amine

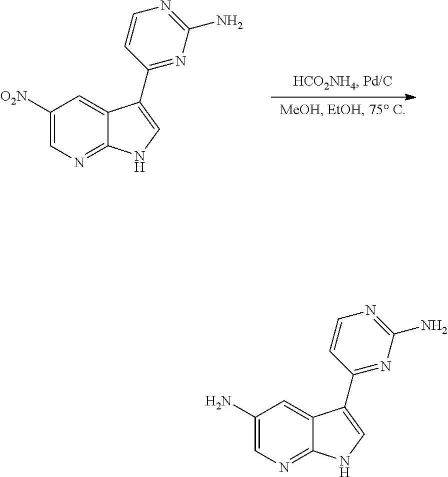

### 3-(2-Aminopyrimidin-4-yl)-1H-pyrrolo[2,3-b]pyridin-5-amine

A mixture of 4-(5-nitro-1H-pyrrolo[2,3-b]pyridin-3-yl)pyrimidin-2-amine (9.5 mg, 0.0371 mmol), HCO₂NH₄ (25.7 mg, 0.408 mmol), 10% Pd/C (24.2 mg, 0.0227 mmol), MeOH (1.0 ml) and EtOH (0.5 ml) was stirred at 75° C. for 3 h. After filtration, the filtrate was concentrated and the residue was purified by silica gel column chromatography (CH₂Cl₂/MeOH=9:1 with 1% Et₃N) to afford the title compound as a yellow powder (6.2 mg, 74%). ¹H NMR (400 MHz, DMSO-d₆) δ 11.7 (br s, 1H), 8.10 (d, 1H), 8.09 (d, 1H), 8.01 (d, 1H), 7.78 (d, 1H), 6.94 (d, 1H), 6.30 (s, 2H), 4.74 (br s, 2H). MS (ES) for C₁₁H₁₀N₆O, found 227.1 (MH+).

### 4-methoxy-1H-pyrrolo[2,3-b]pyridine 7-oxide (2)

To 4-methoxy-1H-pyrrolo[2,3-b]pyridine (1) (1.15 g, 7.78 mmol) in diethyl ether (70 mL) was added m-CPBA (2.01 g, 11.67 mmol) over 45 minutes and the reaction stirred at room temperature for an additional 2 hours. The solid was collected and washed with ether and recrystallized from acetone/ether to yield 4-methoxy-1H-pyrrolo[2,3-b]pyridine 7-oxide as a white solid (1.80 g, 141%, product contained 0.8 eqv. of m-CPMA). MS (EI) for C₈H₈N₂O₂, found 165.1 (MH+).

### (6-bromo-4-methoxy-1H-pyrrolo[2,3-b]pyridin-1-yl)(phenyl)methanone (3)

Solutions of benzoyl bromide (1.62 g, 8.75 mmol) in benzene (35 mL) and 1,1,1,3,3,3-hexamethyldisilazane (0.56 g, 3.51 mmol) in benzene (35 mL) were simultaneously added dropwise to a stirred solution of 4-methoxy-1H-pyrrolo[2,3-b]pyridine 7-oxide (2) in benzene (85 mL) over 2 hours under N₂. Upon completion of addition, the reaction was allowed to stir at room temperature for 1.5 hours. The mixture was washed twice with sat. NaHCO₃ brine, dried with Na₂SO₄, filtered and concentrated in vacuo. The resulting residue was purified by silica gel column chromatography with hexane/EtOAc (9:1) to give (6-bromo-4-methoxy-1H-pyrrolo[2,3-b]pyridin-1-yl)(phenyl)methanone (3) (482 mg, 70%, taken into account (2) contained m-CPBA). MS (EI) for C₁₆H₁₁BrN₂O₂, found 331.0 (MH+).

### 6-bromo-4-methoxy-1H-pyrrolo[2,3-b]pyridine (4)

To (6-bromo-4-methoxy-1H-pyrrolo[2,3-b]pyridin-1-yl)(phenyl)methanone (3) (400 mg, 1.21 mmol) in MeOH (35 mL) was added 1N NaOH (12 mL). The mixture was stirred at room temperature for 2 hours. Methanol was removed in vacuo and the residue was extracted with CHCl₃ (×2), washed with brine, dried with Na₂SO₄, filtered and concentrated in vacuo to yield the title compound (265 mg, 96%). ¹H NMR (400 MHz, DMSO-d₆) δ 11.82 (s, 1H), 7.30 (t, 1H), 6.82 (s, 1H), 6.43 (t, 1H), 3.96 (s, 3H). MS (EI) for C₈H₇BrN₂O, found 227.0 (MH+).

### 6-bromo-4-methoxy-1-((2-(trimethylsilyl)ethoxy)methyl)-1H-pyrrolo[2,3-b]pyridine (5)

Sodium hydride (60% dispersion, 22 mg, 0.92 mmol) was added to a cooled (0° C.) solution of 6-bromo-4-methoxy-1H-pyrrolo[2,3-b]pyridine (125 mg, 0.55 mmol) in THF, (5 mL). The reaction mixture was stirred at 0° C. for 15 minutes prior to the addition of 2-(trimethylsilyl)ethoxymethyl chloride (110 mg, 0.66 mmol). The mixture was then stirred at room temperature for 16 hours and then diluted with EtOAc. The mixture was washed with water and brine; dried with Na₂SO₄; filtered; and concentrated in vacuo to afford the title compound (203 mg, 103%). MS (EI) for C₁₄H₂₁BrN₂O₂Si, found 357.1 (MH+).

### 2-(dimethylamino)-N-(3-(4-methoxy-1-((2-(trimethylsilyl)ethoxy)methyl)-1H-pyrrolo[2,3-b]pyridin-6-Ylamino)propyl)acetamide (7)

In a sealed tube inserted with N₂, 6-bromo-4-methoxy-14(2-(trimethylsilyl)ethoxy)methyl)-1H-pyrrolo[2,3-b]pyridine (5) (200 mg, 0.56 mmol), N-(3-aminopropyl)-2-(dimethylamino)acetamide hydrochloride (6) (328 mg, 1.68 mmol), Pd₂(dba)₃ (15 mg, 0.016 mmol), rac-BINAP (35 mg, 0.056 mmol), Cs₂CO₃ (912 mg, 2.80 mmol), and toluene (8 mL) were combined and heated to 130° C. for 24 hours. The reaction mixture was cooled to room temperature; diluted with EtOAc; washed with water and brine; dried with Na₂SO₄; filtered; and concentrated in vacuo. The crude product was purified by silica gel column chromatography (5% MeOH in EtOAc) to give 2-(dimethylamino)-N-(3-(4-methoxy-1-((2-(trimethylsilyl)ethoxy)methyl)-1H-pyrrolo[2,3-b]pyridin-6-ylamino)propyl)acetamide (7) (64 mg, 26%). MS (EI) for C₂₁H₃₇N₅O₃Si, found 436.3 (MH+).

### 2-(dimethylamino)-N-(3-(4-methoxy-1H-pyrrolo[2,3-b]pyridin-6-ylamino)propyl)acetamide

2-(dimethylamino)-N-(3-(4-methoxy-1-((2-(trimethylsilyl)ethoxy)methyl)-1H-pyrrolo[2,3-b]pyridin-6-ylamino)propyl)acetamide (7) (63 mg, 0.14 mmol) was stirred in anhydrous THF (3 mL) followed by addition of tetrabutylammonium fluoride trihydrate (204 mg, 0.65 mmol). The mixture was heated to 75° C. under N₂ for 16 hours then evaporated to dryness. The crude residue was purified by prep HPLC to afford the title compound (16.8 mg, 39%). ¹H NMR (400 MHz, d₆-CDCl₃) δ 10.47 (s, 1H), 8.21 (s, 1H), 6.83 (m, 1H), 6.38 (m, 1H), 5.66 (s, 1H), 3.96 (s, 3H), 3.41 (m, 4H), 3.06 (s, 2H), 2.36 (s, 6H), 1.82 (m, 2H). MS (EI) for C₁₅H₂₃N₅O₂, found 306.2 (MH+).

### Representative Conditions under Scheme 14

### Example 40

### 4-methyl-6-(4-(4-methylpiperazin-1-yl)-1H-pyrrolo[2,3-b]pyridin-3-yl)pyrimidin-2-amine

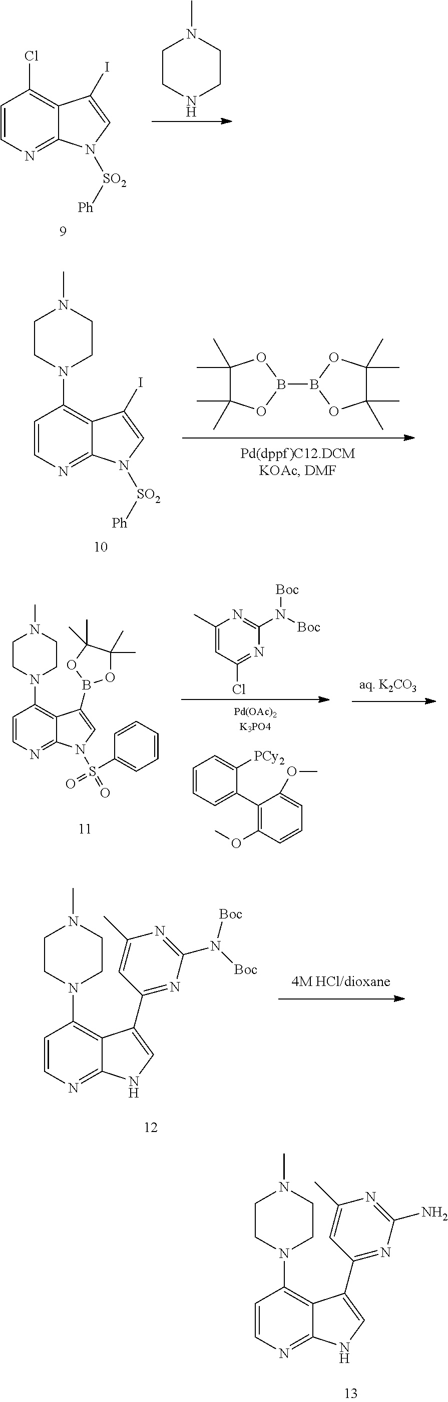

### 3-iodo-4-(4-methylpiperazin-1-yl)-1-(phenylsulfonyl)-1H-pyrrolo[2,3-b]pyridine (10)

4-chloro-3-iodo-1-(phenylsulfonyl)-1H-pyrrolo[2,3-b]pyridine (9) (1.0 g, 2.39 mmol) and N-methylpiperazine (7 mL) was stirred 110° C. in a sealed tube for 3.5 hours. The mixture was diluted with EtOAc; washed with water and brine; dried with Na₂SO₄; filtered and concentrated in vacuo. The crude product was purified by silica gel column chromatography (5% MeOH in EtOAc) to afford the title compound (298 mg, 26%). ¹H NMR (400 MHz, d₆-DMSO) δ 8.19 (d, 1H), 8.13 (d, 1H), 8.01 (s, 1H), 7.74 (t, 1H), 7.63 (t, 1H), 6.87 (d, 1H), 3.09 (br s, 4H), 2.59 (br s, 4H), 2.25 (s, 3H). MS (EI) for C₁₈H₁₉1N₄O₂S, found 483.1 (MH+).

### 4-(4-methylpiperazin-1-yl)-1-(phenylsulfonyl)-3-(4,4,5,5-tetramethyl-1,3,2-dioxaborolan-2-yl)-1H-pyrrolo[2,3-b]pyridine (11)

In a sealed tube inserted with N₂, 3-iodo-4-(4-methylpiperazin-1-yl)-1-(phenylsulfonyl)-1H-pyrrolo[2,3-b]pyridine (10) (178 mg, 0.37 mmol), Bis(pinacolato)-diboron (187 mg, 0.74 mmol), acetic acid potassium salt (109 mg, 1.11 mmol), Pd(dppf)Cl₂.CH₂Cl₂ (3 mg, 0.004 mmol), and DMF (2 mL) were combined and stirred at 100° C. for 16 hours. The reaction mixture was cooled to room temperature; diluted with EtOAc; washed with sat. NaHCO₃ and brine, dried with Na₂SO₄; filtered; and concentrated in vacuo to give 4-(4-methylpiperazin-1-yl)-1-(phenylsulfonyl)-3-(4,4,5,5-tetramethyl-1,3,2-dioxaborolan-2-yl)-1H-pyrrolo[2,3-b]pyridine (11) (233 mg, 130%). MS (EI) for C₂₄H₃₁BN₄O₄S, found 483.3 (MH+).

### Di-tert-butyl (4-methyl-6-(4-(4-methyl piperazin-14)-1H-pyrrolo[2,3-b]pyridin-3-yl)pyrimidin-2-yl)carbamate

In a sealed tube inserted with N₂, 4-(4-methylpiperazin-1-yl)-1-(phenylsulfonyl)-3-(4,4,5,5-tetramethyl-1,3,2-dioxaborolan-2-yl)-1H-pyrrolo[2,3-b]pyridine (11) (233 mg, 0.48 mmol), di-tert-butyl 4-chloro-6-methylpyrimidin-2-ylcarbamate (138 mg, 0.40 mmol), Pd(OAc)₂ (5 mg, 0.008 mmol), K₃PO₄ (171 mg, 0.81 mmol), dicyclohexyl(2′,6′-dimethoxybiphenyl-2-yl)phosphine (7 mg, 0.017 mmol) and n-BuOH (3 mL) were combined and heated to 100° C. for 1 hour. Subsequently, a solution of aq. saturated K₂CO₃ (1.5 mL) was added to the reaction and continued to heat at 100° C. for 2 hours. The reaction mixture was cooled to room temperature; diluted with EtOAc; washed with water and brine; dried with Na₂SO₄; and then filtered and concentrated in vacuo. The crude product was purified by silica gel column chromatography (5% MeOH, 20% Hexanes, 75% DCM) to afford the title compound (20 mg, 10%). MS (EI) for C₂₇H₃₇N₇O₄, found 524.3 (MH+).

### 4-methyl-6-(4-(4-methylpiperazin-1-yl)-1H-pyrrolo[2,3-b]pyridin-3-yl)pyrimidin-2-amine

Di-tert-butyl (4-methyl-6-(4-(4-methyl piperazin-1-yl)-1H-pyrrolo[2,3-b]pyridin-3-yl)pyrimidin-2-yl)carbamate (20 mg, 0.04 mmol) was stirred in 4M HCl/dioxane (3 mL) at room temperature for 2 hours then subsequently warmed to 50° C. for 1 hour. The solvent was removed and the residue was triturated with acetonitrile to afford 4-methyl-6-(4-(4-methylpiperazin-1-yl)-1H-pyrrolo[2,3-b]pyridin-3-yl)pyrimidin-2-amine as a solid (6.5 mg, 50%). ¹H NMR (400 MHz, DMSO-d₆) δ 12.93 (s, 1H), 9.33 (s, 1H), 8.33 (d, 2H), 7.30 (s, 1H), 7.19 (d, 1H), 3.95 (m, 2H), 3.43 (m, 4H), 3.06 (m, 2H), 2.82 (s, 3H), 2.51 (s, 3H). MS (EI) for C₁₇H₂₁N₇, found 324.2 (MH+).

### Representative Conditions under Scheme 15

### Example 41

### 4-(4-Methoxy-1H-pyrrolo[2,3-b]pyridin-2-yl)pyrimidin-2-amine

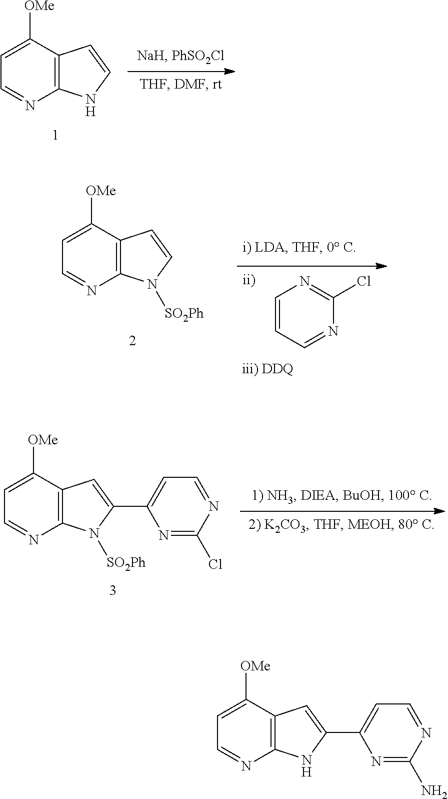

### 4-Methoxy-1-(phenylsulfonyl)-1H-pyrrolo[2,3-b]pyridine

To a stirred solution of 4-methoxy-1H-pyrrolo[2,3-b]pyridine 1 (500 mg, 3.37 mmol), DMF (0.6 mL) and THF (6 mL) at 0° C. was added NaH (290 mg, 60% dispersion in mineral oil, 4.39 mmol). After stirring for 10 min at room temperature, benzenesulfonyl chloride (0.69 mL, 5.39 mmol) was added. After stirring for 1 h, aqueous saturated NH₄Cl solution (10 mL) was added and the resulting mixture was extracted with EtOAc (3×5 mL). The combined organic layers were concentrated and the resulting material was purified by silica gel column chromatography (Hexane/EtOAc/CH₂Cl₂=4:1:1→4:1:2) to afford 4-Methoxy-1-(phenylsulfonyl)-1H-pyrrolo[2,3-b]pyridine 2 (810 mg, 83%) as a white powder. MS (EI) for C₁₄H₁₂N₂O₃S, found 289.1 (MH+).

### 2-(2-Chloropyrimidin-4-yl)-4-methoxy-1-(phenylsulfonyl)-1H-pyrrolo[2,3-b]pyridine

To a stirred solution of 4-methoxy-1-(phenylsulfonyl)-1H-pyrrolo[2,3-b]pyridine 2 (300 mg, 1.04 mmol) in THF (5 mL) at 0° C. was added LDA (0.73 mL, 2M in heptane/THF/ethylbenzene, 1.46 mmol). After stirring for 30 min, the resulting mixture was added to the stirred solution of 2-chloropyrimidine (143 mg, 1.23 mmol) in THF (2 mL) at 0° C. After stirring for another 30 min, H₂O (0.056 mL, 3.12 mmol) was added and stirring was continued for 10 min. DDQ (472 mg, 2.08 mmol) was added and the resulting mixture was maintained at room temperature for 30 min. Hexane (3 mL) was added followed by aqueous NaOH solution (3 mL, 3N). The resulting mixture was stirred for 5 min, water (30 mL) was added and then the layers were separated. The aqueous phase was extracted with EtOAc (4×5 mL), and the combined organic layers were concentrated and the resulting material was purified by silica gel chromatography (Hexane/EtOAc/CH₂Cl₂=4:1:1→2:1:1) to afford 2-(2-chloropyrimidin-4-yl)-4-methoxy-1-(phenylsulfonyl)-1H-pyrrolo[2,3-b]pyridine 3 (205 mg, 49%) as a pale pink powder. MS (EI) for C₁₈H₁₃ClN₄O₃S, found 401.0 (MH+).

### 4-(4-Methoxy-1H-pyrrolo[2,3-b]pyridin-2-yl)pyrimidin-2-amine

A mixture of 2-(2-chloropyrimidin-4-yl)-4-methoxy-1-(phenylsulfonyl)-1H-pyrrolo[2,3-b]pyridine 3 (45 mg, 0.112 mmol), NH₃ (0.84 mL, 2M in MeOH, 1.68 mmol), DIEA (0.040 mL, 0.224 mmol) and BuOH (1 mL) was stirred at 120° C. for 10 h. After removing volatile materials in vacuo, K₂CO₃ (61.9 mg, 0.448 mmol), THF (1 mL) and MeOH (1 mL) were added and the resulting mixture was stirred at 80° C. for 2 h. After cooling to room temperature, the mixture was concentrated in vacuo. The residue was treated with CH₂Cl₂ (3 mL) and aqueous, saturated NaHCO₃ (3 mL), and the layers were separated. The aqueous phase was extracted with CH₂Cl₂ (5×1 mL) and the combined organic layers were concentrated. The resulting material was purified by preparative HPLC to give the title compound (7.5 mg, 28%, 2 steps) as a white powder. ¹H NMR (400 MHz, DMSO-d₆) δ 12.1 (s, 1H), 8.27 (d, J=5.2 Hz, 1H), 8.17 (d, J=5.6 Hz, 1H), 7.17 (d, J=5.2 Hz, 1H), 7.17 (s, 1H), 6.67 (d, J=6.0 Hz, 1H), 6.54 (s, 2H), 3.96 (s, 3H). MS (EI) for C₁₂H₁₁N₆O, found 242.1 (MH+).

### Example 42

### 3-(1H-Indazol-6-yl)-1H-pyrazolo[3,4-d]pyrimidin-4-ol

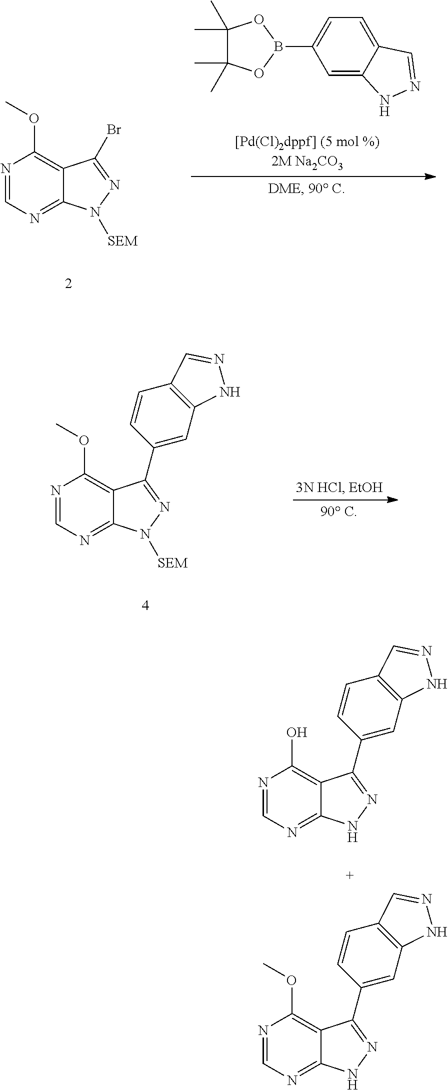

### 3-(1H-Indazol-6-yl)-4-methoxy-1-[[2-(trimethylsilyl)ethoxy]methyl]-1H-pyrazolo[3,4-d]pyrimidine

The title compound was synthesized in a manner similar to Example 22, wherein 5-(4,4,5,5-tetramethyl-1,3,2-dioxaborolan-2-yl)pyridin-2-amine was substituted with 6(4,4,5,5-tetramethyl-1,3,2-dioxaborolan-2-yl)-1H-indazole. After purification by flash chromatography (91:8:1 dichloromethane/methanol/28% (w/w) ammonium hydroxide), 3-(1H-Indazol-6-yl)-4-methoxy-1-[[2-(trimethylsilyl)ethoxy]methyl]-1H-pyrazolo[3,4-d]pyrimidine 4 was obtained as a solid (39 mg, 54% yield). MS (EI) for C₁₉H₂₄N₆O₂Si, found 397 (MH⁺).

### 3-(1H-Indazol-64)-1H-pyrazolo[3,4-d]pyrimidin-4-ol

A solution of 3-(1H-indazol-6-yl)-4-methoxy-1-[[2-(trimethylsilyl)ethoxy]methyl]-1H-pyrazolo[3,4-d]pyrimidine 4 (39 mg, 0.098 mmol) in absolute ethanol (5 mL) and 3 N hydrochloric acid (2 mL) was heated to a gentle reflux for 16 h. After cooling to room temperature, the mixture was concentrated and purified by preparative HPLC (reverse-phase, 0.1% TFA in acetonitrile/0.05% TFA in water). 3-(1H-Indazol-6-yl)-1H-pyrazolo[3,4-c]pyrimidin-4-ol was obtained as a solid (6 mg, 24% yield) ¹H-NMR (400 MHz, DMSO-d6): δ 13.9 (br s, 1H), 13.3 (br s, 1H), 12.2 (m, 1H), 8.90 (s, 1H), 8.06 (m, 3H), 7.79 (m, 1H). MS (EI) for C₁₂H₈N₆O, found 253 (MH⁺).

### Example 43

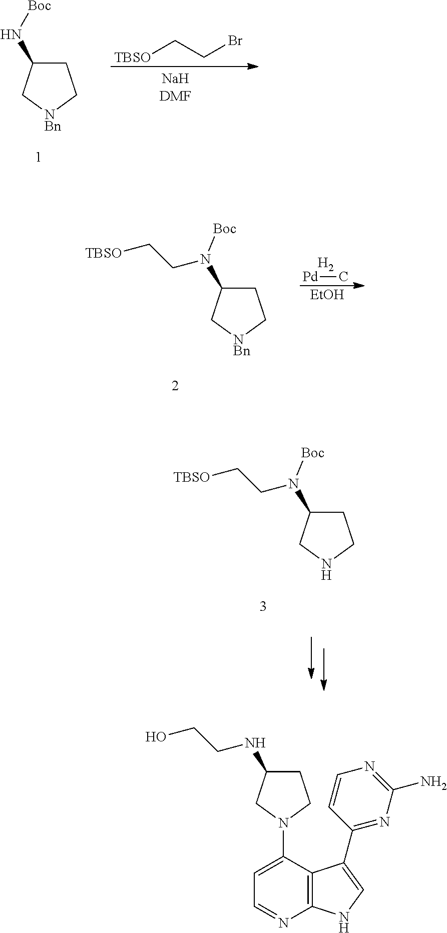

### (S)-tert-Butyl 1-benzylpyrrolidin-3-yl(2-(tert-butyldimethylsilyloxy)ethyl)carbamate (2)

To a cooled (0° C.) solution of (S)-tert-butyl 1-benzylpyrrolidin-3-ylcarbamate (1.0 g, 3.62 mmol) in DMF was added NaH (60% dispersion, 175 mg, 4.34 mmol). The resulting suspension was stirred at 0° C. for 15 minutes. 2-(bromoethoxy)-(tert-butyl)dimethylsilane (0.78 mL, 4.34 mmol) was added dropwise and the reaction mixture was allowed to warm to room temperature. After stirring at room temperature for 18 hours, the reaction was quenched by addition of sat. NH₄Cl and extracted three times with ethyl acetate. The combined organic extracts were washed with brine, dried (Na₂SO₄), filtered and concentrated to a yellow paste. The crude product was purified by silica gel chromatography to give the title compound as a yellow oil (1.07 g, 68%). MS (ES) for C₂₄H₄₂N₂O₃Si, found 435 (MH⁺).

### (S)-tert-Butyl 2-(tert-butyldimethylsilyloxy)ethyl(pyrrolidin-3-yl)carbamate (3)

A solution of 2 in ethanol was stirred under a hydrogen atmosphere for 15 hour at room temperature and for 2 hours at 55° C. The resulting mixture was filtered through celite and concentrated to give the title compound (0.81 g, 96%). ¹H-NMR (400 MHz, CDCl₃): δ 5.92 (s, br, 2H), 4.23 (s, br, 1H), 3.63-3.80 (m, 2H), 3.17-3.51 (m, 4H), 2.98-3.11 (m, 1H), 2.09-2.31 (s, br, 1H), 1.99-2.10 (m, 1H), 1.45 (s, 9H), 0.89 (s, 9H), 0.05 (s, 6H). MS (ES) for C₁₇H₃₆N₂O₃Si, found 345 (MH⁺).

2-({(3S)-1-[3-(2-Aminopyrimidin-4-O-1H-pyrrolo[2,3-b]pyridin-4-yl]pyrrolidin-3-yl}amino)ethanol ¹H-NMR (400 MHz, MeOH-d₄): δ 8.22 (1H, d), 8.04 (1H, d), 7.70 (1H, s), 6.96 (1H, d), 6.63 (1H, d), 3.59-3.75 (m, 4H), 3.31-3.37 (m, 1H), 3.13-3.23 (m, 2H), 2.88-2.98 (m, 2H), 2.20-2.31 (m, 1H), 1.79-1.90 (m, 1H). MS (ES) for C₁₇H₂₃N₇O, found 340.2 (MH⁺).

Using analogous synthetic techniques as in Example 43, and substituting with the appropriate alternative starting materials, the following compounds were prepared. Alternative starting materials were obtained commercially unless otherwise indicated.

### N-[3-(2-aminopyrimidin-4-yl)-1H-pyrrolo[2,3-b]pyridin-4-yl]-N-methylglycine

¹H-NMR (400 MHz, DMSO-d₆): δ 8.12 (m, 1H), 7.94 (m, 1H), 7.66 (s, 1H), 6.97 (m, 1H), 6.52 (m, 1H), 6.37 (s, 2H), 3.50 (s, 2H), 2.83 (s, 3H). MS (ES) for C₁₄H₁₄N₆O₂, found 299.1 (MH+).

### 4-{4-[3-(aminomethyl)pyrrolidin-1-yl]-1H-pyrrolo[2,3-b]pyridin-3-yl}pyrimidin-2-amine

¹H-NMR (400 MHz, DMSO-d₆): δ 8.13 (d, 1H), 7.94 (d, 1H), 7.56 (s, 1H), 6.75 (d, 1H), 6.44 (d, 1H), 6.40 (s, 2H), 3.25 (m, 2H), 3.08 (m, 1H), 3.00 (m, 1H), 2.889 (m, 1H), 2.61 (m, 2H), 2.27 (m, 1H), 1.46 (m, 1H). MS (ES) for C₁₆H₁₉N₇, found 310.2 (MH+).

### 3-(2-aminopyrimidin-4-yl)-N-[(1-methyl-1H-imidazol-2-yl)methyl]-1H-pyrrolo[2,3-b]pyridin-4-amine

¹H-NMR (400 MHz, DMSO-d₆): δ 10.44 (m, 1H), 8.16 (s, 1H), 8.04 (d, 1H), 7.88 (d, 1H), 7.17 (m, 1H), 7.09 (d, 1H), 6.83 (m, 1H), 6.37 (d, 1H), 4.51 (d, 2H), 3.66 (s, 3H). MS (ES) for C₁₆H₁₆N₁₈, found 321.1 (MH+).

### 3-(2-Aminopyrimidin-4-yl)-N-cyclopentyl-1H-pyrrolo[2,3-b]pyridin-4-amine

¹H-NMR (400 MHz, MeOH-d₄): δ 8.23 (d, 1H), 8.12 (s, 1H), 7.92 (d, 1H), 7.24 (d, 1H), 6.68 (d, 1H), 4.12-4.20 (m, 1H), 2.20-2.32 (m, 2H), 1.70-1.90 (m, 6H). MS (ES) for C₁₆H₁₈N₆, found 295.2 (MH⁺).

### Example 44

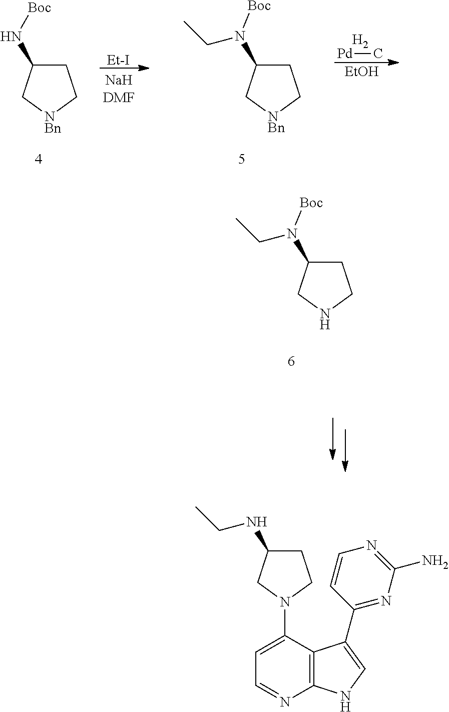

### (S)-tert-Butyl ethyl(pyrrolidin-3-yl)carbamate (6)

The title compound was synthesized in a manner similar to compound 3, wherein 2-(bromoethoxy)-(tert-butyl)dimethylsilane was substituted with ethyl iodide. ¹H-NMR (400 MHz, CDCl₃): δ 5.32 (s, br, 1H), 4.18-4.29 (m, 1H), 3.27-3.45 (m, 2H), 3.22 (q, 2H), 3.01-3.16 (m, 2H), 2.14-2.28 (m, 1H), 1.46 (s, 9H), 1.11 (t, 2H). MS (ES) for C₁₁H₂₂N₂O₂, found 215 (MH⁺).

Using analogous synthetic techniques as in Example 43, and substituting with the appropriate alternative starting materials, the following compounds were prepared. Alternative 8 starting materials were obtained commercially unless otherwise indicated.

### 4-{4-[(3S)-3-(Ethylamino)pyrrolidin-1-yl]-1H-pyrrolo[2,3-b]pyridin-3-yl}pyrimidin-2-amine

¹H-NMR (400 MHz, MeOH-d₄): δ 8.23 (d, 1H), 8.05 (d, 1H), 7.73 (s, 1H), 6.96 (d, 1H), 6.65 (d, 1H), 3.73-3.81 (m, 1H), 3.63-3.71 (m, 1H), 3.16-3.26 (m, 2H), 2.90-3.05 (m, 2H), 2.26-2.36 (m, 1H), 1.27 (t, 3H). MS (ES) for C₁₇H₂₃N₇, found 324.2 (MH⁺).

### 3-(2-Aminopyrimidin-4-yl)-N-pyrrolidin-3-yl-1H-pyrrolo[2,3-b]pyridin-4-amine

¹H-NMR (400 MHz, MeOH-d₄): δ 8.45 (s, 1H), 8.25 (d, 1H), 8.13 (d, 1H), 7.49 (d, 1H), 6.83 (d, 1H), 4.67-4.76 (m, 1H), 3.78-3.86 (m, 1H), 3.55-3.65 (m, 1H), 3.41-3.50 (m, 1H), 2.65-2.76 (m, 1H), 2.31-2.43 (m, 1H). MS (ES) for C₁₅H₁₇N₇, found 296.2 (MH⁺).

### Example 45

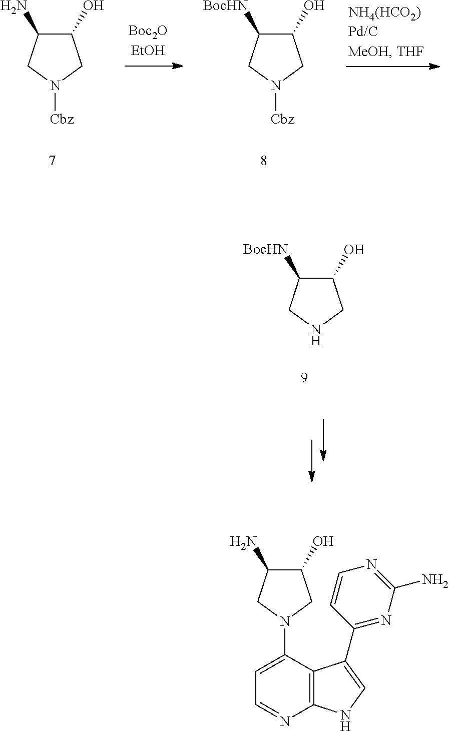

(3R,4R)-Benzyl 3-amino-4-hydroxypyrrolidine-1-carboxylate (7) was prepared enantioselectively following the known procedure as referenced in Jacobsen, E. N. et al J Org Lett 1997, 62, 4197.

### (3R,4R)-Benzyl 3-(tert-butoxycarbonylamino)-4-hydroxypyrrolidine-1-carboxylate (8)

To a cooled (0° C.) solution of 7 (670 mg, 2.84 mmol) in ethanol (10 mL) was added di-tert-butyl dicarbonate (618 mg, 2.84 mmol). The resulting solution was stirred at room temperature for 18 hours and concentrated under vacuum. The crude product was purified by flash chromatography to give the title compound as a white foam (0.93 g, 98%). ¹H-NMR (400 MHz, CDCl₃): δ 7.28-8.37 (m, 5H), 5.12 (s, 2H), 4.22 (s, br, 1H), 3.96 (s, br, 1H), 3.80-3.87 (m, 1H), 3.66-3.73 (m, 1H), 3.24-3.44 (m, 2H), 1.43 (s, 9H).

### tert-Butyl (3R,4R)-4-hydroxypyrrolidin-3-ylcarbamate (9)

To a cooled (0° C.) solution of 8 (0.49 g, 1.47 mmol) in methanol (3 mL) and THF (3 mL) was added Pd—C (10 wt %, 200 mg) and ammonium formate (300 mg, 4.76 mmol). The resulting solution was stirred at room temperature for 3.5 hours. The suspension was then filtered through celite and concentrated to give the title compound as a white solid (0.297 g, quant.). ¹H-NMR (400 MHz, MeOH-d₄): δ 8.51 (s, 1H), 4.27-4.31 (m, 1H), 3.97-4.02 (m, 1H), 3.57-3.64 (m, 1H), 3.38-3.44 (m, 1H), 3.27-3.29 (m, 1H), 3.22 (d, 1H), 1.44 (s, 9H).

Using analogous synthetic techniques as in Example 45, and substituting with the appropriate alternative starting materials, the following compounds were prepared. Alternative8 starting materials were obtained commercially unless otherwise indicated.

### (3R,4R)-4-Amino-1-[3-(2-aminopyrimidin-4-yl)-1H-pyrrolo[2,3-b]pyridin-4-yl]pyrrolidin-3-ol

¹H-NMR (400 MHz, MeOH-d₄): δ 8.26 (d, 1H), 8.10 (s, 1H), 8.05 (d, 1H), 7.31 (d, 1H), 6.84 (d, 1H), 4.41-4.48 (m, 1H), 4.16-4.22 (m, 1H), 3.61-3.79 (m, 4H). MS (ES) for C₁₅H₁₇N₇O, found 312.2 (MH⁺).

### 3-(2-Aminopyrimidin-4-yl)-N-piperidin-3-yl-1H-pyrrolo[2,3-b]pyridin-4-amine

¹H-NMR (400 MHz, MeOH-d₄): δ 8.10 (s, 1H), 7.79 (d, 1H), 7.84 (d, 1H), 7.21 (d, 1H), 6.55 (d, 1H), 3.99-4.09 (m, 1H), 3.46-3.52 (m, 1H), 3.20-3.27 (m, 1H), 2.87-3.02 (m, 2H), 2.15-2.27 (m, 1H), 1.87-1.99 (m, 1H), 1.70-1.84 (m, 2H). MS (ES) for C₁₆H₁₉N₇, found 310.2 (MH⁺).

### Example 46

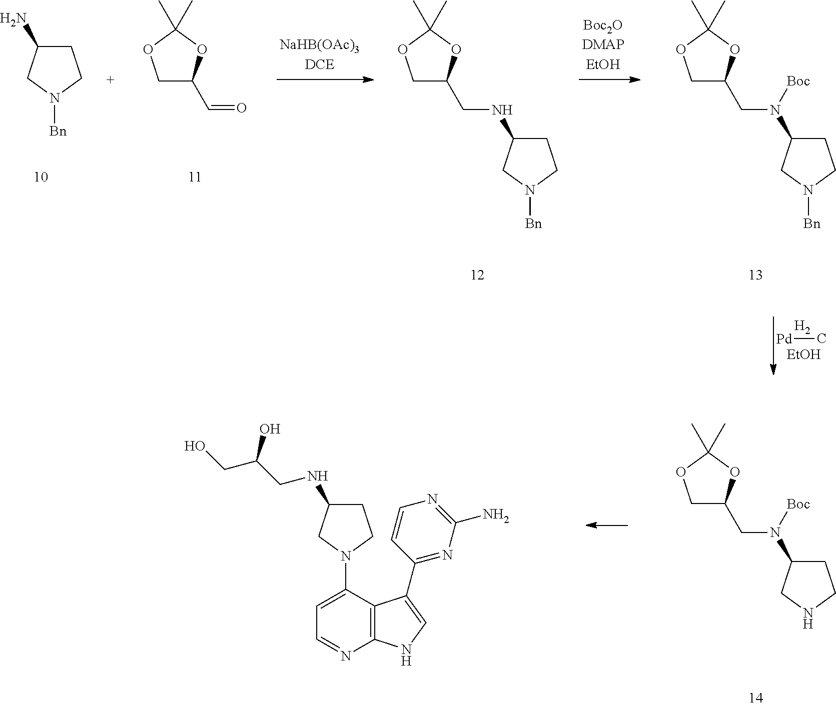

### (S)-1-Benzyl-N-(((S)-2,2-dimethyl-1,3-dioxolan-4-yl)methyl)pyrrolidin-3-amine (12)

To a solution of 10 (2.5 g, 14 mmol) in DCE (55 mL) was added 11 (1.66 g, 12.8 mmol). The resulting mixture was stirred at room temperature for 5 minutes before the addition of sodium tri(acetoxy)boronhydride (4.2 g, 20 mmol). The suspension was then stirred at room temperature for 3 hours. The reaction was quenched with sat. NaHCO₃ and extracted three times with DCM. The combined organic extracts were washed with water, dried (Na₂SO₄), filtered and concentrated to give the title compound 12 (2.14 g, 58%). MS (ES) for C₁₇H₂₆N₂O₂, found 291 (MH⁺).

### tert-Butyl(S)-1-benzylpyrrolidin-3-yl(((S)-2,2-dimethyl-1,3-dioxolan-44)methyl)carbamate (13)

A mixture of 12 (2.14 g, 7.38 mmol), di-tert-butyl dicarbonate (3.2 g, 15 mmol) and DMAP (20 mg, 0.16 mmol) in ethanol was stirred at room temperature for 2 days. The resulting mixture was then evaporated to dryness and purified by flash chromatography to give the title compound (1.97 g, 68%). MS (ES) for C₂₂H₃₄N₂O₄, found 391 (MH⁺).

### tert-Butyl((S)-2,2-dimethyl-1,3-dioxolan-4-yl)methyl((S)-pyrrolidin-3-yl)carbamate (14)

A suspension of 13 (1.97 g, 5.04 mmol) and palladium on carbon (10 wt %, 200 mg) in ethanol (50 mL) was stirred under a hydrogen atmosphere for 18 hours. The resulting mixture was then filtered through celite and concentrated to give the title compound as a colorless paste (1.64 g, quant.)¹H-NMR (400 MHz, CDCl₃): δ 4.10-4.33 (m, 2H), 4.02-4.08 (m, 1H), 3.64 (t, 1H), 3.02-3.25 (m, 3H), 2.76-2.93 (m, 2H), 1.86-2.09 (m, 3H), 1.46 (s, 9H), 1.42 (s, 3H), 1.39 (s, 3H). MS (ES) for C₁₅H₂₈N₂O₄, found 301.2 (MH⁺).

Using analogous synthetic techniques as in Example 46, and substituting with the appropriate alternative starting materials, the following compounds were prepared. Alternative starting materials were obtained commercially unless otherwise indicated.

### (2S)-3-({(3S)-1-[3-(2-Aminopyrimidin-4-yl)-1H-pyrrolo[2,3-b]pyridin-4-yl]pyrrolidin-3-yl}amino)propane-1,2-diol

¹H-NMR (400 MHz, MeOH-d₄): δ 8.25 (d, 1H), 8.10 (s, 1H), 8.05 (d, 1H), 7.30 (d, 1H), 6.86 (d, 1H), 4.10-4.17 (m, 1H), 3.86-4.03 (m, 3H), 3.71-3.85 (m, 2H), 3.50-3.63 (m, 5H), 3.24-3.67 (m, 21H), 3.98-3.05 (m, 1H), 2.45-2.55 (m, 1H), 2.18-2.30 (m, 1H). MS (ES) for C₁₈H₂₃N₇O₂, found 370.2 (MH⁺).

### 3-(2-Aminopyrimidin-4-yl)-N-phenyl-1H-pyrrolo[2,3-b]pyridin-4-amine

¹H-NMR (400 MHz, MeOH-d₄): δ 8.13 (d, 1H), 8.02 (s, 1H), 7.88 (d, 1H), 7.37-7.47 (m, 4H), 7.09-7.16 (m, 2H), 6.85 (d, 1H). MS (ES) for C₁₇H₁₄N₆, found 303.2 (MH⁺).

### 3-(2-Aminopyrimidin-4-yl)-N-(1-methylpyrrolidin-3-O-1H-pyrrolo[2,3-b]pyridin-4-amine

¹H-NMR (400 MHz, MeOH-d₄): δ 8.07 (d, 1H), 7.94 (s, 1H), 7.84 (d, 1H), 7.06 (d, 1H), 6.30 (d, 1H), 4.29-4.35 (m, 1H), 3.23-3.29 (m, 1H), 3.09-3.15 (m, 1H), 2.67-2.75 (m, 1H), 2.39-2.57 (m, 6H), MS (ES) for C₁₆H₂₀N₇, found 310.2 (MH⁺).

### 3-(2-Aminopyrimidin-4-yl)-N-methyl-N-phenyl-1H-pyrrolo[2,3-b]pyridin-4-amine

¹H-NMR (400 MHz, MeOH-d₄): δ 8.20 (d, 1H), 7.89 (d, 1H), 7.63 (s, 1H), 6.99-7.05 (m, 3H), 6.69-6.78 (m, 2H), 6.57 (d, 1H), 3.35 (s, 3H). MS (ES) for C₁₈H₁₆N₆, found 317.2 (MH⁺).

### {3-amino-1-[3-(2-aminopyrimidin-4-yl)-1H-pyrrolo[2,3-b]pyridin-4-yl]pyrrolidin-3-yl}methanol

¹H-NMR (400 MHz, MeOH-d₄): δ 8.26 (d, 1H), 8.04 (d, 1H), 7.83 (s, 1H), 7.07 (d, 1H), 6.76 (d, 1H), 3.79 (d, 1H), 3.60-3.68 (m, 4H), 3.56 (d, 1H), 2.06-2.59 (m, 2H). MS (ES) for C₁₆H₁₉N₇O, found 326.2 (MH⁺).

### (3S,4S)-1-[3-(2-Aminopyrimidin-4-yl)-1H-pyrrolo[2,3-b]pyridin-4-yl]pyrrolidine-3,4-diol

¹H-NMR (400 MHz, MeOH-d₄): δ 8.17 (d, 1H), 7.94 (d, 1H), 7.59 (s, 1H), 6.92 (d, 1H), 6.57 (d, 1H), 4.04 (m, 2H), 3.66 (m, 2H), 3.20 (m, 2H), MS (ES) for C₁₅H₁₇N₆O₂, found 313.2 (MH⁺).

### 1-[3-(2-Aminopyrimidin-4-yl)-1H-pyrrolo[2,3-b]pyridin-4-yl]azetidin-3-ol

¹H-NMR (400 MHz, MeOH-d₄): δ 8.22 (d, 1H), 7.98 (d, 1H), 7.56 (s, 1H), 6.93 (d, 1H), 6.32 (d, 1H), 4.46-4.54 (m, 1H), 4.02-4.08 (m, 2H), 3.61-3.67 (m, 2H). MS (ES) for C₁₄H₁₅N₆O₂, found 283.2 (MH⁺).

### Example 47

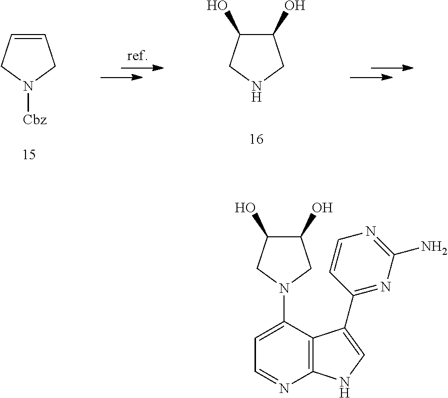

cis-pyrrolidine-3,4-diol (16): Compound 16 was prepared according to procedure described in J. Med. Chem. 1990, 7, 1962.

### (3R,4S)-1-[3-(2-aminopyrimidin-4-yl)-1H-pyrrolo[2,3-b]pyridin-4-yl]pyrrolidine-3,4-diol

¹H-NMR (400 MHz, MeOH-d₄): δ 8.17 (d, 1H), 7.93 (d, 1H), 7.56 (s, 1H), 6.87 (d, 1H), 6.51 (d, 1H), 4.09-4.14 (m, 2H), 3.44-3.52 (m, 2H), 3.22-3.28 (m, 2H). MS (ES) for C₁₅H₁₆N₆O₂, found 313.2 (MH⁺).

### Example 48

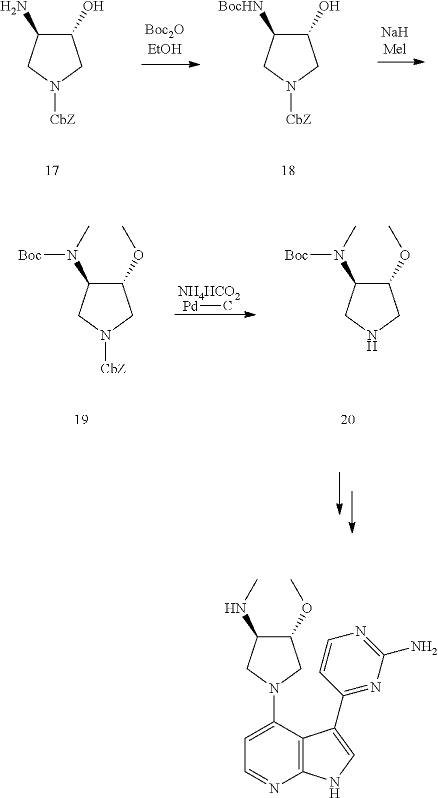

### (3R,4R)-Benzyl 3-(tert-butoxycarbonylamino)-4-hydroxypyrrolidine-1-carboxylate (18)

A mixture of 17 (670 mg. 2.84 mmol) and di-tert-butyl dicarbonate (618 mg, 2.84) in ethanol (10 mL) was stirred at room temperature for 18 hours. The mixture was evaporated to dryness. The crude product was purified by flash chromatography to give the title compound as a white foam (931 mg, 98%). ¹H-NMR (400 MHz, CDCl₃): δ 7.27-7.38 (m, 5H), 5.12 (s, 2H), 4.18-4.27 (s, br, 1H), 3.89-4.01 (m, 1H), 3.79-3.88 (m, 1H), 3.66-3.74 (m, 1H), 3.23-3.43 (m, 4H).

### (3R,4R)-Benzyl 3-(tert-butoxycarbonyl(methyl)amino)-4-methoxypyrrolidine-1-carboxylate (19)

To a cooled (0° C.) solution of 18 (460 mg, 2.28 mmol) in THF (10 mL) was added NaH (60% dispersion, 180 mg, 4.56 mmol) and methyl iodide (0.5 mL). After stirring at room temperature for 1 hour, the reaction was quenched by the addition of sat. aqueous NH₄Cl. The mixture was then extracted with ethyl acetate. The combined extracts were washed with brine, dried (Na₂SO₄), filtered and concentrated. The crude product was purified by flash chromatography to give the title compound as a clear colorless paste (0.51 g, 61%). ¹H-NMR (400 MHz, CDCl₃): δ 7.29-7.41 (m, 5H), 5.14 (s, 2H), 3.83-3.91 (m, 2H), 3.64-3.77 (m, 2H), 3.34-3.50 (m, 2H), 3.39 (s, 3H), 2.78 (s, 3H), 1.47 (s, 9H).

### tert-Butyl (3R,4R)-4-methoxypyrrolidin-3-yl(methyl)carbamate (20)

A mixture of 19 (500 mg, 1.37 mmol), ammonium formate (500 mg) and palladium on carbon (10 wt %, 100 mg) in methanol (10 mL) and THF (10 mL) was stirred at room temperature for 15 hours. The reaction mixture was the filtered through celite and concentrated to give the title compound as a white solid (427 mg, quant.). MS (ES) for C₁₁H₂₂N₂O₃, found 231 (MH⁺).

### 4-{4-[(3R,4R)-3-(Methylamino)-4-(methyloxy)pyrrolidin-1-yl]-1H-pyrrolo[2,3-b]pyridin-3-yl}pyrimidin-2-amine

¹H-NMR (400 MHz, MeOH-d₄): δ 8.20 (d, 1H), 8.05 (d, 1H), 7.71 (s, 1H), 6.98 (d, 1H), 6.63 (d, 1H), 3.82-3.88 (m, 1H), 3.65-3.72 (m, 1H), 3.41-3.47 (m, 1H), 3.32-3.38 (m, 1H), 3.25 (s, 3H), 3.21-3.23 (m, 1H), 3.01-3.07 (m, 1H), 2.52 (s, 3H). MS (ES) for C₁₇H₂₃N₇O, found 340.2 (MH⁺).

### Example 49

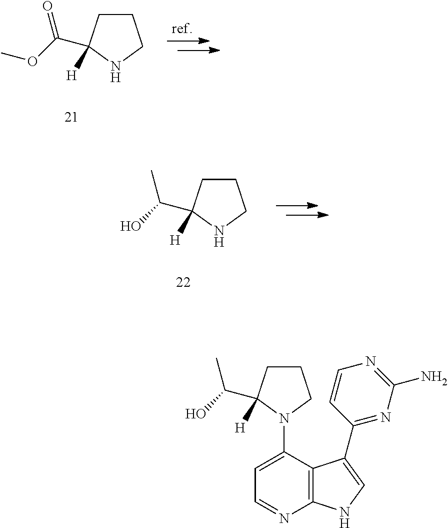

(R)-1-((S)-pyrrolidin-2-yl)ethanol (22): Compound 21 was prepared according to procedure described in J. Org. Chem. 2003, 25, 9747.

### (1R)-1-{(2S)-1-[3-(2-Aminopyrimidin-4-yl)-1H-pyrrolo[2,3-b]pyridin-4-yl]pyrrolidin-2-yl}ethanol

¹H-NMR (400 MHz, MeOH-d₄): δ 8.18 (d, 1H), 7.99 (d, 1H), 7.67 (s, 1H), 7.04 (d, 1H), 6.76 (d, 1H), 4.14-4.21 (m, 1H), 3.89-3.95 (td, 1H), 3.14-3.22 (m, 1H), 3.04-3.11 (m, 1H), 1.92-2.02 (m, 2H), 1.70-1.80 (m, 1H), 1.55-1.67 (m, 1H), 1.18 (d, 3H). MS (ES) for C₁₇H₂₁N₆O, found 325.2 (MH⁺).

### (3R,4R)-1-[3-(2-aminopyrimidin-4-yl)-1H-pyrrolo[2,3-b]pyridin-4-yl]pyrrolidine-3,4-diol

¹H-NMR (400 MHz, MeOH-d₄): δ 8.25 (d, 1H), 8.01 (d, 1H), 7.93 (s, 1H), 7.13 (d, 1H), 6.87 (d, 1H), 4.16 (d, 2H), 3.87 (dd, 2H), 3.51 (d, 2H). MS (ES) for C₁₅H₁₆N₆O₂, found 313.2 (MH⁺).

### Example 50

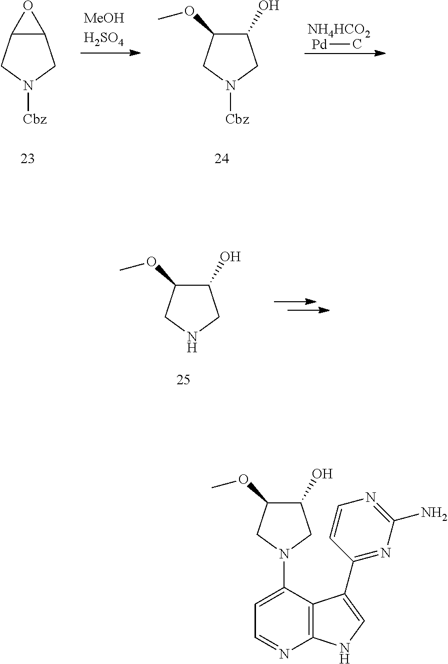

### trans-Benzyl 3-hydroxy-4-methoxypyrrolidine-1-carboxylate (24)

To a solution of 23 (850 mg, 3.88 mmol) in methanol was added dropwise conc. H₂SO₄ (0.1 mL). The reaction mixture was stirred at room temperature for 2 hours before being poured onto a 0.01N NaOH solution. The product was then extracted with ethyl acetate. The combined extracts were washed with sat. NaHCO₃ and brine, dried (Na₂SO₄), filtered and concentrated to give the title compound (1.02 g, quant.). ¹H-NMR (400 MHz, CDCl₃): δ 7.27-7.40 (m, 5H), 5.12 (s, 2H), 4.26 (s, br, 1H), 3.67-3.75 (m, 1H), 3.57-3.66 (m, 3H), 3.39-3.51 (m, 2H), 3.35 (s, 3H).

### trans-4-Methoxypyrrolidin-3-ol (25)

A mixture of 24 (1.02 g, 4.06 mmol), ammonium formate (1.5 g) and palladium on carbon (10 wt %, 300 mg) in methanol (15 mL) and THF (15 mL) was stirred at room temperature for 2.5 hours. The reaction mixture was the filtered through celite and concentrated to give the title compound as a clear colorless paste (475 mg, quant.) MS (ES) for C₅H₁₁NO₂ found 118 (MH⁺).

### (3R,4R)-1-[3-(2-Aminopyrimidin-4-yl)-1H-pyrrolo[2,3-b]pyridin-4-yl]-4-(methyloxy)pyrrolidin-3-ol

¹H-NMR (400 MHz, MeOH-d₄): δ 8.16 (d, 1H), 7.95 (d, 1H), 7.62 (s, 1H), 6.92 (d, 1H), 6.56 (d, 1H), 4.15-4.20 (m, 1H), 3.66-3.70 (m, 1H), 3.52-3.61 (m, 2H), 3.25 (dd, 1H), 3.14 (dd, 1H). MS (ES) for C₁₆H₁₉N₆O₂, found 327.2 (MH⁺).

### Example 51

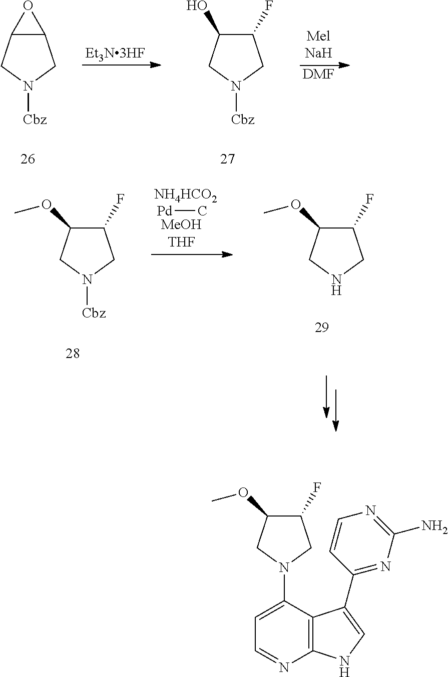

### trans-Benzyl 3-fluoro-4-hydroxypyrrolidine-1-carboxylate (27)

A mixture of 26 (900 mg, 4.11 mmol) and Et₃N.3HF (2.5 mL) was stirred at 110° C. for 20 hours. The reaction mixture was then cooled to room temperature and poured onto water. The product was extracted three times with ethyl acetate. The combined extracts were washed with sat. NaHCO₃ and then brine, dried (Na₂SO₄), filtered and concentrated to a brown paste. The crude product was purified by flash chromatography to give the title compound as a colorless paste (980 mg, quant.). ¹H-NMR (400 MHz, CDCl₃): δ7.29-7.39 (m, 5H), 5.14 (s, 2H), 4.37-4.49 (m, 1H), 3.51-3.84 (m, 4H), 2.29 (m, 1H).

### trans-Benzyl 3-fluoro-4-methoxypyrrolidine-1-carboxylate (28)

To a solution of 27 (0.50 g, 2.09 mmol) in DMF was added NaH (60% dispersion, 92 mg, 2.3 mmol). After 5 minutes of stirring at room temperature, methyl iodide (0.15 mL) was added. The reaction mixture was then stirred at room temperature for 3 hours prior to the addition of sat. NH₄Cl. The product was extracted with ethyl acetate, washed with brine, dried (Na2SO4), filtered and concentrated. The crude product was purified by flash chromatrography to give the title compound as a clear paste (359 mg, 68%). ¹H-NMR (400 MHz, CDCl₃): δ7.2-7.42 (m, 5H), 5.14 (s, 2H), 4.92-5.51 (m, 1H), 3.90-3.98 (m, 1H), 3.53-3.81 (m, 4H), 3.38 (s, 3H).

### trans-Benzyl 3-fluoro-4-methoxypyrrolidine (29)

A mixture of 28 (0.36 g, 1.42 mmol), ammonium formate (0.5 g) and palladium on carbon (10 wt %, 100 mg) in methanol (15 mL) and THF (15 mL) was stirred at room temperature for 2.5 hours. The reaction mixture was the filtered through celite and concentrated to give the title compound as a clear colorless paste. Due to its volatility, the product was not isolated before proceeding to the following step.

### 4-{4-[(3R,4R)-3-Fluoro-4-(methyloxy)pyrrolidin-1-yl]-1H-pyrrolo[2,3-b]pyridin-3-yl}pyrimidin-2-amine

¹H-NMR (400 MHz, MeOH-d₄): δ 8.17 (d, 1H), 8.01 (d, 1H), 7.68 (s, 1H), 6.94 (d, 1H), 6.61 (d, 1H), 3.92-3.99 (m, 1H), 3.56-3.62 (m, 2H), 3.47-3.53 (m, 1H), 3.41 (t, 1H), 3.34 (s, 3H), 3.17-3.22 (m, 1H). MS (ES) for C₁₆H₁₈FN₆O, found 329.1 (MH⁺).

### {(2S)-1-[3-(2-Aminopyrimidin-4-yl)-5-methyl-1H-pyrrolo[2,3-b]pyrid-4-yl]pyrrolidin-2-yl}methanol

¹H-NMR (400 MHz, MeOH-d₄): δ 8.21 (d, 1H), 7.98 (s, 1H), 7.67 (s, 1H), 6.94 (d, 1H), 3.97-4.05 (m, 1H), 3.33-3.41 (m, 3H), 3.12-3.20 (m, 1H), 2.36 (s, 3H), 1.75-1.92 (m, 4H) MS (ES) for C₁₇H₂₁N₆O, found 325.1 (MH⁺).

### Example 52

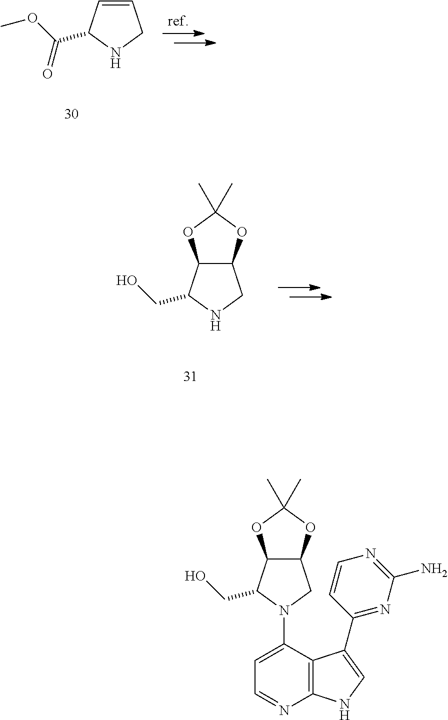

### ((3aR,4R,6aS)-2,2-dimethyltetrahydro-3aH-[1,3]dioxolo[4,5-c]pyrrol-4-yl)methanol (31)

The title compound was prepared according to procedures described in Aswan Science & Technology Bulletin 1991, 2, 39.

### {(3aR,4R,6aS)-5-[3-(2-Aminopyrimidin-4-yl)-1H-pyrrolo[2,3-b]pyridin-4-yl]-2,2-dimethyltetrahydro-3aH-[1,3]-dioxolo[4,5-c]pyrrol-4-yl}methanol

¹H-NMR (400 MHz, MeOH-d₄): δ 8.17 (d, 1H), 7.99 (d, 1H), 7.69 (s, 1H), 6.98 (d, 1H), 6.81 (d, 1H), 4.67-4.71 (m, 2H), 4.29 (t, 1H), 3.41-3.50 (m, 5H), 1.38 (s, 3H), 1.26 (s, 3H). MS (ES) for C₁₉H₂₂N₆O₃, found 383.2 (MH⁺).

### Example 53

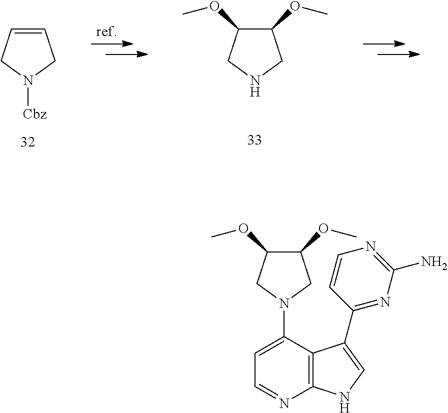

### cis-3,4-Dimethoxypyrrolidine (33)

The title compound was prepared according procedure described in PCT Int. Appl., 2005021554.

### 4-{-4-[(3R,4S)-3,4-Bis(methyloxy)pyrrolidin-1-yl]-1H-pyrrolo[2,3-b]pyridin-3-yl}pyrimidin-2-amine

¹H-NMR (400 MHz, MeOH-d₄): δ 8.17 (d, 1H), 7.93 (d, 1H), 7.55 (s, 1H), 6.86 (d, 1H), 6.50 (d, 1H), 3.89-3.94 (m, 2H), 3.42-3.48 (m, 2H), 3.32-3.36 (m, 2H), 3.34 (s, 6H). MS (ES) for C₁₇H₂₀N₆O₂, found 341.2 (MH⁺).

### Example 54

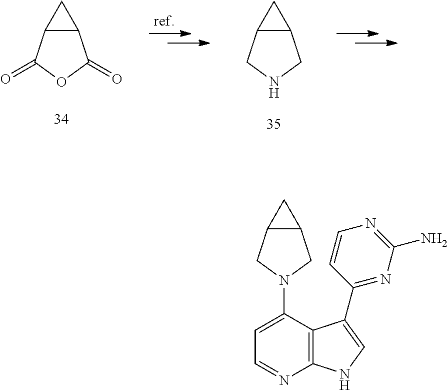

### 3-Azabicyclo[3.1.0]hexane (35).

The title compound was prepared according to procedures described in Bio. Med. Chem. Lett. 2005, 8, 2093.

Using analogous synthetic techniques as in Example 54, and substituting with the appropriate alternative starting materials, the following compounds were prepared. Alternative starting materials were obtained commercially unless otherwise indicated.

### 4-[4-(3-Azabicyclo[3.1.0]hex-3-yl)-1H-pyrrolo[2,3-b]pyridin-3-yl]pyrimidin-2-amine

¹H-NMR (400 MHz, MeOH-d₄): δ 8.19 (d, 1H), 7.96 (d, 1H), 7.63 (s, 1H), 6.81 (d, 1H), 6.62 (d, 1H), 3.54 (d, 2H), 3.12 (d, 2H), 1.46-1.51 (m, 2H), 0.64-0.69 (m, 1H), 0.49-0.56 (m, 1H). MS (ES) for C₁₆H₁₆N₆, found 293.2 (MH⁺).

### 1-[3-(2-Aminopyrimidin-4-yl)-1H-pyrrolo[2,3-b]pyridin-4-yl]imidazolidin-2-one

¹H-NMR (400 MHz, MeOH-d₄): δ 8.25 (d, 1H), 8.15 (d, 1H), 7.85 (s, 1H), 7.09 (d, 1H), 6.90 (d, 1H), 4.08 (t, 2H), 3.61 (t, 2H). MS (ES) for C₁₄H₁₃N₇O, found 296.1 (MH⁺).

### Example 55

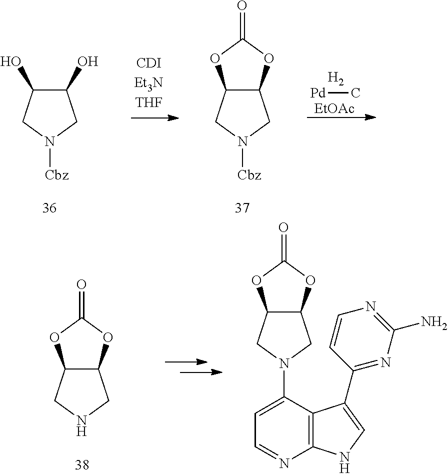

### cis-Benzyl 2-oxodihydro-3aH-[1,3]dioxolo[4,5-c]pyrrole-5(4H)-carboxylate (37)

To a solution of 36 (685 mg, 2.89 mmol) in THF (10 mL) was added carbonyldiimidazole (700 mg, 4.3 mmol) and triethylamine (0.4 mL). The resulting mixture was stirred at room temperature for 24 hours. Water was then added to the reaction and the product was extracted with ethyl acetate. The combined extracts were washed with brine, dried (Na₂SO₄), filtered and concentrated. The crude product was purified by flash chromatography to give the title compound as a colorless paste (863 mg, quant.). ¹H-NMR (400 MHz, CDCl₃): δ 7.31-7.41 (m, 5H), 5.16-5.19 (m, 2H), 4.05-4.18 (m, 2H), 3.43-3.49 (m, 4H).

### cis-Tetrahydro-3aH-[1,3]dioxolo[4,5-c]pyrrol-2-one (38)

A solution of 37 in ethyl acetate was stirred under a hydrogen atmosphere for 72 hours at room temperature. The resulting mixture was filtered through celite and concentrated to give the title compound. MS (ES) for C₅H₇NO₃, found 130 (MH⁺).

Using analogous synthetic techniques as in Example 54, and substituting with the appropriate alternative starting materials, the following compounds were prepared. Alternative starting materials were obtained commercially unless otherwise indicated.

### (3aR,6aS)-5-[3-(2-Aminopyrimidin-4-yl)-1H-pyrrolo[2,3-b]pyridin-4-yl]tetrahydro-3aH-[1,3]dioxolo[4,5-c]pyrrol-2-one

¹H-NMR (400 MHz, DMSO-d₆): δ 12.2 (s, 1H), 8.12 (d, 1H), 8.11 (d, 1H), 7.78 (s, 1H), 6.73 (d, 1H), 6.67 (d, 1H), 6.44 (s, 2H), 5.29 (d, 2H), 3.69 (d, 2H), 2.90 (d, 2H). MS (ES) for C₁₆H₁₄N₆O₃, found 339.1 (MH⁺).

### 4-[4-(2-Phenyl pyrrolidin-1-yl)-1H-pyrrolo[2,3-b]pyridin-3-yl]pyrimidin-2-amine

¹H-NMR (400 MHz, MeOH-d₄): δ 8.28 (d, 1H), 7.78 (d, 1H), 7.60 (s, 1H), 7.45-7.51 (m, 2H), 7.30-7.35 (m, 2H), 7.18-7.24 (m, 1H), 7.05 (d, 1H), 6.40 (d, 1H), 4.71 (t, 1H), 3.46-3.56 (m, 1H), 2.41-2.49 (m, 1H), 1.69-1.90 (m, 3H). MS (ES) for C₂₁H₂₀N₆, found 357.2 (MH⁺).

### Example 56

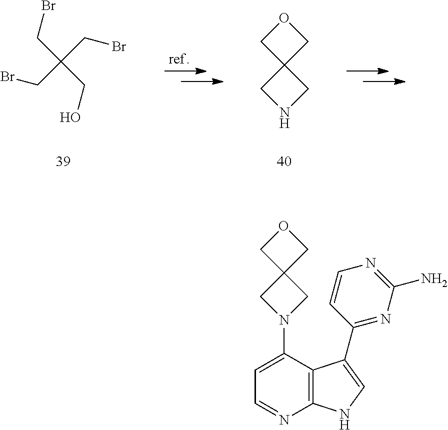

### 2-Oxa-6-azaspiro[3.3]heptane (40)

The title compound was prepared according to procedures described in Org. Lett. 2008, 16, 3525.

### 4-[4-(2-Oxa-6-azaspiro[3.3]hept-6-yl)-1H-pyrrolo[2,3-b]pyridin-3-yl]pyrimidin-2-amine

¹H-NMR (400 MHz, MeOH-d₄): δ 8.23 (d, 1H), 7.99 (d, 1H), 7.60 (s, 1H), 6.93 (d, 1H), 6.34 (d, 1H), 4.71 (s, 4H), 3.96 (s, 4H). MS (ES) for C₁₆H₁₆N₆O, found 309.0 (MH⁺).

### Example 57

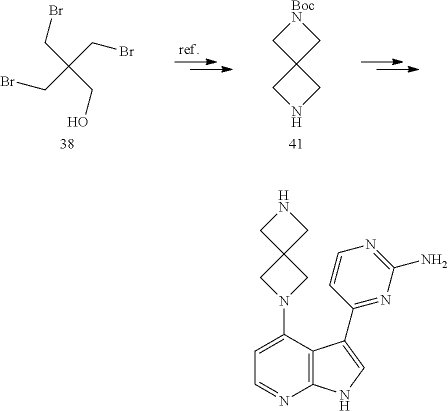

### tert-Butyl 2,6-diazaspiro[3.3]heptane-2-carboxylate (41)

The title compound was prepared according to procedures described in Org. Lett. 2008, 16, 3525.

Using analogous synthetic techniques as in Example 54, and substituting with the appropriate alternative starting materials, the following compounds were prepared. Alternative starting materials were obtained commercially unless otherwise indicated.

### 4-[4-(2,6-Diazaspiro[3.3]hept-2-yl)-1H-pyrrolo[2,3-b]pyridin-3-yl]pyrimidin-2-amine

¹H-NMR (400 MHz, MeOH-d₄): δ 8.22 (d, 1H), 8.00 (d, 1H), 7.63 (s, 1H), 6.95 (d, 1H), 6.34 (d, 1H), 4.15 (s, 4H), 3.98 (s, 4H). MS (ES) for C₁₆H₁₇N₇, found 308.1 (MH+).

### Example 58

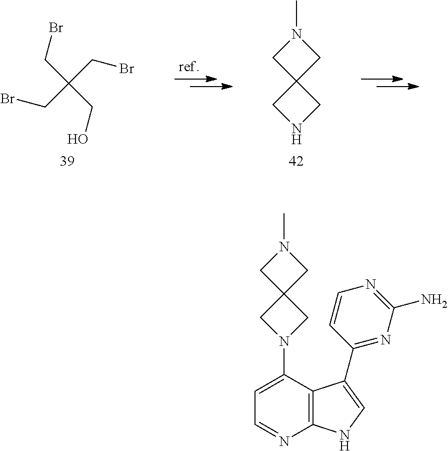

### 2-methyl-2,6-diazaspiro[3.3]heptane (42)

The title compound was prepared according to procedures described in Org. Lett. 2008, 16, 3525.

Using analogous synthetic techniques as in Example 54, and substituting with the appropriate alternative starting materials, the following compounds were prepared. Alternative starting materials were obtained commercially unless otherwise indicated.

### 4-[4-(6-Methyl-2,6-diazaspiro[3.3]hept-2-yl)-1H-pyrrolo[2,3-b]pyridin-3-yl]pyrimidin-2-amine

¹H-NMR (400 MHz, MeOH-d₄): δ 8.21 (d, 1H), 8.00 (d, 1H), 7.61 (s, 1H), 6.93 (d, 1H), 6.33 (d, 1H), 3.95 (s, 4H), 3.93 (s, 4H), 2.63 (s, 3H). MS (ES) for C₁₇H₁₉N₇, found 322.1 (MH⁺).

### 4-{4-[2-(Trifluoromethyl)phenyl]-1H-pyrrolo[2,3-b]pyridin-3-yl}pyrimidin-2-amine

¹H-NMR (400 MHz, MeOH-d₄): δ 8.32 (d, 1H), 7.93 (s, 1H), 7.74-7.79 (m, 1H), 7.71 (d, 1H), 7.50-7.59 (m, 2H), 7.26-7.32 (m, 1H), 7.09 (d, 1H), 6.14 (d, 1H). MS (ES) for C₁₈H₁₂F₃N₅, found 356.1 (MH⁺).

### 2-[3-(2-Aminopyrimidin-4-yl)-1H-pyrrolo[2,3-b]pyridin-4-yl]benzonitrile

¹H-NMR (400 MHz, MeOH-d₄): δ 8.42 (d, 1H), 7.99 (s, 1H), 7.89 (d, 1H), 7.64-7.73 (m, 2H), 7.49-7.56 (m, 2H), 7.22 (d, 1H), 6.44 (d, 1H). MS (ES) for C₁₈H₁₂N₆, found 313.0 (MH⁺).

### {4-[3-(2-aminopyrimidin-4-yl)-1H-pyrrolo[2,3-b]pyridin-4-yl]-1-methylpiperazin-2-yl}methanol

To benzyl 3-(hydroxymethyl)piperazine-1-carboxylate (2.00 g, 8.00 mmol) in DCM (20 mL) and AcOH (0.500 mL, 0.794 mmol) was added paraformaldehyde (0.500 g, 1.66 mmol) followed by sodium borohydride (0.500 g, 13.2 mmol). The mixture was stirred at ambient temperature for 4 h then diluted with aq. NaOH (1N), and EtOAc. The organic layer was washed with brine, dried with sodium sulfate, filtered, and concentrated. To the crude benzyl 3-(hydroxymethyl)-4-methylpiperazine-1-carboxylate (2.03 g) dissolved in EtOAc (20 mL) and methanol (20 mL) was added Pd/C (10%, 100 mg). The reaction mixture was stirred under a hydrogen atmosphere (balloon) for 48 h then exposed to air, filtered through celite, and concentrated. The crude (1-methylpiperazin-2-yl)methanol (1.04 g) was carried forward without further purification.

¹H NMR (400 MHz, DMSO-d₆) δ 8.21 (d, 1H), 8.10 (d, 1H), 7.74 (d, 1H), 6.99 (d, 1H), 6.68 (d, 1H), 6.40 (s, 2H), 4.47 (s, 1H), 3.50-3.39 (m, 4H), 3.19-3.13 (m, 2H), 2.67-2.50 (m, 3H), 2.20 (s, 3H). MS (ES) for C₁₇H₂₁N₇O, found 340.2 (MH+).

### 4-{-4-[(2S)-2-(fluoromethyl)pyrrolidin-1-yl]-1H-pyrrolo[2,3-b]pyridin-3-yl}pyrimidin-2-amine

To (S)-(1-(3-(2-aminopyrimidin-4-yl)-1-(phenylsulfonyl)-1H-pyrrolo[2,3-b]pyridin-4-yl)pyrrolidin-2-yl)methanol (200 mg, 0.471 mmol) in THF (5.0 mL) was added Bis(2-methoxyethyl)aminosulfur trifluoride (0.200 mL, 1.09 mmol). The reaction mixture was stirred for 30 min at ambient temperature before it was quenched with sodium bicarbonate (sat, aq.), extracted with EtOAc, washed with brine, dried with sodium sulfate, filtered, and concentrated. The crude (S)-(1-(3-(2-aminopyrimidin-4-yl)-1H-pyrrolo[2,3-b]pyridin-4-Apyrrolidin-2-yOmethanol was dissolved in MeOH (10 mL) and NaOH (4N, 40 mmol, 10 mL) then stirred for 30 min at ambient temperature before it was diluted with EtOAc, washed with brine, dried with sodium sulfate, filtered, and concentrated. The product was purified by reverse phase HPLC to provide the product (32 mg, 21%) as an off-white solid after lyphollization.

¹H NMR (400 MHz, DMSO-d₆) δ 8.20 (d, 1H), 8.10 (d, 1H), 7.75 (d, 1H), 6.96 (d, 1H), 6.72 (d, 1H), 6.43 (s, 2H), 4.86-4.79 (m, 1H), 4.73-4.68 (m, 1H), 3.01-2.91 (m 2H), 2.86-2.78 (m, 1H), 1.95-1.86 (m, 1H), 1.74-1.57 (m, 2H), 1.46-1.36 (m, 1H). MS (ES) for C₁₆H₁₇FN₆, found 313.2 (MH+).

### 4-[4-(5-methylhexahydropyrrolo[3,4-b]pyrrol-1(2H)-yl)-1H-pyrrolo[2,3-b]pyridin-3-yl]pyrimidin-2-amine

¹H-NMR (400 MHz, DMSO): δ 8.14 (d, 1H), 8.0 (d, 1H), 7.68 (s, 1H), 6.84 9d, 1H), 6.55 (d, 1H), 6.44 9s, 2H), 4.1 (m, 1H), 3.3 (m, 1H), 3.0 (m, 1H), 2.68 (m, 1H), 2.48 (d, 2H), 2.2-2.1 (m, 2H), 2.1 (s, 3H), 1.92 (m, 1H), 1.6 (m, 1H). MS (EI) for C₁₈H₂₁N₇, found 336.2 (MH⁺).

### (3R)-1-[3-(2-aminopyrimidin-4-yl)-1H-pyrrolo[2,3-b]pyridin-4-yl]pyrrolidine-3-carboxylic acid

¹H-NMR (400 MHz, DMSO): δ 8.13 (d, 1H), 7.94 (d, 1H), 7.58 (s, 1H), 6.78 (d, 1H), 6.44 (d, 1H), 6.42 (s, 2H), 3.32 (t, 1H), 3.25 (t, 1H), 3.06 (t, 2H), 2.76 (q, 1H), 2.0-1.84 (m, 2H). MS (EI) for C₁₆H₁₆N₆O₂, found 325.1 (MH⁺).

### (3R)-1-[3-(2-aminopyrimidin-4-yl)-1H-pyrrolo[2,3-b]pyridin-4-yl]pyrrolidine-3-carboxamide

¹H-NMR (400 MHz, DMSO): δ 8.14 (d, 1H), 7.97 (d, 1H), 7.62 (s, 1H), 7.4 (s, 1H), 6.9 (s, 1H), 6.8 (d, 1H), 6.47 (d, 1H), 6.44 (s, 2H), 3.38 (t, 1H), 3.2 (t, 1H), 3.1 (t, 2H), 2.9 (t, 1H), 2.0-1.85 (m, 2H). MS (EI) for C₁₆H₁₇N₇O, found 324.1 (MH⁺).

### Ethyl (3S)-1-[3-(2-aminopyrimidin-4-yl)-1H-pyrrolo[2,3-b]pyridin-4-yl]pyrrolidine-3-carboxylate

¹H-NMR (400 MHz, DMSO): δ 8.12 (d, 1H), 7.99 (d, 1H), 7.64 (s, 1H), 6.78 (d, 1H), 6.52 (d, 1H), 6.44 (s, 2H), 4.05 (q, 2H), 3.32 (m, 2H), 3.2-3.0 (m, 3H), 2.04 (m, 2H), 1.16 (t, 3H). MS (EI) for C₁₈H₂₀N₆O₂, found 353.2 (MH⁺).

### (3S)-1-[3-(2-aminopyrimidin-4-yl)-1H-pyrrolo[2,3-b]pyridin-4-yl]-N-methylpyrrolidine-3-carboxamide

¹H-NMR (400 MHz, CD₃OD): δ 8.18 (d, 1H), 7.98 (d, 1H), 7.66 (s, 1H), 6.95 (d, 1H), 6.6 (d, 1H), 3.47 (t, 1H), 3.38-3.2 (m, 3H), 2.98 (t, 1H), 2.7 (s, 3H), 2.1-2.0 (m, 2H). MS (EI) for C₁₇H₁₉N₇O, found 338.2 (MH⁺).

### 4-[4-(1,3-dihydro-2H-isoindol-2-yl)-1H-pyrrolo[2,3-b]pyridin-3-yl]pyrimidin-2-amine

¹H-NMR (400 MHz, DMSO): δ 12.0 (s, 1H), 8.04 (d, 1H), 8.0 (d, 1H), 7.67 (s, 1H), 7.22 (s, 4H), 6.75 (d, 1H), 6.68 (d, 1H), 6.45 (s, 2H), 4.56 (s, 4H). MS (EI) for C₁₉H₁₆N₆, found 329.1 (MH⁺).

### (3S)-1-[3-(2-aminopyrimidin-4-yl)-1H-pyrrolo[2,3-b]pyridin-4-yl]pyrrolidine-3-carboxamide

¹H-NMR (400 MHz, DMSO): δ 11.9 (s, 1H), 8.15 (d, 1H), 7.98 (d, 1H), 7.6 (s, 1H), 7.4 9s, 1H), 6.9 9s, 1H), 6.8 (d, 1H), 6.48 (d, 1H), 6.43 9s, 2H), 3.38 (t, 1H), 3.2 9t, 1H), 3.12 (t, 2H), 2.9 (m, 1H), 2.0-1.84 (m, 2H). MS (EI) for C₁₆H₁₇N₇O, found 324.1 (MH⁺).

### 4-{4-[(3R)-3-phenylpyrrolidin-1-yl]-1H-pyrrolo[2,3-b]pyridin-3-yl}pyrimidin-2-amine

¹H-NMR (400 MHz, DMSO): δ 11.8 (s, 1H), 8.12 (d, 1H), 7.94 (d, 1H), 7.52 (s, 1H), 7.3-7.15 (m, 5H), 6.75 (d, 1H), 6.48 (s, 2H), 6.46 (d, 1H), 3.57 (t, 1H), 3.4-3.26 (m, 3H), 3.17 (t, 1H), 2.24-2.16 (m, 1H), 1.9-1.8 (m, 1H). MS (EI) for C₂₁H₂₀N₆, found 357.2 (MH⁺).

### 4-[4-(3,4-dihydroisoquinolin-2(1H)-yl)-1H-pyrrolo[2,3-b]pyridin-3-yl]pyrimidin-2-amine

¹H-NMR (400 MHz, DMSO): δ 12.08 (s, 1H), 8.1 (d, 1H), 7.82 (d, 1H), 7.78 (s, 1H), 7.18-7.08 (m, 3H), 6.96 (d, 2H), 6.92 (d, 1H), 6.76 (d, 1H), 6.48 (s, 2H), 4.58 (s, 2H), 3.83 (t, 2H), 2.96 (t, 2H). MS (EI) for C₂₀H₁₈N₆, found 343.2 (MH⁺).

### 4-[4-(2,3-dihydro-1H-indol-1-yl)-1H-pyrrolo[2,3-b]pyridin-3-yl]pyrimidin-2-amine

¹H-NMR (400 MHz, DMSO): δ 12.18 (s, 1H), 8.19 (d, 1H), 7.84 (s, 1H), 7.83 (d, 1H), 7.08 (d, 1H), 7.03 (d, 1H), 6.74 (d, 1H), 6.72 (t, 1H), 6.58 (t, 1H), 6.39 (d, 1H), 6.2 (s, 2H), 3.9 (t, 2H), 3.06 (t, 2H). MS (EI) for C₁₉H₁₆N₆, found 329.1 (MH⁺).

### [(2R,3S)-1-[3-(2-aminopyrimidin-4-O-1H-pyrrolo[2,3-b]pyridin-4-yl]-3-(methyloxy)pyrrolidin-2-yl]methanol

¹H-NMR (400 MHz, DMSO): δ 8.13 (d, 1H), 8.02 (d, 1H), 7.7 (s, 1H), 6.86 9d, 1H), 6.72 (d, 1H), 6.36 (s, 2H), 4.06 (m, 1H), 3.98 (m, 1H), 3.6 (m, 2H), 3.54-3.38 (m, 2H), 3.24 9s, 3H), 2.16-2.04 (m, 1H), 1.76-1.66 (m, 1H). MS (EI) for C₁₇H₂₀N₆O₂, found 341.2 (MH⁺).

### {(2R,3S)-1-[3-(2-aminopyrimidin-4-yl)-1H-pyrrolo[2,3-b]pyridin-4-yl]-3-fluoropyrrolidin-2-yl}methanol

¹H-NMR (400 MHz, DMSO): δ 8.09 (d, 1H), 8.01 (d, 1H), 7.63 (s, 1H), 6.78 9d, 1H), 6.68 9d, 1H), 6.4 (s, 2H), 5.45-5.3 (m, 1H), 5.0 (b, 1H), 3.95-3.8 (m, 1H), 3.75-3.6 (m, 2H), 3.4-3.2 (m, 2H), 2.96 (m, 1H), 2.0-1.8 (m, 2H). MS (EI) for C₁₆H₁₇FN₆O, found 329.1 (MH⁺).

### {3-[3-(2-aminopyrimidin-4-yl)-1H-pyrrolo[2,3-b]pyridin-4-yl]phenyl}methanol

¹H-NMR (400 MHz, DMSO): δ 8.34 (d, 1H), 7.93 (s, 1H), 7.67 (d, 1H), 7.3-7.2 (m, 3H), 7.15-7.09 (m, 2H), 6.06 (s, 2H), 5.78 (d, 1H), 4.4 (s, 2H). MS (EI) for C₁₈H₁₅N₅O, found 318.1 (MH⁺).

### 4-(4-phenyl-1H-pyrrolo[2,3-b]pyridin-3-yl)pyrimidin-2-amine

¹H-NMR (400 MHz, DMSO): δ 12.82 (s, 1H), 8.42 (d, 1H), 8.3 9s, 1H), 7.9 (d, 1H), 7.52 (s, 2H), 7.4-7.25 (m, 5H), 7.22 (d, 1H), 6.22 (d, 1H). MS (EI) for C₁₇H₁₃N₅, found 288.1 (MH⁺).

### 4-{4-[(3R)-3-(ethyloxy)pyrrolidin-1-yl]-1H-pyrrolo[2,3-b]pyridin-3-yl}pyrimidin-2-amine

¹H-NMR (400 MHz, DMSO): δ 8.16 (d, 1H), 7.96 (d, 1H), 7.58 (s, 1H), 6.76 (d, 1H), 6.48 (s, 2H), 6.44 9d, 1H), 4.02 (m, 1H), 3.45-3.25 (m, 3H), 3.22-3.05 (m, 3H), 2.0-1.9 (m, 1H), 1.85-1.75 (m, 1H), 1.04 (t, 3H). MS (EI) for C₁₇H₂₀N₆O, found 325.1 (MH⁺).

### 4-[4-{(3R)-3-[(2,2-difluoroethyl)oxy]pyrrolidin-1-yl}-1H-pyrrolo[2,3-b]pyridin-3-yl)pyrimidin-2-amine

¹H-NMR (400 MHz, DMSO): δ 8.16 (d, 1H), 7.98 (d, 1H), 7.64 (s, 1H), 6.7 (d, 1H), 6.55 (s, 2H), 6.48 (d, 1H), 4.9 (m, 1H), 4.8-4.6 (m, 2H), 4.2 (m, 1H), 3.4-3.25 (m, 2H), 3.25-3.15 (m, 1H), 3.0 (d, 1H), 2.0-1.85 (m, 1H), 1.75-1.65 (m, 1H). MS (EI) for C₁₇H₁₈F₂N₆O, 361.1 (MH⁺).

### 4-(5-fluoro-1H-pyrrolo[2,3-b]pyridin-3-yl)pyrimidin-2-amine

¹H-NMR (400 MHz, DMSO): δ 12.38 (s, 1H), 8.8 (d, 1H), 8.48 (d, 1H), 8.28 (s, 1H), 8.15 (d, 1H), 7.05 (d, 1H), 6.6 (s, 2H). MS (EI) for C₁₁H₈FN₅, found 230.0 (MH+).

### 4-[4-(1H-pyrazol-4-yl)-1H-pyrrolo[2,3-b]pyridin-3-yl]pyrimidin-2-amine

¹H-NMR (400 MHZ, DMSO): δ 8.25 (d, 1H), 7.88 (d, 1H), 7.83 (s, 1H), 7.52 (s, 2H), 7.17 (d, 1H), 6.32 9s, 2H), 6.0 (d, 1H). MS (EI) for C₁₄H₁N₇, found 278.1 (MH+).

### 4-{4-[6-(methyloxy)pyridin-3-yl]-1H-pyrrolo[2,3-b]pyridin-3-yl}pyrimidin-2-amine

¹H-NMR (400 MHz, DMSO): δ 8.35 (d, 1H), 8.04 (d, 1H), 7.98 (s, 1H), 7.84 (d, 1H), 7.56 (dd, 1H), 7.15 (d, 1H), 6.74 (d, 1H), 6.1 (d, 1H), 6.02 (s, 2H), 3.82 (s, 3H). MS (EI) for C₁₇H₁₄N₆O, found 319.1 (MH+).

### 4-{4-[2-(methyloxy)pyridin-4-yl]-1H-pyrrolo[2,3-b]pyridin-3-yl}pyrimidin-2-amine

¹H-NMR (400 MHz, DMSO): δ 8.38 (d, 1H), 8.07 (d, 1H), 8.02 (s, 1H), 7.83 9d, 1H), 7.16 (d, 1H), 6.86 (d, 1H), 6.62 (s, 1H), 6.1 (d, 1H), 5.96 (s, 2H), 3.8 (s, 3H). MS (EI) for C₁₇H₁₄N₆O, found 319.1 (MH₊).

4-[3-(2-aminopyrimidin-4-yl)-1H-pyrrolo[2,3-b]pyridin-4-yl]pyridin-2-ol

¹H-NMR (400 MHz, DMSO): δ 8.37 (d, 1H), 8.01 (s, 1H), 7.96 (d, 1H), 7.22 (d, 1H), 7.13 (d, 1H), 6.32 (d, 1H), 6.2 (s, 1H), 6.12 9s, 2H), 5.98 (d, 1H). MS (EI) for C₁₆H₁₂N₆O, found 305.1 (MH⁺).

4-(4-((3S,4R)-3-amino-4-phenylpyrrolidin-1-yl)-1H-pyrrolo[2,3-b]pyridin-3-yl)pyrimidin-2-amine ¹H-NMR (400 MHz, DMSO-d₆): δ 11.8 (s, 1H), 8.11 (d, 1H), 7.95 (d, 1H), 7.50 (s, 1H), 7.26 (m, 5H), 6.75 (d, 1H), 6.48 (s, 2H), 6.44 (d, 1H), 3.61 (dd, 1H), 3.47 (dd, 1H), 3.32 (m, 2H), 2.98 (m, 2H). MS (ES) for C₂₁H₂₁N₇, found 372.2 (MH+).

### (N-((1-(3-(2-aminopyrimidin-4-yl)-1H-pyrrolo[2,3-b]pyridin-4-yl)pyrrolidin-2-yl)methyl)-2-(dimethylamino)acetamide

¹H-NMR (400 MHz, DMSO-d₆): δ 11.8 (s, 1H), 8.14 (d, 1H), 7.98 (d, 1H), 7.85 (br s, 1H), 7.61 (s, 1H), 6.83 (d, 1H), 6.79 (d, 1H), 6.37 (s, 2H), 3.89 (m, 1H), 3.42 (m, 1H), 3.23 (m, 1H), 3.11 (m, 1H), 2.89 (m, 1H), 2.87 (s, 2H), 2.19 (s, 6H), 1.97 (m, 1H), 1.65 (m, 2H), 1.53 (m, 1H). MS (ES) for C₂₀H₂₆N₈O, found 395.2 (MH+).

### (R)-4-(4-(3-fluoropyrrolidin-1-yl)-1H-pyrrolo[2,3-b]pyridin-3-yl)pyrimidin-2-amine

¹H-NMR (400 MHz, DMSO-d₆): δ 11.9 (s, 1H), 8.13 (d, 1H), 7.98 (d, 1H), 7.59 (s, 1H), 6.77 (d, 1H), 6.49 (d, 1H), 6.46 (s, 2H), 5.29 (m, 1H), 3.43 (m, 1H), 3.30 (m, 2H), 3.13 (m, 1H), 2.05 (m, 2H). MS (ES) for C₁₅H₁₅FN₆, found 299.2 (MH+).

### (S)-1-(3-(2-aminopyrimidin-4-yl)-1H-pyrrolo[2,3-b]pyridin-4-yl)pyrrolidine-2-carboxamide

¹H-NMR (400 MHz, DMSO-d₆): δ 12.0 (s, 1H), 8.21 (d, 1H), 8.18 (br s, 1H), 7.98 (d, 1H), 7.68 (d, 1H), 7.01 (br s, 1H), 6.99 (d, 1H), 6.60 (d, 1H), 6.20 (s, 2H), 4.26 (dd, 1H), 3.12 (1H, dt), 2.83 (dt, 1H), 2.27 (m, 1H), 1.82 (m, 1H), 1.62 (m, 2H). MS (ES) for C₁₆H₁₇N₇O, found 324.2 (MH+).

### (3R,5S)-1-(3-(2-aminopyrimidin-4-yl)-1H-pyrrolo[2,3-b]pyridin-4-yl)-5-(hydroxymethyl)pyrrolidin-3-ol

¹H-NMR (400 MHz, DMSO-d₆): δ 11.8 (s, 1H), 8.09 (d, 1H), 7.94 (d, 1H), 7.57 (d, 1H), 6.84 (d, 1H), 6.62 (d, 1H), 6.36 (s, 2H), 4.75 (t, 1H), 4.63 (d, 1H), 4.06 (m, 2H), 3.55 (m, 1H), 3.49 (m, 1H), 3.22 (dd, 1H), 2.88 (d, 1H), 1.89 (m, 2H). MS (ES) for C₁₆H₁₈N₆O₂, found 327.2 (MH+).

### (2S,4S)-1-(3-(2-aminopyrimidin-4-yl)-1H-pyrrolo[2,3-b]pyridin-4-yl)-4-hydroxypyrrolidine-2-carboxamide

¹H-NMR (400 MHz, DMSO-d₆): δ 11.9 (s, 1H), 8.20 (d, 1H), 8.06 (br s, 1H), 7.94 (d, 1H), 7.63 (s, 1H), 7.03 (bs, 1H), 6.96 (d, 1H), 6.51 (d, 1H), 6.22 (s, 2H), 5.05 (br s, 1H), 4.31 (t, 1H), 4.01 (m, 1H), 3.11 (dd, 1H), 2.85 (dd, 1H), 2.43 (dt, 1H), 1.67 (dt, 1H). MS (ES) for C₁₆H₁₇N₇O₂, found 340.2 (MH+).

### (S)-2-(1-(3-(2-aminopyrimidin-4-yl)-1H-pyrrolo[2,3-b]pyridin-4-yl)pyrrolidin-2-yl)acetamide

¹H-NMR (400 MHz, DMSO-d₆): δ 11.9 (s, 1H), 8.12 (d, 1H), 7.98 (d, 1H), 7.61 (d, 1H), 7.35 (br s, 1H), 6.84 (br s, 1H), 6.79 (d, 1H), 6.60 (d, 1H), 6.37 (s, 2H), 3.98 (m, 1H), 3.17 (m, 1H), 2.86 (m, 1H), 2.37 (d, 1H), 2.15 (d, 1H), 2.04 (m, 1H), 1.65 (m, 2H), 1.56 (m, 1H). MS (ES) for C₁₇H₁₉N₇O, found 338.2 (MH+).

### (3S,5S)-1-(3-(2-aminopyrimidin-4-yl)-1H-pyrrolo[2,3-b]pyridin-4-yl)-5-(hydroxymethyl)pyrrolidin-3-ol

¹H-NMR (400 MHz, DMSO-d₆): δ 11.8 (s, 1H), 8.09 (d, 1H), 7.91 (d, 1H), 7.56 (s, 1H), 6.80 (d, 1H), 6.55 (d, 1H), 6.34 (s, 2H), 4.95 (d, 1H), 4.76 (t, 1H), 4.03 (m, 1H), 3.85 (m, 1H), 3.41 (m, 2H), 3.18 (dd, 1H), 2.97 (dd, 1H), 2.13 (dt, 1H), 1.64 (dt, 1H). MS (ES) for C₁₆H₁₈N₆O₂, found 327.2 (MH+).

### (S)-1-(3(2-aminopyrimidin-4-yl)-1H-pyrrolo[2,3-b]pyridin-4-yl)-N-methylpyrrolidine-2-carboxamide

¹H-NMR (400 MHz, DMSO-d₆): δ 12.0 (s, 1H), 8.23 (q, 1H), 8.19 (d, 1H), 7.97 (d, 1H), 7.71 (s, 1H), 7.09 (d, 1H), 6.54 (d, 1H), 6.22 (s, 2H), 4.25 (t, 1H), 3.23 (dt, 1H), 2.87 (dt, 1H), 2.60 (d, 3H), 2.24 (m, 1H), 1.84 (m, 1H), 1.69 (m, 1H), 1.62 (m, 1H). MS (ES) for C₁₇H₁₉N₇O, found 338.2 (MH+).

### (2S,4S)-1-(3-(2-aminopyrimidin-4-yl)-1H-pyrrolo[2,3-b]pyridin-4-yl)-4-fluoropyrrolidine-2-carboxamide

¹H-NMR (400 MHz, DMSO-d₆): δ 12.0 (s, 1H), 8.30 (br s, 1H), 8.20 (d, 1H), 8.03 (d, 1H), 7.72 (d, 1H), 7.12 (br s, 1H), 6.95 (d, 1H), 6.76 (d, 1H), 6.14 (s, 2H), 5.10 (m, 1H), 4.47 (dd, 1H), 3.41 (m, 1H), 3.04 (ddd, 1H), 2.58 (m, 1H), 2.15 (m, 1H). MS (ES) for C₁₆H₁₆FN₇O, found 342.2 (MH+).

### ((2S,4S)-1-(3-(2-aminopyrimidin-4-yl)-1H-pyrrolo[2,3-b]pyridin-4-yl)-4-fluoropyrrolidin-2-yl)methanol

¹H-NMR (400 MHz, DMSO-d₆): δ 12.0 (s, 1H), 8.10 (d, 1H), 8.02 (d, 1H), 7.69 (d, 1H), 6.85 (d, 1H), 6.72 (d, 1H), 6.37 (s, 2H), 5.23 (m, 1H), 4.79 (t, 1H), 3.89 (m, 1H), 3.51 (m, 1H), 3.37 (m, 2H), 3.24 (m, 1H), 2.27 (m, 1H), 2.08 (m, 1H). MS (ES) for C₁₆H₁₇FN₆O, found 329.2 (MH+).

### (R)-3-(3-(2-aminopyrimidin-4-yl)-1H-pyrrolo[2,3-b]pyridin-4-yl)thiazolidine-4-carboxamide

¹H-NMR (400 MHz, CDCl₃): δ 8.27 (d, 1H), 8.13 (d, 1H), 7.60 (s, 1H), 6.96 (d, 1H), 6.79 (d, 1H), 4.92 (m, 1H), 4.20 (d, 1H), 4.09 (d, 1H), 3.50 (m, 1H), 3.39 (m, 1H). MS (ES) for C₁₅H₁₅N₇OS, found 342.1 (M+H).

### ((2S,4R)-1-(3-(2-aminopyrimidin-4-yl)-1H-pyrrolo[2,3-b]pyridin-4-yl)-4-fluoropyrrolidin-2-yl)methanol

¹H-NMR (400 MHz, DMSO-d₆): δ 11.9 (s, 1H), 8.10 (d, 1H), 7.98 (d, 1H), 7.64 (s, 1H), 6.87 (d, 1H), 6.67 (d, 1H), 6.38 (s, 2H), 5.17 (m, 1H), 4.82 (t, 1H), 4.06 (m, 1H), 3.47 (m, 2H), 3.38 (m, 1H), 3.22 (m, 1H), 2.12 (m, 2H). MS (ES) for C₁₆H₁₇FN₆O, found 329.2 (MH+).

### ((2S,4S)-1-(3-(2-aminopyrimidin-4-yl)-1H-pyrrolo[2,3-b]pyridin-4-yl)-4-methoxypyrrolidin-2-yl)methanol

¹H-NMR (400 MHz, DMSO-d₆): δ 11.9 (s, 1H), 8.12 (d, 1H), 7.97 (d, 1H), 7.62 (s, 1H), 6.83 (d, 1H), 6.62 (d, 1H), 6.37 (s, 2H), 4.72 (t, 1H), 3.84 (m, 2H), 3.76 (m, 2H), 3.27 (dd, 1H), 3.11 (d, 1H), 3.10 (s, 3H), 2.21 (dt, 1H), 1.75 (dt, 1H). MS (ES) for C₁₇H₂₀N₆O₂, found 341.2 (MH+).

### (S)-(1-(3-(2-aminopyrimidin-4-yl)-1H-pyrrolo[2,3-b]pyridin-4-yl)-4,4-difluoropyrrolidin-2-yl)methanol

¹H-NMR (400 MHz, DMSO-d₆): δ 12.1 (s, 1H), 8.14 (d, 1H), 8.06 (d, 1H), 7.73 (s, 1H), 6.92 (d, 1H), 6.71 (d, 1H), 6.41 (s, 2H), 4.91 (m, 1H), 3.98 (m, 1H), 3.68 (m, 1H), 3.40 (m, 2H), 3.31 (m, 1H), 2.42 (m, 2H). MS (ES) for C₁₆H₁₆F₂N₆O, found 347.2 (MH+).

### (3S,5S)-1-(3-(2-aminopyrimidin-4-yl)-1H-pyrrolo[2,3-b]pyridin-4-yl)-5-(hydroxymethyl)-3-(trifluoromethyl)pyrrolidin-3-ol

¹H-NMR (400 MHz, DMSO-d₆): δ 12.0 (s, 1H), 8.10 (d, 1H), 8.00 (d, 1H), 7.67 (d, 1H), 6.84 (d, 1H), 6.61 (d, 1H), 6.54 (s, 1H), 6.40 (s, 2H), 4.84 (t, 1H), 3.91 (m, 1H), 3.43 (m, 3H), 3.30 (m, 1H), 2.29 (dd, 1H), 2.05 (dd, 1H). MS (ES) for C₁₇H₁₇F₃N₆O₂, found 395.1 (MH+).

### ((2S,4S)-1-(3-(2-aminopyrimidin-4-yl)-1H-pyrrolo[2,3-b]pyridin-4-yl)-4-methoxy-4-(trifluoromethyl)pyrrolidin-2-yl)methanol

¹H-NMR (400 MHz, DMSO-d₆): δ 12.0 (s, 1H), 8.13 (d, 1H), 8.03 (d, 1H), 7.68 (s, 1H), 6.87 (d, 1H), 6.69 (d, 1H), 6.43 (s, 2H), 4.90 (m, 1H), 3.94 (m, 1H), 3.45 (m, 4H), 3.26 (s, 3H), 2.29 (m, 2H). MS (ES) for C₁₈H₁₉F₃N₆O₂, found 409.2 (MH+).

### 3-(2-aminopyrimidin-4-yl)-5-methoxy-N,N-dimethyl-1H-pyrrolo[2,3-b]pyridin-4-amine

¹H-NMR (400 MHz, DMSO-d₆): δ 11.9 (s, 1H), 8.14 (d, 1H), 7.98 (s, 1H), 7.76 (s, 1H), 6.95 (d, 1H), 6.32 (s, 2H), 3.79 (s, 3H), 2.74 (s, 6H). MS (ES) for C₁₄H₁₆N₆O, found 285.2 (MH+).

### 4-(4-chloro-5-methoxy-1H-pyrrolo[2,3-b]pyridin-3-yl)pyrimidin-2-amine

¹H-NMR (400 MHz, DMSO-d₆): δ 12.3 (s, 1H), 8.25 (s, 1H), 8.19 (d, 1H), 7.90 (s, 1H), 6.82 (d, 1H), 6.47 (s, 2H), 3.96 (s, 3H). MS (ES) for C₁₂H₁₀ClN₅O, found 276.1 (MH+).

### 4-(5-methoxy-4-(pyrrolidin-1-yl)-1H-pyrrolo[2,3-b]pyridin-3-yl)pyrimidin-2-amine

¹H-NMR (400 MHz, DMSO-d₆): δ 11.9 (s, 1H), 8.14 (d, 1H), 8.00 (s, 1H), 7.72 (d, 1H), 6.86 (d, 1H), 6.37 (s, 2H), 3.79 (s, 3H), 3.26 (m, 4H), 1.75 (m, 4H). MS (ES) for C₁₆H₁₈N₆O, found 311.2 (MH+).

### 3-(2-aminopyrimidin-4-yl)-N-(2-(pyrrolidin-1-yl)ethyl)-1H-pyrrolo[2,3-b]pyridin-4-amine

¹H-NMR (400 MHz, DMSO-d₆): δ 11.9 (s, 1H), 10.3 (t, 1H), 8.11 (s, 1H), 8.07 (d, 1H), 7.83 (d, 1H), 7.15 (br s, 2H), 7.08 (d, 1H), 6.14 (d, 1H), 3.31 (m, 2H), 2.80 (m, 2H), 2.53 (m, 4H), 1.76 (m, 4H). MS (ES) for C₁₇H₂₁N₇, found 324.2 (MH+).

### N¹-(3-(2-aminopyrimidin-4-yl)-1H-pyrrolo[2,3-b]pyridin-4-yl)-N²,N²-diethylethane-1,2-diamine

¹H-NMR (400 MHz, DMSO-d₆): δ 11.9 (s, 1H), 10.1 (t, 1H), 8.09 (s, 1H), 8.07 (d, 1H), 7.83 (d, 1H), 7.08 (d, 1H), 7.00 (s, 2H), 6.17 (d, 1H), 3.33 (m, 2H), 2.74 (t, 2H), 2.57 (q, 4H), 0.98 (t, 6H). MS (ES) for C₁₇H₂₃N₇, found 326.2 (MH+).

### N¹-(3-(2-aminopyrimidin-4-yl)-1H-pyrrolo[2,3-b]pyridin-4-yl)-N³,N³-dimethylpropane-1,3-diamine

¹H-NMR (400 MHz, DMSO-d₆): δ 11.8 (s, 1H), 9.74 (t, 1H), 8.10 (d, 1H), 8.03 (s, 1H), 7.82 (d, 1H), 7.08 (d, 1H), 6.56 (s, 2H), 6.13 (d, 1H), 3.29 (m, 2H), 2.30 (t, 2H), 2.12 (s, 6H), 1.81 (quintet, 2H). MS (ES) for C₁₆H₂₁N₁₇, found 312.2 (MH+).

### 3-(2-aminopyrimidin-4-yl)-N-(2-(piperidin-1-yl)ethyl)-1H-pyrrolo[2,3-b]pyridin-4-amine

¹H-NMR (400 MHz, DMSO-d₆): δ 11.8 (s, 1H), 10.1 (s, 1H), 8.08 (m, 2H), 7.82 (s, 1H), 7.07 (d, 1H), 7.04 (s, 2H), 6.12 (d, 1H), 3.29 (m, 2H), 2.65 (m, 2H), 2.39 (m, 4H), 1.51 (m, 4H), 1.40 (m, 2H). MS (ES) for C₁₈H₂₃N₇, found 338.2 (MH+).

### N¹-(3-(2-aminopyrimidin-4-yl)-1H-pyrrolo[2,3-b]pyridin-4-yl)-N²-methylethane-1,2-diamine hydrochloride

¹H-NMR (400 MHz, DMSO-d₆): δ 13.3 (s, 1H), 11.8 (s, 1H), 9.10 (s, 2H), 8.49 (s, 1H), 8.29 (d, 1H), 8.12 (d, 1H), 7.68 (br s, 1H), 7.40 (d, 1H), 6.82 (d, 1H), 3.99 (q, 2H), 3.20 (m, 2H), 2.57 (t, 3H). MS (ES) for C₁₄H₁₇N₇, found 284.2 (MH+).

### N¹-(3-(2-aminopyrimidin-4-yl)-1H-pyrrolo[2,3-b]pyridin-4-yl)ethane-1,2-diamine hydrochloride

¹H-NMR (400 MHz, DMSO-d₆): δ 13.5 (br s, 1H), 10.7 (s, 1H), 8.55 (s, 1H), 8.31 (d, 1H), 8.26 (s, 3H), 8.10 (d, 1H), 8.06 (br s, 1H), 7.46 (d, 1H), 6.80 (d, 1H), 3.91 (m, 2H), 3.09 (m, 2H). MS (ES) for C₁₃H₁₅N₇, found 270.2 (MH+).

### N¹-(3-(2-aminopyrimidin-4-yl)-1H-pyrrolo[2,3-b]pyridin-4-yl)propane-1,3-diamine hydrochloride

¹H-NMR (400 MHz, DMSO-d₆): δ 13.4 (s, 1H), 10.9 (s, 1H), 8.51 (s, 1H), 8.29 (d, 1H), 8.06 (s, 3H), 8.05 (d, 1H), 7.84 (br s, 1H), 7.43 (d, 1H), 6.73 (d, 1H), 3.75 (q, 2H), 2.86 (m, 2H), 1.93 (quintet, 2H). MS (ES) for C₁₄H₁₇N₇, found 284.2 (MH+).

### 3-(2-aminopyrimidin-4-yl)-N-(2-fluoroethyl)-1H-pyrrolo[2,3-b]pyridin-4-amine

¹H-NMR (400 MHz, DMSO-d₆): δ 11.9 (s, 1H), 9.96 (t, 1H), 8.11 (d, 1H), 8.10 (s, 1H), 7.85 (d, 1H), 7.12 (d, 1H), 6.42 (s, 2H), 6.22 (d, 1H), 4.79 (t, 1H), 4.67 (t, 1H), 3.67 (q, 1H), 3.60 (q, 1H). MS (ES) for C₁₃H₁₃FN₆, found 273.1 (MH+).

### (R)-2-(3-(2-aminopyrimidin-4-yl)-1H-pyrrolo[2,3-b]pyridin-4-ylamino)butan-1-ol

¹H-NMR (400 MHz, DMSO-d₆): δ 11.8 (s, 1H), 10.2 (d, 1H), 8.08 (s, 1H), 8.07 (d, 1H), 7.79 (d, 1H), 7.10 (d, 1H), 6.75 (s, 2H), 6.13 (d, 1H), 5.59 (t, 1H), 3.70 (m, 2H), 3.44 (m, 1H), 1.61 (quintet, 2H), 0.92 (t, 3H). MS (ES) for C₁₆H₁₈N₆O, found 299.2 (MH+).

### (S)-2-(3-(2-aminopyrimidin-4-yl)-1H-pyrrolo[2,3-b]pyridin-4-ylamino)butan-1-ol

¹H-NMR (400 MHz, DMSO-d₆): δ 11.8 (s, 1H), 10.2 (d, 1H), 8.08 (s, 1H), 8.07 (d, 1H), 7.79 (d, 1H), 7.10 (d, 1H), 6.75 (s, 2H), 6.13 (d, 1H), 5.59 (t, 1H), 3.70 (m, 2H), 3.44 (m, 1H), 1.61 (quintet, 2H), 0.92 (t, 3H). MS (ES) for C₁₆H₁₈N₆O, found 299.1 (MH+).

### 3-(2-aminopyrimidin-4-yl)-N-(pyrrolidin-3-ylmethyl)-1H-pyrrolo[2,3-b]pyridin-4-amine hydrochloride

¹H-NMR (400 MHz, DMSO-d₆): δ 13.4 (br s, 1H), 11.1 (s, 1H), 9.42 (s, 1H), 9.23 (s, 1H), 8.52 (s, 1H), 8.29 (d, 1H), 8.04 (d, 1H), 7.80 (br s, 1H), 7.43 (d, 1H), 6.79 (d, 1H), 3.77 (m, 2H), 3.24 (m, 2H), 3.08 (m, 1H), 2.85 (sextet, 1H), 2.61 (m, 1H), 2.02 (m, 1H), 1.63 (dq, 1H). MS (ES) for C₁₆H₁₉N₇, found 310.2 (MH+).

### 3-(2-aminopyrimidin-4-yl)-N-(pyrrolidin-2-ylmethyl)-1H-pyrrolo[2,3-b]pyridin-4-amine hydrochloride

¹H-NMR (400 MHz, DMSO-d₆): δ 13.4 (br s, 1H), 11.1 (s, 1H), 9.47 (s, 1H), 9.38 (s, 1H), 8.53 (s, 1H), 8.30 (d, 1H), 8.11 (d, 1H), 7.84 (br s, 1H), 7.43 (d, 1H), 6.85 (d, 1H), 4.12 (m, 1H), 3.98 (dt, 1H), 3.73 (m, 1H), 3.25 (m, 1H), 3.13 (m, 1H), 2.14 (m, 1H), 2.00 (m, 1H), 1.89 (m, 1H), 1.68 (dq, 1H). MS (ES) for C₁₆H₁₉N₇, found 310.2 (MH+).

### N¹-(3-(2-aminopyrimidin-4-yl)-1H-pyrrolo[2,3-b]pyridin-4-yl)-N³-methylpropane-1,3-diamine hydrochloride

¹H-NMR (400 MHz, DMSO-d₆): δ 13.4 (br s, 1H), 11.0 (s, 1H), 9.03 (s, 2H), 8.52 (s, 1H), 8.31 (d, 1H), 8.08 (d, 1H), 7.81 (br s, 1H), 7.44 (d, 1H), 6.75 (d, 1H), 3.78 (m, 2H), 2.95 (m, 2H), 2.52 (t, 3H), 2.00 (quintet, 2H). MS (ES) for C₁₆H₁₉N₇, found 298.1 (MH+).

### N¹-(3-(2-aminopyrimidin-4-yl)-1H-pyrrolo[2,3-b]pyridin-4-yl)cyclohexane-1,3-diamine hydrochloride (a 1:1 mixture of diastereomers).

¹H-NMR (400 MHz, DMSO-d₆): δ 13.2 (s, 1H), 10.4 (m, 1H), 8.36 (d, 1H), 8.33 (d, 1H), 8.19 (m, 3H), 8.09 (m, 1H), 7.34 (t, 1H), 7.21 (br s, 1H), 6.73 (m, 1H), 4.07 (m, 1H), 3.35 (m, 1H), 1.75 (m, 8H). MS (ES) for C₁₇H₂₁N₇, found 324.1 (MH+).

### (S)-3-(2-aminopyrimidin-4-yl)-N-(pyrrolidin-2-ylmethyl)-1H-pyrrolo[2,3-b]pyridin-4-amine hydrochloride

¹H-NMR (400 MHz, DMSO-d₆): δ 13.4 (br s, 1H), 11.0 (s, 1H), 9.51 (s, 1H), 9.42 (s, 1H), 8.55 (s, 1H), 8.31 (d, 1H), 8.12 (d, 1H), 7.85 (br s, 1H), 7.45 (d, 1H), 6.86 (d, 1H), 4.12 (m, 1H), 3.98 (dt, 1H), 3.73 (m, 1H), 3.25 (m, 1H), 3.13 (m, 1H), 2.14 (m, 1H), 2.00 (m, 1H), 1.89 (m, 1H), 1.68 (dq, 1H). MS (ES) for C₁₆H₁₉N₇, found 310.2 (MH+).

### (R)-3(2-aminopyrimidin-4-yl)-N-(pyrrolidin-2-ylmethyl)-1H-pyrrolo[2,3-b]pyridin-4-amine hydrochloride

¹H-NMR (400 MHz, DMSO-d₆): δ 13.4 (br s, 1H), 10.9 (s, 1H), 9.54 (s, 1H), 9.46 (s, 1H), 8.57 (s, 1H), 8.31 (d, 1H), 8.12 (d, 1H), 7.98 (br s, 1H), 7.46 (d, 1H), 6.87 (d, 1H), 4.14 (m, 1H), 3.99 (dt, 1H), 3.73 (m, 1H), 3.25 (m, 1H), 3.13 (m, 1H), 2.14 (m, 1H), 2.00 (m, 1H), 1.89 (m, 1H), 1.68 (dq, 1H). MS (ES) for C₁₆H₁₉N₇, found 310.2 (MH+).

### N²-(3-(2-aminopyrimidin-4-yl)-1H-pyrrolo[2,3-b]pyridin-4-yl)-N¹,N¹-dimethylpropane-1,2-diamine

¹H-NMR (400 MHz, DMSO-d₆): δ 11.8 (s, 1H), 9.69 (d, 1H), 8.09 (d, 1H), 8.02 (s, 1H), 7.83 (d, 1H), 7.06 (d, 1H), 6.74 (s, 2H), 6.18 (d, 1H), 3.75 (m, 1H), 2.64 (dd, 1H), 2.33 (dd, 1H), 2.19 (s, 6H), 1.20 (d, 3H). MS (ES) for C₁₆H₂₁N₇, found 312.2 (MH+).

### (S)-2-(3-(2-aminopyrimidin-4-yl)-1H-pyrrolo[2,3-b]pyridin-4-ylamino)-3-(dimethylamino)propan-1-ol

¹H-NMR (400 MHz, DMSO-d₆): δ 11.8 (s, 1H), 10.2 (d, 1H), 8.09 (s, 1H), 8.07 (d, 1H), 7.81 (d, 1H), 7.09 (d, 1H), 6.84 (s, 2H), 6.22 (d, 1H), 5.54 (br s, 1H), 3.80 (m, 1H), 3.65 (m, 2H), 2.55 (dd, 1H), 2.36 (dd, 1H), 2.22 (s, 6H). MS (ES) for C₁₆H₂₁N₇O, found 328.2 (MH+).

### N¹-(3-(2-aminopyrimidin-4-yl)-1H-pyrrolo[2,3-b]pyridin-4-yl)-N²-ethylethane-1,2-diamine hydrochloride

¹H-NMR (400 MHz, DMSO-d₆): δ 13.4 (br s, 1H), 10.9 (s, 1H), 9.25 (s, 2H), 8.54 (s, 1H), 8.31 (d, 1H), 8.13 (d, 1H), 7.92 (br s, 1H), 7.44 (d, 1H), 6.86 (d, 1H), 4.02 (q, 2H), 3.20 (m, 2H), 2.97 (sextet, 2H), 1.20 (t, 3H). MS (ES) for C₁₅H₁₉N₇, found 298.2 (MH+).

### Example 59

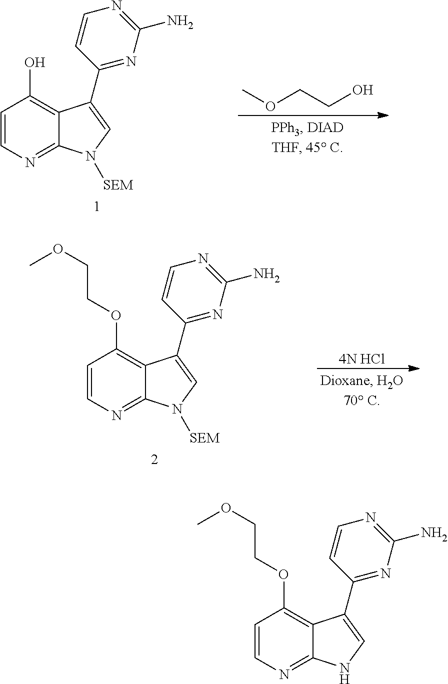

### 4-(4-(2-Methoxyethoxy)-1-((2-(trimethylsilyl)ethoxy)methyl)-1H-pyrrolo[2,3-b]pyridin-3-yl)pyrimidin-2-amine

To a mixture of 3-(2-aminopyrimidin-4-yl)-1-((2-(trimethylsilyl)ethoxy)methyl)-1H-pyrrolo[2,3-b]pyridin-4-ol 1 (80.0 mg, 0.224 mmol), methoxyethanol (25.5 mg, 0.336 mmol), triphenylphosphine (235 mg, 0.896 mmol) and THF (0.5 mL) at 45° C. was added diisopropyl azodicarboxylate (0.15 mL, 0.784 mmol). After stirring for 1 h, water (4 mL) was added and the resulting mixture was extracted with EtOAc (4×2 mL). The combined organic layers were concentrated and the resulting material was purified by silica gel column chromatography (Hexane/EtOAc=1:2→EtOAc/MeOH=30:1) to afford 4-(4-(2-methoxyethoxy)-14(2-(trimethylsilyl)ethoxy)methyl)-1H-pyrrolo[2,3-b]pyridin-3-yl)pyrimidin-2-amine 2 (39.0 mg, 42%) as a clear oil. MS (ES) for C₂₀H₂₉N₅O₃Si, found 416.2 (MH+).

Using analogous synthetic techniques as in Example 54, and substituting with the appropriate alternative starting materials, the following compounds were prepared. Alternative starting materials were obtained commercially unless otherwise indicated.

### 4-(4-(2-Methoxyethoxy)-1H-pyrrolo[2,3-b]pyridin-3-yl)pyrimidin-2-amine

The mixture of 4-(4-(2-methoxyethoxy)-14(2-(trimethylsilyl)ethoxy)methyl)-1H-pyrrolo[2,3-b ]pyridin-3-yl)pyrimidin-2-amine 2 (39.0 mg, 0.0938 mmol), HCl (1 mL, 4N in 1,4-dioxane) and aqueous HCl (1 mL, 4N) was stirred at 70° C. for 2 h. After removing all solvents, the resulting material was dissolved in ammonia (1 mL, 7N in MeOH) solution. The mixture was concentrated in vacuo and the resulting material was purified by silica gel chromatography (CH₂Cl₂/MeOH=30:1→20:1→CH₂Cl₂/7N NH₃ in MeOH=20:1) to give the title compound (19.0 mg, 71%) as a pale yellow powder. ¹H-NMR (400 MHz, DMSO-d₆): δ 12.1 (s, 1H), 8.15 (d, 1H), 8.14 (d, 1H), 7.94 (d, 1H), 7.47 (d, 1H), 6.77 (d, 1H), 6.32 (s, 2H), 4.32 (m, 2H), 3.77 (m, 2H), 3.37 (s, 3H). MS (ES) for C₁₄H₁₅N₅O₂, found 286.2 (MH+).

### 4-(4-(2-(dimethylamino)ethoxy)-1H-pyrrolo[2,3-b]pyridin-3-yl)pyrimidin-2-amine

¹H-NMR (400 MHz, DMSO-d₆): δ 12.0 (s, 1H), 8.15 (d, 1H), 8.14 (d, 1H), 7.93 (d, 1H), 7.52 (d, 1H), 6.79 (d, 1H), 6.31 (s, 2H), 4.27 (t, 2H), 2.73 (t, 2H), 2.24 (s, 6H). MS (ES) for C₁₅H₁₈N₆O, found 299.1 (MH+).

### 3-(3-(2-aminopyrimidin-4-yl)-1H-pyrrolo[2,3-b]pyridin-4-yloxy)propan-1-ol

¹H-NMR (400 MHz, DMSO-d₆): δ 12.1 (s, 1H), 8.15 (d, 1H), 8.14 (d, 1H), 7.90 (d, 1H), 7.31 (d, 1H), 6.78 (d, 1H), 6.34 (s, 2H), 4.62 (t, 1H), 4.27 (t, 2H), 3.59 (q, 2H), 1.98 (quintet, 2H). MS (ES) for C₁₄H₁₅N₅O₂, found 286.2 (MH+).

### 2-(3-(2-aminopyrimidin-4-yl)-1H-pyrrolo[2,3-b]pyridin-4-yloxy)ethanol

¹H-NMR (400 MHz, DMSO-d₆): δ 12.1 (s, 1H), 8.14 (d, 1H), 8.13 (d, 1H), 7.94 (s, 1H), 7.51 (d, 1H), 6.77 (d, 1H), 6.30 (s, 2H), 4.98 (br s, 1H), 4.24 (t, 2H), 3.85 (t, 2H). MS (ES) for C₁₃H₁₃N₅O₂, found 272.1 (MH+).

### Example 60

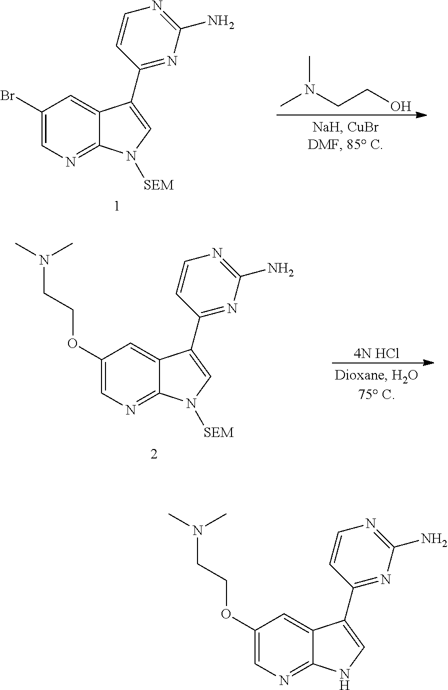

### 4-(5-(2-(dimethylamino)ethoxy)-1-((2-(trimethylsilyl)ethoxy)methyl)-1H-pyrrolo[2,3-b]pyridin-3-yl)pyrimidin-2-amine

To a stirred 2-dimethylaminoethanol (4.3 mL, 42.8 mmol) at 0° C. was added NaH (950 mg, 60% dispersion in mineral oil, 14.3 mmol). After 10 min, a mixture of 4-(5-bromo-14(2-(trimethylsilyl)ethoxy)methyl)-1H-pyrrolo[2,3-b]pyridin-3-yl)pyrimidin-2-amine 1 (120 mg, 0.285 mmol), CuBr (81.9 mg, 0.570 mmol) and DMF (5 mL) was added. After stirring for 1 h at 85° C., the reaction mixture was cooled down to room temperature. Water (5 mL) and the 9:1 mixture of aqueous saturated solution of NH₄C₁ and NH₄OH (5 mL) were added orderly, and the resulting mixture was extracted with the 9:1 mixture of EtOAc and MeOH (4×10 mL). The combined organic layers were concentrated and the resulting material was purified by silica gel column chromatography (CH₂Cl₂/MeOH=20:1→10:1→CH₂Cl₂/2N NH₃ in MeOH=20:1→15:1) to afford 4-(5-(2-(dimethylamino)ethoxy)-1-((2-(trimethylsilyl)ethoxy)methyl)-1H-pyrrolo[2,3-b]pyridin-3-yl)pyrimidin-2-amine 2 (49.0 mg, 40%) as a pale yellow foam. MS (ES) for C₂₁H₃₂N₆O₂Si, found 429.2 (MH+).

Using analogous synthetic techniques as in Example 54, and substituting with the appropriate alternative starting materials, the following compounds were prepared. Alternative starting materials were obtained commercially unless otherwise indicated.

### 4-(5-(2-(dimethylamino)ethoxy)-1H-pyrrolo[2,3-b]pyridin-3-yl)pyrimidin-2-amine

The mixture of 4-(5-(2-(dimethylamino)ethoxy)-14(2-(trimethylsilyl)ethoxy)methyl)-1H-pyrrolo[2,3-b]pyridin-3-yl)pyrimidin-2-amine 2 (49.0 mg, 0.114 mmol), HCl (1 mL, 4N in 1,4-dioxane) and aqueous HCl (1 mL, 4N) was stirred at 75° C. for 2 h. After removing all solvents, the resulting material was dissolved in ammonia (2 mL, 7N in MeOH) solution. The mixture was concentrated in vacuo and the resulting material was purified by silica gel chromatography (CH₂Cl₂/MeOH=10:1→CH₂Cl₂/7N NH₃ in MeOH=20:1) to give the title compound (26.0 mg, 76%) as a pale yellow powder. ¹H-NMR (400 MHz, DMSO-d₆): δ 12.1 (s, 1H), 8.15 (d, 1H), 8.14 (d, 1H), 7.93 (s, 1H), 7.52 (d, 1H), 6.79 (d, 1H), 6.31 (s, 2H), 4.27 (t, 2H), 2.73 (t, 2H), 2.24 (s, 6H). MS (ES) for C₁₅H₁₈N₆O, found 299.2 (MH+).

### 4-(5-methoxy-1H-pyrrolo[2,3-b]pyridin-3-yl)pyrimidin-2-amine

¹H-NMR (400 MHz, DMSO-d₆): δ 12.1 (s, 1H), 8.47 (d, 1H), 8.31 (d, 1H), 8.11 (d, 1H), 8.03 (d, 1H), 7.03 (d, 1H), 6.54 (s, 2H), 3.90 (s, 3H). MS (ES) for C₁₂H₁₁N₅O, found 242.1 (MH+).

### 4-(5-(2-methoxyethoxy)-1H-pyrrolo[2,3-b]pyridin-3-yl)pyrimidin-2-amine

¹H-NMR (400 MHz, DMSO-d₆): δ 12.1 (s, 1H), 8.47 (d, 1H), 8.31 (s, 1H), 8.11 (d, 1H), 8.04 (d, 1H), 7.03 (d, 1H), 6.55 (s, 2H), 4.24 (dd, 2H), 3.72 (dd, 2H), 3.36 (s, 3H). MS (ES) for C₁₄H₁₅N₅O₂, found 286.2 (MH+).

### Example 61

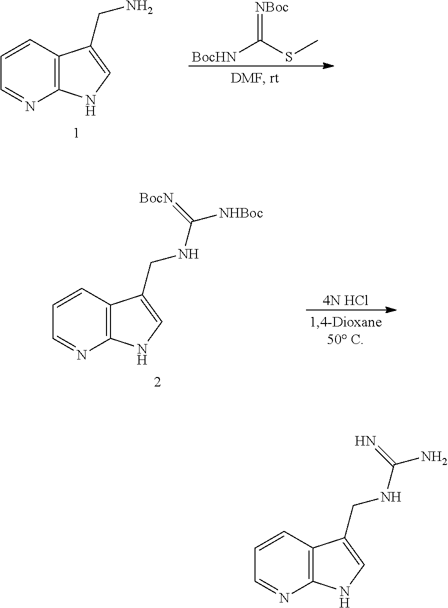

### tert-Butyl((1H-pyrrolo[2,3-b]pyridin-3-yl)methylamino)(tert-butoxycarbonylamino)methylene-carbamate

(1H-Pyrrolo[2,3-b]pyridin-3-yl)methanamine 1 was prepared by a literature method (Pedras, M. S. C.; Hossain, M. Bioorg. Med. Chem. 2007, 15, 5981-5996.) from 1H-pyrrolo[2,3-b]pyridine-3-carbaldehyde.

A mixture of (1H-pyrrolo[2,3-b]pyridin-3-yl)methanamine 1 (40.0 mg, 0.272 mmol), 1,3-bis(tert-butoxycarbonyl)-2-methyl-2-thiopseudourea (95.0 mg, 0.326 mmol) and DMF (2 mL) was stirred at room temperature for 1.5 h. Brine (4 mL) was added and the resulting mixture was extracted with CH₂Cl₂ (3×2 mL). The combined organic layers were concentrated and the resulting material was purified by silica gel column chromatography (Hexane/EtOAc=4:1→Hexane/EtOAc=3:2) to afford tert-butyl((1H-pyrrolo[2,3-b]pyridin-3-yl)methylamino)(tert-butoxycarbonylamino) methylenecarbamate 2 (32.0 mg, 30%) as a pale yellow solid. MS (ES) for C₁₉H₂₇N₆O₄, found 390.2 (MH+).

Using analogous synthetic techniques as in Example 54, and substituting with the appropriate alternative starting materials, the following compounds were prepared. Alternative starting materials were obtained commercially unless otherwise indicated.

### 1-((1H-Pyrrolo[2,3-b]pyridin-3-yl)methyl)quanidine hydrochloride

A mixture of tert-butyl ((1H-pyrrolo[2,3-b]pyridin-3-yl)methylamino)(tert-butoxycarbonylamino) methylenecarbamate 2 (32.0 mg, 0.0822 mmol) and HCl (1 mL, 4N in 1,4-dioxane) was stirred at 50° C. for 16 h. After removing all volatile materials, the resulting solid was washed by EtOAc (3×3 mL) to give the title compound (20.5 mg, quant) as a pale brown powder. ¹H-NMR (400 MHz, DMSO-d₆): δ 12.0 (s, 1H), 8.33 (dd, 1H), 8.23 (d, 1H), 8.04 (t, 1H), 7.61 (d, 1H), 7.50 (br s, 1H), 7.24 (dd, 1H), 7.05 (br s, 2H), 4.51 (d, 2H). MS (ES) for C₉H₁₁N₆, found 190.1 (MH+).

### Example 62

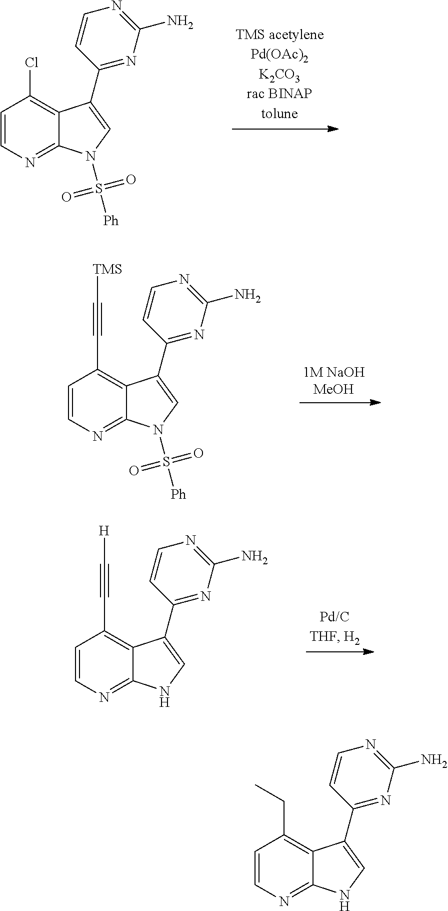

### 4-(1-(phenylsulfonyl)-4-((trimethylsilyl)ethynyl)-1H-pyrrolo[2,3-b]pyridin-3-yl)pyrimidin-2-amine

In an oven dried 3 neck round bottom flask equipped with a condenser and a stir bar was placed Pd(OAc)₂ (11.6 mg, 0.0517 mmol), rac BINAP (64.5 mg, 0.104 mmol) and K₂CO₃ (71.6 mg, 0.519 mmol). In a separate oven dried round bottom flask 4-(4-chloro-1-(phenylsulfonyl-1H-pyrrolo[2,3-b]pyridine-3-yl)pyrimidin-2-amine (100 mg, 0.260) was dissolved in 26 mL toluene this was transferred via syringe to the three neck flask, then the three neck flask was placed in a preheated heating mantle and allow to stir for 2 h at 100° C. The crude reaction was purified by silica gel flash chromatography followed by preparative HPLC to afford 4-(1-(phenylsulfonyl)-4-((trimethylsilyl)-ethynyl)-1H-pyrrolo[2,3-b]pyridin-3-yl)pyrimidin-2-amine (64 mg, 55%) as a white solid. ¹H-NMR (400 MHz, CDCl₃): δ 8.39 (d, 1H), 8.32 (d, 1H), 8.22 (m, 2H), 8.17 (s, 1H) 7.60 (m, 1H), 7.50 (m, 2H), 7.31 (d, 1H), 7.09 (d, 1H), 0.014 (s, 9H). MS (ES) for C₂₂H₂₁N₅O₂SSi, found 448.2 (MH+).

Using analogous synthetic techniques as in Example 54, and substituting with the appropriate alternative starting materials, the following compounds were prepared. Alternative starting materials were obtained commercially unless otherwise indicated.

### 4-(4-ethynyl-1H-pyrrolo[2,3-b]pyridin-3-yl)pyrimidin-2-amine

4-(1-(phenylsulfonyl)-4-((trimethylsilyl)ethynyl)-1H-pyrrolo[2,3-b]pyridin-3-yl)pyrimidin-2-amine (14 mg, 0.031 mmol) was dissolved in 4 mL methanol to this was added 2 mL 1 M NaOH. The reaction was allowed to stir for 30 minutes at room temperature at which time it was quenched with 1 mL acetic acid. The solution was concentrated in vacuo and purified by silica gel flash chromatography to afford 4-(4-ethynyl-1H-pyrrolo[2,3-b]pyridin-3-yl)pyrimidin-2-amine (7.0 mg, 95%) as a white solid. ¹H-NMR (400 MHz, CD₃OD): δ 8.25 (d, 1H), 8.21 (d, 1H), 7.97 (s, 1H), 7.32 (d, 1H), 7.16 (d, 1H), 4.05 (s, 1H), 2.00 (s, 1H). MS (ES) for C₁₃H₉N₅, found 236.1 (MH+).

### 4-(4-ethyl-1H-pyrrolo[2,3-b]pyridin-3-yl)pyrimidin-2-amine

4-(4-ethynyl-1H-pyrrolo[2,3-b]pyridin-3-yl)pyrimidin-2-amine (7.0 mg, 0.029 mmol) was dissolved in 3 mL THF to this was added Pd/C 10% by weight (15 mg). The reaction was allowed to stir under a balloon of hydrogen for 7 hours at room temperature. The crude reaction was filtered through celite with methanol and then concentrated in vacuo and purified by silica gel flash chromatography to afford 4-(4-ethyl-1H-pyrrolo[2,3-b]pyridin-3-yl)pyrimidin-2-amine (7 mg, 98%) as a white solid. ¹H-NMR (400 MHz, CD₃OD): δ 8.17 (d, 1H), 8.13 (d, 1H), 7.75 (s, 1H), 7.02 (d, 1H), 6.89 (d, 1H), 3.21 (q, 2H), 1.05 (t, 3H). MS (ES) for C₁₃H₁₃N₅, found 240.2 (MH+).

### 4-[4-(3-aminoprop-1-yn-1-yl)-1H-pyrrolo[2,3-b]pyridin-3-yl]pyrimidin-2-amine

¹H-NMR (400 MHz, CD₃OD): δ 8.49 (s, 1H), 8.42 (d, 1H), 8.28 (d, 1H), 7.57 (d, 1H), 7.47 (d, 1H), 4.15 (s, 2H). MS (ES) for C₁₄H₁₂N₆, found 265.1 (MH+).

### 4-{4-[3-(dimethylamino)prop-1-yn-1-yl]-1H-pyrrolo[2,3-b]pyridin-3-yl}pyrimidin-2-amine

¹H-NMR (400 MHz, CD₃OD): δ 8.24 (m, 2H), 7.92 (s, 1H), 7.26 (d, 1H), 7.07 (d, 1H), 3.54 (s, 2H), 2.30 (s, 6H), 1.94 (s, 6H). MS (ES) for C₁₆H₁₆N₆, found 393.2 (MH+).

### 4-{4-[3-(dimethylamino)propyl]-1H-pyrrolo[2,3-b]pyridin-3-yl}pyrimidin-2-amine

¹H-NMR (400 MHz, CD₃OD): δ 8.20 (d, 1H), 8.18 (d, 1H), 7.89 (s, 1H), 7.08 (d, 1H), 6.98 (d, 1H), 3.39 (t, 2H), 2.79 (t, 2H), 2.51 (s, 6H), 1.93 (s, 6H), 1.73 (m, 2H). MS (ES) for C₁₆H₂₀N₆, found 297.2 (MH+).

### 4-{4-[1-(phenylmethyl)-1H-1,2,3-triazol-4-yl]-1H-pyrrolo[2,3-b]pyridin-3-yl}pyrimidin-2-amine

¹H-NMR (400 MHz, CD₃OD): δ 8.35 (d, 1H), 7.91 (s, 1H), 7.80 (d, 1H), 7.77 (s, 1H), 7.41 (m, 6H), 6.29 (d, 1H), 5.57 (s, 2H), 1.93 (s, 3H). MS (ES) for C₂₀H₁₆N₈, found 369.2 (MH+).

### 3-[3-(2-aminopyrimidin-4-yl)-1H-pyrrolo[2,3-b]pyridin-4-yl]prop-2-yn-1-ol

¹H-NMR (400 MHz, CD₃OD): δ 8.24 (d, 1H), 8.22 (d, 1H), 7.97 (s, 1H), 7.26 (d, 1H), 7.17 (d, 1H), 4.40 (s, 2H), 1.93 (s, 1H). MS (ES) for C₁₄H₁₁N₅O, found 266.1 (MH+).

### 3-[3-(2-aminopyrimidin-4-yl)-1H-pyrrolo[2,3-b]pyridin-4-yl]propan-1-ol

¹H-NMR (400 MHz, CD₃OD): δ 8.19 (d, 1H), 8.15 (d, 1H), 7.82 (s, 1H), 7.05 (d, 1H), 6.93 (d, 1H), 3.45 (t, 2H), 1.93 (s, 3H), 1.67 (m, 2H). MS (ES) for C₁₄H₁₅N₅O, found 270.1 (MH+).

### 4-{4-[3-(methylamino)prop-1-yn-1-yl]-1H-pyrrolo[2,3-b]pyridin-3-yl}pyrimidin-2-amine

¹H-NMR (400 MHz, D₂O): δ 8.06 (d, 2H), 7.74 (s, 1H), 7.16 (d, 1H), 6.81 (d, 1H), 3.96 (s, 2H), 2.51 (s, 3H). MS (ES) for C₁₅H₁₄N₆, found 279.1 (MH+).

### 3-(2-aminopyrimidin-4-yl)-N-(2-pyridin-2-ylethyl)-1H-pyrrolo[2,3-b]pyridin-4-amine

¹H-NMR (400 MHz, CD₃OD): δ 8.47 (m, 1H), 8.03 (d, 1H), 7.88 (s, 1H), 7.83 (d, 1H), 7.68 (m, 1H), 7.32 (d, 1H), 7.23 (m, 1H), 7.03 (d, 1H), 6.34 (d, 1H), 3.77 (t, 2H), 3.21 (t, 2H). MS (ES) for C₁₈H₁₇N₇, found 332.1 (MH+).

### 3-(2-aminopyrimidin-4-yl)-N-(2-pyridin-4-ylethyl)-1H-pyrrolo[2,3-b]pyridin-4-amine

¹H-NMR (400 MHz, CD₃OD): δ 8.34 (d, 2H), 8.04 (d, 1H), 7.88 (d, 1H), 7.85 (d, 1H), 7.32 (d, 2H), 7.03 (d, 1H), 6.35 (d, 1H), 3.75 (t, 2H), 3.09 (t, 2H). MS (ES) for C₁₈H₁₇N₇, found 332.1 (MH+).

### N-[3-(2-aminopyrimidin-4-yl)-1H-pyrrolo[2,3-b]pyridin-4-yl]benzene-1,3-diamine

¹H-NMR (400 MHz, CD₃OD): δ 8.12 (d, 1H), 8.01 (s, 1H), 7.85 (d, 1H), 7.14 (m, 2H), 6.86 (d, 1H), 6.81 (m, 1H), 6.76 (m, 1H), 6.50 (m, 1H). MS (ES) for C₁₇H₁₅N₇, found 318.1 (MH+).

### N-[3-(2-aminopyrimidin-4-yl)-1H-pyrrolo[2,3-b]pyridin-4-yl]benzene-1,2-diamine

¹H-NMR (400 MHz, D₆-DMSO): δ 12.05 (s, 1H), 11.92 (s, 1H), 8.24 (s, 1H), 8.13 (d, 1H), 7.87 (d, 1H), 7.32 (d, 1H), 7.16 (d, 1H), 6.92 (m, 4H), 6.70 (m, 1H), 6.55 (d, 1H), 5.05 (s, 2H). MS (ES) for C₁₇H₁₅N₇, found 318.1 (MH+).

### N-[3-(2-aminopyrimidin-4-yl)-1H-pyrrolo[2,3-b]pyridin-4-yl]benzene-1,4-diamine

¹H-NMR (400 MHz, CD₃OD): δ 8.19 (d, 1H), 8.10 (s, 1H), 7.79 (d, 1H), 7.19 (m, 3H), 6.85 (m, 2H), 6.59 (d, 1H), 1.93 (s, 3H). MS (ES) for C₁₇H₁₅N₇, found 318.1 (MH+).

### 3-(2-aminopyrimidin-4-yl)-N-(2-piperidin-2-ylethyl)-1H-pyrrolo[2,3-b]pyridin-4-amine

¹H-NMR (400 MHz, CD₃OD): δ 8.22 (d, 1H), 8.20 (s, 1H), 7.97 (d, 1H), 7.25 (d, 1H), 6.69 (d, 1H), 3.77 (t, 2H), 2.98 (m, 2H), 2.16 (m, 2H), 2.04 (m, 2H), 1.90 (m, 2H), 1.54 (m, 3H). MS (ES) for C₁₈H₂₃N₇, found 338.1 (MH+).

### Example 63

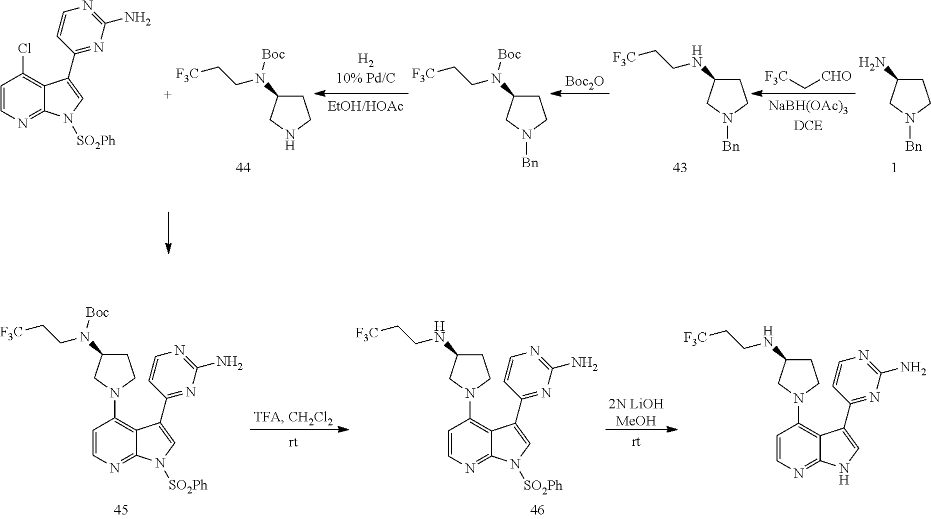

### (S)-1-Benzyl-N-(3,3,3-trifluoropropyl)pyrrolidin-3-amine (43)

(S)-1-Benzylpyrrolidin-3-amine (726 mg, 4.11 mmol) was treated with 3,3,3-trifluoropropanal (346 mg, 4.06 mmol) and NaBH(OAc)₃ (1.33 g, 6.3 mmol) in DCE (10 mL) at rt for 18 h. The mixture was pured onto saturated aq NaHCO₃ (50 mL) and extracted with CHCl₃ (2×50 mL). The combined organic layers were dried over MgSO₄. After purification by flash chromatography (92:7:1 dichloromethane/methanol/28% (w/w) ammonium hydroxide), the title compound was obtained as an oil (751 mg, 68% yield).

¹H-NMR (400 MHz, CDCl₃): δ 7.30 (m, 5H), 3.60 (d, 2H), 3.25 (m, 1H), 2.79 (m, 2H), 2.67 (m, 2H), 2.45 (m, 1H), 2.37 (m, 1H), 2.29 (m, 2H), 2.07 (m, 1H), 1.55 (m, 1H). ¹³C-NMR (100 MHz, CDCl₃): δ 139.2, 129.0, 128.5, 127.2, 122.7-131.0 (q), 60.7, 60.6, 57.6, 53.2, 41.2 (q), 34.8 (q), 32.3. ¹⁹F-NMR (376 MHz, CDCl₃): 6-65.5 (t, 3F). MS (ES) for C₁₄H₁₉F₃N₂, found 273 (MH+).

### (S)-tert-Butyl pyrrolidin-3-yl(3,3,3-trifluoropropyl)carbamate (44)

(S)-1-Senzyl-N-(3,3,3-trifluoropropyl)pyrrolidin-3-amine (43) was treated in a manner similar to Example 45, with (43) instead of ((3R,4R)-benzyl 3-amino-4-hydroxypyrrolidine-1-carboxylate (7) as substrate. The title compound was synthesized in a manner similar to Example 46, with the obtained intermediate, which was not fully characterized, instead of (S)-tert-butyl 1-benzylpyrrolidin-3-yl(2-(tert-butyldimethylsilyloxy)ethyl)carbamate (2) as substrate.

¹H-NMR (400 MHz, CDCl₃): δ 4.28 (m, 1H), 3.41 (m, 2H), 3.10 (m, 2H), 2.84 (m, 2H), 2.38 (m, 3H), 2.06 (m, 1H), 1.73 (m, 1H), 1.47 (s, 9H). ¹⁹F-NMR (376 MHz, CDCl₃): δ −66.0 (t, 3F). MS (ES) for C₁₂H₂₁F₃N₂O₂, found 283 (MH+).

### (S)-tert-Butyl 1-(3-(2-aminopyrimidin-4-yl)-1-(phenylsulfonyl)-1 l-pyrrolo[2,3-b]pyridin-4-yl)pyrrolidin-3-yl(3,3,3-trifluoropropyl)carbamate (45)

The title compound was obtained by S_{N}Ar of 4-(4-chloro-1-(phenylsulfonyl)-1H-pyrrolo[2,3-b]pyridin-3-yl)pyrimidin-2-amine (114 mg, 0.295 mmol) with (S)-tert-butyl pyrrolidin-3-yl(3,3,3-trifluoropropyl)carbamate (44, 137 mg, 0.486 mmol) and DIEA in n-PrOH at 110° C. for 16 h, followed by purification by flash chromatography (91:8:1 dichloromethane/methanol/28% (w/w) ammonium hydroxide), as a solid (155 mg, 83% yield).

¹H-NMR (400 MHz, CDCl₃): δ 8.31 (d, 1H), 8.23 (d, 2H), 8.17 (m, 1H), 7.59 (m, 1H), 7.49 (m, 2H), 6.86 (d, 1H), 6.50 (d, 1H), 5.39 (s, 2H), 3.28 (m, 4H), 3.09 (m, 1H), 2.99 (m, 1H), 2.27 (m, 2H), 1.88 (m, 1H), 1.48 (s, 9H). ¹⁹F-NMR (376 MHz, CDCl₃): δ −65.4 (t, 3F). MS (ES) for C₂₉H₃₂F₃N₇O₄S, found 632 (MH+).

### (S)-4-(1-(Phenylsulfonyl)-4-(3-(3,3,3-trifluoropropylamino)pyrrolidin-1-yl)-1H-pyrrolo[2,3-b]pyridin-3-yl)pyrimidin-2-amine (46)

(S)-tert-Butyl 1-(3-(2-aminopyrimidin-4-yl)-1-(phenylsulfonyl)-1l-pyrrolo[2,3-b]pyridin-4-yl)pyrrolidin-3-yl(3,3,3-trifluoropropyl)carbamate (45, 155 mg, 0.245 mmol) was treated with TFA (5 mL) in CH₂Cl₂ (5 mL) for 2 h at rt. The mixture was concentrated and azeotroped with toluene.

After purification by flash chromatography (92:7:1 dichloromethane/methanol/28% (w/w) ammonium hydroxide), the title compound was obtained as a solid (124 mg, 95% yield).

¹H-NMR (400 MHz, CDCl₃): δ 8.25 (d, 1H), 8.22 (d, 2H), 8.15 (m, 1H), 7.57 (m, 1H), 7.50 (m, 2H), 6.86 (d, 1H), 6.49 (d, 1H), 5.39 (s, 2H), 3.25 (m, 3H), 3.08 (m, 1H), 2.73 (m, 2H), 2.01 (m, 1H), 1.62 (m, 1H). ¹³C-NMR (100 MHz, CDCl₃): δ 161.0, 156.4, 149.1, 147.7, 144.0, 136.0, 132.3, 127.0, 126.5, 122.4, 117.5, 108.6, 106.9, 102.9, 55.1, 47.7, 38.9, 33.0, 32.5, 29.6. ¹⁹F-NMR (376 MHz, CDCl₃): δ −65.4 (t, 3F). MS (ES) for C₂₄H₂₄F₃N₇O₂S, found 532 (MH+).

### 4-(4-{(3S)-3-[(3,3,3-Trifluoropropyl)amino]pyrrolidin-1-yl}-1H-pyrrolo[2,3-b]pyridin-3-yl)pyrimidin-2-amine

(S)-4-(1-(phenylsulfonyl)-4-(3-(3,3,3-trifluoropropylamino)pyrrolidin-1-yl)-1H-pyrrolo[2,3-b]pyridin-3-yl)pyrimidin-2-amine (46, 225 mg, 0.423 mmol) was treated with 2 N LiOH (1 mL) in methanol for 16 h at rt. The mixture was concentrated, azeotroped with acetonitrile, then loaded on a silica gel column. After purification by flash chromatography (91:8:1 dichloromethane/methanol/28% (w/w) ammonium hydroxide), the title compound was obtained as a solid (83 mg, 50% yield).

¹H-NMR (400 MHz, CDCl₃): δ 12.8 (br s, 1H), 8.20 (d, 1H), 8.02 (d, 1H), 6.84 (d, 1H), 6.39 (d, 1H), 6.00 (m, 2H), 3.38 (m, 1H), 3.30 (m, 2H), 3.13 (m, 1H), 2.96 (dd, 1H), 2.76 (m, 2H), 2.05 (m, 1H), 1.63 (m, 1H). ¹³C-NMR (100 MHz, CDCl₃): δ 164.3, 163.5, 157.7, 151.8, 151.5, 143.9, 128.2, 126.0, 125.4, 115.5, 110.3, 107.8, 102.8, 57.3, 57.0, 49.9, 41.1, 34.9, 34.6, 31.9. ¹⁹F-NMR (376 MHz, CDCl₃): δ −65.4 (t, 3F). MS (ES) for C₁₈H₂₀F₃N₇, found 392 (MH+).

### Example 64

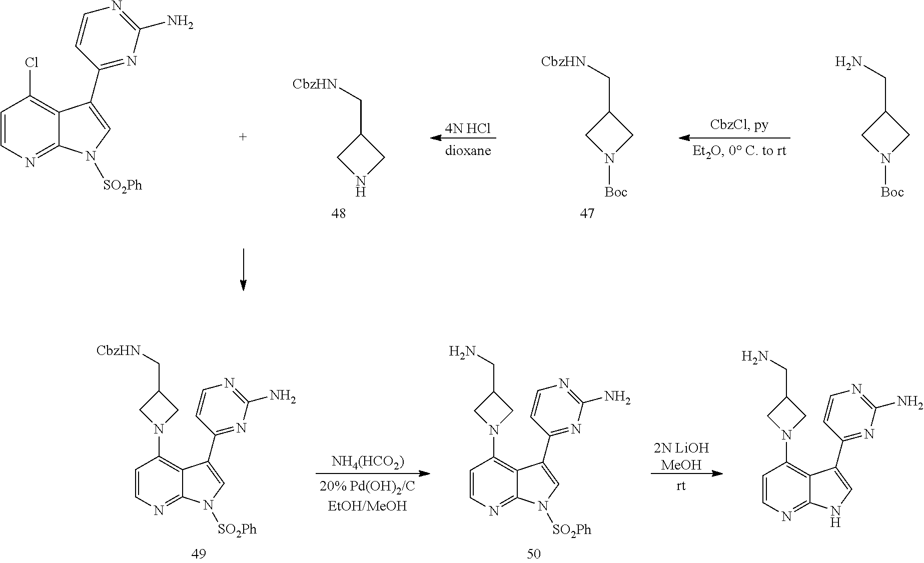

### tert-Butyl 3-((benzyloxycarbonylamino)methyl)azetidine-1-carboxylate (47)

tert-Butyl 3-(aminomethyl)azetidine-1-carboxylate (1.00 g, 5.37 mmol) was treated with pyridine (1.0 mL) and CbzCl (1.0 mL, 7.0 mmol) in Et₂O (15 mL) at 0° C. The mixture was stirred at rt for 18 h, then poured onto water. The organic phase was diluted with Et₂O, then washed with 0.5 N NaHSO₄, water, saturated aq NaHCO₃, brine, and dried over MgSO₄. After purification by flash chromatography (7:3:0.5 hexanes/ethyl acetate/methanol), the title compound was obtained as an oil (1.082 g, 63% yield).

¹H-NMR (400 MHz, CDCl₃): δ 7.34 (m, 5H), 5.09 (s, 2H), 3.96 (m, 2H), 3.59 (m, 2H), 3.37 (m, 2H), 2.69 (m, 1H), 1.41 (s, 9H). MS (ES) for C₁₇H₂₄N₂O₄, found 321 (MH+).

### Benzyl azetidin-3-ylmethylcarbamate (48)

tert-Butyl 3-((benzyloxycarbonylamino)methyl)azetidine-1-carboxylate (47, 553 mg, 1.73 mmol) was treated with 4N HCl/anhydrous dioxane (3 mL) in EtOAc (15 mL) at rt for 5 h. After concentration to dryness, the mixture was dissolved in MeOH and stirred with basic ion exchange Bio-Rad® AG 1-x8 resin (200 mg, hydroxide form) for 10 min, until a basic pH was reached, finally filtered through fritted septum. After removal of volatiles in vacuo the title compound was obtained as an oil (192 mg, 50% yield).

¹H-NMR (400 MHz, CDCl₃): δ 7.33 (m, 5H), 5.09 (s, 2H), 3.61 (m, 2H), 3.29 (m, 2H), 3.16 (m, 1H), 2.78 (m, 2H). MS (ES) for C₁₂H₁₆N₂O₂, found 221 (MH+).

### 4-(4-(3-(Aminomethyl)azetidin-1-yl)-1-(phenylsulfonyl)-1H-pyrrolo[2,3-b]pyridin-3-yl)pyrimidin-2-amine (50)

Benzyl (1-(3-(2-aminopyrimidin-4-yl)-1-(phenylsulfonyl)-1H-pyrrolo[2,3-b]pyridin-4-yl)azetidin-3-yl)methylcarbamate (49) was obtained by S_{N}Ar of 4-(4-chloro-1-(phenylsulfonyl)-1H-pyrrolo[2,3-b]pyridin-3-yl)pyrimidin-2-amine (118 mg, 0.306 mmol) with benzyl azetidin-3-ylmethylcarbamate (48, 192 mg, 0.873 mmol) and DIEA in n-PrOH at 110° C. for 16 h. The title compound was synthesized in a manner similar to Example 3 with intermediate 49 instead of (3R,4R)-benzyl 3-(tert-butoxycarbonylamino)-4-hydroxypyrrolidine-1-carboxylate (8) as substrate.

¹H-NMR (400 MHz, CD₃OD): δ 8.30 (m, 1H), 8.18 (m, 2H), 8.10 (m, 1H), 7.85 (s, 1H), 7.63 (m, 1H), 7.54 (m, 2H), 6.95 (m, 1H), 6.34 (m, 1H), 3.86 (m, 2H), 3.44 (m, 2H), 2.81 (m, 2H), 2.60 (m, 1H). MS (ES) for C₂₁H₂₁N₇O₂S, found 436 (MH+).

### 4-{4-[3-(Aminomethyl)azetidin-1-yl]-1H-pyrrolo[2,3-b]pyridin-3-yl}pyrimidin-2-amine

The title compound was synthesized in a manner similar as above with 4-(4-(3-(aminomethyl)azetidin-1-yl)-1-(phenylsulfonyl)-1H-pyrrolo[2,3-b]pyridin-3-yl)pyrimidin-2-amine (50) instead of (S)-4-(1-(phenylsulfonyl)-4-(3-(3,3,3-trifluoropropylamino)pyrrolidin-1-yl)-1H-pyrrolo[2,3-b]pyridin-3-yl)pyrimidin-2-amine (46) as substrate.

¹H-NMR (400 MHz, CD₃OD): δ 8.20 (d, 1H), 7.98 (d, 1H), 7.56 (s, 1H), 6.93 (d, 1H), 6.31 (m, 1H), 3.91 (m, 2H), 3.51 (m, 2H), 2.83 (m, 2H), 2.61 (m, 1H). MS (ES) for C₁₅H₁₇N₇, found 296 (MH+).

### Example 65

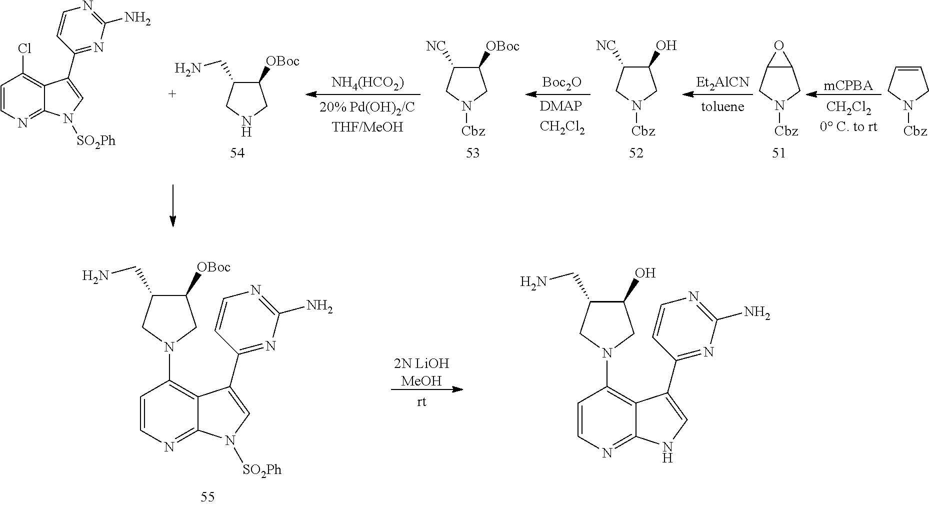

Compound 51 was made following the known procedure as referenced in Davis, B. G. et al Org Lett 2002, 4, 103.

Compound 52 was made following the known procedure as referenced in Benedetti, F. et al Tetrahedron Lett 1999, 40, 1041.

### Benzyl 3-(tert-butoxycarbonyloxy)-4-cyanopyrrolidine-1-carboxylate (53)

Benzyl 3-cyano-4-hydroxypyrrolidine-1-carboxylate (52, 309 mg, 1.25 mmol) was treated with Boc₂O (480 mg, 2.20 mmol) and DMAP (16 mg, 0.13 mmol) in CH₂Cl₂ (20 mL) at rt for 16 h. After concentration under reduced pressure, the mixture was purified by flash chromatography (8:2 hexanes/ethyl acetate), to afford the title compound as an oil (364 mg, 84% yield).

¹H-NMR (400 MHz, CDCl₃): δ 7.34 (m, 5H), 5.27 (m, 1H), 5.14 (s, 2H), 3.85 (m, 3H), 3.64 (m, 1H), 3.27 (m, 1H), 1.41 (s, 9H). MS (ES) for C₁₈H₂₂N₂O₅, found 347 (MH+).

### 4-(Aminomethyl)pyrrolidin-3-yl tert-butyl carbonate (±54)

The title compound was synthesized in a manner similar to Example 45 with benzyl 3-(tert-butoxycarbonyloxy)-4-cyanopyrrolidine-1-carboxylate (53) instead of (3R,4R)-benzyl 3-(tert-butoxycarbonylamino)-4-hydroxypyrrolidine-1-carboxylate (8) as substrate.

¹H-NMR (400 MHz, CDCl₃): δ 4.81 (m, 1H), 3.31 (m, 1H), 3.09 (m, 1H), 2.99 (dd, 1H), 2.81 (dd, 2H), 2.68 (dd, 1H), 2.55 (dd, 1H), 2.19 (m, 1H), 1.40 (s, 9H). ¹³C-NMR (100 MHz, CDCl₃): δ 153.4, 82.6, 82.1, 23.3, 50.7, 50.2, 44.1, 28.0. MS (ES) for C₁₀H₂₀N₂O₃, found 217 (MH+).

### 2-(Aminomethyl)-4-(3-(2-aminopyrimidin-4-yl)-1-(phenylsulfonyl)-1H-pyrrolo[2,3-b]pyridin-4-yl)cyclopentyl tert-butyl carbonate (55)

The title compound was obtained by S_{N}Ar of 4-(4-chloro-1-(phenylsulfonyl)-1H-pyrrolo[2,3-b]pyridin-3-yl)pyrimidin-2-amine (100 mg, 0.259 mmol) with 4-(aminomethyl)pyrrolidin-3-yl tert-butyl carbonate (±54, 83 mg, 0.384 mmol) and DIEA in n-PrOH at 110° C. for 16 h, followed by purification by flash chromatography (91:8:1 dichloromethane/methanol/28% (w/w) ammonium hydroxide), as a solid (58 mg, 40% yield).

¹H-NMR (400 MHz, CDCl₃): δ 8.24 (d, 1H), 8.19 (m, 3H), 7.97 (s, 1H), 7.57 (m, 1H), 7.50 (m, 2H), 6.94 (d, 1H), 6.54 (d, 1H), 5.29 (m, 2H), 3.61 (m, 1H), 3.59 (m, 1H), 3.42 (m, 2H), 3.22 (m, 1H), 2.87 (m, 1H), 2.72 (m, 1H), 2.61 (m, 1H), 2.22 (m, 1H), 1.47 (s, 9H). ¹³C-NMR (100 MHz, CDCl₃): δ 163.2, 162.5, 158.9, 153.1, 150.9, 146.2, 138.1, 134.4, 129.2, 128.6, 125.3, 119.6, 110.5, 109.7, 105.8, 83.0, 55.7, 53.1, 47.2, 43.2, 27.9. MS (ES) for C₂₇H₃₁N₇O₅S, found 566 (MH+).

### (3R,4S)-4-(Aminomethyl)-1-[3-(2-aminopyrimidin-4-O-1H-pyrrolo[2,3-b]pyridin-4-yl]pyrrolidin-3-ol

The title compound was synthesized in a manner similar to that above, with 2-(aminomethyl)-4-(3-(2-aminopyrimidin-4-yl)-1-(phenylsulfonyl)-1H-pyrrolo[2,3-b]pyridin-4-yl)cyclopentyl tert-butyl carbonate (55) instead of (S)-4-(1-(phenylsulfonyl)-4-(3-(3,3,3-trifluoropropylamino)pyrrolidin-1-yl)-1H-pyrrolo[2,3-b]pyridin-3-yl)pyrimidin-2-amine (46) as substrate.

¹H-NMR (400 MHz, CD₃OD): δ 8.19 (d, 1H), 8.00 (d, 1H), 7.87 (m, 1H), 7.60 (m, 1H), 7.41 (m, 1H), 6.93 (d, 1H), 6.53 (d, 1H), 3.59 (m, 1H), 3.48 (m, 1H), 3.35 (m, 1H), 3.10 (m, 2H), 2.86 (m, 1H), 2.78 (m, 1H), 2.28 (m, 1H). MS (ES) for C₁₆H₁₉N₇O, found 326 (MH+).

### Example 66

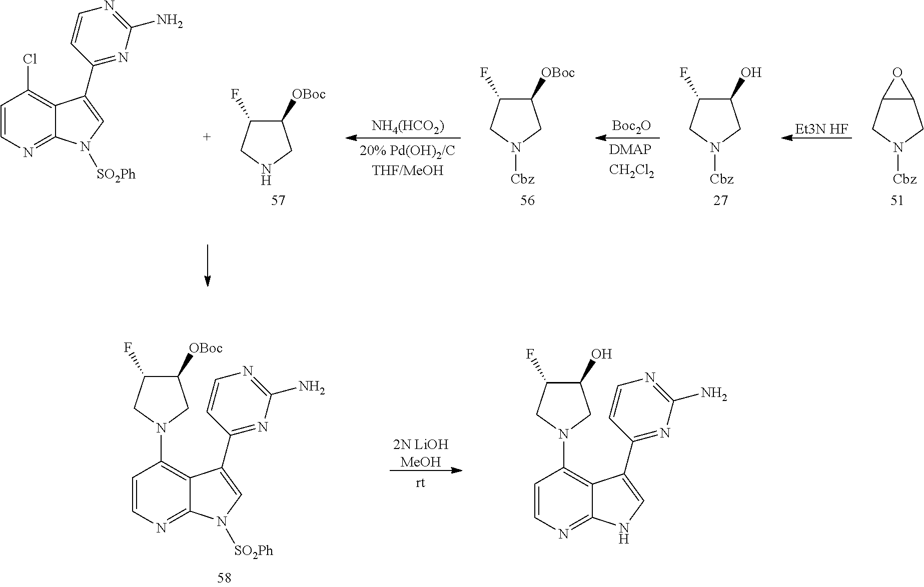

### (±)-(3S,4S)-benzyl 3-(tert-butoxycarbonyloxy)-4-fluoropyrrolidine-1-carboxylate (56)

The title compound was prepared in a manner similar to Example 65, with (3S,4S)-benzyl 3-fluoro-4-hydroxypyrrolidine-1-carboxylate instead of benzyl 3-cyano-4-hydroxypyrrolidine-1-carboxylate (52) as substrate.

Compound 27 was synthesized according to the procedure described in Example 51.

¹H-NMR (400 MHz, CDCl₃): δ 7.36 (m, 5H), 5.13 (s, 2H), 5.05 (m, 1H), 3.76 (m, 5H), 1.49 (s, 9H). ¹³C-NMR (100 MHz, CDCl₃): δ 154.8, 152.2, 136.7, 128.7, 128.3, 128.2, 91.8 (d), 83.9, 76.3 (d), 49.9 (d), 27.9. MS (ES) for C₁₇H₂₂FNO₅, found 340 (MH+).

### tert-Butyl-4-fluoropyrrolidin-34 carbonate (±57)

The title compound was synthesized in a manner similar to Example 65 with (±)-(3S,4S)-benzyl 3-(tert-butoxycarbonyloxy)-4-fluoropyrrolidine-1-carboxylate (56) instead of (3R,4R)-benzyl 3-(tert-butoxycarbonylamino)-4-hydroxypyrrolidine-1-carboxylate (8) as substrate, and directly used in the following step without further purification.

### (±)-1-(3-(2-Aminopyrimidin-4-yl)-1-(phenylsulfonyl)-1H-pyrrolo[2,3-b]pyridin-4-yl)-4-fluoropyrrolidin-3-yl tert-butyl carbonate (58)

The title compound was obtained by S_{N}Ar of 4-(4-chloro-1-(phenylsulfonyl)-1H-pyrrolo[2,3-b]pyridin-3-yl)pyrimidin-2-amine (129 mg, 0.334 mmol) with tert-butyl-4-fluoropyrrolidin-3-yl carbonate (±57, 120 mg, 0.509 mmol) and DIEA in n-PrOH at 110° C. for 16 h, followed by purification by flash chromatography (91:8:1 dichloromethane/methanol/28% (w/w) ammonium hydroxide), as a solid (63 mg, 34% yield).

¹H-NMR (400 MHz, CD₃OD): δ 8.22 (d, 1H), 8.15 (m, 2H), 8.01 (d, 1H), 7.63 (m, 1H), 7.54 (m, 2H), 6.88 (d, 1H), 6.63 (d, 1H), 4.21 (m, 2H), 3.54 (m, 1H), 3.30 (m, 2H), 3.05 (m, 1H). MS (ES) for C₂₆H₂₇FN₆O₅S, found 315 (MH+).

### (3S,4S)-1-[3-(2-aminopyrimidin-4-yl)-1H-pyrrolo[2,3-b]pyridin-4-yl]-4-fluoropyrrolidin-3-ol

The title compound was synthesized in a manner similar to that above, with (±)-1-(3-(2-aminopyrimidin-4-yl)-1-(phenylsulfonyl)-1H-pyrrolo[2,3-b]pyridin-4-yl)-4-fluoropyrrolidin-3-yl tert-butyl carbonate (58) instead of (S)-4-(1-(phenylsulfonyl)-4-(3-(3,3,3-trifluoropropylamino)pyrrolidin-1-yl)-1H-pyrrolo[2,3-b]pyridin-3-yl)pyrimidin-2-amine (46) as substrate.

¹H-NMR (400 MHz, CD₃OD): δ 8.14 (d, 1H), 7.97 (d, 1H), 7.61 (s, 1H), 6.91 (d, 1H), 6.57 (d, 1H), 4.25 (m, 1H), 3.62 (m, 1H), 3.58 (m, 1H), 3.45 (m, 2H), 3.16 (m, 1H). ¹⁹F-NMR (376 MHz, CD₃OD): 6-184.1 (m, 1F). MS (ES) for C₁₅H₁₅FN₆O, found 315 (MH+).

### Example 67

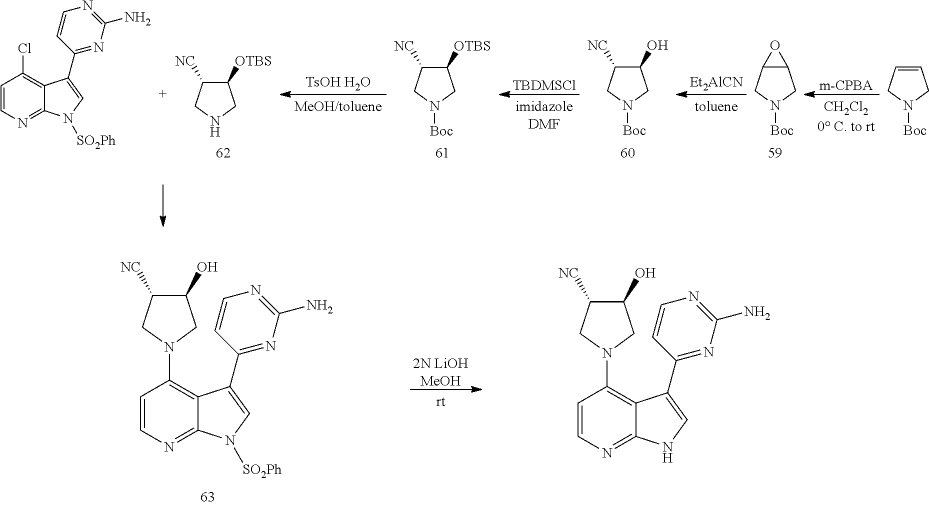

### tert-Butyl 6-oxa-3-azabicyclo[3.1.0]hexane-3-carboxylate (59)

The title compound was made following the known procedure as referenced in Davis, B. G. et al Org Lett 2002, 4, 103.

¹H-NMR (400 MHz, CDCl₃): δ 3.81 (d, 1H), 3.73 (d, 1H), 3.67 (m, 2H), 3.32 (m, 2H), 1.44 (s, 9H). ¹³C-NMR (100 MHz, CDCl₃): δ 155.0, 80.0, 55.8, 55.3, 47.5, 47.1, 28.6.

### (±)-(3R,4R)-tert-Butyl 3-cyano-4-hydroxypyrrolidine-1-carboxylate (60)

The title compound was prepared synthesized in a manner similar to Example 65, with tert-butyl 6-oxa-3-azabicyclo[3.1.0]hexane-3-carboxylate (59) instead of benzyl 6-oxa-3-azabicyclo[3.1.0]hexane-3-carboxylate (51) as substrate, and directly utilized in the next step without further purification.

### (±)-(3R,4R)-tert-Butyl 3-(tert-butyldimethylsilyloxy)-4-cyanopyrrolidine-1-carboxylate (61)

(3R,4R)-tert-Butyl 3-cyano-4-hydroxypyrrolidine-1-carboxylate (60, 239 mg, 1.12 mmol) was treated with TBDMSCI (197 mg, 1.31 mmol) and imidazole (173 mg, 2.54 mmol) in DMF at rt for 16 h. Upon completion of reaction, the mixture was poured onto water and extracted with ethyl acetate (2×50 mL). Combined organic layers were washed with water (4×30 mL) and brine, finally dried over MgSO₄. After purification by flash chromatography (8:2 hexanes/ethyl acetate), the title compound was obtained as an oil (234 mg, 64% yield).

¹H-NMR (400 MHz, CDCl₃): δ 4.42 (m, 1H), 3.51-3.68 (m, 3H), 3.14 (m, 1H), 2.86 (m, 1H), 1.38 (s, 9H), 0.82 (s, 9H), 0.03 (s, 6H). MS (ES) for C₁₆H₃₀N₂O₃Si, found 327 (MH+).

### (±)-(3R,4R)-1-(3-(2-aminopyrimidin-4-O-1-(phenylsulfonyl)-1H-pyrrolo[2,3-b]pyridin-4-yl)-4-hydroxypyrrolidine-3-carbonitrile (63)

(3R,4R)-tert-Butyl 3-(tert-butyldimethylsilyloxy)-4-cyanopyrrolidine-1-carboxylate (±61, 234 mg, 0.717 mmol) was treated with TsOH.H₂O (360 mg, 1.89 mmol) in MeOH (10 mL) and toluene (10 mL) at rt for 16 h, then at 55° C. for 3 h. Upon completion of the reaction, the mixture was brought to dryness under reduced pressure, then diluted with methanol and treated with Bio-Rad® AG 1-x8 resin (100 mg, hydroxide form) for 10 min, until a basic pH was reached, finally filtered through fritted septum. After removal of volatiles and purification by flash chromatography (91:8:1 dichloromethane/methanol/28% (w/w) ammonium hydroxide), compound±62 was obtained as as an oil (61 mg, 38% yield) and directly used for the next step without further characterization.

The title compound was obtained by S_{N}Ar of 4-(4-chloro-1-(phenylsulfonyl)-1H-pyrrolo[2,3-b]pyridin-3-yl)pyrimidin-2-amine (69 mg, 0.179 mmol) with (3R,4R)-4-(tert-butyldimethylsilyloxy)pyrrolidine-3-carbonitrile (±62, 61 mg, 0.269 mmol) and DIEA in n-PrOH at 110° C. for 16 h, followed by purification by flash chromatography (91:8:1 dichloromethane/methanol/28% (w/w) ammonium hydroxide), as a solid (18 mg, 22% yield).

¹H-NMR (400 MHz, CD₃OD): δ 8.13 (d, 1H), 8.02 (m, 2H), 7.95 (d, 1H), 7.91 (s, 1H), 7.51 (m, 1H), 7.41 (m, 2H), 6.80 (d, 1H), 6.58 (d, 1H), 4.27 (m, 1H), 3.47 (m, 1H), 3.30 (m, 2H), 2.94 (m, 1H), 2.81 (m, 1H). MS (ES) for C₂₂H₁₉N₇O₃S, found 462 (MH+).

### (3R,4R)-1-[3-(2-aminopyrimidin-4-yl)-1H-pyrrolo[2,3-b]pyridin-4-yl]-4-hydroxypyrrolidine-3-carbonitrile

The title compound was synthesized in a manner similar to that above, with (±)-(3R,4R)-1-(3-(2-aminopyrimidin-4-yl)-1-(phenylsulfonyl)-1H-pyrrolo[2,3-b]pyridin-4-yl)-4-hydroxypyrrolidine-3-carbonitrile (63) instead of (S)-4-(1-(phenylsulfonyl)-4-(3-(3,3,3-trifluoropropylamino)pyrrolidin-1-yl)-1 H-pyrrolo[2,3-b]pyridin-3-yl)pyrimidin-2-amine (46) as substrate.

¹H-NMR (400 MHz, CD₃OD): δ 8.19 (d, 1H), 8.04 (d, 1H), 7.72 (s, 1H), 7.00 (d, 1H), 6.67 (d, 1H), 4.47 (m, 1H), 3.68 (m, 2H), 3.50 (m, 1H), 3.13 (m, 2H), 2.98 (m, 1H). MS (ES) for C₁₆H₁₅N₇O, found 323 (MH+).

### Example 68

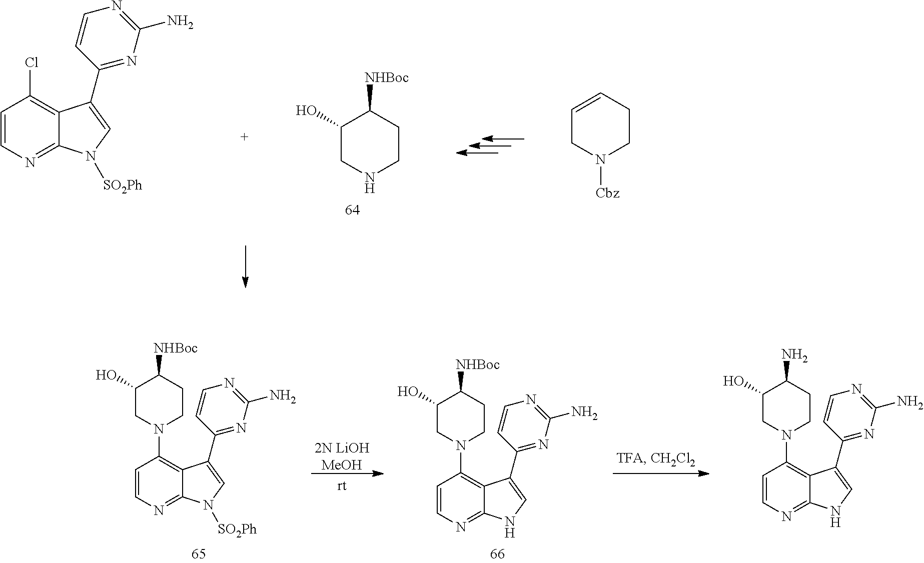

### (±)-tert-Butyl (3S,4S)-3-hydroxypiperidin-4-ylcarbamate (64)

The title compound was made following the known procedure as referenced in WO2005/066176.

### (±)-tert-butyl (3S,4S)-1-(3-(2-aminopyrimidin-4-yl)-1-(phenylsulfonyl)-1H-pyrrolo[2,3-b]pyridin-4-yl)-3-hydroxypiperidin-4-ylcarbamate (65)

The title compound was obtained by S_{N}Ar of 4-(4-chloro-1-(phenylsulfonyl)-1H-pyrrolo[2,3-b]pyridin-3-yl)pyrimidin-2-amine (160 mg, 0.415 mmol) with tert-butyl (3S,4S)-3-hydroxypiperidin-4-ylcarbamate (±64, 166 mg, 0.767 mmol) and DIEA in n-PrOH at 110° C. for 16 h, followed by purification by flash chromatography (91:8:1 dichloromethane/methanol/28% (w/w) ammonium hydroxide), as a solid (146 mg, 62% yield).

¹H-NMR (400 MHz, CDCl₃): δ 8.31 (d, 1H), 8.25 (m, 1H), 7.68 (m, 1H), 7.55 (m, 4H), 6.93 (d, 1H), 6.70 (d, 1H), 5.46 (br s, 2H), 5.00 (br s, 1H), 4.38 (br s, 1H), 3.54 (m, 1H), 3.44 (m, 2H), 3.21 (m, 1H), 2.57 (m, 2H), 1.77 (m, 1H), 1.42 (s, 9H), 0.93 (m, 1H). MS (ES) for C₂₇H₃₁N₅O₅5 found 566 (MH+).

### (±)-tert-Butyl (3S,4S)-1-(3-(2-aminopyrimidin-4-yl)-1H-pyrrolo[2,3-b]pyridin-4-yl)-3-hydroxypiperidin-4-ylcarbamate (66)

The title compound was synthesized in a manner similar to Example 63, with (±)-tert-butyl (3S,4S)-1-(3-(2-aminopyrimidin-4-yl)-1-(phenylsulfonyl)-1H-pyrrolo[2,3-b]pyrid hydroxypiperidin-4-ylcarbamate (65) instead of (S)-4-(1-(phenylsulfonyl)-4-(3-(3,3,3-trifluoropropylamino)pyrrolidin-1-yl)-1H-pyrrolo[2,3-b]pyridin-3-yl)pyrimidin-2-amine (46) as substrate.

¹H-NMR (400 MHz, CD₃OD): δ 8.25 (d, 1H), 8.10 (d, 1H), 7.80 (s, 1H), 7.09 (d, 1H), 6.80 (d, 1H), 3.62 (m, 1H), 3.51 (m, 1H), 3.41 (m, 1H), 3.33 (m, 1H), 2.71 (m, 1H), 2.56 (m, 1H), 1.81 (m, 1H), 1.51 (m, 1H), 1.44 (s, 9H). MS (ES) for C₂₁H₂₇N₁₇O₃, found 426 (MH+).

### (3S,4S)-4-Amino-1-[3-(2-aminopyrimidin-4-yl)-1H-pyrrolo[2,3-b]pyridin-4-yl]piperidin-3-ol

The title compound was synthesized in a manner similar to that above, with (±)-tert-butyl (3S,4S)-1-(3-(2-aminopyrimidin-4-yl)-1H-pyrrolo[2,3-b]pyridin-4-yl)-3-hydroxypiperidin-4-ylcarbamate (66) instead of (S)-tert-butyl 1-(3-(2-aminopyrimidin-4-yl)-1-(phenylsulfonyl)-1l-pyrrolo[2,3-b]pyridin-4-yl)pyrrolidin-3-yl(3,3,3-trifluoropropyl)carbamate (45) as substrate.

¹H-NMR (400 MHz, CD₃OD): δ 8.24 (m, 2H), 8.17 (d, 1H), 7.31 (d, 1H), 7.11 (d, 1H), 3.96 (m, 2H), 3.59 (m, 1H), 3.26 (m, 1H), 3.16 (m, 1H), 2.99 (m, 1H), 2.06 (m, 1H), 1.87 (m, 1H). MS (ES) for C₁₆H₁₉N₇O, found 326 (MH+).

### Example 69

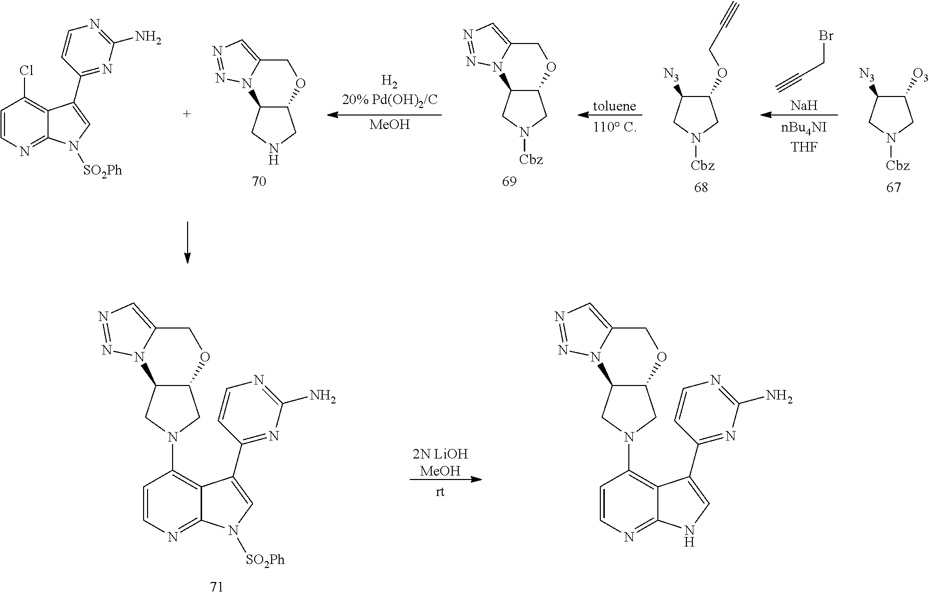

### (3R,4R)-Benzyl 3-azido-4-(prop-2-ynyloxy)pyrrolidine-1-carboxylate (68)

The title compound was made from compound (3R,4R)-benzyl 3-azido-4-(prop-2-ynyloxy)pyrrolidine-1-carboxylate (3R,4R)-benzyl 3-azido-4-hydroxypyrrolidine-1-carboxylate (67) following the known procedure as referenced in Pericàs, M. A. et al Org Lett 2008, 10, 1617. Compound 67 was prepared enantioselectively following the known procedure as referenced in Jacobsen, E. N. et al J Org Lett 1997, 62, 4197.

¹H-NMR (400 MHz, CDCl₃): δ 7.32 (m, 5H), 5.12 (s, 2H), 4.18 (m, 2H), 4.09 (m, 2H), 3.63 (m, 2H), 3.49 (m, 2H), 2.51 (m, 1H). ¹³C-NMR (100 MHz, CDCl₃): δ 154.9, 136.8, 128.8, 128.3, 128.2, 80.6, 79.8, 79.0, 76.0, 67.3, 63.6, 62.7, 57.2, 49.5, 49.1.

### (5aR,8aR)-Benzyl 5a,6,8,8a-tetrahydropyrrolo[3,4-b][1,2,3]triazolo[1,5-d][1,4]oxazine-7(4H)-carboxylate (69)

The title compound was made from compound (3R,4R)-benzyl 3-azido-4-(prop-2-ynyloxy)pyrrolidine-1-carboxylate (68) following the known procedure as referenced in Pericàs, M. A. et al Org Lett 2008, 10, 1617.

¹H-NMR (400 MHz, CDCl₃): δ 7.36 (m, 5H), 5.24 (m, 1H), 5.17 (s, 2H), 5.06 (m, 1H), 4.49 (m, 1H), 4.29 (m, 1H), 3.97 (m, 2H), 3.60 (m, 1H), 3.43 (m, 1H). ¹³C-NMR (100 MHz, CDCl₃): δ 152.9, 134.3, 128.7, 127.0, 126.7, 126.4, 126.2, 126.1, 65.5, 62.0, 56.1, 55.7, 44.2, 43.3.

### (5aR,8aR)-4,5a,6,7,8,8a-hexahydropyrrolo[3,4-b][1,2,3]triazolo[1,5-d][1,4]oxazine (70)

(5aR,8aR)-Benzyl 5a,6,8,8a-tetrahydropyrrolo[3,4-b][1,2,3]triazolo[1,5-d][1,4]oxazine-7(4H)-carboxylate (69, 437 mg, 1.455 mmol) was dissolved in methanol (15 mL), and the obtained solution transferred to a round bottom flask containing 20% Pd(OH)₂/C (37 mg). After purging with N₂, an ambient pressure H₂ atmosphere was established by insertion of a balloon. The mixture was kept stirring overnight, then filtered through paper and concentrated to dryness. The title compound was obtained as an oil (228 mg, 94% yield), which was deemed pure enough by TLC to undergo the next preparation without further purification and characterization.

### 4-(1-(Phenylsulfonyl)-4-((5aR,8aR)-5a,6,8,8a-tetrahydropyrrolo[3,4-b][1,2,3]triazolo[1,5-d][1,4]oxazin-7(4H)₄)-1H-pyrrolo[2,3-b]pyridin-3-yl)pyrimidin-2-amine (71)

The title compound was obtained by S_{N}Ar of 4-(4-chloro-1-(phenylsulfonyl)-1H-pyrrolo[2,3-b]pyridin-3-yl)pyrimidin-2-amine (314 mg, 0.813 mmol) with (5aR,8aR)-4,5a,6,7,8,8a-hexahydropyrrolo[3,4-b][1,2,3]triazolo[1,5-d][1,4]oxazine (70, 228 mg, 1.372 mmol) and DIEA in n-PrOH at 110° C. for 16 h, followed by purification by flash chromatography (91:8:1 dichloromethane/methanol/28% (w/w) ammonium hydroxide), as a solid (96 mg, 23% yield). The product was deemed pure enough to undergo the next preparation without further characterization. MS (ES) for C₂₄H₂₁N₉O₃S, found 516 (MH+).

### 4-{4-[(5aR,8aR)-5a,6,8,8a-tetrahydro-4H,7H-pyrrolo[3,4-b][1,2,3]triazolo[1,5-d][1,4]oxazin-7-yl]-1H-pyrrolo[2,3-b]pyridin-3-yl}pyrimidin-2-amine

The title compound was synthesized in a manner similar to that above, with 4-(1-(phenylsulfonyl)-44(5aR,8aR)-5a,6,8,8a-tetrahydropyrrolo[3,4-b][1,2,3]triazolo[1,5-d][1,4]oxazin-7(4H)-yl)-1H-pyrrolo[2,3-b]pyridin-3-yl)pyrimidin-2-amine (71) instead of (S)-4-(1-(phenylsulfonyl)-4-(3-(3,3,3-trifluoropropylamino)pyrrolidin-1-yl)-1H-pyrrolo[2,3-b]pyridin-3-yl)pyrimidin-2-amine (46) as substrate.

¹H-NMR (400 MHz, d₆-DMSO): δ 8.14 (d, 1H), 7.97 (d, 1H), 7.65 (s, 1H), 7.56 (d, 1H), 6.77 (d, 1H), 6.49 (m, 2H), 5.25 (d, 1H), 5.00 (d, 1H), 4.46 (m, 1H), 4.11 (m, 2H), 3.55 (m, 2H), 3.39 (m, 1H). MS (ES) for C₁₈H₁₇N₉O, found 376 (MH+).

### Example 70

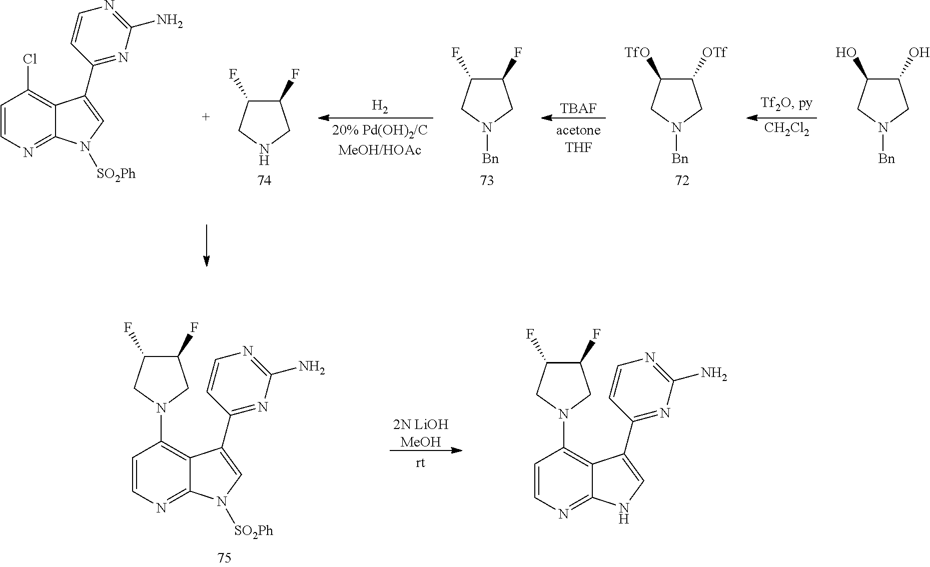

### 3R,4R)-1-benzylpyrrolidine-3,4-diyl bis(trifluoromethanesulfonate) (72)

To a solution of (3R,4R)-1-benzylpyrrolidine-3,4-diol (522 mg, 2.70 mmol) and pyridine (0.65 mL) in anhydrous CH₂Cl₂ (20 mL), Tf₂O (0.94 mL, 5.73 mmol) was added via syringe at −40° C. The reaction mixture was brought to rt 3.5 h later and left stir for an additional 30 min. The mixture was then filtered through MgSO₄, concentrated and purified by flash chromatography (83:17 hexanes/ethyl acetate), to give the title compound as an oil (1.069 g mg, 87% yield).

### (3S,4S)-1-benzyl-3,4-difluoropyrrolidine (73)

To a solution of (3R,4R)-1-benzylpyrrolidine-3,4-diyl bis(trifluoromethanesulfonate) (72, 1.069 g, 2.34 mmol) and acetone (25 mL), TBAF.3H₂O (1.80 g, 5.71 mmol, partially dried by two azeotropic distillations from toluene) was added as a solution in anhdrous THF (30 mL) via cannula. The mixture was stirred at rt for 24 h, then concentrated under reduced pressure and purified by flash chromatography (9:1 hexanes/ethyl acetate), to give the title compound as an oil (262 mg, 56% yield).

¹H-NMR (400 MHz, C₆D₆): δ 7.14 (m, 3H), 7.03 (m, 2H), 4.91 (m, 2H), 2.43 (m, 2H), 2.24 (m, 2H). ¹³C-NMR (100 MHz, C₆D₆): δ 133.2, 128.6, 127.9, 114.0-123.5 (q), 88.1, 58.0, 56.6. ¹⁹F-NMR (376 MHz, C₆D₆): 6-75.8 (s, 6F).

### (3S,4S)-3,4-difluoropyrrolidine (74)

The title compound was synthesized in a manner similar to Example 69, with (3S,4S)-1-benzyl-3,4-difluoropyrrolidine (73) instead of (5aR,8aR)-benzyl 5a,6,8,8a-tetrahydropyrrolo[3,4-b][1,2,3]triazolo[1,5-d][1,4]oxazine-7(4H)-carboxylate (69) as substrate. Upon completion of reaction, Bio-Rad® AG 1-x8 resin (100 mg, hydroxide form) was added to the reaction mixture, which was stirred at rt for 10 min, until a basic pH was reached, finally filtered through paper. The obtained clear solution was concentrated to a volume of 3 mL and used as such in the next preparation.

### 4-(4-((3S,4S)-3,4-Difluoropyrrolidin-14)-1-(phenylsulfonyl)-1H-pyrrolo[2,3-b]pyridin-3-yl)pyrimidin-2-amine (75)

The title compound was obtained by S_{N}Ar of 4-(4-chloro-1-(phenylsulfonyl)-1H-pyrrolo[2,3-b]pyridin-3-yl)pyrimidin-2-amine (127 mg, 0.329 mmol) with (3S,4S)-3,4-difluoropyrrolidine (74, solution in MeOH, 1.3 mmol) and DIEA in n-BuOH at 120° C. for 16 h, followed by purification by flash chromatography (91:8:1 dichloromethane/methanol/28% (w/w) ammonium hydroxide), as a solid (77 mg, 51% yield).

¹H-NMR (400 MHz, CDCl₃/CD₃OD): δ 8.24 (m, 1H), 8.22 (m, 3H), 8.00 (s, 1H), 7.58 (m, 1H), 7.51 (m, 2H), 6.89 (d, 1H), 6.56 (d, 1H), 5.34 (m, 1H), 5.21 (m, 1H), 3.58 (m, 1H), 3.44 (m, 1H), 3.38 (m, 2H). ¹⁹F-NMR (376 MHz, CDCl₃/CD₃OD): 6-188.5 (m, 2F). MS (ES) for C₂₁H₁₈F₂N₆O₂S, found 457 (MH+).

### 4-{4-[(3S,4S)-3,4-Difluoropyrrolidin-1-yl]-1H-pyrrolo[2,3-b]pyridin-3-yl}pyrimidin-2-amine

The title compound was synthesized in a manner similar to that above, with 4-(4-((3S,4S)-3,4-difluoropyrrolidin-1-yl)-1-(phenylsulfonyl)-1H-pyrrolo[2,3-b]pyridin-3-yl)pyrimidin-2-amine (75) instead of (S)-4-(1-(phenylsulfonyl)-4-(3-(3,3,3-trifluoropropylamino)pyrrolidin-1-yl)-1H-pyrrolo[2,3-b]pyridin-3-yl)pyrimidin-2-amine (46) as substrate.

¹H-NMR (400 MHz, CDCl₃/CD₃OD): δ 8.22 (d, 1H), 8.01 (d, 1H), 7.92 (s, 1H), 7.14 (d, 1H), 6.80 (d, 1H), 5.34 (m, 1H), 5.21 (m, 1H), 3.89-3.95 (m, 4H). ¹⁹F-NMR (376 MHz, CDCl₃/CD₃OD): 6-188.9 (m, 2F). MS (ES) for C₁₅H₁₄F₂N₆, found 317 (MH+).

### Example 71

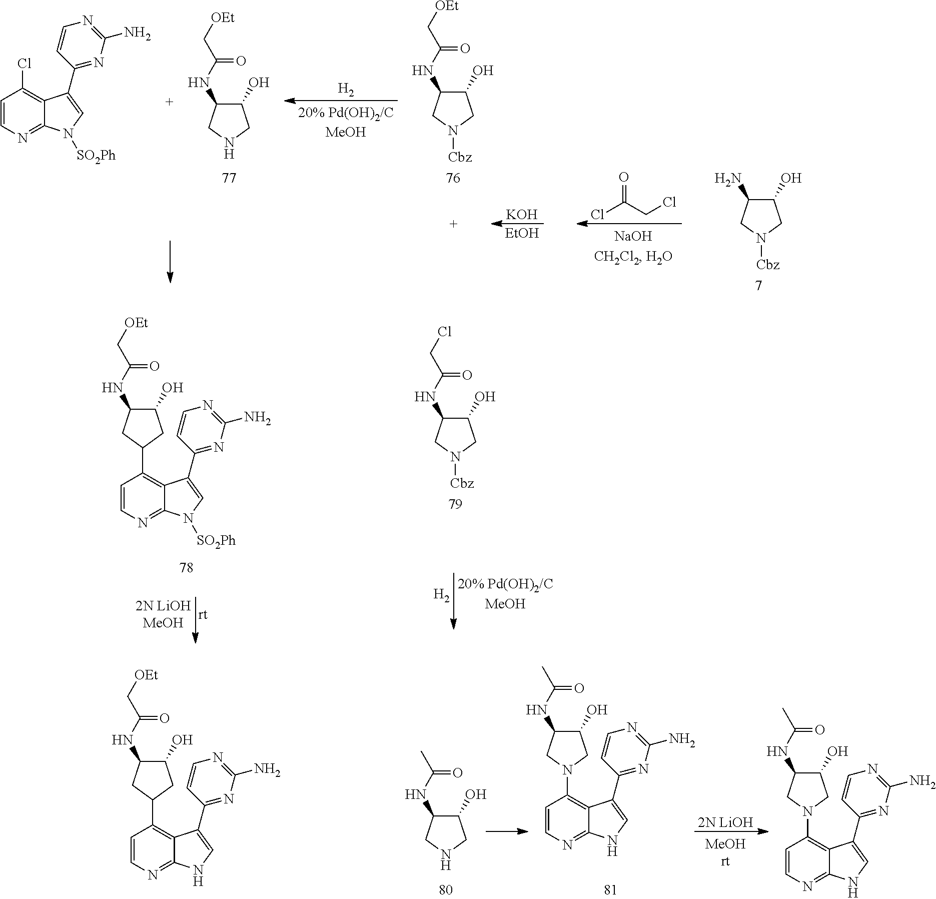

### (3R,4R)-Benzyl 3-(2-ethoxyacetamido)-4-hydroxypyrrolidine-1-carboxylate (76) and (3R,4R)-benzyl 3-(2-chloroacetamido)-4-hydroxypyrrolidine-1-carboxylate (79)

Compound 7 (see Example 3, 295 mg, 1.25 mmol) was treated with chloroacetyl chloride (0.14 mL, 1.76 mmol) and NaOH (flakes; 93 mg) in CH₂Cl₂ (10 mL) and water (0.07 mL) at rt for 16 h. Upon com2pletion of the reaction, the mixture was diluted with ethyl acetate and washed with aq citric acid. The aqueous phase was extracted with ethyl acetate. The combined organic layers were washed with 1M potassium carbonate, water, brine, finally dried over MgSO₄. After purification by flash chromatography (91:8:1 dichloromethane/methanol/28% (w/w) ammonium hydroxide), the title compounds 76 and 79 were obtained in an inseparable mixture, which underwent the following preparation without further treatment.

### (3R,4R)-benzyl 3-(2-ethoxyacetamido)-4-hydroxypyrrolidine-1-carboxylate (77) and N-((3R,4R)-4-hydroxypyrrolidin-3-yl)acetamide (80)

The title compounds were synthesized in a manner similar to that above, with the mixture of (3R,4R)-benzyl 3-(2-ethoxyacetamido)-4-hydroxypyrrolidine-1-carboxylate (76) and (3R,4R)-benzyl 3-(2-chloroacetamido)-4-hydroxypyrrolidine-1-carboxylate (79) instead of (5aR,8aR)-benzyl 5a,6,8,8a-tetrahydropyrrolo[3,4-b][1,2,3]triazolo[1,5-d][1,4]oxazine-7(4H)-carboxylate (69) as substrates. The title compounds were obtained as pure oils after purification by flash chromatography (88:10:2 dichloromethane/methanol/28% (w/w) ammonium hydroxide); compound 77-89 mg (38% yield over two steps); compound 80-41 mg (22% yield over two steps).

Compound 77: ¹H-NMR (400 MHz, CDCl₃): δ 6.94 (m, 1H), 4.12 (m, 2H), 4.01 (m, 1H), 3.92 (s, 2H), 3.56 (q, 2H), 3.48 (m, 1H), 3.08 (dd, 1H), 2.93 (m, 1H), 2.77 (dd, 1H), 1.23 (t, 3H). ¹³C-NMR (100 MHz, CDCl₃): δ 170.9, 69.9, 67.3, 58.8, 53.4, 51.2, 15.2.

Compound 80: ¹H-NMR (400 MHz, CDCl₃/CD₃OD): δ 4.13 (m, 1H), 4.06 (m, 1H), 3.40 (dd, 1H), 3.09 (m, 1H), 2.91 (m, 1H), 2.80 (m, 1H), 1.97 (s, 3H). ¹³C-NMR (100 MHz, CDCl₃/CD₃OD): δ 172.2, 76.2, 58.2, 52.4, 49.9, 22.2.

### N-((3R,4R)-1-(3-(2-Aminopyrimidin-4-yl)-1-(phenylsulfonyl)-1H-pyrrolo[2,3-b]pyridin-4-yl)-4-hydroxypyrrolidin-3-yl)-2-ethoxyacetamide (78)

The title compound was obtained by S_{N}Ar of 4-(4-chloro-1-(phenylsulfonyl)-1H-pyrrolo[2,3-b]pyridin-3-yl)pyrimidin-2-amine (101 mg, 0.26 mmol) with (3R,4R)-benzyl 3-(2-ethoxyacetamido)-4-hydroxypyrrolidine-1-carboxylate (77, 89 mg, 0.47 mmol) and DIEA in n-BuOH at 120° C. for 16 h, followed by purification by flash chromatography (91:8:1 dichloromethane/methanol/28% (w/w) ammonium hydroxide), as a solid (61 mg, 44% yield).

¹H-NMR (400 MHz, CDCl₃): δ 8.25 (m, 1H), 8.22 (m, 2H), 8.16 (m, 1H), 7.95 (s, 1H), 7.56 (m, 1H), 7.47 (m, 2H), 6.87 (d, 1H), 6.69 (m, 1H), 6.51 (m, 1H), 5.48 (m, 2H), 4.81 (m, 1H), 4.13 (m, 2H), 3.84 (dd, 2H), 3.62 (m, 1H), 3.53 (q, 2H), 3.41 (m, 1H), 2.00 (m, 1H), 1.17 (t, 3H). ¹³C-NMR (100 MHz, CDCl₃): δ 170.9, 163.1, 162.8, 150.9, 146.2, 138.0, 134.5, 129.2, 128.6, 125.3, 119.4, 110.3, 109.4, 105.5, 69.7, 67.4, 57.4, 56.5, 53.7, 15.1. MS (ES) for C₂₅H₂₇N₇O₅S, found 538 (MH+).

### N-{(3R,4R)-1-[3-(2-aminopyrimidin-4-yl)-1H-pyrrolo[2,3-b]pyridin-4-yl]-4-hydroxypyrrolidin-3-yl}-2-(ethyloxy)acetamide

The title compound was synthesized in a manner similar to that above, with N-((3R,4R)-1-(3-(2-aminopyrimidin-4-yl)-1-(phenylsulfonyl)-1H-pyrrolo[2,3-b]pyridin-4-yl)-4-hydroxypyrrolidin-3-yl)-2-ethoxyacetamide (78) instead of (S)-4-(1-(phenylsulfonyl)-4-(3-(3,3,3-trifluoropropylamino)pyrrolidin-1-yl)-1H-pyrrolo[2,3-b]pyridin-3-yl)pyrimidin-2-amine (46) as substrate.

¹H-NMR (400 MHz, CD₃OD): δ 8.18 (d, 1H), 7.98 (d, 1H), 7.62 (s, 1H), 6.94 (d, 1H), 6.57 (d, 1H), 4.15 (m, 2H), 3.92 (dd, 2H), 3.69 (m, 1H), 3.57 (q+m, 3H), 3.30 (m, 1H), 3.01 (m, 1H), 1.22 (t, 3H). ¹⁹F-NMR (376 MHz, CD₃OD): 6-188.9 (m, 2F). MS (ES) for C₁₉H₂₃N₇O₃, found 398 (MH+).

### N-{(3R,4R)-1-[3-(2-aminopyrimidin-4-yl)-1H-pyrrolo[2,3-b]pyridin-4-yl]-4-hydroxypyrrolidin-3-yl}acetamide

N-((3R,4R)-1-(3-(2-Aminopyrimidin-4-yl)-1H-pyrrolo[2,3-b]pyridin-4-yl)-4-hydroxypyrrolidin-3-yl)acetamide (81) was obtained by S_{N}Ar of 4-(4-chloro-1-(phenylsulfonyl)-1H-pyrrolo[2,3-b]pyridin-3-yl)pyrimidin-2-amine (69 mg, 0.179 mmol) with N-((3R,4R)-4-hydroxypyrrolidin-3-yl)acetamide (80, 41 mg, 0.288 mmol) and DIEA in n-BuOH at 120° C. for 16 h. After cooling to rt, the mixture was treated in a manner similar to Example 21, wherein intermediate compound 81 was used instead of (S)-4-(1-(phenylsulfonyl)-4-(3-(3,3,3-trifluoropropylamino)pyrrolidin-1-yl)-1H-pyrrolo[2,3-b]pyridin-3-yl)pyrimidin-2-amine (46) as substrate, to give the title compound as a solid.

¹H-NMR (400 MHz, CD₃OD): δ 8.17 (d, 1H), 7.95 (d, 1H), 7.57 (s, 1H), 6.92 (d, 1H), 4.06 (m, 2H), 3.68 (m, 1H), 3.52 (m, 1H), 3.30 (m, 1H), 3.15 (m, 1H), 3.06 (m, 1H), 1.94 (s, 3H). ¹³C-NMR (100 MHz, CD₃OD): δ 172.2, 164.5, 163.2, 157.5, 151.9, 150.5, 143.8, 125.9, 115.6, 109.5, 107.1, 101.9, 73.4, 56.9, 56.3, 53.2, 21.4. MS (ES) for C₁₇H₁₉N₇O₂, found 354 (MH+).

### Example 72

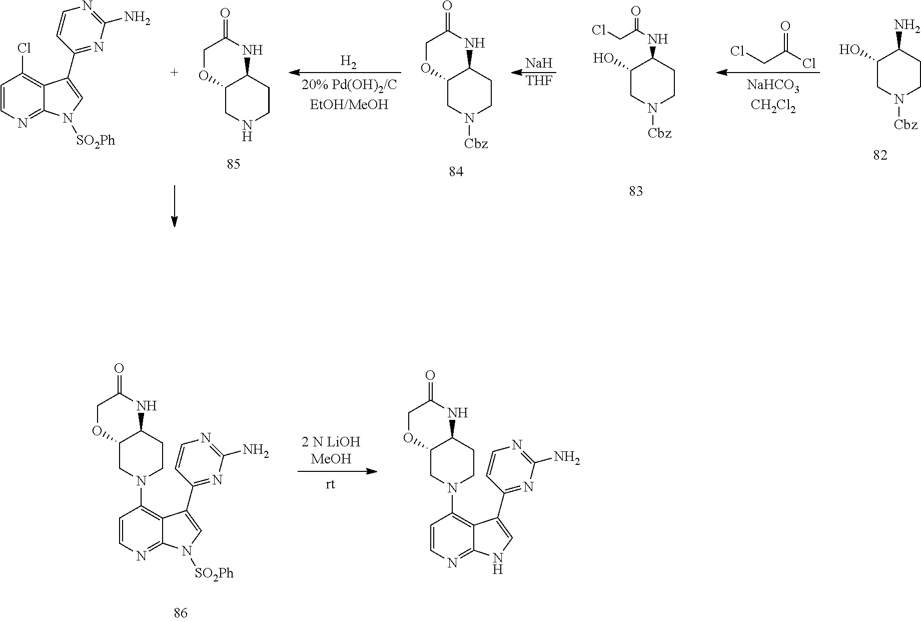

### (±)-(4aS,8aS)-Benzyl 2-oxohexahydro-1H-pyrido[3,4-b][1,4]oxazine-6(7H)-carboxylate (84)

Compound 82, which was prepared following the known procedure as referenced in WO2005/066176, was treated in a manner similar to Example 71, wherein compound 7 was used as substrate, to give intermediate (±)-(3S,4S)-benzyl 4-(2-chloroacetamido)-3-hydroxypiperidine-1-carboxylate (83), 711 mg (80% yield), which underwent the following step without purification nor characterization. To a suspension of NaH (60% oil dispersion; 265 mg, 6.63 mmol) in anhydrous THF (15 mL), a solution of compound 83 (2.18 mmol) was added via cannula at 0° C. The reaction mixture was allowed to warm to rt, then gradually to 50° C., at which temperature it was stirred for 24 h. The mixture was poured over a saturated solution of ammonium chloride, and extracted twice with ethyl acetate. The combined organic layers were washed with water, brine, finally dried over MgSO₄. After purification by flash chromatography (97:3 dichloromethane/methanol), the title compound was obtained as an oil (331 mg, 52% yield).

¹H-NMR (400 MHz, CDCl₃): δ 8.07 (m, 1H), 7.33 (m, 5H), 5.13 (s, 2H), 4.26 (m, 2H), 4.19 (dd, 2H), 3.30 (m, 2H), 2.75 (m, 2H), 1.89 (m, 1H), 1.47 (m, 1H). ¹³C-NMR (100 MHz, CDCl₃): δ 169.8, 155.3, 136.5, 128.8, 128.5, 128.2, 74.3, 67.9, 54.9, 46.2, 42.2, 29.7.

### (±)-(4aS,8aS)-hexahydro-1H-pyrido[3,4-b][1,4]oxazin-2(3H)-one (85)

The title compounds were synthesized in a manner similar to Example 69, with (±)-(4aS,8aS)-benzyl 2-oxohexahydro-1H-pyrido[3,4-b][1,4]oxazine-6(7H)-carboxylate (84) instead of (5aR,8aR)-benzyl 5a,6,8,8a-tetrahydropyrrolo[3,4-b][1,2,3]triazolo[1,5-d][1,4]oxazine-7(4H)-carboxylate (69) as substrate.

¹H-NMR (400 MHz, CDCl₃): δ 7.64 (m, 1H), 4.27 (dd, 2H), 3.28 (m, 3H), 3.04 (m, 1H), 2.60 (m, 2H), 1.87 (m, 2H), 1.48 (m, 1H).

### (±)-(4aS,8aS)-6-(3-(2-Aminopyrimidin-4-yl)-1-(phenylsulfonyl)-1H-pyrrolo[2,3-b]pyridin-4-yl)hexahydro-1H-pyrido[3,4-b][1,4]oxazin-2(3H)-one (86)

The title compound was obtained by S_{N}Ar of 4-(4-chloro-1-(phenylsulfonyl)-1H-pyrrolo[2,3-b]pyridin-3-yl)pyrimidin-2-amine (288 mg, 0.746 mmol) with (±)-(4aS,8aS)-hexahydro-1H-pyrido[3,4-b][1,4]oxazin-2(3H)-one (85, 180 mg, 1.14 mmol) and DIEA in n-BuOH at 130° C. for 16 h, followed by purification by flash chromatography (90:9:1 dichloromethane/methanol/28% (w/w) ammonium hydroxide), as a solid (230 mg, 60% yield), which was utilized in the following preparation without further characterization.

### (4aR,8aR)-6-[3-(2-Aminopyrimidin-4-yl)-1H-pyrrolo[2,3-b]pyridin-4-yl]hexahydro-1H-pyrido[3,4-b][1,4]oxazin-2(3H)-one

The title compound was synthesized in a manner similar to that above, with (±)-(4aS,8aS)-6-(3-(2-aminopyrimidin-4-yl)-1-(phenylsulfonyl)-1H-pyrrolo[2,3-b]pyridin-4-yl)hexahydro-1H-pyrido[3,4-b][1,4]oxazin-2(3H)-one instead of (S)-4-(1-(phenylsulfonyl)-4-(3-(3,3,3-trifluoropropylamino)pyrrolidin-1-yl)-1H-pyrrolo[2,3-b]pyridin-3-yl)pyrimidin-2-amine (46) as substrate.

¹H-NMR (400 MHz, d₆-dmso): δ 12.05 (m, 1H), 8.28 (m, 1H), 8.24 (d, 1H), 8.09 (m, 1H), 7.72 (m, 1H), 6.89 (d, 1H), 6.70 (d, 1H), 6.49 (m, 1H), 4.08 (d, 1H), 3.97 (d, 1H), 3.47 (m, 2H), 3.36 (m, 1H), 3.17 (m, 1H), 2.60 (m, 2H), 1.71 (m, 1H), 1.45 (m, 1H). MS (ES) for C₁₈H₁₉N₇O₃, found 366 (MH+).

### Example 73

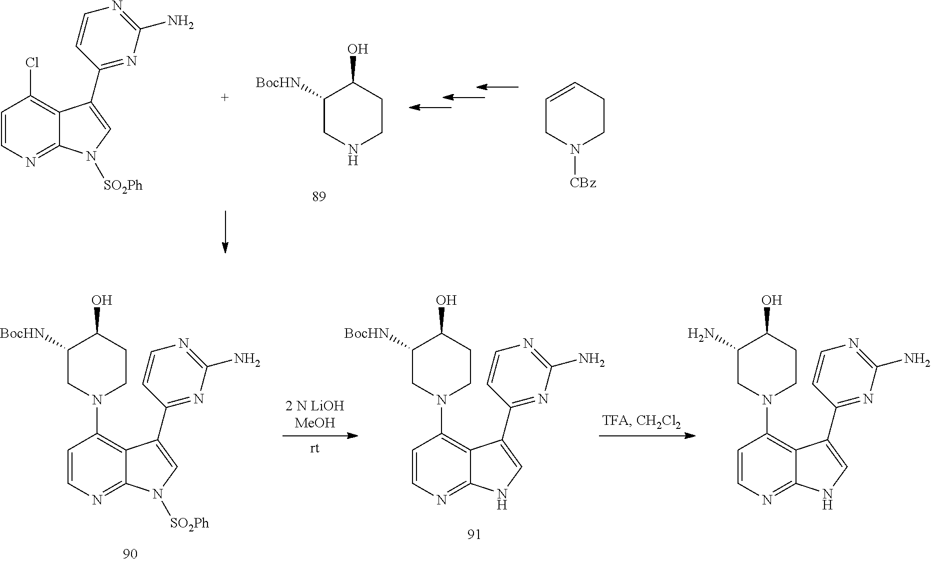

### (±)-tert-Butyl (3S,4S)-1-(3-(2-aminopyrimidin-4-yl)-1-(phenylsulfonyl)-1H-pyrrolo[2,3-b]pyridin-4-yl)-4-hydroxypiperidin-3-ylcarbamate (90)

The title compound was obtained by S_{N}Ar of 4-(4-chloro-1-(phenylsulfonyl)-1H-pyrrolo[2,3-b]pyridin-3-yl)pyrimidin-2-amine (73 mg, 0.189 mmol) with (±)-tert-butyl (3S,4S)-4-hydroxypiperidin-3-ylcarbamate (89, 80 mg, 0.37 mmol) and DIEA in n-PrOH at 110° C. for 16 h, followed by purification by flash chromatography (90:9:1 dichloromethane/methanol/28% (w/w) ammonium hydroxide), as a solid (106 mg, 99% yield).

¹H-NMR (400 MHz, CDCl₃): δ 8.28 (d, 1H), 8.25 (m, 1H), 8.21 (m, 2H), 7.96 (s, 1H), 7.58 (m, 1H), 7.48 (m, 2H), 6.92 (m, 1H), 6.73 (d, 1H), 3.65 (m, 2H), 3.49 (m, 1H), 2.99 (m, 1H), 2.82 (m, 1H), 2.76 (m, 1H), 1.95 (m, 1H), 1.51 (m, 1H), 1.46 (s, 9H). MS (ES) for C₂₁H₂₇N₇O₃, found 426 (MH+).

### (±)-tert-Butyl (3S,4S)-1-(3-(2-aminopyrimidin-4-yl)-1H-pyrrolo[2,3-b]pyridin-4-yl)-4-hydroxypiperidin-3-ylcarbamate (91)

The title compound was synthesized in a manner similar to that above Example 63, with (±)-tert-butyl (3S,4S)-1-(3-(2-aminopyrimidin-4-yl)-1-(phenylsulfonyl)-1H-pyrrolo[2,3-b]pyridin-4-yl)-4-hydroxypiperidin-3-ylcarbamate (90) instead of (S)-4-(1-(phenylsulfonyl)-4-(3-(3,3,3-trifluoropropylamino)pyrrolidin-1-yl)-1H-pyrrolo[2,3-b]pyridin-3-yl)pyrimidin-2-amine (46) as substrate.

¹H-NMR (400 MHz, CDCl₃/CD₃OD): δ 8.25 (d, 1H), 8.11 (d, 1H), 7.69 (s, 1H), 7.04 (m, 1H), 6.79 (d, 1H), 3.70 (m, 2H), 3.57 (m, 1H), 3.35 (m, 1H), 3.22 (m, 1H), 2.83 (m, 2H), 1.73 (m, 1H), 1.46 (s, 9H). MS (ES) for C₂₁H₂₇N₇O₃, found 426 (MH+).

### (3R,4R)-3-Amino-1-[3-(2-aminopyrimidin-4-yl)-1H-pyrrolo[2,3-b]pyridin-4-yl]piperidin-4-ol

The title compound was synthesized in a manner similar to Example 63, with (±)-tert-butyl (3S,4S)-1-(3-(2-aminopyrimidin-4-yl)-1H-pyrrolo[2,3-b]pyridin-4-yl)-4-hydroxypiperidin-3-ylcarbamate (91) instead of instead of (S)-tert-butyl 1-(3-(2-aminopyrimidin-4-yl)-1-(phenylsulfonyl)-1l-pyrrolo[2,3-b]pyridin-4-yl)pyrrolidin-3-yl(3,3,3-trifluoropropyl)carbamate (45) as substrate.

¹H-NMR (400 MHz, CDCl₃/CD₃OD): δ 8.24 (d, 1H), 8.12 (d, 1H), 7.77 (s, 1H), 7.09 (d, 1H), 6.77 (d, 1H), 3.61 (m, 1H), 3.45 (m, 1H), 3.34 (m, 1H), 2.86 (m, 1H), 2.65 (m, 1H), 2.61 (m, 1H), 1.80 (m, 1H), 1.57 (m, 1H). MS (ES) for C₁₆H₁₉N₇O, found 326 (MH+).

### Example 74

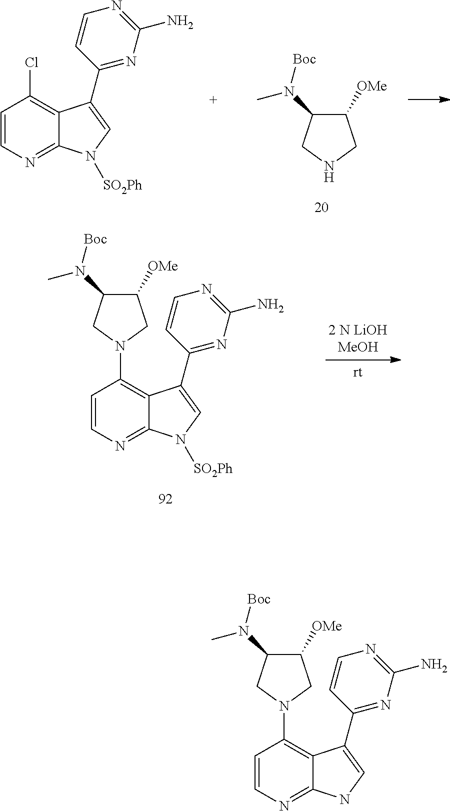

### 1,1-Dimethylethyl[(3R,4R)-1-[3-(2-aminopyrimidin-4-yl)-1H-pyrrolo[2,3-b]pyridin-4-yl]-4-(methyloxy)Pyrrolidin-3-yl]methylcarbamate

The title compound was synthesized in a manner similar to Example 48.

¹H-NMR (400 MHz, CDCl₃): δ 8.20 (d, 1H), 8.04 (d, 1H), 7.59 (s, 1H), 6.94 (d, 1H), 6.52 (d, 1H), 4.40 (m, 3H), 3.87 (m, 1H), 3.68 (dd, 1H), 3.56 (m, 1H), 3.31 (s, 3H), 3.21 (m, 2H), 2.81 (s, 3H), 1.47 (s, 9H). MS (ES) for C₂₂H₂₉N₇O₃, found 440 (MH+).

### Example 75

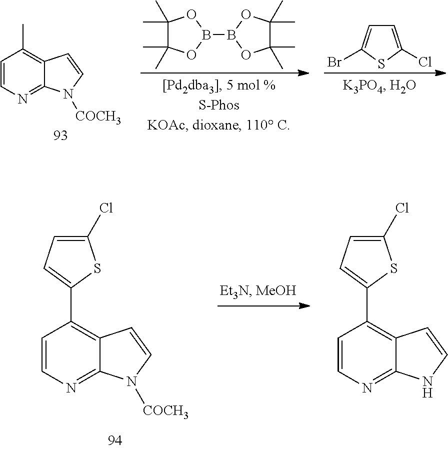

### 1-(4-Iodo-1H-pyrrolo[2,3-b]pyridin-1-yl)ethanone (93)

Compound 93 was made following the known procedure as referenced in Corcoran, R. C. et al Tetrahedron Lett 1990, 31, 6757.

### 1-(4-(5-Chlorothiophen-2-yl)-1H-pyrrolo[2,3-b]pyridin-1-yl)ethanone (94)

A pressure tube was charged with 1-(4-iodo-1H-pyrrolo[2,3-b]pyridin-1-yl)ethanone (93, 110 mg, 0.38 mmol), bis(pinacolato)diboro (332 mg, 1.31 mmol), tris(dibenzylideneacetone)palladium(0) (9.1 mg, 0.01 mmol), Sphos (38 mg, 0.09 mmol), potassium acetate (214 mg, 2.18 mmol), and ahydrous 1,4-dioxane (4 mL). After purging the mixture with nitrogen gas, the tube was sealed and heated at 110° C. for 18 h. The reaction mixture was allowed to cool to room temperature. 2-Bromo-5-chlorothiophene (131 mg, 0.66 mmol), potassium phosphate (271 mg, 1.28 mmol), water (0.25 mL) and a second aliquot of tris(dibenzylideneacetone)palladium(0) (6.3 mg, 0.07 mmol), Sphos (26 mg, 0.06 mmol) were added. After purging the mixture with nitrogen gas, the tube was sealed and heated at 110° C. for 4 h. The mixture was cooled to room temperature, poured onto water and extracted twice with ethyl acetate. The combined organic layers were wshed with brine and dried over MgSO₄. The title compound was obtained after purification by flash chromatography (85:15 hexanes/ethyl acetate) as a solid (47 mg, 40% yield) of estimated 85% purity.

¹H-NMR (400 MHz, CDCl₃): δ 8.36 (d, 1H), 8.07 (d, 1H), 7.34 (d, 1H), 7.28 (d, 1H), 7.02 (d, 1H), 6.94 (m, 1H), 3.07 (s, 3H). MS (ES) for C₁₃H₉ClN₂OS, found 277 (MH+).

### 4-(5-Chloro-2-thienyl)-1H-pyrrolo[2,3-b]pyridine

A solution of 1-(4-(5-chlorothiophen-2-yl)-1H-pyrrolo[2,3-b]pyridin-1-yl)ethanone (94, 47 mg, 0.17 mmol) and triethylamine (1 mL) in methanol (5 mL) was stirred at 60° C. for 2 h. After removal of volatiles under reduced pressure, the crude reaction material was purified twice by flash chromatography (70:30:5 hexanes/ethyl acetate/methanol), to give the title compound as a solid (2 mg, 5% yield).

¹H-NMR (400 MHz, CDCl₃): δ 10.35 (m, 1H), 8.33 (d, 1H), 7.44 (m, 1H), 7.42 (d, 1H), 7.22 (d, 1H), 7.02 (d, 1H), 6.85 (m, 1H). MS (ES) for C₁₁H₇ClN₂S, found 235 (MH+).

### Example 76

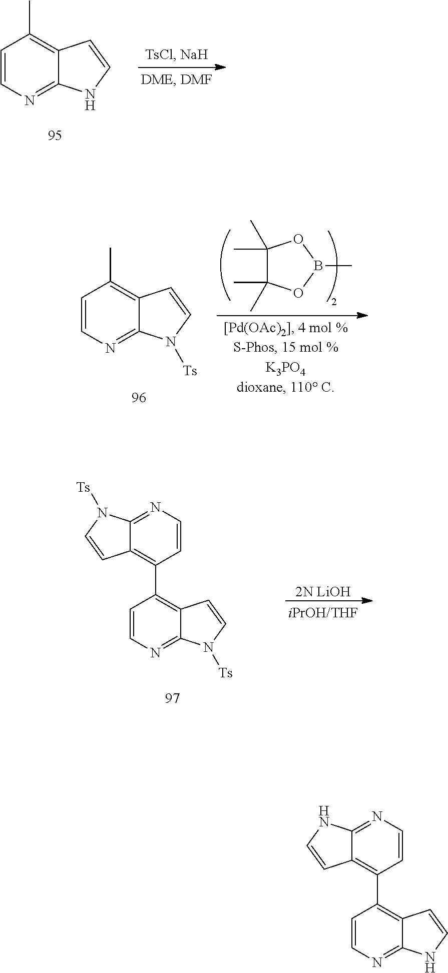

### 4-Iodo-1H-pyrrolo[2,3-b]pyridine (95)

Compound 95 was made following the known procedure as referenced in Corcoran, R. C. et al Tetrahedron Lett 1990, 31, 6757.

### 4-iodo-1-tosyl-1H-pyrrolo[2,3-b]pyridine (96)

To a suspension of sodium hydride (60% oil dispersion; 1.206 g, 30 mmol) in anhydrous DME, a solution of 4-iodo-1H-pyrrolo[2,3-b]pyridine (95, 4.73 g, 19.4 mmol) in DMF was added via cannula at 0° C. After stirring the mixture for 30 min at 0° C., a solution of tosyl chloride (4.149 g, 21.8 mmol) in DME was added via cannula. The mixture was allowed to gradually warm to rt, and stirred for another 16 h. The mixture was poured over a saturated solution of ammonium chloride, then extracted with ethyl acetate. The organic layer was washed with water (4×), brine, finally dried over MgSO₄. After purification by flash chromatography (7:3 hexanes/ethyl acetate), the title compound was obtained as a solid (5.674 g, 74% yield).

¹H-NMR (400 MHz, CDCl₃): δ 8.05 (m, 3H), 7.79 (m, 1H), 7.57 (d, 1H), 7.27 (d, 2H), 6.50 (d, 1H), 2.37 (s, 3H). MS (ES) for C₁₄H₁₁₁N₂O₂S, found 399 (MH+).

### 1,1′-Ditosyl-1H,1′H-4,4′-bipyrrolo[2,3-b]pyridine (97)

According to a procedure described in Buchwald, S. L. et al Angew Chem Int Ed 2007, 46, 5359, a pressure tube was charged with 4-iodo-1-tosyl-1H-pyrrolo[2,3-b]pyridine (96, 128 mg, 0.32 mmol), bis(pinacolato)diboro (99 mg, 0.39 mmol), palladium diacetate trimer (3.0 mg, 0.013 mmol), Sphos (11.5 mg, 0.028 mmol), potassium phosphate (208 mg, 0.98 mmol), and anhydrous 1,4-dioxane (1.5 mL). After purging the mixture with nitrogen gas, the tube was sealed and heated at 80° C. for 18 h. The mixture was cooled to room temperature, poured onto water and extracted twice with ethyl acetate. The combined organic layers were washed with brine and dried over MgSO₄. The title compound was obtained after purification by flash chromatography (90:9:1 dichloromethane/methanol/28% (w/w) ammonium hydroxide) as a solid (27 mg, 31% yield).

¹H-NMR (400 MHz, CDCl₃): δ 8.53 (m, 2H), 8.13 (m, 2H), 7.78 (m, 2H), 7.29 (m, 8H), 2.39 (s, 6H). ¹³C-NMR (100 MHz, CDCl₃): δ 148.01, 145.7, 145.3, 139.1, 135.5, 130.0, 128.5, 127.3, 121.2, 118.8, 104.5, 25.2, 21.9. MS (ES) for C₂₈H₂₂N₄O₄S₂, found 543 (MH+).

### 1H, l′H-4,4′-bipyrrolo[2,3-b]pyridine

The title compound was synthesized in a manner similar to Example 63, with 1,1′-ditosyl-1 H,1′H-4,4′-bipyrrolo[2,3-b]pyridine (97) instead of (S)-4-(1-(phenylsulfonyl)-4-(3-(3,3,3-trifluoropropylamino)pyrrolidin-1-O-1H-pyrrolo[2,3-b]pyridin-3-yl)pyrimidin-2-amine (46) as substrate, and by heating the reaction mixture at 60° C. for 6 h, instead of rt.

¹H-NMR (400 MHz, d₆-dmso): δ 11.9 (br s, 2H), 8.36 (d, 2H), 7.57 (m, 2H), 7.33 (d, 2H), 6.45 (m, 2H). MS (ES) for C₁₄H₁₀N₄, found 235 (MH+).

### Example 77

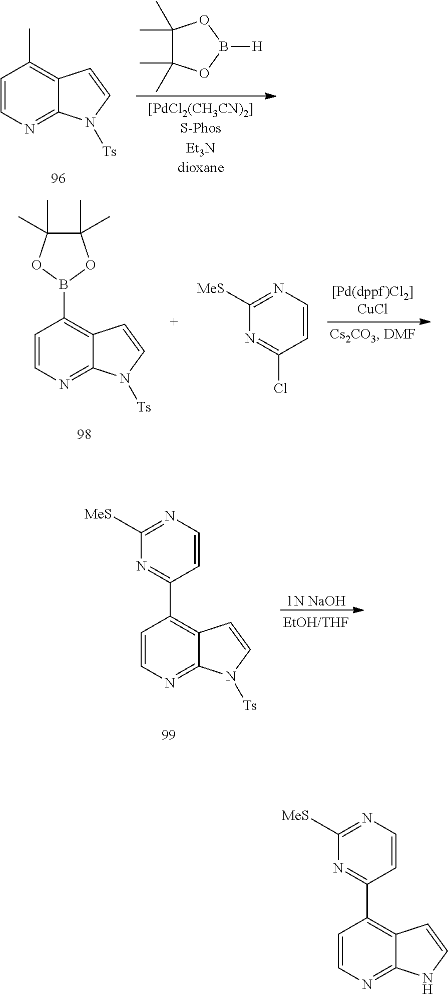

### 5,5-tetramethyl-1,3,2-dioxaborolan-2-yl)-1-tosyl-1H-pyrrolo[2,3-b]pyridine (98)

According to a procedure described in Buchwald, S. L. et al J Org Chem 2008, 73, 5589, a pressure tube was charged with 4-iodo-1-tosyl-1H-pyrrolo[2,3-b]pyridine (96, 314 mg, 0.789 mmol), pinacolborane (0.28 mL, 2.3 mmol), bis(acetonitrile)dichloropalladium(II) (5.9 mg, 0.023 mmol), Sphos (36.0 mg, 0.088 mmol), triethylamine (0.33 mL, 2.4 mmol), and ahydrous 1,4-dioxane (2 mL). After purging the mixture with nitrogen gas, the tube was sealed and heated at 100° C. for 18 h. The mixture was cooled to room temperature, concentrated to dryness, taken up with toluene/dichloromethane and directly loaded onto a silica gel column.

The title compound was obtained after purification by flash chromatography (8:2 hexanes/ethyl acetate) as a solid (264 mg, 84% yield).

¹H-NMR (400 MHz, CDCl₃): δ 8.30 (d, 1H), 8.10 (m, 2H), 7.78 (d, 1H), 7.49 (d, 1H), 7.05 (d, 1H), 6.57 (d, 2H), 1.66 (s, 3H), 1.01 (s, 12H). ¹³C-NMR (100 MHz, CDCl₃): δ 147.4, 144.5, 144.0, 136.3, 129.4, 128.4, 128.1, 127.7, 127.0, 124.9, 107.2, 84.1, 74.5, 67.0, 24.8, 24.7, 21.0.

### 4-(2-(Methylthio)pyrimidin-4-yl)-1-tosyl-1H-pyrrolo[2,3-b]pyridine (99)

According to a procedure described in Deng, Z. L. et al Org Lett 2009, 11, 345, a pressure tube was charged with 4-(4,4,5,5-tetramethyl-1,3,2-dioxaborolan-2-yl)-1-tosyl-1H-pyrrolo[2,3-b]pyridine (98, 144 mg, 0.36 mmol), 4-chloro-2-(methylthio)pyrimidine (30 μL, 0.26 mmol), 1,1′-bis(diphenylphosphino)ferrocene-palladium(II) dichloride dichloromethane adduct (10 mg, 0.012 mmol), cuprous chloride (23 mg, 0.23 mmol), cesium carbonate (158 mg, 0.48 mmol), and anhydrous DMF (2 mL). After purging the mixture with nitrogen gas, the tube was sealed and heated at 100° C. for 18 h. The mixture was cooled to room temperature, poured onto diluted ammonium hydroxide and extracted twice with ethyl acetate. The combined organic layers were washed with brine and dried over MgSO₄. The title compound was obtained after purification by flash chromatography (90:9:1 dichloromethane/methanol/28% (w/w) ammonium hydroxide) as a solid (18 mg, 17% yield).

¹H-NMR (400 MHz, CDCl₃): δ 8.65 (d, 1H), 8.55 (d, 1H), 8.08 (m, 2H), 7.86 (m, 1H), 7.60 (d, 1H), 7.40 (d, 1H), 7.27 (m, 2H), 7.22 (d, 1H), 2.64 (s, 3H), 2.37 (s, 3H). MS (ES) for C₁₉H₁₆N₄O₂S₂, found 397 (MH+).

### 4-[2-(Methylthio)pyrimidin-4-yl]-1H-pyrrolo[2,3-b]pyridine

The title compound was synthesized in a manner similar to Example 63, with 4-(2-(methylthio)pyrimidin-4-yl)-1-tosyl-1H-pyrrolo[2,3-b]pyridine (99) instead of (S)-4-(1-(phenylsulfonyl)-4-(3-(3,3,3-trifluoropropylamino)pyrrolidin-1-yl)-1H-pyrrolo[2,3-b]pyridin-3-yl)pyrimidin-2-amine (46) as substrate, NaOH instead of LiOH, and by heating the reaction mixture at 60° C. for 6 h, instead of rt.

¹H-NMR (400 MHz, CDCl₃/CD₃OD): δ 8.65 (m, 1H), 8.36 (m, 1H), 7.63 (dd, 2H), 7.59 (m, 1H), 7.04 (dd, 1H), 2.70 (s, 3H). MS (ES) for C₁₂H₁₀N₄S, found 243 (MH+).

### 4-(1H-Pyrrolo[2,3-b]pyridin-4-yl)pyridin-2-ol

The title compound was synthesized in a manner similar to Example 77, with 4-bromopyridin-2-ol instead of 4-chloro-2-(methylthio)pyrimidine and potassium phosphate instead of 2M sodium carbonate.

¹H-NMR (400 MHz, CDCl₃/CD₃OD): δ 8.30 (d, 1H), 7.52 (d, 1H), 7.48 (d, 1H), 7.20 (d, 1H), 6.93 (m, 1H), 6.76 (m, 1H), 6.67 (d, 1H). MS (ES) for C₁₂H₉N₃O, found 212 (MH+).

### Example 78

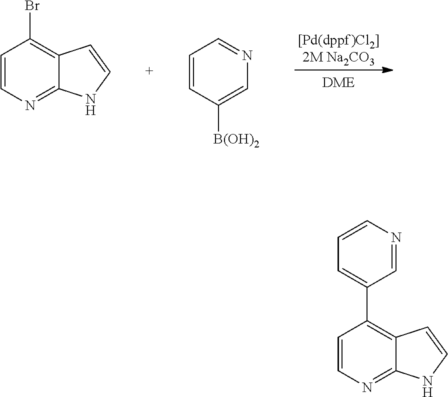

### 4-Pyridin-3-yl-1H-pyrrolo[2,3-b]pyridine

A thick-wall tube was charged with 4-bromo-1H-pyrrolo[2,3-b]pyridine (70 mg, 0.36 mmol), pyridin-3-ylboronic acid (72 mg, 0.59 mmol), 1,1′-bis(diphenylphosphino)ferrocene-palladium(II) dichloride dichloromethane adduct (14 mg, 0.017 mmol), a 2M solution of sodium carbonate (0.30 mL, 0.60 mmol), and ahydrous DME (2 mL). After purging the mixture with nitrogen gas, the tube was sealed and heated at 120° C. for 30 min in a CEM-Discover microwave reactor. The mixture was cooled to room temperature, poured onto water and extracted with ethyl acetate (70 mL) and dichloromethane (20 mL). The combined organic layers were washed with water, then with brine and dried over MgSO₄. The title compound was obtained after purification by flash chromatography (95:5 dichloromethane/methanol to 90:9:1 dichloromethane/methanol/28% (w/w) ammonium hydroxide) as a solid (53 mg, 77% yield).

¹H-NMR (400 MHz, CDCl₃/CD₃OD): δ 8.91 (d, 1H), 8.63 (m, 1H), 8.32 (d, 1H), 8.18 (m, 1H), 7.59 (m, 1H), 7.48 (d, 1H), 7.21 (d, 1H), 6.65 (d, 1H). ¹³C-NMR (100 MHz, CDCl₃/CD₃OD): δ 148.8, 148.6, 142.6, 138.4, 136.9, 135.4, 126.9, 124.4, 118.9, 114.8, 109.8, 99.3. MS (ES) for C₁₂H₉N₃, found 196 (MH+).

### 4-(1H-Indol-5-yl)-1H-pyrrolo[2,3-b]pyridine

The title compound was synthesized in a manner similar to Example 78, with 1-(tert-butoxycarbonyl)-1 H-indol-5-ylboronic acid instead of pyridin-3-ylboronic acid as reactant, followed by removal of the Boc group, as detailed in Example 21 with (S)-tert-butyl 1-(3-(2-aminopyrimidin-4-yl)-1-(phenylsulfonyl)-1l-pyrrolo[2,3-b]pyridin-4-yl)pyrrolidin-3-yl(3,3,3-trifluoropropyl)carbamate (45) as substrate.

¹H-NMR (400 MHz, CDCl₃/CD₃OD): δ 8.23 (d, 1H), 8.05 (s, 1H), 7.59 (d, 2H), 7.41 (d, 1H), 7.32 (m, 2H), 6.83 (d, 1H), 6.61 (d, 1H). MS (ES) for C₁₆H₁N₃, found 234 (MH+).

### 4-(5-Fluoropyridin-34)-1H-pyrrolo[2,3-b]pyridine

The title compound was synthesized in a manner similar to Example 78, with 5-fluoropyridin-3-ylboronic acid instead of pyridin-3-ylboronic acid as reactant.

¹H-NMR (400 MHz, CDCl₃/CD₃OD): δ 8.80 (d, 1H), 8.53 (d, 1H), 8.34 (d, 1H), 7.89 (m, 1H), 7.50 (m, 1H), 7.21 (d, 1H), 6.65 (d, 1H). MS (ES) for C₁₂H₈FN₃, found 214 (MH+).

### 4-(1H-Indol-4-yl)-1H-pyrrolo[2,3-b]pyridine

The title compound was synthesized in a manner similar to Example 78, with 4-(4,4,5,5-tetramethyl-1,3,2-dioxaborolan-2-yl)-1H-indole instead of pyridin-3-ylboronic acid as reactant.

¹H-NMR (400 MHz, CDCl₃/CD₃OD): δ 8.28 (d, 1H), 7.52 (d, 1H), 7.37 (m, 2H), 7.33 (m, 1H), 7.29 (m, 2H), 6.57 (m, 2H). MS (ES) for C₁₆H₁N₃, found 234 (MH+).

The title compound was synthesized in a manner similar to Example 78.

### 3-(2-aminopyrimidin-4-yl)-N-(1,2,2-trimethylpropyl)-1H-pyrrolo[2,3-b]pyridin-4-amine

¹H-NMR (400 MHz, CD₃OD): δ 8.10 (d, 1H), 7.82 (s, 1H), 7.77 (d, 1H), 7.08 (d, 1H), 6.37 (d, 1H), 3.67 (q, 1H), 1.32 (d, 3H), 1.97 (s, 9H). MS (ES) for C₁₇H₂₂N₆, found 311.2 (MH+).

Using analogous synthetic techniques as in Example 54, and substituting with the appropriate alternative starting materials, the following compounds were prepared. Alternative starting materials were obtained commercially unless otherwise indicated.

### (2S)-1-[3-(2-aminopyrimidin-4-yl)-1H-pyrrolo[2,3-b]pyridin-4-yl]piperidine-2-carboxamide

¹H-NMR (400 MHz, CD₃OD): δ 8.27 (d, 1H), 7.70 (s, 1H), 7.12 (d, 1H), 6.88 (d, 1H), 3.87 (dd, 1H), 3.12 (m, 1H), 2.52 (m, 1H), 2.07 (m, 1H), 1.82 (m, 2H), 1.40 (m, 2H), 1.14 (m, 1H). MS (ES) for C₁₇H₁₉N₇O, found 338.2 (MH+).

### (S)-{1-[3-(2-aminopyrimidin-4-yl)-1H-pyrrolo[2,3-b]pyridin-4-yl]piperidin-4-yl}(4-chlorophenyl)methanol

¹H-NMR (400 MHz, CD₃OD): δ 8.11 (d, 1H), 8.05 (d, 1H), 7.74 (s, 1H), 7.34 (d, 1H), 7.26 (d, 1H), 7.04 (d, 1H), 6.75 (d, 1H), 4.36 (d, 1H), 3.50 (m, 2H), 2.57 (m, 2H), 1.82 (d, 1H), 1.70 (m, 1H), 1.32 (m, 3H). MS (ES) for C₂₃H₂₃ClN₆O, found 435.2 (MH+).

### 4-{4-[(2R,65)-2,6-dimethylmorpholin-4-yl]-1H-pyrrolo[2,3-b]pyridin-3-yl}pyrimidin-2-amine

¹H-NMR (400 MHz, CD₃OD): δ 8.24 (d, 1H), 8.10 (d, 1H), 7.73 (s, 1H), 7.04 (d, 1H), 6.74 (d, 1H), 3.75 (m, 2H), 3.30 (m, 2H), 2.34 (t, 3H), 1.02 (d, 6H). MS (ES) for C₁₇H₂₀N₆O, found 325.2 (MH+).

### 4-[3-(2-aminopyrimidin-4-yl)-1H-pyrrolo[2,3-b]pyridin-4-yl]-1-methylpiperazin-2-one

¹H-NMR (400 MHz, CD₃OD): δ 8.18 (d, 1H), 8.15 (d, 1H), 7.84 (s, 1H), 7.08 (d, 1H), 6.78 (d, 1H), 3.65 (s, 2H), 3.41 (m, 4H), 2.96 (s, 3H). MS (ES) for C₁₆H₁₇N₇O, found 324.2 (MH+).

### 4-[4-(3,3-difluoropyrrolidin-1-yl)-1H-pyrrolo[2,3-b]pyridin-3-yl]pyrimidin-2-amine

¹H-NMR (400 MHz, CD₃OD): δ 8.19 (d, 1H), 8.07 (d, 1H), 7.77 (s, 1H), 6.99 (d, 1H), 6.63 (d, 1H), 4.61 (s, 2H), 3.52 (t, 1H), 3.34 (t, 1H), 2.33 (m, 2H). MS (ES) for C₁₅H₁₄F₂N₆, found 317.2 (MH+).

### 1-[3-(2-aminopyrimidin-4-yl)-1H-pyrrolo[2,3-b]pyridin-4-yl]-1,2,3,6-tetrahydropyridin-3-ol

¹H-NMR (400 MHz, CD₃OD): δ 8.17 (d, 1H), 8.13 (d, 1H), 7.89 (s, 1H), 7.21 (d, 1H), 6.85 (d, 1H), 5.83 (d, 1H), 5.71 (d, 1H), 4.38 (s, 1H), 3.62 (m, 1H), 3.46 (m, 2H), 3.06 (m, 1H). MS (ES) for C₁₆H₁₆N₆O, found 309.1 (MH+).

### 2-{1-[3-(2-aminopyrimidin-4-yl)-1H-pyrrolo[2,3-b]pyridin-4-yl]piperidin-4-yl}propan-2-ol

¹H-NMR (400 MHz, CD₃OD): δ 8.21 (d, 1H), 8.07 (d, 1H), 7.74 (s, 1H), 7.09 (d, 1H), 6.77 (d, 1H), 3.55 (d, 2H), 2.59 (m, 2H), 1.66 (m, 2H), 1.39 (m, 3H), 1.16 (d, 6H). MS (ES) for C₁₉H₂₄N₆O, found 353.2 (MH+).

### {1-[3-(2-aminopyrimidin-4-yl)-1H-pyrrolo[2,3-b]pyridin-4-yl]piperidin-4-yl}(4-chlorophenyl)methanone

¹H-NMR (400 MHz, CDCl₃): δ 8.32 (d, 1H), 8.18 (d, 1H), 7.85 (d, 1H), 7.75 (s, 1H), 7.43 (d, 1H), 7.15 (d, 1h), 6.73 (d, 1H), 5.14 (s, 2H), 3.59 (d, 2H), 3.35 (m, 1H), 2.84 (t, 2H), 1.85 (m, 4H). MS (ES) for C₂₃H₂₁ClN₆O, found 433.1 (MH+).

### 4-{4-[3-(3-methyl-1,2,4-oxadiazol-5-yl)pyrrolidin-1-yl]-1H-pyrrolo[2,3-b]pyridin-3-yl}pyrimidin-2-amine

¹H-NMR (400 MHz, CDCl₃): δ 8.21 (d, 1H), 8.05 (d, 1H), 7.62 (s, 1H), 6.93 (d, 1H), 6.52 (d, 1H), 5.17 (s, 2H), 3.70 (m, 2H), 3.59 (m, 1H), 3.45 (m, 1H0, 3.30 (m, 1h), 2.38 (s, 3H), 2.30 (m, 2H). MS (ES) for C₁₈H₁₈N₈O, found 363.2 (MH+).

### {(2S)-1-[3-(2-aminopyrimidin-4-yl)-1H-pyrrolo[2,3-b]pyridin-4-yl]-2,3-dihydro-1H-indol-2-yl}methanol

¹H-NMR (400 MHz, CD₃OD): δ 8.25 (d, 1H), 7.86 (d, 1H) 7.82 (s, 1H), 7.11 (d, 1H), 6.96 (d, 1H), 6.91 (d, 1H), 6.95 (s, 1H), 6.46 (t, 1H), 6.02 (s, 1H), 4.15 (s, 1H), 3.70 (d, 1H), 3.50 (m, 1H), 3.23 (m, 2H). MS (ES) for C₂₀H₁₈N₆O, found 359.2 (MH+).

### 3-(2-aminopyrimidin-4-yl)-N-[(1-ethylpyrrolidin-2-yl)methyl]-1H-pyrrolo[2,3-b]pyridin-4-amine

¹H-NMR (400 MHz, CD₃OD): δ 8.06 (d, 1H), 7.98 (s, 1H), 7.87 (d, 1H), 7.08 (d, 1H), 6.37 (d, 1H), 3.70 (m, 2H), 3.42 (m, 1H), 3.30 (m, 2H), 2.84 (m, 2H), 2.15 (m, 1H), 1.91 (m, 2H), 1.20 (t, 3H). MS (ES) for C₁₈H₂₃N₇, found 338.2 (MH+).

### 2-[3-(2-aminopyrimidin-4-yl)-1H-pyrrolo[2,3-b]pyridin-4-yl]-1,2,3,4-tetrahydroisoquinolin-8-ol

¹H-NMR (400 MHz, DMSO-d₆): δ 12.05 (s, 1H), 9.40 (s, 1H), 8.10 (d, 1H), 7.83 (d, 1H), 7.78 (s, 1H), 6.94 (m, 2H), 6.74 (d, 1H), 6.56 (t, 2H), 6.35 (s, 2H), 4.08 (s, 2H), 3.35 (m, 2H), 2.52 (m, 2H). MS (ES) for C₂₀H₁₈N₆O, found 359.1 (MH+).

### 4-{4-[(2R,5S)-2,5-dimethylpiperazin-1-yl]-1H-pyrrolo[2,3-b]pyridin-3-yl}pyrimidin-2-amine

¹H-NMR (400 MHz, CD₃OD): δ 8.27 (d, 1H), 8.21 (d, 1H), 7.86 (s, 1H), 7.31 (d, 1H), 6.98 (d, 1H), 3.34 (m, 1H), 3.35 (m, 1H), 3.19 (d, 1), 2.90 (m, 2H), 2.40 (m, 1H), 1.17 (d, 3H), 0.99 (d, 3H). MS (ES) for C₁₇H₂₁N₇, found 324.2 (MH+).

### {2-[3-(2-aminopyrimidin-4-yl)-1H-pyrrolo[2,3-b]pyridin-4-yl]-2,3-dihydro-1H-isoindol-1-yl}methanol

¹H-NMR (400 MHz, CD₃OD): δ 8.07 (d, 1H), 8.04 (d, 1H), 7.79 (s, 1H), 7.34 (d, 1H), 7.25 (m, 2H), 7.10 (d, 1H), 7.03 (d, 1H), 6.98 (d, 1H), 5.24 (s, 1H), 4.70 (m, 2H), 3.88 (m, 2H), 1.97 (s, 9H). MS (ES) for C₂₀H₁₈N₆O, found 359.2 (MH+).

### 1R,2R)—N-[3-(2-aminopyrimidin-4-yl)-1H-pyrrolo[2,3-b]pyridin-4-yl]cyclohexane-1,2-diamine

¹H-NMR (400 MHz, CDCl₃): δ 9.80 (d, 1H), 8.10 (d, 1H), 7.87 (d, 1H), 7.66 (s, 1H), 6.90 (d, 1H), 6.20 (d, 1H), 3.19 (m, 1H), 2.82 (m, 1H), 2.32 (d, 1H), 2.18 (d, 1H), 1.78 (m, 1H), 1.38 (m, 3H), 1.13 (m, 1H). MS (ES) for C₁₇H₂₁N₇, found 324.2 (MH+).

### 3-(2-aminopyrimidin-4-yl)-N-(2-methyl-2-pyrrolidin-1-ylpropyl)-1H-pyrrolo[2,3-b]pyridin-4-amine

¹H-NMR (400 MHz, CDCl₃): δ 10.50 (s, 1H), 8.07 (d, 1H), 7.92 (d, 1H), 7.73 (s, 1H), 6.87 (d, 1H), 6.13 (d, 1H), 3.17 (d, 2H), 2.73 (m, 4H), 1.78 (m, 4H), 1.22 (s, 6H). MS (ES) for C₁₉H₂₅N₇, found 352.2 (MH+).

### phenylmethyl[(21R)-2-{[3-(2-aminopyrimidin-4-yl)-1H-pyrrolo[2,3-b]pyridin-4-yl]amino}-3-hydroxypropyl]carbamate

¹H-NMR (400 MHz, CDCl₃): δ 8.08 (d, 1H), 7.39 (m, 2H), 7.36 (m, 5H), 6.93 (d, 1H), 6.50 (d, 1H), 5.13 (s, 2H), 3.91 (m, 2H), 3.68 (m, 3H). MS (ES) for C₂₂H₂₃N₇O₃, found 434.2 (MH+).

### 4-(1-methyl-1H-pyrazol-4-yl)-1H-pyrrolo[2,3-b]pyridine

¹H-NMR (400 MHz, CD₃OD): δ 8.27 (s, 1H), 8.12 (d, 1H), 8.07 (s, 1H), 7.41 (d, 1H), 7.26 (d, 1H), 6.77 (d, 1H), 3.99 (s, 3H). MS (ES) for C₁₁H₁₀N₄, found 199.1 (MH+).

### 4-[2-(methyloxy)Pyridin-4-yl]-1H-pyrrolo[2,3-b]pyridine

¹H-NMR (400 MHz, CDCl₃): δ 9.10 (s, 1H), 8.38 (d, 1H), 8.29 (d, 1H), 7.40 (t, 1H), 7.22 (dd, 2H), 7.09 (s, 1H), 6.70 (q, 1H), 4.01 (s, 3H). MS (ES) for C₁₃H₁₁N₃O, found 226.1 (MH+).

### 4-(6-chloropyridin-3-yl)-1H-pyrrolo[2,3-b]pyridine

¹H-NMR (400 MHz, CD₃OD): δ 8.77 (d, 1H), 8.29 (d, 1H), 8.21 (dd, 1H), 7.63 (d, 1H), 7.51 (d, 1H), 7.26 (d, 1H), 6.66 (d, 1H). MS (ES) for C₁₂H₈ClN₃, found 230.1 (MH+).

### 5-fluoro-4-(1H-pyrazol-4-yl)-1H-pyrrolo[2,3-b]pyridine

¹H-NMR (400 MHz, CD₃OD): δ 8.30 (d, 2H), 8.13 (d, 1H), 7.49 (d, 1H), 6.80 (d, 1H). MS (ES) for C₁₀H₇FN₄, found 203.1 (MH+).

### 4-(1H-pyrrolo[2,3-b]pyridin-4-yl)pyridin-2-amine

¹H-NMR (400 MHz, CD₃OD): δ 8.26 (d, 1H), 8.02 (s, 1H), 7.48 (d, 1H), 7.22 (d, 1H), 6.98 (m, 2H), 6.69 (d, 1H). MS (ES) for C₁₂H₁₀N₄, found 211.0 (MH+).

### 4-(5-chloro-1H-pyrrolo[2,3-b]pyridin-4-yl)pyridin-2-amine

¹H-NMR (400 MHz, CD₃OD): δ 8.27 (s, 1H), 8.03 (t, 1H), 7.44 (d, 1H), 6.73 (d, 2H), 6.39 (m, 1H). MS (ES) for C₁₂H₉ClN₄, found 245.0 (MH+).

### 4-[4-(3-morpholin-4-ylpyrrolidin-1-yl)-1H-pyrrolo[2,3-b]pyridin-3-yl]pyrimidin-2-amine

¹H-NMR (400 MHz, d6-DMSO): δ 11.81 (s, 1H), 8.17-8.15 (d, 1H), 7.95-7.92 (d, 1H), 7.51 (s, 1H), 6.72-6.70 (d, 1H), 6.48 (s, 2H), 6.42-6.41 (d, 1H), 3.54-3.52 (m, 4H), 3.23-3.11 (m, 2H), 2.91-2.85 (t, 1H), 2.78-2.70 (t, 1H), 2.36-2.30 (m, 2H), 2.26-2.19 (m, 2H), 1.99-1.93 (m, 1H), 1.65-1.60 (m, 1H). MS (ES) for C₁₉H₂₃N₇O, found 366.0 (MH+).

### 1-[3-(2-aminopyrimidin-4-yl)-1H-pyrrolo[2,3-b]pyridin-4-yl]-3-phenylpyrrolidin-3-ol

¹H-NMR (400 MHz, d6-DMSO): δ 11.46 (s, 1H), 8.11-8.10 (d, 1H), 7.92-7.91 (d, 1H), 7.45 (s, 1H), 7.35-7.20 (m, 5H), 6.74-6.73 (d, 1H), 6.47-6.42 (s, 2H), 6.42-6.40 (d, 1H), 5.27 (s, 1H), 3.60-3.51 (m, 2H), 3.41-3.39 (m, 2H), 2.19-2.02 (m, 2H). MS (ES) for O₂₁H₂₀N₆O, found 373.0 (MH+).

### 1-[3-(2-aminopyrimidin-4-yl)-1H-pyrrolo[2,3-b]pyridin-4-yl]-3-methylpyrrolidin-3-ol

¹H-NMR (400 MHz, d6-DMSO): δ 11.76 (s, 1H), 8.13-8.12 (d, 1H), 7.91-7.89 (d, 1H), 7.47 (s, 1H), 6.73-6.71 (d, 1H), 6.45 (s, 2H), 6.36-6.35 (d, 1H), 4.68 (s, 1H), 3.31-3.26 (m, 1H), 3.22-3.16 (m, 1H), 3.11-3.03 (m, 2H), 1.77-1.65 (m, 2H). MS (ES) for C₁₆H₁₈N₆O, found 311.0 (MH+).

4-[4-(3-phenylpyrrolidin-1-yl)-1H-pyrrolo[2,3-b]pyridin-3-yl]pyrimidin-2-amine.

¹H-NMR (400 MHz, d6-DMSO): δ 11.81 (s, 1H), 8.14-8.13 (d, 1H), 7.96-7.93 (d, 1H), 7.52 (s, 1H), 7.31-7.27 (m, 2H), 7.22-7.18 (m, 3H), 6.76-6.75 (d, 1H), 6.48-6.46 (m, 3H), 3.60-3.56 (m, 1H), 3.33-3.28 (m, 2H), 3.20-3.15 (m, 2H), 2.25-2.17 (m, 1H), 1.89-1.80 (m, 1H). MS (ES) for C₂₁H₂₀N₆, found 357.0 (MH+).

### 4-{4-[2-(2-thienyl)pyrrolidin-1-yl]-1H-pyrrolo[2,3-b]pyridin-3-yl}pyrimidin-2-amine

¹H-NMR (400 MHz, d6-DMSO): δ 11.85 (s, 1H), 9.59 (m, 1H), 8.11-8.10 (d, 1H), 8.02 (s, 1H), 7.85-7.83 (d, 2H), 7.32-7.31 (d, 1H), 7.10-7.06 (m, 1H), 6.97-6.92 (m, 2H), 6.71-6.67 (m, 1H), 6.58-6.55 (m, 2H), 6.22-6.18 (d, 1H), 6.12-6.04 (m, 1H), 3.44-3.39 (m, 2H), 2.60-2.55 (m, 2H). MS (ES) for C₁₉H₁₈N₆S, found 363.0 (MH+).

### 1-[3-(2-aminopyrimidin-4-yl)-1H-pyrrolo[2,3-b]pyridin-4-yl]piperidin-3-ol

¹H-NMR (400 MHz, DMSO-d₆): δ 12.00 (s, 1H), 8.20 (d, 1H), 8.08 (d, 1H), 7.74 (d, 1H), 6.99 (d, 1H), 6.67 (d, 1H), 6.39 (s, 2H), 4.77 (m, 1H), 3.66-3.42 (m, 2H), 3.18 (m, 1H), 2.56-2.37 (m, 2H), 1.84 (m, 1H), 1.57-1.40 (m, 2H), 1.22-1.11 (m, 1H). MS (ES) for C₁₆H₁₈N₆O, found 311.2 (MH+).

4-[4-(2,7-diazaspiro[4.4]non-2-yl)-1H-pyrrolo[2,3-b]pyridin-3-yl]pyrimidin-2-amine

¹H-NMR (400 MHz, DMSO-d₆): δ 8.15 (d, 1H), 7.95 (d, 1H), 7.55 (s, 1H), 6.76 (d, 1H), 6.47 (s, 2H), 6.42 (d, 1H), 3.19-3.08 (m, 4H), 3.05-2.96 (m, 2H), 2.91-2.81 (m, 2H), 1.83-1.65 (m, 4H). MS (ES) for C₁₈H₂₁N₇, found 336.3 (MH+).

### 2-{(2S)-1-[3-(2-aminopyrimidin-4-yl)-1H-pyrrolo[2,3-b]pyridin-4-yl]pyrrolidin-2-yl}propan-2-ol

¹H-NMR (400 MHz, DMSO-d₆): δ 11.81 (s, 1H), 8.08 (d, 1H), 7.90 (d, 1H), 7.61 (s, 1H), 6.98 (d, 1H), 6.86 (d, 1H), 6.34 (s, 2H), 4.53 (s, 1H), 4.10 (t, 1H), 3.08-2.93 (m, 2H), 1.96-1.77 (m, 2H), 1.63-1.53 (m, 1H), 1.39-1.28 (m, 1H), 1.15 (s, 3H), 1.12 (s, 3H). MS (ES) for C₁₈H₂₂N₆O, found 339.2 (MH+).

### {(3R)-1-[3-(2-aminopyrimidin-4-yl)-1H-pyrrolo[2,3-b]pyridin-4-yl]pyrrolidin-3-yl}methanol

¹H-NMR (400 MHz, DMSO-d₆): δ 11.85 (s, 1H), 8.15 (d, 1H), 7.96 (d, 1H), 7.58 (s, 1H), 6.76, (d, 1H), 6.46 (d, 1H), 6.43 (2s, 2H), 4.65 (t, 1H), 3.33-3.25 (m, 2H), 3.22 (dd, 1H), 3.09 (m, 1H), 3.02 (m, 1H), 2.92 (dd, 1H), 2.28 (m, 1H), 1.82 (m, 1H), 1.48 (m, 1H). MS (ES) for C₁₆H₁₈N₆O, found 311.3 (MH+).

### {1-[3-(2-aminopyrimidin-4-yl)-1H-pyrrolo[2,3-b]pyridin-4-yl]piperidin-2-yl}methanol

¹H-NMR (400 MHz, DMSO-d₆): δ 11.97 (s, 1H), 8.21 (d, 1H), 8.05 (d, 1H), 7.72 (d, 1H), 7.06 (d, 1H), 6.72 (d, 1H), 6.38 (s, 2H), 4.59 (m, 1H), 3.45 (m, 2H), 3.16 (m, 1H), 3.02 (m, 2H), 1.66-1.42 (m, 6H). MS (ES) for C₁₇H₂₀N₆O, found 325.3 (MH+).

### (3R)-1-[3-(2-aminopyrimidin-4-yl)-1H-pyrrolo[2,3-b]pyridin-4-yl]piperidin-3-ol

¹H-NMR (400 MHz, DMSO-d₆): δ 12.00 (s, 1H), 8.20 (d, 1H), 8.08 (d, 1H), 7.74 (d, 1H), 7.00 (d, 1H), 6.67 (d, 1H), 6.40 (s, 2H), 4.77 (d, 1H), 3.62 (m, 1H), 3.41 (br dd, 1H), 3.20 (m, 1H), 2.49-2.35 (m, 2H), 1.85 (m, 1H), 1.59-1.37 (m, 2H), 1.18 (dq, 1H). MS (ES) for C₁₆H₁₈N₆O, found 311.2 (MH+).

### methyl 7-[3-(2-aminopyrimidin-4-yl)-1H-pyrrolo[2,3-b]pyridin-4-yl]-2,7-diazaspiro[4.4]nonane-2-carboxylate

¹H-NMR (400 MHz, DMSO-d₆): δ 11.85 (s, 1H), 8.15 (d, 1H), 7.95 (d, 1H), 7.55 (s, 1H), 6.76 (d, 1H), 6.47 (s, 2H), 6.45 (d, 1H), 3.57 (s, 3H), 3.30-3.07 (m, 8H), 1.91-1.66 (m, 4H). MS (ES) for C₂₀H₂₃N₇O₂, found 394.3 (MH+).

### 7-[3-(2-aminopyrimidin-4-yl)-1H-pyrrolo[2,3-b]pyridin-4-yl]-2,7-diazaspiro[4.4]nonane-2-carboxamide

¹H-NMR (400 MHz, DMSO-d₆): δ 11.85 (s, 1H), 8.17 (d, 1H), 7.95 (d, 1H), 7.55 (d, 1H), 6.77 (d, 1H), 6.47 (s, 2H), 6.45 (d, 1H), 5.71 (s, 2H), 3.29-3.08 (m, 8H), 1.86-1.66 (m, 4H). MS (ES) for C₁₉H₂₂N₈O, found 379.2 (MH+).

### 4-{4-[3-(butyloxy)-3-methylpiperidin-1-yl]-1H-pyrrolo[2,3-b]pyridin-3-yl}pyrimidin-2-amine

¹H-NMR (400 MHz, DMSO-d₆): δ 8.19 (br s, 1H), 8.05 (br s, 1H), 7.77 (s, 1H), 7.14 (d, 1H), 6.79 (br s, 1H), 3.44-3.36 (m, 2H), 2.90-2.77 (m, 2H), 1.83-1.74 (m, 1H), 1.55-1.43 (m, 1H), 1.41-1.16 (m, 8H), 1.13 (s, 3H), 0.84 (t, 3H). MS (ES) for C₂₁H₂₈N₆O, found 381.3 (MH+).

### 2-(1H-pyrrolo[2,3-b]pyridin-3-yl)-1,3-thiazol-4-amine

¹H-NMR (400 MHz, DMSO-d₆): δ 12.61 (s, 1H), 8.46 (dd, 1H), 8.30 (dd, 1H), 8.04 (s, 1H), 7.23 (dd, 1H), 5.75 (s, 1H), 5.35 (s, 2H). MS (ES) for C₁₀H₈N₄S, found 217.2 (MH+).

### {(2R)-1-[3-(2-aminopyrimidin-4-yl)-1H-pyrrolo[2,3-b]pyridin-4-yl]-3,3-difluoropyrrolidin-2-yl}methanol

¹H-NMR (400 MHz, DMSO-d₆): δ 12.93 (s, 1H), 8.31 (d, 1H), 8.19 (d, 1H), 8.12 (s, 1H), 7.23 (d, 1H), 7.03 (d, 1H), 4.31-3.09 (m, 4H), 2.33 (m, 2H). MS (ES) for C₁₆H₁₆F₂N₆O, found 347.1 (MH+).

### 4-(4-{4-[2-(methyloxy)ethyl]piperazin-1-yl}-1H-pyrrolo[2,3-b]pyridin-3-yl)pyrimidin-2-amine

¹H-NMR (400 MHz, CDCl₃): δ 8.29 (d, 1H), 8.13 (d, 1H), 7.76 (s, 1H), 7.17 (d, 2H), 6.70 (d, 1H), 5.13 (br s, 2H), 3.51 (t, 2H), 3.42 (s, 3H), 3.15 (br s, 4H), 2.59 (t, 2H), 2.51 (br s, 4H). MS (ES) for C₁₈H₂₃N₇O, found 354.2 (MH+).

### 2-{4-[3-(2-aminopyrimidin-4-yl)2-1H-pyrrolo[2,3-b]pyridin-4-yl]piperazin-1-yl}ethanol

¹H-NMR (400 MHz, DMSO-d₆): δ 8.31 (d, 1H), 8.07 (d, 1H), 7.72 (s, 1H), 6.98 (d, 1H), 6.67 (d, 1H), 6.38 (s, 2H), 3.48 (t, 2H), 2.92 (br s, 4H), 2.44 (br s, 4H), 2.37 (t, 2H). MS (ES) for C₁₇H₂₁N₇O, found 340.2 (MH+).

### 4-{4-[4-(2-aminoethyl)piperazin-1-yl]-1H-pyrrolo[2,3-b]pyridin-3-yl}pyrimidin-2-amine

¹H-NMR (400 MHz, MeOD-d₄): δ 8.24 (d, 1H), 8.13 (d, 1H), 7.78 (s, 1H), 7.10 (d, 1H), 6.79 (d, 1H), 3.14 (br s, 4H), 3.03 (t, 2H), 2.62 (t, 2H), 2.54 (br s, 4H). MS (ES) for C₁₇H₂₂N₈, found 339.2 (MH+).

### 4-{4-[4-(1-methylethyl)piperazin-1-yl]-1H-pyrrolo[2,3-b]pyridin-3-yl}pyrimidin-2-amine

¹H-NMR (400 MHz, MeOD-d₄): δ 8.27 (d, 1H), 8.16 (d, 1H), 7.82 (s, 1H), 7.18 (d, 1H), 6.82 (d, 1H), 3.28 (m, 5H), 3.07 (m, 4H), 1.28 (m, 6H). MS (ES) for C₁₈H₂₃N₇, found 338.3 (MH+).

### 3-(2-aminopyrimidin-4-yl)-N-(1,4-dioxan-2-ylmethyl)-1H-pyrrolo[2,3-b]pyridin-4-amine

¹H-NMR (400 MHz, DMSO-d₆): δ 10.03 (br s, 1H), 8.18 (m, 2H), 7.81 (m, 1H, 7.11 (d, 1H), 6.48 (br s, 1H), 6.20 (m, 1H), 4.18-4.09 (m, 7H), 3.25 (d, 2H). MS (ES) for C₁₆H₁₈N₆O₂, found 327.2 (MH+).

### 3-(2-aminopyrimidin-4-yl)-N-(morpholin-2-ylmethyl)-1H-pyrrolo[2,3-b]pyridin-4-amine

¹H-NMR (400 MHz, CDCl₃): δ 8.28 (d, 1H), 7.72 (m, 2H), 6.94 (d, 2H), 6.21 (d, 1H), 5.88 (br s, 2H), 3.97-3.85 (m, 2H), 3.21-2.97 (m, 2H), 2.93-2.89 (m, 3H), 2.83-2.76 (m, 2H). MS (ES) for C₁₆H₁₉N₇O, found 326.2 (MH+).

### 2-{[3-(2-aminopyrimidin-4-yl)-1H-pyrrolo[2,3-b]pyridin-4-yl]amino}ethanol

¹H-NMR (400 MHz, DMSO-d₆): δ 10.27 (m, 1H), 8.09 (m, 2H), 7.81 (d, 1H), 7.08 (d, 1H), 6.83 (m, 2H), 6.12 (d, 1H), 5.64 (m, 1H), 3.79 (m, 2H), 3.21 (m, 2H). MS (ES) for C₁₃H₁₄N₆O, found 271.2 (MH+).

### 1,1-dimethylethyl N-[3-(2-aminopyrimidin-4-yl)-1H-pyrrolo[2,3-b]pyridin-4-yl]glycinate

¹H-NMR (400 MHz, MeOD-d₄): δ 8.48 (m, 1H), 8.28 (d, 1H), 8.03 (m, 1H), 7.42 (m, 1H), 6.63 (m, 1H), 4.48 (s, 2H), 1.57 (s, 9H). MS (ES) for C₁₇H₂₀N₆O₂, found 341.4 (MH+).

### 3-(2-aminopyrimidin-4-yl)-N-[2-(methyloxy)ethyl]-1H-pyrrolo[2,3-b]pyridin-4-amine

¹H-NMR (400 MHz, CDCl₃): δ 8.28 (d, 1H), 7.89 (m, 2H), 7.72 (m, 1H), 6.92 (d, 1H), 6.20 (m, 1H), 3.91 (m, 2H), 3.51-3.46 (m, 5H). MS (ES) for C₁₄H₁₆N₆O, found 285.1 (MH+).

### 4-(1H-pyrrolo[2,3-b]pyridin-3-yl)pyrimidin-2-amine

¹H-NMR (400 MHz, MeOD-d₄): δ 8.93 (m, 1H), 8.28 (m, 1H), 8.20 (s, 1H), 8.13 (d, 1H), 7.25 (dd, 1H), 7.07 (d, 1H), 4.62 (br s, 2H). MS (ES) for C₁₁H₉N₆, found 212.1 (MH+).

### N˜2˜-[3-(2-aminopyrimidin-4-yl)-1H-pyrrolo[2,3-b]pyridin-4-yl]-N,N-dimethylglycinamide

¹H-NMR (400 MHz, DMSO-d₆): δ 10.22 (br s, 1H), 8.17 (s, 1H), 8.04 (d, 1H), 7.82 (d, 1H), 7.10 (d, 1H), 6.21 (d, 1H), 4.06 (m, 2H), 3.04 (s, 3H), 2.97 (s, 3H). MS (ES) for C₁₆H₁₇N₇O, found 312.1 (MH+).

### Example 79

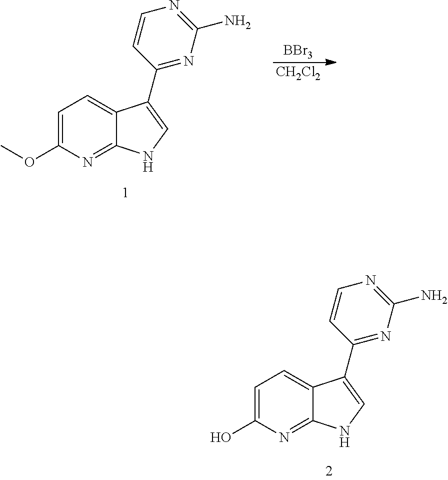

### 3-(2-aminopyrimidin-4-yl)-1H-pyrrolo[2,3-b]pyridin-6-ol

To a solution of 1^{ref} (100 mg, 0.41 mmol) in CH₂Cl₂ (15 mL) at −40° C. was added a solution of BBr₃ in CH₂Cl₂ (1M, 5 mL) and the solution was allowed to warm to room temperature while stirring for 1 h. A solution of saturated sodium bicarbonate (10 mL) was added to the reaction and the layers were separated. The aqueous layer was extracted with CH₂Cl₂ (3×10 mL). The combined organic layers were washed with brine, dried over anhydrous sodium sulfate, filtered and concentrated to yield a brown residue. This residue was purified via reverse phase HPLC to yield the desired compound (15.2 mg, 0.06 mmol) in 16.3% yield.

¹H-NMR (400 MHz, MeOD-d₄): δ 8.72 (d, 1H), 8.18 (d, 1H), 7.73 (s, 1H), 6.98 (d, 1H), 6.43 (d, 1H), MS (ES) for C₁₁H₉N₆O, found 228.1 (MH+).

Reference: Synthesis of pyrrolo[2,3-b]pyridine compounds, azaindole compounds used in the synthesis of these compounds, their fabrication processes, and their uses. Meijer, Laurent; Joseph, Benoit; Liger, Francois; Marquet, Bernard. (Centre National de la Recherche Scientifique —CNRS, Fr.). Fr. Demande (2008), 43 pp. CODEN: FRXXBL FR 2912744 A120080822 patent written in French. Application: FR 2007-1138 20070216. Priority: CAN 149:288893 AN 2008:1013719 CAPLUS

Using analogous synthetic techniques as in Example 54, and substituting with the appropriate alternative starting materials, the following compounds were prepared. Alternative starting materials were obtained commercially unless otherwise indicated.

### 24-{-4-[(3S)-3-(methyloxy)pyrrolidin-1-yl]-1H-pyrrolo[2,3-b]pyridin-3-yl}pyrimidin-2-amine

¹H-NMR (400 MHz, DMSO-d₆) δ 8.17 (d, 1H), 7.99 (d, 1H), 7.78 (s, 1H), 6.77 (d, 1H), 6.42 (m, 2H), 3.98 (m, 1H) 3.34-3.07 (m, 7H), 1.98-1.82 (m, 2H). MS (ES) for C₁₆H₁₈N₆O, found 311.2 (MH+).

### 4-{4-[(3R)-3-(methyloxy)pyrrolidin-1-yl]-1H-pyrrolo[2,3-b]pyridin-3-yl}pyrimidin-2-amine

¹H-NMR (400 MHz, DMSO-d₆): δ 8.17 (d, 1H), 7.99 (d, 1H), 7.78 (s, 1H), 6.77 (d, 1H), 6.42 (m, 2H), 3.98 (m, 1H) 3.34-3.07 (m, 7H), 1.98-1.82 (m, 2H). MS (ES) for C₁₈H₂₃N₆O, found 311.2 (MH+).

### 2-{(25)-1-[3-(2-aminopyrimidin-4-O-1H-pyrrolo[2,3-b]pyridin-4-yl]pyrrolidin-2-yl}ethanol

¹H-NMR (400 MHz, DMSO-d₆): δ 8.17 (m, 1H), 7.96 (d, 1H), 7.61 (s, 1H), 6.79 (d, 1H), 6.56 (d, 1H), 6.39 (br s, 2H), 3.8 (m, 2H), 3.22 (m, 3H), 2.83 (m, 2H), 1.67-1.41 (m, 4H). MS (ES) for C₁₇H₂₁N₆O, found 325.2 (MH+).

### (2R,3S)-1-[3-(2-aminopyrimidin-4-yl)-1H-pyrrolo[2,3-b]pyridin-4-yl]-2-(hydroxymethyl)pyrrolidin-3-ol

¹H-NMR (400 MHz, DMSO-d₆): δ 8.17 (d, 1H), 8.03 (d, 1H), 7.65 (d, 1H), 6.82 (d, 1H), 6.63 (d, 1H), 6.31 (s, 2H), 4.98 (m, 1H), 4.21 (s, 1H), 3.85 (m, 2H), 2.82 (m 2H), 1.87 (m, 2H). MS (ES) for C₁₆H₁₈N₆O₂, found 327.2 (MH+).

### {1-[3-(2-aminopyrimidin-4-yl)-1H-pyrrolo[2,3-b]pyridin-4-yl]pyrrolidin-3-yl}methanol

¹H-NMR (400 MHz, DMSO-d₆): δ 11.8 (S, 1H), 8.1 (d, 1H), 7.9 (d, 1H), 7.6 (s, 1H), 6.8 (d, 1H), 6.20 (m, 3H), 4.6 (m, 1H), 3.3 (m, 1H), 3.10 (m, 1H), 3.0 (m, 2H), 2.9 (m, 1H), 2.5 (m, 1H), 2.3 (m, 1H) 1.8 (m, 1H), 1.5 (m, 1H).

MS (ES) for C₁₆H₁₈N₆O. found 311.0 (MH+).

### 3-{[3-(2-aminopyrimidin-4-yl)-1H-pyrrolo[2,3-b]pyridin-4-yl]amino}propane-1,2-diol

¹H-NMR (400 MHz, DMSO-d₆): δ 13.2 (s, 1H), 11.5 (s, 1H), 8.6 (s, 1H), 8.3 (s, 1H), 8.1 (s, 1H),7.9 (br, 2H), 7.4 (s, 1H), 6.6 (s, 1H), 3.2-3.9 (m, 3H).

MS (ES) for C₁₄H₁₆N₆O₂. found 301.0 (MH+).

### {(3S)-1-[3-(2-aminopyrimidin-4-yl)-1H-pyrrolo[2,3-b]pyridin-4-yl]pyrrolidin-3-yl}methanol

¹H-NMR (400 MHz, DMSO-d₆): δ 11.9 (S, 1H), 8.2 (d, 1H), 7.9 (d, 1H), 7.6 (s, 1H), 6.8 (d, 1H), 6.20 (m, 3H), 4.6 (m, 1H), 3.2 (m, 3H), 3.0 (m, 3H), 2.3 (m, 1H), 1.9 (m, 1H), 1.4 (m, 1H).

MS (ES) for C₁₆H₁₈N₆O. found 311.0 (MH+).

### (3S)-1-[3-(2-aminopyrimidin-4-yl)-1H-pyrrolo[2,3-b]pyridin-4-yl]piperidin-3-ol

¹H-NMR (400 MHz, DMSO-d₆): δ 12.0 (s, 1H), 8.2 (d, 1H), 8.1 (d, 1H), 7.8 (s, 1H), 7.0 (d, 1H), 6.7 (d, 1H), 6.4 (s, 2H), 4.8 (d, 1H), 3.6 (m, 1H), 3.2 (m, 2H), 2.4 (m, 2H), 1.8 (m, 1H), 1.5 (m, 2H), 1.2 (m, 1H).

MS (ES) for C₁₆H₁₈N₆O. found 311.0 (MH+).

### {(2S)-1-[3-(2-aminopyrimidin-4-yl)-1H-pyrrolo[2,3-b]pyridin-4-yl]pyrrolidin-2-yl}methyl L-valinate

¹H-NMR (400 MHz, DMSO-d₆): δ 8.1 (d, 1H), 8.0 (d, 1H), 7.6 (s, 1H), 6.8 (d, 1H), 6.7 (d, 1H), 6.4 (s, 2H), 4.2 (m, 3H), 3.4 (m, 2H), 3.0 (m, 2H), 2.1 (s, 1H), 1.8 (m, 4H), 0.8 (d, 3H), 0.7 (d, 3H).

MS (ES) for O₂₁H₂₇N₇O₂. found 410 (MH+).

### {(2S)-1-[3-(6-aminopyrimidin-4-yl)-1H-pyrrolo[2,3-b]pyridin-4-yl]pyrrolidin-2-yl}methanol

¹H-NMR (400 MHz, DMSO-d₆): δ 11.9 (s, 1H), 8.3 (s, 1H), 8.0 (d, 1H), 7.6 (s, 1H), 6.7 (m, 4H), 3.8 (m, 1H), 3.4 (m, 2H), 3.2 (m, 1H), 2.9 (m, 1H), 2.0 (m, 1H), 1.8 (m, 1H), 1.6 (m, 2H).

MS (ES) for C₁₆H₁₈N₆O. found 311 (MH+).

### 2-[3-(2-aminopyrimidin-4-O-1H-pyrrolo[2,3-b]pyridin-4-yl]hexahydrocyclopenta[c]pyrrol-5(1H)-one oxime

¹H-NMR (400 MHz, DMSO-d₆): δ 8.4 (s, 1H), 8.1 (d, 1H), 8.0 (d, 1H), 6.8 (d, 1H), 6.4 (s, 1H), 6.4 (br, 2H), 3.2 (m, 2H), 3.0 (m, 2H), 2.8 (m, 2H), 2.2 (m, 2H).

MS (ES) for C₁₈H₁₉N₇O. found 350 (MH+).

### 4-(4-chloro-1H-pyrrolo[2,3-b]pyridin-3-yl)-1,3-thiazol-2-amine

¹H-NMR (400 MHz, DMSO-d₆): δ 12.2 (s, 1H), 8.2 (d, 1H), 7.7 (s, 1H), 7.2 (d, 1H), 6.9 (br, 1H), 6.6 (s, 1H).

MS (ES) for C₁₀H₇ClN₄S. found 251 (MH+).

### 4-(1H-pyrrolo[2,3-b]pyridin-4-yl)pyrimidin-2-amine

¹H-NMR (400 MHz, DMSO-d₆): δ 12.0 (s, 1H), 8.4 (m, 2H), 7.6 (m, 2H), 7.3 (s, 1H), 7.1 (s, 1H), 6.8 (br, 2H).

MS (ES) for C₁₁H₉N₅. found 212 (MH+).

### 4-(1H-pyrazol-4-yl)-1H-pyrrolo[2,3-b]pyridine

¹H-NMR (400 MHz, DMSO-d₆): 13.2 (br, 1H), 11.6 (s, 1H), 8.5 (s, 1H), 8.2 (m, 2H), 7.6 (m, 1H), 7.4 (d, 1H), 6.8 (br, 1H).

MS (ES) for C₁₀H₈N₄. found 185 (MH+).

### 4-pyridin-4-yl-1H-pyrrolo[2,3-b]pyridine

¹H-NMR (400 MHz, DMSO-d₆): 12.0 (br, 1H), 8.8 (d, 2H), 8.4 (d, 1H), 7.8 (d, 2H), 7.6 (d, 1H), 7.4 (d, 1H), 6.7 (d, 1H).

MS (ES) for C₁₂H₉N₃. found 196 (MH+).

### Example 80

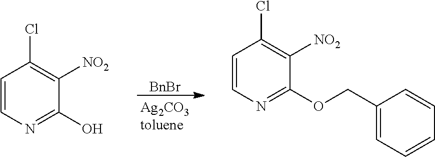

### 2-(Benzyloxy)-4-chloro-3-nitropyridine

4-Chloro-3-nitropyridin-2-ol (9.3518 g, 53.58 mmol, 1 equivalent) was weighed out and added to a flask with magnetic stir bar followed by silver carbonate (17.291 g, 62.7 mmol, 1.17 equivalents). Toluene (105 mL) was added, then benzyl bromide (7.46 mL, 62.7 mmol, 1.17 equivalents), and the suspension was stirred overnight at room temperature.

Solids were removed by filtration, and the toluene filtrate was concentrated and purified by silica chromatography in hexane/ethyl acetate. 9.371 g of the pure title compound was obtained (49% yield).

¹H-NMR (400 MHz, DMSO-d₆): δ 8.40 (d, 1H), 7.50 (d, 1H), 7.42-7.37 (m, 4H), 7.36-7.33 (m, 1H), 5.51 (s, 2H).

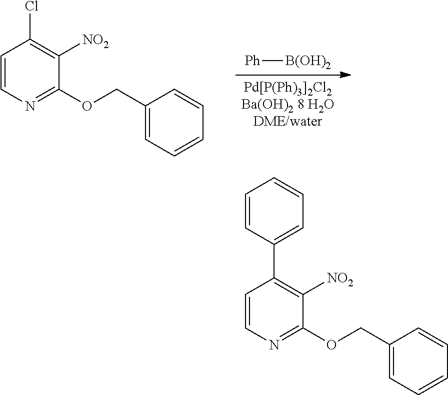

### 2-(Benzyloxy)-3-nitro-4-phenylpyridine

2-(Benzyloxy)-4-chloro-3-nitropyridine (4.0038 g, 15.13 mmol, 1 equivalent) was weighed out and added to a reaction tube with magnetic stir bar. This was followed by phenylboronic acid (3.6936 g, 30.2 mmol, 2 equivalents) and barium hydroxide octahydrate (14.3172 g, 45.39 mmol, 3 equivalents). Dimethoxyethane (42 mL) and water (4.2 mL) were added. The solution was subjected to vigorous subsurface nitrogen sparge and bis(triphenylphosphine) palladium dichloride (1.0657 g, 1.513 mmol, 10 mol %) was weighed out and added. The reaction tube was sealed under nitrogen, and heated at 85° C. for three hours. After that time, the reaction was diluted into ethyl acetate and washed twice with saturated aqueous sodium bicarbonate, twice with brine, and dried over sodium sulfate. The product solution was filtered, concentrated, and purified by silica chromatography in 25% dichloromethane in hexane. 3.391 g of the pure title compound were obtained after drying (73% yield).

¹H-NMR (400 MHz, DMSO-d₆): δ 8.46 (d, 1H), 7.54-7.52 (m, 3H), 7.45-7.35 (m, 7H), 7.30 (d, 1H), 5.55 (s, 2H). MS (ES) for C₁₈H₁₄N₂O₃, found 307.0 (MH+).

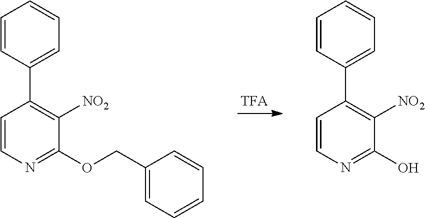

### 3-Nitro-4-phenylpyridin-2-ol

2-(Benzyloxy)-3-nitro-4-phenylpyridine was concentrated in the reaction flask and dried on the high vacuum (3.391 g, 11.07 mmol, 1 equivalent). The material was cooled to zero degrees C. and a magnetic stir bar was added. Trifluoroacetic acid (100 mL) was added carefully and the reaction was stirred at room temperature overnight.

The reaction was concentrated to remove TFA, chased with hexane, and dried on the high vacuum. The product was used without further purification.

¹H-NMR (400 MHz, DMSO-d₆): δ 7.75 (d, 1H), 7.56-7.49 (m, 3H), 7.46-7.35 (m, 3H), 6.39 (d, 1H). MS (ES) for C₁₁H₈N₂O₃, found 217.0 (MH+).

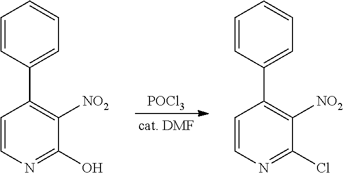

### 2-Chloro-3-nitro-4-phenyl pyridine

3-Nitro-4-phenylpyridin-2-ol was concentrated in the tared reaction flask, dried under high vacuum and weighed (1.082 g, 5.0 mmol, 1 equivalent). Phosphorous oxychloride (33 mL) was added along with a magnetic stir bar, followed by 5 drops of dimethylformamide. The reaction was heated at reflux overnight.

The reaction solution was concentrated to a small volume, and the residue was quenched with saturated aqueous sodium bicarbonate. This was extracted three times with ethyl acetate, and the combined organic layers were washed with brine, dried over sodium sulfate, filtered, concentrated. It was then purified by silica chromatography in hexane/ethyl acetate. After drying, 0.896 g was obtained of the pure title compound (77% yield).

¹H-NMR (400 MHz, DMSO-d₆): δ 8.74 (d, 1H), 7.80 (d, 1H), 7.61-7.53 (m, 3H), 7.49-7.43 (m, 2H). MS (ES) for C₁₁H₇ClN₂O₂, found 235.0 (MH+).

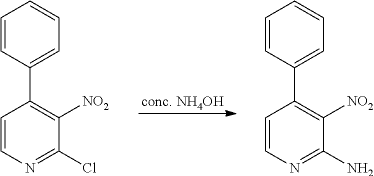

### 3-Nitro-4-phenylpyridin-2-amine

2-Chloro-3-nitro-4-phenylpyridine (0.846 g, 3.61 mmol, 1 equivalent) was transferred to a large reaction tube with magnetic stir bar. Concentrated ammonium hydroxide solution (200 mL) was added, and the tube was sealed under nitrogen and heated at 100° C. overnight.

The reaction solution was allowed to cool-first down to room temperature and then in an ice bath. Precipitated product is filtered with cold water washings and then dried on the high vacuum. 0.566 g of the pure title compound is obtained (73% yield).

¹H-NMR (400 MHz, DMSO-d₆): δ 8.22 (d, 1H), 7.50-7.41 (m, 3H), 7.36-7.29 (m, 2H), 7.04 (s, 2H), 6.55 (d, 1H). MS (ES) for C₁₁H₉N₃O₂, found 216.0 (MH+).

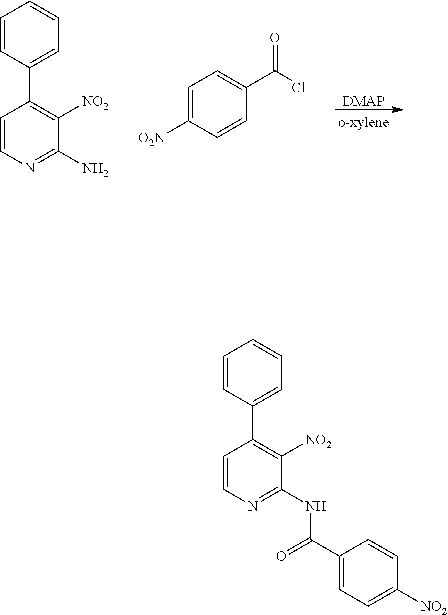

### 4-Nitro-N-(3-nitro-4-phenylpyridin-2-yl)benzamide

3-Nitro-4-phenylpyridin-2-amine (0.566 g, 2.63 mmol, 1 equivalent) was weighed out and added to the reaction flask with magnetic stir bar. O-xylene was added, and 4-dimethylaminopyridine (0.3872 g, 3.17 mmol, 1.2 equivalents) and 4-nitrobenzoyl chloride (0.5873 g, 3.16 mmol, 1.2 equivalents) were weighed out and added. The reaction was heated at 130° C., under nitrogen, for six hours. After that time, the reaction mixture was diluted into ethyl acetate, washed with saturated aqueous sodium bicarbonate, and dried over sodium sulfate. Product solution was filtered, concentrated, and purified by silica chromatography. After drying, 0.691 g of the pure title compound was obtained (72% yield).

¹H-NMR (400 MHz, DMSO-d₆): δ 11.75 (s, 1H), 8.78 (d, 1H), 8.38 (d, 2H), 8.20 (d, 2H), 7.59 (d, 1H), 7.56-7.50 (m, 3H), 7.45-7.38 (m, 2H). MS (ES) for C₁₈H₁₂N₄O₆, found 363.1 (M−H).

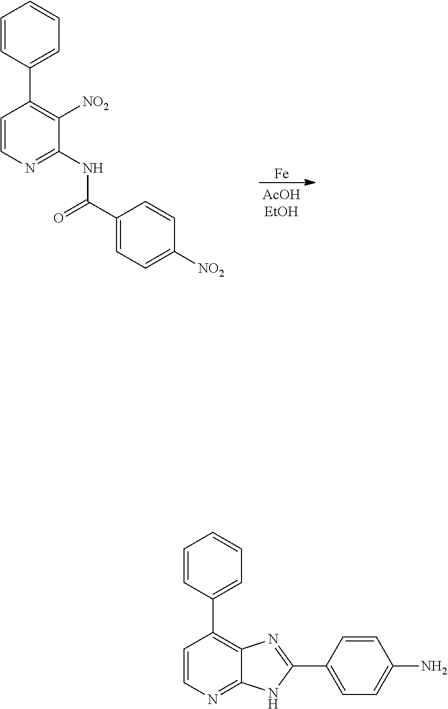

### 4-(7-phenyl-3H-imidazo[4,5-b]pyridin-2-yl)aniline

4-Nitro-N-(3-nitro-4-phenylpyridin-2-yl)benzamide was concentrated in the tared reaction flask, dried on the high vacuum and weighed (0.691 g, 1.90 mmol, 1 equivalent). Ethanol (58 mL) was added along with a magnetic stir bar. Iron (1.0968 g, 19.64 mmol, 10.34 equivalents) was weighed out and added, and acetic acid (2.9 mL, 50.66 mmol, 26.66 equivalents) was added and the reaction was heated at reflux overnight under nitrogen, protected from light.

The reaction was filtered, and then the filtrate was concentrated to dryness. The residue was treated with water, a yellow solid crashed out and this was filtered with water washings. The precipitate (including material caught in the filter) was dissolved in methanol/dichloromethane and concentrated onto a silica plug. This was loaded on a column and eluted, running from 100% dichloromethane up to 5% methanol in dichloromethane solution. After drying, 0.1068 g of the pure title compound was obtained (20% yield).

¹H-NMR (400 MHz, DMSO-d₆): δ 13.18 (s, 1H), 8.38 (d, 2H), 8.24 (d, 1H), 7.96 (d, 2H), 7.57 (t, 2H), 7.47 (m, 2H), 6.68 (d, 2H), 5.73 (s, 2H). MS (ES) for C₁₈H₁₄N₄, found 287.0 (MH+).

### Example 81

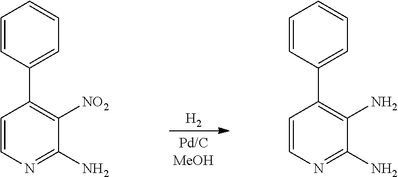

### 4-Phenylpyridine-2,3-diamine

3-Nitro-4-phenylpyridin-2-amine (0.2511 g, 1.167 mmol, 1 equivalent) was weighed out and added to the reaction flask with magnetic stir bar. Methanol (12 mL) was added and the system was put under nitrogen. 10% Palladium on carbon (50 wt % water wet) catalyst (0.0828 g, 0.039 mmol, 3.33 mol %) was weighed out and added, and the system was flushed with nitrogen again. This was hydrogenated under a balloon, stirring two hours at room temperature. This was filtered through Celite with methanol washings, and the filtrate was concentrated and dried on the high vacuum. 0.181 g of the title compound was obtained and used without further purification (84% yield).

¹H-NMR (400 MHz, DMSO-d₆): δ 7.51-7.44 (m, 2H), 7.44-7.37 (m, 3H), 7.35 (d, 1H), 6.32 (d, 1H), 5.55 (s, 2H), 4.35 (s, 2H). MS (ES) for C₁₁H₁₁N₃, found 186.1 (MH+).

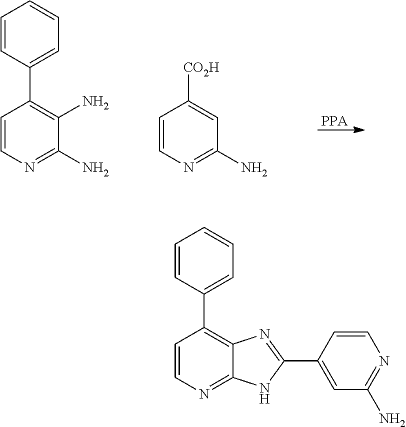

### 4-(7-phenyl-3H-imidazo[4,5-b]pyridin-2-yl)pyridin-2-amine

4-Phenylpyridine-2,3-diamine (0.0611 g, 0.33 mmol, 1 equivalent) was weighed out and added to a reaction tube with magnetic stir bar. 2-Aminoisonicotinic acid (0.0460 g, 0.33 mmol, 1 equivalent) was then weighed out and added. The reaction tube was tared and some polyphosphoric acid (3.62 g) was allowed to ooze into it. This weight was determined by difference, and the tube was sealed under nitrogen and heated at 204° C. for three hours. It was allowed to cool to room temperature, dissolved in water, and neutralized with saturated aqueous sodium bicarbonate. This was extracted four times with ethyl acetate. The combined organic layer was dried over sodium sulfate, filtered, and concentrated.

Product was purified on reverse phase by preparative HPLC. 0.0420 g of the pure title compound was obtained after drying (44% yield).

¹H-NMR (400 MHz, DMSO-d₆): δ 8.42 (d, 1H), 8.31 (br d, 2H), 8.09 (dd, 1H), 7.59 (t, 2H), 7.55 (d, 1H), 7.51 (t, 1H), 7.31 (s, 1H), 7.28 (dd, 1H), 6.23 (s, 2H). MS (ES) for C₁₇H₁₃N₆, found 288.0 (MH+).

### Example 82

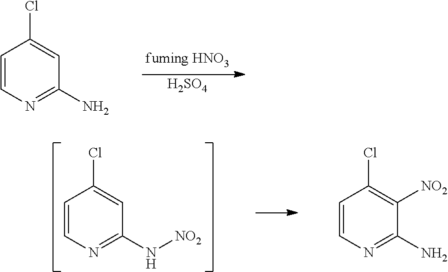

### 4-Chloro-3-nitropyridin-2-amine

4-Chloropyridin-2-amine (15.1204 g, 117.61 mmol, 1 equivalent) was weighed out and added to a flask with magnetic stir bar. The solid was cooled to 0° C. and concentrated sulfuric acid (87.0 mL) was added slowly, in portions. A small pressure-equalizing addition funnel was added and the system was put under nitrogen. The funnel was charged with 4.4 mL of 90% red fuming nitric acid, and this was added in dropwise, slowly, at zero degrees. After addition was complete, the reaction was stirred overnight, slowly warming to room temperature.

A drop of the reaction added to water dissolved totally, indicating completion (the N-nitro intermediate crashes out if it remains). The reaction was quenched by pouring it into ice, then basifying with concentrated ammonium hydroxide. The yellow solid product was filtered, washing with ice water, and dried on the high vacuum.

The solid was extracted several times with ethyl acetate, and the combined extracts were concentrated down. To remove the 5-nitro impurity, it proved necessary to purify this by silica chromatography in 2:1:1 hexane/ethyl acetate/dichloromethane. Two column runs were required to obtain 6.443 g total of the pure title compound (32% yield).

¹H-NMR (400 MHz, DMSO-d₆): δ 8.12 (d, 1H), 7.26 (s, 2H), 6.87 (d, 1H). MS (ES) for C₆H₄ClN₃O₂, found 174.0 (MH+).

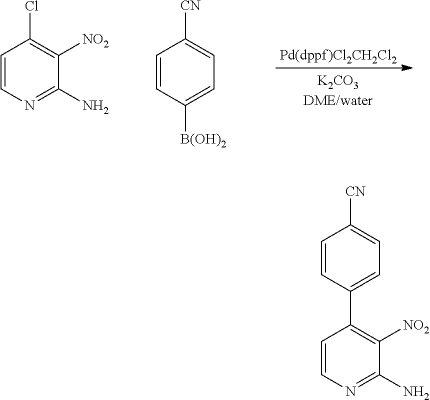

### 4-(2-Amino-3-nitropyridin-4-yl)benzonitrile

4-Chloro-3-nitropyridin-2-amine (3.0013 g, 17.29 mmol, 1 equivalent) was added as a solution in dimethoxyethane (32.0 mL) to a reaction tube with magnetic stir bar. 3.5 mL water was added. 4-Cyanophenylboronic acid (2.7972 g, 19.04 mmol, 1.1 equivalents), potassium carbonate (7.1692 g, 51.87 mmol, 3.0 equivalents) and palladium diphenylphosphinoferrocene dichloride, mono dichloromethane adduct (0.7078 g, 0.867 mmol, 5 mol %) were weighed out and added. The solution was vigorously sparged with nitrogen, sealed under nitrogen, and heated at 90° C. for two hours. This was filtered through Celite with excess methanol/ethyl acetate washings and concentrated onto a silica plug. It was loaded on a column and eluted, running from 50/50 hexane/ethyl acetate up to 100% ethyl acetate. Column product was further purified by trituration with ethyl acetate to give 3.187 g of the pure title compound (77% yield).

¹H-NMR (400 MHz, DMSO-d₆): δ 8.30 (d, 1H), 7.94 (d, 2H), 7.55 (d, 2H), 7.35 (s, 2H), 6.65 (d, 1H). MS (ES) for C₁₂H₈N₄O₂, found 241.0 (MH+).

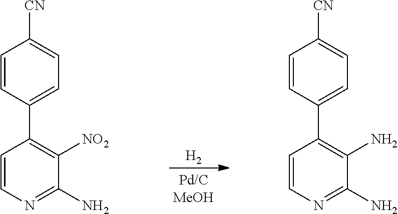

### 4-(2,3-Diaminopyridin-4-yl)benzonitrile

4-(2-Amino-3-nitropyridin-4-yl)benzonitrile (2.55 g, 10.62 mmol, 1 equivalent) was weighed out and added to a flask with magnetic stir bar. This was suspended in 150 mL methanol, and the system was put under nitrogen. 10% Palladium on carbon (50 wt % water wet) catalyst (0.6592 g, 0.310 mmol, 2.9 mol %) was weighed out and added, and the system was flushed with nitrogen again. This was hydrogenated under a balloon, stirring two hours at room temperature. This was filtered through Celite with methanol washings, and the filtrate was concentrated and dried on the high vacuum. 2.213 g of the title compound was obtained and used without further purification (99% yield).

¹H-NMR (400 MHz, DMSO-d₆): 7.90 (d, 2H), 7.60 (d, 2H), 7.34 (d, 1H), 6.30 (d, 1H), 5.65 (s, 2H), 4.54 (s, 2H). MS (ES) for C₁₂H₁₀N₄, found 211.1 (MH+).

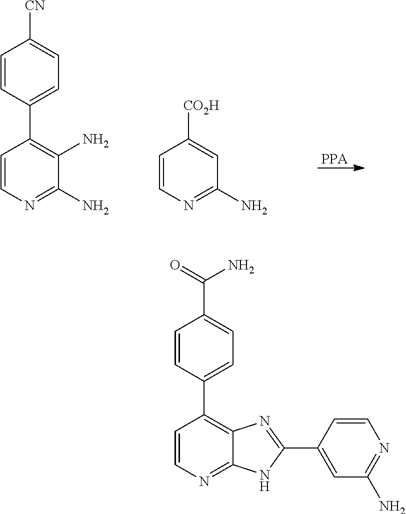

### 4-[2-(2-aminopyridin-4-yl)-3H-imidazo[4,5-b]pyridin-7-yl]benzamide

4-(2,3-Diaminopyridin-4-yl)benzonitrile (0.5016 g, 2.39 mmol, 1 equivalent) was weighed out and added to a reaction tube with magnetic stir bar. 2-Aminoisonicotinic acid (0.3306 g, 2.39 mmol, 1 equivalent) was then weighed out and added. The reaction tube was tared and polyphosphoric acid (8.91 g) was allowed to ooze into it. This weight was determined by difference, and the tube was sealed under nitrogen and heated at 200° C. for two hours. It was then allowed to cool to room temperature before opening. The mixture was diluted with water, and solid precipitate crashed out. This material was collected with water washings, dried, and extracted several times with a 7M solution of ammonia in methanol. The combined methanolic ammonia extract was filtered and concentrated. The residue was purified on reverse phase by preparative HPLC. After drying, 0.0515 g of the pure title compound was obtained (7% yield).

¹H-NMR (400 MHz, DMSO-d₆): 8.41 (d, 1H), 8.36 (br d, 2H), 8.10 (s, 1H), 8.07 (d, 1H), 8.03 (d, 2H), 7.58 (d, 1H), 7.47 (s, 1H), 7.28 (s, 1H), 7.26 (d, 1H), 6.20 (s, 2H). MS (ES) for C₁₈H₁₄N₆O, found 331.3 (MH+).

Using analogous synthetic techniques as in Example 54, and substituting with the appropriate alternative starting materials, the following compounds were prepared. Alternative starting materials were obtained commercially unless otherwise indicated.

### 5-(7-phenyl-3H-imidazo[4,5-b]pyridin-2-yl)pyridin-2-amine

¹H-NMR (400 MHz, DMSO-d₆): δ 8.82 (d, 1H), 8.34 (br s, 2H), 8.29 (d, 1H), 8.19 (dd, 1H), 7.57 (t, 2H), 7.48 (m, 2H), 6.59 (s, 2H), 6.57 (d, 1H). MS (ES) for C₁₇H₁₃N₅, found 288.0 (MH+).

### 2-(6-chloropyridin-34)-7-phenyl-3H-imidazo[4,5-b]pyridine

¹H-NMR (400 MHz, DMSO-d₆): δ 13.96 (s, 1H), 9.26 (d, 1H), 8.65 (dd, 1H), 8.44 (d, 1H), 8.36 (br s, 2H), 7.78 (d, 1H), 7.60 (m, 3H), 7.51 (t, 1H). MS (ES) for C₁₇H₁₁ClN₄, found 307.0 (MH+).

### 4-(4-chloro-1H-pyrrolo[2,3-b]pyridin-2-yl)pyridin-2-amine

¹H-NMR (400 MHz, DMSO-d₆): δ 12.75 (s, 1H), 8.34 (d, 1H), 8.12 (d, 1H), 7.36 (d, 1H), 7.19 (dd, 1H), 7.11 (s, 1H), 7.08 (s, 1H), 6.14 (s, 2H). MS (ES) for C₁₂H₉ClN₄, found 245.1 (MH+).

### (3-(4-(2-aminopyridin-4-yl)-1H-pyrrolo[2,3-b]pyridin-2-yl)phenyl)methanol

¹H-NMR (400 MHz, DMSO-d₆): δ 12.37 (s, 1H), 8.29 (d, 1H), 8.07 (d, 1H), 7.94 (s, 1H), 7.86 (d, 1H), 7.44 (t, 1H), 7.34 (d, 1H), 7.19 (d, 1H), 7.09 (s, 1H), 6.90 (m, 2H), 6.12 (s, 2H), 4.58 (s, 2H). MS (ES) for C₁₉H₁₆N₄O, found 317.2 (MH+).

### 4-[4-(1H-indol-4-yl)-1H-pyrrolo[2,3-b]pyridin-2-yl]pyridin-2-amine

¹H-NMR (400 MHz, DMSO-d₆): δ 12.35 (s, 1H), 11.37 (s, 1H), 8.36 (d, 1H), 7.96 (d, 1H), 7.53 (d, 1H), 7.46 (t, 1H), 7.33-7.26 (m, 3H), 7.03 (dd, 1H), 6.89 (s, 1H), 6.87 (d, 1H), 6.46 (s, 1H), 5.95 (s, 2H). MS (ES) for C₂₀H₁₅N₅, found 326.2 (MH+).

### Example 83

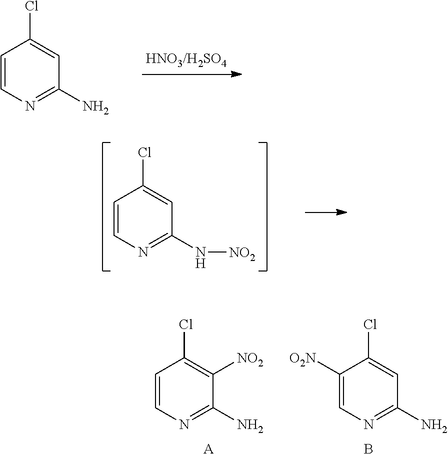

### 4-Chloro-3-nitro-pyridin-2-amine (A)

2-Amino-4-chloropyridine (20.8 g, 162.5 mmol) was carefully dissolved in conc. H₂SO₄ (100 mL). The reaction mixture was cooled to 0° C. and then fuming HNO₃ (6 mL) was added dropwise with stirring. The reaction mixture was allowed to stand for couple of days at 50° C., or until a drop mixed with water, gave no precipitate (the nitro-amine is insoluble in dilute acid). The red reaction mixture was then poured on to crushed ice and bought to pH 5-6 by adding coc. H₄OH solution. The yellow precipitate, which separated, was removed by filtration, washed well with ice water and dried. The solids were impregnated onto silica gel and purified by flash chromatography (silica gel, 40% DCM-EtOAc). 5.2 g of the desired product was obtained (19% yield).

¹H-NMR (400 MHz, DMSO-d₆): δ 8.1 (d, 1H), 7.3 (s.br, 2H), 6.80 (s, 1H). 6.72 (d, 1H). MS (ES) for C₅H₄ClN₃O₂, found 174.0 (MH+).

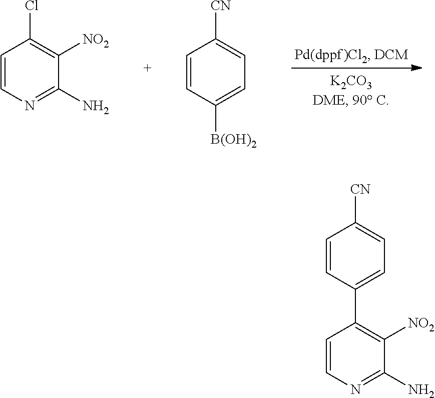

### 4-(2-amino-3-nitropyridin-4-yl)benzonitrile

To a solution of 4-chloro-3-nitropyridin-2-amine (1.95 g, 11.27 mmol) and 4-cyanophenylboronic acid (1.82 g, 12.39 mmol) in DME:H₂O (20 mL:2 mL) was added K₂CO₃ (4.66 g, 33.81 mmol). The reaction mixture was degassed for 0.5 h with N₂. To the reaction mixture was added Pd(dppf)Cl₂.DCM (411 mg, 0.56 mmol, 5 mol %) and was let stir at 95° C. for 2 h. The reaction mixture was diluted with EtOAc and washed with brine and dried over Na₂SO₄. The desired product was obtained on purification by flash chromatography (SiO2, 40% EtOAc-Hexanes). 1.2 g of desired product obtained (yield 45%).

¹H-NMR (400 MHz, DMSO-d₆): δ 8.4 (d, 1H), 8.0 (d, 2H), 7.6 (d, 2H), 6.7 (d, 1H),

MS (ES) for C₁₂H₈N₄O₂, found 239.0 (M−H).

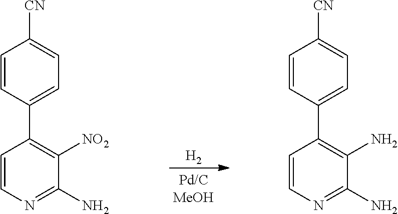

### 4-(2,3-diaminopyridin-4-yl)benzonitrile

To a solution of 4-(2-amino-3-nitropyridin-4-yl)benzonitrile (600 mg, 2.5 mmol) in MeOH was added 10% Pd/C and hydrogenation under balloon pressure. Reaction was complete in 1.5 h. The desired product was obtained on eluting over celite. 514 mg of the desired product obtained (97% yield).

¹H-NMR (400 MHz, DMSO-d₆): δ 7.9 (d, 2H), 7.6 (d, 2H), 7.3 (d, 1H), 6.3 (d, 1H), 5.6 (s, br, 2H), 4.5 (s, br, 2H). MS (ES) for C₁₂H₁₀N₄, found 211.2 (M+H)

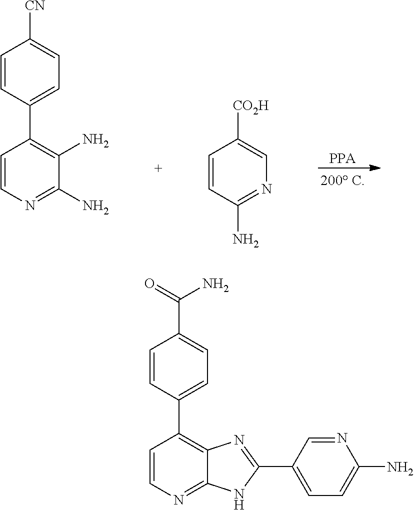

### 4-(2-(6-aminopyridin-3-yl)-3H-imidazo[4,5-b]pyridine-7-yl)benzonitrile

A solution of 4-(2,3-diaminopyridin-4-yl)benzonitrile (105 mg, 0.5 mmol) and 6-aminonicotinic acid (64 mg, 0.5 mmol) in PPA (4 g) was heated at 200° C. for 2 h in a sealed tube. On cooling and addition of water precipitation occurred. The solids filtered. The solids were insoluble material in most of the organic solvents. Dissolved the solids in small amount of DMSO and acetonitrile and purified by Preparative HPLC (reverse phase). Obtained 5 mg of the desired product.

¹H-NMR (400 MHz, DMSO-d₆): δ 8.8 (s, 1H), 8.4 (br, 2H), 8.3 (d, 1H), 8.2 (d, 1H), 8.3 (m, 3H), 7.5 (m, 2H), 6.6 (m, 3H). MS (ES) for C₁₈H₁₄N₆O, found 331.2 (M+H).

### Example 84

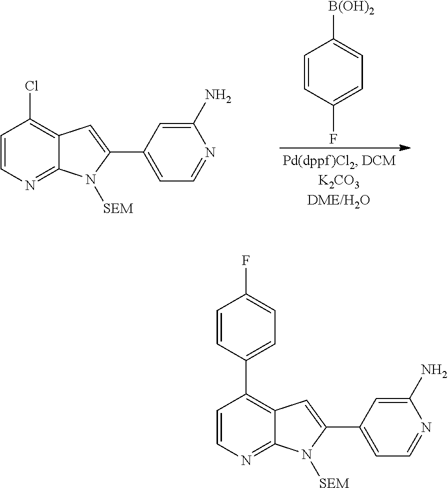

### 4-(4-(4-fluorophenyl)-1-((2-trimethylsilypethoxy)methyl)-1Hpyrrolo[2,3-b]pyridine-2-yl)pyridine-2-amine

To a solution of 4-(4-chloro-1-((2-(trimethylsilyl)ethoxy)methyl)-1H-pyrrolo[2,3-b]pyridin-2-yl)pyrimidin-2-amine (0.100 g, 0.26 mmol, 1 equivalent), 4-fluorophenylboronic acid (0.149 g, 1.06 mmol, 4 equivalent), potassium carbonate (0.287 g, 2.08 mmole, 8 equivalent) in DME:water (4:1, 5 mL) was added diphenylphosphinoferrocene dichloride (0.0.042 g, 0.052 mmol, 20 mol %) The reaction mixture in the pressure tube was sealed under nitrogen and heated at 95° C. overnight.

The reaction was diluted into a large volume of ethyl acetate, washed twice with brine, and dried over sodium sulfate. Product solution was filtered, concentrated and subjected to prep HPLC purification (reverse phase). Obtained 75 mg of the desired product (67% yield)

¹H-NMR (400 MHz, DMSO-d₆): δ 8.4 (d, 1H), 8.2 (m, 1H), 7.8 (m, 1H), 7.2 (m, 4H), 7.1 (d, 1H), 7.0 (s, 1H), 6.9 (s, 1H), 5.8 (s, 2H), 4.8 (s, br, 2H), 3.8 (m, 2H), 1.0 (m, 2H), −0.001 (s, 9H).

MS (ES) for C₂₄H₂₇FN₄OSi, found 435.1 (M+H).

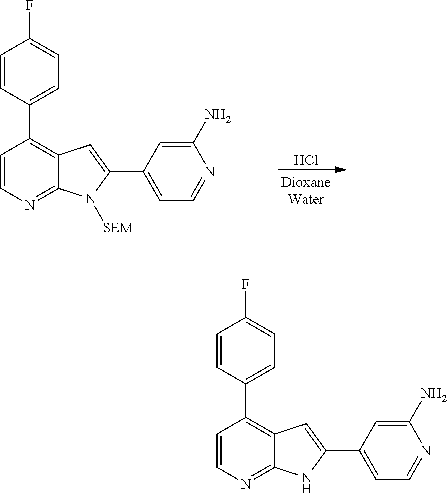

### 4-(4-(4-fluorophenyl)-1H-pyrrolo[2,3-b]pyridine-2-yl)pyridine-2-amine

To a mixture of 4-(4-(4-fluorophenyl)-1-((2-trimethylsilyl)ethoxy)methyl)-1Hpyrrolo[2,3-b]pyridine-2-yl)pyridine-2-amine (75 mg, 0.17 mmol, 1 equivalent) in 6 mL of 4M aqueous HCl was added 2M HCl in dioxane (2 mL). The reaction mixture was stirred overnight at room temperature. The precipitated reaction mixture was concentrated to remove excess HCl and water. The residue was purified by preparative HPLC (reverse phase) to give 25 mg of the pure title compound (50% yield).

¹H-NMR (400 MHz, DMSO-d₆): δ 12.40 (s, 1H), 8.3 (d, 1H), 8.0 (d, 1H), 7.9 (d, 2H), 7.4 (m, 2H), 7.3 (s, 1H), 7.2 (m, 2H), 6.9 (s, 1H), 6.0 (s, 2H). MS (ES) for C₁₈H₁₃FN₄, found 305.1 (MH+).

### Example 85

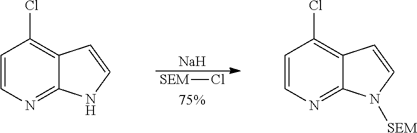

### 4-Chloro-1-((2-(trimethylsilyl)ethoxy)methyl)-1H-pyrrolo[2,3-b]pyridine

4-Chloroazaindole (8.3206 g, 54.53 mmol, 1 equivalent) was weighed out and added to a dry flask with magnetic stir bar. This was dissolved in dimethylformamide (136 mL), put under an atmosphere of nitrogen, and cooled to −45° C. in a bath of dry ice/acetonitrile. Sodium hydride (95 wt %, 1.5292 g, 60.53 mmol, 1.11 equivalent) was weighed out and added carefully. When the bubbling began to slow, the cooling bath was removed and the reaction was stirred for 15 minutes warming to room temperature. It was cooled back to −45° and 2-(trimethylsilyl)ethoxymethyl chloride (10.6 mL, 59.89 mmol, 1.10 equivalent) was added. This was allowed to stir overnight, warming to room temperature.

The reaction was poured into 500 mL ethyl acetate and washed twice with 10% aqueous lithium chloride. It was then washed successively with brine and water and dried over sodium sulfate. Product solution was filtered, concentrated onto a plug of silica and chromatographed in 5% ethyl acetate in hexane going up to 10%. Pure product was combined, concentrated, and dried on the high vacuum to give 11.498 g of product (75% yield).

¹H-NMR (400 MHz, DMSO-d₆): δ 8.37 (d, 1H), 7.90 (d, 1H), 7.40 (d, 1H), 6.72 (d, 1H), 5.76 (s, 2H), 3.62 (t, 2H), 0.93 (t, 2H), 0.01 (s, 9H). MS (ES) for C₁₃H₁₉ClN₂OSi, found 283.1 (MH+).

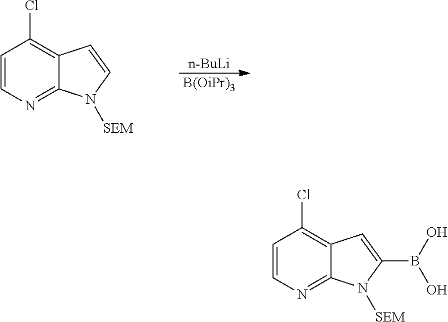

### 4-Chloro-1-((2-(trimethylsilypethoxy)methyl)-1H-pyrrolo[2,3-b]pyridin-2-ylboronic acid

4-Chloro-1-((2-(trimethylsilyl)ethoxy)methyl)-1H-pyrrolo[2,3-b]pyridine was concentrated in the tared reaction flask and weighed (10.4722 g, 37.02 mmol, 1 equivalent). This was azeotroped twice with toluene, and dried under high vacuum. The reaction flask was back-filled with dry nitrogen, evacuated, and filled with nitrogen again. The substrate was dissolved in 95 mL of tetrahydrofuran, and the solution was cooled to −45° C. with dry ice/acetonitrile. 28 mL of n-butyllithium solution (1.6 M in hexane, 44.8 mmol, 1.2 equivalents) was added slowly, and the reaction was stirred at −45° for 1 hour. Triisopropyl borate (11.1 mL, 48.1 mmol, 1.3 equivalents) was added, and the reaction was stirred overnight warming to room temperature.

The reaction was quenched with 1 M aqueous HCl and stirred 20 minutes before neutralizing with saturated aqueous potassium phosphate (dibasic). This was extracted four time with diethyl ether, and the combined organic layer was dried over sodium sulfate, filtered, concentrated, and dried on the high vacuum to give 11.808 g (98% recovery). This was kept stored in the freezer, and used as-is without further purification.

¹H-NMR (400 MHz, DMSO-d₆): δ 8.75 (s, 2H), 8.42 (d, 1H), 7.41 (d, 1H), 7.32 (s, 1H), 6.05 (s, 2H), 3.58 (t, 2H), 0.90 (t, 2H), −0.001 (s, 9H). MS (ES) for C₁₃H₂₀BClN₂O₃Si, found 327.1 (MH+).

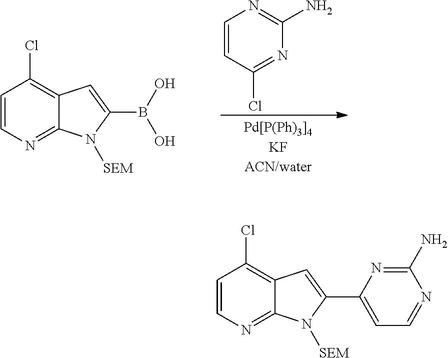

### 4-(4-chloro-1-((2-(trimethylsilypethoxy)methyl)-1H-pyrrolo[2,3-b]pyridin-2-yl)pyrimidin-2-amine

4-Chloro-1-((2-(trimethylsilyl)ethoxy)methyl)-1H-pyrrolo[2,3-b]pyridin-2-ylboronic acid (0.6807 g, 2.084 mmol, 1 equivalent) was weighed out and added to a reaction tube with magnetic stir bar. 4-chloropyrimidin-2-amine (0.2419 g, 1.87 mmol, 0.90 equivalent) was weighed out and added, along with potassium fluoride (0.3627 g, 6.24 mmol, 3 equivalents). 5.0 mL of acetonitrile and 2.5 mL of water were added. The solution was subjected to vigorous subsurface nitrogen sparge, and then tetrakis triphenylphosphine palladium (0.0722 g, 0.0625 mmol, 3 mol %) was weighed out and added. The tube was sealed under nitrogen and heated at 95° C. for 4 hours. The reaction was diluted into a large volume of ethyl acetate, washed twice with brine, and dried over sodium sulfate. Product solution was filtered, concentrated onto a silica plug and chromatographed in 98/2 dichloromethane/methanol. This gave a clean mixture of the desired product and residual 4-chloropyrimidin-2-amine. This was purified on reverse phase by preparative HPLC to give 0.4835 g of the pure title compound (56% yield).

¹H-NMR (400 MHz, DMSO-d₆): δ 8.58 (d, 2H), 7.61 (d, 1H), 7.52 (s, 1H), 7.44 (d, 1H), 7.08 (s, 2H), 6.56 (s, 2H), 3.58 (t, 2H), 0.93 (t, 2H), −0.001 (s, 9H).

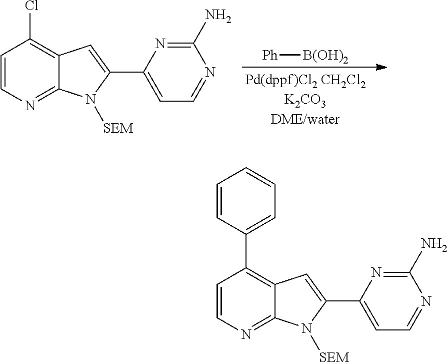

### 4-(4-phenyl-1-((2-(trimethylsilypethoxy)methyl)-1H-pyrrolo[2,3-b]pyridin-2-yl)pyrimidin-2-amine

4-(4-chloro-14(2-(trimethylsilyl)ethoxy)methyl)-1H-pyrrolo[2,3-b]pyridin-2-yl)pyrimidin-2-amine was concentrated in a tared flask, dried on the high vacuum and weighed (0.1085 g, 0.29 mmol, 1 equivalent). It was transferred into a reaction tube with magnetic stir bar in a total of 4.0 mL dimethoxyethane. 0.4 mL water was added and phenyl boronic acid (0.1414 g, 1.16 mmol, 4 equivalents) was weighed out and added, followed by potassium carbonate (0.3197 g, 2.31 mmol, 8 equivalents). The solution was subjected to vigorous subsurface nitrogen sparge, and then palladium diphenylphosphinoferrocene dichloride (mono dichloromethane adduct) was weighed out and added (0.0427 g, 0.052 mmol, 18 mol %). The tube was sealed under nitrogen and heated at 95° C. overnight.

The reaction was diluted into a large volume of ethyl acetate, washed twice with brine, and dried over sodium sulfate. Product solution was filtered, concentrated onto a silica plug and chromatographed in 50/50 hexane/ethyl acetate. After drying on the high vacuum, 0.098 g of pure product was obtained (81% yield).

¹H-NMR (400 MHz, DMSO-d₆): 8.66 (d, 1H), 8.52 (d, 1H), 8.04 (d, 2H), 7.81 (t, 2H), 7.73 (t, 1H), 7.57 (d, 2H), 7.41 (d, 1H), 7.01 (s, 2H), 6.57 (s, 2H), 3.61 (t, 2H), 0.93 (t, 2H), −0.001 (s, 9H).

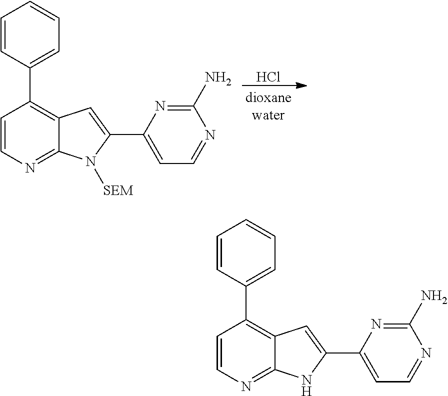

### 4-(4-phenyl-1H-pyrrolo[2,3-b]pyridin-2-yl)pyrimidin-2-amine

4-(4-phenyl-14(2-(trimethylsilyl)ethoxy)methyl)-1H-pyrrolo[2,3-b]pyridin-2-yl)pyrimidin-2-amine was concentrated in the tared reaction flask and weighed (0.091 g, 0.218 mmol, 1 equivalent). A magnetic stir bar was added along with 18 mL 4M aqueous HCl and 18 mL 4M HCl in dioxane solution. This was heated at 100° C. for 3.5 hours. After that time, it was quenched with sodium hydrogen carbonate and extracted 4 times with ethyl acetate. The ethyl acetate layer was washed twice with brine and dried over sodium sulfate. The product solution was filtered and concentrated onto a plug of silica. This was chromatographed in 2% to 5% (7M ammonia solution in methanol) in dichloromethane. After drying on the high vacuum, 0.0470 g of pure product was obtained (75% yield).

¹H-NMR (400 MHz, DMSO-d₆): δ 12.42 (s, 1H), 8.39 (d, 1H), 8.34 (d, 1H), 7.81 (d, 2H), 7.60 (t, 2H), 7.52 (t, 1H), 7.38 (s, 1H), 7.28 (d, 1H), 7.26 (d, 1H), 6.65 (s, 2H). MS (ES) for C₁₇H₁₃N₆, found 288.3 (MH+).

### Example 86

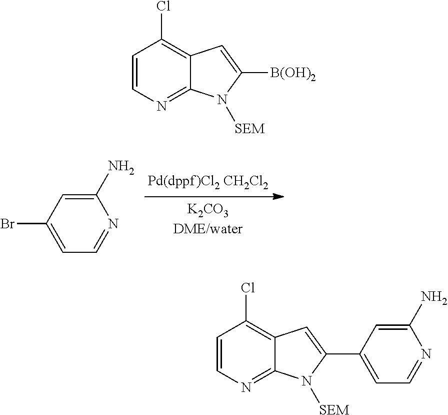

### 4-(4-chloro-1-((2-(trimethylsilypethoxy)methyl)-1H-pyrrolo[2,3-b]pyridin-2-yl)pyridin-2-amine

4-Chloro-1-((2-(trimethylsilyl)ethoxy)methyl)-1H-pyrrolo[2,3-b]pyridin-2-ylboronic acid (2.0282 g, 6.21 mmol, 1 equivalent) was weighed out and added to a reaction tube with magnetic stir bar. 4-bromopyridin-2-amine (1.0737 g, 6.21 mmol, 1 equivalent) was weighed out and added, followed by potassium carbonate (2.5749 g, 18.63 mmol, 3 equivalents). 88 mL of dimethoxyethane and 8.8 mL of water were added. The solution was subjected to vigorous subsurface nitrogen sparge, and then palladium diphenylphosphinoferrocene dichloride (mono dichloromethane adduct) was weighed out and added (0.5071 g, 0.621 mmol, 10 mol %). The tube was sealed under nitrogen and heated at 100° C. overnight.

The reaction was diluted into a large volume of ethyl acetate, washed twice with brine, and dried over sodium sulfate. Product solution was filtered, concentrated onto a silica plug and chromatographed in hexane/ethyl acetate. After high vacuum, this gave 1.521 g of a clean mixture of desired product and residual 4-bromopyridin-2-amine. This was purified on reverse phase by preparative HPLC to give 0.949 g of the pure title compound (41% yield).

¹H-NMR (400 MHz, DMSO-d₆): δ 8.44 (d, 1H), 8.15 (d, 1H), 7.49 (d, 1H), 7.04 (dd, 1H), 6.94 (s, 1H), 6.91 (d, 1H), 6.25 (s, 2H), 5.81 (s, 2H), 3.68 (t, 2H), 0.96 (t, 2H), −0.001 (s, 9H). MS (ES) for C₁₈H₂₃ClN₄OSi, found 375.3 (MH+).

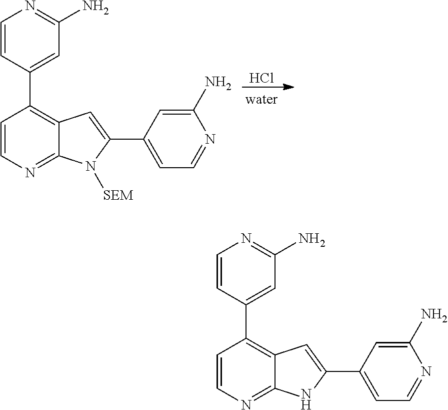

### 4,4′-(1H-pyrrolo[2,3-b]pyridine-2,4-diyl)dipyridin-2-amine

4,4′-(14(2-(trimethylsilyl)ethoxy)methyl)-1H-pyrrolo[2,3-b]pyridine-2,4-diyl)dipyridin-2-amine was concentrated in the reaction flask and dried on the high vacuum (0.106 g, 0.245 mmol, 1 equivalent). A magnetic stir bar was added, followed by 13.0 mL of 6M aqueous hydrochloric acid. This was stirred overnight at room temperature.

The precipitated reaction mixture was concentrated to remove excess HCl and water. The residue was purified on reverse phase by preparative HPLC to give 46.0 mg of the pure title compound after drying (62% yield).

¹H-NMR (400 MHz, DMSO-d₆): δ 12.48 (s, 1H), 8.35 (d, 1H), 8.07 (d, 1H), 7.99 (d, 1H), 7.21 (d, 1H), 7.11 (s, 1H), 7.08 (d, 1H), 6.96 (s, 1H), 6.89 (s, 1H), 6.88 (d, 1H), 6.13 (s, 2H), 6.00 (s, 2H). MS (ES) for C₁₇H₁₄N₆, found 303.3 (MH+).

Using analogous synthetic techniques as in Example 86, and substituting with the appropriate alternative starting materials, the following compounds were prepared. Alternative starting materials were obtained commercially unless otherwise indicated.

### {3-[2-(2-aminopyrimidin-4-yl)-1H-pyrrolo[2,3-b]pyridin-4-yl]phenyl}methanol

¹H-NMR (400 MHz, DMSO-d₆): δ 12.39 (s, 1H), 8.38 (d, 1H), 8.34 (d, 1H), 7.74 (s, 1H), 7.67 (d, 1H), 7.55 (t, 1H), 7.45 (d, 1H), 7.36 (s, 1H), 7.27 (d, 1H), 7.22 (d, 1H), 6.64, (s, 2H), 5.37 (s, 1H), 4.63 (s, 2H). MS (ES) for C₁₈H₁₅N₅O, found 318.3 (MH+).

### 4(4-pyridin-4-yl-1H-pyrrolo[2,3-b]pyridin-2-yl)pyrimidin-2-amine

¹H-NMR (400 MHz, DMSO-d₆): δ 12.55 (s, 1H), 8.79 (d, 2H), 8.45 (d, 1H), 8.36 (d, 1H), 7.82 (d, 2H), 7.42 (s, 1H), 7.36, (d, 1H), 7.30 (d, 1H), 6.66 (s, 2H). MS (ES) for C₁₆H₁₂N₆, found 289.1 (MH+).

### 3-[2-(2-aminopyrimidin-4-yl)-1H-pyrrolo[2,3-b]pyridin-4-yl]-N,N-dimethylbenzamide

¹H-NMR (400 MHz, DMSO-d₆): δ 12.47 (s, 1H), 8.41 (d, 1H), 8.35 (d, 1H), 7.88 (dt, 1H), 7.77 (t, 1H), 7.67 (t, 1H), 7.54 (dt, 1H), 7.34 (s, 1H), 7.28 (dd, 2H), 6.67 (s, 2H), 3.03 (s, 3H), 2.99 (s, 3H). MS (ES) for C₂₀H₁₈N₆O, found 359.3 (MH+).

### 4-{4-[3-(chloromethyl)phenyl]-1H-pyrrolo[2,3-b]pyridin-2-yl}pyrimidin-2-amine

¹H-NMR (400 MHz, CDCl₃): δ 8.39 (d, 1H), 8.29 (d, 1H), 7.78 (s, 1H), 7.72 (d, 1H), 7.57 (t, 1H), 7.52 (d, 1H), 7.26 (s, 1H), 7.21 (d, 1H), 7.09 (d, 1H), 4.72 (s, 2H). MS (ES) for C₁₈H₁₄ClN₅, found 336.2 (MH+).

### 4-(4-phenyl-1H-pyrrolo[2,3-b]pyridin-2-yl)pyridin-2-amine

¹H-NMR (400 MHz, DMSO-d₆): δ 12.39 (s, 1H), 8.32 (d, 1H), 7.96 (d, 1H), 7.80 (d, 2H), 7.57 (t, 2H), 7.48 (t, 1H), 7.21 (d, 1H), 7.08 (s, 1H), 7.07 (d, 1H), 6.94 (s, 1H), 5.95 (s, 2H). MS (ES) for C₁₈H₁₄N₄, found 287.2 (MH+).

### 4-[4-(2-aminopyridin-4-yl)-1H-pyrrolo[2,3-b]pyridin-2-yl]pyrimidin-2-amine

¹H-NMR (400 MHz, DMSO-d₆): δ 8.59 (d, 1H), 8.51 (d, 1H), 8.34 (br s, 2H), 8.16 (d, 1H), 7.67 (s, 1H), 7.59 (d, 1H), 7.48 (s, 1H), 7.44 (d, 1H), 7.26 (d, 1H). MS (ES) for C₁₆H₁₃N₇, found 304.3 (MH+).

### Example 87

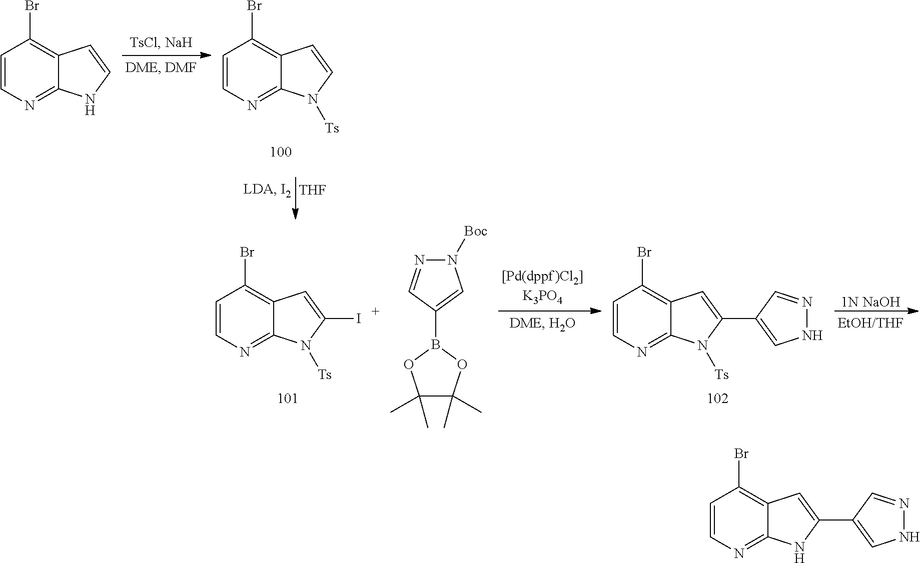

### 4-Bromo-1-tosyl-1H-pyrrolo[2,3-b]pyridine (100)

The title compound was synthesized in a manner similar to Example 76, with 4-bromo-1 H-pyrrolo[2,3-b]pyridine instead of 4-iodo-1H-pyrrolo[2,3-b]pyridine as substrate.

¹H-NMR (400 MHz, CDCl₃): δ 8.22 (d, 1H), 8.06 (d, 2H), 7.78 (d, 1H), 7.35 (d, 1H), 7.26 (m, 2H), 6.63 (s, 1H), 2.37 (s, 3H). MS (ES) for C₁₄H₁₁BrN₂O₂S, found 351, 353 (MH+).

### 4-Bromo-2-iodo-1-tosyl-1H-pyrrolo[2,3-b]pyridine (101)

The title compound was made following the known procedure as referenced in WO2008/034860.

¹H-NMR (400 MHz, CDCl₃): δ 8.17 (m, 1H), 8.07 (d, 1H), 7.29 (m, 4H), 7.03 (s, 1H), 2.34 (s, 3H). ¹³C-NMR (100 MHz, CDCl₃): δ 149.3, 145.9, 144.9, 135.6, 130.0, 128.5, 125.5, 123.9, 122.6, 119.6, 21.9. MS (ES) for C₁₄H₁₀BrIN₂O₂S, found 477, 479 (MH+).

### 4-Bromo-2-(1H-pyrazol-4-yl)-1-tosyl-1H-pyrrolo[2,3-b]pyridine (102)

A pressure tube was charged with 4-bromo-2-iodo-1-tosyl-1H-pyrrolo[2,3-b]pyridine (101, 62 mg, 0.129 mmol), tert-butyl 4-(4,4,5,5-tetramethyl-1,3,2-dioxaborolan-2-yl)-1H-pyrazole-1-carboxylate (80 mg, 0.272 mmol), 1,1′-bis(diphenylphosphino)ferrocene-palladium(II) dichloride dichloromethane adduct (9.4 mg, 0.012 mmol), potassium phosphate (60 mg, 0.283 mmol), DME (2 mL), and water (0.4 mL). After purging the mixture with nitrogen gas, the tube was sealed and heated at 90° C. for 18 h. The mixture was cooled to room temperature, concentrated to dryness, taken up with toluene/dichloromethane and directly loaded onto a silica gel column. The title compound was obtained after purification by flash chromatography (7:3 hexanes/ethyl acetate) as a solid (8 mg, 15% yield).

¹H-NMR (400 MHz, CDCl₃): δ 8.28 (d, 1H), 7.90 (br s, 2H), 7.69 (d, 2H), 7.38 (d, 2H), 7.17 (d, 2H), 6.56 (s, 1H), 2.34 (s, 3H). MS (ES) for C₁₇H₁₃BrN₄O₂S, found 263, 265 (MH+).

### 4-Bromo-2-(1H-pyrazol-4-yl)-1H-pyrrolo[2,3-b]pyridine

The title compound was synthesized in a manner similar to Example 77, with 4-bromo-2-(1H-pyrazol-4-yl)-1-tosyl-1H-pyrrolo[2,3-b]pyridine (102) instead of 4-(2-(methylthio)pyrimidin-4-yl)-1-tosyl-1H-pyrrolo[2,3-b]pyridine (99) as substrate.

¹H-NMR (400 MHz, CD₃OD): δ 8.11 (m, 2H), 7.94 (d, 1H), 7.26 (d, 1H), 6.63 (s, 1H). MS (ES) for C₁₀H₇BrN₄, found 263, 265 (MH+).

### Example 88

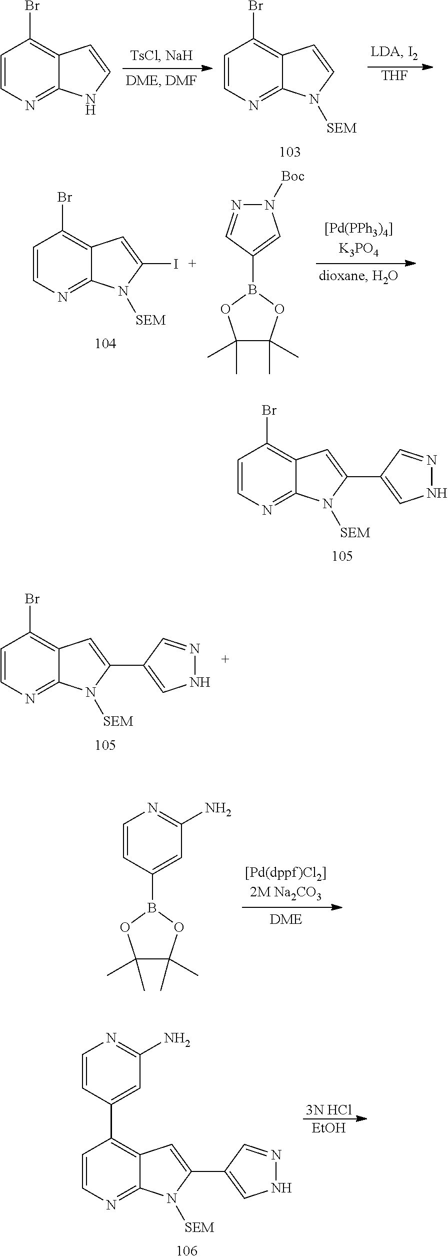

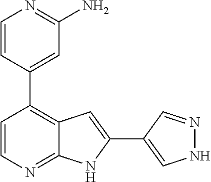

### 4-Bromo-1-((2-(trimethylsilyl)ethoxy)methyl)-1H-pyrrolo[2,3-b]pyridine (103)

The title compound was synthesized in a manner similar to Example 87, with SEMCI instead of TsCl as reagent.

¹H-NMR (400 MHz, CDCl₃): δ 8.19 (d, 1H), 7.45 (m, 1H), 7.33 (d, 1H), 6.62 (s, 1H), 5.72 (s, 2H), 3.60 (m, 2H), 0.97 (m, 2H), 0.01 (s, 9H). ¹³C-NMR (100 MHz, CDCl₃): δ 149.4, 144.8, 129.8, 126.6, 123.5, 121.0, 102.6, 74.6, 87.7, 19.1, 0.00. MS (ES) for C₁₃H₁₉BrN₂OSi, found 327, 329 (MH+).

### 4-Bromo-2-iodo-14(2-(trimethylsilyl)ethoxy)methyl)-1H-pyrrolo[2,3-b]pyridine (104)

The title compound was synthesized in a manner similar to Example 35, with 4-bromo-1-((2-(trimethylsilyl)ethoxy)methyl)-1H-pyrrolo[2,3-b]pyridine (103) instead of 4-bromo-1-tosyl-1H-pyrrolo[2,3-b]pyridine (100) as substrate.

¹H-NMR (400 MHz, CDCl₃): δ 8.11 (d, 2H), 7.30 (m, 1H), 6.96 (s, 1H), 5.76 (s, 2H), 3.64 (m, 2H), 0.98 (m, 2H), 0.00 (s, 9H). MS (ES) for C₁₃H₁₈BrIN₂OSi, found 453, 455 (MH+). 2

### 4-Bromo-2-(1H-pyrazol-4-yl)-14(2-(trimethylsilyl)ethoxy)methyl)-1H-pyrrolo[2,3-b]pyridine (105)

The title compound was synthesized in a manner similar to Example 45, with 4-bromo-2-iodo-1-((2-(trimethylsilyl)ethoxy)methyl)-1H-pyrrolo[2,3-b]pyridine (104) instead of 4-bromo-2-iodo-1-tosyl-1H-pyrrolo[2,3-b]pyridine (101) as substrate, tetrakis(triphenylphosphine)palladium(0) instead of 1,1′-bis(diphenylphosphino)ferrocene-palladium(II) dichloride dichloromethane adduct as catalyst, and dioxane instead of DME as solvent.

¹H-NMR (400 MHz, CDCl₃): δ 11.2 (br, 1H), 8.22 (d, 2H), 8.14 (d, 1H), 7.36 (m, 1H), 6.71 (s, 1H), 5.79 (s, 2H), 3.77 (m, 2H), 1.04 (m, 2H), 0.00 (s, 9H). MS (ES) for C₁₆H₂₁BrN₄OSi, found 393, 395 (MH+).

### 4-(2-(1H-Pyrazol-4-yl)-1-((2-(trimethylsilyl)ethoxy)methyl)-1H-pyrrolo[2,3-b]pyridin-4-yl)pyridin-2-amine (106)

The title compound was synthesized in a manner similar to Example 45, with 4-bromo-2-(1H-pyrazol-4-yl)-14(2-(trimethylsilyl)ethoxy)methyl)-1H-pyrrolo[2,3-b]pyridine (105) instead of 4-bromo-2-iodo-1-tosyl-1H-pyrrolo[2,3-b]pyridine (101) as substrate, with 4-(4,4,5,5-tetramethyl-1,3,2-dioxaborolan-2-yl)pyridin-2-amine and 2M sodium carbonate instead of tert-butyl 4-(4,4,5,5-tetramethyl-1,3,2-dioxaborolan-2-yl)-1H-pyrazole-1-carboxylate and potassium phosphate as reagents.

¹H-NMR (400 MHz, CDCl₃): δ 8.38 (d, 1H), 8.26 (d, 1H), 8.18 (s, 2H), 7.21 (d, 1H), 7.07 (m, 1H), 6.90 (s, 1H), 6.82 (s, 1H), 5.82 (s, 2H), 3.79 (m, 2H), 1.03 (m, 2H), 0.01 (s, 9H). ¹³C-NMR (100 MHz, CDCl₃): δ 170.2, 160.3, 159.1, 151.5, 149.9, 144.0, 139.8, 136.2, 119.8, 116.9, 115.3, 114.3, 112.1, 109.2, 99.1, 71.7, 67.6, 19.4, 0.00. MS (ES) for C₂₁H₂₆N₆OSi, found 407 (MH+).

### 4-[2-(1H-Pyrazol-4-yl)-1H-pyrrolo[2,3-b]pyridin-4-yl]pyridin-2-amine

The title compound was obtained by treatment of 4-(2-(1H-pyrazol-4-yl)-14(2-(trimethylsilyl)ethoxy)methyl)-1H-pyrrolo[2,3-b]pyridin-4-yl)pyridin-2-amine (106, 56 mg, 0.138 mmol) in abs ethanol (5 mL) with 3 N HCl (1.5 mL) at 85° C. for 18 h. After concentration to dryness, the mixture was dissolved in MeOH and stirred with basic ion exchange Bio-Rad® AG 1-x8 resin (50 mg, hydroxide form) for 10 min, until a basic pH was reached. The surnatant was discarded after filtration through septum, then the recovered resin was treated with EtOH and 6 N HCl. The suspension was filtered through fritted septum. The clear solution was concentrated and purified by preparative HPLC (reverse-phase, 0.1% TFA in acetonitrile/0.05% TFA in water). After removal of volatiles under reduced pressure and freeze-drying, the title compound was obtained as a solid (8 mg, 21% yield).

¹H-NMR (400 MHz, CDCl₃/CD₃OD): δ 8.30 (m, 1H), 8.13 (br s, 1H), 7.96 (d, 1H), 7.43 (m, 1H), 7.30 (m, 1H), 6.86 (s, 1H). MS (ES) for C₁₅H₁₂N₆, found 277 (MH+).

### Example 89

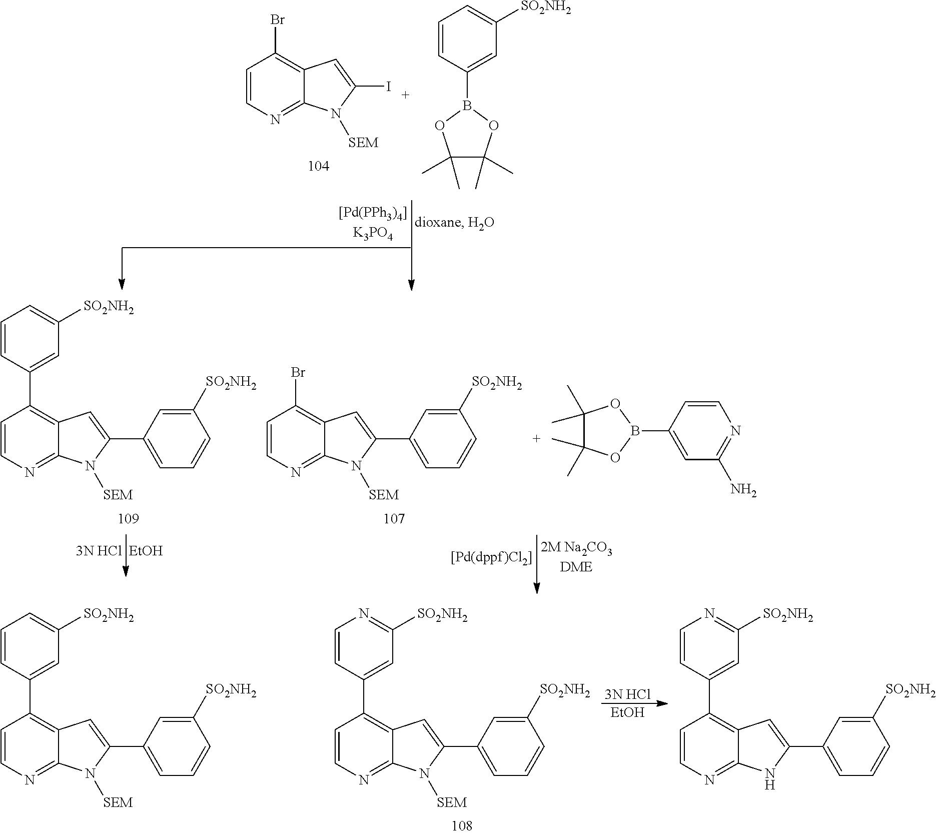

### 3-(4-bromo-1-((2-(trimethylsilyl)ethoxy)methyl)-1H-pyrrolo[2,3-b]pyridin-2-yl)benzenesulfonamide (107)

The title compound was synthesized in a manner similar to Example 88, with 3-(4,4,5,5-tetramethyl-1,3,2-dioxaborolan-2-yl)benzenesulfonamide instead of tert-butyl 4-(4,4,5,5-tetramethyl-1,3,2-dioxaborolan-2-yl)-1H-pyrazole-1-carboxylate as reagent.

¹H-NMR (400 MHz, CDCl₃): δ 8.39 (m, 1H), 8.18 (d, 1H), 8.08 (m, 1H), 8.00 (m, 1H), 7.65 (t, 1H), 7.36 (d, 1H), 6.73 (s, 1H), 5.65 (s, 2H), 3.76 (m, 2H), 1.03 (m, 2H), 0.00 (s, 9H). MS (ES) for C₁₉H₂₄BrN₃O₃SSi, found 482, 484 (MH+).

3-(4-(2-Aminopyridin-4-yl)-1-((2-(trimethylsilyl)ethoxy)methyl)-1H-pyrrolo[2,3-b]pyridin-2-yl)benzenesulfonamide (108).

The title compound was synthesized in a manner similar to Example 88, with 3-(4-bromo-1-((2-(trimethylsilyl)ethoxy)methyl)-1H-pyrrolo[2,3-b]pyridin-2-yl)benzenesulfonamide (107) instead of 4-bromo-2-(1H-pyrazol-4-yl)-1-((2-(trimethylsilyl)ethoxy)methyl)-1H-pyrrolo[2,3-b]pyridine (105) as substrate.

¹H-NMR (400 MHz, CDCl₃): δ 8.36 (d, 1H), 8.32 (s, 1H), 8.05 (m, 2H), 7.95 (d, 1H), 7.55 (t, 1H), 7.10 (d, 1H), 6.81 (d, 1H), 6.76 (d, 2H), 5.65 (s, 2H), 3.80 (m, 2H), 1.03 (m, 2H), 0.00 (s, 9H). MS (ES) for C₂₄H₂₉N₅O₃SSi, found 496 (MH+).

### 3-[4-(2-Aminopyridin-4-yl)-1H-pyrrolo[2,3-b]pyridin-2-yl]benzenesulfonamide

The title compound was synthesized in a manner similar to Example 88, with 3-(4-(2-aminopyridin-4-yl)-1-((2-(trimethylsilyl)ethoxy)methyl)-1H-pyrrolo[2,3-b]pyridin-2-yl)benzenesulfonamide (108) instead of 4-(2-(1H-pyrazol-4-yl)-14(2-(trimethylsilyl)ethoxy)methyl)-1H-pyrrolo[2,3-b]pyridin-4-yl)pyridin-2-amine (106) as substrate, and without treatment with basic ion exchange Bio-Rad® AG 1-x8 resin.

¹H-NMR (400 MHz, CDCl₃/CD₃OD): δ 8.42 (m, 1H), 8.10 (m, 1H), 7.97 (d, 1H), 7.94 (m, 1H), 7.76 (s, 1H), 7.68 (t, 1H), 7.44 (m, 1H), 7.35 (d, 1H), 7.31 (dd, 1H), 7.18 (s, 1H). MS (ES) for C₁₈H₁₅N₅O₂S found 366 (MH+).

### 3,3═-(1((2-(Trimethylsilyl)ethoxy)methyl)-1H-pyrrolo[2,3-b]pyridine-2,4-diyl)dibenzenesulfonamide (109)

The title compound was obtained as side-product from the preparation of compound 107 (14% yield), as described above.

¹H-NMR (400 MHz, CDCl₃/CD₃OD): δ 8.45 (d, 1H), 8.33 (m, 1H), 7.98-8.10 (m, 4H), 7.66-7.75 (m, 2H), 7.33 (d, 1H), 6.93 (s, 1H), 3.79 (m, 2H), 1.03 (m, 2H), 0.00 (s, 9H). MS (ES) for C₂₅H₃₀N₄O₅S₂Si, found 559 (MH+).

### 3,3′-(1H-Pyrrolo[2,3-b]pyridine-2,4-diyl)dibenzenesulfonamide

The title compound was synthesized in a manner similar to Example 88, with 3,3′-(1-((2-(trimethylsilyl)ethoxy)methyl)-1H-pyrrolo[2,3-b]pyridine-2,4-diyl)dibenzenesulfonamide (109) instead of 3-(4-(2-aminopyridin-4-yl)-1-((2-(trimethylsilyl)ethoxy)methyl)-1H-pyrrolo[2,3-b]pyridin-2-yl)benzenesulfonamide (108) as substrate, and without treatment with basic ion exchange Bio-Rad® AG 1-x8 resin.

¹H-NMR (400 MHz, d₆-dmso): δ 8.42 (m, 1H), 8.39 (d, 1H), 8.23 (m, 1H), 8.19 (m, 1H), 8.07 (m, 1H), 7.94 (m, 1H), 7.81 (m, 2H), 7.70 (t, 1H), 7.53 (s, 2H), 7.44 (s, 2H), 7.27 (d, 1H), 7.19 (m, 1H), 7.09 (t, 1H). MS (ES) for C₁₉H₁₆N₄O₄S₂ found 429 (MH+).

### Example 90

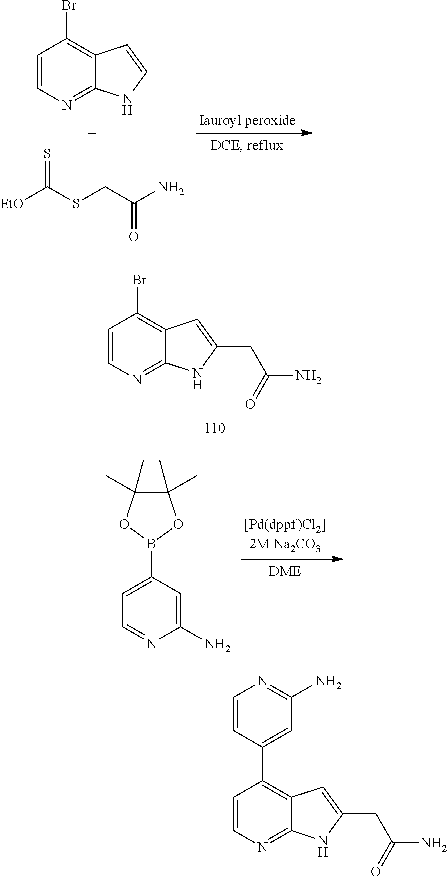

### 2-(4-Bromo-1H-pyrrolo[2,3-b]pyridin-2-yl)acetamide (110)

The title compound was made following the known procedure as referenced in WO2008/034860.

¹H-NMR (400 MHz, CD₃OD): δ 7.93 (d, 1H), 7.21 (d, 1H), 6.37 (s, 1H), 3.30 (s, 2H).

### 2-[4-(2-Aminopyridin-4-yl)-1H-pyrrolo[2,3-b]pyridin-2-yl]acetamide

The title compound was synthesized in a manner similar to Example 88, with 2-(4-bromo-1 H-pyrrolo[2,3-b]pyridin-2-yl)acetamide (110) instead of 4-bromo-2-(1H-pyrazol-4-yl)-1-((2-(trimethylsilyl)ethoxy)methyl)-1H-pyrrolo[2,3-b]pyridine (105) as substrate.

¹H-NMR (400 MHz, CD₃OD): δ 8.22 (d, 1H), 8.01 (m, 1H), 7.22 (m, 1H), 7.01 (m, 2H), 6.57 (s, 1H), 3.78 (s, 2H). MS (ES) for C₁₄H₁₃N₅O, found 268 (MH+).

### Example 91

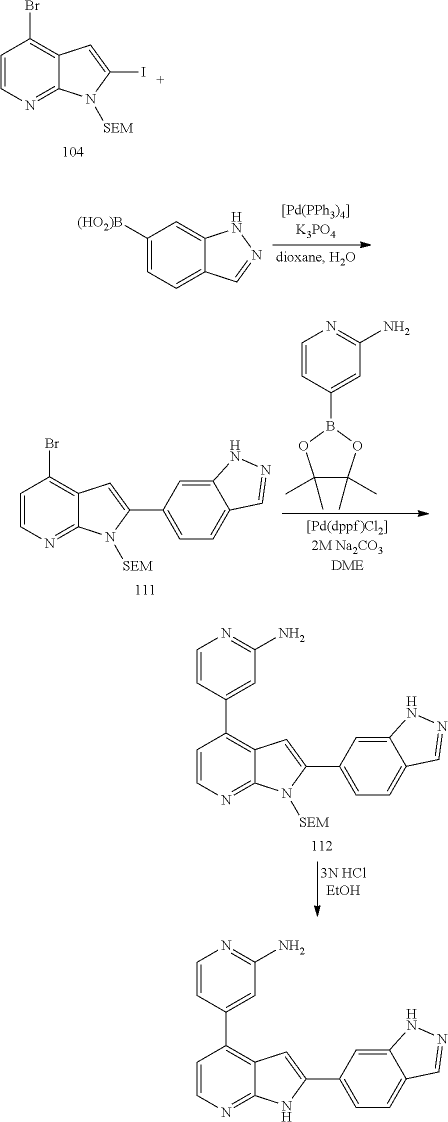

### 6-(4-Bromo-1-((2-(trimethylsilyl)ethoxy)methyl)-1H-pyrrolo[2,3-b]pyridin-2-yl)-1H-indazole (111)

The title compound was synthesized in a manner similar to Example 87, with 4-bromo-2-iodo-14(2-(trimethylsilyl)ethoxy)methyl)-1H-pyrrolo[2,3-b]pyridine (104) instead of 4-bromo-2-iodo-1-tosyl-1H-pyrrolo[2,3-b]pyridine (101) as substrate, 1H-indazol-6-ylboronic acid instead of 4-(4,4,5,5-tetramethyl-1,3,2-dioxaborolan-2-yl)pyridin-2-amine as reagent, tetrakis(triphenylphosphine)palladium(0) instead of 1,1′-bis(diphenylphosphino)ferrocene-palladium(II) dichloride dichloromethane adduct as catalyst, and dioxane instead of DME as solvent.

¹H-NMR (400 MHz, CDCl₃): δ 12.2 (br, 1H), 8.20 (m, 2H), 8.10 (m, 1H), 7.91 (d, 1H), 7.62 (dd, 1H), 7.36 (d, 1H), 6.76 (s, 1H), 5.77 (s, 2H), 3.84 (m, 2H), 1.02 (m, 2H), 0.00 (s, 9H). MS (ES) for C₂₀H₂₃BrN₄OSi, found 443, 445 (MH+).

### 4-(2-(1H-Indazol-6-yl)-1-((2-(trimethylsilyl)ethoxy)methyl)-1H-pyrrolo[2,3-b]pyridin-4-yl)pyridin-2-amine (112)

The title compound was synthesized in a manner similar to Example 87 6-(4-bromo-1-((2-(trimethylsilyl)ethoxy)methyl)-1H-pyrrolo[2,3-b]pyridin-2-yl)-1H-indazole (111) instead of 4-bromo-2-iodo-1-tosyl-1H-pyrrolo[2,3-b]pyridine (101) as substrate, with 4-(4,4,5,5-tetramethyl-1,3,2-dioxaborolan-2-yl)pyridin-2-amine and 2M sodium carbonate instead of tert-butyl 4-(4,4,5,5-tetramethyl-1,3,2-dioxaborolan-2-yl)-1H-pyrazole-1-carboxylate and potassium phosphate as reagents.

¹H-NMR (400 MHz, CDCl₃): δ 10.2 (br, 1H), 8.31 (d, 1H), 8.16 (m, 1H), 8.09 (m, 2H), 7.84 (d, 1H), 7.75 (dd, 1H), 7.20 (m, 2H), 6.93 (m, 2H), 6.11 (m, 1H), 5.77 (s, 2H), 3.85 (m, 2H), 1.03 (m, 2H), 0.00 (s, 9H). MS (ES) for C₂₅H₂₈N₆OSi, found 457 (MH+).

### 4-[2-(1H-Indazol-6-yl)-1H-pyrrolo[2,3-b]pyridin-4-yl]pyridin-2-amine

The title compound was synthesized in a manner similar to Example 88, with 4-(2-(1H-indazol-6-yl)-1-((2-(trimethylsilyl)ethoxy)methyl)-1H-pyrrolo[2,3-b]pyridin-4-yl)pyridin-2-amine (112) instead of 4-(2-(1H-pyrazol-4-yl)-1-((2-(trimethylsilyl)ethoxy)methyl)-1H-pyrrolo[2,3-b]pyridin-4-yl)pyridin-2-amine (106) as substrate, and without treatment with basic ion exchange Bio-Rad® AG 1-x8 resin.

¹H-NMR (400 MHz, d₆-dmso): δ 13.30 (s, 1H), 12.50 (s, 1H), 8.31 (d, 1H), 8.16 (m, 1H), 8.09 (m, 2H), 7.85 (d, 1H), 7.75 (m, 1H), 7.20 (m, 2H), 6.93 (m, 2H), 6.11 (m, 1H). MS (ES) for C₁₉H₁₄N₆ found 327 (MH+).

### Example 92

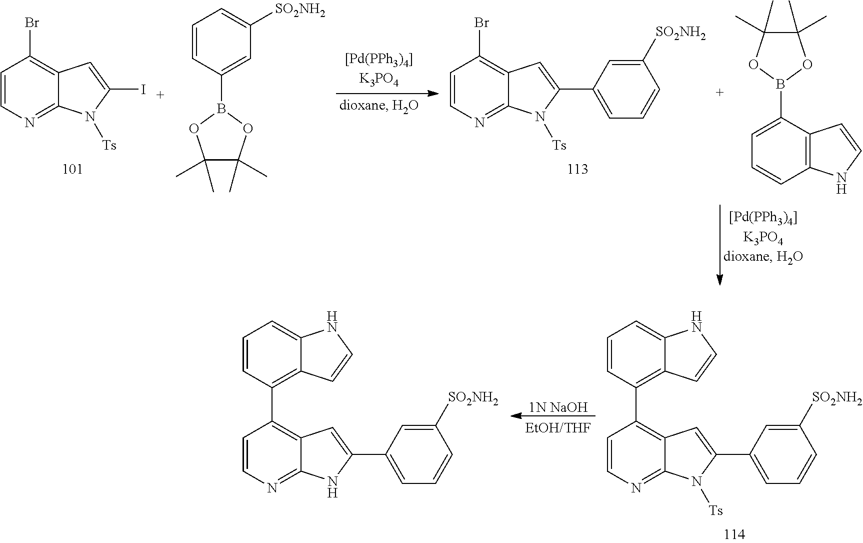

### 3-(4-Bromo-1-tosyl-1H-pyrrolo[2,3-b]pyridin-2-yl)benzenesulfonamide (113)

The title compound was synthesized in a manner similar to Example 87, with 3-(4,4,5,5-tetramethyl-1,3,2-dioxaborolan-2-yl)benzenesulfonamide instead of 4-(4,4,5,5-tetramethyl-1,3,2-dioxaborolan-2-yl)pyridin-2-amine as reagent, tetrakis(triphenylphosphine)palladium(0) instead of 1,1′-bis(diphenylphosphino)ferrocene-palladium(11) dichloride dichloromethane adduct as catalyst, and dioxane instead of DME as solvent.

¹H-NMR (400 MHz, CD₃OD): δ 8.26 (m, 1H), 8.10 (m, 1H), 7.77 (m, 1H), 7.71 (d, 2H), 7.66 (m, 2H), 7.48 (d, 1H), 7.25 (d, 2H), 6.69 (s, 1H), 2.36 (s, 3H). MS (ES) for C₂₀H₁₆BrN₃O₄S, found 506, 508 (MH+).

### 3-(4-(1H-Indol-4-yl)-1-tosyl-1H-pyrrolo[2,3-b]pyridin-2-yl)benzenesulfonamide (114)

The title compound was synthesized in a manner similar to Example 87, with 3-(4-bromo-1-tosyl-1H-pyrrolo[2,3-b]pyridin-2-yl)benzenesulfonamide (113) instead of 4-bromo-2-iodo-1-tosyl-1H-pyrrolo[2,3-b]pyridine (101) as substrate, 4-(4,4,5,5-tetramethyl-1,3,2-dioxaborolan-2-yl)-1H-indoleacid instead of 4-(4,4,5,5-tetramethyl-1,3,2-dioxaborolan-2-yl)pyridin-2-amine as reagent, tetrakis(triphenylphosphine)palladium(0) instead of 1,1′-bis(diphenylphosphino)ferrocene-palladium(11) dichloride dichloromethane adduct as catalyst, and dioxane instead of DME as solvent. The compound was only partly purified and characterized, but deemed suitable to undergo the next preparation.

MS (ES) for C₂₈H₂₂N₄O₄S₂, found 543 (MH+).

### 3-[4-(1H-Indol-4-yl)-1H-pyrrolo[2,3-b]pyridin-2-yl]benzenesulfonamide

The title compound was synthesized in a manner similar to Example 63, with 3-(4-(1H-indol-4-yl)-1-tosyl-1H-pyrrolo[2,3-b]pyridin-2-yl)benzenesulfonamide (114) instead of (S)-4-(1-(phenylsulfonyl)-4-(3-(3,3,3-trifluoropropylamino)pyrrolidin-1-yl)-1H-pyrrolo[2,3-b]pyridin-3-yl)pyrimidin-2-amine (46) as substrate, NaOH instead of LiOH, and by heating the reaction mixture at 60° C. for 6 h, instead of rt.

¹H-NMR (400 MHz, CD₃OD): δ 8.38 (m, 1H), 8.05 (m, 1H), 7.95 (m, 2H), 7.74 (m, 1H), 7.67 (m, 3H), 7.47 (m, 1H), 7.36-7.42 (m, 2H), 7.22 (s, 1H), 6.59 (m, 1H). MS (ES) for C₂₁H₁₆N₁₄O₂S, found 389 (MH+).

### Example 93

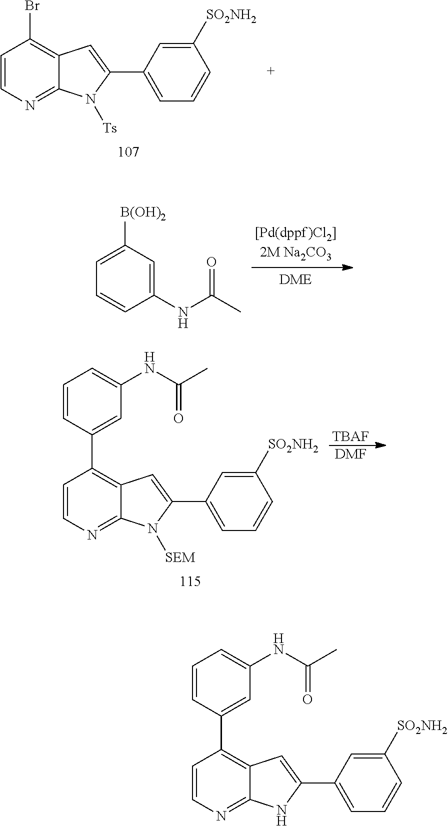

### N-(3-(2-(3-Sulfamoylphenyl)-1-((2-(trimethylsilyl)ethoxy)methyl)-1H-pyrrolo[2,3-b]pyridin-4-yl)Phenyl)acetamide (115)

The title compound was synthesized in a manner similar to Example 88, with 3-(4-bromo-1-tosyl-1H-pyrrolo[2,3-b]pyridin-2-yl)benzenesulfonamide (107) instead of 4-bromo-2-(1H-pyrazol-4-yl)-14(2-(trimethylsilyl)ethoxy)methyl)-1H-pyrrolo[2,3-b]pyridine (105) as substrate, and with 3-acetamidophenylboronic acid instead of 4-(4,4,5,5-tetramethyl-1,3,2-dioxaborolan-2-yl)pyridin-2-amine as reagent.

¹H-NMR (400 MHz, CD₃OD): δ 8.41 (m, 1H), 8.36 (m, 1H), 8.18 (m, 1H), 8.08 (m, 1H), 8.00 (m, 1H), 7.68 (m, 1H), 7.57 (m, 1H), 7.52 (m, 2H), 7.33 (m, 1H), 7.04 (s, 1H), 5.74 (s, 2H), 3.77 (m, 2H), 1.03 (m, 2H), 0.00 (s, 9H). MS (ES) for C₂₇H₃₂N₄O₄SSi, found 537 (MH+).

### N-(3-{2-[3-(Aminosulfonyl)phenyl]-1H-pyrrolo[2,3-b]pyridin-4-yl}phenyl)acetamide

A 100 mL round bottom flask was charged with N-(3-(2-(3-sulfamoylphenyl)-14(2-(trimethylsilyl)ethoxy)methyl)-1H-pyrrolo[2,3-b]pyridin-4-yl)phenyl)acetamide (115, 77 mg, 0.144 mmol), tetra-n-butylammonium fluoride trihydrate (282 mg, 0.893 mmol), and anhydrous DMF (5 mL). A distillation head was connected. The mixture was concentrated to dryness upon stirring and heating up to 35° C. under vacuum (<1 mmHg). The residual gum was kept under vacuum at rt for another 6 h. The crude reaction material was dissolved in ethyl acetate and the solution was washed twice with water, then with brine, finally dried over MgSO₄. After purification by flash chromatography (95:5 dichloromethane/methanol to 92:7:1 dichloromethane/methanol/28% (w/w) ammonium hydroxide), the title compound was obtained a solid (41 mg, 70% yield).

¹H-NMR (400 MHz, d₆-dmso): δ 12.54 (s, 1H), 10.17 (s, 1H), 8.41 (s, 1H), 8.33 (d, 1H), 8.17 (d, 1H), 8.06 (s, 1H), 7.74 (m, 3H), 7.49 (m, 4H), 7.19 (m, 2H), 2.09 (s, 3H). MS (ES) for C₂₁H₁₈N₄O₃S, found 407 (MH+).

### Example 94

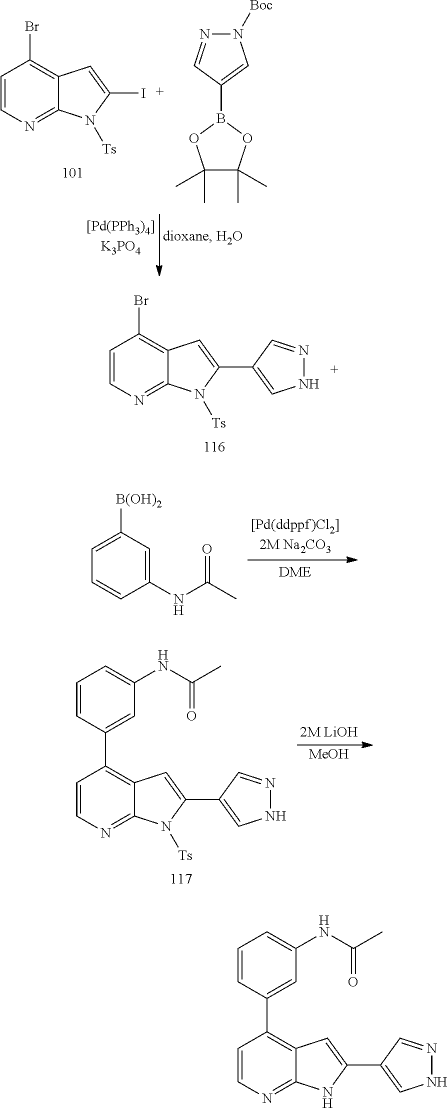

### 4-Bromo-2-(1H-pyrazol-4-yl)-1-tosyl-1H-pyrrolo[2,3-b]pyridine (116)

The title compound was synthesized in a manner similar to Example 88, with compound 101 instead of compound 104 as substrate. The product was deemed pure enough to undergo the next preparation without further characterization.

MS (ES) for C₁₇H₁₃BrN₄O₂S, found 417, 419 (MH+).

### N-(3-(2-(1H-Pyrazol-4-yl)-1-tosyl-1H-pyrrolo[2,3-b]pyridin-4-yl)phenyl)acetamide (117)

The title compound was synthesized in a manner similar to Example 88, with 4-bromo-2-(1H-pyrazol-4-yl)-1-tosyl-1H-pyrrolo[2,3-b]pyridine (116) instead of 4-bromo-2-(1H-pyrazol-4-yl)-14(2-(trimethylsilyl)ethoxy)methyl)-1H-pyrrolo[2,3-b]pyridine (105) as substrate, and with 3-acetamidophenylboronic acid instead of 4-(4,4,5,5-tetramethyl-1,3,2-dioxaborolan-2-yl)pyridin-2-amine as reagent.

¹H-NMR (400 MHz, CD₃OD): δ 8.42 (d, 1H), 7.96 (m, 1H), 7.84 (m, 1H), 7.76 (m, 1H), 7.62 (m, 2H), 7.54 (m, 1H), 7.43 (m, 1H), 7.32 (m, 3H), 7.19 (m, 1H), 6.80 (s, 1H), 2.34 (s, 3H), 2.18 (s, 3H). MS (ES) for C₂₅H₂₁N₅O₃S, found 472 (MH+).

### N-{3-[2-(1H-Pyrazol-4-yl)-1H-pyrrolo[2,3-b]pyridin-4-yl]phenyl}acetamide

The title compound was synthesized in a manner similar to Example 63, with N-(3-(2-(1H-pyrazol-4-yl)-1-tosyl-1H-pyrrolo[2,3-b]pyridin-4-yl)phenyl)acetamide (117) instead of (S)-4-(1-(phenylsulfonyl)-4-(3-(3,3,3-trifluoropropylamino)pyrrolidin-1-yl)-1H-pyrrolo[2,3-b]pyridin-3-yl)pyrimidin-2-amine (46) as substrate, and by heating the reaction mixture at 60° C. for 6 h, instead of rt.

¹H-NMR (400 MHz, CDCl₃/CD₃OD): δ 8.16 (d, 1H), 8.07 (m, 3H), 7.73 (d, 1H), 7.62 (m, 1H), 7.50 (m, 1H), 7.21 (m, 1H), 7.18 (d, 1H), 6.85 (s, 1H), 2.22 (s, 3H). MS (ES) for C₁₈H₁₅N₅O, found 318 (MH+).

### Example 95

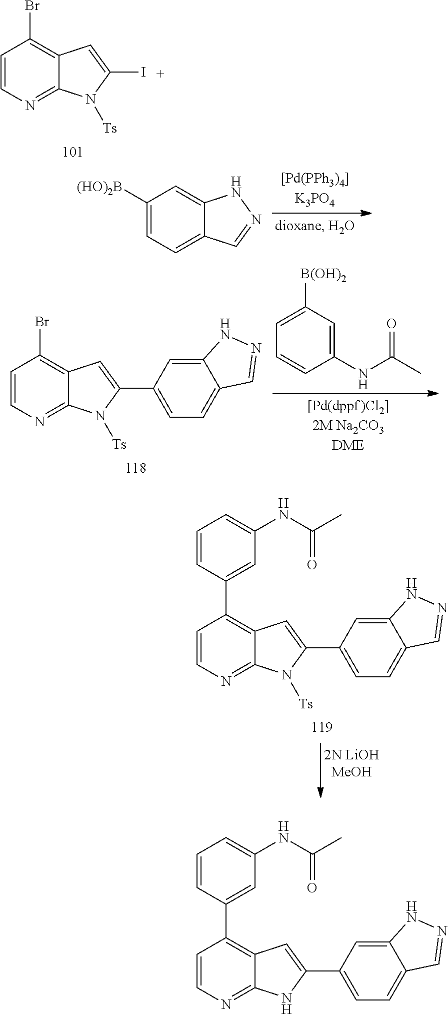

### 6-(4-Bromo-1-tosyl-1H-pyrrolo[2,3-b]pyridin-2-yl)-1H-indazole (118)

The title compound was synthesized in a manner similar to Example 91, with compound 101 instead of compound 104 as substrate. The product was deemed pure enough to undergo the next preparation without further characterization.

MS (ES) for C₂₉H₂₃BrN₅O₃S, found 467, 469 (MH+).

### N-(3-(2-(1H-Indazol-6-yl)-1-tosyl-1H-pyrrolo[2,3-b]pyridin-4-yl)phenyl)acetamide (119)

The title compound was synthesized in a manner similar to Example 88, with 6-(4-bromo-1-tosyl-1H-pyrrolo[2,3-b]pyridin-2-yl)-1H-indazole (118) instead of 4-bromo-2-(1H-pyrazol-4-yl)-14(2-(trimethylsilyl)ethoxy)methyl)-1H-pyrrolo[2,3-b]pyridine (105) as substrate, and with 3-acetamidophenylboronic acid instead of 4-(4,4,5,5-tetramethyl-1,3,2-dioxaborolan-2-yl)pyridin-2-amine as reagent.

¹H-NMR (400 MHz, CD₃OD): δ 8.45 (d, 1H), 8.11 (d, 1H), 8.01 (m, 1H), 7.82 (d, 1H), 7.73 (m, 1H), 7.68 (m, 2H), 7.44 (m, 3H), 7.37 (m, 2H), 7.22 (m, 2H), 6.92 (s, 1H), 2.35 (s, 3H), 2.14 (s, 3H). MS (ES) for C₂₉H₂₃N₅O₃S, found 522 (MH+).

### N-{3-[2-(1H-Indazol-6-yl)-1H-pyrrolo[2,3-b]pyridin-4-yl]phenyl}acetamide

The title compound was synthesized in a manner similar to Example 63, with N-(3-(2-(1H-indazol-6-yl)-1-tosyl-1H-pyrrolo[2,3-b]pyridin-4-yl)phenyl)acetamide (119) instead of (S)-4-(1-(phenylsulfonyl)-4-(3-(3,3,3-trifluoropropylamino)pyrrolidin-1-yl)-1H-pyrrolo[2,3-b]pyridin-3-yl)pyrimidin-2-amine (46) as substrate, and by heating the reaction mixture at 60° C. for 6 h, instead of rt.

¹H-NMR (400 MHz, d₆-dmso): δ 13.30 (br s, 1H), 12.45 (br s, 1H), 10.17 (br s, 1H), 8.29 (d, 1H), 8.14 (m, 2H), 7.84 (d, 1H), 7.72 (m, 2H), 7.50 (m, 2H), 7.22 (m, 2H), 6.98 (s, 1H), 2.50 (s, 3H). MS (ES) for C₂₂H₁₇N₅O, found 368 (MH+).

### Example 96

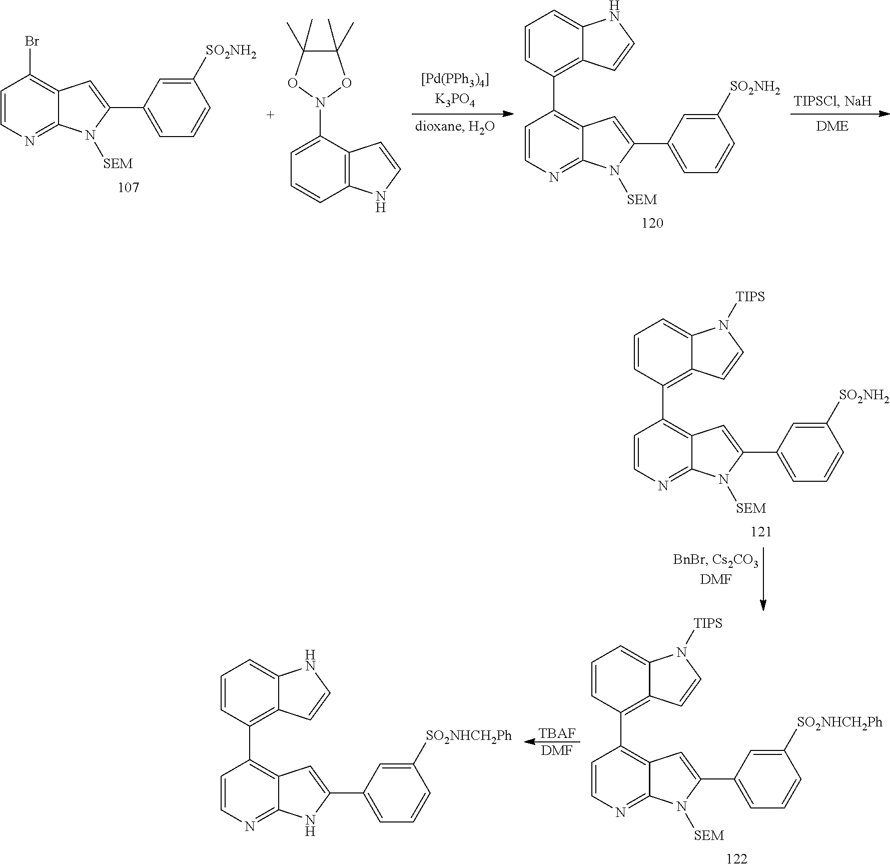

### 3-(4-(1H-Indol-4-yl)-14(2-(trimethylsilyl)ethoxy)methyl)-1H-pyrrolo[2,3-b]pyridin-2-yl)benzenesulfonamide (120)

The title compound was synthesized in a manner similar to Example 97, with 3-(4-bromo-1-((2-(trimethylsilyl)ethoxy)methyl)-1H-pyrrolo[2,3-b]pyridin-2-yl)benzenesulfonamide (107) instead of 4-bromo-2-iodo-1-tosyl-1H-pyrrolo[2,3-b]pyridine (101) as substrate, 4-(4,4,5,5-tetramethyl-1,3,2-dioxaborolan-2-yl)-1H-indoleacid instead of 4-(4,4,5,5-tetramethyl-1,3,2-dioxaborolan-2-yl)pyridin-2-amine as reagent, tetrakis(triphenylphosphine)palladium(0) instead of 1,1′-bis(diphenylphosphino)ferrocene-palladium(II) dichloride dichloromethane adduct as catalyst, and dioxane instead of DME as solvent.

¹H-NMR (400 MHz, CDCl₃): δ 8.52 (br s, 1H), 8.48 (d, 1H), 8.32 (s, 1H), 8.03 (m, 1H), 7.92 (m, 2H), 7.49-7.58 (m, 3H), 7.41 (m, 2H), 7.34 (d, 1H), 7.27 (m, 1H), 6.75 (s, 1H), 6.61 (m, 1H), 5.72 (s, 2H), 3.84 (m, 2H), 1.04 (m, 2H), 0.00 (s, 9H). MS (ES) for C₂₇H₃₀N₄O₃SSi, found 519 (MH+).

### 3-(4-(1-(Triisopropylsilyl)-1H-indol-4-yl)-1-((2-(trimethylsilyl)ethoxy)methyl)-1H-pyrrolo[2,3-b]pyridin-2-yl)benzenesulfonamide (121)

The title compound was synthesized in a manner similar to Example 76, with 3-(4-(1H-indol-4-yl)-1-((2-(trimethylsilyl)ethoxy)methyl)-1H-pyrrolo[2,3-b]pyridin-2-yl)benzenesulfonamide (120) instead of 4-iodo-1H-pyrrolo[2,3-b]pyridine as substrate, and with TIPSCI instead of TsCI as reagent.

¹H-NMR (400 MHz, CDCl₃): δ 8.64 (m, 1H), 8.48 (d, 1H), 8.32 (m, 1H), 7.97 (m, 1H), 7.91 (m, 1H), 7.50 (m, 2H), 7.41 (m, 2H), 7.32 (m, 1H), 7.25 (m, 1H), 6.72 (s, 1H), 6.61 (m, 1H), 5.70 (s, 2H), 3.83 (m, 2H), 1.24-1.30 (m, 5H), 1.05 (d, 18H), 0.00 (s, 9H). MS (ES) for C₃₆H₅₀N₄O₃SSi₂, found 676 (MH+).

### N-Benzyl-3-(4-(1-(triisopropylsilyl)-1H-indol-4-yl)-1-((2-(trimethylsilyl)ethoxy)methyl)-1H-pyrrolo[2,3-b]pyridin-2-yl)benzenesulfonamide (122)

A solution of 3-(4-(1-(triisopropylsilyl)-1H-indol-4-yl)-14(2-(trimethylsilyl)ethoxy)methyl)-1H-pyrrolo[2,3-b]pyridin-2-yl)benzenesulfonamide (121, 66 mg, 0.098 mmol) in anhydrous DMF (5 mL) was added to a suspension of Cs₂CO₃ (153 mg, 0.47 mmol) in DMF at rt. Benzyl bromide (0.065 mL, 0.55 mmol) was added dropwise via syringe in four different portions. The mixture was heated at 60° C. for 18 h. The title compound was detected by LC-MS as major product in the mixture, which was directly treated as described in the next preparation.

### 3-[4-(1H-Indol-4-yl)-1H-pyrrolo[2,3-b]pyridin-2-yl]-N-(phenylmethyl)benzenesulfonamide

The title compound was synthesized in a manner similar to Example 93, with the crude reaction mixture containing N-benzyl-3-(4-(1-(triisopropylsilyl)-1H-indol-4-O-1-((2-(trimethylsilyl)ethoxy)methyl)-1H-pyrrolo[2,3-b]pyridin-2-yl)benzenesulfonamide (122) instead of N-(3-(2-(3-sulfamoylphenyl)-1-((2-(trimethylsilyl)ethoxy)methyl)-1H-pyrrolo[2,3-b]pyridin-4-yl)phenyl)acetamide (115) as substrate.

¹H-NMR (400 MHz, CDCl₃): δ 8.31 (m, 1H), 8.21 (d, 1H), 7.74 (dd, 2H), 7.27-7.35 (m, 8H), 7.13 (m, 3H), 6.78 (s, 1H), 6.54 (d, 1H), 5.33 (s, 2H). MS (ES) for C₂₈H₂₂N₄O₂S, found 479 (MH+).

### Example 97

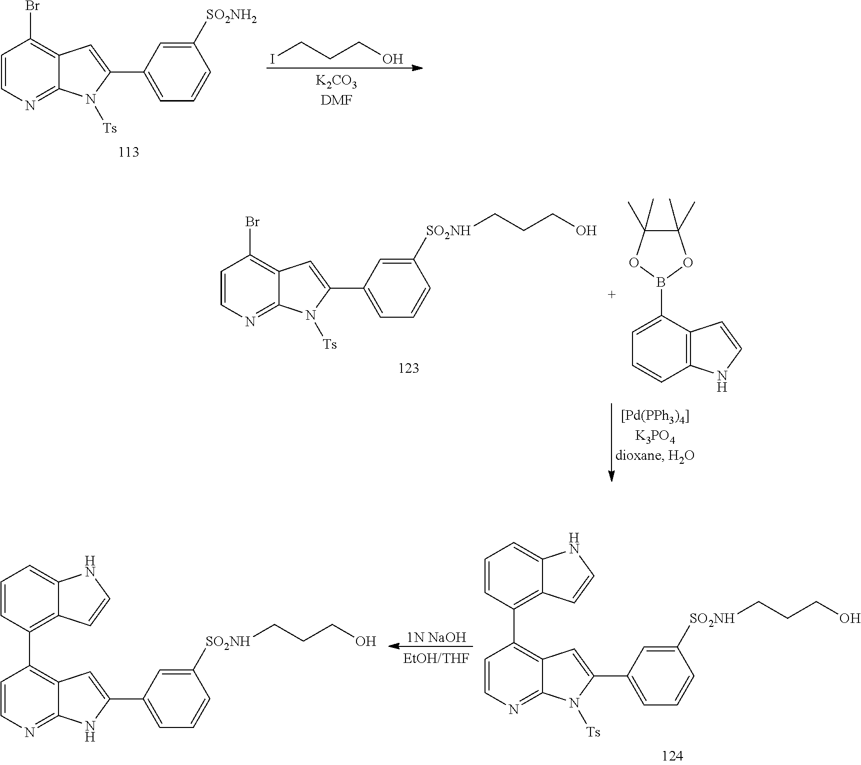

### 3-(4-Bromo-1-tosyl-1H-pyrrolo[2,3-b]pyridin-2-yl)-N-(3-hydroxypropyl)benzenesulfonamide (123)

The title compound was synthesized in a manner similar to Example 96, with 3-(4-bromo-1-tosyl-1H-pyrrolo[2,3-b]pyridin-2-yl)benzenesulfonamide (113) instead of 3-(4-(1-(triisopropylsilyl)-1H-indol-4-yl)-14(2-(trimethylsilyl)ethoxy)methyl)-1H-pyrrolo[2,3-b]pyridin-2-yl)benzenesulfonamide (121) as substrate, 3-iodopropanol and potassium carbonate instead of benzyl bromide and cesium carbonate as reagents.

¹H-NMR (400 MHz, CDCl₃): δ 8.30 (m, 1H), 8.08 (m, 1H), 7.99 (m, 1H), 7.78 (m, 3H), 7.65 (m, 1H), 7.41 (d, 1H), 7.22 (m, 2H), 6.65 (s, 1H), 5.28 (m, 1H), 3.74 (m, 2H), 3.23 (m, 2H), 2.36 (s, 3H), 1.74 (m, 2H). MS (ES) for C₂₃H₂₂BrN₃O₅S₂, found 564, 566 (MH+).

### 3-(4-(1H-Indol-4-yl)-1-tosyl-1H-pyrrolo[2,3-b]pyridin-2-yl)-N-(3-hydroxypropyl)benzenesulfonamide (124)

The title compound was synthesized in a manner similar to Example 87, with 3-(4-bromo-1-tosyl-1H-pyrrolo[2,3-b]pyridin-2-yl)-N-(3-hydroxypropyl)benzenesulfonamide (123) instead of 4-bromo-2-iodo-1-tosyl-1H-pyrrolo[2,3-b]pyridine (101) as substrate, 4-(4,4,5,5-tetramethyl-1, 3,2-dioxaborolan-2-yl)-1H-indoleacid instead of 4-(4,4,5,5-tetramethyl-1,3,2-dioxaborolan-2-yl)pyridin-2-amine as reagent, tetrakis(triphenylphosphine)palladium(0) instead of 1,1′-bis(diphenylphosphino)ferrocene-palladium(II) dichloride dichloromethane adduct as catalyst, and dioxane instead of DME as solvent.

¹H-NMR (400 MHz, CDCl₃): δ 8.57 (d, 1H), 8.45 (m, 1H), 8.08 (m, 1H), 7.93 (m, 1H), 7.83 (m, 2H), 7.71 (m, 1H), 7.58 (m, 1H), 7.48 (m, 2H), 7.21-7.31 (m, 6H), 6.67 (s, 1H), 6.44 (m, 1H), 5.31 (m, 1H), 3.71 (m, 2H), 3.22 (m, 2H), 2.37 (s, 3H), 1.70 (m, 2H). MS (ES) for C₃₁H₂₈N₁₄O₅S₂, found 601 (MH+).

### 3-[4-(1H-Indol-4-yl)-1H-pyrrolo[2,3-b]pyridin-2-yl]-N-(3-hydroxypropyl)benzenesulfonamide

The title compound was synthesized in a manner similar to Example 63, with 3-(4-(1H-indol-4-yl)-1-tosyl-1H-pyrrolo[2,3-b]pyridin-2-yl)-N-(3-hydroxypropyl)benzenesulfonamide (124) instead of (S)-4-(1-(phenylsulfonyl)-4-(3-(3,3,3-trifluoropropylamino)pyrrolidin-1-yl)-1H-pyrrolo[2,3-b]pyridin-3-yl)pyrimidin-2-amine (46) as substrate, NaOH instead of LiOH, and by heating the reaction mixture at 60° C. for 6 h, instead of rt.

¹H-NMR (400 MHz, CDCl₃/CD₃OD): δ 8.31 (m, 1H), 8.27 (m, 1H), 8.02 (m, 1H), 7.80 (m, 1H), 7.59 (m, 2H), 7.40 (m, 2H), 7.32 (m, 2H), 7.00 (s, 1H), 6.58 (m, 1H), 3.62 (m, 2H), 3.04 (m, 2H), 1.71 (m, 2H). MS (ES) for C₂₄H₂₂N₄O₃S, found 447 (MH+).

Using analogous synthetic techniques as in the above examples, and substituting with the appropriate alternative starting materials, the following compounds were prepared. Alternative starting materials were obtained commercially unless otherwise indicated.

### (4S)-1-[3-(2-aminopyrimidin-4-yl)-1H-pyrrolo[2,3-b]pyridin-4-yl]-4-fluoro-L-proline

MS (ES) for C₁₆H₁₅FN₆O₂, found 343.2 (MH+).

### (3S)-1-[3-(2-aminopyrimidin-4-yl)-1H-pyrrolo[2,3-b]pyridin-4-yl]pyrrolidine-3-carboxylic acid

MS (ES) for C₁₆H₁₆N₆O₂, found 325.2 (MH+).

### 2-{4-[3-(2-aminopyrimidin-4-yl)-1H-pyrrolo[2,3-b]pyridin-4-yl]-2-oxopiperazin-1-yl}-N-(2-methylpropyl)acetamide

MS (ES) for C₂₁H₂₆N₈O₂, found 423.2 (MH+).

### 4-{4-[(2S)-2-(pyrrolidin-1-ylmethyl)pyrrolidin-1-yl]-1H-pyrrolo[2,3-b]pyridin-3-yl}pyrimidin-2-amine

¹H-NMR (400 MHz, d6-DMSO): δ 11.86 (s, 1H), 8.15-8.14 (d, 1H), 7.98-7.97 (d, 1H), 7.62 (s, 1H), 6.75-6.74 (d, 1H), 6.57-6.55 (d, 1H), 6.42 (s, 2H), 3.85-3.79 (m, 1H), 3.25-3.18 (m, 1H), 3.01-2.96 (m, 1H), 2.45-2.21 (m, 6H), 2.03-1.95 (m, 1H), 1.77-1.65 (m, 3H), 1.62-1.56 (s, 4H). MS (ES), C₂₀H₂₅N₇ found 364.0 (MH+).

### 1-[3-(2-aminopyrimidin-4-yl)-1H-pyrrolo[2,3-b]pyridin-4-yl]pyrrolidin-2-one

¹H-NMR (400 MHz, MeOH-d₄): δ 8.32 (d, 1H), 8.17 (d, 1H), 7.88 (s, 1H), 7.15 (d, 1H), 6.85 (d, 1H), 4.11 (t, 2H), 2.41 (t, 2H), 2.23-2.34 (m, 2H). MS (ES) for C₁₅H₁₄N₆O, found 295.2 (MH⁺).

### 4-[4-chloro-6-(methyloxy)-3H-pyrrolo[2,3-b]pyridin-3-yl]pyrimidin-2-amine

MS (ES) for C₁₂H₁₀ClN₅O, found 276.2 (MH+).

### {(2S)-1-[3-(2-aminopyrimidin-4-yl)-6-(methyloxy)-1H-pyrrolo[2,3-b]pyridin-4-yl]pyrrolidin-2-yl}methanol

MS (ES) for C₁₇H₂₀N₆O₂, found 341.1 (MH+).

### 4-(4-{(3R)-3[(1-methylethyl)oxy]pyrrolidin-1-yl}-1H-pyrrolo[2,3-b]pyridin-3-yl)pyrimidin-2-amine

MS (ES) for C₁₈H₂₂N₆O, found 339.2 (MH+).

### 5-[3-(2-aminopyrimidin-4-yl)-1H-pyrrolo[2,3-b]pyridin-4-yl]pyridin-2-ol

MS (ES) for C₁₆H₁₂N₆O, found 305.1 (MH+).

### 4-{4-[4-(methyloxy)-1H-pyrrolo[3,2-c]pyridin-2-yl]-1H-pyrrolo[2,3-b]pyridin-3-yl}pyrimidin-2-amine

MS (ES) for C₁₉H₁₅N₇O, found 358.1 (MH+).

### {(2S)-1-[3-(2-amino-1,3-thiazol-4-yl)-1H-pyrrolo[2,3-b]pyridin-4-yl]pyrrolidin-2-yl}methanol

MS (ES) for C₁₅H₁₇N₅OS, found 316.3 (MH+).

### 4-(methyloxy)-2-(1H-pyrrolo[2,3-b]pyridin-4-yl)-1H-pyrrolo[3,2-c]pyridine

MS (ES) for C₁₅H₁₂N₄O, found 265.1 (MH+).

### -4-yl)-5-fluoro-1H-pyrrolo[2,3-b]pyridin-4-yl](phenyl)methanone

¹H-NMR (400 MHz, CDCl₃/CD₃OD): δ 8.46 (s, 1H), 8.39 (m, 1H), 7.89 (m, 3H), 7.67 (m, 1H), 7.53 (t, 2H), 7.12 (d, 1H). MS (ES) for C₁₈H₁₂FN₅O, found 334.1 (MH+).

### CDK9 Luciferase-Coupled Chemiluminescence Assay Protocol

CDK9 activity is measured as the percent of ATP consumed following the kinase reaction using luciferase-luciferin-coupled chemiluminescence. The kinase reaction consists of active CDK9/cyclinT1 (Millipore) phosphorylating the serine-2 residue of the RNA polymerase II c-terminal domain repeat YSPTSPS peptide (Anaspec). All reactions were conducted in 384-well white, medium binding microtiter plates (Greiner). Kinase reactions were initiated by combining test compounds (at varying concentrations), ATP, substrate, and kinase in a 20 μL volume. The standard assay concentrations for enzyme, ATP, and substrate are 10 nM, 0.5 μM, and 45 μM, respectively. The assay buffer is composed of 20 mM Tris-HCL (pH 7.5), 10 mM MgCl₂, 3 mM MnCl₂, 0.01% Triton X-100, and 1 mM DTT. The reaction mixture was incubated at ambient temperature for 3 h. Following the kinase reaction, a 10 μL aliquot of luciferase-luciferin mix (Promega Kinase-Glo) was added and the chemiluminescence signal measured using a Victor2 plate reader (Perkin Elmer). Total ATP consumption was limited to 40-60%. IC₅₀ values were calculated by nonlinear regression analysis using the four-parameter equation [Y=min+(max−min)/(1+(X/IC₅₀)^{N})] where Y is the observed signal, X is the inhibitor concentration, min is the background signal in the absence of enzyme (0% enzyme activity), max is the signal in the absence of inhibitor (100% enzyme activity), IC₅₀ is the inhibitor concentration at 50% enzyme inhibition and N represents the empirical Hill slope as a measure of cooperativity. Typically N should approximate unity. Curve fitting was performed using commercial software (idbs ActivityBase). All of the compounds in Table 1 have CDK9 IC₅₀ values of less than 2,000 nm.

One way of being able to determine CDK8 IC₅₀ values is by using the LanthaScreen™ Eu kinase binding assay for CDK8/cyclin C available from Invitrogen located in Carlsbad, Calif. 92008.

We claim:

1. A compound according to Formula I: 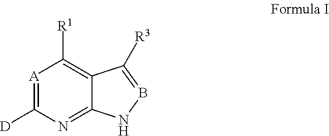
or a pharmaceutically-acceptable salt thereof, wherein:
R¹ is selected from H, halo, —NR⁴R⁵, —(C₁-C₄)alky-NR⁸R⁹, —O—(C₁-C₄)alky-NR⁸R⁹, heteroaryl optionally substituted with amino, hydroxyl, alkoxyl or aryl-(C₁-C₃)alkyl, (5-6 membered)heterocycloalkyl optionally substituted with hydroxyl, amino, alkynyl substituted with hydroxyl or —(CH₂)₀₋₃—NR⁸R⁹, phenyl substituted with —CF₃ or cyano, alkyl substituted with hydroxyl, and R^{xa};
R², when A is CR², is selected from H, halo, alkyl, —NR⁸R⁹, alkoxy, alkoxyalkoxy, dialkylaminoalkoxy, and phenyl; or R¹ and R², together with the atoms to which they are attached, join together to form a heterocycloalkyl or heteroaryl group, wherein the heterocycloalkyl or heteroaryl groups are each optionally substituted with arylalkyl or arylalkoxyalkoxy;
R³ is a (5-9 membered)heteroaryl or phenyl, wherein the (5-9 membered)heteroaryl or phenyl are each optionally substituted with 1 or 2 substituents independently selected from amine, alkoxy, hydroxyl, alkyl, aminocarbonyl and halo;
A is selected from N and CR²;
B is independently selected from N, CH and CR^{Xb};
D is H or OH;
R⁴ is selected from H, alkyl optionally substituted with 1, 2 or 3 groups independently selected from hydroxyl, amino, halo, alkoxy, alkylamino, dialkylamino and dialkylaminoalkyl, alkoxycarbonyl, alkoxyalkyl, oxo, —CN, dialkylaminocarbonylalkyl optionally substituted with hydroxyl, cycloalkyl optionally substituted with amine, aryl optionally substituted with amine, arylalkyl, heteroarylalkyl optionally substituted with alkyl, heterocycloalkylalkyl and heterocycloalkyl optionally substituted with 1, 22 or 3 substituents independently selected from alkyl, hydroxyl, amino, and hydroxyalkyl;
R⁵ is selected from alkyl optionally substituted with 1, 2 or 3 groups independently selected from hydroxyl, amino, halo, alkoxy, alkylamino, dialkylamino and dialkylaminoalkyl, alkoxycarbonyl, alkoxyalkyl, oxo, —CN, dialkylaminocarbonylalkyl optionally substituted with hydroxyl, cycloalkyl optionally substituted with amine, aryl optionally substituted with amine, arylalkyl, heteroarylalkyl optionally substituted with alkyl, heterocycloalkylalkyl and heterocycloalkyl optionally substituted with 1, 2 or 3 substituents independently selected from alkyl, hydroxyl, amino, and hydroxyalkyl;
or R⁴ and R⁵, together with the nitrogen to which they are both attached, join together to form a heterocycloalkyl or heteroaryl group, wherein the heteroaryl group is optionally substituted with 1-3 substituents selected from alkyl, halo alkoxy, amino, alkylamino, dialkylamino, aminoalkyl, aminoalkylamino, dialkylaminoalkyl, hydroxyl, hydroxyalkyl, —CF₃, alkylcarbonylamino, —C(O)—N(R⁸)R⁹ and —S(O₂)—NH₂, and the heterocycloalkyl group is optionally substituted with 1, 2 or 3 substituents independently selected from halo, alkyl optionally substituted with 1-3 halo, carboxyalkyl, amino, hydroxyl, hydroxyalkyl, alkoxy optionally substituted with 1-3 halo, alkoxyalkyl optionally substituted with 1-3 halo, oxo,
—(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with —NR⁸R⁹, —(CH₂)₀₋₃—N(R⁶)R⁷,
—C(O)—N(R⁸)R⁹, alkyl-C(O)N(H)(alkyl)-, aryl, arylalkyl optionally substituted with 1 or 2 groups selected from halo and alkyl, alkoxycarbonyl and aminoalkyl;
provided that the optionally substituted heterocycloalkyl group cannot be substituted with more than one group selected from —(CH₂)₀₋₃-heterocycloalkyl optionally substituted with alkyl, ═N(H)—OH, —S(O₂)—NH₂, oxo,
—N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with alkyl, (CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, and alkoxycarbonyl,
and provided that the optionally substituted heteroaryl group cannot be substituted with more than one alkylamino, dialkylamino, aminoalkyl, aminoalkylamino, dialkylaminoalkyl, alkylcarbonylamino, —C(O)—N(R⁸)R⁹ and —S(O₂)—NH₂;
R⁶ is selected from H and alkyl;
R⁷ is selected from H, hydroxyalkyl, alkylaminoalkylcarbonyl, dialkylaminoalkylcarbonyl, and alkyl optionally substituted with 1-3 halo;
R⁸ is selected from H and alkyl;
R⁹ is selected from H and alkyl;
R¹⁰ is selected from H, halo and alkyl;
R^{xa} is selected from halo, aryl or heteroaryl, wherein said aryl or heteroaryl is optionally substituted with a group selected from amino, aminocarbonyl, alkylaminocarbonyl, alkylcarbonylamino, dialkylaminocarbonyl, alkyl, haloalkyl, —S(O₂)—NH₂, and hydroxyalkyl;
R^{xb} is an aryl or heteroaryl, wherein said aryl or heteroaryl are each optionally substituted with a group selected from amino, halo, alkyl, hydroxyalkyl and —S(O₂)—N(H)—(CH₂)₀₋₃—R^{xc}; and
R^{xc} is selected from H, OH, aryl and NH₂,
provided that R¹ can be H or halo only when either (1) R³ is pyrimidin-2-amine or thiazole optionally substituted with an amine, or (2) when A is CR² and R² is —NR⁴R⁵, amino, alkoxy, alkoxyalkoxy, dialkylaminoalkoxy or phenyl.

2. A compound according to claim 1, according to Formula IA or Formula IB: 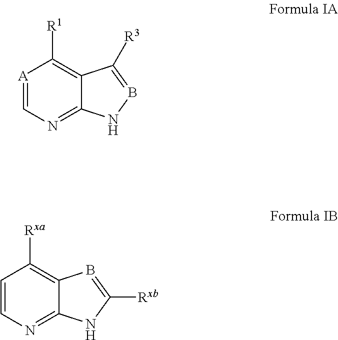
or a pharmaceutically-acceptable salt thereof, wherein:
R¹ is selected from H, halo, —NR⁴R⁵, —(C₁-C₄)alky-NR⁸R⁹, —O—(C₁-C₄)alky-NR⁸R⁹, heteroaryl optionally substituted with amino, hydroxyl, alkoxyl or aryl-(C₁-C₃)alkyl, (5-6 membered)heterocycloalkyl optionally substituted with hydroxyl, amino, alkynyl substituted with hydroxyl or —(CH₂)₀₋₃—NR⁸R⁹, phenyl substituted with —CF₃ or cyano, and alkyl substituted with hydroxyl;
R², when A is CR², is selected from H, halo, alkyl, —NR⁸R⁹, alkoxy, alkoxyalkoxy, dialkylaminoalkoxy, and phenyl;
or R¹ and R², together with the atoms to which they are attached, join together to form a heterocycloalkyl or heteroaryl group, wherein the heterocycloalkyl or heteroaryl groups are each optionally substituted with arylalkyl or arylalkoxyalkoxy;
R³ is a (5-9 membered)heteroaryl or phenyl, wherein the (5-9 membered)heteroaryl or phenyl are each optionally substituted with 1 or 2 substituents independently selected from amine, alkoxy, hydroxyl, alkyl, aminocarbonyl and halo;
A is selected from N and CR²;
B is independently selected from N and CH;
R⁴ is selected from H, alkyl optionally substituted with 1, 2 or 3 groups independently selected from hydroxyl, amino, halo, alkoxy, alkylamino, dialkylamino and dialkylaminoalkyl, alkoxycarbonyl, alkoxyalkyl, oxo, —CN, dialkylaminocarbonylalkyl optionally substituted with hydroxyl, cycloalkyl optionally substituted with amine, aryl optionally substituted with amine, arylalkyl, heteroarylalkyl optionally substituted with alkyl, heterocycloalkylalkyl and heterocycloalkyl optionally substituted with 1, 2 or 3 substituents independently selected from alkyl, hydroxyl, amino, and hydroxyalkyl;
R⁵ is selected from alkyl optionally substituted with 1, 2 or 3 groups independently selected from hydroxyl, amino, halo, alkoxy, alkylamino, dialkylamino and dialkylaminoalkyl, alkoxycarbonyl, alkoxyalkyl, oxo, —CN, dialkylaminocarbonylalkyl optionally substituted with hydroxyl, cycloalkyl optionally substituted with amine, aryl optionally substituted with amine, arylalkyl, heteroarylalkyl optionally substituted with alkyl, heterocycloalkylalkyl and heterocycloalkyl optionally substituted with 1, 2 or 3 substituents independently selected from alkyl, hydroxyl, amino, and hydroxyalkyl;
or R⁴ and R⁵, together with the nitrogen to which they are both attached, join together to form a heterocycloalkyl or heteroaryl group, wherein the heteroaryl group is optionally substituted with 1-3 substituents selected from alkyl, halo alkoxy, amino, alkylamino, dialkylamino, aminoalkyl, aminoalkylamino, dialkylaminoalkyl, hydroxyl, hydroxyalkyl, —CF₃, alkylcarbonylamino, —C(O)—N(R⁸)R⁹ and —S(O₂)—NH₂, and the heterocycloalkyl group is optionally substituted with 1, 2 or 3 substituents independently selected from halo, alkyl optionally substituted with 1-3 halo, carboxyalkyl, amino, hydroxyl, hydroxyalkyl, alkoxy optionally substituted with 1-3 halo, alkoxyalkyl optionally substituted with 1-3 halo, oxo,
—(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with —NR⁸R⁹, —(CH₂)₀₋₃—N(R⁶)R⁷,
—C(O)—N(R⁸)R⁹, alkyl-C(O)N(H)(alkyl)-, aryl, arylalkyl optionally substituted with 1 or 2 groups selected from halo and alkyl, alkoxycarbonyl and aminoalkyl; provided that the optionally substituted heterocycloalkyl group cannot be substituted with more than one group selected from —(CH₂)₀₋₃-heterocycloalkyl optionally substituted with alkyl, ═N(H)—OH, —S(O₂)—NH₂, oxo,
—N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with alkyl, (CH₂)₀₋₃—N(R⁶)R⁷, —C(O)—N(R⁸)R⁹, alkyl-2C(O)N(H)(alkyl)-, aryl, arylalkyl optionally substituted with 1 or 2 groups selected from halo and alkyl, and alkoxycarbonyl,
and provided that the optionally substituted heteroaryl group cannot be substituted with more than one alkylamino, dialkylamino, aminoalkyl, aminoalkylamino, dialkylaminoalkyl, alkylcarbonylamino, —C(O)—N(R⁸)R⁹ and —S(O₂)—NH₂;
R⁶ is selected from H and alkyl;
R⁷ is selected from H, hydroxyalkyl, alkylaminoalkylcarbonyl, dialkylaminoalkylcarbonyl, and alkyl optionally substituted with 1-3 halo;
R⁸ is selected from H and alkyl;
R⁹ is selected from H and alkyl;
R¹⁰ is selected from H, halo or alkyl;
R^{xa} is selected from halo, aryl or heteroaryl, wherein said aryl or heteroaryl is optionally substituted with a group selected from amino, aminocarbonyl, alkylaminocarbonyl, alkylcarbonylamino, dialkylaminocarbonyl, alkyl, haloalkyl, —S(O₂)—NH₂, and hydroxyalkyl;
R^{xb} is an aryl or heteroaryl, wherein said aryl or heteroaryl are each optionally substituted with a group selected from amino, halo, alkyl, hydroxyalkyl and —S(O₂)—N(H)—(CH₂)₀₋₃—R^{xc}; and
R^{xc} is selected from H, OH, aryl and NH₂,
provided that R¹ can be H or halo only when either (1) R³ is pyrimidin-2-amine or thiazole optionally substituted with an amine, or (2) when A is CR² and R² is —NR⁴R⁵, amino, alkoxy, alkoxyalkoxy, dialkylaminoalkoxy or phenyl.

3. The compound of Formula IA according to claim 2, or a pharmaceutically acceptable salt thereof, wherein R¹ is selected from —NR⁴R⁵, —(C₁-C₄)alky-NR⁸R⁹, —O—(C₁-C₄)alky-NR⁸R⁹, heteroaryl optionally substituted with amino, hydroxyl, alkoxyl or aryl-(C₁-C₃)alkyl, (5-6 membered)heterocycloalkyl optionally substituted with hydroxyl, amino, alkynyl substituted with hydroxyl or —(CH₂)₀₋₃—NR⁸R⁹, phenyl substituted with —CF₃ or cyano, and alkyl substituted with hydroxyl, wherein R⁴, R⁵, R⁸ and R⁹ are as defined in claim 1.

4. The compound of Formula IA according to claim 2, or a pharmaceutically acceptable salt thereof, wherein:
R¹ is selected from NR⁴R⁵, —(C₁-C₄)alky-NR⁸R⁹, —O—(C₁-C₄)alky-NR⁸R⁹, triazolyl optionally substituted with phenylmethyl, 1H-pyrrolo[2,3-b]pyridinyl, 1H-indolyl, pyridinyl optionally substituted with amino or methoxy, pyrazolyl, pyrimidinyl optionally substituted with amino, (5-6 membered)heterocycloalkyl optionally substituted with hydroxyl, and amino;
R³ is phenyl, pyridine or pyrimidine, wherein the phenyl, pyridine or pyrimidine is optionally substituted with 1 or 2 substituents independently selected from amine, alkoxy, hydroxyl, alkyl and aminocarbonyl;
R⁴ is selected from H, alkyl optionally substituted with 1, 2 or 3 groups independently selected from hydroxyl, amino, halo, alkoxy, alkylamino, dialkylamino and dialkylaminoalkyl, alkoxycarbonyl, alkoxyalkyl, oxo, —CN, dialkylaminocarbonylalkyl optionally substituted with hydroxyl, cycloalkyl optionally substituted with amine, aryl optionally substituted with amine, arylalkyl, heteroarylalkyl optionally substituted with alkyl, heterocycloalkylalkyl and heterocycloalkyl optionally substituted with 1, 2 or 3 substituents independently selected from alkyl, hydroxyl, amino, and hydroxyalkyl;
R⁵ is selected from alkyl optionally substituted with 1, 2 or 3 groups independently selected from hydroxyl, amino, halo, alkoxy, alkylamino, dialkylamino and dialkylaminoalkyl, alkoxycarbonyl, alkoxyalkyl, oxo, —CN, dialkylaminocarbonylalkyl optionally substituted with hydroxyl, cycloalkyl optionally substituted with amine, aryl optionally substituted with amine, arylalkyl, heteroarylalkyl optionally substituted with alkyl, heterocycloalkylalkyl and heterocycloalkyl optionally substituted with 1, 2 or 3 substituents independently selected from alkyl, hydroxyl, amino, and hydroxyalkyl;
or R⁴ and R⁵, together with the nitrogen to which they are both attached, join together to form a heteroaryl group or a heterocycloalkyl group, wherein the heteroaryl group is selected from (1) isoindolinyl, (2) 1,2,3,4-tetrahydroisoquinolinyl optionally substituted with hydroxyl, and (3) indolinyl optionally substituted with hydroxyalkyl, and wherein the heterocycloalkyl group is selected from morpholinyl, piperidinyl, piperazinyl, pyrrolidinyl, imidazolidinyl, 3-azabicyclo[3.1.0]hexyl, 1,2,3,6-tetrahydropyridinyl, hexahydrocyclopenta[c]pyrrol-2(1H)-yl, hexahydrocyclopenta[c]pyrrol-5(1H)-yl, hexahydropyrrolo[3,4-c]pyrrol-2(1H)-yl), (4aR,8aR)-octahydro-1H-pyrido[3,4-b][1,4]oxazinyl, hexahydropyrrolo[3,4-b]pyrrol-2(1H)-yl), hexahydropyrrolo[3,4-b]pyrrol-5(1H)-yl), thiazolidinyl, tetrahydro-3aH[1,3]dioxolo[4,5-c]pyrrolyl, hexahydropyrrolo[1,2-a]pyrazin-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from halo, alkyl optionally substituted with 1-3 halo, carboxyalkyl, amino, hydroxyl, hydroxyalkyl, alkoxy optionally substituted with 1-3 halo, alkoxyalkyl optionally substituted with 1-3 halo, —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, (C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with NR⁸R⁹, —(CH₂)₀₋₃—N(R⁸)R⁷, —C(O)—N(R⁸)R⁹, alkyl-C(O)N(H)(alkyl)-, aryl, arylalkyl optionally substituted with 1 or 2 groups selected from halo and alkyl, alkoxycarbonyl and aminoalkyl, provided that the optionally substituted heterocycloalkyl group cannot be substituted with more than one group selected from —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with alkyl, (CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, and alkoxycarbonyl; and
R⁶, R⁷, R⁸, R⁹, and R¹⁰ are as defined in claim 1.

5. The compound of Formula IA according to claim 2, or a pharmaceutically acceptable salt thereof, wherein:
R¹ is selected from NR⁴R⁵, —(C₁-C₄)alky-NR⁸R⁹, —O—(C₁-C₄)alky-NR⁸R⁹, triazolyl optionally substituted with phenylmethyl, 1H-pyrrolo[2,3-b]pyridinyl, 1H-indolyl, pyridinyl optionally substituted with amino or methoxy, pyrazolyl, pyrimidinyl optionally substituted with amino, (5-6 membered)heterocycloalkyl optionally substituted with hydroxyl, and amino;
R³ is phenyl, pyridine or pyrimidine, wherein the phenyl, pyridine or pyrimidine is optionally substituted with 1 or 2 substituents independently selected from alkoxy, hydroxyl, amine, alkyl and aminocarbonyl;
R⁴ is selected from H, 1-methylethyl, methyl, ethyl, propyl, 2-methylpropyl, phenylethyl, dimethylaminoethyl, methoxymethyl, piperidinyl optionally substituted with methyl, cyclopentyl, dimethylaminocarbonylmethyl, ethylpyrrolidinylmethyl, piperidinylethyl, methylaminoethyl, aminoethyl, pyrrolidinylethyl, 2-propane-1,3 diol, phenyl, carboxymethyl, dimethylaminopropyl, dimethylaminopropan-2-yl, dimethylaminopropan-1-ol-2-yl, methylaminopropyl, aminocyclohexyl, methoxyethyl, (2R)-pyrrolidinylmethyl, (2S)-pyrrolidinylmethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino, (R)-benzyl 2-amino-3-hydroxypropylcarbamate and methoxymethyl;
R⁵ is selected from is selected from propyl, 1-methylethyl, methyl, ethyl, propyl, 2-methylpropyl, phenylethyl, dimethylaminoethyl, methoxymethyl, piperidinyl optionally substituted with methyl, cyclopentyl, dimethylaminocarbonylmethyl, ethylpyrrolidinylmethyl, piperidinylethyl, methylaminoethyl, aminoethyl, pyrrolidinylethyl, 2-propane-1,3 diol, phenyl, carboxymethyl, dimethylaminopropyl, dimethylaminopropan-2-yl, dimethylaminopropan-1-ol-2-yl, methylaminopropyl, aminocyclohexyl, methoxyethyl, (2R)-pyrrolidinylmethyl, (2S)-pyrrolidinylmethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino, (R)-benzyl 2-amino-3-hydroxypropylcarbamate and methoxymethyl;
or R⁴ and R⁵, together with the nitrogen to which they are both attached, join together to form a heteroaryl group or a heterocycloalkyl group, wherein the heteroaryl group is selected from (1) isoindolinyl, (2) 1,2,3,4-tetrahydroisoquinolinyl optionally substituted with hydroxyl, and (3) indolinyl optionally substituted with hydroxyalkyl, and wherein the heterocycloalkyl group is selected from morpholinyl, piperidinyl, piperazinyl, pyrrolidinyl, imidazolidinyl, 3-azabicyclo[3.1.0]hexyl, 1,2,3,6-tetrahydropyridinyl, hexahydrocyclopenta[c]pyrrol-2(1H)-yl, hexahydrocyclopenta[c]pyrrol-5(1H)-yl, hexahydropyrrolo[3,4-c]pyrrol-2(1H)-yl), (4aR,8aR)-octahydro-1H-pyrido[3,4-b][1,4]oxazinyl, hexahydropyrrolo[3,4-b]pyrrol-2(1H)-yl), hexahydropyrrolo[3,4-b]pyrrol-5(1H)-yl), thiazolidinyl, tetrahydro-3aH[1,3]dioxolo[4,5-c]pyrrolyl, hexahydropyrrolo[1,2-a]pyrazin-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from halo, alkyl optionally substituted with 1-3 halo, carboxyalkyl, amino, hydroxyl, hydroxyalkyl, alkoxy optionally substituted with 1-3 halo, alkoxyalkyl optionally substituted with 1-3 halo, —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, (C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with NR⁸R⁹, —(CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, alkoxycarbonyl and aminoalkyl, provided that the optionally substituted heterocycloalkyl group cannot be substituted with more than one group selected from —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with alkyl, (CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, and alkoxycarbonyl; and
R⁶, R⁷, R⁸, R⁹, and R¹⁰ are as defined in claim 1.

6. The compound according to claim 5, or a pharmaceutically acceptable salt thereof, wherein:
R¹ is a heterocycloalkyl group, wherein the heterocycloalkyl group is selected from morpholinyl, piperidinyl, piperazinyl, pyrrolidinyl, imidazolidinyl, 3-azabicyclo[3.1.0]hexyl, 1,2,3,6-tetrahydropyridinyl, hexahydrocyclopenta[c]pyrrol-2(1H)-yl, hexahydrocyclopenta[c]pyrrol-5(1H)-yl, hexahydropyrrolo[3,4-c]pyrrol-2(1H)-yl), (4aR,8aR)-octahydro-1H-pyrido[3,4-b][1,4]oxazinyl, hexahydropyrrolo[3,4-b]pyrrol-2(1H)-yl), hexahydropyrrolo[3,4-b]pyrrol-5(1H)-yl), thiazolidinyl, tetrahydro-3aH[1,3]dioxolo[4,5-c]pyrrolyl, hexahydropyrrolo[1,2-a]pyrazin-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from halo, alkyl optionally substituted with 1-3 halo, carboxyalkyl, amino, hydroxyl, hydroxyalkyl, alkoxy optionally substituted with 1-3 halo, alkoxyalkyl optionally substituted with 1-3 halo, —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, (C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with NR⁸R⁹, —(CH₂)₀₋₃—N(R⁶)R⁷,
—C(O)—N(R⁸)R⁹, alkyl-C(O)N(H)(alkyl)-, aryl, arylalkyl optionally substituted with 1 or 2 groups sele2cted from halo and alkyl, alkoxycarbonyl and aminoalkyl, provided that the optionally substituted heterocycloalkyl group cannot be substituted with more than one group selected from —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with alkyl, (CH₂)₀₋₃—N(R⁸)R⁷, —C(O)—N(R⁸)R⁹, alkyl-C(O)N(H)(alkyl)-, aryl, arylalkyl optionally substituted with 1 or 2 groups selected from halo and alkyl, and alkoxycarbonyl; and
R⁸, R⁷, R⁸, R⁹, and R¹⁰ are as defined in claim 1.

7. The compound of Formula IA according to claim 2, or a pharmaceutically acceptable salt thereof, wherein:
R¹ is selected from NR⁴R⁵, —(C₁-C₄)alky-NR⁸R⁹, —O—(C₁-C₄)alky-NR⁸R⁹, triazolyl optionally substituted with phenylmethyl, 1H-pyrrolo[2,3-b]pyridinyl, 1H-indolyl, pyridinyl optionally substituted with amino or methoxy, pyrazolyl, pyrimidinyl optionally substituted with amino, (5-6 membered)heterocycloalkyl optionally substituted with hydroxyl, and amino;
R³ is phenyl, pyridine or pyrimidine, wherein the phenyl, pyridine or pyrimidine is optionally substituted with 1 or 2 substituents independently selected from amine, alkoxy, hydroxyl, alkyl and aminocarbonyl;
R⁴ is selected from H, methyl, ethyl and propyl;
R⁵ is selected from is selected from propyl, 1-methylethyl, methyl, ethyl, propyl, 2-methylpropyl, phenylethyl, dimethylaminoethyl, methoxymethyl, piperidinyl optionally substituted with methyl, cyclopentyl, dimethylaminocarbonylmethyl, ethylpyrrolidinylmethyl, piperidinylethyl, methylaminoethyl, aminoethyl, pyrrolidinylethyl, 2-propane-1,3 diol, phenyl, carboxymethyl, dimethylaminopropyl, dimethylaminopropan-2-yl, dimethylaminopropan-1-ol-2-yl, methylaminopropyl, aminocyclohexyl, methoxyethyl, (2R)-pyrrolidinylmethyl, (2S)-pyrrolidinylmethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino, (R)-benzyl 2-amino-3-hydroxypropylcarbamate and methoxymethyl;
or R⁴ and R⁵, together with the nitrogen to which they are both attached, join together to form a heteroaryl group or a heterocycloalkyl group, wherein the heteroaryl group is selected from (1) isoindolinyl, (2) 1,2,3,4-tetrahydroisoquinolinyl optionally substituted with hydroxyl, and (3) indolinyl optionally substituted with hydroxyalkyl, and wherein the heterocycloalkyl group is selected from morpholinyl, piperidinyl, piperazinyl, pyrrolidinyl, imidazolidinyl, 3-azabicyclo[3.1.0]hexyl, 1,2,3,6-tetrahydropyridinyl, hexahydrocyclopenta[c]pyrrol-2(1H)-yl, hexahydrocyclopenta[c]pyrrol-5(1H)-yl, hexahydropyrrolo[3,4-c]pyrrol-2(1H)-yl), (4aR,8aR)-octahydro-1H-pyrido[3,4-b][1,4]oxazinyl, hexahydropyrrolo[3,4-b]pyrrol-2(1H)-yl), hexahydropyrrolo[3,4-b]pyrrol-5(1H)-yl), thiazolidinyl, tetrahydro-3aH[1,3]dioxolo[4,5-c]pyrrolyl, hexahydropyrrolo[1,2-a]pyrazin-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from halo, alkyl optionally substituted with 1-3 halo, carboxyalkyl, amino, hydroxyl, hydroxyalkyl, alkoxy optionally substituted with 1-3 halo, alkoxyalkyl optionally substituted with 1-3 halo, —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, (C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with NR⁸R⁹, —(CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, alkoxycarbonyl and aminoalkyl, provided that the optionally substituted heterocycloalkyl group is substituted cannot be substituted with more than one group selected from —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂,
—(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with alkyl, (CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, and alkoxycarbonyl; and
R⁶, R⁷, R⁸, R⁹, and R¹⁰ are as defined in claim 1.

8. The compound of Formula IA according to claim 2, or a pharmaceutically acceptable salt thereof, wherein:
R¹ is a heterocycloalkyl group, wherein the heterocycloalkyl group is selected from morpholinyl, piperidinyl, piperazinyl, pyrrolidinyl, imidazolidinyl, 3-azabicyclo[3.1.0]hexyl, 1,2,3,6-tetrahydropyridinyl, hexahydrocyclopenta[c]pyrrol-2(1H)-yl, hexahydrocyclopenta[c]pyrrol-5(1H)-yl, hexahydropyrrolo[3,4-c]pyrrol-2(1H)-yl), (4aR,8aR)-octahydro-1H-pyrido[3,4-b][1,4]oxazinyl, hexahydropyrrolo[3,4-b]pyrrol-2(1H)-yl), hexahydropyrrolo[3,4-b]pyrrol-5(1H)-yl), thiazolidinyl, tetrahydro-3aH[1,3]dioxolo[4,5-c]pyrrolyl, hexahydropyrrolo[1,2-a]pyrazin-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from halo, alkyl optionally substituted with 1-3 halo, carboxyalkyl, amino, hydroxyl, hydroxyalkyl, alkoxy optionally substituted with 1-3 halo, alkoxyalkyl optionally substituted with 1-3 halo, —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, (C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with NR⁸R⁹, —(CH₂)₀₋₃—N(R⁶)R⁷,
—C(O)—N(R⁸)R⁹, alkyl-C(O)N(H)(alkyl)-, aryl, arylalkyl optionally substituted with 1 or 2 groups selected from halo and alkyl, alkoxycarbonyl and aminoalkyl, provided that the optionally substituted heterocycloalkyl group cannot be substituted with more than one group selected from —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with alkyl, (CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, and alkoxycarbonyl; and
R⁶, R⁷, R⁸, R⁹, and R¹⁰ are as defined in claim 1.

9. The compound according to claim 1, or a pharmaceutically acceptable salt thereof, having Formula II, III, IV, V, VI or VII: 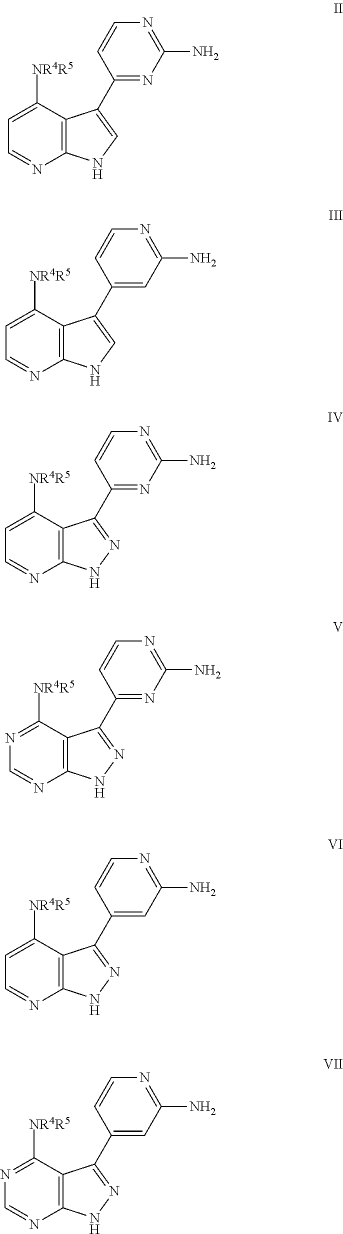
wherein R⁴ and R⁵ are as defined in claim 1.

10. The compound according to claim 9, or a pharmaceutically acceptable salt thereof, wherein:
R⁴ is selected from H, 1-methylethyl, methyl, ethyl, propyl, 2-methylpropyl, phenylethyl, dimethylaminoethyl, methoxymethyl, piperidinyl optionally substituted with methyl, cyclopentyl, dimethylaminocarbonylmethyl, ethylpyrrolidinylmethyl, piperidinylethyl, methylaminoethyl, aminoethyl, pyrrolidinylethyl, 2-propane-1,3 diol, phenyl, carboxymethyl, dimethylaminopropyl, dimethylaminopropan-2-yl, dimethylaminopropan-1-ol-2-yl, methylaminopropyl, aminocyclohexyl, methoxyethyl, (2R)-pyrrolidinylmethyl, (2S)-pyrrolidinylmethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino, (R)-benzyl 2-amino-3-hydroxypropylcarbamate and methoxymethyl;
R⁵ is selected from is selected from propyl, 1-methylethyl, methyl, ethyl, propyl, 2-methylpropyl, phenylethyl, dimethylaminoethyl, methoxymethyl, piperidinyl optionally substituted with methyl, cyclopentyl, dimethylaminocarbonylmethyl, ethylpyrrolidinylmethyl, piperidinylethyl, methylaminoethyl, aminoethyl, pyrrolidinylethyl, 2-propane-1,3 diol, phenyl, carboxymethyl, dimethylaminopropyl, dimethylaminopropan-2-yl, dimethylaminopropan-1-ol-2-yl, methylaminopropyl, aminocyclohexyl, methoxyethyl, (2R)-pyrrolidinylmethyl, (2S)-pyrrolidinylmethyl, dimethylaminoethyl, pyrrolidinyl optionally substituted with methyl, propane-1,3-diol, hydroxymethyl, hydroxyethyl, aminomethyl, dimethylaminomethylcarbonylaminoethyl, dimethylamino, (R)-benzyl 2-amino-3-hydroxypropylcarbamate and methoxymethyl;
or R⁴ and R⁵, together with the nitrogen to which they are both attached, join together to form a heteroaryl group or a heterocycloalkyl group, wherein the heteroaryl group is selected from (1) isoindolinyl, (2) 1,2,3,4-tetrahydroisoquinolinyl optionally substituted with hydroxyl, and (3) indolinyl optionally substituted with hydroxyalkyl, and wherein the heterocycloalkyl group is selected from morpholinyl, piperidinyl, piperazinyl, pyrrolidinyl, imidazolidinyl, 3-azabicyclo[3.1.0]hexyl, 1,2,3,6-tetrahydropyridinyl, hexahydrocyclopenta[c]pyrrol-2(1H)-yl, hexahydrocyclopenta[c]pyrrol-5(1H)-yl, hexahydropyrrolo[3,4-c]pyrrol-2(1H)-yl), (4aR,8aR)-octahydro-1H-pyrido[3,4-b][1,4]oxazinyl, hexahydropyrrolo[3,4-b]pyrrol-2(1H)-yl), hexahydropyrrolo[3,4-b]pyrrol-5(1H)-yl), thiazolidinyl, tetrahydro-3aH[1,3]dioxolo[4,5-c]pyrrolyl, hexahydropyrrolo[1,2-a]pyrazin-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from halo, alkyl optionally substituted with 1-3 halo, carboxyalkyl, amino, hydroxyl, hydroxyalkyl, alkoxy optionally substituted with 1-3 halo, alkoxyalkyl optionally substituted with 1-3 halo, —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, (C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with NR⁸R⁹, —(CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, alkoxycarbonyl and aminoalkyl, provided that the optionally substituted heterocycloalkyl group cannot be substituted with more than one group selected from —(CH₂)₀₋₃-heterocycloalkyl optionally substituted with alkyl, ═N(H)—OH,
—N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with alkyl, (CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, and alkoxycarbonyl; and
R⁶, R⁷, R⁸, R⁹, and R¹⁰ are as defined in claim 1.

11. The compound of Formula II, III, IV, V, VI or VII according to claim 9, or a pharmaceutically acceptable salt thereof, wherein:
R⁴ and R⁵, together with the nitrogen to which they are both attached, join together to form a heterocycloalkyl group, wherein the heterocycloalkyl group is selected from morpholinyl, piperidinyl, piperazinyl, pyrrolidinyl, imidazolidinyl, 3-azabicyclo[3.1.0]hexyl, 1,2,3,6-tetrahydropyridinyl, hexahydrocyclopenta[c]pyrrol-2(1H)-yl, hexahydrocyclopenta[c]pyrrol-5(1H)-yl, hexahydropyrrolo[3,4-c]pyrrol-2(1H)-yl), (4aR,8aR)-octahydro-1H-pyrido[3,4-b][1,4]oxazinyl, hexahydropyrrolo[3,4-b]pyrrol-2(1H)-yl), hexahydropyrrolo[3,4-b]pyrrol-5(1H)-yl), thiazolidinyl, tetrahydro-3aH[1,3]dioxolo[4,5-c]pyrrolyl, hexahydropyrrolo[1,2-a]pyrazin-2(1H)-yl), azepanyl, 1,4-diazepanyl, (1S,4S)-2,5-diazabicyclo[2.2.1]heptanyl and azetanyl, wherein the heterocycloalkyl group is optionally substituted with 1, 2 or 3 substituents independently selected from halo, alkyl optionally substituted with 1-3 halo, carboxyalkyl, amino, hydroxyl, hydroxyalkyl, alkoxy optionally substituted with 1-3 halo, alkoxyalkyl optionally substituted with 1-3 halo, —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, (C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with NR⁸R⁹, —(CH₂)₀₋₃—N(R⁶)R⁷,
—C(O)—N(R⁸)R⁹, alkyl-C(O)N(H)(alkyl)-, aryl, arylalkyl optionally substituted with 1 or 2 groups selected from halo and alkyl, alkoxycarbonyl and aminoalkyl, provided that the optionally substituted heterocycloalkyl group cannot be substituted with more than one group selected from —(CH₂)₀₋₃-heterocycloalkyl optionally substituted with alkyl, ═N(H)—OH, —N(Boc)alkyl, spiroheterocycloalkyl optionally substituted with —C(O)OR⁹ or —NH₂, —(C₁-C₃)alkyl-O—C(O)—C(H)(NH₂)—(C₁-C₃)alkyl, —(CH₂)₀₋₃—O—C(O)—NR⁸R⁹, —C(O)-aryl-R¹⁰, alkylcarbonylamino, alkoxyalkylcarbonylamino, heteroaryl optionally substituted with alkyl, (CH₂)₀₋₃—N(R⁶)R⁷, —C(O)—N(R⁸)R⁹, alkyl-C(O)N(H)(alkyl)-, aryl, arylalkyl optionally substituted with 1 or 2 groups selected from halo and alkyl, and alkoxycarbonyl; and
R⁶, R⁷, R⁸, R⁹, and R¹⁰ are as defined in claim 1.

12. The compound of Formula II, III, IV, V, VI or VII according to claim 9, or a pharmaceutically acceptable salt thereof, wherein:
R⁴ is H; and
R⁵ is selected from —(C₁-C₃)alkylamino optionally substituted with 1-2 substituents selected from halo, hydroxymethyl and hydroxyl, amino(C₁-C₃)alkylamino, pyrrolidinyl(C₁-C₄)alkylamino, pyridinyll (C₁-C₃)alkylamino, —(C₁-C₃)alkylamino(C₁-C₃)alkylamino, —(C₁-C₃)dialkylamino(C₁-C₃)alkylamino optionally substituted with hydroxymethyl, or
R⁴ and R⁵, together with the nitrogen atom to which they are attached, join together to form piperidinyl, morpholinyl and pyrrolidinyl, wherein the piperidinyl, morpholinyl and pyrrolidinyl are optionally substituted with 1, 2 or 3 groups selected from —(C₁-C₃)alkyl optionally substituted with 1-3 substituents selected from halo and hydroxyl, hydroxyl, hydroxyalkyl, alkylamino, methoxy, aminocarbonyl, amine, aminoalkyl, hydroxymethyl, aminomethyl, dimethylamino, dimethylaminoethoxy, and methoxymethyl.

13. The compound of Formula IB according to claim 2, or a pharmaceutically acceptable salt thereof, wherein
R^{Xa} is pyridinyl, 1H-isoindole, halo, phenyl optionally substituted with amine, hydroxyalkyl, aminocarbony, dialkylaminocarbonyl, —S(O)₂—NH₂, and alkylcarbonylamino;
R^{Xb} is selected from (1) pyridine optionally substituted with halo or amine, (2) pyrimidiny optionally substituted with amine or dialkylaminoalkylcarbonylaminoalkylamino, (3) phenyl optionally substituted with —S(O)₂—N(H)—(C₁-C₃)alkyl-OH, —S(O)₂—N(H)—(C₁-C₃)alkyl-phenyl, or hydroxyallyl, (4) 1H-pyrazolo[3,4-b]pyridine, imidazolyl, —S(O)₂—N(H)—(C₁-C₃)alkyl-OH,- and imidazolyl.

14. The compound of Formula I according to claim 1, selected from one of the following compounds, or a pharmaceutically acceptable salt thereof:
4-[4-(4-ethylpiperazin-1-yl)-1H-pyrrolo[2,3-b]pyridin-3-yl]pyrimidin-2-amine;
3-(2-aminopyrimidin-4-yl)-N,N-diethyl-1H-pyrrolo[2,3-b]pyridin-4-amine;
3-(2-aminopyrimidin-4-yl)-N-(2-methylpropyl)-1H-pyrrolo[2,3-b]pyridin-4-amine;
4-[4-(hexahydrocyclopenta[c]pyrrol-2(1H)-yl)-1H-pyrrolo[2,3-b]pyridin-3-yl]pyrimidin-2-amine;
4-(methyloxy)-6-(4-piperidin-1-yl-1H-pyrrolo[2,3-b]pyridin-3-yl)pyrimidin-2-amine;
3-(2-aminopyrimidin-4-yl)-N-ethyl-1H-pyrrolo[2,3-b]pyridin-4-amine;
3-(2-aminopyrimidin-4-yl)-N-methyl-N-(1-methylpyrrolidin-3-yl)-1H-pyrrolo[2,3-b]pyridin-4-amine;
2-{1-[3-(2-aminopyrimidin-4-yl)-1H-pyrrolo[2,3-b]pyridin-4-yl]piperidin-3-yl}ethanol;
N-[3-(2-aminopyrimidin-4-yl)-1H-pyrrolo[2,3-b]pyridin-4-yl]-N,N′,N′-trimethylethane-1,2-diamine;
4-{4-[2-(aminomethyl)pyrrolidin-1-yl]-1H-pyrrolo[2,3-b]pyridin-3-yl}pyrimidin-2-amine;
{1-[3-(2-aminopyrimidin-4-yl)-1H-pyrrolo[2,3-b]pyridin-4-yl]pyrrolidin-2-yl}methanol;
4-(4-pyrrolidin-1-yl-1H-pyrrolo[2,3-b]pyridin-3-yl)pyrimidin-2-amine;
4-[4-(1-methylhexahydropyrrolo[3,4-b]pyrrol-5(1H)-yl)-1H-pyrrolo[2,3-b]pyridin-3-yl]pyrimidin-2-amine;
4-[4-(4-methylpiperazin-1-yl)-1H-pyrrolo[2,3-b]pyridin-3-yl]pyridin-2-amine;
4-{4-[3-(methylamino)pyrrolidin-1-yl]-1H-pyrrolo[2,3-b]pyridin-3-yl}pyrimidin-2-amine;
4-[4-(4-methylpiperazin-1-yl)-1H-pyrrolo[2,3-b]pyridin-3-yl]pyrimidin-2-amine;
ethyl 4-[3-(2-aminopyrimidin-4-yl)-1H-pyrrolo[2,3-b]pyridin-4-yl]piperazine-1-carboxylate;
4-(4-morpholin-4-yl-1H-pyrrolo[2,3-b]pyridin-3-yl)pyrimidin-2-amine;
4-{4-[(3S)-3-aminopyrrolidin-1-yl]-1H-pyrrolo[2,3-b]pyridin-3-yl}pyrimidin-2-amine;
4-[4-(5-methylhexahydropyrrolo[3,4-c]pyrrol-2(1H)-yl)-1H-pyrrolo[2,3-b]pyridin-3-yl]pyrimidin-2-amine;
4-[4-(4-methylpiperazin-1-yl)-1H-pyrrolo[2,3-b]pyridin-3-yl]pyridin-2-ol;
4-[4-(4-phenylpiperazin-1-yl)-1H-pyrrolo[2,3-b]pyridin-3-yl]pyrimidin-2-amine;
3-(2-aminopyrimidin-4-yl)-N-methyl-N-(1-methylethyl)-1H-pyrrolo[2,3-b]pyridin-4-amine;
3-(2-aminopyrimidin-4-yl)-N,N-dimethyl-1H-pyrrolo[2,3-b]pyridin-4-amine;
4-(5-phenyl-1H-pyrrolo[2,3-b]pyridin-3-yl)pyrimidin-2-amine;
4-[4-(3-aminopyrrolidin-1-yl)-1H-pyrrolo[2,3-b]pyridin-3-yl]pyrimidin-2-amine;
4-[4-(4-methylpiperidin-1-yl)-1H-pyrrolo[2,3-b]pyridin-3-yl]pyrimidin-2-amine;
4-[4-(3,5-dimethylpiperidin-1-yl)-1H-pyrrolo[2,3-b]pyridin-3-yl]pyrimidin-2-amine;
3-(2-aminopyrimidin-4-yl)-1H-pyrrolo[2,3-b]pyridin-5-amine;
4-(4-piperidin-1-yl-1H-pyrrolo[2,3-b]pyridin-3-yl)pyrimidin-2-amine;
N-{1-[3-(2-aminopyrimidin-4-yl)-1H-pyrrolo[2,3-b]pyridin-4-yl]pyrrolidin-3-yl}-N-methylacetamide;
4-methyl-6-[4-(4-methylpiperazin-1-yl)-1H-pyrrolo[2,3-b]pyridin-3-yl]pyrimidi2n-2-amine;
3-(2-aminopyrimidin-4-yl)-N-methyl-N-[2-(methyloxy)ethyl]-1H-pyrrolo[2,3-b]pyridin-4-amine;
4-{4-[4-(dimethylamino)piperidin-1-yl]-1H-pyrrolo[2,3-b]pyridin-3-yl}pyrimidin-2-amine;
4-[4-(4-aminopiperidin-1-yl)-1H-pyrrolo[2,3-b]pyridin-3-yl]pyrimidin-2-amine;
4-[4-(3,4,5-trimethylpiperazin-1-yl)-1H-pyrrolo[2,3-b]pyridin-3-yl]pyrimidin-2-amine;
6-(4-piperidin-1-yl-1H-pyrrolo[2,3-b]pyridin-3-yl)pyrimidine-2,4-diamine;
N-{1-[3-(2-aminopyrimidin-4-yl)-1H-pyrrolo[2,3-b]pyridin-4-yl]pyrrolidin-3-yl}acetamide;
4-(4-{(2R)-2-[(methyloxy)methyl]pyrrolidin-1-yl}-1H-pyrrolo[2,3-b]pyridin-3-yl)pyrimidin-2-amine;
3-(4-(piperidin-1-yl)-1H-pyrrolo[2,3-b]pyridin-3-yl)benzamide;
4-[4-(3-aminopiperidin-1-yl)-1H-pyrrolo[2,3-b]pyridin-3-yl]pyrimidin-2-amine;
3-2(2-aminopyrimidin-4-yl)-N-methyl-N-(1-methylpiperidin-4-yl)-1H-pyrrolo[2,3-b]pyridin-4-amine;
3-(2-aminopyrimidin-4-yl)-N-methyl-N-(2-phenylethyl)-1H-pyrrolo[2,3-b]pyridin-4-amine;
(R)-(1-(3-(2-aminopyrimidin-4-yl)-1H-pyrrolo[2,3-b]pyridin-4-yl)pyrrolidin-2-yl)methanol;
4-[4-(3,5-dimethylpiperazin-1-yl)-1H-pyrrolo[2,3-b]pyridin-3-yl]pyrimidin-2-amine;
(3R)-1-[3-(2-aminopyrimidin-4-yl)-1H-pyrrolo[2,3-b]pyridin-4-yl]pyrrolidin-3-ol;
4-{4-[3-(dimethylamino)pyrrolidin-1-yl]-1H-pyrrolo[2,3-b]pyridin-3-yl}pyrimidin-2-amine;
(3S)-1-[3-(2-aminopyrimidin-4-yl)-1H-pyrrolo[2,3-b]pyridin-4-yl]pyrrolidin-3-ol;
4-(4-azepan-1-yl-1H-pyrrolo[2,3-b]pyridin-3-yl)pyrimidin-2-amine;
3-(2-aminopyrimidin-4-yl)-N,N-dipropyl-1H-pyrrolo[2,3-b]pyridin-4-amine;
4-{4-[3-(methyloxy)pyrrolidin-1-yl]-1H-pyrrolo[2,3-b]pyridin-3-yl}pyrimidin-2-amine;
4-{4-[(3R)-3-aminopyrrolidin-1-yl]-1H-pyrrolo[2,3-b]pyridin-3-yl}pyrimidin-2-amine;
4-(4-((1S,4S)-5-methyl-2,5-diazabicyclo[2.2.1]heptan-2-yl)-1H-pyrrolo[2,3-b]pyridin-3-yl)pyrimidin-2-amine;
N-methyl-4-(4-piperidin-1-yl-1H-pyrrolo[2,3-b]pyridin-3-yl)pyrimidin-2-amine;
5-(2-aminopyrimidin-4-yl)-N,N-dimethyl-7H-pyrrolo[2,3-d]pyrimidin-4-amine;
4-(4-(hexahydropyrrolo[1,2-a]pyrazin-2(1H)-yl)-1H-pyrrolo[2,3-b]pyridin-3-yl)pyrimidin-2-amine;
4-(4-{(2S)-2-[(methyloxy)methyl]pyrrolidin-1-yl}-1H-pyrrolo[2,3-b]pyridin-3-yl)pyrimidin-2-amine;
4-[4-(3,4-dimethylpiperazin-1-yl)-1H-pyrrolo[2,3-b]pyridin-3-yl]pyrimidin-2-amine;
4-(4-piperazin-1-yl-1H-pyrazolo[3,4-d]pyrimidin-3-yl)pyridin-2-amine;
{(2S)-1-[3-(2-aminopyrimidin-4-yl)-1H-pyrrolo[2,3-b]pyridin-4-yl]pyrrolidin-2-yl}methanol;
4-[4-(dimethylamino)-1H-pyrazolo[3,4-b]pyridin-3-yl]pyridin-2-ol;
4-[4-(dimethylamino)-1H-pyrazolo[3,4-d]pyrimidin-3-yl]phenol;
4-[4-(hexahydropyrrolo[3,4-b]pyrrol-1(2H)-yl)-1H-pyrrolo[2,3-b]pyridin-3-yl]pyrimidin-2-amine;
4-[4-(4-methyl-1,4-diazepan-1-yl)-1H-pyrrolo[2,3-b]pyridin-3-yl]pyrimidin-2-amine;
3-(2-aminopyrimidin-4-yl)-N,N-dimethyl-1H-pyrazolo[3,4-b]pyridin-4-amine;
4-[4-(3,3-dimethylpyrrolidin-1-yl)-1H-pyrrolo[2,3-b]pyridin-3-yl]pyrimidin-2-amine;
4-{4-[(3S)-3-fluoropyrrolidin-1-yl]-1H-pyrrolo[2,3-b]pyridin-3-yl}pyrimidin-2-amine;
1-[3-(2-aminopyrimidin-4-yl)-1H-pyrrolo[2,3-b]pyridin-4-yl]piperidin-4-ol;
N′-[3-(2-aminopyrimidin-4-yl)-1H-pyrrolo[2,3-b]pyridin-4-yl]-N,N-dimethylethane-1,2-diamine;
3-(2-aminopyridin-4-yl)-N,N-dimethyl-1H-pyrazolo[3,4-d]pyrimidin-4-amine;
3-(2-aminopyridin-4-yl)-N,N-dimethyl-1H-pyrazolo[3,4-b]pyridin-4-amine;
3-(2-aminopyrimidin-4-yl)-1H-pyrazolo[3,4-d]pyrimidin-4-amine;
1-[3-(2-aminopyrimidin-4-yl)-1H-pyrrolo[2,3-b]pyridin-4-yl]piperidin-3-ol;
2-({(3S)-1-[3-(2-aminopyrimidin-4-yl)-1H-pyrrolo[2,3-b]pyridin-4-yl]pyrrolidin-3-yl}amino)ethanol;
{1-[3-(2-aminopyrimidin-4-yl)-1H-pyrrolo[2,3-b]pyridin-4-yl]pyrrolidin-3-yl}methanol;
3-(2-aminopyrimidin-4-yl)-N-cyclopentyl-1H-pyrrolo[2,3-b]pyridin-4-amine;
4-{4-[(3S,4R)-3-amino-4-phenylpyrrolidin-1-yl]-1H-pyrrolo[2,3-b]pyridin-3-yl}pyrimidin-2-amine;
N-({1-[3-(2-aminopyrimidin-4-yl)-1H-pyrrolo[2,3-b]pyridin-4-yl]pyrrolidin-2-yl}methyl)-N²,N²-dimethylglycinamide;
4-(4-{4-[2-(methyloxy)ethyl]piperazin-1-yl}-1H-pyrrolo[2,3-b]pyridin-3-yl)pyrimidin-2-amine;
4-{4-[(3S)-3-(ethylamino)pyrrolidin-1-yl}-1H-pyrrolo[2,3-b]pyridin-3-yl]pyrimidin-2-amine;
3-(2-aminopyrimidin-4-yl)-N-pyrrolidin-3-yl-1H-pyrrolo[2,3-b]pyridin-4-amine;
4-{4-[3-(aminomethyl)pyrrolidin-1-yl]-1H-pyrrolo[2,3-b]pyridin-3-yl}pyrimidin-2-amine;
2-{4-[3-(2-aminopyrimidin-4-yl)-1H-pyrrolo[2,3-b]pyridine-4-yl]piperazin-1-yl}ethanol;
4-[4-(2,7-diazaspiro[4.4]non-2-yl)-1H-pyrrolo[2,3-b]pyridin-3-yl]pyrimidin-2-amine;
3-(2-aminopyrimidin-4-yl)-N-(1,2,2-trimethylpropyl)-1H-pyrrolo[2,3-b]pyridin-4-amine;
3-{[3-(2-aminopyrimidin-4-yl)-1H-pyrrolo[2,3-b]pyridine-4-yl]amino}propane-1,2-diol;
(3R,4R)-4-amino-1-[3-(2-aminopyrimidin-4-yl)-1H-pyrrolo[2,3-b]pyridin-4-yl]pyrrolidin-3-ol;
3-(2-aminopyrimidin-4-yl)-N-piperidin-3-yl-1H-pyrrolo[2,3-b]pyridin-4-amine;
(2S)-3-({(3S)-1-[3-(2-aminopyrimidin-4-yl)-1H-pyrrolo[2,3-b]pyridin-4-yl]pyrrolidin-3-yl}amino)propane-1,2-diol;
4-{4-[4-(2-aminoethyl)piperazin-1-yl]-1H-pyrrolo[2,3-b]pyridin-3-yl}pyrimidin-2-amine;
4-{4-[4-(1-methylethyl)piperazin-1-yl]-1H-pyrrolo[2,3-b]pyridin-3-yl}pyrimidin-2-amine;
4-[4-(5-methylhexahydropyrrolo[3,4-b]pyrrol-1(2H)-yl)-1H-pyrrolo[2,3-b]pyridin-3-yl]pyrimidin-2-amine;
2-{(2S)-1-[3-(2-aminopyrimidin-4-yl)-1H-pyrrolo[2,3-b]pyridin-4-yl]pyrrolidin-2-yl}propan-2-ol;
4-{4-[(3S)-3-(methyloxy)pyrrolidin-1-yl]-1H-pyrrolo[2,3-b]pyridin-3-yl}pyrimidin-2-amine;
4-{4-[(3R)-3-fluoropyrrolidin-1-yl]-1H-pyrrolo[2,3-b]pyridin-3-yl}pyrimidin-2-amine;
1-[3-(2-aminopyrimidin-4-yl)-1H-pyrrolo[2,3-b]pyridin-4-yl]-L-prolinamide;
3-(2-aminopyrimidin-4-yl)-N-phenyl-1H-pyrrolo[2,3-b]pyridin-4-amine;
4-{4-[(3R)-3-(methyloxy)pyrrolidin-1-yl]-1H-pyrrolo[2,3-b]pyridin-3-yl}pyrimidin-2-amine;
3-(2-aminopyrimidin-4-yl)-N-(1-methylpyrrolidin-3-yl)-1H-pyrrolo[2,3-b]pyridin-4-amine;
3-(2-aminopyrimidin-4-yl)-N-methyl-N-phenyl-1H-pyrrolo[2,3-b]pyridin-4-amine;
{(3S)-1-[3-(2-aminopyrimidin-4-yl)-1H-pyrrolo[2,3-b]pyridin-4-yl]pyrrolidin-3-yl}methanol;
{4-[3-(2-aminopyrimidin-4-yl)-1H-pyrrolo[2,3-b]pyridin-4-yl]-1-methylpiperazin-2-yl}methanol;
N-[3-(2-aminopyrimidin-4-yl)-1H-pyrrolo[2,3-b]pyridin-4-yl]-N-methylglycine;
{(3R)-1-[3-(2-aminopyrimidin-4-yl)-1H-pyrrolo[2,3-b]pyridin-4-yl]pyrrolidin-3-yl}methanol;
(3R,5S)-1-[3-(2-aminopyrimidin-4-yl)-1H-pyrrolo[2,3-b]pyridin-4-yl]-5-(hydroxymethyl)pyrrolidin-3-ol;
(4S)-1-[3-(2-aminopyrimidin-4-yl)-1H-pyrrolo[2,3-b]pyridin-4-yl]-4-hydroxy-L-prolinamide;
4-(4-{(3S)-3-[(3,3,3-trifluoropropyl)amino]pyrrolidin-1-yl}-1H-pyrrolo[2,3-b]pyridin-3-yl)pyrimidin-2-amine;
{3-amino-1-[3-(2-aminopyrimidin-4-yl)-1H-pyrrolo[2,3-b]pyridin-4-yl]pyrrolidin-3-yl}methanol;
4-{4-[(2S)-2-(fluoromethyl)pyrrolidin-1-yl]-1H-pyrrolo[2,3-b]pyridin-3-yl}pyrimidin-2-amine;
(3S,4S)-1-[3-(2-aminopyrimidin-4-yl)-1H-pyrrolo[2,3-b]pyridin-4-yl]pyrrolidine-3,4-diol;
1-[3-(2-aminopyrimidin-4-yl)-1H-pyrrolo[2,3-b]pyridin-4-yl]azetidin-3-ol;
(3S)-1-[3-(2-aminopyrimidin-4-yl)-1H-pyrrolo[2,3-b]pyridin-4-yl]piperidin-3-ol;
2-{(2S)-1-[3-(2-aminopyrimidin-4-yl)-1H-pyrrolo[2,3-b]pyridin-4-yl]pyrrolidin-2-yl}acetamide;
(3S,5S)-1-[3-(2-aminopyrimidin-4-yl)-1H-pyrrolo[2,3-b]pyridin-4-yl]-5-(hydroxymethyl)pyrrolidin-3-ol;
1-[3-(2-aminopyrimidin-4-yl)-1H-pyrrolo[2,3-b]pyridin-4-yl]-N-methyl-L-prolinamide;
(3R)-1-[3-(2-aminopyrimidin-4-yl)-1H-pyrrolo[2,3-b]pyridin-4-yl]pyrrolidine-3-carboxylic acid;
(3R)-1-[3-(2-aminopyrimidin-4-yl)-1H-pyrrolo[2,3-b]pyridin-4-yl]pyrrolidine-3-carboxamide;
{1-[3-(2-aminopyrimidin-4-yl)-1H-pyrrolo[2,3-b]pyridin-4-yl]piperidin-2-yl}methanol;
2-{(2S)-1-[3-(2-aminopyrimidin-4-yl)-1H-pyrrolo[2,3-b]pyridin-4-yl]pyrrolidin-2-yl}ethanol;
(3R,4S)-1-[3-(2-aminopyrimidin-4-yl)-1H-pyrrolo[2,3-b]pyridin-4-yl]pyrrolidine-3,4-diol;
(2S)-1-[3-(2-aminopyrimidin-4-yl)-1H-pyrrolo[2,3-b]pyridin-4-yl]piperidine-2-carboxamide;
(S)-{1-[3-(2-aminopyrimidin-4-yl)-1H-pyrrolo[2,3-b]pyridin-4-yl]piperidin-4-yl}(4-chlorophenyl)methanol;
ethyl (3S)-1-[3-(2-aminopyrimidin-4-yl)-1H-pyrrolo[2,3-b]pyridin-4-yl]pyrrolidine-3-carboxylate;
(3S)-1-[43-(2-aminopyrimidin-4-yl)-1H-pyrrolo[2,3-b]pyridin-4-yl]-N-methylpyrrolidine-3-carboxamide;
4-{4-[(2R,6S)-2,6-dimethylmorpholin-4-yl]-1H-pyrrolo[2,3-b]pyridin-3-yl}pyrimidin-2-amine;
4-[4-(3-morpholin-4-ylpyrrolidin-1-yl)-1H-pyrrolo[2,3-b]pyridin-3-yl]pyrimidin-2-amine;
(3R)-1-[3-(2-aminopyrimidin-4-yl)-1H-pyrrolo[2,3-b]pyridin-4-yl]piperidin-3-ol;
(4S)-1-[3-(2-aminopyrimidin-4-yl)-1H-pyrrolo[2,3-b]pyridin-4-yl]-4-fluoro-L-prolinamide;
{(2S,4S)-1-[3-(2-aminopyrimidin-4-yl)-1H-pyrrolo[2,3-b]pyridin-4-yl]-4-fluoropyrrolidin-2-yl}methanol;
4-{4-[(3R,4R)-3-(methylamino)-4-(methyloxy)pyrrolidin-1-yl]-1H-pyrrolo[2,3-b]pyridin-3-yl}pyrimidin-2-amine;
3-(2-aminopyrimidin-4-yl)-N-(1,4-dioxan-2-ylmethyl)-1H-pyrrolo[2,3-b]pyridin-4-amine;
(1R)-1-{(2S)-1-[3-(2-aminopyrimidin-4-yl)-1H-pyrrolo[2,3-b]pyridin-4-yl]pyrrolidin-2-yl}ethanol;
(3R,4R)-1-[3-(2-aminopyrimidin-4-yl)-1H-pyrrolo[2,3-b]pyridin-4-yl]pyrrolidine-3,4-diol;
4-[4-(1,3-dihydro-2H-isoindol-2-yl)-1H-pyrrolo[2,3-b]pyridin-3-yl]pyrimidin-2-amine;
(3S)-1-[3-(2-aminopyrimidin-4-yl)-1H-pyrrolo[2,3-b]pyridin-4-yl]pyrrolidine-3-carboxamide;
4-{4-[(3R)-3-phenylpyrrolidin-1-yl]-1H-pyrrolo[2,3-b]pyridin-3-yl}pyrimidin-2-amine;
1-[3-(2-aminopyrimidin-4-yl)-1H-pyrrolo[2,3-b]pyridin-4-yl]-3-phenylpyrrolidin-3-ol;
4-{4-[3-(aminomethyl)azetidin-1-yl]-1H-pyrrolo[2,3-b]pyridin-3-yl}pyrimidin-2-amine;
(3R,4S)-4-(aminomethyl)-1-[3-(2-aminopyrimidin-4-yl)-1H-pyrrolo[2,3-b]pyridin-4-yl]pyrrolidin-3-ol;
(4R)-3-[3-(2-aminopyrimidin-4-yl)-1H-pyrrolo[2,3-b]pyridin-4-yl]-1,3-thiazolidine-4-carboxamide;
{(2S,4R)-1-[3-(2-aminopyrimidin-4-yl)-1H-pyrrolo[2,3-b]pyridin-4-yl]-4-fluoropyrrolidin-2-yl}methanol;
4-[3-(2-aminopyrimidin-4-yl)-1H-pyrrolo[2,3-b]pyridin-4-yl]-1-methylpiperazin-2-one;
methyl 7-[3-(2-aminopyrimidin-4-yl)-1H-pyrrolo[2,3-b]pyridin-4-yl]-2,7-diazaspiro[4.4]nonane-2-carboxylate;
1-[3-(2-aminopyrimidin-4-yl)-1H-pyrrolo[2,3-b]pyridin-4-yl]-3-methylpyrrolidin-3-ol;
4-[4-(3-aminoprop-1-yn-1-yl)-1H-pyrrolo[2,3-b]pyridin-3-yl]pyrimidin-2-amine;
4-{4-[3-(dimethylamino)prop-1-yn-1-yl]-1H-pyrrolo[2,3-b]pyridin-3-yl}pyrimidin-2-amine;
(2R,3S)-1-[3-(2-aminopyrimidin-4-yl)-1H-pyrrolo[2,3-b]pyridin-4-yl]-2-(hydroxymethyl)pyrrolidin-3-ol;
(3R,4R)-1-[3-(2-aminopyrimidin-4-yl)-1H-pyrrolo[2,3-b]pyridin-4-yl]-4-(methyloxy)pyrrolidin-3-ol;
4-[4-(3,3-difluoropyrrolidin-1-yl)-1H-pyrrolo[2,3-b]pyridin-3-yl]pyrimidin-2-amine;
4-{4-[3-(dimethylamino)propyl]-1H-pyrrolo[2,3-b]pyridin-3-yl}pyrimidin-2-amine;
4-[4-(3,4-dihydroisoquinolin-2(1H)-yl)-1H-pyrrolo[2,3-b]pyridin-3-yl]pyrimidin-2-amine;
4-[4-(2,3-dihydro-1H-indol-1-yl)-1H-pyrrolo[2,3-b]pyridin-3-yl]pyrimidin-2-amine;
3-(2-aminopyrimidin-4-yl)-N-(morpholin-2-ylmethyl)-1H-pyrrolo[2,3-b]pyridin-4-amine;
(3S,4S)-1-[3-(2-aminopyrimidin-4-yl)-1H-pyrrolo[2,3-b]pyridin-4-yl]-4-fluoropyrrolidin-3-ol;
(3R,4R)-1-[3-(2-aminopyrimidin-4-yl)-1H-pyrrolo[2,3-b]pyridin-4-yl]-4-hydroxypyrrolidine-3-carbonitrile;
[(2S,4S)-1-[3-(2-aminopyrimidin-4-yl)-1H-pyrrolo[2,3-b]pyridin-4-yl]-4-(methyloxy)pyrrolidin-2-yl]methanol;
{(2S)-1-[3-(2-aminopyrimidin-4-yl)-1H-pyrrolo[2,3-b]pyridin-4-yl]-4,4-difluoropyrrolidin-2-yl}methanol;
4-{4-[(3R,4R)-3-fluoro-4-(methyloxy)pyrrolidin-1-yl]-1H-pyrrolo[2,3-b]pyridin-3-yl}pyrimidin-2-amine;
2-{[3-(2-aminopyrimidin-4-yl)-1H-pyrrolo[2,3-b]pyridin-4-yl]amino}ethanol;
7-[3-(2-aminopyrimidin-4-yl)-1H-pyrrolo[2,3-b]pyridin-4-yl]-2,7-diazaspiro[4.4]nonane-2-carboxamide;
4-{4-[3-(butyloxy)-3-methylpiperidin-1-yl]-1H-pyrrolo[2,3-b]pyridin-3-yl}pyrimidin-2-amine;
(3S,5S)-1-[3-(2-aminopyrimidin-4-yl)-1H-pyrrolo[2,3-b]pyridin-4-yl]-5-(hydroxymethyl)-3-(trifluoromethyl)pyrrolidin-3-ol;
[(2S,4S)-1-[3-(2-aminopyrimidin-4-yl)-1H-pyrrolo[2,3-b]pyridin-4-yl]-4-(methyloxy)-4-(trifluoromethyl)pyrrolidin-2-yl]methanol;
(3S,4S)-4-amino-1-[3-(2-aminopyrimidin-4-yl)-1H-pyrrolo[2,3-b]pyridin-4-yl]piperidin-3-ol;
4-{4-[(5aR,8aR)-5a,6,8,8a-tetrahydro-4H,7H-pyrrolo[3,4-b][1,2,3]triazolo[1,5-d][1,4]oxazin-7-yl]-1H-pyrrolo[2,3-b]pyridin-3-yl}pyrimidin-2-amine;
4-{4-[1-(phenylmethyl)-1H-1,2,3-triazol-4-yl]-1H-pyrrolo[2,3-b]pyridin-3-yl}pyrimidin-2-amine;
{(2S)-1-[3-(2-aminopyrimidin-4-yl)-5-methyl-1H-pyrrolo[2,3-b]pyridin-4-yl]pyrrolidin-2-yl}methanol;
[(2R,3S)-1-[3-(2-aminopyrimidin-4-yl)-1H-pyrrolo[2,3-b]pyridin-4-yl]-3-(methyloxy)pyrrolidin-2-yl]methanol;
3-[3-(2-aminopyrimidin-4-yl)-1H-pyrrolo[2,3-b]pyridin-4-yl]prop-2-yn-1-ol;
1-[3-(2-aminopyrimidin-4-yl)-1H-pyrrolo[2,3-b]pyridin-4-yl]-1,2,3,6-tetrahydropyridin-3-ol;
2-{1-[3-(2-aminopyrimidin-4-yl)-1H-pyrrolo[2,3-b]pyridin-4-yl]piperidin-4-yl}propan-2-ol;
3-[3-(2-aminopyrimidin-4-yl)-1H-pyrrolo[2,3-b]pyridin-4-yl]propan-1-ol;
4-{4-[3-(methylamino)prop-1-yn-1-yl]-1H-pyrrolo[2,3-b]pyridin-3-yl}pyrimidin-2-amine;
{(2S)-1-[3-(2-aminopyrimidin-4-yl)-1H-pyrrolo[2,3-b]pyridin-4-yl]pyrrolidin-2-yl}methyl L-valinate;
1,1-dimethylethyl N-[3-(2-aminopyrimidin-4-yl)-1H-pyrrolo[2,3-b]pyridin-4-yl]glycinate;
{(2R,3S)-1-[3-(2-aminopyrimidin-4-yl)-1H-pyrrolo[2,3-b]pyridin-4-yl]-3-fluoropyrrolidin-2-yl}methanol;
{(3aR,4R,6aS)-5-[3-(2-aminopyrimidin-4-yl)-1H-pyrrolo[2,3-b]pyridin-4-yl]-2,2-dimethyltetrahydro-3aH-[1,3]dioxolo[4,5-c]pyrrol-4-yl}methanol;
4-{4-[(3R,4S)-3,4-bis(methyloxy)pyrrolidin-1-yl]-1H-pyrrolo[2,3-b]pyridin-3-yl}pyrimidin-2-amine;
3-(2-aminopyrimidin-4-yl)-N-[2-(methyloxy)ethyl]-1H-pyrrolo[2,3-b]pyridin-4-amine;
4-{4-[(3S,4S)-3,4-difluoropyrrolidin-1-yl]-1H-pyrrolo[2,3-b]pyridin-3-yl}pyrimidin-2-amine;
{(2S)-1-[3-(2-aminopyrimidin-4-yl)-1H-pyrrolo[2,3-b]pyridin-4-yl]pyrrolidin-2-yl}methyl carbamate;
{3-[3-(2-aminopyrimidin-4-yl)-1H-pyrrolo[2,3-b]pyridin-4-yl]phenyl}methanol;
4-(4-{[2-(dimethylamino)ethyl]oxy}-1H-pyrrolo[2,3-b]pyridin-3-yl)pyrimidin-2-amine;
4-[4-(3-azabicyclo[3.1.0]hex-3-yl)-1H-pyrrolo[2,3-b]pyridin-3-yl]pyrimidin-2-amine;
1-[3-(2-aminopyrimidin-4-yl)-1H-pyrrolo[2,3-b]pyridin-4-yl]imidazolidin-2-one;
{1-[3-(2-aminopyrimidin-4-yl)-1H-pyrrolo[2,3-b]pyridin-4-yl]piperidin-4-yl}(4-chlorophenyl)methanone;
4-{4-[3-(3-methyl-1,2,4-oxadiazol-5-yl)pyrrolidin-1-yl]-1H-pyrrolo[2,3-b]pyridin-3-yl}pyrimidin-2-amine;
4-[4-(3-phenylpyrrolidin-1-yl)-1H-pyrrolo[2,3-b]pyridin-3-yl]pyrimidin-2-amine;
4-{4-[(3R)-3-(ethyloxy)pyrrolidin-1-yl]-1H-pyrrolo[2,3-b]pyridin-3-yl}pyrimidin-2-amine;
3-{[3-(2-aminopyrimidin-4-yl)-1H-pyrrolo[2,3-b]pyridin-4-yl]oxy}propan-1-ol;
2-{[3-(2-aminopyrimidin-4-yl)-1H-pyrrolo[2,3-b]pyridin-4-yl]oxy}ethanol;
4-(4-{[2-(methyloxy)ethyl]oxy}-1H-pyrrolo[2,3-b]pyridin-3-yl)pyrimidin-2-amine
4-(1H-pyrrolo[2,3-b]pyridin-3-yl)pyrimidin-2-amine
N²-[3-(2-aminopyrimidin-4-yl)-1H-pyrrolo[2,3-b]pyridin-4-yl]-N,N-dimethylglycinamide;
(3aR,6aS)-5-[3-(2-aminopyrimidin-4-yl)-1H-pyrrolo[2,3-b]pyridin-4-yl]tetrahydro-3aH[1,3]dioxolo[4,5-c]pyrrol-2-one;
N-{(3R,4R)-1-[3-(2-aminopyrimidin-4-yl)-1H-pyrrolo[2,3-b]pyridin-4-yl]-4-hydroxypyrrolidin-3-yl}-2-(ethyloxy)acetamide;
N-{(3R,4R)-1-[3-(2-aminopyrimidin-4-yl)-1H-pyrrolo[2,3-b]pyridin-4-yl]-4-hydroxypyrrolidin-3-yl}acetamide;
{(2S)-1-[3-(2-aminopyrimidin-4-yl)-1H-pyrrolo[2,3-b]pyridin-4-yl]-2,3-dihydro-1H-indol-2-yl}methanol;
4-[5-(methyloxy)-1H-pyrrolo[2,3-b]pyridin-3-yl]pyrimidin-2-amine;
{(2S)-1-[3-(6-aminopyrimidin-4-yl)-1H-pyrrolo[2,3-b]pyridin-4-yl]pyrrolidin-2-yl}methanol;
4-{4-[2-(2-thienyl)pyrrolidin-1-yl]-1H-pyrrolo[2,3-b]pyridin-3-yl}pyrimidin-2-amine;
4-[4-(2-phenylpyrrolidin-1-yl)-1H-pyrrolo[2,3-b]pyridin-3-yl]pyrimidin-2-amine;
4-(4-{(3R)-3-[(2,2-difluoroethyl)oxy]pyrrolidin-1-yl}-1H-pyrrolo[2,3-b]pyridin-3-yl)pyrimidin-2-amine;
4-(5-fluoro-1H-pyrrolo[2,3-b]pyridin-3-yl)pyrimidin-2-amine;
3-(2-aminopyrimidin-4-yl)-N-[(1-ethylpyrrolidin-2-yl)methyl]-1H-pyrrolo[2,3-b]pyridin-4-amine;
(4aR,8aR)-6-[3-(2-aminopyrimidin-4-yl)-1H-pyrrolo[2,3-b]pyridin-4-yl]hexahydro-1H-pyrido[3,4-b][1,4]oxazin-2(3H)-one;
2-[3-(2-aminopyrimidin-4-yl)-1H-pyrrolo[2,3-b]pyridin-4-yl]hexahydrocyclopenta[c]pyrrol-5(1H)-one oxime;
4-[4-(1H-pyrazol-4-yl)-1H-pyrrolo[2,3-b]pyridin-3-yl]pyrimidin-2-amine;
4-{4-[6-(methyloxy)pyridin-3-yl]-1H-pyrrolo[2,3-b]pyridin-3-yl}pyrimidin-2-amine;
3-(2-aminopyrimidin-4-yl)-N,N-dimethyl-5-(methyloxy)-1H-pyrrolo[2,3-b]pyridin-4-amine;
4-[4-chloro-5-(methyloxy)-1H-pyrrolo[2,3-b]pyridin-3-yl]pyrimidin-2-amine;
4-[5-(methyloxy)-4-pyrrolidin-1-yl-1H-pyrrolo[2,3-b]pyridin-3-yl]pyrimidin-2-amine;
2-[3-(2-aminopyrimidin-4-yl)-1H-pyrrolo[2,3-b]pyridin-4-yl]-1,2,3,4-tetrahydroisoquinolin-8-ol;
4-[4-(2-oxa-6-azaspiro[3.3]hept-6-yl)-1H-pyrrolo[2,3-b]pyridin-3-yl]pyrimidin-2-amine;
4-{4-[2-(methyloxy)pyridin-4-yl]-1H-pyrrolo[2,3-b]pyridin-3-yl}pyrimidin-2-amine;
4-(5-{[2-(methyloxy)ethyl]oxy}-1H-pyrrolo[2,3-b]pyridin-3-yl)pyrimidin-2-amine;
1-(1H-pyrrolo[2,3-b]pyridin-3-ylmethyl)guanidine;
4-[4-(2,6-diazaspiro[3.3]hept-2-yl)-1H-pyrrolo[2,3-b]pyridin-3-yl]pyrimidin-2-amine;
4-[3-(2-aminopyrimidin-4-yl)-1H-pyrrolo[2,3-b]pyridin-4-yl]pyridin-2-ol;
4-[4-(6-methyl-2,6-diazaspiro[3.3]hept-2-yl)-1H-pyrrolo[2,3-b]pyridin-3-yl]pyrimidin-2-amine;
4-{4-[(2R,5S)-2,5-dimethylpiperazin-1-yl]-1H-pyrrolo[2,3-b]pyridin-3-yl}pyrimidin-2-amine;
4-(5-{[2-(dimethylamino)ethyl]oxy}-1H-pyrrolo[2,3-b]pyridin-3-yl)pyrimidin-2-amine;
4-(4-chloro-1H-pyrrolo[2,3-b]pyridin-3-yl)-1,3-thiazol-2-amine;
3-(2-aminopyrimidin-4-yl)-1H-pyrrolo[2,3-b]pyridin-6-ol;
(3R,4R)-3-amino-1-[3-(2-aminopyrimidin-4-yl)-1H-pyrrolo[2,3-b]pyridin-4-yl]piperidin-4-ol;
{2-[3-(2-aminopyrimidin-4-yl)-1H-pyrrolo[2,3-b]pyridin-4-yl]-2,3-dihydro-1H-isoindol-1-yl}methanol;
1,1-dimethylethyl[(3R,4R)-1-[3-(2-aminopyrimidin-4-yl)-1H-pyrrolo[2,3-b]pyridin-4-yl]-4-(methyloxy)pyrrolidin-3-yl]methylcarbamate;
2-(1H-pyrrolo[2,3-b]pyridin-3-yl)-1,3-thiazol-4-amine;
4-[1-(phenylmethyl)-2-({2-[(phenylmethyl)oxy]ethyl}oxy)-1,6-dihydroimidazo[4,5-d]pyrrolo[2,3-b]pyridin-8-yl]pyrimidin-2-amine;
3-(2-aminopyrimidin-4-yl)-N-(2-pyrrolidin-1-ylethyl)-1H-pyrrolo[2,3-b]pyridin-4-amine;
N′-[3-(2-aminopyrimidin-4-yl)-1H-pyrrolo[2,3-b]pyridin-4-yl]-N,N-diethylethane-1,2-diamine;
N′-[3-(2-aminopyrimidin-4-yl)-1H-pyrrolo[2,3-b]pyridin-4-yl]-N,N-dimethylpropane-1,3-diamine;
3-(2-aminopyrimidin-4-yl)-N-(2-piperidin-1-ylethyl)-1H-pyrrolo[2,3-b]pyridin-4-amine;
4-{4-[2-(trifluoromethyl)phenyl]-1H-pyrrolo[2,3-b]pyridin-3-yl}pyrimidin-2-amine;
2-[3-(2-aminopyrimidin-4-yl)-1H-pyrrolo[2,3-b]pyridin-4-yl]benzonitrile;
{(2R)-1-[3-(2-aminopyrimidin-4-yl)-1H-pyrrolo[2,3-b]pyridin-4-yl]-3,3-difluoropyrrolidin-2-yl}methanol;
N-[3-(2-aminopyrimidin-4-yl)-1H-pyrrolo[2,3-b]pyridin-4-yl]-H-methylethane-1,2-diamine;
N-[3-(2-aminopyrimidin-4-yl)-1H-pyrrolo[2,3-b]pyridin-4-yl]ethane-1,2-diamine;
3-(2-aminopyrimidin-4-yl)-N-(2-pyridin-2-ylethyl)-1H-pyrrolo[2,3-b]pyridin-4-amine;
4-(1H-pyrrolo[2,3-b]pyridin-4-yl)pyrimidin-2-amine;
N-[3-(2-aminopyrimidin-4-yl)-1H-pyrrolo[2,3-b]pyridin-4-yl]propane-1,3-diamine;
3-(2-aminopyrimidin-4-yl)-N-(2-fluoroethyl)-1H-pyrrolo[2,3-b]pyridin-4-amine;
(2R)-2-{[3-(2-aminopyrimidin-4-yl)-1H-pyrrolo[2,3-b]pyridin-4-yl]amino}butan-1-ol;
(2S)-2-{[3-(2-aminopyrimidin-4-yl)-1H-pyrrolo[2,3-b]pyridin-4-yl]amino}butan-1-ol;
3-(2-aminopyrimidin-4-yl)-N-(pyrrolidin-3-ylmethyl)-1H-pyrrolo[2,3-b]pyridin-4-amine;
3-(2-aminopyrimidin-4-yl)-N-(pyrrolidin-2-ylmethyl)-1H-pyrrolo[2,3-b]pyridin-4-amine;
3-(2-aminopyrimidin-4-yl)-N-(2-pyridin-4-ylethyl)-1H-pyrrolo[2,3-b]pyridin-4-amine;
N-[3-(2-aminopyrimidin-4-yl)-1H-pyrrolo[2,3-b]pyridin-4-yl]-H-methylpropane-1,3-diamine;
N-[3-(2-aminopyrimidin-4-yl)-1H-pyrrolo[2,3-b]pyridin-4-yl]cyclohexane-1,3-diamine;
N-[3-(2-aminopyrimidin-4-yl)-1H-pyrrolo[2,3-b]pyridin-4-yl]benzene-1,3-diamine;
(1R,2R)—N-[3-(2-aminopyrimidin-4-yl)-1H-pyrrolo[2,3-b]pyridin-4-yl]cyclohexane-1,2-diamine;
N-[3-(2-aminopyrimidin-4-yl)-1H-pyrrolo[2,3-b]pyridin-4-yl]benzene-1,2-diamine;
3-(2-aminopyrimidin-4-yl)-N-(2-methyl-2-pyrrolidin-1-ylpropyl)-1H-pyrrolo[2,3-b]pyridin-4-amine;
N-[3-(2-aminopyrimidin-4-yl)-1H-pyrrolo[2,3-b]pyridin-4-yl]benzene-1,4-diamine;
3-(2-aminopyrimidin-4-yl)-N-[(1-methyl-1H-imidazol-2-yl)methyl]-1H-pyrrolo[2,3-b]pyridin-4-amine;
3-(2-aminopyrimidin-4-yl)-N-[(2S)-pyrrolidin-2-ylmethyl]-1H-pyrrolo[2,3-b]pyridin-4-amine;
3-(2-aminopyrimidin-4-yl)-N-[(2R)-pyrrolidin-2-ylmethyl]-1H-pyrrolo[2,3-b]pyridin-4-amine;
phenylmethyl[(2R)-2-{[3-(2-aminopyrimidin-4-yl)-1H-pyrrolo[2,3-b]pyridin-4-yl]amino}-3-hydroxypropyl]carbamate;
N²-[3-(2-aminopyrimidin-4-yl)-1H-pyrrolo[2,3-b]pyridin-4-yl]-N¹,N¹-dimethylpropane-1,2-diamine;
(2S)-2-{[3-(2-aminopyrimidin-4-yl)-1H-pyrrolo[2,3-b]pyridin-4-yl]amino}-3-(dimethylamino)propan-1-ol;
3-(2-aminopyrimidin-4-yl)-N-(2-piperidin-2-ylethyl)-1H-pyrrolo[2,3-b]pyridin-4-amine;
1H,1′H-4,4′-bipyrrolo[2,3-b]pyridine;
N-[3-(2-aminopyrimidin-4-yl)-1H-pyrrolo[2,3-b]pyridin-4-yl]-N′-ethylethane-1,2-diamine;
4-(1H-indol-5-yl)-1H-pyrrolo[2,3-b]pyridine;
4-(1H-indol-4-yl)-1H-pyrrolo[2,3-b]pyridine;
4-(5-chloro-1H-pyrrolo[2,3-b]pyridin-4-yl)pyridin-2-amine;
4(7-phenyl-3H-imidazo[4,5-b]pyridin-2-yl)aniline;
N²,N²-dimethyl-N-[3({4-[4-(methyloxy)-1H-pyrrolo[2,3-b]pyridin-2-yl]pyrimidin-2-yl}amino)propyl]glycinamide;
2(6-chloropyridin-3-yl)-7-phenyl-3H-imidazo[4,5-b]pyridine;
4-[2-(2-aminopyridin-4-yl)-3H-imidazo[4,5-b]pyridin-7-yl]benzamide;
5(7-phenyl-3H-imidazo[4,5-b]pyridin-2-yl)pyridin-2-amine;
4-[2-(6-aminopyridin-3-yl)-3H-imidazo[4,5-b]pyridin-7-yl]benzamide;
4(7-phenyl-3H-imidazo[4,5-b]pyridin-2-yl)pyridin-2-amine;
4-(4-phenyl-1H-pyrrolo[2,3-b]pyridin-2-yl)pyrimidin-2-amine;
{3-[2-(2-aminopyrimidin-4-yl)-1H-pyrrolo[2,3-b]pyridin-4-yl]phenyl}methanol;
4(4-pyridin-4-yl-1H-pyrrolo[2,3-b]pyridin-2-yl)pyrimidin-2-amine;
4-bromo-2-(1H-pyrazol-4-yl)-1H-pyrrolo[2,3-b]pyridine;
3-[2-(2-aminopyrimidin-4-yl)-1H-pyrrolo[2,3-b]pyridin-4-yl]-N,N-dimethylbenzamide;
4-{4-[3-(chloromethyl)phenyl]-1H-pyrrolo[2,3-b]pyridin-2-yl}pyrimidin-2-amine;
4(4-phenyl-1H-pyrrolo[2,3-b]pyridin-2-yl)pyridin-2-amine;
4-[2-(1H-pyrazol-4-yl)-1H-pyrrolo[2,3-b]pyridin-4-yl]pyridin-2-amine;
4-[4-(2-aminopyridin-4-yl)-1H-pyrrolo[2,3-b]pyridin-2-yl]pyrimidin-2-amine;
3-[4-(2-aminopyridin-4-yl)-1H-pyrrolo[2,3-b]pyridin-2-yl]benzenesulfonamide;
3,3′-(1H-pyrrolo[2,3-b]pyridine-2,4-diyl)dibenzenesulfonamide;
2-[4-(2-aminopyridin-4-yl)-1H-pyrrolo[2,3-b]pyridin-2-yl]acetamide;
4-[2-(1H-indazol-6-yl)-1H-pyrrolo[2,3-b]pyridin-4-yl]pyridin-2-amine;
3-[4-(1H-indol-4-yl)-1H-pyrrolo[2,3-b]pyridin-2-yl]benzenesulfonamide;
4,4′-(1H-pyrrolo[2,3-b]pyridine-2,4-diyl)dipyridin-2-amine;
N-(3-{2-[3-(aminosulfonyl)phenyl]-1H-pyrrolo[2,3-b]pyridin-4-yl}phenyl)acetamide;
4-[4-(4-fluorophenyl)-1H-pyrrolo[2,3-b]pyridin-2-yl]pyridin-2-amine;
4-(4-chloro-1H-pyrrolo[2,3-b]pyridin-2-yl)pyridin-2-amine;
N-{3-[2-(1H-pyrazol-4-yl)-1H-pyrrolo[2,3-b]pyridin-4-yl]phenyl}acetamide;
N-{3-[2-(1H-indazol-6-yl)-1H-pyrrolo[2,3-b]pyridin-4-yl]phenyl}acetamide;
{3-[4-(2-aminopyridin-4-yl)-1H-pyrrolo[2,3-b]pyridin-2-yl]phenyl}methanol;
3-[4-(1H-indol-4-yl)-1H-pyrrolo[2,3-b]pyridin-2-yl]-N-(phenylmethyl)benzenesulfonamide;
4-[4-(1H-indol-4-yl)-1H-pyrrolo[2,3-b]pyridin-2-yl]pyridin-2-amine;
N-(3-hydroxypropyl)-3-[4-(1H-indol-4-yl)-1H-pyrrolo[2,3-b]pyridin-2-yl]benzenesulfonamide;
2-{4-[3-(2-aminopyrimidin-4-yl)-1H-pyrrolo[2,3-b]pyridin-4-yl]-2-oxopiperazin-1-yl}-N-(2-methylpropyl)acetamide;
(4S)-1-[3-(2-aminopyrimidin-4-yl)-1H-pyrrolo[2,3-b]pyridin-4-yl]-4-fluoro-L-proline;
4-{4-[(2S)-2-(pyrrolidin-1-ylmethyl)pyrrolidin-1-yl]-1H-pyrrolo[2,3-b]pyridin-3-yl}pyrimidin-2-amine;
(3S)-1-[3-(2-aminopyrimidin-4-yl)-1H-pyrrolo[2,3-b]pyridin-4-yl]pyrrolidine-3-carboxylic acid;
5-[3-(2-aminopyrimidin-4-yl)-1H-pyrrolo[2,3-b]pyridin-4-yl]pyridin-2-ol;
4-{4-[4-(methyloxy)-1H-pyrrolo[3,2-c]pyridin-2-yl]-1H-pyrrolo[2,3-b]pyridin-3-yl}pyrimidin-2-amine; and
4-(4-{(3R)-3-[(1-methylethyl)oxy]pyrrolidin-1-yl}-1H-pyrrolo[2,3-b]pyridin-3-yl)pyrimidin-2-amine.

15. A compound of Formula IC: 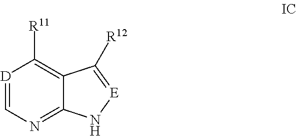
or a pharmaceutically acceptable salt thereof, wherein
R¹¹ is alkoxy, hydroxyalkoxy or alkoxyalkoxy;
R¹² is a (5-9 membered)heteroaryl or phenyl, wherein the (5-9 membered)heteroaryl or phenyl are each optionally substituted with 1 or 2 substituents independently selected from amine, alkoxy, hydroxyl, amine, alkyl, aminocarbonyl and halo; and
D and E are independently selected from N and CH, provided that the compound of formula IC is not one of the following compounds:

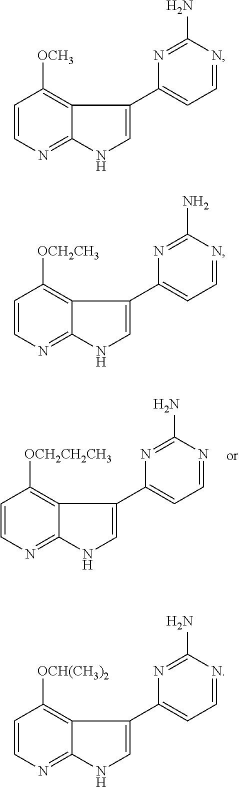

16. A compound according to claim 15 selected from one of the following compounds, or a pharmaceutically acceptable salt thereof:
4-chloro-6-[4-(methyloxy)-1H-pyrrolo[2,3-b]pyridin-3-yl]pyrimidin-2-amine;
4-(methyloxy)-3-[2-(4-methylpiperazin-1-yl)pyridin-4-yl]-1H-pyrrolo[2,3-b]pyridine;
2-fluoro-5-[4-(methyloxy)-1H-pyrrolo[2,3-b]pyridin-3-yl]aniline; 4′-chloro-4-(methyloxy)-1H,1′H-3,5′-bipyrrolo[2,3-b]pyridine;
4-(methyloxy)-3-pyridin-4-yl-1H-pyrazolo[3,4-d]pyrimidine;
4-[4-(methyloxy)-1H-pyrazolo[3,4-d]pyrimidin-3-yl]pyridin-2-amine;
4-(methyloxy)-3-pyridin-3-yl-1H-pyrazolo[3,4-d]pyrimidine;
4-[4-(methyloxy)-7H-pyrrolo[2,3-d]pyrimidin-5-yl]pyridin-2-amine; and
4-[4-(ethyloxy)-1H-pyrrolo[2,3-b]pyridin-3-yl]-6-methylpyrimidin-2-amine.

17. A pharmaceutical composition comprising a compound according to claim 1, or a pharmaceutically acceptable salt thereof, and a pharmaceutically acceptable carrier, excipient or diluent.

18. A method of modulating CDK in a cell, comprising contacting a cell in which inhibition of CDK is desired with a compound according to claim 1, or a pharmaceutically acceptable salt thereof.

19. A method of treating a disease or condition that involves CDK, comprising administering to a mammal, in need of the treatment, a compound according to claim 1, or a pharmaceutically acceptable salt thereof.

20. The method according to claim 19, wherein the disease or condition is selected from chronic lymphocytic lymphoma, Burkitts lymphoma mantle cell lymphoma, multiple myeloma, breast cancer, small cell lung carcinoma, eosophageal carcinoma, HIV, HTLV associated leukemia, rheumatoid arthritis, multiple sclerosis, transplant rejection and cardiac hypertrophy.
